(19) United States
(12) Patent Application Publication
(10) Pub. No.: US 2024/0190849 A1
(43) Pub. Date: Jun. 13, 2024

(54) AMINOPYRIMIDINE COMPOUNDS AND METHODS OF THEIR USE

(71) Applicant: TYRA BIOSCIENCES, INC., Carlsbad, CA (US)

(72) Inventors: Robert L. HUDKINS, Carlsbad, CA (US); Daniel C. BENSEN, Carlsbad, CA (US)

(21) Appl. No.: 18/547,893

(22) PCT Filed: Feb. 25, 2022
(86) PCT No.: PCT/US2022/017873

§ 371 Date: Aug. 25, 2023

Related U.S. Application Data
(60) Provisional application No. 63/154,409, filed on Feb. 26, 2021.
(60) Provisional application No. 63/216,868, filed on Jun. 30, 2021.
(60) Provisional application No. 63/303,284, filed on Jan. 26, 2022.

(51) Int. Cl. C07D 403/14; A61P 35/00; C07D 401/14; C07D 403/04; C07D 405/14; C07D 413/14; C07D 417/14; C07D 471/04; C07D 487/04

(52) U.S. Cl. CPC C07D 403/14; A61P 35/00; C07D 401/14; C07D 403/04; C07D 405/14; C07D 413/14; C07D 417/14; C07D 471/04; C07D 487/04

(57) ABSTRACT

Disclosed herein are substituted aminopyrimidine compounds of formula I: and pharmaceutically acceptable salts thereof, wherein Ar, R¹, and R² have the meanings described herein; pharmaceutical compositions that include such compounds, and methods of using such compounds.

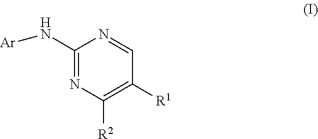

### CROSS-REFERENCE TO RELATED APPLICATIONS

This application claims the benefit of U.S. Provisional Patent Application No. 63/154,409, filed Feb. 26, 2021, U.S. Provisional Patent Application No. 63/216,868, filed Jun. 30, 2021, and U.S. Provisional Patent Application No. 63/303,284, filed Jan. 26, 2022. Each of the aforementioned applications is incorporated by reference herein in its entirety.

### TECHNICAL FIELD

Disclosed herein are substituted aminopyrimidine compounds, pharmaceutical compositions that include such compounds, and methods of using such compounds.

### BACKGROUND

Various approaches have been employed in the past to block the activity of tyrosine kinases. These kinase inhibitors are often small molecules. These small molecules can be used to target these kinases to block the development, growth or spread of cancer.

Fibroblast growth factors (FGFs) and their receptors (FGFRs) regulate a wide range of physiologic cellular processes, such as embryonic development, differentiation, proliferation, survival, migration, and angiogenesis. The FGF family comprises 18 secreted ligands (FGFs) which are readily sequestered to the extracellular matrix by heparin sulfate proteoglycans (HPSGs). For signal propagation, FGFs are released from the extracellular matrix by proteases or specific FGF-binding proteins, with the liberated FGFs subsequently binding to a cell surface FGF-receptor (FGFR) in a ternary complex consisting of FGF, FGFR and HPSG (Beenken, A., Nat. Rev. Drug Discov. 2009; 8:235-253).

FGFR signaling components are frequently altered in human cancer, and several preclinical models have provided compelling evidence for the oncogenic potential of aberrant FGFR signaling in carcinogenesis, thereby validating FGFR signaling as an attractive target for cancer treatment.

Compounds that inhibit FGFR are needed.

### SUMMARY

The present disclosure provides compounds that inhibit FGFR. In particular, the disclosure provides compounds of formula I:

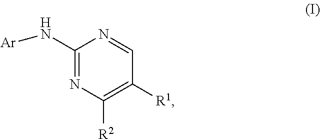

or pharmaceutically acceptable salts thereof,
wherein:
   Ar is a 6-10-membered aryl group or a 5-10-membered heteroaryl group, each optionally substituted;
   R¹ is H, F, Cl, Br, C₁₋₆alkyl, CN, C₁₋₆haloalkyl, —C(O)NH₂, —C(O)NH(C₁-6alkyl), or —C(O)N(C₁₋₆alkyl)₂;
   R² is a:
      9-membered bicyclic heteroaryl comprising a 6-5-fused ring system wherein the 5-membered ring contains at least one nitrogen atom and the 6 membered ring is substituted with at least one E; or
      13-membered tricyclic heteroaryl comprising a 6-5-6-fused ring system wherein the 5-membered ring contains at least one nitrogen atom and one of the 6 membered rings is substituted with at least one E; or
      12- or 13-membered tricyclic group comprising a 5- or 6-membered cycloalkyl or heterocycloalkyl ring fused to a 9-membered bicyclic heteroaryl comprising a 6-5-fused ring system wherein the 5-membered ring contains at least one nitrogen atom and the 6 membered ring is substituted with at least one E;
   wherein E is an electrophilic moiety that is capable of reacting with a nucleophile so as to form a covalent bond between an atom of the nucleophile and an atom of the electrophilic moiety.

Pharmaceutical compositions and methods of using and making the compounds of the disclosure are also provided.

### DETAILED DESCRIPTION OF ILLUSTRATIVE EMBODIMENTS

The following terms are used to describe the present disclosure. In instances where a term is not specifically defined herein, that term is given an art-recognized meaning by those of ordinary skill applying that term in context to its use in describing the present disclosure.

The term “optionally substituted,” as used herein to describe a substituent defined herein, means that the substituent may be, but is not required to be, substituted with one or more of: halo (i.e., —F, —Cl, —Br, —I), cyano (—CN), —OH, —C₁-C₆alkyl, C₃-C₆cycloalkyl, 5-7 membered heterocycloalkyl, 5-7 membered spirocycloalkyl, or 5-7 membered spiroheterocycloalkyl, bridged cycloalkyl, bridged heterocycloalkyl, C₂-C₆alkenyl, C₂-C₆ alkynyl, C₁-C₆haloalkyl (e.g., —CF₃; —CHF₂, —CH₂CF₃, and the like), —C₁-C₆alkoxy (e.g., —OC₁-C₆alkyl; —O(optionally substituted C₁-C₆alkyl)), —C₁-C₆ haloalkoxy (e.g., —OCF₃; —OCHF₂, —OCH₂CF₃, and the like), C₁-C₆alkylthio (e.g., —SCH₃; —SCH₂CH₃, and the like), C₁-C₆alkylamino (e.g., —CH₂NH₂, —CH₂CH₂NH₂, and the like), —NH₂, —NH(C₁-C₆alkyl), —N(C₁-C₆alkyl)₂ (e.g., —N(C₁-C₆alkyl)(optionally substituted C₁-C₆alkyl)), —NH(C₁-C₆alkoxy), —C(O)NHC₁-C₆alkyl, —C(O)N(C₁-C₆alkyl)₂, —COOH, —C₁-C₆alkylCOOH, —C₃-C₆cycloalkylCOOH, —C(O)NH₂, —C₁-C₆alkylCONH₂, —C₃-C₆cycloalkylCONH₂, C₁-C₆alkylCONHC₁-C₆alkyl, C₁-C₆alkylCON(C₁-C₆alkyl)₂, —C(O)C₁-C₆ alkyl, —C(O)OC₁-C₆ alkyl, —NHCO(C₁-C₆ alkyl), —N(C₁-C₆ alkyl)C(O)(C₁-C₆ alkyl), —S(O)C₁-C₆ alkyl, —S(O)₂C₁-C₆ alkyl, oxo (i.e., ═O), —NHSO₂(C₁-C₆ alkyl), —N(C₁-C₆ alkyl)SO₂(C₁-C₆ alkyl), SO₂NH(C₁-C₆ alkyl), SO₂N(C₁-C₆ alkyl)₂, 6-12 membered aryl, 4-6-membered heterocycloalkyl, or 5 to 12 membered heteroaryl groups. In some embodiments, each of the above optional substituents are themselves optionally substituted by one or two of these groups.

In some embodiments wherein an optionally substituted alkyl group (e.g., optionally substituted C₁-C₆alkyl) is substituted with halogen, the group is referred to as a haloalkyl group.

In some embodiments, excluded from substituents encompassed by “optionally substituted” are substituents that have the following general formula:

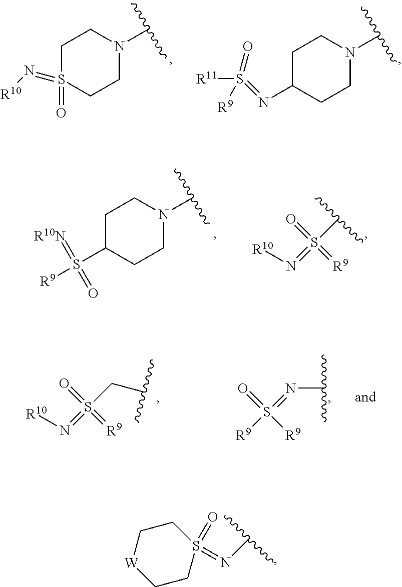

wherein W is —CH₂—, —C(O)—, —CH(OH)— or —N(R¹⁰)—; R¹⁰ is —H, optionally substituted C₁-C₆-alkyl, optionally substituted C₁-C₆-alkenyl, or —C₃-C₆-cycloalkyl; and each R⁹ is independently optionally substituted C₁-C₆-alkyl, optionally substituted C₁-C₆-alkenyl, or C₃-C₆-cycloalkyl.

When a range of carbon atoms is used herein, for example, “C₁-C₆” (or equivalently “C₁₋₆”), all ranges, as well as individual numbers of carbon atoms are encompassed. For example, “C₁-C₃” includes C₁-C₃, C₁-C₂, C₂-C₃, C₁, C₂, and C₃. Thus, for example, a “C₁ to C₄ alkyl” group refers to all alkyl groups having from 1 to 4 carbons (e.g., 1, 2, 3, or 4), that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. A “C₁ to C₆ alkyl” group refers to all alkyl groups having from 1 to 6 carbons (e.g., 1, 2, 3, 4, 5, or 6).

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms, although the present definition also covers the occurrence of the term “alkyl” where no numerical range is designated). The “alkyl” group may also be a medium size alkyl having 1 to 12 carbon atoms. The “alkyl” group could also be a lower alkyl having 1 to 6 carbon atoms. An alkyl group may be substituted or unsubstituted. By way of example only, “C₁-C₅ alkyl” indicates that there are one to five carbon atoms in the alkyl chain, i.e., the alkyl chain is selected from methyl, ethyl, n-propyl, isopropyl, n-butyl, isobutyl, tert-butyl, pentyl (branched and straight-chained), etc. Typical alkyl groups include, but are in no way limited to, methyl, ethyl, propyl, isopropyl, butyl, isobutyl, tertiary butyl, pentyl and hexyl. In several embodiments, “Me” is methyl (e.g., CH₃).

The term “C₁-C₆alk” when used alone or as part of a substituent group refers to an aliphatic linker having 1, 2, 3, 4, 5, or 6 carbon atoms and includes, for example, —CH₂—, —CH(CH₃)—, —CH(CH₃)—CH₂—, and —C(CH₃)₂—. The term “-C₀alk-” refers to a bond.

As used herein, “alkenyl” refers to an alkyl group that contains in the straight or branched hydrocarbon chain one or more double bonds. An alkenyl group may be unsubstituted or substituted.

As used herein, “alkynyl” refers to an alkyl group that contains in the straight or branched hydrocarbon chain one or more triple bonds. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups may contain between 3 and 12 carbon atoms. For example, a C₃-C₆cycloalkyl group indicates that there three to six carbon atoms in the ring, that is, the ring is a cyclopropyl, cyclobutyl, cyclopentyl, or cyclohexyl group. A cycloalkyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

The term “haloalkyl” refers to an alkyl group wherein one or more of the hydrogen atoms has been replaced with one or more halogen atoms. Halogen atoms include chlorine, fluorine, bromine, and iodine. Examples of haloalkyl groups of the disclosure include, for example, trifluoromethyl (—CF₃), chloromethyl (—CH₂Cl), and the like.

As used herein, “heteroaryl” refers to a monocyclic or multicyclic aromatic ring system (a ring system with fully delocalized pi-electron system) that contain(s) one or more heteroatoms, that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine. Heteroaryl rings may also include bridge head nitrogen atoms. For example but not limited to: pyrazolo[1,5-a]pyridine, imidazo[1,2-a]pyridine, and pyrazolo[1,5-a]pyrimidine. A heteroaryl group may be substituted or unsubstituted.

As used herein, “heterocycloalkyl” refers to three-, four-, five-, six-, seven-, eight-, nine-, ten-, up to 18-membered monocyclic, bicyclic, and tricyclic ring system wherein carbon atoms together with from 1 to 5 heteroatoms constitute said ring system. A heterocycloalkyl may optionally contain one or more unsaturated bonds situated in such a way, however, that a fully delocalized pi-electron system does not occur throughout all the rings. The heteroatom(s) is an element other than carbon including, but not limited to, oxygen, sulfur, and nitrogen. A heterocycloalkyl may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogen atom in a heterocycloalkyl may be quaternized. Heterocycloalkyl groups may be unsubstituted or substituted. Examples of such “heterocycloalkyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline, 3,4-methylenedioxyphenyl).

As used herein, the term “amino” refers to a —NH₂ group.

As used herein, the term “hydroxy” refers to a —OH group.

As used herein, the term “halo” or “halogen” refers to an atom that is fluorine, chlorine, bromine and/or iodine.

As used herein, “alkoxy” and “alkylthio” (or thioalkoxy) refer to alkyl groups attached to the remainder of a molecule via an oxygen atom or a sulfur atom, respectively.

The term “pharmaceutically acceptable salt” refers to a salt of a compound that does not cause significant irritation to an organism to which it is administered and does not abrogate the biological activity and properties of the compound. In several embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Thus, pharmaceutically acceptable salts include, but are not limited to, acetate, aspartate, benzenesulfonate, benzoate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, acetate, succinate, sulfate, tartrate, teoclate, and tosylate salts. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)-methylamine, C₁-C₇ alkylamine, cyclohexylamine, triethanolamine, ethylenediamine, and salts with amino acids such as arginine and lysine.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof. It is also understood that, in any compound described herein, all isotopes of the included atoms are envisioned. For example, any instance of hydrogen, may include hydrogen-1 (protium), hydrogen-2 (deuterium), hydrogen-3 (tritium) or other isotopes; any instance of carbon may include carbon-12, carbon-13, carbon-14, or other isotopes; any instance of oxygen may include oxygen-16, oxygen-17, oxygen-18, or other isotopes; any instance of fluorine may include one or more of fluorine-18, fluorine-19, or other isotopes; any instance of sulfur may include one or more of sulfur-32, sulfur-34, sulfur-35, sulfur-36, or other isotopes.

The term “gatekeeper mutation” when used herein denotes mutations in a kinase enzyme that modulate the accessibility of the kinase ATP-binding pocket.

The term “target sequence” or “target nucleic acid sequence” shall be given its ordinary meaning and shall also include and also refer to the particular nucleotide sequence of the target nucleic acid to be detected (e.g., through amplification). The target sequence may include a probe-hybridizing region contained within the target molecule with which a probe will form a stable hybrid under desired conditions. The “target sequence” may also include the complexing sequences to which the oligonucleotide primers complex and be extended using the target sequence as a template. Where the target nucleic acid is originally single-stranded, the term “target sequence” also refers to the sequence complementary to the “target sequence” as present in the target nucleic acid. If the “target nucleic acid” is originally double-stranded, the term “target sequence” refers to both the plus (+) and minus (−) strands.

As used herein, the term “kinase inhibitor” means any compound, molecule or composition that inhibits or reduces the activity of a kinase. The inhibition can be achieved by, for example, blocking phosphorylation of the kinase (e.g., competing with adenosine triphosphate (ATP), a phosphorylating entity), by binding to a site outside the active site, affecting its activity by a conformational change, or by depriving kinases of access to the molecular chaperoning systems on which they depend for their cellular stability, leading to their ubiquitylation and degradation.

As used herein, “subject,” “host,” “patient,” and “individual” are used interchangeably and shall be given its ordinary meaning and shall also refer to an organism that has FGFR proteins. This includes mammals, e.g., a human, a non-human primate, ungulates, canines, felines, equines, mice, rats, and the like. The term “mammal” includes both human and non-human mammals. In some aspects, the “subject,” “host,” “patient,” or “individual” is human.

“Diagnosis” as used herein shall be given its ordinary meaning and shall also include determination of a subject's susceptibility to a disease or disorder, determination as to whether a subject is presently affected by a disease or disorder, prognosis of a subject affected by a disease or disorder (e.g., identification of cancer or cancerous states, stages of cancer, or responsiveness of cancer to therapy), and use of therametrics (e.g., monitoring a subject's condition to provide information as to the effect or efficacy of therapy).

The term “sample” or “biological sample” shall be given its ordinary meaning and also encompasses a variety of sample types obtained from an organism and can be used in an imaging, a diagnostic, a prognostic, or a monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “treatment,” “treating,” “treat” and the like shall be given its ordinary meaning and shall also include herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein shall be given its ordinary meaning and shall also cover any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, e.g., arresting its development; and/or (c) relieving the disease symptom, e.g., causing regression of the disease or symptom.

The terms “cancer,” “neoplasm,” and “tumor” are used interchangeably herein, shall be given its ordinary meaning and shall also refer to cells which exhibit relatively autonomous growth, so that they exhibit an aberrant growth phenotype characterized by a significant loss of control of cell proliferation. In general, cells of interest for detection or treatment in the present application include precursors, precancerous (e.g., benign), malignant, pre-metastatic, metastatic, and non-metastatic cells. As used herein, “FGFR related cancer” denotes those cancers that involve an increased activity in a mutant FGFR kinase, for example, the continued activation of FGFR.

The term “control” refers shall be given its ordinary meaning and shall also include a sample or standard used for comparison with a sample which is being examined, processed, characterized, analyzed, etc. In several embodiments, the control is a sample obtained from a healthy patient or a non-tumor tissue sample obtained from a patient diagnosed with a tumor. In several embodiments, the control is a historical control or standard reference value or range of values. In several embodiments, the control is a comparison to a wild-type FGFR arrangement or scenario.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the general description and the following detailed description are exemplary and explanatory only and are not restrictive. The term “and/or” denotes that the provided possibilities can be used together or be used in the alternative. Thus, the term “and/or” denotes that both options exist for that set of possibilities.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

In some aspects, the disclosure is directed to compounds of formula I:

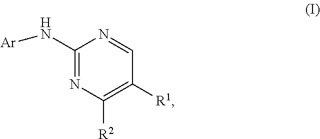

or pharmaceutically acceptable salts thereof,
wherein:
Ar is a 6-10-membered aryl group or a 5-10-membered heteroaryl group, each optionally substituted;
R¹ is H, F, Cl, Br, C₁₋₆alkyl, CN, C₁₋₆haloalkyl, —C(O)NH₂, —C(O)NH(C₁₋₆alkyl), or —C(O)N(C₁₋₆alkyl)₂;
R² is a 9-membered bicyclic heteroaryl comprising a 6-5-fused ring system wherein the 5-membered ring contains at least one nitrogen atom and the 6 membered ring is substituted with at least one E; or a 13-membered tricyclic heteroaryl comprising a 6-5-6-fused ring system wherein the 5-membered ring contains at least one nitrogen atom and one of the 6 membered rings is substituted with at least one E; or a 12- or 13-membered tricyclic group comprising a 5- or 6-membered cycloalkyl or heterocycloalkyl ring fused to a 9-membered bicyclic heteroaryl comprising a 6-5-fused ring system; wherein the 5-membered ring contains at least one nitrogen atom and the 6 membered ring is substituted with at least one E; wherein E is an electrophilic moiety that is capable of reacting with a nucleophile so as to form a covalent bond between an atom of the nucleophile and an atom of the electrophilic moiety.

In some embodiments of the compounds of formula I, or pharmaceutically acceptable salts thereof, Ar is a 6-10-membered aryl group or a 5-10-membered heteroaryl group, each optionally substituted; R¹ is H, F, Cl, Br, C₁₋₆alkyl, CN, C₁₋₆haloalkyl, —C(O)NH₂, —C(O)NH(C₁₋₆alkyl), or —C(O)N(C₁₋₆alkyl)₂; R² is a 9-membered bicyclic heteroaryl comprising a 6-5-fused ring system wherein the 5-membered ring contains at least one nitrogen atom and the 6 membered ring is substituted with at least one E; wherein E is an electrophilic moiety that is capable of reacting with a nucleophile so as to form a covalent bond between an atom of the nucleophile and an atom of the electrophilic moiety.

In other embodiments of the compounds of formula I, or pharmaceutically acceptable salts thereof, Ar is a 6-10-membered aryl group or a 5-10-membered heteroaryl group, each optionally substituted; R¹ is H, F, Cl, Br, C₁₋₆alkyl, CN, C₁₋₆haloalkyl, —C(O)NH₂, —C(O)NH(C₁₋₆alkyl), or —C(O)N(C₁₋₆alkyl)₂; R² is a 13-membered tricyclic heteroaryl comprising a 6-5-6-fused ring system wherein the 5-membered ring contains at least one nitrogen atom and one of the 6 membered rings is substituted with at least one E; wherein E is an electrophilic moiety that is capable of reacting with a nucleophile so as to form a covalent bond between an atom of the nucleophile and an atom of the electrophilic moiety.

In other embodiments of the compounds of formula I, or pharmaceutically acceptable salts thereof, Ar is a 6-10-membered aryl group or a 5-10-membered heteroaryl group, each optionally substituted; R¹ is H, F, Cl, Br, C₁₋₆alkyl, CN, C₁₋₆haloalkyl, —C(O)NH₂, —C(O)NH(C₁₋₆alkyl), or —C(O)N(C₁₋₆alkyl)₂; R² is a 12- or 13-membered tricyclic group comprising a 5- or 6-membered cycloalkyl or heterocycloalkyl ring fused to a 9-membered bicyclic heteroaryl comprising a 6-5-fused ring system; wherein the 5-membered ring contains at least one nitrogen atom and the 6 membered ring is substituted with at least one E; wherein E is an electrophilic moiety that is capable of reacting with a nucleophile so as to form a covalent bond between an atom of the nucleophile and an atom of the electrophilic moiety.

In some aspects, Ar in formula I is a 6-10-membered aryl group or a 5-10-membered heteroaryl group, each optionally substituted.

In some embodiments, Ar is an optionally substituted 6-10-membered aryl group. In some embodiments, Ar is an unsubstituted 6-10-membered aryl group, for example, an unsubstituted phenyl group, or an unsubstituted naphthyl group.

In other embodiments, Ar is a substituted 6-10-membered aryl group, for example, a substituted phenyl group, or a substituted naphthyl group.

In some embodiments, Ar is an optionally substituted phenyl group.

In some embodiments, Ar is a substituted phenyl group.

In some embodiments wherein Ar is a substituted phenyl group, the phenyl group is substituted with one or more optionally substituted C₁-C₆alkyl, optionally substituted C₃-C₆cycloalkyl, optionally substituted 4-6-membered heterocycloalkyl, —N(C₁-C₆alkyl)(optionally substituted C₁-C₆alkyl), —O(optionally substituted C₁-C₆alkyl), —CN, or halogen.

In some embodiments wherein Ar is a substituted phenyl group that is substituted with one or more optionally substituted C₁-C₆alkyl, the optionally substituted C₁-C₆alkyl is —CH₃, —CH₂CH₃, isopropyl, —CH₂CF₃, —CHF₂, —CF₃, —C₁₋₆alk-cyclopropyl, —CH₂-cyclopropyl, —C₁₋₆alk-(optionally substituted 5 or 6-membered heterocycloalkyl), —CH₂-(optionally substituted 5 or 6-membered heterocycloalkyl), or —CH₂CH₂OH.

In some embodiments wherein Ar is a substituted phenyl group that is substituted with one or more optionally substituted C₁-C₆alkyl, at least one optionally substituted C₁-C₆alkyl is —CH₃.

In some embodiments wherein Ar is a substituted phenyl group that is substituted with one or more optionally substituted C₁-C₆alkyl, at least one optionally substituted C₁-C₆alkyl is —CH₂CH₃.

In some embodiments wherein Ar is a substituted phenyl group that is substituted with one or more optionally substituted C₁-C₆alkyl, at least one optionally substituted C₁-C₆alkyl is isopropyl.

In some embodiments wherein Ar is a substituted phenyl group that is substituted with one or more optionally substituted C₁-C₆alkyl, at least one substituted C₁-C₆alkyl is —CH₂CF₃.

In some embodiments wherein Ar is a substituted phenyl group that is substituted with one or more optionally substituted C₁-C₆alkyl, at least one optionally substituted C₁-C₆alkyl is —CHF₂.

In some embodiments wherein Ar is a substituted phenyl group that is substituted with one or more optionally substituted C₁-C₆alkyl, at least one optionally substituted C₁-C₆alkyl is —CF₃.

In some embodiments wherein Ar is a substituted phenyl group that is substituted with one or more optionally substituted C₁-C₆alkyl, at least one optionally substituted C₁-C₆alkyl is —CH₂-cyclopropyl.

In some embodiments wherein Ar is a substituted phenyl group that is substituted with one or more optionally substituted C₁-C₆alkyl, the optionally substituted C₁-C₆alkyl is —CH₂-(optionally substituted 5 or 6-membered heterocycloalkyl), such as, for example,

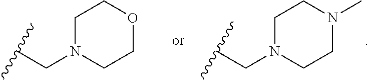

In some embodiments wherein Ar is a substituted phenyl group that is substituted with one or more optionally substituted C₁-C₆alkyl, the optionally substituted C₁-C₆alkyl is —CH₂CH₂OH.

In some embodiments wherein Ar is a substituted phenyl group that is substituted with one or more optionally substituted C₃-C₆cycloalkyl, the optionally substituted C₃-C₆cycloalkyl is cyclopropyl or cyclobutyl.

In some embodiments wherein Ar is a substituted phenyl group that is substituted with one or more optionally substituted C₃-C₆cycloalkyl, the optionally substituted C₃-C₆cycloalkyl is cyclopropyl.

In some embodiments wherein Ar is a substituted phenyl group that is substituted with one or more optionally substituted C₃-C₆cycloalkyl, the optionally substituted C₃-C₆cycloalkyl is cyclobutyl.

In some embodiments wherein Ar is a substituted phenyl group that is substituted with one or more optionally substituted 4-6-membered heterocycloalkyl, the optionally substituted 4-6-membered heterocycloalkyl is

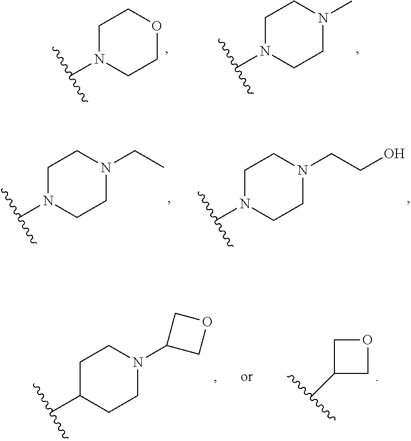

In some embodiments wherein Ar is a substituted phenyl group that is substituted with one or more optionally substituted 4-6-membered heterocycloalkyl, the optionally substituted 4-6-membered heterocycloalkyl is

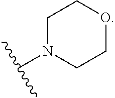 [SMILES: *N1CCOCC1]

In some embodiments wherein Ar is a substituted phenyl group that is substituted with one or more optionally substituted 4-6-membered heterocycloalkyl, the optionally substituted 4-6-membered heterocycloalkyl is

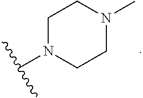 [SMILES: *N1CCN(C)CC1]

In some embodiments wherein Ar is a substituted phenyl group that is substituted with one or more optionally substituted 4-6-membered heterocycloalkyl, the optionally substituted 4-6-membered heterocycloalkyl is

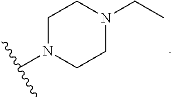 [SMILES: *N1CCN(CC)CC1]

In some embodiments wherein Ar is a substituted phenyl group that is substituted with one or more optionally substituted 4-6-membered heterocycloalkyl, the optionally substituted 4-6-membered heterocycloalkyl is

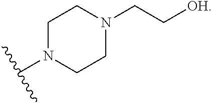 [SMILES: *N1CCN(CCO)CC1]

In some embodiments wherein Ar is a substituted phenyl group that is substituted with one or more optionally substituted 4-6-membered heterocycloalkyl, the optionally substituted 4-6-membered heterocycloalkyl is

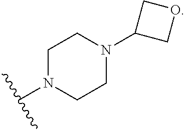 [SMILES: *N1CCN(C2COC2)CC1]

In some embodiments wherein Ar is a substituted phenyl group that is substituted with one or more optionally substituted 4-6-membered heterocycloalkyl, the optionally substituted 4-6-membered heterocycloalkyl is

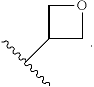 [SMILES: *C1COC1]

In some embodiments wherein Ar is a substituted phenyl group that is substituted with one or more N(C₁-C₆alkyl)(optionally substituted C₁-C₆alkyl), the N(C₁-C₆alkyl)(optionally substituted C₁-C₆alkyl) is

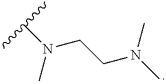 [SMILES: *N(C)CCN(C)C]

In some embodiments wherein Ar is a substituted phenyl group that is substituted with one or more —O(optionally substituted C₁-C₆alkyl), the —O(optionally substituted C₁-C₆alkyl) is —OCH₃, —OCH₂CH₃, or

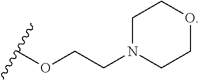 [SMILES: *OCCN1CCOCC1]

In some embodiments wherein Ar is a substituted phenyl group that is substituted with one or more —O(optionally substituted C₁-C₆alkyl), the —O(optionally substituted C₁-C₆alkyl) is —OCH₃.

In some embodiments wherein Ar is a substituted phenyl group that is substituted with one or more —O(optionally substituted C₁-C₆alkyl), the —O(optionally substituted C₁-C₆alkyl) is —OCH₂CH₃.

In some embodiments wherein Ar is a substituted phenyl group that is substituted with one or more —O(optionally substituted C₁-C₆alkyl), the —O(optionally substituted C₁-C₆alkyl) is

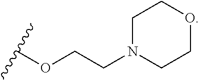 [SMILES: *OCCN1CCOCC1]

In some embodiments wherein Ar is a substituted phenyl group, the phenyl group is substituted with —CN.

In some embodiments wherein Ar is a substituted phenyl group that is substituted with one or more halogen, at least one halogen is —F.

In some embodiments, Ar in formula I is

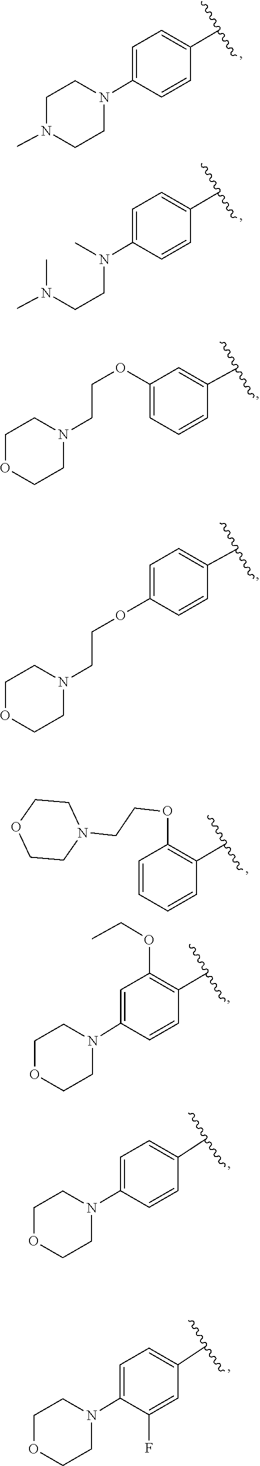

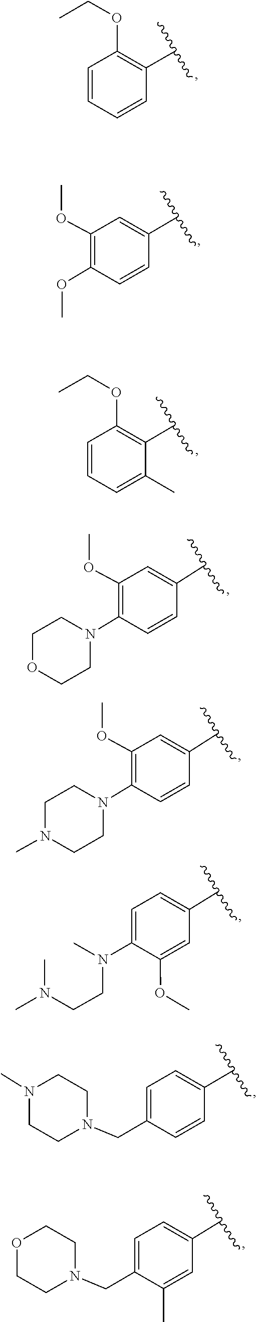

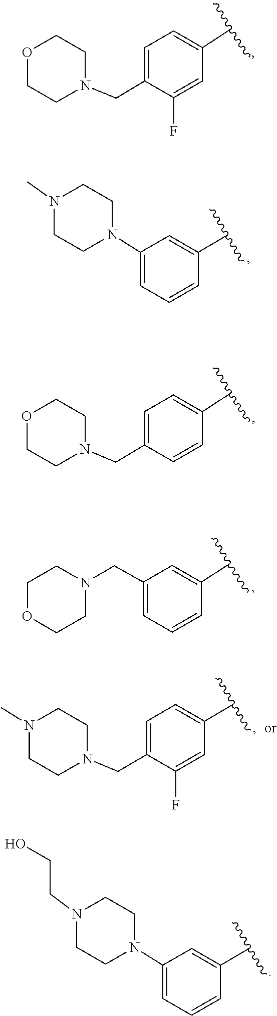 [SMILES: *c1ccc(CN2CCOCC2)c(F)c1 ; *c1cccc(N2CCN(C)CC2)c1 ; *c1ccc(CN2CCOCC2)cc1 ; *c1cccc(CN2CCOCC2)c1 ; *c1ccc(CN2CCN(C)CC2)c(F)c1 ; *c1cccc(N2CCN(CCO)CC2)c1]

In some aspects, Ar in formula I is an optionally substituted 5-10-membered heteroaryl group, such as, for example, furan, furazan, thiophene, benzothiphene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, or triazine, each optionally substituted.

In some embodiments, the optionally substituted 5-10-membered heteroaryl group is a pyridinyl, a pyrazolyl, a triazolyl, an imidazolyl, a pyrazolopyrimidine, or a triazolopyridine, each optionally substituted.

In some embodiments, Ar is an unsubstituted 5-10-membered heteroaryl group.

In some embodiments, Ar is a substituted 5-10-membered heteroaryl group.

In some embodiments, Ar is a substituted pyrazolyl group.

In some embodiments wherein Ar is a substituted 5-10-membered heteroaryl group, the substituted 5-10-membered heteroaryl group is substituted with one or more optionally substituted C₁-C₆alkyl, optionally substituted C₃-C₆cycloalkyl, optionally substituted 4-6-membered heterocycloalkyl, —N(C₁-C₆alkyl)(optionally substituted C₁-C₆alkyl), —O(optionally substituted C₁-C₆alkyl), —CN, or halogen.

In some embodiments wherein Ar is a substituted 5-10-membered heteroaryl group, the substituted 5-10-membered heteroaryl group is substituted with one or more —C(O)—O(optionally substituted C₁-C₆alkyl), such as, for example, —C(O)O-t-butyl.

In some embodiments wherein Ar is a substituted 5-10 membered heteroaryl group that is substituted with one or more optionally substituted C₁-C₆alkyl, the optionally substituted C₁-C₆alkyl is —CH₃, —CH₂CH₃, isopropyl, —CH₂CF₃, —CHF₂, —CF₃, —CH₂-cyclopropyl, —CH₂-(optionally substituted 5 or 6-membered heterocycloalkyl), or —CH₂CH₂OH.

In some embodiments wherein Ar is a substituted 5-10 membered heteroaryl group that is substituted with one or more optionally substituted C₁-C₆alkyl, the optionally substituted C₁-C₆alkyl is —CH₂CH(OH)CH₃, —C(CN)(CH₃)₂, —CH₂CH₂CH₂OH, —CH₂CH₂OCH₃, —CH₂C(OH)(CH₃)₂, —CH₂CH₂OCH₂CH₃, —CH₂CH₂OCH(CH₃)₂, CH₂CH(OCH₃)CH₃, —CH₂CHF₂, —CH₂CH₂N(CH₃)₂, —CH₂CH₂-(optionally substituted 5 or 6-membered heterocycloalkyl), or —CH₂-optionally substituted cyclopropyl.

In some embodiments wherein Ar is a substituted 5-10 membered heteroaryl group that is substituted with one or more optionally substituted C₁-C₆alkyl, at least one optionally substituted C₁-C₆alkyl is —CH₃.

In some embodiments wherein Ar is a substituted 5-10 membered heteroaryl group that is substituted with one or more optionally substituted C₁-C₆alkyl, at least one optionally substituted C₁-C₆alkyl is —CH₂CH₃.

In some embodiments wherein Ar is a substituted 5-10 membered heteroaryl group that is substituted with one or more optionally substituted C₁-C₆alkyl, at least one optionally substituted C₁-C₆alkyl is isopropyl.

In some embodiments wherein Ar is a substituted 5-10 membered heteroaryl group that is substituted with one or more optionally substituted C₁-C₆alkyl, at least one substituted C₁-C₆alkyl is —CH₂CF₃.

In some embodiments wherein Ar is a substituted 5-10 membered heteroaryl group that is substituted with one or more optionally substituted C₁-C₆alkyl, at least one substituted C₁-C₆alkyl is —CHF₂.

In some embodiments wherein Ar is a substituted 5-10 membered heteroaryl group that is substituted with one or more optionally substituted C₁-C₆alkyl, at least one substituted C₁-C₆alkyl is —CF₃.

In some embodiments wherein Ar is a substituted 5-10 membered heteroaryl group that is substituted with one or more optionally substituted C₁-C₆alkyl, at least one substituted C₁-C₆alkyl is —CH₂-cyclopropyl.

In some embodiments wherein Ar is a substituted 5-10 membered heteroaryl group that is substituted with one or more optionally substituted C₁-C₆alkyl, the optionally substituted C₁-C₆alkyl is —CH₂-(optionally substituted 5 or 6-membered heterocycloalkyl), such as, for example,

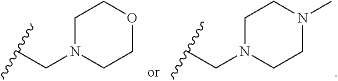

In some embodiments wherein Ar is a substituted 5-10 membered heteroaryl group that is substituted with one or more optionally substituted C₁-C₆alkyl, the optionally substituted C₁-C₆alkyl is —CH₂CH₂OH.

In some embodiments wherein Ar is a substituted 5-10 membered heteroaryl group that is substituted with one or more optionally substituted C₁-C₆alkyl, the optionally substituted C₁-C₆alkyl is —CH₂CH(OH)CH₃.

In some embodiments wherein Ar is a substituted 5-10 membered heteroaryl group that is substituted with one or more optionally substituted C₁-C₆alkyl, the optionally substituted C₁-C₆alkyl is —C(CN)(CH₃)₂.

In some embodiments wherein Ar is a substituted 5-10 membered heteroaryl group that is substituted with one or more optionally substituted C₁-C₆alkyl, the optionally substituted C₁-C₆alkyl is —CH₂CH₂CH₂OH.

In some embodiments wherein Ar is a substituted 5-10 membered heteroaryl group that is substituted with one or more optionally substituted C₁-C₆alkyl, the optionally substituted C₁-C₆alkyl is —CH₂CH₂OCH₃.

In some embodiments wherein Ar is a substituted 5-10 membered heteroaryl group that is substituted with one or more optionally substituted C₁-C₆alkyl, the optionally substituted C₁-C₆alkyl is —CH₂C(OH)(CH₃)₂.

In some embodiments wherein Ar is a substituted 5-10 membered heteroaryl group that is substituted with one or more optionally substituted C₁-C₆alkyl, the optionally substituted C₁-C₆alkyl is —CH₂CH₂OCH₂CH₃.

In some embodiments wherein Ar is a substituted 5-10 membered heteroaryl group that is substituted with one or more optionally substituted C₁-C₆alkyl, the optionally substituted C₁-C₆alkyl is —CH₂CH₂OCH(CH₃)₂.

In some embodiments wherein Ar is a substituted 5-10 membered heteroaryl group that is substituted with one or more optionally substituted C₁-C₆alkyl, the optionally substituted C₁-C₆alkyl is —CH₂CH(OCH₃)CH₃.

In some embodiments wherein Ar is a substituted 5-10 membered heteroaryl group that is substituted with one or more optionally substituted C₁-C₆alkyl, the optionally substituted C₁-C₆alkyl is —CH₂CHF₂.

In some embodiments wherein Ar is a substituted 5-10 membered heteroaryl group that is substituted with one or more optionally substituted C₁-C₆alkyl, the optionally substituted C₁-C₆alkyl is —CH₂CH₂N(CH₃)₂.

In some embodiments wherein Ar is a substituted 5-10 membered heteroaryl group that is substituted with one or more optionally substituted C₁-C₆alkyl, the optionally substituted C₁-C₆alkyl is —CH₂CH₂-(optionally substituted 5 or 6-membered heterocycloalkyl), such as, for example,

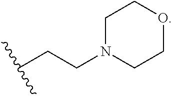 [SMILES: *CCN1CCOCC1]

In some embodiments wherein Ar is a substituted 5-10 membered heteroaryl group that is substituted with one or more optionally substituted C₁-C₆alkyl, the optionally substituted C₁-C₆alkyl is —CH₂-optionally substituted cyclopropyl, such as, for example,

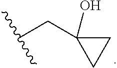 [SMILES: *CC1(O)CC1]

In some embodiments wherein Ar is a substituted 5-10 membered heteroaryl group that is substituted with one or more optionally substituted C₃-C₆cycloalkyl, the optionally substituted C₃-C₆cycloalkyl is cyclopropyl or cyclobutyl.

In some embodiments wherein Ar is a substituted 5-10 membered heteroaryl group that is substituted with one or more optionally substituted C₃-C₆cycloalkyl, the optionally substituted C₃-C₆cycloalkyl is cyclopropyl.

In some embodiments wherein Ar is a substituted 5-10 membered heteroaryl group that is substituted with one or more optionally substituted C₃-C₆cycloalkyl, the optionally substituted C₃-C₆cycloalkyl is cyclobutyl.

In some embodiments wherein Ar is a substituted 5-10 membered heteroaryl group that is substituted with one or more optionally substituted 4-6-membered heterocycloalkyl, the optionally substituted 4-6-membered heterocycloalkyl is

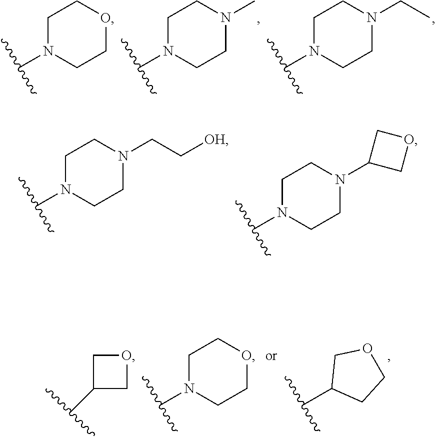

In some embodiments wherein Ar is a substituted 5-10 membered heteroaryl group that is substituted with one or more optionally substituted 4-6-membered heterocycloalkyl, the optionally substituted 4-6-membered heterocycloalkyl is

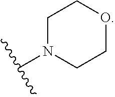 [SMILES: *N1CCOCC1]

In some embodiments wherein Ar is a substituted 5-10 membered heteroaryl group that is substituted with one or more optionally substituted 4-6-membered heterocycloalkyl, the optionally substituted 4-6-membered heterocycloalkyl is

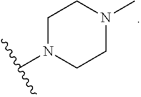 [SMILES: *N1CCN(C)CC1]

In some embodiments wherein Ar is a substituted 5-10 membered heteroaryl group that is substituted with one or more optionally substituted 4-6-membered heterocycloalkyl, the optionally substituted 4-6-membered heterocycloalkyl is

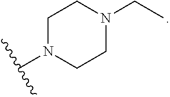 [SMILES: *N1CCN(CC)CC1]

In some embodiments wherein Ar is a substituted 5-10 membered heteroaryl group that is substituted with one or more optionally substituted 4-6-membered heterocycloalkyl, the optionally substituted 4-6-membered heterocycloalkyl is

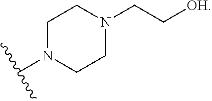 [SMILES: *N1CCN(CCO)CC1]

In some embodiments wherein Ar is a substituted 5-10 membered heteroaryl group that is substituted with one or more optionally substituted 4-6-membered heterocycloalkyl, the optionally substituted 4-6-membered heterocycloalkyl is

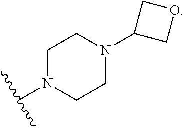 [SMILES: *N1CCN(C2COC2)CC1]

In some embodiments wherein Ar is a substituted 5-10 membered heteroaryl group that is substituted with one or more optionally substituted 4-6-membered heterocycloalkyl, the optionally substituted 4-6-membered heterocycloalkyl is

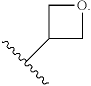 [SMILES: *C1COC1]

In some embodiments wherein Ar is a substituted 5-10 membered heteroaryl group that is substituted with one or more N(C₁-C₆alkyl)(optionally substituted C₁-C₆alkyl), the N(C₁-C₆alkyl)(optionally substituted C₁-C₆alkyl) is

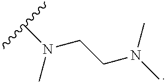 [SMILES: *N(C)CCN(C)C]

In some embodiments wherein Ar is a substituted 5-10 membered heteroaryl group that is substituted with one or more —O(optionally substituted C₁-C₆alkyl), the —O(optionally substituted C₁-C₆alkyl) is —OCH₃, —OCH₂CH₃, or

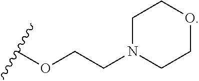 [SMILES: *OCCN1CCOCC1]

In some embodiments wherein Ar is a substituted 5-10 membered heteroaryl group that is substituted with one or more —O(optionally substituted C₁-C₆alkyl), the —O(optionally substituted C₁-C₆alkyl) is —OCH₃.

In some embodiments wherein Ar is a substituted 5-10 membered heteroaryl group that is substituted with one or more —O(optionally substituted C₁-C₆alkyl), the —O(optionally substituted C₁-C₆alkyl) is —OCH₂CH₃.

In some embodiments wherein Ar is a substituted 5-10 membered heteroaryl group that is substituted with one or more —O(optionally substituted C₁-C₆alkyl), the —O(optionally substituted C₁-C₆alkyl) is

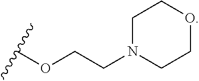 [SMILES: *OCCN1CCOCC1]

In some embodiments wherein Ar is a substituted 5-10 membered heteroaryl group, the phenyl group is substituted with —CN.

In some embodiments wherein Ar is a substituted 5-10 membered heteroaryl group that is substituted with one or more halogen, the halogen is —F.

In some embodiments, Ar in the compounds of formula I is

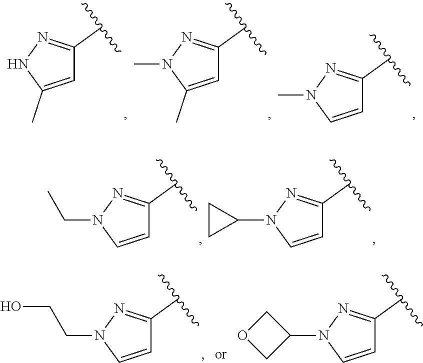

In some embodiments, Ar in the compounds of formula I is

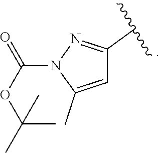 [SMILES: *c1cc(C)n(C(=O)OC(C)(C)C)n1]

In other embodiments, Ar in the compounds of formula I is

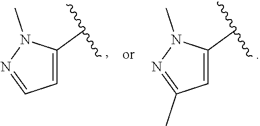

In other embodiments, Ar in the compounds of formula I is

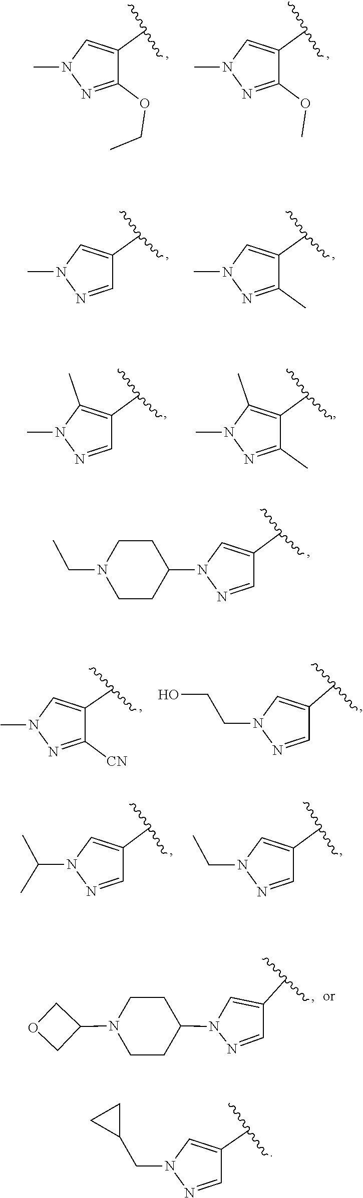

In other embodiments, Ar in the compounds of formula I is

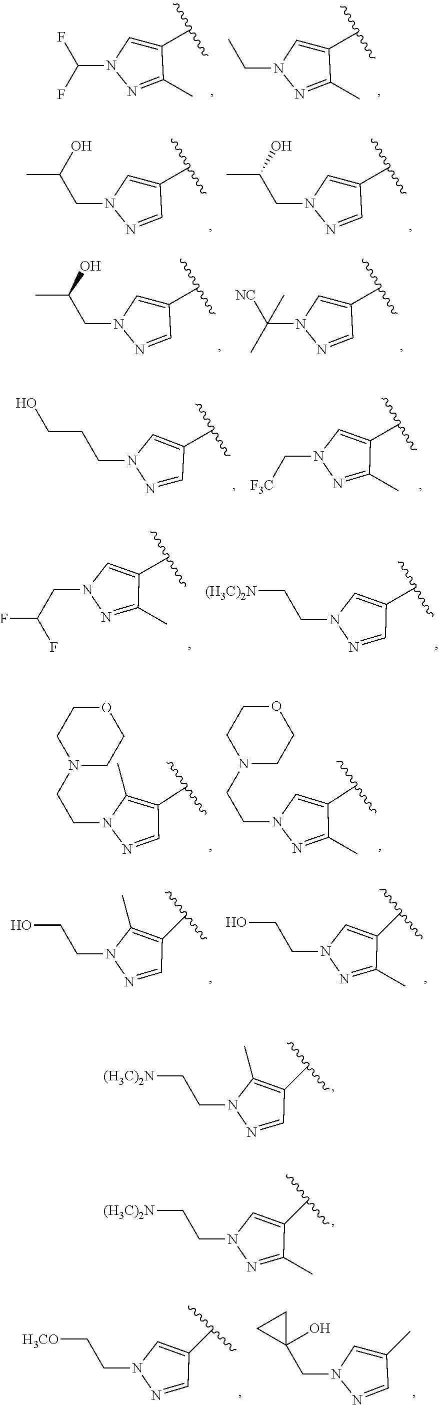

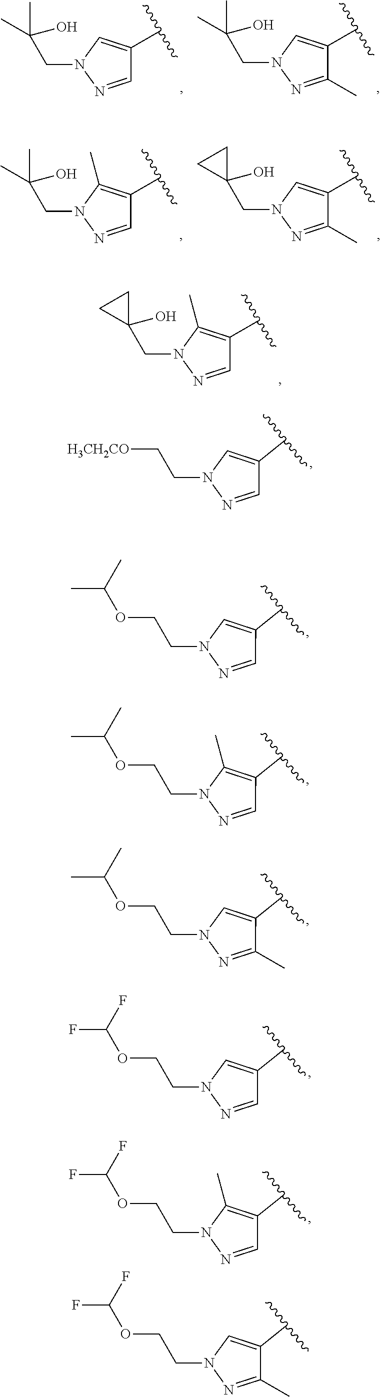

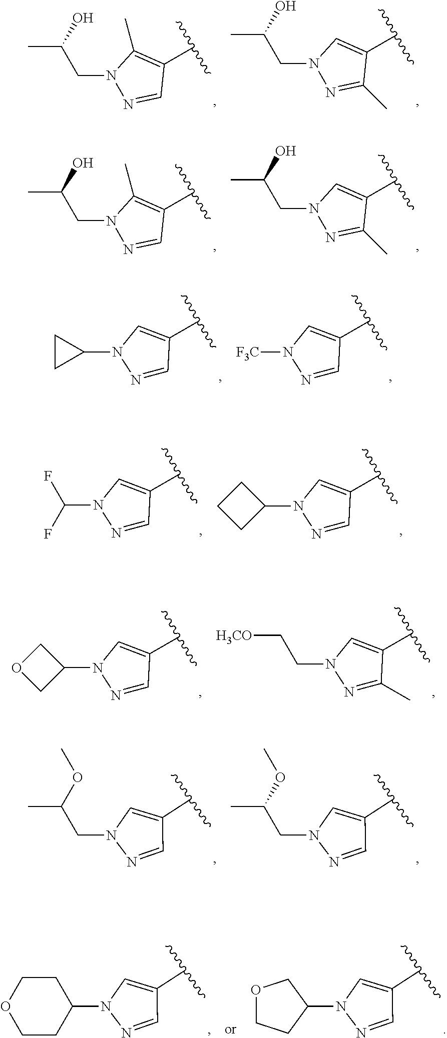

In other embodiments, Ar in the compounds of formula I is

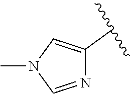 [SMILES: *c1cn(C)cn1]

In other embodiments, Ar in the compounds of formula I is

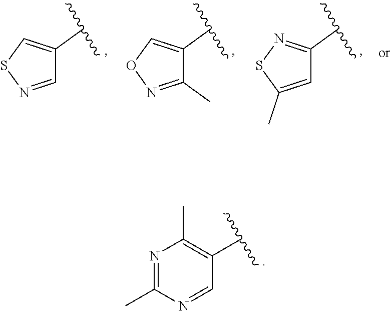

In other embodiments, Ar in the compounds of formula I is

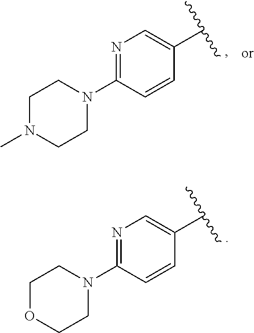 [SMILES: *c1ccc(N2CCN(C)CC2)nc1 ; *c1ccc(N2CCOCC2)nc1]

In other embodiments, Ar in the compounds of formula I is

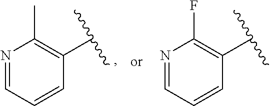

In other embodiments, Ar in the compounds of formula I is

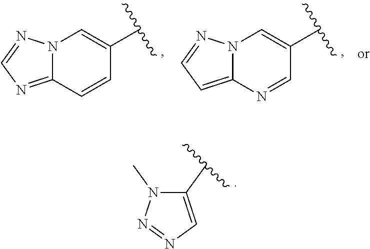

In other embodiments, Ar in the compounds of formula I is

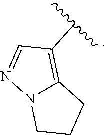 [SMILES: *c1cnn2c1CCC2]

In some aspects, R¹ in formula I is H, F, Cl, Br, C₁₋₆alkyl, CN, C₁₋₆haloalkyl, —C(O)NH₂, —C(O)NH(C₁₋₆alkyl), or —C(O)N(C₁₋₆alkyl)₂.

In some embodiments, R¹ is H, —Cl, —F, —CH₃, or —CN.

In some embodiments, R¹ is —C₁ or —CH₃.

In some embodiments, R¹ is H.

In some embodiments, R¹ is F, with the proviso that when R¹ is F, Ar is not

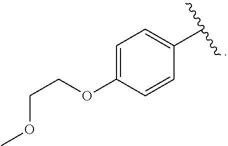 [SMILES: *c1ccc(OCCOC)cc1]

In some embodiments, R¹ is C₁. In some embodiments, R¹ is Br.

In some embodiments, R¹ is C₁₋₆alkyl, such as, for example, C₆alkyl, C₅alkyl, C₄alkyl, C₃alkyl, C₂alkyl, C₁alkyl, methyl, ethyl, propyl, and the like.

In some embodiments, R¹ is —CH₃.

In some embodiments, R¹ is CN.

In some embodiments, R¹ is C₁₋₆haloalkyl, such as, for example, C₆haloalkyl, C₅haloalkyl, C₄haloalkyl, C₃haloalkyl, C₂haloalkyl, C₁haloalkyl, —CF₃, —CHF₂, —CH₂CF₃, and the like.

In some embodiments, R¹ is —C(O)NH₂.

In some embodiments, R¹ is —C(O)NH(C₁₋₆alkyl), such as, for example, —C(O)NH(C₆alkyl), —C(O)NH(C₅alkyl), —C(O)NH(C₄alkyl), —C(O)NH(C₃alkyl), —C(O)NH(C₂alkyl), —C(O)NH(C₁alkyl), —C(O)NH(CH₃), —C(O)NH(CH₂CH₃), —C(O)NH(CH₂CH₂CH₃), and the like.

In some embodiments, R¹ is —C(O)N(C₁₋₆alkyl)₂, wherein each C₁₋₆alkyl can be the same or different, such as, for example, —C(O)N(C₆alkyl)₂, —C(O)N(C₅alkyl)₂, —C(O)N(C₄alkyl)₂, —C(O)N(C₃alkyl)₂, —C(O)N(C₂alkyl)₂, —C(O)N(C₁alkyl)₂, —C(O)N(CH₃)₂, —C(O)N(CH₂CH₃)₂, —C(O)N(CH₂CH₃)(CH₃), and the like.

In some aspects, R² in formula I is a 9-membered bicyclic heteroaryl comprising a 6-5-fused ring system wherein the 5-membered ring contains at least one nitrogen atom and the 6 membered ring is substituted with at least one E, wherein E is an electrophilic moiety that is capable of reacting with a nucleophile so as to form a covalent bond between an atom of the nucleophile and an atom of the electrophilic moiety. Exemplary E groups are described herein.

In these embodiments of R², the 9-membered bicyclic heteroaryl comprising a 6-5-fused ring system is any fully aromatic 6-5 fused ring system in which the 5-membered ring contains at least one nitrogen atom. The 5-membered ring may also contain, in addition to the nitrogen, one more additional heteroatoms that may be another nitrogen, an oxygen, or a sulfur. The 6-membered ring may contain one or more nitrogen atoms.

In other aspects, R² in formula I is a 13-membered tricyclic heteroaryl comprising a 6-5-6-fused ring system wherein the 5-membered ring contains at least one nitrogen atom and one of the 6 membered rings is substituted with at least one E, wherein E is an electrophilic moiety that is capable of reacting with a nucleophile so as to form a covalent bond between an atom of the nucleophile and an atom of the electrophilic moiety.

In these embodiments of R², the 13-membered tricyclic heteroaryl comprising a 6-5-6-fused ring system is any fully aromatic 6-5-6 fused ring system in which the 5-membered ring contains at least one nitrogen atom. The 5-membered ring may also contain, in addition to the nitrogen, one more additional heteroatoms that may be another nitrogen, an oxygen, or a sulfur. The 6-membered rings may contain one or more nitrogen atoms.

In other aspects, R² in formula I is a 12- or 13-membered tricyclic group comprising a 5- or 6-membered cycloalkyl or heterocycloalkyl ring fused to a 9-membered bicyclic heteroaryl comprising a 6-5-fused ring system; wherein the 5-membered ring contains at least one nitrogen atom and the 6 membered ring is substituted with at least one E; wherein E is an electrophilic moiety that is capable of reacting with a nucleophile so as to form a covalent bond between an atom of the nucleophile and an atom of the electrophilic moiety.

In these embodiments of R², the 9-membered bicyclic heteroaryl comprising a 6-5-fused ring system is any fully aromatic 6-5 fused ring system in which the 5-membered ring contains at least one nitrogen atom. The 5-membered ring may also contain, in addition to the nitrogen, one more additional heteroatoms that may be another nitrogen, an oxygen, or a sulfur. The 6-membered ring may contain one or more nitrogen atoms. The 5- or 6-membered cycloalkyl or heterocycloalkyl ring is fused to the 5-membered ring of the 6-5 ring system.

In some embodiments of formula I, R² is

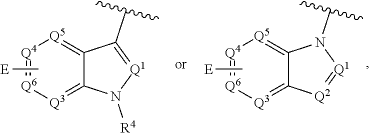

wherein
Q¹ is N or C—R^{3A};
Q² is N or C—R^{3A},
   R^{3A} is H, C₁-C₆alkyl, —CN, —C₁-C₆haloalkyl, halo, —CON(R)₂, —NR₂, cycloalkyl, —CH₂cycloalkyl, —COR, —CH(OH)R, —CO-cycloalkyl, —CH(OH)cycloalkyl, —SR, or —SO₂R;
      each R is independently H, or C₁-C₆alkyl;
   or, when Q¹ is C—R^{3A} and Q² is C—R^{3A}, the R^{3A} of Q¹ and the R^{3A} of Q², together with the carbon atoms to which they are attached, for a 5- or 6-membered cycloalkyl or heterocycloalkyl ring, or a phenyl ring;
Q³, Q⁴, Q⁵, and Q⁶ are each independently N or C—R^{3B}; each R^{3B} is independently H, —C₁-C₆alkyl, —C₁-C₆haloalkyl, —CN, C₃-C₇cycloalkyl, or E, provided that at least one of Q³, Q⁴, Q⁵, and Q⁶ is C-E, wherein E is an electrophilic moiety that is capable of reacting with a nucleophile so as to form a covalent bond between an atom of the nucleophile and an atom of the electrophilic moiety; and
R⁴ is C₁-C₆alkyl, C₁-C₆haloalkyl, C₃-C₇cycloalkyl, or SO₂(C₁-C₆alkyl); or, when Q¹ is C—R^{3A}, the R^{3A} of Q¹ and R⁴, together with the atoms to which they are attached, form a 5- or 6-membered heterocycloalkyl ring.

In some embodiments of formula I, R² is

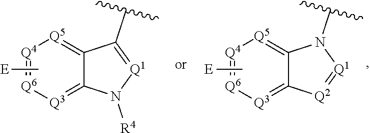

Q¹ is N or C—H;
Q² is N or C—R^{3A},
   R^{3A} is H, C₁-C₆alkyl, —CN, —C₁-C₆haloalkyl, halo, —CON(R)₂, —NR₂, cycloalkyl, —CH₂cycloalkyl, —COR, —CH(OH)R, —CO-cycloalkyl, —CH(OH)cycloalkyl, —SR, or —SO₂R;
      each R is independently H, or C₁-C₆alkyl;
Q³, Q⁴, Q⁵, and Q⁶ are each independently N or C—R^{3B};
   each R^{3B} is independently H, —C₁-C₆alkyl, —C₁-C₆haloalkyl, —CN, C₃-C₇cycloalkyl, or E, provided that at least one of Q³, Q⁴, Q⁵, and Q⁶ is C-E, wherein E is an electrophilic moiety that is capable of reacting with a nucleophile so as to form a covalent bond between an atom of the nucleophile and an atom of the electrophilic moiety; and
R⁴ is C₁-C₆alkyl, C₁-C₆haloalkyl, C₃-C₇cycloalkyl, or SO₂(C₁-C₆alkyl).

In some embodiments of formula I, R² is

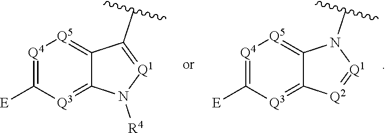

In some embodiments of R², Q¹ is N.

In other embodiments of R², Q¹ is C—R^{3A}. In such embodiments, R^{3A} is H, C₁-C₆alkyl, —CN, —C₁-C₆haloalkyl, halo, —CON(R)₂, —NR₂, cycloalkyl, —CH₂cycloalkyl, —COR, —CH(OH)R, —CO-cycloalkyl, —CH(OH)cycloalkyl, —SR, or —SO₂R, wherein each R is independently H, or C₁-C₆alkyl.

In other embodiments of R², Q¹ is C—H.

In other embodiments of R², Q¹ is C—CH₃.

In some embodiments of R², Q² is N.

In other embodiments of R², Q² is C—R^{3A}.

In some embodiments, R^{3A} is H, C₁-C₆alkyl, —CN, —C₁-C₆haloalkyl, halo, —CON(R)₂, —NR₂, cycloalkyl, —CH₂cycloalkyl, —COR, —CH(OH)R, —CO-cycloalkyl, —CH(OH)cycloalkyl, —SR, or —SO₂R, wherein each R is independently H, or C₁-C₆alkyl.

In some embodiments, R^{3A} is H.

In other embodiments, R^{3A} is C₁-C₆alkyl, such as, for example, C₆alkyl, C₅alkyl, C₄alkyl, C₃alkyl, C₂alkyl, C₁alkyl, methyl, ethyl, propyl, and the like.

In some embodiments, R^{3A} is methyl.

In some embodiments, R^{3A} is ethyl.

In some embodiments, R^{3A} is —CN.

In other embodiments, R^{3A} is C₁₋₆haloalkyl, such as, for example, C₆haloalkyl, C₅haloalkyl, C₄haloalkyl, C₃haloalkyl, C₂haloalkyl, C₁haloalkyl, —CF₃, —CHF₂, —CH₂CF₃, and the like.

In some embodiments, R^{3A} is halo, i.e., —F, —Cl, Br, or —I.

In some embodiments, R^{3A} is —CON(R)₂, wherein each R is independently H, or C₁-C₆alkyl (such as, for example, C₆alkyl, C₅alkyl, C₄alkyl, C₃alkyl, C₂alkyl, C₁alkyl, methyl, ethyl, propyl, and the like.). Thus, in some embodiments wherein R^{3A} is —CON(R)₂, R^{3A} is, for example, —CONH₂, —CON(CH₃)₂, —CON(CH₂CH₃)₂, —CONH(CH₃), —CONH(CH₂CH₃), —CON(CH₃)(CH₂CH₃), and the like.

In some embodiments, R^{3A} is —NR₂, wherein each R is independently H, or C₁-C₆alkyl (such as, for example, C₆alkyl, C₅alkyl, C₄alkyl, C₃alkyl, C₂alkyl, C₁alkyl, methyl, ethyl, propyl, and the like.). Thus, in some embodiments wherein R^{3A} is —NR₂, R^{3A} is, for example, —NH₂, —N(CH₃)₂, —N(CH₂CH₃)₂, —NH(CH₃), —NH(CH₂CH₃), —N(CH₃)(CH₂CH₃), and the like.

In some embodiments, R^{3A} is -cycloalkyl, such as, for example, cyclopropyl, cyclobutyl, cyclopentyl, cyclohexyl, and the like.

In some embodiments, R^{3A} is —CH₂cycloalkyl, such as, for example, —CH₂cyclopropyl, —CH₂cyclobutyl, —CH₂cyclopentyl, —CH₂cyclohexyl, and the like.

In some embodiments, R^{3A} is —COR, wherein R is independently H, or C₁-C₆alkyl (such as, for example, C₆alkyl, C₅alkyl, C₄alkyl, C₃alkyl, C₂alkyl, C₁alkyl, methyl, ethyl, propyl, and the like.). Thus, in some embodiments wherein R^{3A} is —COR, R^{3A} is, for example, —COH, —COCH₃, —COCH₂CH₃, and the like.

In some embodiments, R^{3A} is —CH(OH)R, wherein R is independently H, or C₁-C₆alkyl (such as, for example, C₆alkyl, C₅alkyl, C₄alkyl, C₃alkyl, C₂alkyl, C₁alkyl, methyl, ethyl, propyl, and the like.). Thus, in some embodiments wherein R^{3A} is —CH(OH)R, R^{3A} is, for example, —CH₂(OH), —CH(OH)CH₃, —CH(OH)CH₂CH₃, and the like.

In some embodiments, R^{3A} is —CO-cycloalkyl. Thus, in some embodiments wherein R^{3A} is —CO-cycloalkyl, R^{3A} is, for example, —CO-cyclopropyl, —CO-cyclobutyl, —CO— cyclopentyl, —CO-cyclohexyl, and the like.

In some embodiments, R^{3A} is —CH(OH)-cycloalkyl. Thus, in some embodiments wherein R^{3A} is —CH(OH)-cycloalkyl, R^{3A} is, for example, —CH(OH)-cyclopropyl, —CH(OH)— cyclobutyl, —CH(OH)-cyclopentyl, —CH(OH)-cyclohexyl, and the like.

In some embodiments, R^{3A} is —SR wherein each R is independently H, or C₁-C₆alkyl (such as, for example, C₆alkyl, C₅alkyl, C₄alkyl, C₃alkyl, C₂alkyl, C₁alkyl, methyl, ethyl, propyl, and the like.). Thus, in some embodiments wherein R^{3A} is —SR, R^{3A} is, for example, —SH, —SCH₃, —SCH₂CH₃, —SCH₂CH₂CH₃, and the like.

In some embodiments, R^{3A} is —SO₂R wherein each R is independently H, or C₁-C₆alkyl (such as, for example, C₆alkyl, C₅alkyl, C₄alkyl, C₃alkyl, C₂alkyl, C₁alkyl, methyl, ethyl, propyl, and the like.). Thus, in some embodiments wherein R^{3A} is —SO₂R, R^{3A} is, for example, —SO₂H, —SO₂CH₃, —SO₂CH₂CH₃, —SO₂CH₂CH₂CH₃, and the like.

In some embodiments of R², Q³ is N or C—R^{3B}.

In some embodiments wherein Q¹ is C—R^{3A} and Q² is C—R^{3A}, the R^{3A} of Q¹ and the R^{3A} of Q² are both —CH₃.

In some embodiments wherein Q¹ is C—R^{3A} and Q² is C—R^{3A}, the R^{3A} of Q¹ and the R^{3A} of Q², together with the carbon atoms to which they are attached, form a 5- or 6-membered cycloalkyl or heterocycloalkyl ring; or a 6-membered phenyl ring.

In some embodiments wherein Q¹ is C—R^{3A} and Q² is C—R^{3A}, the R^{3A} of Q¹ and the R^{3A} of Q², together with the carbon atoms to which they are attached, form a 5-membered cycloalkyl ring. Thus, in some embodiments, R² is

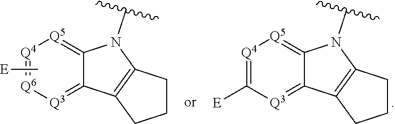

In other embodiments wherein Q¹ is C—R^{3A} and Q² is C—R^{3A}, the R^{3A} of Q¹ and the R^{3A} of Q², together with the carbon atoms to which they are attached, form a 6-membered cycloalkyl ring. Thus, in some embodiments, R² is

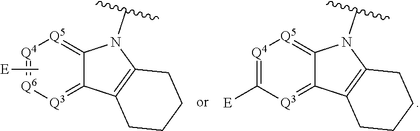

In other embodiments wherein Q¹ is C—R^{3A} and Q² is C—R^{3A}, the R^{3A} of Q¹ and the R^{3A} of Q², together with the carbon atoms to which they are attached, form a phenyl ring. Thus, in some embodiments, R² is

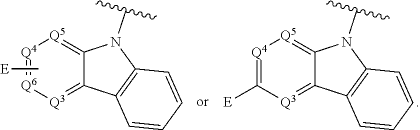

In some embodiments Q³ is N.

In some embodiments Q³ is C—R^{3B}.

In some embodiments Q⁴ is N.

In some embodiments Q⁴ is C—R^{3B}.

In some embodiments Q⁵ is N.

In some embodiments Q⁵ is C—R^{3B}.

In some embodiments Q⁶ is N.

In some embodiments Q⁶ is C—R^{3B}.

In embodiments in which Q³, Q⁴, Q⁵, and/or Q⁶ is C—R^{3B}, each R^{3B} is independently H, —C₁-C₆alkyl, —C₁-C₆haloalkyl, —CN, C₃-C₇cycloalkyl, or E.

In some embodiments, R^{3B} is H.

In other embodiments, R^{3B} is C₁-C₆alkyl, such as, for example, C₆alkyl, C₅alkyl, C₄alkyl, C₃alkyl, C₂alkyl, C₁alkyl, methyl, ethyl, propyl, and the like.

In some embodiments, R^{3B} is methyl.

In some embodiments, R^{3B} is —CN.

In other embodiments, R^{3B} IS C₁₋₆haloalkyl, such as, for example, C₆haloalkyl, C₅haloalkyl, C₄haloalkyl, C₃haloalkyl, C₂haloalkyl, C₁haloalkyl, —CF₃, —CHF₂, —CH₂CF₃, and the like.

In some embodiments, R^{3B} is —C₃-C₇cycloalkyl, such as, for example, —C₃cycloalkyl, —C₄cycloalkyl, —C₅cycloalkyl, —C₆cycloalkyl, —C₇cycloalkyl, cyclopropyl, cyclobutyl, cyclopentyl, cyclohexyl, and the like.

In some embodiments, R^{3B} is E wherein E is an electrophilic moiety that is capable of reacting with a nucleophile so as to form a covalent bond between an atom of the nucleophile and an atom of the electrophilic moiety. Exemplary E groups are further described herein.

In some embodiments, R⁴ is C₁-C₆alkyl, C₁-C₆haloalkyl, C₃-C₇cycloalkyl, or SO₂(C₁-C₆alkyl).

In some embodiments, R⁴ is C₁-C₆alkyl, such as, for example, C₆alkyl, C₅alkyl, C₄alkyl, C₃alkyl, C₂alkyl, C₁alkyl, methyl, ethyl, propyl, and the like.

In some embodiments, R⁴ is C₁₋₆haloalkyl, such as, for example, C₆haloalkyl, C₅haloalkyl, C₄haloalkyl, C₃haloalkyl, C₂haloalkyl, C₁haloalkyl, —CF₃, —CHF₂, —CH₂CF₃, and the like.

In some embodiments, R⁴ is —C₃-C₇cycloalkyl, such as, for example, —C₃cycloalkyl, —C₄cycloalkyl, —C₅cycloalkyl, —C₆cycloalkyl, —C₇cycloalkyl, cyclopropyl, cyclobutyl, cyclopentyl, cyclohexyl, and the like.

In some embodiments, R⁴ is —SO₂(C₁-C₆alkyl), such as, for example, —SO₂(C₁alkyl), —SO₂(C₂alkyl), —SO₂(C₃alkyl), —SO₂(C₄alkyl), —SO₂(C₅alkyl), —SO₂(C₆alkyl), —SO₂(CH₃), —SO₂(CH₂CH₃), —SO₂(CH₂CH₂CH₃), and the like.

In some embodiments wherein Q¹ is C—R^{3A}, the R^{3A} of Q¹ and R⁴, together with the atoms to which they are attached, form a 5- or 6-membered heterocycloalkyl ring.

In some embodiments wherein Q¹ is C—R^{3A}, the R^{3A} of Q¹ and R⁴, together with the atoms to which they are attached, form a 5-membered heterocycloalkyl ring. Thus, in some embodiments, R² is

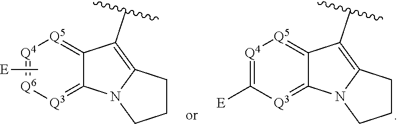

In some embodiments wherein Q¹ is C—R^{3A}, the R^{3A} of Q¹ and R⁴, together with the atoms to which they are attached, form a 6-membered heterocycloalkyl ring. Thus, in some embodiments, R² is

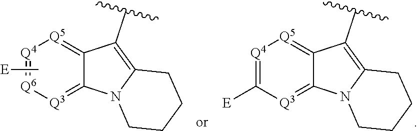

In some embodiments wherein Q³ is C—R^{3B}, R^{3B} is —CH₃.

In some embodiments wherein Q⁵ is C—R^{3B}, R^{3B} is —CH₃.

In some embodiments, R² is

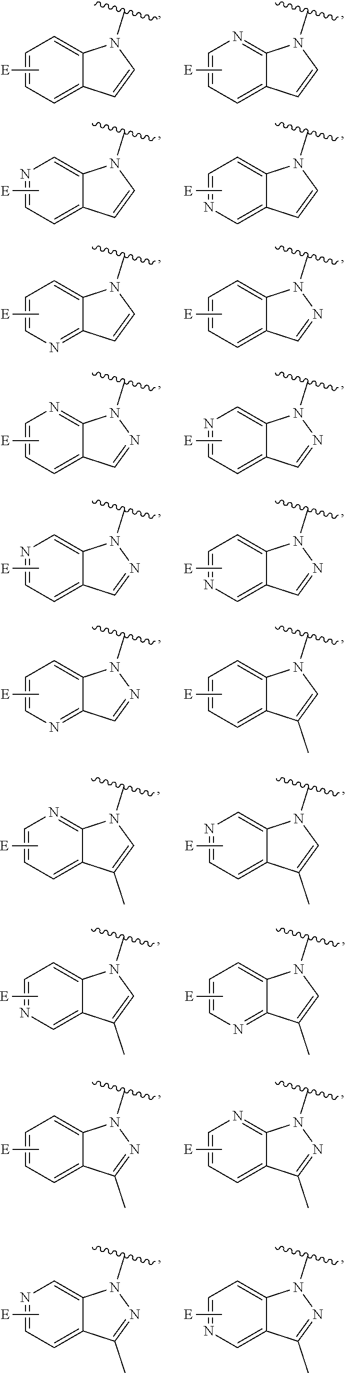

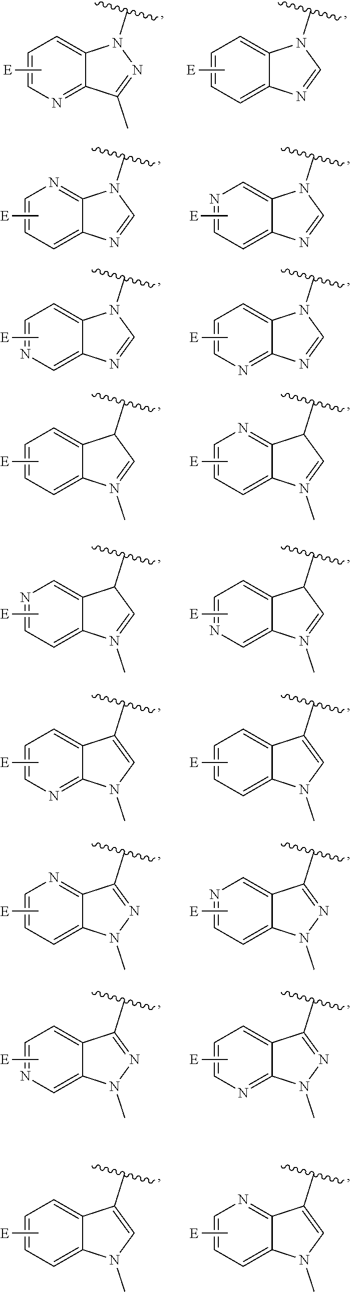

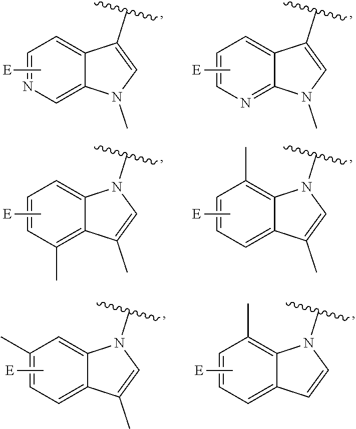

and the like.

In some embodiments, R² is

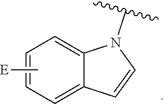

In some embodiments, R² is

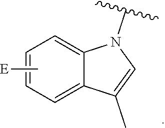

In some embodiments, R² is

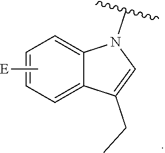

In some embodiments, R² is

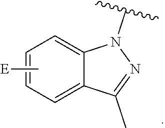

In some embodiments, R² is

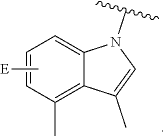

In some embodiments, R² is

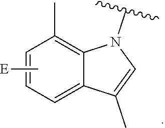

In some embodiments, R² is

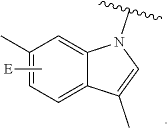

In some embodiments, R² is

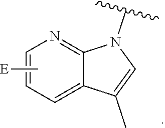

In some embodiments, R² is

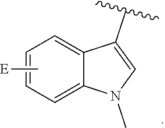

In some embodiments, R² is

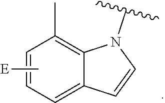

In some embodiments, R² is

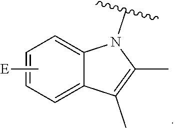

In some embodiments, R² is

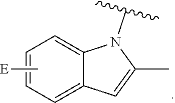

In some embodiments, R² is

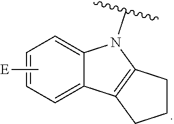

In some embodiments, R² is

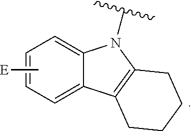

In some embodiments, R² is

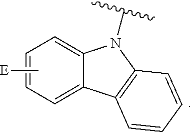

In some aspects, E in the compounds of the disclosure is an electrophilic moiety that is capable of reacting with a nucleophile so as to form a covalent bond between an atom of the nucleophile and an atom of the electrophilic moiety. Such electrophilic moieties include, but are not limited to, α-β unsaturated amides, α-β unsaturated esters, α-β unsaturated ketones, α-β unsaturated sulfoxides, α-β unsaturated sulfones, α-β unsaturated sulfinates, α-β unsaturated sulfonates, α-β unsaturated sulfinamides, α-β unsaturated sulfonamides, α-β unsaturated succinimides, α-β epoxy amides, α-β epoxy esters, α-β epoxy ketones, α-halo amides, α-halo esters, α-halo ketones, and the like.

In some embodiments, E is

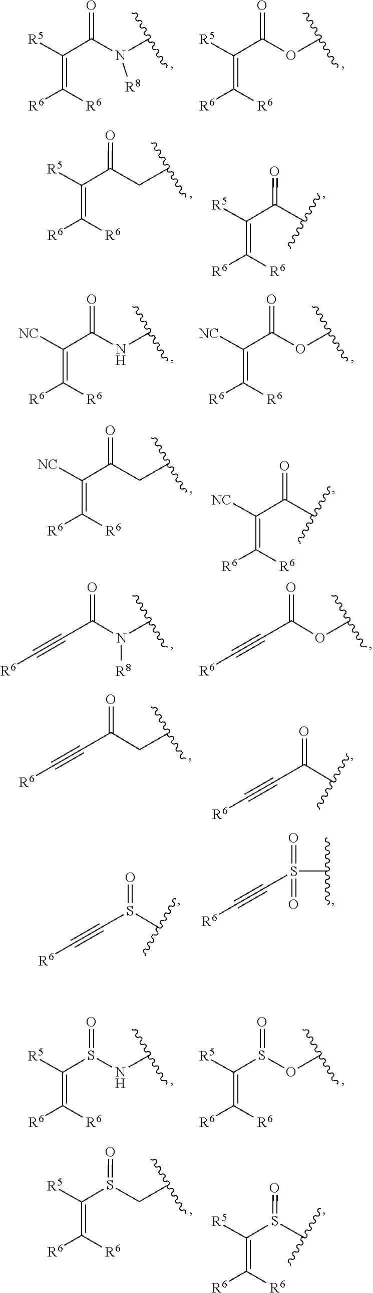

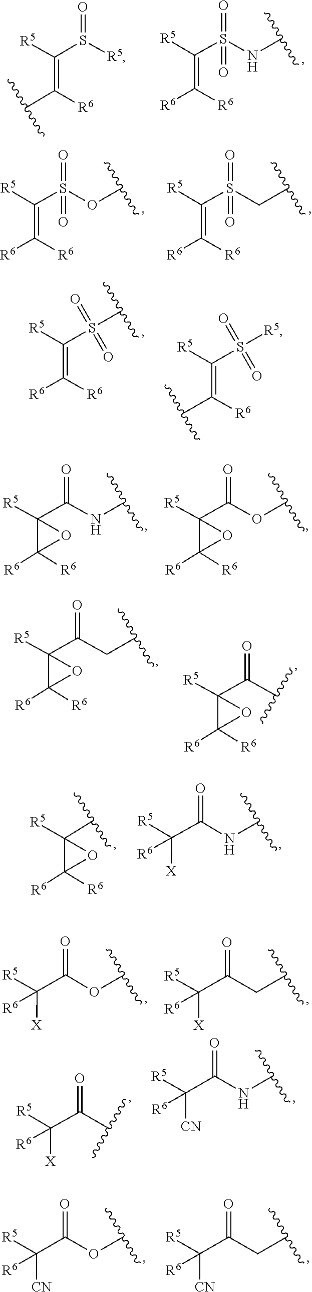

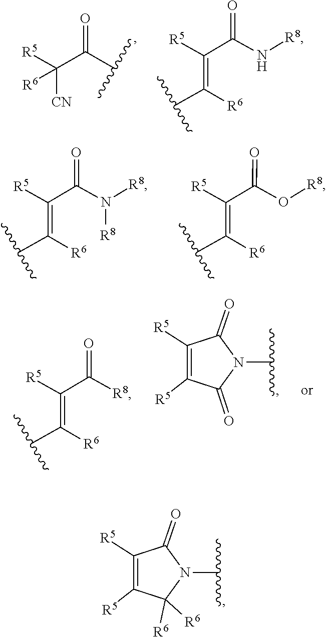

wherein each R⁵ and each R⁶ is independently H, optionally substituted C₁-C₆alkyl, or halogen; R⁸ is H or C₁-C₆alkyl; and X is halogen.

In some embodiments, R⁵ is H.

In other embodiments, R⁵ is optionally substituted C₁-C₆alkyl, such as, for example, optionally substituted C₆alkyl, C₅alkyl, C₄alkyl, C₃alkyl, C₂alkyl, C₁alkyl, methyl, ethyl, propyl, and the like.

In other embodiments, R⁵ is halogen, i.e., —F, —Cl, —Br, or —I.

In some embodiments, R⁶ is H.

In other embodiments, R⁶ is optionally substituted C₁-C₆alkyl, such as, for example, optionally substituted C₆alkyl, C₅alkyl, C₄alkyl, C₃alkyl, C₂alkyl, C₁alkyl, methyl, ethyl, propyl, and the like.

In other embodiments, R⁶ is halogen, i.e., —F, —Cl, —Br, or —I.

In some embodiments, R⁸ is H.

In other embodiments, R⁸ is C₁-C₆alkyl, such as, for example, C₆alkyl, C₅alkyl, C₄alkyl, C₃alkyl, C₂alkyl, C₁alkyl, methyl, ethyl, propyl, and the like.

In some embodiments, X is halogen, i.e., —F, —Cl, —Br, or —I.

In some embodiments of the compounds of the disclosure, E is

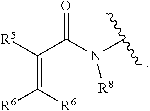

In some embodiments of the compounds of the disclosure, E is

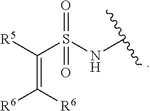

In some embodiments of the compounds of the disclosure, E is

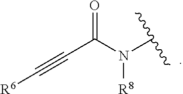

In some embodiments of the compounds of the disclosure, E is

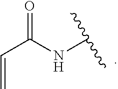 [SMILES: *NC(=O)C=C]

In some embodiments of the compounds of the disclosure, E is

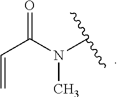 [SMILES: *N(C)C(=O)C=C]

In some embodiments of the compounds of the disclosure, E is

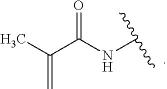 [SMILES: *NC(=O)C(=C)C]

In some embodiments of the compounds of the disclosure, E is

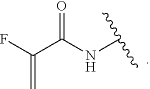 [SMILES: *NC(=O)C(=C)F]

In some embodiments of the compounds of the disclosure, E is

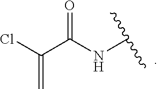 [SMILES: *NC(=O)C(=C)Cl]

In some embodiments of the compounds of the disclosure, E is

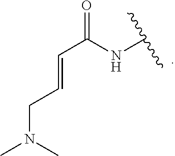 [SMILES: *NC(=O)/C=C/CN(C)C]

In some embodiments of the compounds of the disclosure, E is

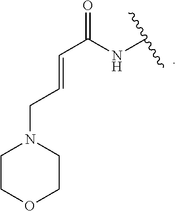 [SMILES: *NC(=O)/C=C/CN1CCOCC1]

In some embodiments of the compounds of the disclosure, E is

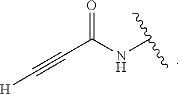 [SMILES: *NC(=O)C#C]

In some embodiments of the compounds of the disclosure, E is

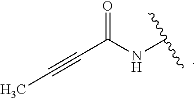 [SMILES: *NC(=O)C#CC]

In some embodiments of the compounds of the disclosure, E is

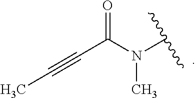 [SMILES: *N(C)C(=O)C#CC]

In some embodiments of the compounds of the disclosure, E is

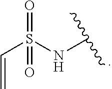 [SMILES: *NS(=O)(=O)C=C]

In some embodiments of the disclosure, the compounds of formula I are compounds of formula IA:

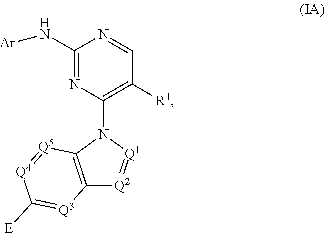

or pharmaceutically acceptable salts thereof, wherein Ar, R¹, Q¹; Q², Q³, Q⁴, Q⁵, and E have the definitions described above for formula I.

In some embodiments of the compounds of formula IA, Q¹ is C—H; Q² is C—R^{3A}; Q³ and Q⁴ are each C—R^{3B}; Q⁵ is N or C—R^{3B}; R^{3A} is H or —CH₃; and each R^{3B} is independently H or —CH₃.

In some embodiments of the compounds of formula IA, Q¹ is C—H; Q² is C—R^{3A}; Q³ and Q⁴ are each C—R^{3B}; Q⁵ is C—R^{3B}; R^{3A}—CH₃; and each R^{3B} is H.

In other embodiments of the compounds of formula IA, Q¹ is N; Q² is C—R^{3A}; Q³ and Q⁴ are each C—R^{3B}; Q⁵ is N or C—R^{3B}; R^{3A} is H or —CH₃; and each R^{3B} is independently H or —CH₃.

In some embodiments, the compounds of formula IA are compounds of formula IA-1:

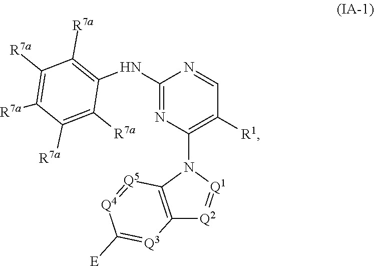

wherein each R^{7a} is independently H, —CH₃, —CH₂CH₃, isopropyl, —CH₂CF₃—CHF₂, —CF₃, —CH₂-cyclopropyl, —CH₂CH₂OH,

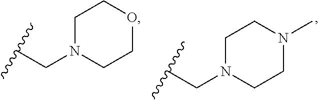

cyclopropyl, cyclobutyl,

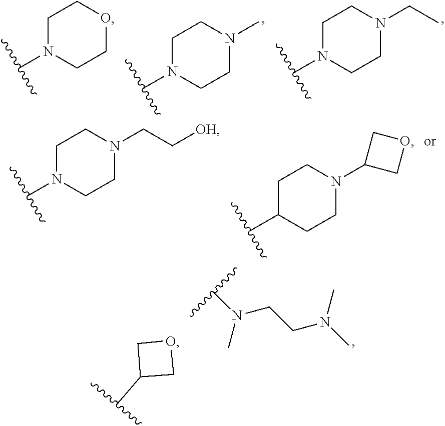

—OCH₃, —OCH₂CH₃,

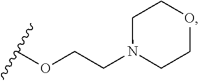 [SMILES: *OCCN1CCOCC1]

### —CN, or —F.

In some embodiments of the compounds of formula IA-1, Q¹ is C—H; Q² is C—R^{3A}; Q³ and Q⁴ are each C—R^{3B}; Q⁵ is N or C—R^{3B}; R^{3A} is H or —CH₃; and each R^{3B} is independently H or —CH₃.

In some embodiments of the compounds of formula IA-1, Q¹ is C—H; Q² is C—R^{3A}; Q³ and Q⁴ are each C—R^{3B}. Q⁵ is C—R^{3B}; R^{3A}—CH₃; and each R^{3B} is H.

In other embodiments of the compounds of formula IA-1, Q¹ is N; Q² is C—R^{3A}; Q³ and Q⁴ are each C—R^{3B}; Q⁵ is N or C—R^{3B}; R^{3A} is H or —CH₃; and each R^{3B} is independently H or —CH₃.

In some embodiments of the compounds of formula IA-1, four of the R^{7a} are H, and the other R^{7a} is —CH₃, —CH₂CH₃, isopropyl, —CH₂CF₃, —CHF₂, —CF₃, —CH₂-cyclopropyl, —CH₂CH₂OH,

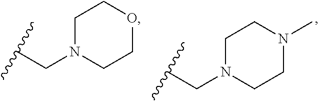

cyclopropyl, cyclobutyl,

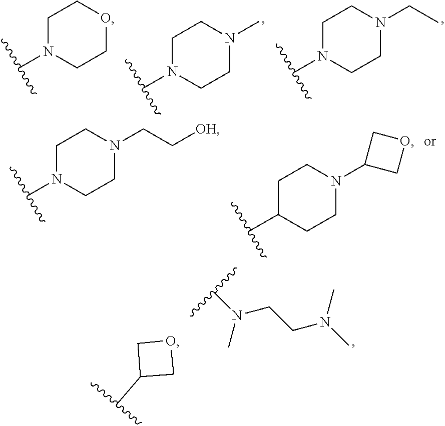

—OCH₃, —OCH₂CH₃,

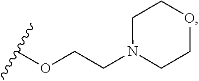 [SMILES: *OCCN1CCOCC1]

### or —F.

In some embodiments of the compounds of formula IA-1, three or four of the R^{7a} are H, and the other R^{7a} are —CH₃,

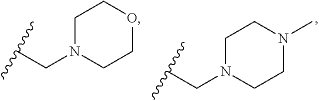

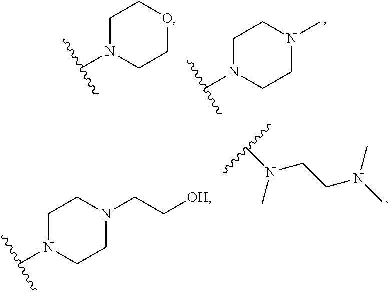

—OCH₃, —OCH₂CH₃,

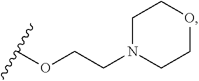 [SMILES: *OCCN1CCOCC1]

### or —F.

In some embodiments, the compounds of formula IA are compounds of formula IA-2:

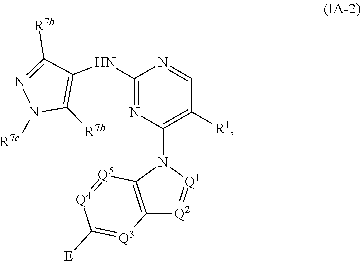

wherein each R^{7b} is independently H, —CH₃—CH₂CH₃, isopropyl, —CH₂CF₃, —CHF₂, —CF₃, —CH₂-cyclopropyl, —CH₂CH₂OH,

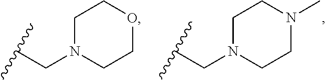

cyclopropyl, cyclobutyl,

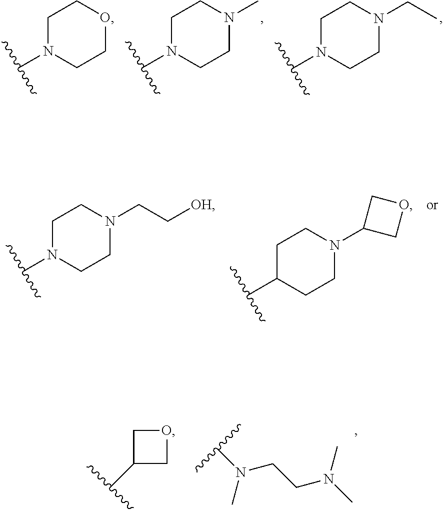

—OCH₃, —OCH₂CH₃,

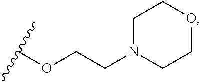 [SMILES: *OCCN1CCOCC1]

—CN, or —F; and R^{7c} is H, —CH₃, —CH₂CH₃, isopropyl, —CH₂CF₃, —CHF₂, —CF₃, —CH₂-cyclopropyl, —CH₂CH₂OH,

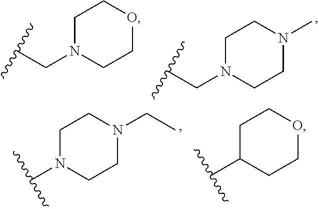

cyclopropyl, cyclobutyl,

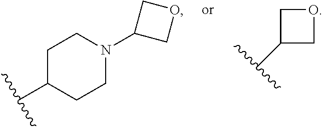 [SMILES: *C1CCN(C2COC2)CC1 ; *C1COC1]

In some embodiments of the compounds of formula IA-2, R^{7c} is —CH₂CHF₂; —CH₂—CH(OH)—CH₃, —CH₂C(OH)(CH₃)₂, —CH₂CH₂CH₂OH, —C(CH₃)₂—CN, —CH₂CH₂OCH₃, —CH₂CH₂OCH₂CH₃, —CH₂CH₂OCH(CH₃)₂, —CH₂CH₂OCHF₂, —CH₂CH₂N(CH₃)₂,

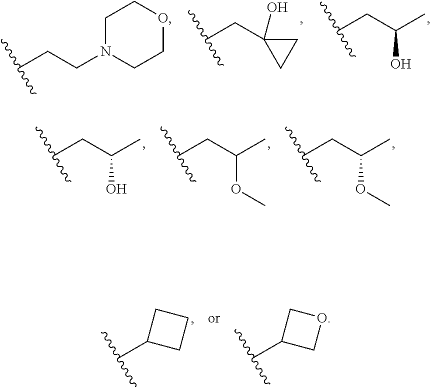

In some embodiments of the compounds of formula IA-2, Q¹ is C—H; Q² is C—R^{3A}; Q³ and Q⁴ are each C—R^{3B}; Q⁵ is N or C—R^{3B}; R^{3A} is H or —CH₃; and each R^{3B} is independently H or —CH₃.

In some embodiments of the compounds of formula IA-2, Q¹ is C—H; Q² is C—R^{3A}; Q³ and Q⁴ are each C—R^{3B}; Q⁵ is C—R^{3B}; R^{3A}—CH₃; and each R^{3B} is H.

In other embodiments of the compounds of formula IA-2, Q¹ is N; Q² is C—R^{3A}; Q³ and Q⁴ are each C—R^{3B}; Q⁵ is N or C—R^{3B}; R^{3A} is H or —CH₃; and each R^{3B} is independently H or —CH₃.

In some embodiments of the compounds of formula IA-2, each R^{7b} is independently H, —CH₃, —OCH₃, —OCH₂CH₃, or —CN; and R⁷⁰ is —CH₃, —CH₂CH₂OH,

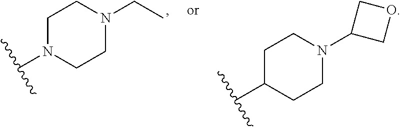

In other embodiments of the compounds of formula IA-2, each R^{7b} is independently H, or —CH₃; and R^{7c} is —CH₂CHF₂; —CH₂—CH(OH)—CH₃, —CH₂C(OH)(CH₃)₂, —CH₂CH₂CH₂OH, —C(CH₃)₂—CN, —CH₂CH₂OCH₃, —CH₂CH₂OCH₂CH₃, —CH₂CH₂OCH(CH₃)₂, —CH₂CH₂OCHF₂, —CH₂CH₂N(CH₃)₂,

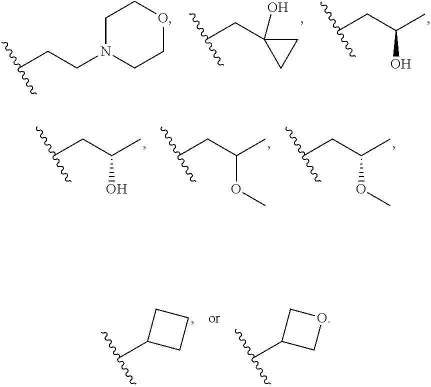

In some embodiments of the compounds of formula (IA-2), Q¹ is C—R^{3A}; Q² is C—R^{3A}; Q³ is C—R^{3B}; Q⁴ is C—R^{3B}; Q⁵ is C—R^{3B}; each R^{3A} is independently H or —CH₃; and each R^{3B} is independently H or —CH₃.

In some embodiments of the compounds of formula (IA-2), Q¹ is C—R^{3A}; Q² is C—R^{3A}; Q³ is C—R^{3B}; Q⁴ is C—R^{3B}; Q⁵ is C—R^{3B}; each R^{3B} is independently H or —CH₃; and the R^{3A} of Q¹ and the R^{3A} of Q², together with the carbon atoms to which they are attached, form a 5- or 6-membered cycloalkyl or heterocycloalkyl ring, or a phenyl ring.

In some embodiments, the compounds of formula IA are compounds of formula IA-3:

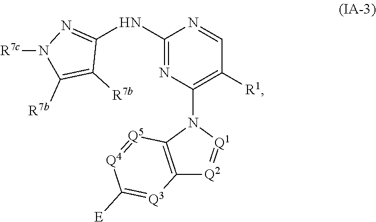

wherein each R^{7b} is independently H, —CH₃, —CH₂CH₃, isopropyl, —CH₂CF₃, —CHF₂, —CF₃, —CH₂-cyclopropyl, —CH₂CH₂OH,

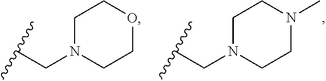

cyclopropyl, cyclobutyl,

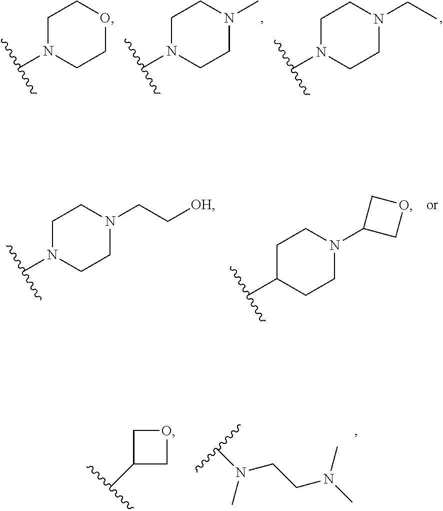

—OCH₃, —OCH₂CH₃,

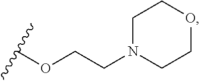 [SMILES: *OCCN1CCOCC1]

—CN or —F and R^{7c} is H, —CH₃, —CH₂CH₃, isopropyl, —CH₂CF₃, —CHF₂, —CF₃, —CH₂-cyclopropyl, —CH₂CH₂OH,

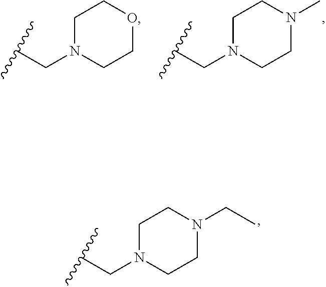

cyclopropyl, cyclobutyl,

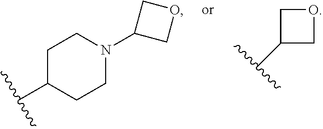 [SMILES: *C1CCN(C2COC2)CC1 ; *C1COC1]

In some embodiments of the compounds of formula IA-3, R^{7c} is —C(O)OC(CH₃)₃.

In some embodiments of the compounds of formula IA-3, Q¹ is C—H; Q² is C—R^{3A}; Q³ and Q⁴ are each C—R^{3B}; Q⁵ is N or C—R^{3B}; R^{3A} is H or —CH₃; and each R^{3B} is independently H or —CH₃.

In some embodiments of the compounds of formula IA-3, Q¹ is C—H; Q² is C—R^{3A}; Q³ and Q⁴ are each C—R^{3B}; Q⁵ is C—R^{3B}; R^{3A}—CH₃; and each R^{3B} is H.

In other embodiments of the compounds of formula IA-3, Q¹ is N; Q² is C—R^{3A}; Q³ and Q⁴ are each C—R^{3B}; Q⁵ is N or C—R^{3B}; R^{3A} is H or —CH₃; and each R^{3B} is independently H or —CH₃.

In some embodiments of the compounds of formula IA-3, each R^{7b} is independently H, or —CH₃; and R^{7c} is —CH₃, —CH₂CH₃, —CH₂CH₂OH, cyclopropyl, or

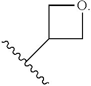 [SMILES: *C1COC1]

In some embodiments, the compounds of formula IA are compounds of formula IA-4:

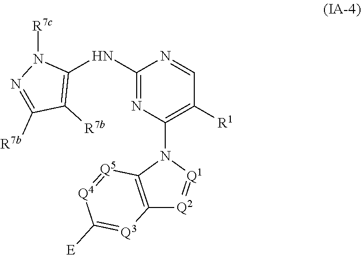

wherein each R^{7b} is independently H, —CH₃, —CH₂CH₃, isopropyl, —CH₂CF₃—CHF₂, —CF₃, —CH₂-cyclopropyl, —CH₂CH₂OH,

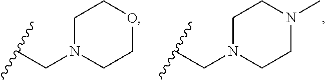

cyclopropyl, cyclobutyl,

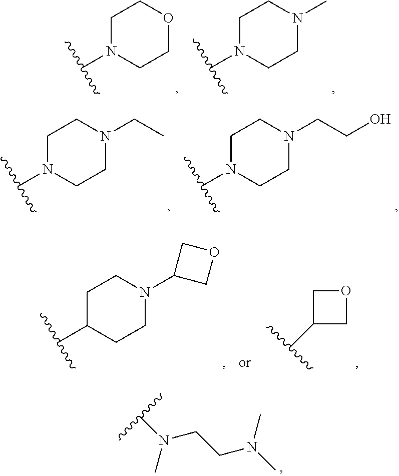

—OCH₃, —OCH₂CH₃,

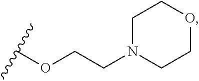 [SMILES: *OCCN1CCOCC1]

—CN, or —F; and R^{7c} is H—CH₃, —CH₂CH₃, isopropyl, —CH₂CF₃, —CHF₂, —CF₃, —CH₂-cyclopropyl, —CH₂CH₂OH,

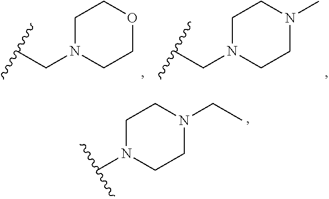

cyclopropyl, cyclobutyl,

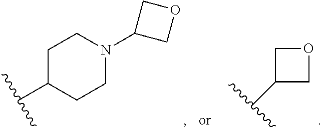 [SMILES: *C1CCN(C2COC2)CC1 ; *C1COC1]

In some embodiments of the compounds of formula IA-4, Q¹ is C—H; Q² is C—R^{3A}; Q³ and Q⁴ are each C—R^{3B}; Q⁵ is N or C—R^{3B}; R^{3A} is H or —CH₃; and each R^{3B} is independently H or —CH₃.

In some embodiments of the compounds of formula IA-4, Q¹ is C—H; Q² is C—R^{3A}; Q³ and Q⁴ are each C—R^{3B}; Q⁵ is C—R^{3B}; R^{3A}—CH₃; and each R^{3B} is H.

In other embodiments of the compounds of formula IA-4, Q¹ is N; Q² is C—R^{3A}; Q³ and Q⁴ are each C—R^{3B}; Q⁵ is N or C—R^{3B}; R^{3A} is H or —CH₃; and each R^{3B} is independently H or —CH₃.

In some embodiments of the compounds of formula IA-4, each R^{7b} is independently H or —CH₃, and R^{7c} is —CH₃.

In some embodiments, the compounds of formula IA are compounds of formula IA-5:

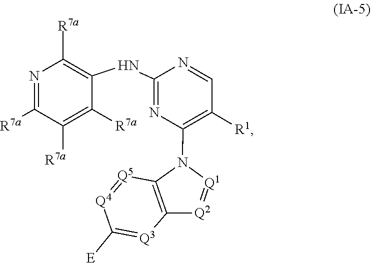

wherein each R^{7a} is independently H, —CH₃, —CH₂CH₃, isopropyl, —CH₂CF₃, —CHF₂, —CF₃, —CH₂-cyclopropyl, —CH₂CH₂OH,

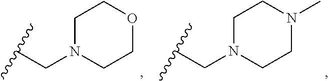

cyclopropyl, cyclobutyl,

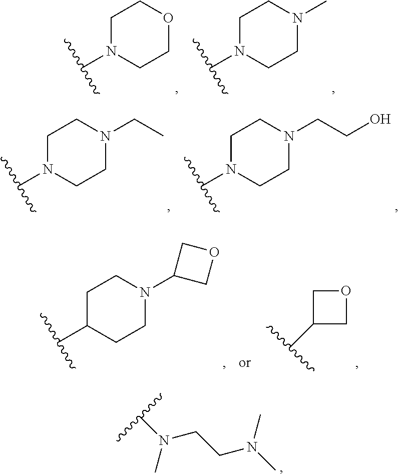

—OCH₃, —OCH₂CH₃,

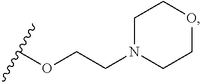 [SMILES: *OCCN1CCOCC1]

### —CN, or —F.

In some embodiments of the compounds of formula IA-5, Q¹ is C—H; Q² is C—R^{3A}; Q³ and Q⁴ are each C—R^{3B}; Q⁵ is N or C—R^{3B}; R^{3A} is H or —CH₃; and each R^{3B} is independently H or —CH₃.

In some embodiments of the compounds of formula IA-5, Q¹ is C—H; Q² is C—R^{3A}; Q³ and Q⁴ are each C—R^{3B}; Q⁵ is C—R^{3B}; R^{3A}—CH₃; and each R^{3B} is H.

In other embodiments of the compounds of formula IA-5, Q¹ is N; Q² is C—R^{3A}; Q³ and Q⁴ are each C—R^{3B}; Q⁵ is N or C—R^{3B}; R^{3A} is H or —CH₃; and each R^{3B} is independently H or —CH₃.

In some embodiments of the compounds of formula IA-5, three of the R^{7a} are H, and the other R^{7a} is

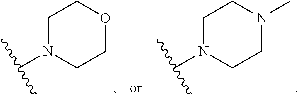

In other embodiments of the compounds of formula IA-5, three of the R^{7a} are H, and the other R^{7a} is —CH₃, or —F.

In some embodiments, the compounds of formula IA are compounds of formula IA-6:

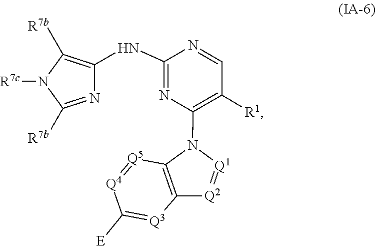

wherein each R^{7b} is independently H, —CH₃, —CH₂CH₃, isopropyl, —CH₂CF₃, —CHF₂, —CF₃, —CH₂-cyclopropyl, —CH₂CH₂OH,

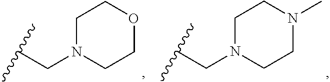

cyclopropyl, cyclobutyl,

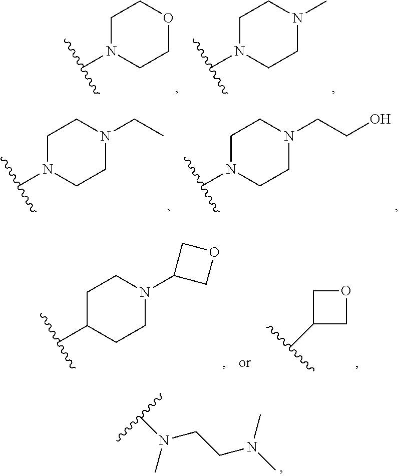

—OCH₃, —OCH₂CH₃,

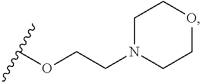 [SMILES: *OCCN1CCOCC1]

—CN, or —F; and R^{7c} is H, —CH₃, —CH₂CH₃, isopropyl, —CH₂CF₃, —CHF₂, —CF₃, —CH₂-cyclopropyl, —CH₂CH₂OH,

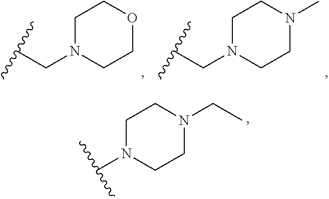

cyclopropyl, cyclobutyl,

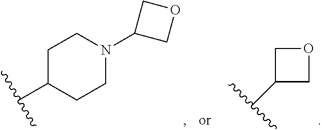 [SMILES: *C1CCN(C2COC2)CC1 ; *C1COC1]

In some embodiments of the compounds of formula IA-6, Q¹ is C—H; Q² is C—R^{3A}; Q³ and Q⁴ are each C—R^{3B}; Q⁵ is N or C—R^{3B}; R^{3A} is H or —CH₃; and each R^{3B} is independently H or —CH₃.

In some embodiments of the compounds of formula IA-6, Q¹ is C—H; Q² is C—R^{3A}; Q³ and Q⁴ are each C—R^{3B}; Q⁵ is C—R^{3B}; R^{3A}—CH₃; and each R^{3B} is H.

In other embodiments of the compounds of formula IA-6, Q¹ is N; Q² is C—R^{IA}; Q³ and Q⁴ are each C—R^{3B}; Q⁵ is N or C—R^{3B}; R^{3A} is H or —CH₃; and each R^{3B} is independently H or —CH₃.

In some embodiments of the compounds of formula IA-6, each R^{7b} is H, and R^{7c} is —CH₃.

In some embodiments of the disclosure, the compounds of formula I are compounds of formula IB:

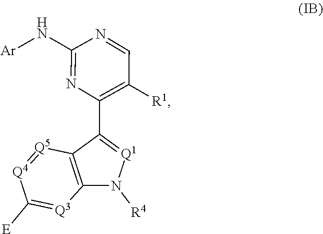

or pharmaceutically acceptable salts thereof, wherein Ar, R¹, Q¹, R⁴, Q³, Q⁴, Q⁵, and E have the definitions described above for formula I.

In some embodiments of the compounds of formula IB, Q¹ is C—H; R⁴ is —CH₃; Q³, Q⁴, and Q⁵ are each independently N or C—R^{3B}; and each R^{3B} is independently H or —CH₃.

In some embodiments of the compounds of formula IB, Q¹ is N; R⁴ is —CH₃, each Q³, Q⁴, and Q⁵ is N or C—R^{3B}; and each R^{3B} is independently H or —CH₃.

In some embodiments, the compounds of formula IB are compounds of formula IB-1:

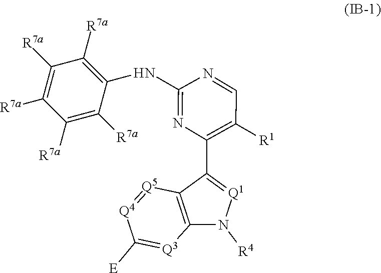

wherein each R^{7a} is independently H, —CH₃, —CH₂CH₃, isopropyl, —CH₂CF₃, —CHF₂, —CF₃, —CH₂-cyclopropyl, —CH₂CH₂OH,

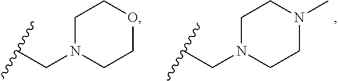

cyclopropyl, cyclobutyl,

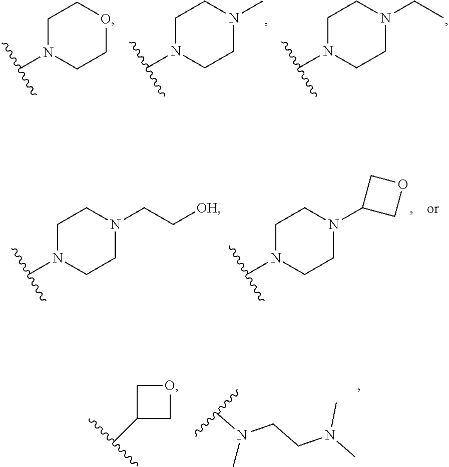

—OCH₃, —OCH₂CH₃,

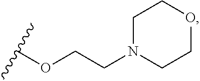 [SMILES: *OCCN1CCOCC1]

### —CN, or —F.

In some embodiments of the compounds of formula IB-1, Q¹ is C—H; R⁴ is —CH₃; Q³, Q⁴, and Q⁵ are each independently N or C—R^{3B}; and each R^{3B} is independently H or —CH₃.

In some embodiments of the compounds of formula IB-1, Q¹ is N; R⁴ is —CH₃, each Q³, Q⁴, and Q⁵ is N or C—R^{3B}; and each R^{3B} is independently H or —CH₃.

In some embodiments of the compounds of formula IB-1, each R^{7a} is independently H,

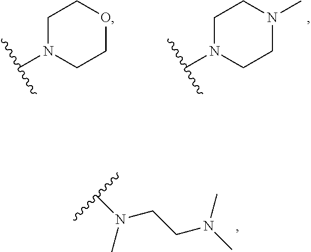

### or —OCH₃.

In some embodiments, the compounds of formula IB are compounds of formula IB-2:

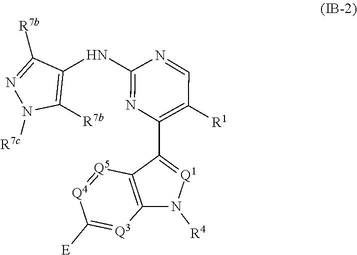

wherein each R^{7b} is independently H—CH₃—CH₂CH₃, isopropyl, —CH₂CF₃, —CHF₂, —CF₃, —CH₂-cyclopropyl, —CH₂CH₂OH,

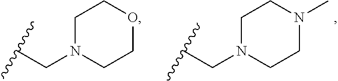

cyclopropyl, cyclobutyl,

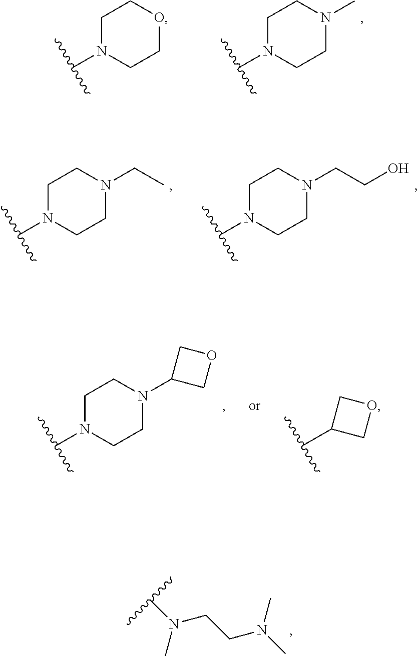

—OCH₃, —OCH₂CH₃,

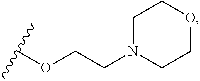 [SMILES: *OCCN1CCOCC1]

### —CN, or —F;

and R^{7c} is H, —CH₃—CH₂CH₃, isopropyl, —CH₂CF₃—CHF₂, —CF₃, —CH₂-cyclopropyl, —CH₂CH₂OH,

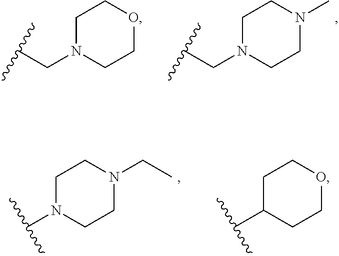

cyclopropyl, cyclobutyl,

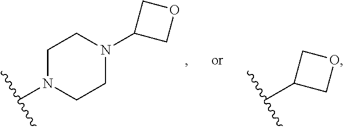 [SMILES: *N1CCN(C2COC2)CC1 ; *C1COC1]

In some embodiments of the compounds of formula IB-2, R^{7c} is —CH₂CHF₂.

In some embodiments of the compounds of formula IB-2, Q¹ is C—H; R⁴ is —CH₃; Q³, Q⁴, and Q⁵ are each independently N or C—R^{3B}; and each R^{3B} is independently H or —CH₃.

In some embodiments of the compounds of formula IB-2, Q¹ is N; R⁴ is —CH₃, each Q³, Q⁴, and Q⁵ is N or C—R^{3B}; and each R^{3B} is independently H or —CH₃.

In some embodiments of the compounds of formula IB-2, each R^{7b} is H and R^{7c} is —CH₃, —CH₂CH₃, isopropyl, —CH₂-cyclopropyl, or —CH₂CH₂OH.

In some embodiments, the compounds of formula IB are compounds of formula IB-3:

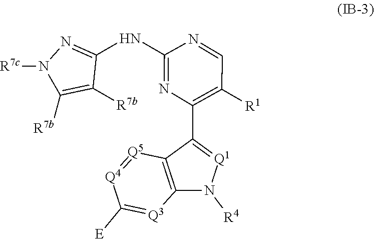

wherein each R^{7b} is independently H, —CH₃, —CH₂CH₃, isopropyl, —CH₂CF₃, —CHF₂, —CF₃, —CH₂-cyclopropyl, —CH₂CH₂OH,

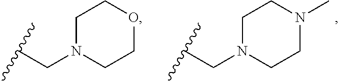

cyclopropyl, cyclobutyl,

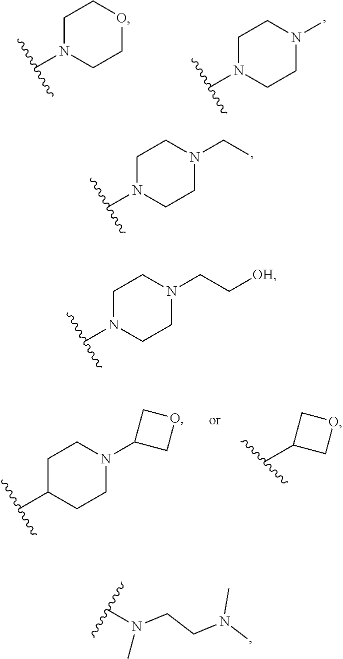

—OCH₃, —OCH₂CH₃,

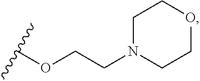 [SMILES: *OCCN1CCOCC1]

### —CN, or —F;

and R^{7c} is H, —CH₃, —CH₂CH₃, isopropyl, —CH₂CF₃—CHF₂—CF₃, —CH₂-cyclopropyl, —CH₂CH₂OH,

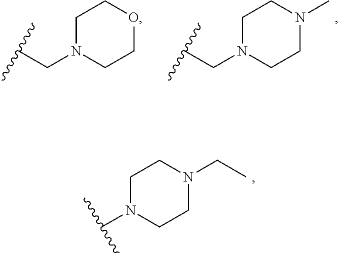

cyclopropyl, cyclobutyl,

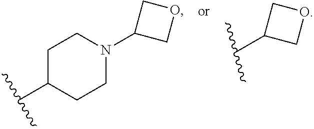 [SMILES: *C1CCN(C2COC2)CC1 ; *C1COC1]

In some embodiments of the compounds of formula IB-3, Q¹ is C—H; R⁴ is —CH₃; Q³, Q⁴, and Q⁵ are each independently N or C—R^{3B}; and each R^{3B} is independently H or —CH₃.

In some embodiments of the compounds of formula IB-3, Q¹ is N; R⁴ is —CH₃, each Q³, Q⁴, and Q⁵ is N or C—R^{3B}; and each R^{3B} is independently H or —CH₃.

In some embodiments of the compounds of formula IB-3, R^{7b} is independently H or —CH₃, and R^{7c} is H, —CH₃, —CH₂CH₃, CH₂CH₂OH, or cyclopropyl.

In some embodiments, the compounds of formula IB are compounds of formula IB-4:

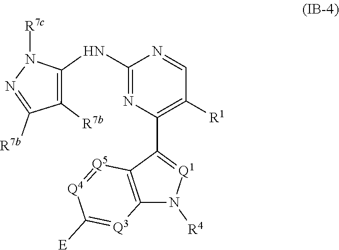

wherein each R^{7b} is independently H, —CH₃, —CH₂CH₃, isopropyl, —CH₂CF₃, —CHF₂, —CF₃, —CH₂-cyclopropyl, —CH₂CH₂OH,

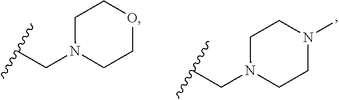

cyclopropyl, cyclobutyl,

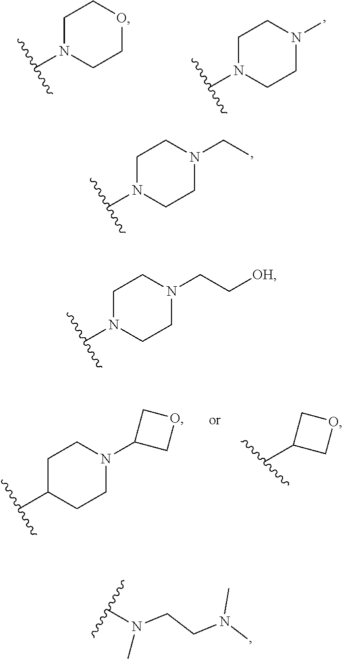

—OCH₃—OCH₂CH₃,

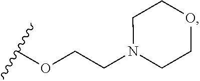 [SMILES: *OCCN1CCOCC1]

### —CN, or —F;

and R^{7c} is H—CH₃N—CH₂CH₃, isopropyl, CH₂CF₃, CHF₂, CF₃, —CH₃-cyclopropyl, —CH₂CH₂OH,

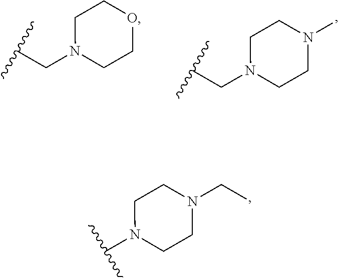

cyclopropyl, cyclobutyl,

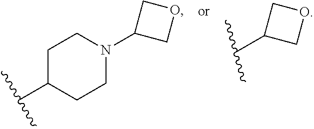 [SMILES: *C1CCN(C2COC2)CC1 ; *C1COC1]

In some embodiments of the compounds of formula IB-4, Q¹ is C—H; R⁴ is —CH₃; Q³, Q⁴, and Q⁵ are each independently N or C—R^{3B}; and each R^{3B} is independently H or —CH₃.

In some embodiments of the compounds of formula IB-4, Q¹ is N; R⁴ is —CH₃, each Q³, Q⁴, and Q⁵ is N or C—R^{3B}; and each R^{3B} is independently H or —CH₃.

In some embodiments of the compounds of formula IB-4, R^{7b} is independently H or —CH₃, and R^{7c} is —CH₃.

In some embodiments, the compounds of formula IB are compounds of formula IB-5:

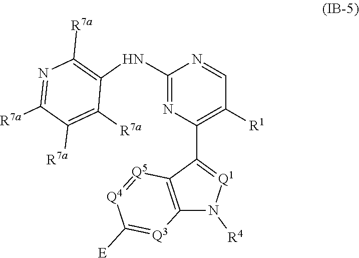

wherein each R^{7a} is independently H, —CH₃—CH₂CH, isopropyl, —CH₂CF₃, —CHF₂, —CF₃, —CH₂-cyclopropyl, —CH₂CH₂OH,

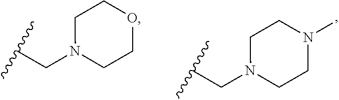

cyclopropyl, cyclobutyl,

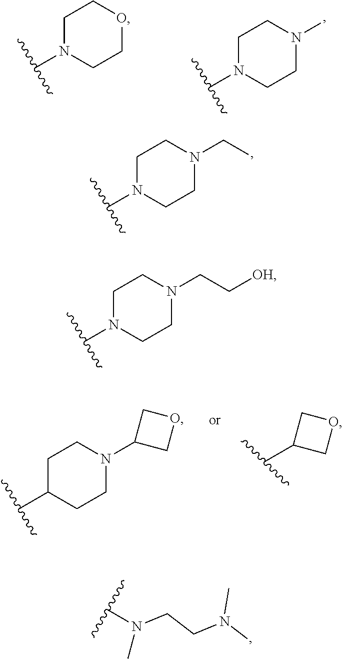

—OCH₃, —OCH₂CH₃,

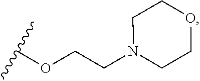 [SMILES: *OCCN1CCOCC1]

### —CN, or —F.

In some embodiments of the compounds of formula IB-5, Q¹ is C—H; R⁴ is —CH₃, Q³, Q⁴, and Q⁵ are each independently N or C—R^{3B}; and each R^{3B} is independently H or —CH₃.

In some embodiments of the compounds of formula IB-5, Q¹ is N; R⁴ is —CH₃, each Q³, Q⁴, and Q⁵ is N or C—R^{3B}; and each R^{3B} is independently H or —CH₃.

In some embodiments of the compounds of formula IB-5, three of the R^{7a} are H, and the other R^{7a} is

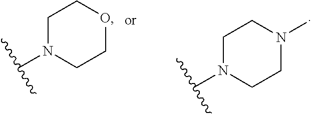

In some embodiments, the compounds of formula IB are compounds of formula IB-6:

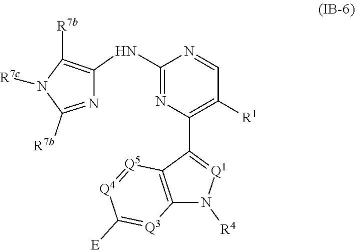

wherein each R^{7b} is independently H, —CH₃, —CH₂CH₃, isopropyl, —CH₂CF₃, —CHF₂, —CF₃, —CH₂-cyclopropyl, —CH₂CH₂OH,

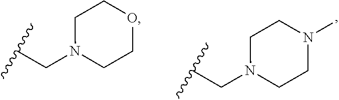

cyclopropyl, cyclobutyl,

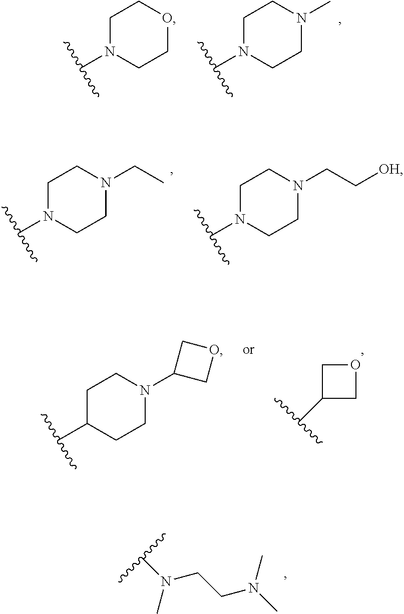

—OCH₃, —OCH₂CH₃,

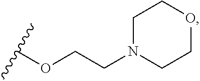 [SMILES: *OCCN1CCOCC1]

### —CN, or —F;

and R^{7c} is H, —CH₃, —CH₂CH₃, isopropyl, —CH₂CF₃, —CHF₂—CF₃, —CH₂-cyclopropyl, —CH₂CH₂OH,

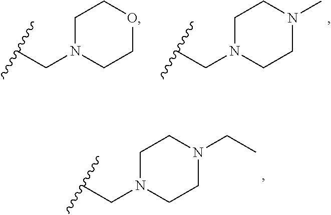

cyclopropyl, cyclobutyl,

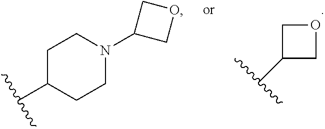 [SMILES: *C1CCN(C2COC2)CC1 ; *C1COC1]

In some embodiments of the compounds of formula IB-6, Q¹ is C—H; R⁴ is —CH₃; Q³, Q⁴, and Q⁵ are each independently N or C—R^{3B}; and each R^{3B} is independently H or —CH₃.

In some embodiments of the compounds of formula IB-6, Q¹ is N; R⁴ is —CH₃, each Q³, Q⁴, and Q⁵ is N or C—R^{3B}; and each R^{3B} is independently H or —CH₃.

In some embodiments of the compounds of formula IB-6, each R^{7b} is H, and R^{7′} is —CH₃.

In some embodiments, the compounds of formula I include the following:

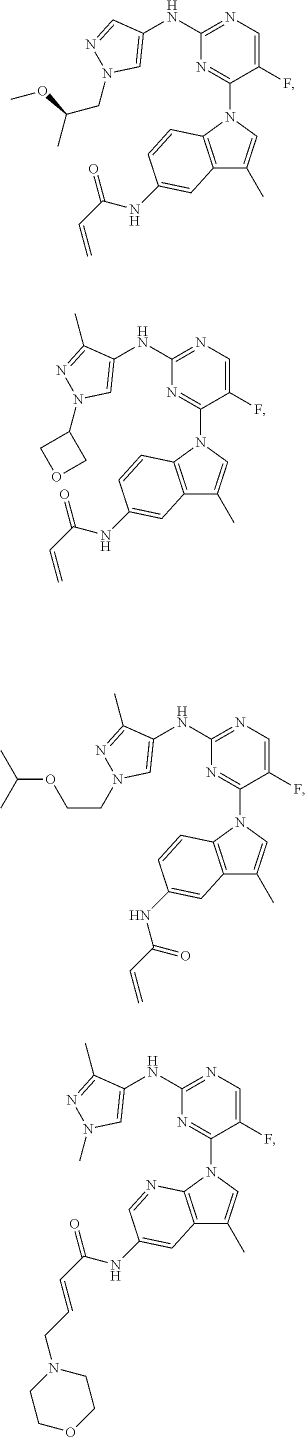 [SMILES: C=CC(=O)Nc1ccc2c(c1)c(C)cn2-c1nc(Nc2cnn(C[C@@H](C)OC)c2)ncc1F ; C=CC(=O)Nc1ccc2c(c1)c(C)cn2-c1nc(Nc2cn(C3COC3)nc2C)ncc1F ; C=CC(=O)Nc1ccc2c(c1)c(C)cn2-c1nc(Nc2cn(CCOC(C)C)nc2C)ncc1F ; Cc1nn(C)cc1Nc1ncc(F)c(-n2cc(C)c3cc(NC(=O)/C=C/CN4CCOCC4)cnc32)n1]

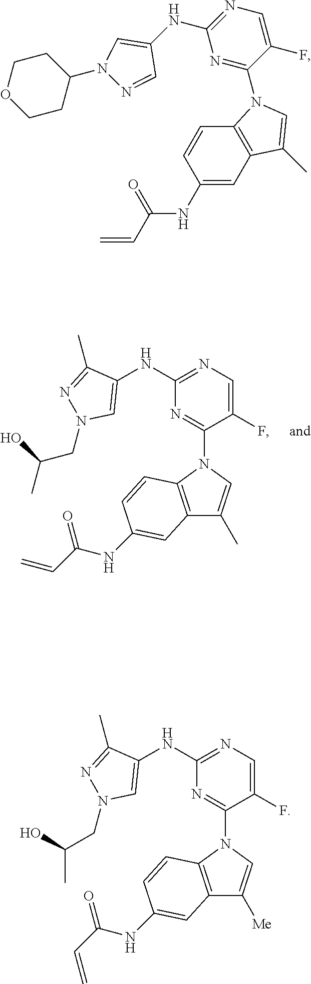 [SMILES: C=CC(=O)Nc1ccc2c(c1)c(C)cn2-c1nc(Nc2cnn(C3CCOCC3)c2)ncc1F ; C=CC(=O)Nc1ccc2c(c1)c(C)cn2-c1nc(Nc2cn(C[C@@H](C)O)nc2C)ncc1F ; C=CC(=O)Nc1ccc2c(c1)c(C)cn2-c1nc(Nc2cn(C[C@@H](C)O)nc2C)ncc1F]

In some aspects, the disclosure is directed to compounds of formula (II),

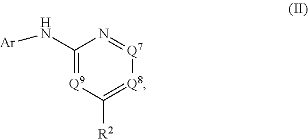

or pharmaceutically acceptable salts thereof,
wherein:
   Ar is a 6-10-membered aryl group or a 5-10-membered heteroaryl group, each optionally substituted;
   Q⁷ is N or CH;
   Q⁸ is N or C—R¹;
   Q⁹ is N or CH;
      wherein when Q⁹ is N, at least one of Q⁷ and Q⁸ is also N;
   R¹ is H, F, Cl, Br, C₁₋₆alkyl, CN, C₁₋₆haloalkyl, —C(O)NH₂, —C(O)NH(C₁-6alkyl), or —C(O)N(C₁₋₆alkyl)₂;
   R² is a 9-membered bicyclic heteroaryl comprising a 6-5-fused ring system wherein the 5-membered ring contains at least one nitrogen atom and the 6 membered ring is substituted with at least one E, wherein E is an electrophilic moiety that is capable of reacting with a nucleophile so as to form a covalent bond between an atom of the nucleophile and an atom of the electrophilic moiety.

In some embodiments of the compounds of formula (II), Q⁷ is N.

In some embodiments of the compounds of formula (II), Q⁷ is CH.

In some embodiments of the compounds of formula (II), Q⁸ is N.

In some embodiments of the compounds of formula (II), Q⁸ is C—R¹.

In some embodiments of the compounds of formula (II), Q⁹ is N.

In some embodiments of the compounds of formula (II), Q⁹ is CH.

In some embodiments of the disclosure, the compounds of formula II are compounds of formula IIA:

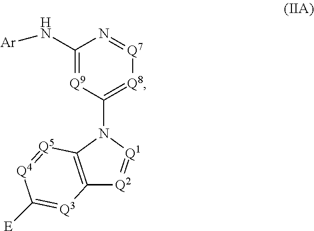

or pharmaceutically acceptable salts thereof, wherein Ar, R¹, Q¹; Q², Q³, Q⁴, Q⁵, and E have the definitions described above for formula I.

In some embodiments, the compounds of formula IIA are compounds of formula IIA-2:

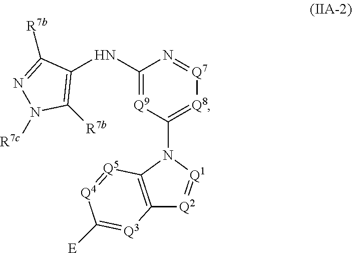

wherein R^{7b}, R^{7c}, R¹, Q¹, Q², Q³, Q⁴, Q⁵, and E have the definitions described above for formula I.

In some embodiments of the compounds of formula IIA-2, each R^{7b} is independently H or —CH₃; R^{7c} is H or —CH₃; Q¹, Q³, Q⁴, and Q⁵ are each CH; Q² is C—CH₃, and E is —NHC(O)CH═CH₂.

In some embodiments of the disclosure, the compounds of formula II are compounds of formula IB:

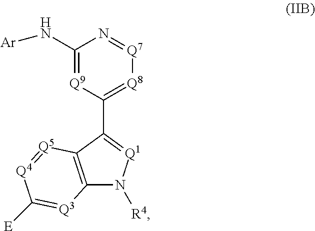

or pharmaceutically acceptable salts thereof, wherein Ar, R¹, Q¹, R⁴, Q³, Q⁴, Q⁵, and E have the definitions described above for formula I.

In some embodiments, the compounds of formula (II) include the following:

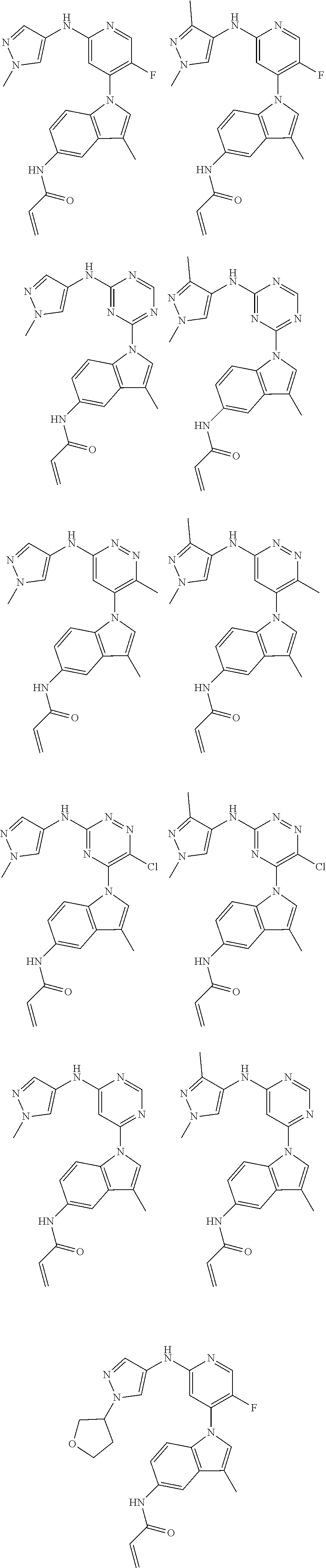

References herein to formula (I) or formula (II) or subgenera thereof are meant to encompass the identified formula and any subgenera of those formula disclosed herein.

Stereoisomers of compounds of formula (I) or formula (II) are also contemplated by the present disclosure. Thus, the disclosure encompasses all stereoisomers and constitutional isomers of any compound disclosed or claimed herein, including all enantiomers and diastereomers, or mixtures thereof.

Pharmaceutically acceptable salts and solvates of the compounds of formula (I) or formula (II) are also within the scope of the disclosure.

Isotopic variants of the compounds of formula (I) or formula (II) are also contemplated by the present disclosure.

It is to be appreciated that certain features of the invention which are, for clarity, described herein in the context of separate embodiments, may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. While an embodiment may be described as part of a series of steps or part of a more general structure, each said step may also be considered an independent embodiment in itself, combinable with others.

In some embodiments, the compounds of the disclosure include the compounds exemplified in the Examples below.

The FGFR receptors (FGFRl, FGFR2, FGFR3, and FGFR4) share several structural features in common, including three extracellular immunoglobulin-like (Ig) domains, a hydrophobic transmembrane domain, and an intracellular tyrosine kinase domain split by a kinase insert domain, followed by a cytoplasmic c-terminal tail (Johnson et al., Adv. Cancer Res. 60:1-40, 1993; and Wilkie et al., Curr. Biol. 5:500-507, 1995). In FGFR1, the kinase insert domain spans positions 582 to 595 of the alpha Al isoform of FGFR. In FGFR2, the kinase insert domain spans positions 585 to 598 of the FGFR2 Ille isoform. In FGFR3, the kinase insert domain spans positions 576 to 589 of the FGFR3 Ille isoform. In FGFR4, the kinase insert domain spans positions 571 to 584 of FGFR4 isoform 1. The c-terminal tail of FGFRs begins following the end of the tyrosine kinase domain and extends to the c-terminus of the protein. Several isoforms of each FGFR have been identified and are the result of alternative splicing of their mRNAs (Johnson et al., Mol. Cell. Biol. 11:4627-4634, 1995; and Chellaiah et al., J. Biol. Chem. 269:11620-11627, 1994).

A few of the receptor variants that result from this alternative splicing have different ligand binding specificities and affinities (Zimmer et al., J. Biol. Chem. 268:7899-7903, 1993; Cheon et al., Proc. Natl. Acad. Sci. U.S.A. 91:989-993, 1994; and Miki et al., Proc. Natl. Acad. Sci. U.S.A. 89:246-250, 1992). Protein sequences for FGFR proteins and nucleic acids encoding FGFR proteins are known in the art. Signaling by FGFRs regulates key biological processes including cell proliferation, survival, migration, and differentiation. Dysregulation of a FGFR gene, a FGFR protein, or expression or activity, or level of the same, has been associated with many types of cancer. For example, dysregulation of FGFRs can occur by multiple mechanisms, such as FGFR gene overexpression, FGFR gene amplification, activating mutations (e.g., point mutations or truncations), and chromosomal rearrangements that lead to FGFR fusion proteins. Dysregulation of a FGFR gene, a FGFR protein, or expression or activity, or level of the same, can result in (or cause in part) the development of a variety of different FGFR-associated cancers.

FGFR fusion proteins are known in the art. See, e.g., Baroy et al., PloS One; 11(9):e0163859. doi: 10.1371/journal.pone.0163859, 2016; Ren et al., Int. J Cancer, 139(4):836-40, 2016; Marchwicka et al., Cell Biosci., 6:7. doi: 10.1186/s13578-016-0075-9, 2016; PCT Patent Application Publication No. WO 2014/071419A2; U.S. Patent Application Publication No. 2015/0366866A1; PCT Patent Application Publication No. WO 2016/084883A1; PCT Patent Application Publication No. WO 2016/030509A1; PCT Patent Application Publication No. WO 2015/150900A2; PCT Patent Application Publication No. WO 2015/120094A2; Kasaian et al., BMC Cancer., 15:984, 2015; Vakil et al., Neuro-Oncology, 18:Supp. Supplement 3, pp. iii93. Abstract Number: LG-64, 17^{th} International Symposium on Pediatric Neuro-Oncology, Liverpool, United Kingdom, 2016; Astsaturov et al., Journal of Clinical Oncology, 34:Supp. Supplement 15, Abstract Number: 11504, 2016 Annual Meeting of the American Society of Clinical Oncology, Chicago, IL; Heinrich et al., Journal of Clinical Oncology, 34:Supp. Supplement 15, Abstract Number: 11012, 2016 Annual Meeting of the American Society of Clinical Oncology, Chicago, IL; Hall et al., Molecular Cancer Therapeutics, Vol. 14, No. 12, Supp.2, Abstract Number: B151, AACR-NCI-EORTC International Conference: Molecular Targets and Cancer Therapeutics, 2015; Reuther et al., Journal of Molecular Diagnostics, Vol. 17, No. 6, pp. 813, Abstract Number: ST02, 2015 Annual Meeting of the Association for Molecular Pathology, Austin, TX; Moeini et al., Clin. Cancer. Res., 22(2):291-300, 2016; Schrock et al, J Thorac. Oneal. pii S1556-0864(18)30674-9, 2018. doi: 10.1016/j.jtho.2018.05.027; Pekmezci et al, Acta Nurotapho/. Commun. 6(1):47. doi: 10.1186/s40478-018-0551-z; Lowery et al. Clin Cancer Res. pii: clincanres.0078.2018. doi: 10.1158/1078-0432.CCR-18-0078; Ryland et al. J Clin Patho/ pii: jclinpath-2018-205195, 2018. doi: 10.1136/jclinpath-2018-205195; Ferguson et al. J Neuropatho/Exp Neural 77(6):437-442, 2018. doi: 10.1093/jnen/nly022; Wu et al, BMC Cancer 18(1):343, 2018. doi: 10.1186/s12885-018-4236-6; Shibata et al, Cancer Sci 109(5):1282-1291, 2018. doi: 10.1111/cas.13582; Papdopoulos et al, Br J Cancer, 1117(11):1592-1599, 2017. doi: 10.1038/bjc.2017.330; Hall et al, PLoS One, 11(9):e1062594, 2016. doi: 10.1371/journal.pone.0162594; Johnson et al, Oncologist, 22(12):1478-1490, 2017. doi: 10.1634/theoncologist.2017-0242; Yang et al, Am J Hum Genet, 98(5):843-856, 2016. doi: 10.1016/j.ajhg.2016.03.017; U.S. Patent Application Publication No. 2013/009621; Babina and Turner, Nat Rev Cancer 17(5):318-332, 2017. doi: 10.1038/nrc.2017.8; Ryland et al, J Clin Patho., 2018 May 14. pii: jclinpath-2018-205195. doi: 10.1136/jclinpath-2018-205195; Kumar et al, Am J Clin Patho. 143(5):738-748, 2015. doi: 10.1309/AJCPUD6W1JLQQMNA; Grand et al, Genes Chromosomes Cancer 40(1):78-83, 2004. doi: 10.1002/gcc.20023; Reeser, et al, JMo/Diagn, 19(5):682-696, 2017. doi: 10.1016/j.jmoldx.2017.05.006; Basturk, et al, ModPatho/, 30(12):1760-1772, 2017. doi: 10.1038/modpathol.2017.60; Wang, et al, Cancer 123(20):3916-3924, 2017. doi: 10.1002/cncr.30837; Kim, et al, Oncotarget, 8(9):15014-15022, 2017. doi: 10.18632/oncotarget.14788; Busse, et al, Genes Chromosomes Cancer, 56(10):730-749, 2017. doi: 10.1002/gcc.22477; Shi, et al, J Transl Med., 14(1):339, 2016. doi: 10.1186/s12967-016-1075-6, each of which is incorporated by reference herein.

FGFR point mutations are known in the art. See, e.g., UniParc entry UPI00000534B8; UniParc entry UPI000000lCOF; UniParc entry UPI000002A99A; UniParc entry UPI000012A72A; UniParc entry UPI000059D1C₂; UniParc entry UPI000002A9AC; Uniparc entry UPI000012A72C; Uniparc entry UPI000012A72D; Uniparc entry UPI000013EOB8; Uniparc entry UPI0001CE06A3; Gen bank entry BAD92868.l; Ang et al., Diagn. Mo. Patho/. Feb. 24, 2014; U.S. Patent Application Publication No. 2011/0008347; Gallo et al., Cytokine Growth Factor Rev. 26:425-449, 2015; Davies et al., J Cancer Res. 65:7591, 2005; Kelleher et al., Carcinogenesis 34:2198, 2013; Cazier et al., Nat. Commun. 5:3756, 2014; Liu et al., Genet. Mo. Res. 13:1109, 2014; Trudel et al., Blood 107:4039, 2006; Gallo et al., Cytokine Growth Factor Rev. 26:425, 2015; Liao et al., Cancer Res. 73:5195-5205, 2013; Martincorena et al., Science 348:880 (2015); U.S. Patent Application Publication No. US2016/0235744A1; U.S. Pat. No. 9,254,288B2; U.S. Pat. No. 9,267,176B2; U.S. Patent Application Publication No. 52016/0215350A1; European Patent Application Publication No. EP3023101A1; PCT Patent Application Publication No. WO2016105503A1; Rivera et al., Acta. Neuropatho/.,131(6):847-63, 2016; Lo Iacono et al., Oncotarget., 7(12):14394-404, 2016; Deeken et al., Journal of Clinical Oncology, 34:Supp. Supplement 15, pp. iii93. Abstract Number: el 7520, 2016 Annual Meeting of the American Society of Clinical Oncology, Chicago, IL; Sullivan et al., Journal of Clinical Oncology, 34:Supp. Supplement 15, pp. iii93. Abstract Number: 11596, 2016 Annual Meeting of the American Society of Clinical Oncology, Chicago, IL; Nguyen et al., Molecular Cancer Therapeutics, Vol. 14, No. 12, Supp.2, Abstract Number: C₁₉₉, AACR-NCI-EORTC International Conference: Molecular Targets and Cancer Therapeutics, 2015; Li et al., Hum. Patho., 55:143-50, 2016; European Patent No. EP2203449B1; Yoza et al., Genes Cells., (10):1049-1058, 2016; U.S. Pat. No. 9,254,288B2; European Patent Application Publication No. 3023101A1; PCT Application Publication No. WO 2015/099127A1; European Patent No. EP2203449B1; Yoza et al., Genes Cells., (10):1049-1058, 2016; Bunney et al., EbioMedicine, 2(3):194-204, 2015; Byron et al., Neop/asia, 15(8):975-88, 2013; European Patent Application Publication No. EP3023101A1; PCT Application Publication No. WO 2015/099127A1; Thussbas et al., J. Clin. Oneal., 24(23):3747-55, 2006; Chell et al., Oncogene, 32(25):3059-70, 2013; Tanizaki et al, Cancer Res. 75(15):3149-3146 doi: 10.1158/0008-5472.CAN-14-3771; Yang et al, EBioMedicine pii 52352-3964(18)₃₀₂₁₈-4. doi: 10.1016/j.ebiom.2018.06.011; Jakobsen, et al Oncotarget 9(40):26195-26208, 2018. doi: 10.18632/oncotarget.25490; Stone, et al Acta Neuropatho/ 135(1):115-129, 2017. doi: 10.1007/s00401-017-1773-z; Pekmezci et al, Acta Nurotaphol. Commun. 6(1):47. doi: 10.1186/s40478-018-0551-z; De Mattos-Arruda et al, Oncotarget 9(29):20617-20630, 2018. doi:10.18632/oncotarget.25041; Oliveira et al, JExp C/in Cancer Res 37(1):84, 2018. doi: 10.1186/sl3046-018-0746-y; Cha et al, Mo/Oneal 12(7):993-1003, 2018. doi: 10.1002/1878-0261.12194; Ikeda et al, Oncologist, 23(5):586-593, 2018. doi: 10.1634/theoncologist.2017-0479; Pelaez-Garda et al, PLoS One, 8(5):e63695, 2013. doi: 10.1371/journal.pone.0063695; Shimada et al, Oncotarget, 8(55):93567-93579, 2017. doi: 10.18632/oncotarget.20510; Welander et al, World JSurg, 42(2):482-489, 2018. doi: 10.1007/s00268-0l 7-4320-0; Chandrani et al, Ann Oneal, 28(3):597-603, 2017. doi: 10.1093/annonc/mdw636; Dalin et al, Nat Commun, 8(1):1197, 2017. doi: 10.1038/s41467-017-01178-z; Taurin et al, Intl Gyneco/Cancer, 28(1):152-160, 2018. doi: 10.1097/IGC.0000000000001129; Haugh et al, J Invest Dermatol 138(2):384-393, 2018. doi: 10.1016/j.jid.2017.08.022; Babina and Turner, Nat Rev Cancer 17(5):318-332, 2017. doi: 10.1038/nrc.2017.8; Greenman et al, Nature 446(7132):153-158, 2007. doi: 10.1038/nature05610; Helsten et al, Clin Cancer Res, 22(1):259-267, 2016. doi: 10.1158/1078-0432.CCR-14-3212; Kim et al, BMC Urol, 18:68, 2018. doi: 10.1186/s12894-018-0380-1; Goyal et al, Cancer Discov, 7(3):252-263, 2017. doi: 10.1158/2159-8290.CD-16-1000; Premov et al, Oncogene, 36(22):3168-3177, 2017. doi: 10.1038/onc.2016.464; Geelvink et al, IntJMo/Sci. 19(9): pii:E2548, 2018. doi: 10.3390/ijms19092548; Lee et al, Exp TherMed. 16(2):1343-1349, 2018. doi: 10.3892/etm.2018.6323; Kas et al, Cancer Res, 78(19):5668-5679, 2018. doi: 10.1158/0008-5472.CAN-18-0757; Chesi et al, Blood, 97(3):729-736, 2001. PMID: 11157491. Ge, et al, Am J Cancer Res. 7(7):1540-1553, 2017. PMID: 28744403; Jiao et al, Nat Genet, 45(12):1470-1473, 2013. doi: 10.1038/ng.2813; Jusakul et al, Cancer Discov. 7(10):1116-1135, 2017. doi: 10.1158/2159-8290.CD-17-0368; Guyard et al, Respir Res., 18(1):120, 2018. doi: 10.1186/s12931-017-0605-y; Paik et al, Clin Cancer Res., 23(18):5366-5373, 2017. doi: 10.1158/1078-0432.CCR-17-0645; Roy et al, ModPatho/., 30(8):1133-1143, 2017. doi: 10.1038/modpathol.2017.33; Chakrabarty et al, Br J Cancer, 117(1):136-143, 2017. doi: 10.1038/bjc.2017.148; Hoang et al, Sci Transl Med., 5(197):197ra102. doi: 10.1126/scitranslmed.3006200; Kim et al, Ann Oneal., 28(6):1250-1259. doi: 10.1093/annonc/mdx098, each of which is incorporated by reference herein.

Compounds of the disclosure have been found to inhibit FGFRl, FGFR2 and/or FGFR3, and are therefore believed to be useful for treating diseases and disorders which can be treated with an inhibitor of FGFRl, FGFR2, FGFR3 and/or FGFR4. For example, compounds of the disclosure can be useful in treating FGFR-associated diseases and disorders, e.g., proliferative disorders such as cancers, including hematological cancers and solid tumor, and angiogenesis-related disorders. Compounds of the disclosure may also be useful in treating disorders arising from autosomal dominant mutations in FGFR, e.g., FGFR3, including, for example, developmental disorders. Developmental disorders to be treated with compounds of the disclosure include Achondroplasia (Ach) and related chondrodysplasia syndromes, including Hypochondroplasia (Hch), Severe Achondroplasia with Developmental Delay and Acanthosis Nigricans (SADDAN), and Thanatophoric dysplasia (TD).

Non-limiting examples of FGFR-associated diseases and disorders include Acanthosis nigricans, Achondroplasia, Apert syndrome, Beare-Stevenson syndrome (BSS), Camptodactyly, tall stature, and hearing loss syndrome (CATSHL) syndrome, cleft lip and palate, congenital heart disease (e.g., associated with ambiguous genitalia), craniosynostosis, Crouzon syndrome, ectrodactyly, encephalocraniocutaneous lipomatosis, Hartsfield syndrome, hypochondroplasia, hypogonadoropic hypogonadism (e.g., hypogonadotropic hypogonadism 2 with or without anosmia, Kallman syndrome), ichthyosis vulgaris and/or atopic dermatitis, Jackson-Weiss syndrome, lethal pulmonary acinar dysplasia, microphthalmia, Muenke coronal craniosynostosis, osteoglophonic dysplasia, Pfeiffer syndrome, seborrheic keratosis, syndactyly, thanatophoric dysplasia (e.g., type I or type II), trigonocephaly 1 (also called metopic craniosynostosis), and tumor-induced osteomalacia. Non-limiting examples of FGFR1 associated diseases and disorders include congenital heart disease (e.g., associated with ambiguous genitalia), craniosynostosis, encephalocraniocutaneous lipomatosis, Hartsfield syndrome, hypogonadoropic hypogonadism (e.g., hypogonadotropic hypogonadism 2 with or without anosmia, Kallman syndrome), ichthyosis vulgaris and/or atopic dermatitis, Jackson-Weiss syndrome, osteoglophonic dysplasia, Pfeiffer syndrome, trigonocephaly 1 (also called metopic craniosynostosis), and tumor-induced osteomalacia.

Non-limiting examples of FGFR2-associated diseases and disorders include Apert syndrome, Beare-Stevenson syndrome (BSS), Crouzon syndrome, ectrodactyly, Jackson-Weiss syndrome, lethal pulmonary acinar dysplasia, Pfeiffer syndrome, and syndactyly. Non-limiting examples of FGFR3-associated diseases and disorders include acanthosis nigricans, achondroplasia, Camptodactyly, tall stature, and hearing loss syndrome (CATSHL) syndrome, cleft lip and palate, craniosynostosis, hypochondroplasia, microphthalmia, Muenke coronal craniosynostosis, seborrheic keratosis, and thanatophoric dysplasia (e.g., type I or type II).

See also, See UniParc entry UP100000534B8; UniParc entry UPI0000001COF; Uni Pare entry UPI000002A99A; UniParc entry UPI000012A72A; Yong-Xing et al., Hum. Mol. Genet. 9(13):2001-2008, 2000; Eeva-Maria Laitinen et al., PLoS One 7(6):e39450, 2012; Hart et al., Oncogene 19(29):3309-3320, 2000; Shiang et al., Cell 76:335-342, 1994; Rosseau et al., Nature 371:252-254, 1994; Tavormina et al., Nature Genet. 9:321-328, 1995; Bellus et al., Nature Genet. 10:357-359, 1995; Muenke et al., Nature Genet. 8:269-274, 1994; Rutland et al., Nature Genet. 9:173-176, 1995; Reardon et al., Nature Genet. 8:98-103, 1994; Wilkie et al., Nature Genet. 9:165-172, 1995; Jabs et al., Nature Genet. 8:275-279, 1994; Japanese Patent No. JP05868992B2; Ye et al., Plast. Reconstr. Surg., 137(3):952-61, 2016; U.S. Pat. No. 9,447,098B2; Bellus et al., Am. J. Med. Genet. 85(1):53-65, 1999; PCT Patent Application Publication No. WO2016139227A1; Australian Patent Application Publication No. AU2014362227A1; Chinese Patent No. CN102741256B; Ohishi et al., Am. J. Med. Genet. A., doi: 10.1002/ajmg.a.37992, 2016; Nagahara et al., Clin. Pediatr. Endocrinol., 25(3): 103-106, 2016; Hibberd et al., Am. J. Med. Genet. A., doi: 10.1002/ajmg.a.37862, 2016; Dias et al., Exp. Mol. Pathol., 101(1):116-23, 2016; Lin et al., Mol. Med. Rep., 14(3):1941-6, 2016; Barnett et al., Hum. Mutat., 37(9):955-63, 2016; Krstevska-Konstantinova et al., Med. Arch., 70(2):148-50, 2016; Kuentz et al., Br. J. Dermatol., doi: 10.1111/bjd.14681, 2016; Ron et al., Am. J. Case Rep., 15; 17:254-8, 2016; Fernandes et al., Am. J. Med. Genet. A., 170(6):1532-7, 2016; Lindy et al., Am. J. Med. Genet. A., 170(6):1573-9, 2016; Bennett et al., Am. J. Hum. Genet., 98(3):579-87, 2016; lchiyama et al., J. Eur. Acad. Dermatol. Venereal., 30(3):442-5, 2016; Zhao et al., Int. J. Clin. Exp. Med., 8(10):19241-9, 2015; Hasegawa et al., Am. J. Med. Genet. A., 170A(5):1370-2, 2016; Legeai-Mallet, Endocr. Dev., 30:98-105, 2016; Takagi, Am. J. Med. Genet. A., 167A(ll):2851-4, 2015; Goncalves, Fertil. Steril., 104(5):1261-7.el, 2015; Miller et al., Journal of Clinical Oncology, 34:Supp. Supplement 15, pp. iii93. AbstractNumber: e22500, 2016 Annual Meeting of the American Society of Clinical Oncology, Chicago, IL; Sarabipour et al., J. Mol. Biol., 428(20):3903-3910, 2016; Escobar et al., Am. J. Med. Genet. A., 170(7):1908-11, 2016; Mazen et al., Sex Dev., 10(1):16-22, 2016; Taylan et al., J Allergy Clin Immunol, 136(2):507-9, 2015. doi: 10.1016/j.jaci.2015.02.010; Kant et al, EuroJourn Endocrinol, 172(6):763-770, 2015. doi: 10.1530/EJE-14-0945; Gonzalez-Del Angel et al, Am J med Genet A, 176(1):161-166, 2018. doi: 10.1002/ajmg.a.38526; Lei and Deng, Int J Biol Sci 13(9):1163:1171, 2017. doi: 10.7150/ijbs.20792; Lajeunie et al, Eur J Hum Genet, 14(3):289-298, 2006. doi: 10.1038/sj.ejhg.5201558; Karadimas et al, Prenat Diagn, 26(3):258-261, 2006. doi: 10.1002/pd.1392; lbrahimi et al, Hum Mo Genet 13(19):2313-2324, 2004. doi: 10.1093/hmg/ddh235; Trarbach et al, J Cin Endocrinol Metab., 91(10):4006-4012, 2006. doi: 10.1210/jc.2005-2793; Dode et al, Nat Genet, 33(4):463-465, 2003. doi: 10.1038/ng1122, each of which is incorporated by reference herein.

The term “angiogenesis-related disorder” means a disease characterized in part by an increased number or size of blood vessels in a tissue in a subject or patient, as compared to a similar tissue from a subject not having the disease. Non-limiting examples of angiogenesis-related disorders include: cancer (e.g., any of the exemplary cancers described herein, such as prostate cancer, lung cancer, breast cancer, bladder cancer, renal cancer, colon cancer, gastric cancer, pancreatic cancer, ovarian cancer, melanoma, hepatoma, sarcoma, and lymphoma), exudative macular degeneration, proliferative diabetic retinopathy, ischemic retinopathy, retinopathy of prematurity, neovascular glaucoma, iritis rubeosis, corneal neovascularization, cyclitis, sickle cell retinopathy, and pterygium.

Compounds of the disclosure inhibit wild-type FGFR1, FGFR2, FGFR3, and/or FGFR4. In other aspects, compounds of the disclosure inhibit a mutated FGFR1, FGFR2, FGFR3, and/or FGFR4. In other aspects, compounds of the disclosure inhibit FGFR1, FGFR2, FGFR3, and/or FGFR4 that includes an FGFR kinase inhibitor mutation.

In some embodiments of any of the methods or uses described herein, the cancer (e.g., FGFR-associated cancer) is a hematological cancer. In some embodiments of any of the methods or uses described herein, the cancer (e.g., FGFR-associated cancer) is a solid tumor. In some embodiments of any of the methods or uses described herein, the cancer (e.g., FGFR-associated cancer) is a lung cancer (e.g., small cell lung carcinoma, non-small cell lung carcinoma, squamous cell carcinoma, lung adenocarcinoma, large cell carcinoma, mesothelioma, lung neuroendocrine carcinoma, smoking-associated lung cancer), prostate cancer, colorectal cancer (e.g., rectal adenocarcinoma), endometrial cancer (e.g., endometrioid endometrial cancer, endometrial adenocarcinoma), breast cancer (e.g., hormone-receptor-positive breast cancer, triple-negative breast cancer, neuroendodrine carcinoma of the breast), skin cancer (e.g., melanoma, cutaneous squamous cell carcinoma, basal cell carcinoma, large squamous cell carcinoma), gallbladder cancer, liposarcoma (e.g., dedifferentiated liposarcoma, myxoid liposarcoma), pheochromocytoma, myoepithelial carcinoma, urothelial carcinoma, spermatocytic seminoma, stomach cancer, head and neck cancer (e.g., head and neck (squamous) carcinoma, head and neck adenoid cystic adenocarcinoma), brain cancer (e.g., glialneural tumors, glioma, neuroblastoma, glioblastoma, pilocytic astrocytoma, Rosette forming glioneural tumor, dysembryoplastic neuroepithelial tumor, anaplastic astrocytoma, medulloblastoma, ganglioglioma, oligodendroglioma), malignant peripheral nerve sheath tumor, sarcoma (e.g., soft tissue sarcoma (e.g., leiomyosarcoma), osteosarcoma), esophageal cancer (e.g., esophageal adenocarcinoma), lymphoma, bladder cancer (e.g., bladder urothelial (transition cell) carcinoma), cervical cancer (e.g., cervical squamous cell carcinoma, cervical adenocarcinoma), fallopian tube cancer (e.g., fallopian tube carcinoma), ovarian cancer (e.g., ovarian serous cancer, ovarian mucinous carcinoma), cholangiocarcinoma, adenoid cystic carcinoma, pancreatic cancer (e.g., pancreatic exocrine carcinoma, pancreatic ductal adenocarcinoma, pancreatic cancer intraepithelial neoplasia), salivary gland cancer (e.g., pleomorphic salivary gland adenocarcinoma, salivary adenoid cystic cancer), oral cancer (e.g., oral squamous cell carcinoma), uterine cancer, gastric or stomach cancer (e.g., gastric adenocarcinoma), gastrointestinal stromal tumors, myeloma (e.g., multiple myeloma), lymphoepithelioma, anal cancer (e.g., anal squamous cell carcinoma), prostate cancer (e.g., prostate adenocarcinoma), renal cell carcinoma, thymic cancer, gastroesophogeal junction adenocarcinoma, testicular cancer, rhabdomyosarcoma (e.g., alveolar rhabdomyosarcoma, embryonic rhabomyosarcoma), renal papillary carcinoma, liver cancer (e.g., hepatocellular carcinoma, intrahepatic cholangiocarcinoma), carcinoid, myeloid proliferative disorders (also called myeloid proliferative neoplasms (MPN); e.g., 8pll myeloproliferative syndrome (EMS, also called stem cell leukemia/lymphoma), acute myeloid leukemia (AML), chronic myeloid leukemia (CML)), lymphoma (e.g., T-cell lymphoma, T-lymphoblastic lymphoma, acute lymphoblastic leukemia (ALL), B-cell lymphoma), myeloid and lymphoid neoplasms, chronic neutrophilic leukemia, phosphaturic mesenchymal tumor, thyroid cancer (e.g. anaplastic thyroid carcinoma), or biliary duct cancer.

In some embodiments of any of the methods or uses described herein, the cancer (e.g., FGFR-associated cancer) is selected from the group of: acute lymphoblastic leukemia (ALL), acute myeloid leukemia (AML), cancer in adolescents, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, unknown primary carcinoma, cardiac tumors, cervical cancer, childhood cancers, chordoma, chronic lymphocytic leukemia (CLL), chronic myelogenous leukemia (CML), chronic myeloproliferative neoplasms, neoplasms by site, neoplasms, colon cancer, colorectal cancer, craniopharyngioma, cutaneous T-cell lymphoma, cutaneous angiosarcoma, bile duct cancer, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, extrahepatic bile duct cancer, eye cancer, fallopian tube cancer, fibrous histiocytoma of bone, gallbladder cancer, gastric cancer, gastrointestinal carcinoid tumor, gastrointestinal stromal tumors (GIST), germ cell tumor, gestational trophoblastic disease, glioma, hairy cell tumor, hairy cell leukemia, head and neck cancer, thoracic neoplasms, head and neck neoplasms, CNS tumor, primary CNS tumor, heart cancer, hepatocellular cancer, histiocytosis, Hodgkin's lymphoma, hypopharyngeal cancer, intraocular melanoma, islet cell tumors, pancreatic neuroendocrine tumors, Kaposi sarcoma, kidney cancer, Langerhans cell histiocytosis, laryngeal cancer, leukemia, lip and oral cavity cancer, liver cancer, lung cancer, lymphoma, macroglobulinemia, malignant fibrous histiocytoma of bone, osteocarcinoma, melanoma, Merkel cell carcinoma, mesothelioma, metastatic squamous neck cancer, midline tract carcinoma, mouth cancer, multiple endocrine neoplasia syndromes, multiple myeloma, mycosis fungoides, myelodysplastic syndromes, myelodysplastic/myeloproliferative neoplasms, neoplasms by site, neoplasms, myelogenous leukemia, myeloid leukemia, multiple myeloma, myeloproliferative neoplasms, nasal cavity and para nasal sinus cancer, nasopharyngeal cancer, neuroblastoma, non-Hodgkin's lymphoma, non-small cell lung cancer, lung neoplasm, pulmonary cancer, pulmonary neoplasms, respiratory tract neoplasms, bronchogenic carcinoma, bronchial neoplasms, oral cancer, oral cavity cancer, lip cancer, oropharyngeal cancer, osteosarcoma, ovarian cancer, pancreatic cancer, papillomatosis, paraganglioma, para nasal sinus and nasal cavity cancer, parathyroid cancer, penile cancer, pharyngeal cancer, pheochromosytoma, pituitary cancer, plasma cell neoplasm, pleuropulmonary blastoma, pregnancy-associated breast cancer, primary central nervous system lymphoma, primary peritoneal cancer, prostate cancer, rectal cancer, colon cancer, colonic neoplasms, renal cell cancer, retinoblastoma, rhabdomyosarcoma, salivary gland cancer, sarcoma, Sezary syndrome, skin cancer, Spitz tumors, small cell lung cancer, small intestine cancer, soft tissue sarcoma, squamous cell carcinoma, squamous neck cancer, stomach cancer, T-cell lymphoma, testicular cancer, throat cancer, thymoma and thymic carcinoma, thyroid cancer, transitional cell cancer of the renal pelvis and ureter, unknown primary carcinoma, urethral cancer, uterine cancer, uterine sarcoma, vaginal cancer, vulvar cancer, and Wilms' tumor.

In some embodiments, a hematological cancer (e.g., hematological cancers that are FGFR associated cancers) is selected from the group consisting of leukemias, lymphomas (non-Hodgkin's lymphoma), Hodgkin's disease (also called Hodgkin's lymphoma), and myeloma, for instance, acute lymphocytic leukemia (ALL), acute myeloid leukemia (AML), acute promyelocytic leukemia (APL), chronic lymphocytic leukemia (CLL), chronic myeloid leukemia (CML), chronic myelomonocytic leukemia (CMML), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult Tcell ALL, AML with trilineage myelodysplasia (AML/TMDS), mixed lineage leukemia (MLL), myelodysplastic syndromes (MDSs), myeloproliferative disorders (MPD), and multiple myeloma (MM).

Additional examples of hematological cancers include myeloproliferative disorders (MPD) such as polycythemia vera (PV), essential thrombocytopenia (ET) and idiopathic primary myelofibrosis (IMF/IPF/PMF). In some embodiments, the hematological cancer (e.g., the hematological cancer that is a FGFR-associated cancer) is AML or CMML.

In some embodiments, the cancer (e.g., the FGFR-associated cancer) is a solid tumor. Examples of solid tumors (e.g., solid tumors that are FGFR-associated cancers) include, for example, lung cancer (e.g., lung adenocarcinoma, non-small-cell lung carcinoma, squamous cell lung cancer), bladder cancer, colorectal cancer, brain cancer, testicular cancer, bile duct cancer cervical cancer, prostate cancer, and sparmatocytic seminomas. See, for example, Turner and Grose, Nat. Rev. Cancer, 10(2):116-129, 2010.

In some embodiments, the cancer is selected from the group consisting of bladder cancer, brain cancer, breast cancer, cholangiocarcinoma, head and neck cancer, lung cancer, multiple myeloma, rhabdomyosarcoma, urethral cancer, and uterine cancer. In some embodiments, the cancer is selected from the group consisting of lung cancer, breast cancer, and brain cancer. In some embodiments, a FGFRl-associated cancer is selected from the group consisting of lung cancer, breast cancer, and brain cancer. In some embodiments, the cancer is selected from the group consisting of breast cancer, uterine cancer, cholangiocarcinoma, and lung cancer. In some embodiments, a FGFR2-associated cancer is selected from the group consisting of breast cancer, uterine cancer, cholangiocarcinoma, and lung cancer. In some embodiments, the cancer is selected from the group consisting of lung cancer, bladder cancer, urethral cancer, multiple myeloma, and head and neck cancer. In some embodiments, a FGFR3-associated cancer is selected from the group consisting of lung cancer, bladder cancer, urethral cancer, multiple myeloma, and head and neck cancer. In some embodiments, the cancer is selected from lung cancer, rhabdomyosarcoma, and breast cancer. In some embodiments, a FGFR4-associated cancer is selected from lung cancer, rhabdomyosarcoma, and breast cancer.

In some aspects, the compounds of the disclosure are useful in treating cancers associated with amplification or overexpression of FGFR1, for example, Breast cancer or carcinoma (e.g., hormone receptor-positive breast cancer, ductal carcinoma in situ (breast)), pancreatic ductal adenocarcinoma, pancreatic exocrine carcinoma, smoking-associated lung cancer, small cell lung cancer, lung adenocarcinoma, non-small cell lung cancer, squamous cell lung cancer or carcinoma, prostate cancer or carcinoma, ovarian cancer, fallopian tube carcinoma, bladder cancer, rhabdomyosarcoma, head and neck carcinoma (e.g., head and neck squamous cell carcinoma), esophageal cancer (e.g., esophageal squamous cell carcinoma), sarcoma (e.g., osteosarcoma), hepatocellular carcinoma, renal cell carcinoma, colorectal cancer (e.g., colorectal adenocarcinoma), prostate cancer, salivary gland tumors, glioblastoma multiforme, urinary bladder cancer, urothelial carcinoma, carcinoma of unknown primary, squamous non-lung tumors, gastric cancer, gastroesophageal junction carcinoma, adenoid cystic carcinoma, anal squamous cell carcinoma, oral squamous cell carcinoma, cholangiocarcinoma, hemangioendothelioma, leiomyosarcoma, melanoma, neuroendocrine carcinoma, squamous cell carcinoma, uterine carcinosarcoma.

In some aspects, the compounds of the disclosure are useful in treating cancers associated with amplification of FGFR2, for example, Gastric cancer, gastroesophageal junction adenocarcinoma, breast cancer (e.g., triple negative breast cancer), colon cancer, colorectal cancer (e.g., colorectal adenocarcinoma), urothelial cancer, bladder adenocarcinoma, carcinoma of unknown primary, cholangiocarcinoma, endometrial cancer, endometrial adenocarcinoma, esophageal adenocarcinoma, gallbladder carcinoma, ovarian cancer, fallopian tube carcinoma, pancreatic exocrine carcinoma, sarcoma, squamous cell carcinoma. In some aspects, the compounds of the disclosure are useful in treating cancers associated with overexpression of FGFR2, for example, Myxoid lipocarcinoma, rectal cancer, renal cell carcinoma, breast cancer.

In some aspects, the compounds of the disclosure are useful in treating cancers associated with upregulation of activity of FGFR3, for example, Colorectal cancer, hepatocellular carcinoma, pancreatic exocrine carcinoma. In some aspects, the compounds of the disclosure are useful in treating cancers associated with overexpression of activity of FGFR3, for example, Multiple myeloma, thyroid carcinoma. In some aspects, the compounds of the disclosure are useful in treating cancers associated with amplification of activity of FGFR3, for example, Bladder cancer and salivary adenoid cystic cancer, urothelial cancer, breast cancer, carcinoid, carcinoma of unknown primary, colorectal cancer (e.g., colorectal adenocarcinoma), gallbladder carcinoma, gastric cancer, gastroesophageal junction adenocarcinoma, glioma, mesothelioma, non-small cell lung carcinoma, small cell lung cancer, ovarian cancer, fallopian tube carcinoma, pancreatic exocrine carcinoma.

In some aspects, the compounds of the disclosure are useful in treating cancers associated with amplification of FGFR4, for example, Rhabdomyosarcoma, prostate cancer or carcinoma, breast cancer, urothelial cancer, carcinoid, carcinoma of unknown primary, esophageal adenocarcinoma, head and neck carcinoma, hepatocellular carcinoma, non-small cell lung carcinoma, ovarian cancer, fallopian tube carcinoma, peritoneal carcinoma, renal cell carcinoma.

In some aspects, the compounds of the disclosure are useful in treating cancers associated with upregulation of activity of FGFR4, for example, Colorectal cancer, hepatocellular carcinoma, adrenal carcinoma, breast cancer.

In some aspects, the compounds of the disclosure are useful in treating cancers associated with overexpression of activity of FGFR4, for example, Pancreatic intraepithelial neoplasia, and pancreatic ductal adenocarcinoma.

In some aspects, the compounds of the disclosure are more selective for an FGFR kinase over another kinase that is not an FGFR kinase. For example, the compounds of the disclosure are at least 3-fold more selective for an FGFR kinase over another kinase that is not an FGFR kinase. In some aspects, the compounds of the disclosure are at least 10, 20, 30, 40, 50, 60, 70, 80, 90, 200, 300, 400, 500, 600, 700, 800, 900, or at least 1000 fold more selective for an FGFR kinase over another kinase that is not an FGFR kinase. Kinases that are not FGFR kinases include, for example, KDR kinase and Aurora B kinase.

In some embodiments, the compounds of the disclosure exhibit brain and/or central nervous system (CNS) penetrance. Such compounds are capable of crossing the blood brain barrier and inhibiting a FGFR kinase in the brain and/or other CNS structures. In some embodiments, the compounds provided herein are capable of crossing the blood brain barrier in a therapeutically effective amount. For example, treatment of a subject with cancer (e.g., a FGFR-associated cancer such as a FGFR-associated brain or CNS cancer) can include administration (e.g., oral administration) of the compound to the subject. In some such embodiments, the compounds provided herein are useful for treating a primary brain tumor or metastatic brain tumor. For example, a FGFR-associated primary brain tumor or metastatic brain tumor.

In some embodiments, the compounds of the disclosure, exhibit one or more of high GI absorption, low clearance, and low potential for drug-drug interactions.

In some aspects, compounds of the disclosure can be used for treating a subject diagnosed with (or identified as having) a FGFR-associated disease or disorder (e.g., a FGFR-associated cancer) that include administering to the subject a therapeutically effective amount of a compound of the disclosure. Also provided herein are methods for treating a subject identified or diagnosed as having a FGFR-associated disease or disorder (e.g., a FGFR-associated cancer) that include administering to the subject a therapeutically effective amount of a compound of the disclosure. In some embodiments, the subject that has been identified or diagnosed as having a FGFR-associated disease or disorder (e.g., a FGFR-associated cancer) through the use of a regulatory agency-approved, e.g., FDA-approved test or assay for identifying dysregulation of a FGFR gene, a FGFR kinase, or expression or activity or level of any of the same, in a subject or a biopsy sample from the subject or by performing any of the non-limiting examples of assays described herein. In some embodiments, the test or assay is provided as a kit. In some embodiments, the FGFR-associated disease or disorder is a FGFR-associated cancer. For example, the FGFR-associated cancer can be a cancer that includes one or more FGFR inhibitor resistance mutations.

Also provided are methods for treating a disease or disorder in a subject in need thereof, the method comprising: (a) detecting a FGFR-associated disease or disorder in the subject; and (b) administering to the subject a therapeutically effective amount of a compound of the disclosure. Some embodiments of these methods further include administering to the subject an additional therapy or therapeutic agent (e.g., a second FGFR inhibitor, a second compound of the disclosure, or an immunotherapy. In some embodiments, the subject was previously treated with a first FGFR inhibitor or previously treated with another treatment. In some embodiments, the subject is determined to have a FGFR-associated disease or disorder through the use of a regulatory agency-approved, e.g., FDA approved test or assay for identifying dysregulation of a FGFR gene, a FGFR kinase, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: (a) detecting a FGFR-associated cancer in the subject; and (b) administering to the subject a therapeutically effective amount of a compound of the disclosure. Some embodiments of these methods further include administering to the subject an additional therapy or therapeutic agent (e.g., a second FGFR inhibitor, a second compound of the disclosure, or an immunotherapy). In some embodiments, the subject was previously treated with a first FGFR inhibitor or previously treated with another anticancer treatment, e.g., at least partial resection of the tumor or radiation therapy. In some embodiments, the subject is determined to have a FGFR-associated cancer through the use of a regulatory agency-approved, e.g., FDA-approved test or assay for identifying dysregulation of a FGFR gene, a FGFR kinase, or expression or activity or level of any of the same, in a subject or a biopsy sample from the subject or by performing any of the non-limiting examples of assays described herein. In some embodiments, the test or assay is provided as a kit. In some embodiments, the cancer is a FGFR associated cancer. For example, the FGFR-associated cancer can be a cancer that includes one or more FGFR inhibitor resistance mutations. In some embodiments, the cancer is a FGFR associated cancer. For example, the FGFR-associated cancer can be a cancer that includes one or more FGFR activating mutations.

Also provided are methods of treating a subject that include performing an assay on a sample obtained from the subject to determine whether the subject has a dysregulation of a FGFR gene, a FGFR kinase, or expression or activity or level of any of the same, and administering (e.g., specifically or selectively administering) a therapeutically effective amount of a compound of the disclosure or pharmaceutically acceptable salt or solvate thereof to the subject determined to have a dysregulation of a FGFR gene, a FGFR kinase, or expression or activity or level of any of the same. Some embodiments of these methods further include administering to the subject an additional therapy or therapeutic agent (e.g., a second FGFR inhibitor, a second compound of the disclosure, or immunotherapy). In some embodiments of these methods, the subject was previously treated with a first FGFR inhibitor or previously treated with another anticancer treatment, e.g., at least partial resection of a tumor or radiation therapy. In some embodiments, the subject is a subject suspected of having a FGFR-associated disease or disorder (e.g., a FGFR-associated cancer), a subject presenting with one or more symptoms of a FGFR-associated disease or disorder (e.g., a FGFR-associated cancer), or a subject having an elevated risk of developing a FGFR-associated disease or disorder (e.g., a FGFR-associated cancer). In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art. In some embodiments, the dysregulation of a FGFR gene, a FGFR kinase, or expression or activity or level of any of the same includes one or more FGFR inhibitor resistance mutations.

Also provided herein are methods of selecting a treatment for a subject, wherein the methods include a step of performing an assay on a sample obtained from the subject to determine whether the subject has a dysregulation of a FGFR gene, a FGFR kinase, or expression or activity or level of any of the same (e.g., one or more FGFR inhibitor resistance mutations), and identifying or diagnosing a subject determined to have a dysregulation of a FGFR gene, a FGFR kinase, or expression or activity or level of any of the same, as having a FGFR-associated cancer. Some embodiments further include administering the selected treatment to the subject identified or diagnosed as having a FGFR-associated cancer. For example, in some embodiments, the selected treatment can include administration of a therapeutically effective amount of a compound of the disclosure to the subject identified or diagnosed as having a FGFR-associated cancer. In some embodiments, the assay is an in vitro assay. For example, an assay that utilizes the next generation sequencing, immunohistochemistry, or break apart FISH analysis. In some embodiments, the assay is a regulatory agency-approved, e.g., FDA-approved, kit. In some embodiments, the assay is a liquid biopsy.

Also provided herein are methods of treating a FGFR-associated cancer in a subject that include (a) administering one or more (e.g., two or more, three or more, four or more, five or more, or ten or more) doses of a first FGFR kinase inhibitor to a subject identified or diagnosed as having a FGFR associated cancer (e.g., any of the types of FGFR-associated cancers described herein) (e.g., identified or diagnosed as having a FGFR-associated cancer using any of the exemplary methods described herein or known in the art); (b) after step (a), determining a level of circulating tumor DNA in a biological sample (e.g., a biological sample comprising blood, serum, or plasma) obtained from the subject; (c) administering a therapeutically effective amount of a second FGFR inhibitor or a compound of the disclosure as a monotherapy or in conjunction with an additional therapy or therapeutic agent to a subject identified as having about the same or an elevated level of circulating tumor DNA as compared to a reference level of circulating tumor DNA (e.g., any of the reference levels of circulating tumor DNA described herein). In some examples of these methods, the reference level of circulating tumor DNA is a level of circulating tumor DNA in a biological sample obtained from the subject prior to step (a). Some embodiments of these methods further include determining the level of circulating tumor DNA in the biological sample obtained from the subject prior to step (a). In some examples of these methods, the reference level of circulating tumor DNA is a threshold level of circulating tumor DNA (e.g., an average level of circulating tumor DNA in a population of subjects having a similar FGFR-associated cancer and having a similar stage of the FGFR-associated cancer, but receiving a non-effective treatment or a placebo, or not yet receiving therapeutic treatment, or a level of circulating tumor DNA in a subject having a similar FGFR-associated cancer and having a similar stage of the FGFR-associated cancer, but receiving a non-effective treatment or a placebo, or not yet receiving therapeutic treatment). In some examples of these methods, the first FGFR inhibitor is: ARQ-087, ASP5878, AZD4547, B-701, BAY1179470, BAY1187982, BGJ398, brivanib, Debio-1347, dovitinib, E7090, erdafitinib, FPA144, HMPL-453, INCB054828, lenvatinib, lucitanib, LY3076226, MAX-40279, nintedanib, orantinib, pemigatinib, ponatinib, PRN1371, rogaratinib, sulfatinib, TAS-120 or RLY-4008.

Compounds of the disclosure can also be administered with additional therapy or therapeutic agents. In some aspects, the additional therapy or therapeutic agent includes one or more of radiation therapy, a chemotherapeutic agent (e.g., any of the exemplary chemotherapeutic agents described herein or known in the art), a checkpoint inhibitor (e.g., any of the exemplary checkpoint inhibitors described herein or known in the art), surgery (e.g., at least partial resection of the tumor), and one or more other kinase inhibitors (e.g., any of the kinase inhibitors described herein or known in the art).

Compounds of the disclosure may also be useful as adjuvants to cancer treatment, that is, they can be used in combination with one or more additional therapies or therapeutic agents, for example a chemotherapeutic agent that works by the same or by a different mechanism of action. In some embodiments, a compound of the disclosure can be used prior to administration of an additional therapeutic agent or additional therapy. For example, a subject in need thereof can be administered one or more doses of a compound of the disclosure for a period of time and then under go at least partial resection of the tumor. In some embodiments, the treatment with one or more doses of a compound of the disclosure reduces the size of the tumor (e.g., the tumor burden) prior to the at least partial resection of the tumor. In some embodiments, a subject has a cancer (e.g., a locally advanced or metastatic tumor) that is refractory or intolerant to standard therapy (e.g., administration of a chemotherapeutic agent, such as a first FGFR inhibitor or a multikinase inhibitor, immunotherapy, radiation, or a platinum-based agent (e.g., cisplatin)). In some embodiments, a subject has a cancer (e.g., a locally advanced or metastatic tumor) that is refractory or intolerant to prior therapy (e.g., administration of a chemotherapeutic agent, such as a first FGFR inhibitor or a multikinase inhibitor, immunotherapy, radiation, or a platinum-based agent (e.g., cisplatin)).

In some embodiments of any the methods described herein, the compound of the disclosure is administered in combination with a therapeutically effective amount of at least one additional therapeutic agent selected from one or more additional therapies or therapeutic (e.g., chemotherapeutic) agents.

Non-limiting examples of additional therapeutic agents include: other FGFR-targeted therapeutic agents (i.e. a first or second FGFR kinase inhibitor), other kinase inhibitors (e.g., receptor tyrosine kinase targeted therapeutic agents (e.g., Trk inhibitors or EGFR inhibitors)), signal transduction pathway inhibitors, checkpoint inhibitors, modulators of the apoptosis pathway (e.g. obataclax); cytotoxic chemotherapeutics, angiogenesis-targeted therapies, immune-targeted agents, including immunotherapy, and radiotherapy.

Also provided herein are methods of treating a disease or disorder, comprising administering to a subject in need thereof a pharmaceutical combination for treating the disease or disorder which comprises (a) a compound of the disclosure, (b) an additional therapeutic agent, and (c) optionally at least one pharmaceutically acceptable carrier for simultaneous, separate or sequential use for the treatment of the disease or disorder, wherein the amounts of the compound of the disclosure and the additional therapeutic agent are together effective in treating the disease or disorder. In some embodiments, the compound of the disclosure, and the additional therapeutic agent are administered simultaneously as separate dosages. In some embodiments, the compound of the disclosure, and the additional therapeutic agent are administered as separate dosages sequentially in any order, in jointly therapeutically effective amounts, e.g. in daily or intermittently dosages. In some embodiments, the compound of the disclosure, and the additional therapeutic agent are administered simultaneously as a combined dosage. In some embodiments, the disease or disorder is a FGFR-associated disease or disorder. In some embodiments, the subject has been administered one or more doses of a compound of the disclosure, prior to administration of the pharmaceutical composition.

In some embodiments, the treatment period is at least 7 days (e.g., at least or about 8 days, at least or about 9 days, at least or about 10 days, at least or about 11 days, at least or about 12 days, at least or about 13 days, at least or about 14 days, at least or about 15 days, at least or about 16 days, at least or about 17 days, at least or about 18 days, at least or about 19 days, at least or about 20 days, at least or about 21 days, at least or about 22 days, at least or about 23 days, at least or about 24 days, at least or about 25 days, at least or about 26 days, at least or about 27 days, at least or about 28 days, at least or about 29 days, or at least or about 30 days).

In some embodiments, the treatment period is at least 21 days (e.g., at least or about 22 days, at least or about 23 days, at least or about 24 days, at least or about 25 days, at least or about 26 days, at least or about 27 days, at least or about 28 days, at least or about 29 days, at least or about 30 days, at least or about 31 days, at least or about 32 days, at least or about 33 days, at least or about 34 days, at least or about 35 days, at least or about 36 days, at least or about 37 days, at least or about 38 days, at least or about 39 days, or at least or about 40 days).

Also provided herein are pharmaceutical compositions that contain, as the active ingredient, a compound of the disclosure, in combination with one or more pharmaceutically acceptable carriers (excipients). In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is formulated as a tablet or capsule.

The compositions comprising a compound of the disclosure can be formulated in a unit dosage form, each dosage containing from about 5 to about 1,000 mg (1 g), more usually about 100 mg to about 500 mg, of the active ingredient. The term “unit dosage form” refers to physically discrete units for human subjects and other subjects, each unit containing a predetermined quantity of active material (i.e., a compound of the disclosure) to produce the desired therapeutic effect, with a suitable pharmaceutical excipient.

In some embodiments, the compositions provided herein contain from about 5 mg to about 50 mg of the active ingredient, i.e., the compound of the disclosure. One having ordinary skill in the art will appreciate that this embodies compounds or compositions containing about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg of the active ingredient. In some embodiments, the compositions provided herein contain from about 50 mg to about 500 mg of the active ingredient. One having ordinary skill in the art will appreciate that this embodies compounds or compositions containing about 50 mg to about 100 mg, about 100 mg to about 150 mg, about 150 mg to about 200 mg, about 200 mg to about 250 mg, about 250 mg to about 300 mg, about 350 mg to about 400 mg, or about 450 mg to about 500 mg of the active ingredient. In some embodiments, the compositions provided herein contain from about 500 mg to about 1,000 mg of the active ingredient. One having ordinary skill in the art will appreciate that this embodies compounds or compositions containing about 500 mg to about 550 mg, about 550 mg to about 600 mg, about 600 mg to about 650 mg, about 650 mg to about 700 mg, about 700 mg to about 750 mg, about 750 mg to about 800 mg, about 800 mg to about 850 mg, about 850 mg to about 900 mg, about 900 mg to about 950 mg, or about 950 mg to about 1,000 mg of the active ingredient.

The active compound may be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual subject, the severity of the subject's symptoms, and the like.

In some embodiments, the compounds provided herein can be administered in an amount ranging from about 1 mg/kg to about 100 mg/kg. In some embodiments, the compound provided herein can be administered in an amount of about 1 mg/kg to about 20 mg/kg, about 5 mg/kg to about 50 mg/kg, about 10 mg/kg to about 40 mg/kg, about 15 mg/kg to about 45 mg/kg, about 20 mg/kg to about 60 mg/kg, or about 40 mg/kg to about 70 mg/kg. For example, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg. In some embodiments, such administration can be once-daily or twice-daily (BID) administration.

In several embodiments, not only can the compounds of the disclosure be used for therapy in patients with FGFR mutations (either point mutations or various fusions) to provide superior benefits, but in situations in which these mutations are likely to arise (such as in erdafitinib and/or infigratinib (BGJ398), pemigatinib, or TAS-120 therapy), where numerous activating and resistance mutations recur in patients, it may be especially advantageous. In several embodiments, the compounds of the disclosure can be used as a therapeutic intervention in patients bearing these mutations, either in combination with a pan-FGFR inhibitor or as a monotherapy where genomic testing supports mutations for which the compounds of the disclosure are active.

In several embodiments, a method of treating a cancer is provided. In several embodiments, the method of treating cancer includes administering one or more compounds of the disclosure. In several embodiments, the method comprises, in response to a determination of the presence of a FGFR mutant polypeptide or a FGFR mutant polynucleotide in a sample from the subject, administering to the subject an effective amount of compounds of the disclosure. This can thereby treat the cancer in the subject. In several embodiments, the FGFR mutant is one of the ones disclosed herein and/or an activating mutant (including a point mutation or FGFR fusion).

In several embodiments, the method of treating cancer includes administering one or more compounds of the disclosure to a patient who is suspected of having a cancer or being at risk of having a cancer. In several embodiments, the method comprises administering to the subject an effective amount of a compound of the disclosure, this can be done with or without a diagnosis or analysis of the subject's kinases (including whether or not the kinases are wild-type or mutant).

In several embodiments, the FGFR mutants are fusions that can be caused by chromosomal translocations in cancers. These translocations can lead to fusion proteins that exert their oncogenic effects through overexpression or growth factor independent activation of an otherwise normal gene or creation of a chimeric gene in which parts of two genes are fused together. Fusions of FGFR genes with other genes or parts of genes have been found most commonly in FGFR2 and FGFR3. The most common fusion partner reported for FGFR3 is TACC3 (Transforming Acidic Coiled-Coil Containing Protein).

In several embodiments, mutations in FGFR are polyclonal. Thus, when the FGFR or FGFR-fusion driven cancer metastasizes, the individual metastases can have distinct mutational patterns in the FGFR kinase domain. For example, a patient with distinct liver metastases can have a gatekeeper mutation in a subset of the metastases but not necessarily in all of them at the time of treatment or biopsy. The presence of the founding mutation from the primary tumor i.e. a FGFR fusion would likely remain in all patients. In several embodiments, it is that founding mutation that is targeted by any one or more of the methods provided herein. In several embodiments, both the founding mutation and other mutations are targeted by any one or more of the methods provided herein. In several embodiments, only the later mutations are targeted by one or more of the methods provided herein. In several embodiments, the method of any of the methods provided herein can be one where a compound of the disclosure is administered in an amount adequate to treat a tumor in a subject who has metastasized, and wherein the tumor that is being treated is the primary tumor. In several embodiments, any of the methods provided herein can use an adequate amount of a compound of the disclosure to treat a subset of the tumors in a subject. For example, the subset can include or focus on the tumors with a founding mutation (the primary tumor(s)). Thus, in several embodiments, the therapy need not be directed to, or include an amount of the disclosure to treat every tumor, but just a subset of the tumors (for example the primary tumors with the founding mutation). In several embodiments, the treated tumor is not the primary tumor, but may be a metastases with a detectable resistance or activating mutation not found in the primary tumor. In several embodiments, the method comprises administering the disclosure in an amount adequate to treat a tumor in a subject who has metastasized, and wherein the treated tumor is not the primary tumor, and wherein the treated tumor is a metastases with a detectable resistance or activating mutation not found in the primary tumor.

In several embodiments, the compound can be used to treat subjects with other types of mutations in FGFR, including allosteric mutations, such as FGFR3 S249C.

In several embodiments, a method of treating a cancer is provided, the method comprises, in response to a determination of the presence of a FGFR activating mutation in a subject, administering to the subject an effective amount of a compound of the disclosure thereby treating the cancer in the subject, wherein the FGFR activating mutation is a driver in a non-fused cancer. In several embodiments, an effective amount of a compound of the disclosure is an amount that reduces the activity of the FGFR mutant to a level that is adequate to provide some treatment to the subject, for example, by reducing one or more symptoms. In several embodiments, the activity of the mutant FGFR is reduced by a compound of the disclosure to near, or lower than, wild-type activity. In several embodiments, the activity for the FGFR mutant, when a compound of the disclosure is administered, is reduced to 500, 400, 300, 200, 190, 180, 170, 160, 150, 140, 130, 120, 110, 105, 104, 103, 102, 101, 100, 95, 90, or lower percent of the activity of wild-type FGFR.

In several embodiments, a method of treating cancer in a subject in need thereof is provided. The method comprises administering an inhibitor of FGFR kinase activity to a subject determined to have a genetic fusion of FGFR and a second gene, wherein the inhibitor of FGFR is at least as effective against the genetic fusion of FGFR, as it is against a wild-type FGFR kinase. In several embodiments, the inhibitor can be a compound of the disclosure. In several embodiments, the inhibitor of FGFR kinase activity is at least 5, 10, 20, 30, 40, 50, 60, 70, 80, 90, 100, 110, 120, 130, 140, 150, 200, 300, 400, 500, 1000, 5000, or 10,000% more potent on the fused FGFR as on the wild-type FGFR. In several embodiments, a compound of the disclosure is at least 1.1, 2, 5, 10, 20, 30, 40, 50, 60, 70, 80, 90, or 100-fold as potent for the mutant as it is for wild-type. In several embodiments, the IC50 for a compound of the disclosure is 0.5, 0.1, 0.05, or 0.01% as large for the mutant FGFR as it is for wild type (that is, the numerical value for the IC50 is lower for the mutant).

In several embodiments, the IC₅₀ of a compound of the disclosure to the FGFR mutant or mutation is no higher than about 100 nM (e.g., it is at least as good in potency as 100 nM). In several embodiments, the IC₅₀ of a compound of the disclosure to the FGFR mutant or mutation is no higher than about 10 nM (e.g., it is at least as good in potency as 10 nM). In several embodiments, the IC₅₀ of a compound of the disclosure to the FGFR mutant or mutation is no higher than single digit nM (e.g., it is at least as good in potency as single digit nM). In several embodiments, the IC₅₀ of a compound of the disclosure to the FGFR mutant or mutation is at least as effective for the FGFR mutant or mutation as it is for a wild type FGFR.

In several embodiments of the method, the subject has been (or is still) on a multi-targeted kinase inhibitor (“MKI”) or a targeted FGFR inhibitor. While on the MKI or the targeted FGFR inhibitor, the subjects tumor become resistant to the prior MKI or the targeted FGFR inhibitor. At this point, one can administer a compound of the disclosure. In the alternative, one can determine if the subject now has a tumor that has a FGFR mutation in it (such as amino acid changes that result in resistance to the prior therapy). If the subject does have a tumor with a mutation, one can then dose the subject with a compound of the disclosure.

In several embodiments, the method of using a compound of the disclosure can be directed to treating a variety of cancers or cancer generically. In several embodiments, the cancer is one or more of: urothelial carcinoma, breast carcinoma, endometrial cancer, endometrial adenocarcinoma, ovarian carcinoma, primary glioma, cholangiocarcinoma, gastric adenocarcinoma, non-small cell lung carcinoma, pancreatic exocrine carcinoma, oral, prostate, bladder, colorectal carcinoma, renal cell carcinoma, neuroendocrine carcinoma, myeloproliferative neoplasms, head and neck (squamous), melanoma, leiomyosarcoma, and/or sarcomas. In several embodiments, the subject has an intrahepatic cholangiocarcinoma. This list denotes some, but not all of the FGFR mutant related cancers. In several embodiments, the cancer can include any of the previous options and/or any of the following: urothelial carcinoma, breast carcinoma, endometrial adenocarcinoma, ovarian carcinoma, primary glioma, cholangiocarcinoma, gastric adenocarcinoma, non-small cell lung carcinoma, pancreatic exocrine carcinoma, oral, prostate, bladder, colorectal carcinoma, renal cell carcinoma, neuroendocrine carcinoma, myeloproliferative neoplasms, head and neck (squamous), melanoma, leiomyosarcoma, and/or sarcomas. In several embodiments, the subject has an intrahepatic cholangiocarcinoma. In several embodiments, the cancer can include any of the previous options and/or any of the following: urothelial carcinoma, breast carcinoma, endometrial cancer, endometrial adenocarcinoma, ovarian carcinoma, primary glioma, cholangiocarcinoma, gastric adenocarcinoma, non-small cell lung carcinoma, pancreatic exocrine carcinoma, oral, prostate, bladder, colorectal carcinoma, renal cell carcinoma, neuroendocrine carcinoma, myeloproliferative neoplasms, head and neck (squamous), melanoma, leiomyosarcoma, and/or sarcomas.

### EXAMPLES

In some embodiments, the compounds of the disclosure are the compounds described in the Examples that follow.

General methods use to synthesize examples of the invention are exemplified with the following representative examples.

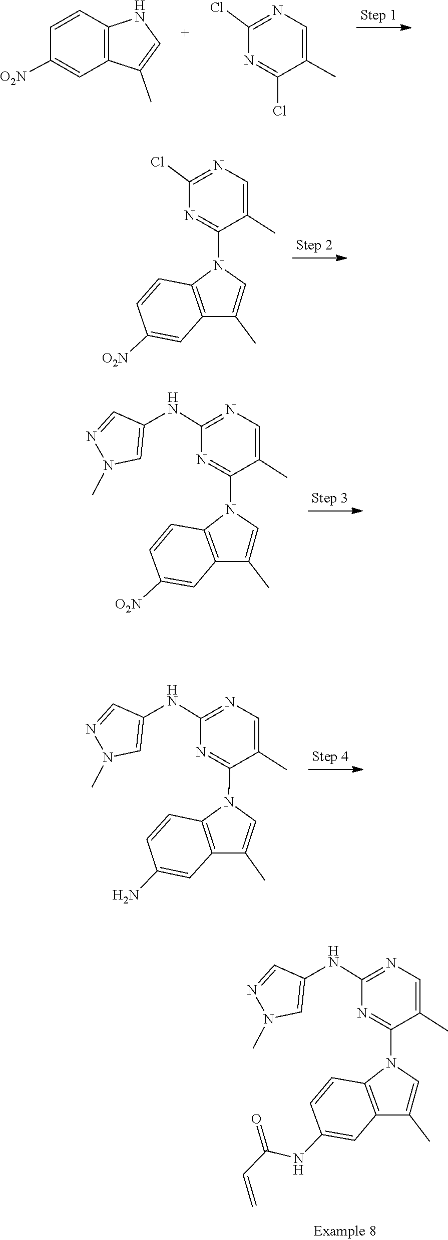

### Example 8. N-[3-Methyl-1-[5-methyl-2-[(1-methylpyrazol-4-yl)amino]pyrimidin-4-yl]indol-5-yl]prop-2-enamide

Step 1. 1-(2-Chloro-5-methyl-pyrimidin-4-yl)-3-methyl-5-nitro-indole. To a solution of 3-methyl-5-nitro-1H-indole (2.0 g, 11.4 mmol) in dry DMF (14 mL), cooled to 0° C. (ice bath), was added NaH (60.0%, 0.681 g, 17.0 mmol) portion wise. The resulting dark red suspension was stirred at 0° C. for 30 min and then slowly transferred to a solution of 2,4-dichloro-5-methyl-pyrimidine (2.22 g, 13.6 mmol) in dry DMF (6 ml). The reaction mixture was stirred at RT for 2 h. Water was added (20 ml) and the resulting precipitate was collected by filtration, washed with water, MeOH and dried in vacuo to afford a yellow solid (3.0 g, 75%). LCMS m/z=303 (M+1).

Step 2. 5-Methyl-4-(3-methyl-5-nitro-indol-1-yl)-N-(1-methylpyrazol-4-yl)pyrimidin-2-amine. A suspension of 1-(2-chloro-5-methyl-pyrimidin-4-yl)-3-methyl-5-nitro-indole (300 mg, 0.991 mmol) and 1-methylpyrazol-4-amine (115 mg, 1.19 mmol) in isopropanol (4.80 mL) was irradiated in a microwave reactor at 140° C. for 30 minutes. The reaction was irradiated for additional 30 min at 140° C. The precipitate that formed was collected to give a yellow solid (255 mg; 85%). LCMS m/z=364 (M+1)

Step 3. 3-Methyl-1-[5-methyl-2-[(1-methylpyrazol-4-yl)amino]pyrimidin-4-yl]indol-5-amine. To a solution of 5-methyl-4-(3-methyl-5-nitro-indol-1-yl)-N-(1-methylpyrazol-4-yl)pyrimidin-2-amine (250 mg, 0.605 mmol) in a mixture of ethanol (15 mL) and water (4 mL) was added ammonium chloride (64.8 mg, 1.21 mmol) and Fe (406 mg, 7.27 mmol). The resulting mixture was stirred at 80° C. for 2 h. The mixture was cooled to RT, diluted with DCM, filtered through celite and the residue washed with DCM and methanol. The organic layers were combined, washed with saturated NaHCO₃ solution (20 mL), dried over Na₂SO₄ and concentrated to give a solid (212 mg). LCMS m/z=334 (M+1).

Step 4. N-[3-Methyl-1-[5-methyl-2-[(1-methylpyrazol-4-yl)amino]pyrimidin-4-yl]indol-5-yl]prop-2-enamide. To an cooled solution of 3-methyl-1-[5-methyl-2-[(1-methylpyrazol-4-yl)amino]pyrimidin-4-yl]indol-5-amine (50 mg, 0.142 mmol) in dry THF (1.5 ml), was added diisopropylethylamine (0.0486 mL, 0.356 mmol) followed by acryloyl chloride (12.9 mg, 0.142 mmol). The resulting mixture was stirred at RT for 45 minutes. The reaction was diluted with saturated NaHCO₃ (10 ml) and extracted with DCM (3×10 ml). The organic layers were combined, dried over sodium sulfate and evaporated to give a brown solid. Trituration with MeOH and diethyl ether gave a white solid (20 mg; 36%). LCMS m/z=388 (M+1); ¹H NMR (500 MHz, DMSO-d6) δ: 10.14 (s, 1H), 9.45 (br s, 1H), 8.42 (s, 1H), 8.06 (d, J=1.5 Hz, 1H), 7.88-7.68 (m, 2H), 7.57-7.55 (m, 1H), 7.48 (s, 1H), 7.40 (dd, J=9.0, 2.0 Hz, 1H), 6.48 (dd, J=16.9, 10.2 Hz, 1H), 6.26 (dd, J=16.9, 2.0 Hz, 1H), 5.76-5.72 (m, 1H), 3.75 (br s, 3H), 2.29 (s, 3H), 2.21 (s, 3H).

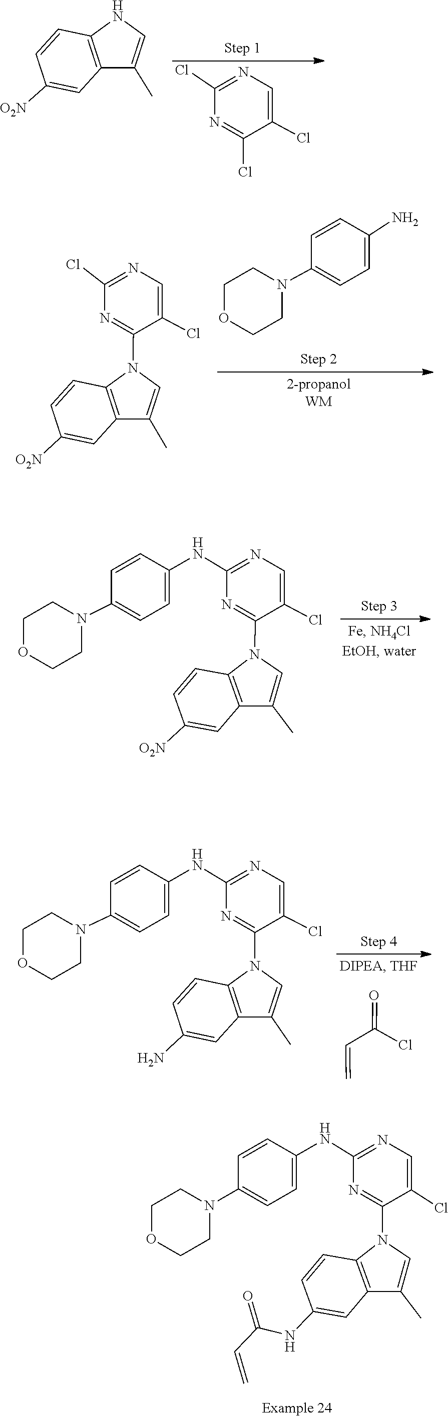

### Example 24. N-[3-methyl-1-[5-chloro-2-(4-morpholinoanilino)pyrimidin-4-yl]indol-5-yl]prop-2-enamide

Step 1. 1-(2,5-Dichloropyrimidin-4-yl)-3-methyl-5-nitro-indole. A solution of 3-methyl-5-nitro-1H-indole (467 mg, 2.7 mmol.) in DMF (5 mL) at 0° C. (ice bath), was added NaH (60% dispersion in mineral oil, 127 mg, 1.2 eq.) cautiously portion-wise. The suspension was stirred at ice bath temperature for 30 min. The resulting slurry was then transferred to a cold (0° C.) solution 2,4,5-trichloropyrimidine (583 mg, 3.2 mmol., 1.20 eq.) in DMF (5 mL) and stirred for 30 min. Upon completion, water (10 mL) was added cautiously with stirring. The precipitate was collected, washed with EtOAc (3 mL) and dried in vacuo to afford a yellow powder (725 mg, 83%). L CMS m/z=323, 325 (M+1, 1 C₁).

Step 2. 5-Chloro-4-(3-methyl-5-nitro-indol-1-yl)-N-(4-morpholinophenyl) pyrimidin-2-amine. A suspension of 1-(2,5-dichloropyrimidin-4-yl)-3-methyl-5-nitro-indole (300 mg, 0.928 mmol) and 4-morpholinoaniline (199 mg, 1.11 mmol) in 2-propanol (10 mL) was heated in MW at 140° C. for 60 minutes. The precipitate formed in the reaction was filtered off, washed with a small amount of MeOH and dried in vacuo to give a yellow solid (371 mg, 86%). LCMS m/z=465 (M+1).

Step 3. 1-[5-Chloro-2-(4-morpholinoanilino)pyrimidin-4-yl]-3-methyl-indol-5-amine. A suspension of 5-chloro-4-(3-methyl-5-nitro-indol-1-yl)-N-(4-morpholinophenyl)pyrimidin-2-amine (371 mg, 0.798 mmol), iron (535 mg, 9.58 mmol) and ammonium chloride (171 mg, 3.19 mmol) in EtOH (30 mL) and water (5 mL) was stirred at 85° C. for 2 h. Upon complete conversion, the reaction mixture was filtrated through celite and concentrated to dryness. The remaining residue was suspended in DCM, washed with saturated NaHCO₃ solution, water and brine. The organic layer was separated, dried over MgSO₄ and evaporated to dryness to give a yellow solid (325 mg, 94%). LCMS m/z=435 (M+1).

Step 4. N-[1-[5-chloro-2-(4-morpholinoanilino)pyrimidin-4-yl]-3-methyl-indol-5-yl]prop-2-enamide. To a solution of 1-[5-chloro-2-(4-morpholinoanilino)pyrimidin-4-yl]-3-methyl-indol-5-amine (325 mg, 0.590 mmol) in dry THF (10 mL) was added DIPEA (0.161 mL, 1.18 mmol) followed by acryloyl chloride (0.0528 mL, 0.649 mmol). The resulting mixture was stirred at RT for 30 min. The reaction was then concentrated in vacuo. The residue was dissolved in DCM, washed with saturated NaHCO₃ solution and brine, then dried over Na₂SO₄. After the DCM was evaporated the crude product was purified by silica gel column chromatography (DCM:MeOH=20:1) to give a yellow solid (152 mg; 53%). LCMS m/z=489 (M+1); ¹H NMR (500 MHz, DMSO-d6) δ: 10.17 (s, 1H), 9.74 (s, 1H), 8.60 (s, 1H), 8.06 (d, J=1.5 Hz, 1H), 7.84 (br s, 1H), 7.70 (s, 1H), 7.55 (d, J=9.2 Hz, 2H), 7.41 (d, J=8.9 Hz, 1H), 6.88 (br d, J=8.9 Hz, 2H), 6.47 (dd, J=17.1, 10.1 Hz, 1H), 6.27 (dd, J=16.9, 2.0 Hz, 1H), 5.73-5.77 (m, 1H), 3.73 (t, J=4.6 Hz, 4H), 3.03 (t, J=4.9 Hz, 4H), 2.28 (s, 3H).

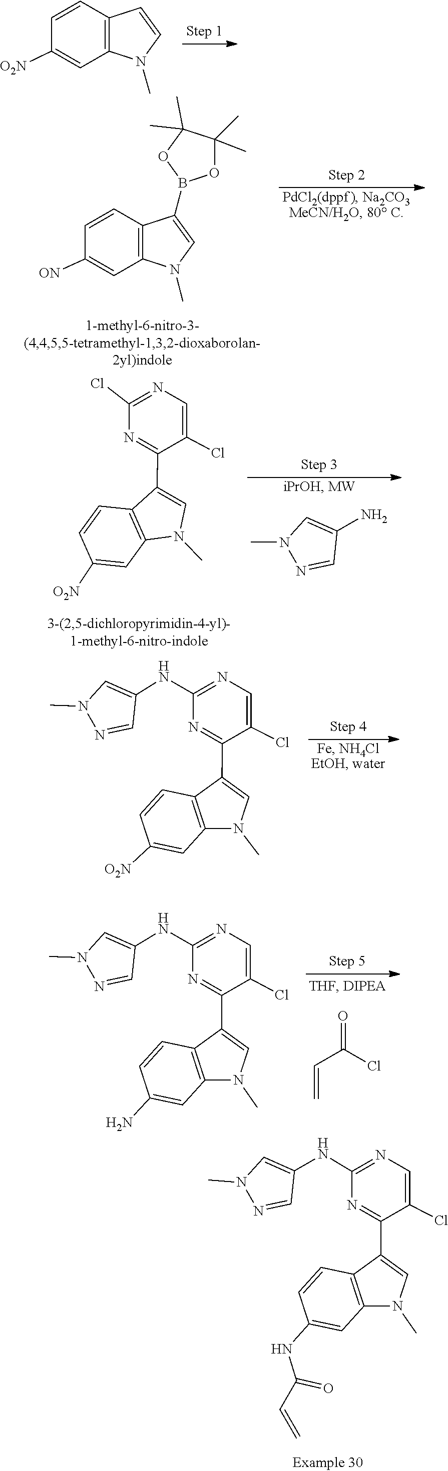

### Example 30. N-[3-[5-Chloro-2-[(1-methylpyrazol-4-yl)amino]pyrimidin-4-yl]-1-methyl-indol-6-yl]prop-2-enamide

Step 1. 1-Methyl-6-nitro-3-(4,4,5,5-tetramethyl-1,3,2-dioxaborolan-2-yl)indole. A 100 ml reaction tube was charged boron tribromide (1M in DCM; 45.4 mL, 45.4 mmol), DCM (20 ml) and 2,6-lutidine (5.29, 45.4 mmol) in this order. The solution was cooled in an ice bath for 10 minutes and then a solution of 1-methyl-6-nitro-indole (2.0 g, 11.4 mmol) in DCM (10 ml) was added dropwise during 10 minutes. The reaction mixture was allowed to reach RT. After stirring at RT for 1 hour, the reaction was cooled to 0° C. and a solution of pinacol (5.37 g, 45.4 mmol) in N,N-diisopropylethylamine (29.7 ml, 170.3 mmol) was added dropwise during 35 min. The resulting mixture was allowed to reach RT and stirred for 1 h. The reaction was then diluted with DCM and washed with NaHCO₃ solution, water and brine. The organic extract was concentrated in vacuo and the crude material was purified by silica gel chromatography (EtOAc/CyHex=1/3) to give a green solid (1.65 g, 49%). LCMS m/z=303 (M+1).

Step 2. 3-(2,5-Dichloropyrimidin-4-yl)-1-methyl-6-nitro-indole. A suspension of 1-methyl-6-nitro-3-(4,4,5,5-tetramethyl-1,3,2-dioxaborolan-2-yl)indole (530 mg, 1.75 mmol), 2,4,5-trichloropyrimidine (221 μL, 1.93 mmol, 1.1 eq) and sodium carbonate (0.37 g, 3.51 mmol, 2 eq) in acetonitrile (12 mL) and water (6 mL) was bubbled with argon for 10 minutes. Tetrakis(triphenylphosphine)palladium(0) (0.10 mg, 0.09 mmol, 0.05 eq) was added and the resulting mixture was irradiated in MW reactor for 60 minutes at 80° C. The reaction mixture was poured into water, the precipitate was collected by filtration and dried in vacuo to give a yellow solid (250 mg, 44%). LCMS m/z=323 (M+1); ¹H NMR (500 MHz, DMSO-d6) δ 9.06 (s, 1H) 8.83 (s, 1H) 8.63 (d, J=9.1 Hz, 1H) 8.61 (d, J=1.8 Hz, 1H) 8.18 (dd, J=8.8, 2.1 Hz, 1H) 4.09 (s, 3H).

Step 3. 5-Chloro-4-(1-methyl-6-nitro-indol-3-yl)-N-(1-methylpyrazol-4-yl)pyrimidin-2-amine. A suspension of 1-methylpyrazol-4-amine, (25.0 mg, 0.26 mmol) and 3-(2,5-dichloropyrimidin-4-yl)-1-methyl-6-nitro-indole (70.0 mg, 0.22 mmol) in isopropanol (1.0 mL) was heated in a microwave reactor at 140° C. for 90 min. After cooling to 25° C., the product was collected by filtration, washed with MeOH and dried in vacuo to give a yellow solid (70 mg; 55%). LCMS m/z=384 (M+1).

Step 4. 3-[5-Chloro-2-[(1-methylpyrazol-4-yl)amino]pyrimidin-4-yl]-1-methyl-indol-6-amine. To a suspension of 5-chloro-4-(1-methyl-6-nitro-indol-3-yl)-N-(1-methylpyrazol-4-yl)pyrimidin-2-amine (70.0 mg, 0.182 mmol) in EtOH/water (3 mL/1 mL) was added iron (122 mg, 2.19 mmol), followed by ammonium chloride (19.5 mg, 0.36 mmol). The mixture was stirred at reflux for 1 h. Upon complete conversion, the reaction mixture was diluted with DCM (30 mL), stirred at RT for 5 min, filtered through celite and concentrated in vacuo to dryness to give a yellow solid (98 mg; 65%). LCMS m/z=354 (M+1).

Step 5. N-[3-[5-Chloro-2-[(1-methylpyrazol-4-yl)amino]pyrimidin-4-yl]-1-methyl-indol-6-yl]prop-2-enamide. To a solution of 3-[5-chloro-2-[(1-methylpyrazol-4-yl)amino]pyrimidin-4-yl]-1-methyl-indol-6-amine (98 mg, 0.21 mmol) in dry THF (5 mL) was added DIPEA (72 μL, 0.41 mmol) followed by acryloyl chloride (17 μL, 0.21 mmol). The resulting mixture was stirred at RT for 5 min. Upon completion, the reaction mixture was evaporated to dryness. The product was purified by prep HPLC to give a yellow solid (21 mg; 25%). LCMS m/z=408 (M+1); ¹H NMR (500 MHz, DMSO-d6) δ: 9.97 (br s, 1H), 9.09 (br s, 1H), 8.46 (br d, 1H, J=8.5 Hz), 8.39 (s, 1H), 8.35 (s, 1H), 8.08 (d, 1H, J=0.9 Hz), 7.84 (s, 1H), 7.53 (s, 1H), 7.32 (dd, 1H, J=1.7, 8.7 Hz), 6.50 (dd, 1H, J=10.2, 16.9 Hz), 6.29 (dd, 1H, J=2.0, 16.9 Hz), 5.73 (dd, 1H, J=1.8, 10.4 Hz), 3.87 (s, 3H), 3.82 (s, 3H).

### Example 32. N-[1-[5-cyano-2-[(1-methylpyrazol-4-yl)amino]pyrimidin-4-yl]-3-methyl-indol-5-yl]prop-2-enamide

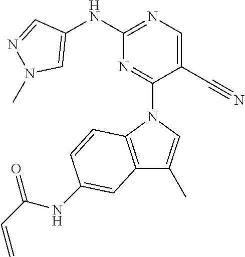 [SMILES: C=CC(=O)Nc1ccc2c(c1)c(C)cn2-c1nc(Nc2cnn(C)c2)ncc1C#N]

Step 1. 4-(3-Methyl-5-nitro-indol-1-yl)-2-[(1-methylpyrazol-4-yl)amino]pyrimidine-5-carbonitrile. A suspension of 2-chloro-4-(3-methyl-5-nitro-indol-1-yl)pyrimidine-5-carbonitrile, (300 mg, 0.96 mmol) and 1-methylpyrazol-4-amine (111 mg, 1.15 mmol) in isopropanol (4.5 mL) was irradiated in microwave reactor at 140° C. for 30 minutes. After cooling to RT, the precipitate was collected and washed with MeOH to afford the title product as mixture of regioisomers (316 mg). LCMS m/z=375 (M+1).

Step 2. 4-(5-Amino-3-methyl-indol-1-yl)-2-[(1-methylpyrazol-4-yl)amino]pyrimidine-5-carbonitrile. A suspension of 4-(3-methyl-5-nitro-indol-1-yl)-2-[(1-methylpyrazol-4-yl)amino]pyrimidine-5-carbonitrile (310 mg, 0.83 mmol), iron (555 mg, 9.94 mmol) and ammonium chloride (88.6 mg, 1.66 mmol) in EtOH (12 mL) and water (4.5 mL) was heated at reflux for 1.5 h. Then the mixture was cooled to RT, DCM (15 mL) was added and the mixture stirred for 5 min. The solution was filtered to remove the precipitate, which was washed with DCM (10 mL) and MeOH (10 mL). The filtrate layers were separated, the organic phase was dried over Na₂SO₄ and the solvent evaporated to give a brown solid (266 mg; 68%). LCMS m/z=345 (M+1).

Step 3. N-[1-[5-cyano-2-[(1-methylpyrazol-4-yl)amino]pyrimidin-4-yl]-3-methyl-indol-5-yl]prop-2-enamide. To the stirred solution of the product from step 2 (266 mg, 0.58 mmol) and DIPEA (202 μL, 1.16 mmol) in dry THF (15 mL) at 0° C. was added acryloyl chloride (46.8 μL, 0.58 mmol). The reaction mixture was stirred at RT for 5 min, quenched with water (0.1 mL) and then concentrated to give a crude product. The product was purified by prep HPLC to give a light brown solid (30 mg; 13%). LCMS m/z=399 (M+1); ¹H NMR (600 MHz, DMSO-d6) δ: 10.48-10.42 (m, 2H), 10.24 (d, J=7.3 Hz, 2H), 8.89 (s, 1H), 8.85 (s, 1H), 8.48 (d, J=9.0 Hz, 1H), 8.09 (d, J=1.8 Hz, 1H), 8.07 (d, J=1.8 Hz, 1H), 8.00 (d, J=8.8 Hz, 1H), 7.97 (s, 1H), 7.92 (d, J=1.1 Hz, 1H), 7.85 (s, 1H), 7.80 (d, J=1.3 Hz, 1H), 7.58 (d, J=3.7 Hz, 2H), 7.55 (dd, J=2.0, 9.0 Hz, 1H), 7.44 (dd, J=2.1, 8.9 Hz, 1H), 6.51-6.45 (m, 2H), 6.30-6.26 (m, 2H), 5.78-5.75 (m, 2H), 3.85 (s, 3H), 3.80 (s, 3H), 2.31-2.28 (m, 6H).

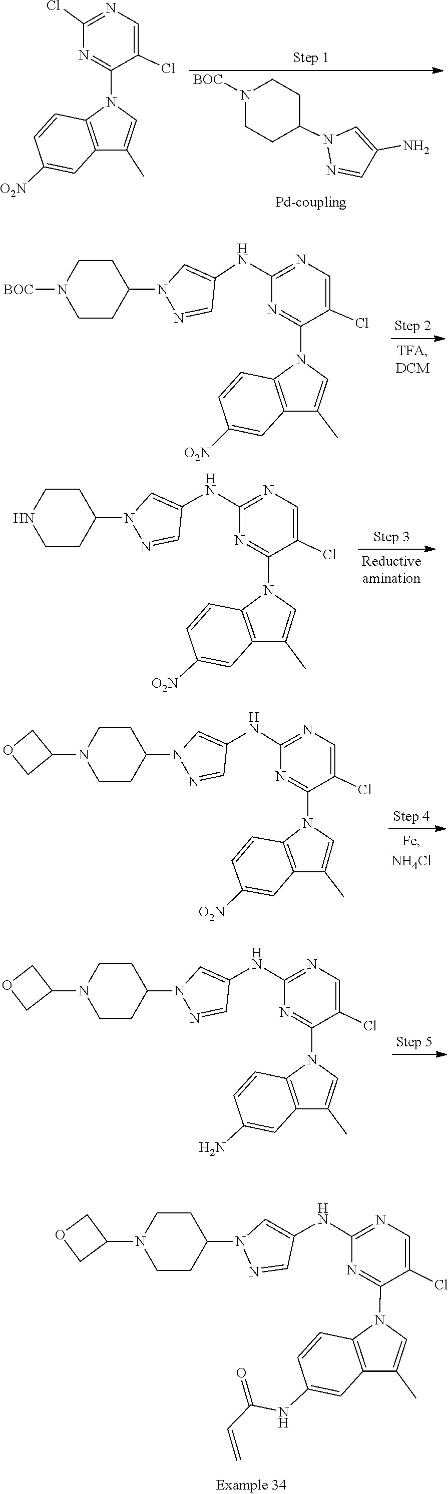

### Example 34. N-[1-[5-Chloro-2-[[1-[1-(oxetan-3-yl)-4-piperidyl]pyrazol-4-yl]amino]pyrimidin-4-yl]-3-methyl-indol-5-yl]prop-2-enamide

Step 1. tert-Butyl 4-[4-[[5-chloro-4-(3-methyl-5-nitro-indol-1-yl)pyrimidin-2-yl]amino]pyrazol-1-yl]piperidine-1-carboxylate. A suspension of 1-(2,5-dichloropyrimidin-4-yl)-3-methyl-5-nitro-indole (1 g, 3.09 mmol), tert-butyl 4-(4-aminopyrazol-1-yl)piperidine-1-carboxylate (907 mg, 3.40 mmol, 1.1 eq) cesium carbonate (2 g, 6.19 mmol, 2 eq) and 4,5-bis(diphenylphosphino)-9,9-dimethylxanthene (183 mg, 0.309 mmol, 0.1 eq) in dry dioxane (15 mL) was degassed with Argon for 5 min and then palladium(II)acetate (56 mg, 0.248 mmol, 0.08 eq) was added. The reaction mixture was heated in MW at 80° C. for 45 min. After cooling to RT, the reaction mixture was filtered through celite and washed with DCM. The solvent was concentrated to give a brown solid (1.95 g). This material was used directly in the next step. LCMS m/z=551 (M+1).

Step 2. 5-Chloro-4-(3-methyl-5-nitro-indol-1-yl)-N-[1-(4-piperidyl)pyrazol-4-yl]pyrimidin-2-amine. A solution of the product step 1 (1.95 g, 3.53 mmol) in DCM (15 ml) was added trifluoroacetic acid (5.40 ml, 70.52 mmol, 20 eq). The resulting mixture was stirred at RT for 30 min and then put on a conditioned SCX colum (20 g). The column was washed with MeOH (5×30 ml) and then with 2N NH₃/MeOH (50 ml) to give a yellow foam (1.27 g; 80%). LCMS m/z=453 (M+1).

Step 3. 5-Chloro-4-(3-methyl-5-nitro-indol-1-yl)-N-[1-[1-(oxetan-3-yl)-4-piperidyl]pyrazol-4-yl]pyrimidin-2-amine. To a solution of 5-chloro-4-(3-methyl-5-nitro-indol-1-yl)-N-[1-(4-piperidyl)pyrazol-4-yl]pyrimidin-2-amine (250 mg, 0.552 mmol) and oxetan-3-one (35 μl, 0.552 mmol, 1 eq) in THF (5 mL) was added followed by sodium cyanoborohydride (41.6 mg, 0.662 mmol, 1.2 eq). The resulting mixture was stirred at RT overnight. The reaction mixture was diluted with saturated NaHCO₃ solution (15 ml) and extraction with EtOAc (3×15 ml). The organic layer was washed with water (1×30 ml) and brine (1×30 ml) to give a solid (236 mg; 51%) that was used directly in the next step. LCMS m/z=509 (M+1).

Step 4. 1-[5-Chloro-2-[[1-[1-(oxetan-3-yl)-4-piperidyl]pyrazol-4-yl]amino]pyrimidin-4-yl]-3-methyl-indol-5-amine. A suspension of 5-chloro-4-(3-methyl-5-nitro-indol-1-yl)-N-[1-[1-(oxetan-3-yl)-4-piperidyl]pyrazol-4-yl]pyrimidin-2-amine (236 mg, 0.464 mmol), iron (310.7 mg, 5.56 mmol, 12 eq) and ammonium chloride (99.2 mg, 1.85 mmol, 4 eq) in EtOH (3 mL) and water (1.5 mL) was stirred at reflux overnight. Upon complete conversion, the reaction mixture was diluted with DCM (30 mL), stirred at RT for 5 min, filtered through celite and concentrated to give a yellow solid (204 mg; 92%). LCMS m/z=479 (M+1).

Step 5. N-[1-[5-chloro-2-[[1-[1-(oxetan-3-yl)-4-piperidyl]pyrazol-4-yl]amino]pyrimidin-4-yl]-3-methyl-indol-5-yl]prop-2-enamide. To a solution of the product from step 4 (204 mg, 0.426 mmol) in dry THF (1 ml), was added diisopropylethylamine (145 μl, 1.604 mmol, 2.5 eq) followed by prop-2-enoyl chloride (34 μl, 0.426 mmol, 1 eq). The resulting mixture was stirred at RT for 30 minutes. The reaction was quenched with water (0.1 mL) and concentrated. The product was purified by prep HPLC to give a yellow solid (36 mg; 16%). LCMS m/z=533 (M+1); ¹H NMR (DMSO-d6, 500 MHz) δ: 9.91 (br s, 1H), 9.56 (s, 1H), 8.56 (s, 1H), 7.97 (s, 1H), 7.87 (s, 1H), 7.82 (br d, J=8.8 Hz, 1H), 7.67 (s, 1H), 7.53 (s, 1H), 7.51 (d, J=9.3 Hz, 1H), 6.48 (dd, J=16.9, 10.2 Hz, 1H), 6.27 (dd, J=16.9, 1.6 Hz, 1H), 5.72 (dd, J=10.2, 1.6 Hz, 1H), 4.5 (t, J=6.41 Hz, 2H), 4.44 (t, J=5.9 Hz, 2H), 4.01-4.09 (m, 1H), 3.47 (quin, J=6.3 Hz, 1H), 2.72-2.82 (m, 2H), 2.31 (s, 3H), 2.00 (br t, J=10.3 Hz, 4H), 1.81-1.94 (m, 2H).

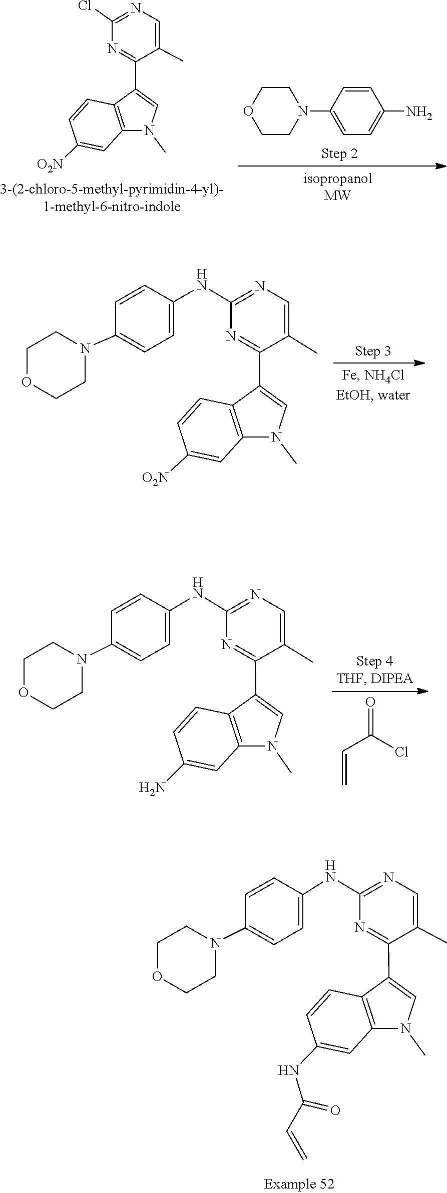

### Example 52. N-[1-Methyl-3-[5-methyl-2-(4-morpholinoanilino)pyrimidin-4-yl]indol-6-yl]prop-2-enamide

Step 1. 3-(2-Chloro-5-methyl-pyrimidin-4-yl)-1-methyl-6-nitro-indole. This intermediate was synthesized using the procedure for example 30 step 2 with 1-methyl-6-nitro-3-(4,4,5,5-tetramethyl-1,3,2-dioxaborolan-2-yl)indole and 2,4-dichloro-5-methyl-pyrimidine to give a solid. LCMS m/z=303 (M+1).

Step 2. 5-Methyl-4-(1-methyl-6-nitro-indol-3-yl)-N-(4-morpholinophenyl)pyrimidin-2-amine. A suspension of 4-morpholinoaniline (162.0 mg, 0.908 mmol) and 3-(2-chloro-5-methyl-pyrimidin-4-yl)-1-methyl-6-nitro-indole (125.0 mg, 0.413 mmol) in isopropanol (16.0 mL) was heated in a microwave reactor at 150° C. for 7 h. After cooling to 25° C., the reaction mixture was concentrated and the residue was purified by silica gel chromatography (DCM:MeOH=100:4) to give a yellow solid (25 mg; 14%). LCMS m/z=445.20 (M+1).

Step 3. 1-Methyl-3-[5-methyl-2-(4-morpholinoanilino)pyrimidin-4-yl]indol-6-amine. To a suspension of 5-methyl-4-(1-methyl-6-nitro-indol-3-yl)-N-(4-morpholinophenyl)pyrimidin-2-amine (25.0 mg, 0.0562 mmol) in EtOH/water (5.0 mL/0.75 mL in a 25 mL flask) was added iron (37.7 mg, 0.675 mmol), followed by ammonium chloride (12.0 mg, 0.225 mmol). The resulting mixture was stirred at 85° C. for 30 min. The reaction mixture was cooled to 25° C., diluted with DCM (10.0 mL), and the organic layer was filtered off of the brown residue deposited in the flask. The contents of the flask were rinsed with DCM (30 mL), and the organic layer decanted. The combined organic layers were dried over Na₂SO₄, filtered, and the solvent concentrated to give a yellow solid (23 mg; 100%). LCMS m/z=415.24 (M+1).

Step 4. N-[1-Methyl-3-[5-methyl-2-(4-morpholinoanilino)pyrimidin-4-yl]indol-6-yl]prop-2-enamide. To a solution of 1-methyl-3-[5-methyl-2-(4-morpholinoanilino)pyrimidin-4-yl]indol-6-amine (23.2 mg, 0.0560 mmol) in dry THF (4.0 mL) was added N,N-diisopropylethylamine (29.2 μL, 0.168 mmol), followed by acryloyl chloride (4.5 μL, 0.056 mmol). The reaction mixture was stirred at 25° C. for 10 min. The reaction mixture was then transferred to a separatory funnel containing distilled water, and the mixture extracted with DCM. The combined DCM layers were dried over Na₂SO₄. The solvent was concentrated and the residue was purified by preparative HPLC to give a white solid (4.6 mg; 20%). LCMS m/z=469.13 (M+1); ¹H NMR (DMSO-d6, 500 MHz) δ:10.22 (s, 1H), 9.03 (s, 1H), 8.56 (d, J=8.63 Hz, 1H), 8.20 (s, 1H), 8.15 (s, 1H), 8.00 (s, 1H), 7.67-7.63 (m, 2H), 7.21 (d, J=8.66 Hz, 1H), 6.90-6.86 (m, 2H), 6.50 (dd, J=10.19, 16.95 Hz, 1H), 6.28 (dd, J=1.72, 16.99 Hz, 1H), 5.76 (dd, J=1.51, 10.03 Hz, 1H), 3.86 (s, 3H), 3.76-3.73 (m, 4H), 3.05-3.02 (m, 4H), 2.36 (s, 3H).

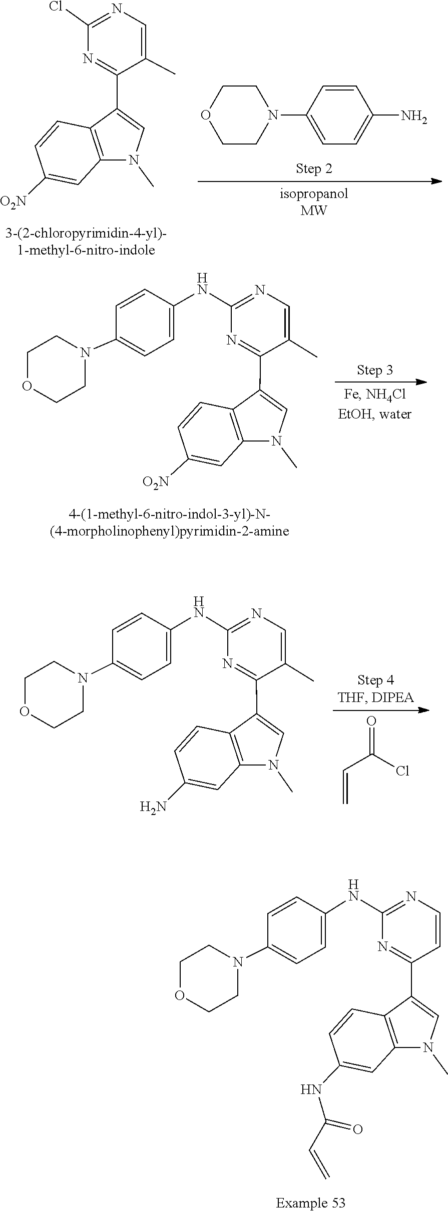

### Example 53. N-[1-Methyl-3-[2-(4-morpholino-anilino)pyrimidin-4-yl]indol-6-yl]prop-2-enamide

Step 1. 3-(2-Chloropyrimidin-4-yl)-1-methyl-6-nitro-indole. This intermediate was synthesized using the procedure for example 30 step 2 using 1-methyl-6-nitro-3-(4,4,5,5-tetramethyl-1,3,2-dioxaborolan-2-yl)indole and 2,4-dichloropyrimidine to give a yellow solid. LCMS m/z=307 (M+1).

Step 2. 4-(1-Methyl-6-nitro-indol-3-yl)-N-(4-morpholinophenyl)pyrimidin-2-amine. A suspension of 3-(2-chloropyrimidin-4-yl)-1-methyl-6-nitro-indole (356 mg, 1.23 mmol) and 4-morpholinoaniline, A20 (264 mg, 1.48 mmol) in isopropanol (12 mL) was irradiated in microwave reactor at 150° C. for 60 minutes. After cooling to RT the precipitate was collected by filtration and washed with iPrOH to give a brown solid (516 mg; 81%). LCMS m/z=431 (M+1).

Step 3. 1-Methyl-3-[2-(4-morpholinoanilino)pyrimidin-4-yl]indol-6-amine. A suspension of 4-(1-methyl-6-nitro-indol-3-yl)-N-(4-morpholinophenyl)pyrimidin-2-amine (516 mg, 1.20 mmol), iron (536 mg, 9.59 mmol) and ammonium chloride (128 mg, 2.40 mmol) in EtOH (20 mL) and water (9 mL) was heated at reflux for 5 min. The mixture was cooled to RT, diluted with DCM (20 mL) and stirred for 5 min. The precipitate was removed by filtration and washed with DCM (15 mL). The organic phase was washed with water and brine and then dried over Na₂SO₄. The solvent was concentrated to give a dark grey powder (425 mg; 83%). LCMS m/z=401 (M+1).

Step 4. N-[1-Methyl-3-[2-(4-morpholino-anilino)pyrimidin-4-yl]indol-6-yl]prop-2-enamide. A solution of 1-methyl-3-[2-(4-morpholinoanilino)pyrimidin-4-yl]indol-6-amine (405 mg, 0.85 mmol) with 4 Å molecular sieves (1.2 g) in EtOAc/DMF (40 mL/10 mL) at 0° C. was added TEA (947 μL, 6.80 mmol), acrylic acid (58.3 μL, 0.85 mmol) and T3P (50.0% wt. in ethyl acetate, 1.52 mL, 2.55 mmol). The mixture was stirred at RT for 10 min. An additional 50 μL of T3P added and stirring was continued for 5 min. The mixture was then transferred to a separatory funnel containing 100 mL of water, extracted and the layers separated. The aqueous phase was extracted with a DCM/MeOH mixture (10:1; 5×70 mL). The organic layers were washed with 5% aqueous LiCl (3×50 mL), combined, dried over Na₂SO₄ and evaporated to dryness. The product was purified by prep HPLC to give a yellow solid (31 mg). LCMS m/z=455 (M+1); ¹H NMR (DMSO-d6, 600 MHz) δ: 10.23 (s, 1H), 9.14 (s, 1H), 8.52 (br d, J=8.3 Hz, 1H), 8.27 (d, J=5.2 Hz, 1H), 8.23 (s, 1H), 8.15 (d, J=1.5 Hz, 1H), 7.66 (d, J=8.0 Hz, 2H), 7.25 (dd, J=8.6, 1.5 Hz, 1H), 7.10 (d, J=5.3 Hz, 1H), 6.93 (d, J=7.9 Hz, 2H), 6.50 (dd, J=16.9, 10.1 Hz, 1H), 6.28 (dd, J=17.0, 1.9 Hz, 1H), 5.74-5.78 (m, 1H), 3.83 (s, 3H), 3.72-3.79 (m, 4H), 3.03-3.09 (m, 4H).

### Example 54. N-[3-Methyl-1-[2-(4-morpholinoanilino)pyrimidin-4-yl]indol-5-yl]prop-2-enamide

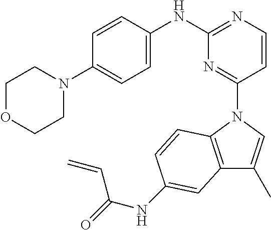 [SMILES: C=CC(=O)Nc1ccc2c(c1)c(C)cn2-c1ccnc(Nc2ccc(N3CCOCC3)cc2)n1]

Step 1. 4-(3-methyl-5-nitro-indol-1-yl)-N-(4-morpholinophenyl)pyrimidin-2-amine. A suspension of 1-(2-chloropyrimidin-4-yl)-3-methyl-5-nitro-indole (200 mg, 0.48 mmol) and 4-morpholinoaniline (104 mg, 0.58 mmol, 1.2 eq) in 2-propanol (4 mL) was heated in MW at 150° C. for 60 min. the mixture was concentrated and the residue purified by silica gel chromatography (DCM:MeOH=100:4) to give a white solid (152 mg). LCMS m/z=431 (M+1).

Step 2. 3-Methyl-1-[2-(4-morpholinoanilino)pyrimidin-4-yl]indol-5-amine. A suspension of 4-(3-methyl-5-nitro-indol-1-yl)-N-(4-morpholinophenyl)pyrimidin-2-amine (145 mg, 0.34 mmol), iron (226 mg, 4.04 mmol) and ammonium chloride (72 mg, 1.35 mmol) in EtOH (3 mL) and water (1.5 mL) was stirred at 85° C. for 2 h. The reaction mixture was diluted with DCM (15 ml), filtered and the solvent was evaporated in vacuo. The residue was suspended in water (50 ml) and extracted with DCM (3×30 ml). The combined organic layers were washed with water, brine and concentrated to give a solid (78 mg; 78%). LCMS m/z=401 (M+1).

Step 3. N-[3-methyl-1-[2-(4-morpholinoanilino)pyrimidin-4-yl]indol-5-yl]prop-2-enamide. To a solution of 3-methyl-1-[2-(4-morpholinoanilino)pyrimidin-4-yl]indol-5-amine (78 mg, 0.1916 mmol) in dry THF (1 mL) was added DIPEA (85 μl, 0.49 mmol) followed by acryloyl chloride (3 μl, 0.19 mmol). The resulting mixture was stirred at RT for 30 min. Reaction mixture was concentrated in vacuo. The residue was dissolved in DCM, washed with saturated NaHCO₃ and brine, dried over Na₂SO₄ and the solvent concentrated. The product was purified by silica gel column chromatography (DCM:MeOH=20:1) to give a yellow solid (35 mg; 45%). LCMS m/z=455 (M+1); ¹H NMR (500 MHz, DMSO-d6) δ: 10.17 (s, 1H), 9.39 (s, 1H), 8.69 (br s, 1H), 8.36 (d, J=5.8 Hz, 1H), 8.05 (d, J=1.8 Hz, 1H), 7.91 (s, 1H), 7.58 (br d, J=8.5 Hz, 2H), 7.42 (br d, J=8.2 Hz, 1H), 7.03 (d, J=5.8 Hz, 1H), 6.95 (d, J=9.1 Hz, 2H), 6.47 (dd, J=16.9, 10.2 Hz, 1H), 6.27 (dd, J=16.9, 1.9 Hz, 1H), 5.73-5.77 (m, 1H), 3.72-3.79 (m, 4H), 3.03-3.12 (m, 4H), 2.27 (s, 3H).

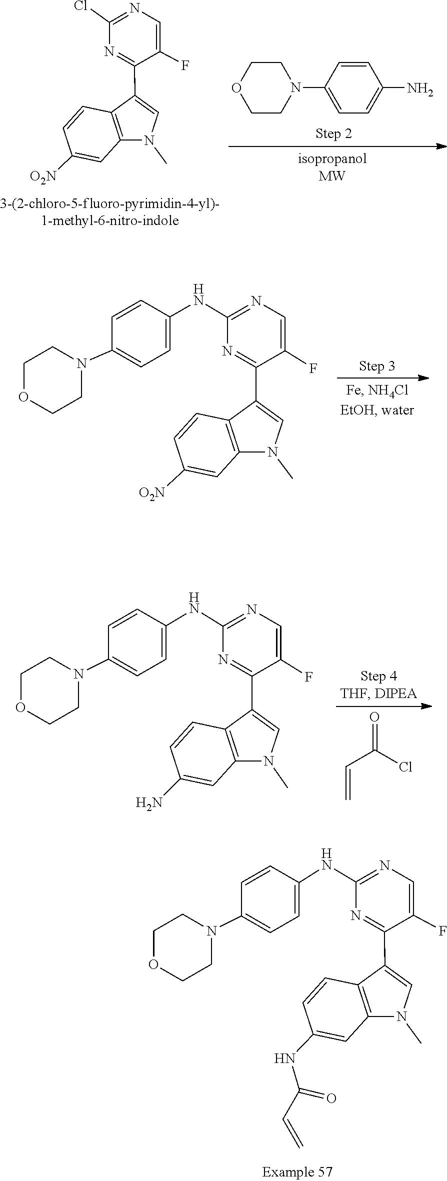

### Example 57. N-[3-[5-Fluoro-2-(4-morpholinoanilino)pyrimidin-4-yl]-1-methyl-indol-6-yl]prop-2-enamide

Step 1. 3-(2-Chloro-5-fluoro-pyrimidin-4-yl)-1-methyl-6-nitro-indole. This intermediate was synthesized using the procedure for example 30 step 2 with 1-methyl-6-nitro-3-(4,4,5,5-tetramethyl-1,3,2-dioxaborolan-2-yl)indole and 2,4-dichloro-5-fluoro-pyrimidine to give a yellow solid. LCMS m/z=307 (M+1).

Step 2. 5-Fluoro-4-(1-methyl-6-nitro-indol-3-yl)-N-(4-morpholinophenyl)pyrimidin-2-amine. A suspension of 3-(2-chloro-5-fluoro-pyrimidin-4-yl)-1-methyl-6-nitro-indole (240 mg, 0.783 mmol) and 4-morpholinoaniline (167 mg, 0.939 mmol) in isopropanol (5 mL) was heated in a microwave reactor at 150° C. for 1 h and then at 155° C. for 1 h. The reaction mixture was cooled to 25° C. The precipitate was collected by filtration, washed with isopropanol and dried to give a brown solid (321 mg; 92%). LCMS m/z=449.24 (M+1)

Step 3. 3-[5-Fluoro-2-(4-morpholinoanilino)pyrimidin-4-yl]-1-methyl-indol-6-amine. To a suspension of 5-fluoro-4-(1-methyl-6-nitro-indol-3-yl)-N-(4-morpholinophenyl)pyrimidin-2-amine (321 mg, 0.716 mmol) in EtOH/water (30.0 mL/7.0 mL) was added iron (480 mg, 8.59 mmol), followed by ammonium chloride (153 mg, 2.86 mmol). The resulting mixture was stirred at reflux (85° C. oil bath temperature) for 30 min. The reaction mixture was then cooled to 25° C., diluted with DCM, and the organic layer was filtered to remove a brown solid. The contents of the flask were rinsed with DCM/MeOH, and the organic layer filtered. The combined organic phases were dried over Na₂SO₄, filtered, and concentrated to give a brown solid (279 mg; 93%). LCMS m/z=419.20 (M+1).

Step 4. N-[3-[5-Fluoro-2-(4-morpholinoanilino)pyrimidin-4-yl]-1-methyl-indol-6-yl]prop-2-enamide. A solution of 3-[5-fluoro-2-(4-morpholinoanilino)pyrimidin-4-yl]-1-methyl-indol-6-amine (300 mg, 0.716 mmol) in dry THF (20.0 mL) was added N,N-diisopropylethylamine (374.0 μL, 2.15 mmol), followed by acryloyl chloride (57.9 μL, 0.716 mmol). The reaction mixture was stirred at 25° C. for 10 min. The reaction mixture was transferred to a separatory funnel containing distilled water, and the mixture extracted with DCM. The combined organic extracts were dried over Na₂SO₄, filtered, and concentrated to give a brown solid. The product was purified by preparative HPLC to give a white solid (125 mg; 37%). LCMS m/z=472.10 (M+1); ¹H NMR (DMSO-d6, 500 MHz) δ:10.26 (s, 1H), 9.29 (s, 1H), 8.65 (d, J=8.50 Hz, 1H), 8.36 (d, J=3.93 Hz, 1H), 8.19-8.17 (m, 2H), 7.64-7.59 (m, 2H), 7.30-7.26 (m, 1H), 7.00 (br s, 2H), 6.50 (dd, J=10.06, 16.90 Hz, 1H), 6.28 (dd, J=1.95, 16.90 Hz, 1H), 5.77 (dd, J=1.88, 10.03 Hz, 1H), 3.87 (s, 3H), 3.80-3.76 (m, 4H), 3.16-3.08 (m, 4H).

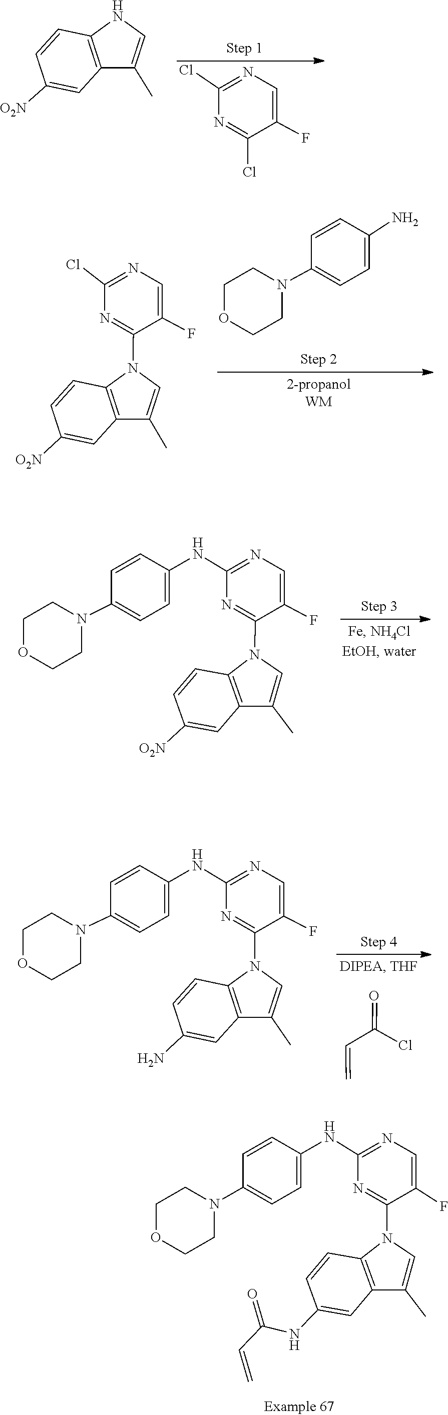

### Example 67. N-[1-[5-Fluoro-2-(4-morpholino-anilino)pyrimidin-4-yl]-3-methyl-indol-5-yl]prop-2-enamide

Step 1. 3-(2-Chloro-5-fluoro-pyrimidin-4-yl)-1-methyl-6-nitro-indole. This intermediate was synthesized using the procedure for example 8 step 1 with 3-methyl-5-nitro-1H-indole and 2,4-dichloro-5-fluoro-pyrimidine to give a solid. LCMS m/z=307 (M+1).

Step 2. 5-Fluoro-4-(3-methyl-5-nitro-indol-1-yl)-N-(4-morpholinophenyl)pyrimidin-2-amine. A suspension of 1-(2-chloro-5-fluoro-pyrimidin-4-yl)-3-methyl-5-nitro-indole (300 mg, 0.978 mmol) and 4-morpholinoaniline (213 mg, 1.174 mmol, 1.2 eq) in 2-propanol (10 mL) was heated in a MW at 150° C. for 3 h. The precipitate that formed in the reaction was filtered off, washed with a small amount of MeOH and dried in vacuo to give a brown solid (400 mg; 91%). LCMS m/z=449 (M+1).

Step 3. 1-[5-Fluoro-2-(4-morpholinoanilino)pyrimidin-4-yl]-3-methyl-indol-5-amine. A suspension of 5-fluoro-4-(3-methyl-5-nitro-indol-1-yl)-N-(4-morpholinophenyl)pyrimidin-2-amine (400 mg, 0.892 mmol), iron (598 mg, 10.704 mmol) and ammonium chloride (191 mg, 3.568 mmol) in EtOH (7 mL) and water (3.5 mL) was stirred at 85° C. for 3 h. The reaction mixture was then filtrated through celite and concentrated in vacuo to dryness. The residue was suspended in DCM, washed with saturated NaHCO₃ solution, water and brine. Organic layer was separated, dried over MgSO₄ and concentrated to give a brown solid (193 mg; 52%). LCMS m/z=419 (M+1).

Step 4. N-[1-[5-fluoro-2-(4-morpholinoanilino)pyrimidin-4-yl]-3-methyl-indol-5-yl]prop-2-enamide. A solution of 1-[5-fluoro-2-(4-morpholinoanilino)pyrimidin-4-yl]-3-methyl-indol-5-amine (190 mg, 0.454 mmol) in dry THF (2 mL) was added DIPEA (198 μl, 1.135 mmol) and acryloyl chloride (37 μl, 0.454 mmol). The resulting mixture was stirred at RT for 30 min and then concentrated. The residue was dissolved in DCM, washed with NaHCO₃ and brine, dried over Na₂SO₄ and evaporated. The product was purified by prep HPLC to give a yellow solid (25 mg; 12%). LCMS m/z=472 (M+1); ¹H NMR (300 MHz, DMSO-d6) δ: 10.20 (s, 1H), 9.50 (s, 1H), 8.53 (d, J=4.7 Hz, 1H), 8.32-8.48 (m, 1H), 8.06 (d, J=1.7 Hz, 1H), 7.68 (s, 1H), 7.55 (d, J=9.0 Hz, 2H), 7.44 (br d, J=8.8 Hz, 1H), 6.94 (br d, J=9.0 Hz, 2H), 6.47 (dd, J=16.9, 10.0 Hz, 1H), 6.26 (dd, J=16.9, 2.1 Hz, 1H), 5.72-5.78 (m, 1H), 3.69-3.80 (m, 4H), 2.96-3.18 (m, 4H), 2.26 (s, 3H).

### Example 146. N-[1-[2-[(1,5-dimethylpyrazol-4-yl)amino]-5-fluoro-pyrimidin-4-yl]-3-methyl-indol-5-yl]prop-2-enamide

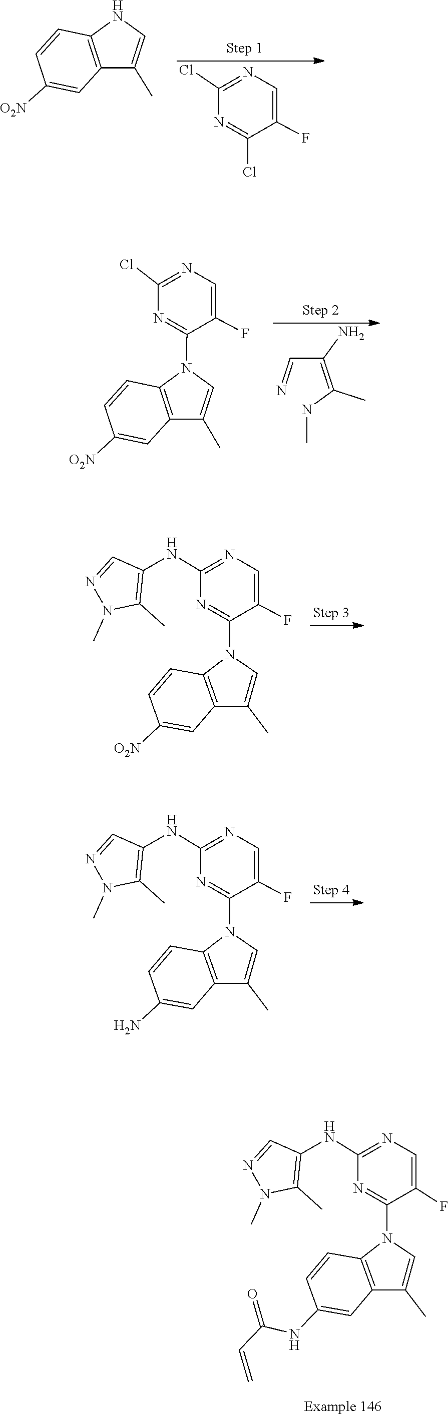

Step 1. 3-(2-Chloro-5-fluoro-pyrimidin-4-yl)-1-methyl-6-nitro-indole. This intermediate was synthesized using the procedure for example 8 step 1 with 3-methyl-5-nitro-1H-indole and 2,4-dichloro-5-fluoro-pyrimidine to give a solid. LCMS m/z=307 (M+1).

Step 2. N-(1,5-Dimethylpyrazol-4-yl)-5-fluoro-4-(3-methyl-5-nitro-indol-1-yl)pyrimidin-2-amine. To a suspension of 1,5-dimethylpyrazol-4-amine (99.7 mg, 0.897 mmol), 1-(2-chloro-5-fluoro-pyrimidin-4-yl)-3-methyl-5-nitro-indole (250 mg, 0.815 mmol) and potassium carbonate (225 mg, 1.63 mmol) in dry dioxane (6 mL) was bubbled in nitrogen for 10 minutes, then Xantphos (70.8 mg, 0.122 mmol) and Palladium (II) acetate (18.3 mg, 0.0815 mmol) were added. The resulting mixture was irradiated in MW at 80° C. for 45 min. The reaction was filtered and evaporated to dry. The residue was dissolved in DCM (50 mL) and washed with saturated NaHCO₃ solution (20 mL). the organic layer was dried over sodium sulfate and concentrated to give a crude solid, which was purified by silica gel chromatography (DCM:MeOH:NH₄OH=90:5:0.5; 0-50%) to give a solid (181 mg; 58%). LCMS m/z=382 (M+1).

Step 3. 1-[2-[(1,5-Dimethylpyrazol-4-yl)amino]-5-fluoro-pyrimidin-4-yl]-3-methyl-indol-5-amine. A suspension of N-(1,5-dimethylpyrazol-4-yl)-5-fluoro-4-(3-methyl-5-nitro-indol-1-yl)pyrimidin-2-amine (181 mg, 0.475 mmol), iron (318 mg, 5.70 mmol) and ammonium chloride (102 mg, 1.90 mmol) in EtOH (8 mL) and water (4 mL) was stirred at reflux (80° C.) for 0.5 h. Upon complete conversion, the reaction mixture was diluted with DCM, (50 ml), filtered and the layers were separated. The aqueous layer was extracted with DCM (2×30 ml). The organic layers were combined, dried over anhydrous Na₂SO₄, filtered and concentrated to give a yellow solid (165 mg, 99%). LCMS m/z=352 (M+1).

Step 4. N-[1-[2-[(1,5-dimethylpyrazol-4-yl)amino]-5-fluoro-pyrimidin-4-yl]-3-methyl-indol-5-yl]prop-2-enamide. To a solution of 1-[2-[(1,5-dimethylpyrazol-4-yl)amino]-5-fluoro-pyrimidin-4-yl]-3-methyl-indol-5-amine (280 mg, 0.797 mmol) in dry THF (12 ml), stirred at 0° C. was added DIPEA (0.272 mL, 1.99 mmol) followed by acryloyl chloride (72.1 mg, 0.797 mmol). The resulting mixture was stirred at RT for 20 min. Water was added and the reaction mixture was concentrated. The residue was dissolved in DCM (100 mL), washed with saturated NaHCO₃ solution and brine, then dried over Na₂SO₄ and concentrated. The crude solid was purified by silica gel chromatography (DCM/DCM:MeOH:NH₄OH=90:9:0.5=10/1) to give a yellow solid (167 mg, 52%). LCMS m/z=406 (M+1); ¹H NMR (500 MHz, DMSO-d6) δ: 9.88 (br s, 1H), 8.45 (s, 1H), 8.39 (d, J=4.9 Hz, 1H), 8.10 (br d, J=8.5 Hz, 1H), 7.98 (s, 1H), 7.63 (s, 1H), 7.42 (s, 1H), 7.38 (br d, J=8.9 Hz, 1H), 6.47 (dd, J=16.9, 10.2 Hz, 1H), 6.27 (br d, J=17.1 Hz, 1H), 5.72 (br d, J=10.1 Hz, 1H), 3.77 (s, 3H), 2.28 (s, 3H), 2.15 (s, 3H).

### Example 167. N-[1-[2-[(1,3-Dimethylpyrazol-4-yl)amino]-5-fluoro-pyrimidin-4-yl]-3-methyl-indol-5-yl]prop-2-enamide

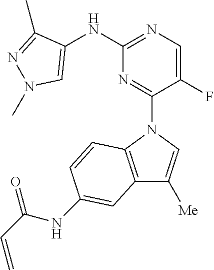 [SMILES: C=CC(=O)Nc1ccc2c(c1)c(C)cn2-c1nc(Nc2cn(C)nc2C)ncc1F]

Step 1. N-(1,3-dimethylpyrazol-4-yl)-5-fluoro-4-(3-methyl-5-nitro-indol-1-yl)pyrimidin-2-amine. A suspension of 1-(2-chloro-5-fluoro-pyrimidin-4-yl)-3-methyl-5-nitro-indole (5.50 g, 17.9 mmol), 1,3-dimethylpyrazol-4-amine (2.99 g, 26.9 mmol) and potassium carbonate (6.196 g, 44.8 mmol) in dry 1,4-dioxane (80 mL) was degassed with nitrogen for 10 minutes. Then rac-BINAP (1.675 g, 2.69 mmol) and palladium (II) acetate (403 mg, 1.79 mmol) were added and the resulting mixture was heated at 100° C. for 90 min. After cooling to room temperature, the reaction mixture was filtered. The filtrate was evaporated and the residue purified by Interchim 520+, (40 g column, eluent 0-100% DCM:MeOH=9:1/DCM) to afford 6.9 g (99%). LCMS m/z=382 (M+1).

Step 2. 1-[2-[(1,3-dimethylpyrazol-4-yl)amino]-5-fluoro-pyrimidin-4-yl]-3-methyl-indol-5-amine. N-(1,3-dimethylpyrazol-4-yl)-5-fluoro-4-(3-methyl-5-nitro-indol-1-yl)pyrimidin-2-amine (2.0 g, 5.24 mmol), iron (2.343 g, 42.0 mmol) and ammonium chloride (561 mg, 10.5 mmol) were heated in EtOH (107 mL) and water (36 mL) at reflux for 2 h. After cooling to room temperature, DCM (50 mL) was added and the mixture was filtered. The filtrate was diluted with saturated NaHCO₃ solution (50 mL). The layers were separated and the water layer extracted with DCM (2×100 mL). the organic phase was dried over Na₂SO₄ and evaporated to afford 1.7 g (92%), which was used directly in the next step. LCMS m/z=352 (M+1).

Step 3. N-[1-[2-[(1,3-Dimethylpyrazol-4-yl)amino]-5-fluoro-pyrimidin-4-yl]-3-methyl-indol-5-yl]prop-2-enamide. To a solution of 1-[2-[(1,3-dimethylpyrazol-4-yl)amino]-5-fluoro-pyrimidin-4-yl]-3-methyl-indol-5-amine (1.70 g, 4.84 mmol) in DMF (30 mL), was added acryloyl chloride (0.398 mL, 4.84 mmol) and N,N-diisopropylethylamine (2.53 mL, 14.5 mmol). The resulting mixture was stirred at rt for 1 h and then concentrated. The residue was purified by silica gel chromatography (0-50% DCM:MeOH:NH₄OH=90:5:1.5/DCM) to afford 875 mg (45%) as a white solid. LCMS m/z=406 (M+1); ¹H NMR (500 MHz, DMSO-d6) δ: 9.91 (s, 1H), 8.52 (s, 1H), 8.43 (d, J=4.6 Hz, 1H), 8.18 (d, J=9.2 Hz, 1H), 8.00 (s, 1H), 7.71 (s, 1H), 7.64 (s, 1H), 7.41 (d, J=9.2 Hz, 1H), 6.47 (dd, J=17.1, 10.1 Hz, 1H), 6.27 (d, J=17.1 Hz, 1H), 5.73 (d, J=10.4 Hz, 1H), 3.78 (s, 3H), 2.29 (s, 3H), 2.10 (s, 3H).

### Example 181: N-[1-[5-fluoro-2-[[1-(2-hydroxy-2-methyl-propyl)pyrazol-4-yl]amino]pyrimidin-4-yl]-3-methyl-indol-5-yl]prop-2-enamide

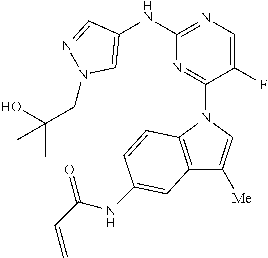 [SMILES: C=CC(=O)Nc1ccc2c(c1)c(C)cn2-c1nc(Nc2cnn(CC(C)(C)O)c2)ncc1F]

Step 1. 2-Methyl-1-(4-nitropyrazol-1-yl)propan-2-ol. A suspension of 4-nitro-1H-pyrazole (7.0 g, 62 mmol, 1 eq), 1-chloro-2-methyl-propan-2-ol (7.6 mL, 74 mmol, 1.2 eq) and potassium carbonate (17.1 g, 124 mmol, 2 eq) in i-PrOH was heated at 80° C. for 6 h. After cooling to room temperature, the reaction mixture was concentrated to a third volume. Water was added and the mixture extracted with EtOAc. The combined organic layers were dried over Na₂SO₄ and concentrated to give a yellow viscous oil (11 g, 97%). LCMS m/z=185.96 (M+1); ¹H NMR (500 MHz, DMSO-d6) δ: 8.65 (s, 1H), 8.25 (s, 1H), 4.83 (s, 1H), 4.09 (s, 2H), 1.09 (s, 6H).

Step 2. 1-(4-Aminopyrazol-1-yl)-2-methyl-propan-2-ol. A solution of 2-methyl-1-(4-nitropyrazol-1-yl)propan-2-ol (16.3 g, 87.8 mmol) in MeOH (200 mL) was added Pd/C (10%, 1.87 g, 1.76 mmol) and the reaction mixture was shaken in a Parr apparatus under 3 bar of H₂ atmosphere at rt for 2 h. The reaction mixture was filtered through a pad of celite to remove the catalyst. The celite pad was washed with MeOH, and the combined solvent was concentrated to give a violet-colored oil (13.8 g, 96%). LCMS m/z=155.96 (M+1); ¹H NMR (500 MHz, DMSO-d6) δ: 7.02 (s, 1H), 6.89 (s, 1H), 4.60 (s, 1H), 3.76-3.85 (m, 4H), 1.00 (s, 6H).

Step 3. 1-(2-Chloro-5-fluoro-pyrimidin-4-yl)-3-methyl-5-nitro-indole. The general procedure described previously was followed. A mixture of 2,4-dichloro-5-fluoro-pyrimidine (1.05 eq), 3-methyl-5-nitro-1H-indole (1 eq), and K₂CO₃ (1.5 eq) in DMF was stirred at rt for 4 h. Reaction mixture was diluted with water, the precipitate that formed was collected and washed with water and diethyl ether, and then dried under vacuum over night to give a yellow solid. LCMS m/z=307 (M+1); ¹H NMR (DMSO-d6, 500 MHz) δ: 9.03 (d, J=3.7 Hz, 1H), 8.57 (d, J=2.1 Hz, 1H), 8.41 (d, J=9.2 Hz, 1H), 8.28 (dd, J=9.2, 2.1 Hz, 1H), 7.91 (s, 1H), 2.40 (s, 3H).

Step 4. 1-[4-[[5-Fluoro-4-(3-methyl-5-nitro-indol-1-yl)pyrimidin-2-yl]amino]pyrazol-1-yl]-2-methyl-propan-2-ol. The following general procedure was used. A suspension of 1-(2-chloro-5-fluoro-pyrimidin-4-yl)-3-methyl-5-nitro-indole (1 eq), 1-(4-aminopyrazol-1-yl)-2-methyl-propan-2-ol (1.2 eq) and 4-methylbenzenesulfonic acid (1 eq) in 1,4-dioxane was heated at reflux (105° C.) for 48 h. The product precipitates during the reaction. The mixture was cooled to rt, diluted with water, neutralized with saturated NaHCO₃ solution and stirred at rt for 30 min. The precipitate was collected, washed thoroughly with water and ethanol then dried under vacuum over night to give a yellow solid. Yield 86%; LCMS m/z=426.08 (M+1); ¹H NMR (DMSO-d6, 500 MHz) δ: 9.44 (s, 1H), 8.63 (d, J=3.7 Hz, 1H), 8.54 (d, J=2.1 Hz, 1H), 8.37 (br d, J=8.9 Hz, 1H), 8.12 (dd, J=9.0, 2.0 Hz, 1H), 7.87 (s, 1H), 7.83 (s, 1H), 7.56 (s, 1H), 4.38 (br s, 1H), 3.99 (s, 2H), 2.42 (s, 3H), 1.10 (s, 6H).

Step 5. 1-[4-[[4-(5-Amino-3-methyl-indol-1-yl)-5-fluoro-pyrimidin-2-yl]amino]pyrazol-1-yl]-2-methyl-propan-2-ol. A suspension of 1-[4-[[5-fluoro-4-(3-methyl-5-nitro-indol-1-yl)pyrimidin-2-yl]amino]pyrazol-1-yl]-2-methyl-propan-2-ol (1 eq), Pd/C (10.0%, 0.1 eq) suspended in THF was stirred in an autoclave under a H₂ atmosphere (6 bar at rt) overnight. The reaction mixture was filtered through a pad of celite and the filter cake was washed with MeOH. The solvent was concentrated, the solid suspended in MeOH, filtered and dried in the vacuum oven to give a beige solid. Yield 71%; LCMS m/z=396.29 (M+H); ¹H NMR (500 MHz, DMSO-d6) δ: 9.15 (s, 1H), 8.40 (d, J=4.9 Hz, 1H), 8.12 (br d, J=8.5 Hz, 1H), 7.86 (s, 1H), 7.55 (s, 1H), 7.49 (s, 1H), 6.73 (d, J=1.8 Hz, 1H), 6.64 (dd, J=8.7, 2.0 Hz, 1H), 4.68 (s, 2H), 4.41 (s, 1H), 3.98 (s, 2H), 2.21 (s, 3H), 1.10 (s, 6H).

Step 6. N-[1-[5-Fluoro-2-[[1-(2-hydroxy-2-methyl-propyl)pyrazol-4-yl]amino]pyrimidin-4-yl]-3-methyl-indol-5-yl]prop-2-enamide. To a solution of 1-[4-[[4-(5-amino-3-methyl-indol-1-yl)-5-fluoro-pyrimidin-2-yl]amino]pyrazol-1-yl]-2-methyl-propan-2-ol (1 eq) and N,N-diisopropylethylamine (2.5 eq) in dry THF, was added acryloyl chloride (1 eq) in 4 portions at 15 min intervals. The mixture was then stirred at rt for 2 h. Water was added and the mixture was concentrated. The residue was diluted with ethanol, water was slowly added and stirred for 30 min, upon which time the product precipitated as a yellow slurry. The solvent was decanted, and the product was collected by filtration and dried under vacuum to give a pale-yellow solid. Yield 85%; LCMS m/z=450 (M+H); ¹H NMR (500 MHz, DMSO-d6) δ: 9.94 (s, 1H), 9.28 (s, 1H), 8.50 (d, J=4.6 Hz, 1H), 8.31 (d, J=8.2 Hz, 1H), 8.01 (s, 1H), 7.88 (s, 1H), 7.64 (s, 1H), 7.57 (s, 1H), 7.49 (d, J=8.5 Hz, 1H), 6.46-6.44 (m, 1H), 6.32-6.24 (m, 1H), 5.76-5.70 (m, 1H), 4.41 (s, 1H), 3.99 (s, 2H), 2.30 (s, 3H), 1.10 (s, 6H).

### Example 185. N-[1-[5-Fluoro-2-[[1-[(2R)-2-hydroxypropyl]pyrazol-4-yl]amino]pyrimidin-4-yl]-3-methyl-indol-5-yl]prop-2-enamide

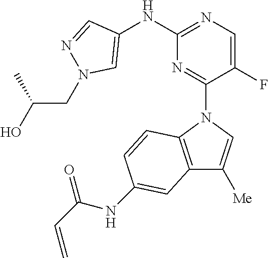 [SMILES: C=CC(=O)Nc1ccc2c(c1)c(C)cn2-c1nc(Nc2cnn(C[C@@H](C)O)c2)ncc1F]

Step 1. (2R)-1-(4-Nitropyrazol-1-yl)propan-2-ol. A suspension of 4-nitro-1H-pyrazole (2.0 g, 17.7 mmol), (2R)-2-methyloxirane (1.86 mL, 26.5 mmol) and cesium carbonate (11500 mg, 35.4 mmol) in dry DMF (25 mL) was heated at 100° C. for 30 min. Upon complete conversion, the reaction mixture was diluted with water and extracted with EtOAc. The combined organic extracts were washed with a 5% aqueous LiCl solution, dried over Na₂SO₄ and concentrated to dryness. The residue was purified by silica gel chromatography (cyclohexane:EtOAc=2:1) to give a as yellow oil (1.7 g, 38%). LCMS m/z=172 (M+1).

Step 2. (2R)-1-(4-Aminopyrazol-1-yl)propan-2-ol. To a solution of (2R)-1-(4-nitropyrazol-1-yl)propan-2-ol (674 mg, 3.94 mmol) in methanol (70 mL) was added Pd/C (10.0%, 169 mg, 0.158 mmol) and the reaction mixture was stirred under an atmosphere of hydrogen (2 bar) for 3 h at RT. The reaction mixture was filtered over celite and rinsed thoroughly with MeOH. The filtrate was concentrated to dryness to give a violet oil (553 mg, 99%). LCMS m/z=142 (M+1); ¹H NMR (300 MHz, DMSO-d6) δ: 6.99 (s, 1H), 6.87 (s, 1H), 4.79 (d, J=4.7 Hz, 1H), 3.68-3.96 (m, 5H), 0.95 (d, J=6.1 Hz, 3H).

Step 3. (2R)-1-[4-[[5-Fluoro-4-(3-methyl-5-nitro-indol-1-yl)pyrimidin-2-yl]amino]pyrazol-1-yl]propan-2-ol. A suspension of 1-(2-chloro-5-fluoro-pyrimidin-4-yl)-3-methyl-5-nitro-indole (400 mg, 1.30 mmol), (2R)-1-(4-aminopyrazol-1-yl)propan-2-ol (203 mg, 1.43 mmol) and potassium carbonate (361 mg, 2.61 mmol) in dry 1,4-dioxane (15 mL) was degassed with Argon for 10 minutes. Then Xantphos (113 mg, 0.196 mmol) and palladium (II) acetate (29.3 mg, 0.130 mmol) were added and the reaction mixture was irradiated in MW at 80° C. for 45 min. After cooling to RT, the reaction mixture was concentrated, the residue was dissolved in DCM, washed with saturated NaHCO₃ solution and brine, dried over Na₂SO₄ and concentrated. The crude product was triturated with MeOH, collected and dried to give a yellowish solid (428 mg, 80%). LCMS m/z=412 (M+1).

Step 4. (2R)-1-[4-[[4-(5-Amino-3-methyl-indol-1-yl)-5-fluoro-pyrimidin-2-yl]amino]pyrazol-1-yl]propan-2-ol. A suspension of (2R)-1-[4-[[5-fluoro-4-(3-methyl-5-nitro-indol-1-yl)pyrimidin-2-yl]amino]pyrazol-1-yl]propan-2-ol (92.0%, 428 mg, 0.957 mmol), iron (641 mg, 11.5 mmol) and ammonium chloride (205 mg, 3.83 mmol) in EtOH (15 mL) and water (3 mL) was stirred at reflux for 1 hour. Upon complete conversion, reaction mixture was diluted with DCM, filtered and the solvent was concentrated in vacuo. The residue was dissolved in DCM (100 mL), washed with saturated NaHCO₃ solution and brine, dried over Na₂SO₄ and concentrated to dryness to give a yellow solid (335 mg, 92%). LCMS m/z=382 (M+1)

Step 5. Preparation of N-[1-[5-fluoro-2-[[1-[(2R)-2-hydroxypropyl]pyrazol-4-yl]amino]pyrimidin-4-yl]-3-methyl-indol-5-yl]prop-2-enamide. To a solution of (2R)-1-[4-[[4-(5-amino-3-methyl-indol-1-yl)-5-fluoro-pyrimidin-2-yl]amino]pyrazol-1-yl]propan-2-ol (335 mg, 0.826 mmol) in dry THF (10 ml) was added DIPEA (0.225 mL, 1.65 mmol) followed by acryloyl chloride (0.0671 mL, 0.826 mmol). The resulting mixture was stirred at RT for 30 min. Water was added and the reaction mixture was concentrated, the residue was dissolved in DCM (100 mL), washed with saturated NaHCO₃ solution and brine, dried over Na₂SO₄ and concentrated. The product was purified by silica gel chromatography (DCM:MeOH=10:1) to give a white solid (155 mg, 43%). LCMS m/z=436 (M+1); ¹H NMR (500 MHz, DMSO-d6) δ: 9.93 (s, 1H), 9.27 (s, 1H), 8.49 (d, J=4.6 Hz, 1H), 8.31 (d, J=8.9 Hz, 1H), 8.01 (d, J=1.5 Hz, 1H), 7.86 (s, 1H), 7.64 (s, 1H), 7.55 (s, 1H), 7.50 (dd, J=8.9, 1.8 Hz, 1H), 6.48 (dd, J=17.1, 10.4 Hz, 1H), 6.28 (dd, J=16.9, 2.0 Hz, 1H), 5.73 (dd, J=10.1, 1.8 Hz, 1H), 4.60 (d, J=4.0 Hz, 1H), 3.94-4.03 (m, 3H), 2.30 (d, J=0.9 Hz, 3H), 1.07 (d, J=5.8 Hz, 3H).

### Example 186. N-[1-[5-Fluoro-2-[[1-[(2S)-2-hydroxypropyl]pyrazol-4-yl]amino]pyrimidin-4-yl]-3-methyl-indol-5-yl]prop-2-enamide

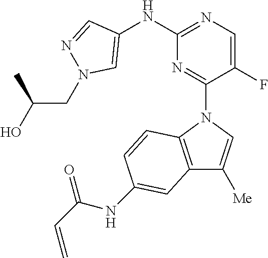 [SMILES: C=CC(=O)Nc1ccc2c(c1)c(C)cn2-c1nc(Nc2cnn(C[C@H](C)O)c2)ncc1F]

Step 1. (2S)-1-(4-Aminopyrazol-1-yl)propan-2-ol was synthesized using the procedure for (2R)-1-(4-aminopyrazol-1-yl)propan-2-ol Example 185, starting with 4-nitro-1H-pyrazole and (2S)-2-methyloxirane, to give an oil. LCMS m/z=142 (M+1), ¹H NMR (300 MHz, DMSO-d6) δ: 6.99 (s, 1H), 6.87 (s, 1H), 4.79 (d, J=4.7 Hz, 1H), 3.71-3.84 (m, 5H), 0.95 (d, J=5.9 Hz, 3H).

Step 2. N-[1-[5-Fluoro-2-[[1-[(2S)-2-hydroxypropyl]pyrazol-4-yl]amino]pyrimidin-4-yl]-3-methyl-indol-5-yl]prop-2-enamide. This example was synthesized using the procedure for Example 185, with 1-(2-chloro-5-fluoro-pyrimidin-4-yl)-3-methyl-5-nitro-indole and (2S)-1-(4-aminopyrazol-1-yl)propan-2-ol, to give a white solid. LCMS mlz=436 (M+1); ¹H NMR (500 MHz, DMSO-d6, 353 K) δ: 9.94 (s, 1H), 9.27 (s, 1H), 8.50 (d, J=4.9 Hz, 1H), 8.31 (d, J=8.9 Hz, 1H), 8.01 (s, 1H), 7.86 (s, 1H), 7.64 (s, 1H), 7.55 (s, 1H), 7.49 (dd, J=8.9, 1.8 Hz, 1H), 6.48 (dd, J=16.9, 10.2 Hz, 1H), 6.28 (dd, J=17.1, 1.8 Hz, 1H), 5.73 (dd, J=10.4, 1.8 Hz, 1H), 4.60 (d, J=3.1 Hz, 1H), 3.94-4.03 (m, 3H), 2.30 (s, 3H), 1.07 (d, J=5.5 Hz, 3H).

The Examples in Table 1 below, synthesized using general methods described above, are embodiments of the invention.

TABLE 1

| | | |
|---|---|---|
| Example 1 N-[1-[5-chloro-2-[4-(4- methylpiperazin-1- yl)anilino]pyrimidin-4-yl]indol-5- yl]prop-2-enamide | 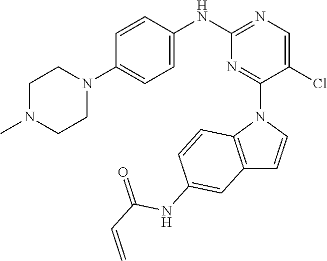 [SMILES: C=CC(=O)Nc1ccc2c(ccn2-c2nc(Nc3ccc(N4CCN(C)CC4)cc3)ncc2Cl)c1] | LCMS m/z = 488, 490 (M + 1, 1 Cl isotopic distribution); ¹H NMR (500 MHz, DMSO- d6) δ: 2.21 (s, 3H) 2.41 - 2.46 (m, 4H) 3.02 - 3.09 (m, 4H) 5.75 (dd, J = 10.2, 1.9 Hz, 1H) 6.26 (dd, J = 16.9, 1.9 Hz, 1H) 6.47 (dd, J = 16.7, 10.1 Hz, 1H) 6.76 (d, J = 3.36 Hz, 1H) 6.87 (br d, J = 8.8 Hz, 2H) 7.40 (br d, J = 8.8 Hz, 1H) 7.53 (d, J = 9.1 Hz, 2H) 7.80 (br d, J = 7.9 Hz, 1H) 7.85 (d, J = 3.6 Hz, 1H) 8.13 (d, J = 1.5 Hz, 1H) 8.64 (s, 1H) 9.77 (s, 1H) 10.14 (s, 1H) |
| Example 2 N-[1-[5-chloro-2-[(1-methylpyrazol- 4-yl)amino]pyrimidin-4-yl]indol-5- yl]prop-2-enamide | 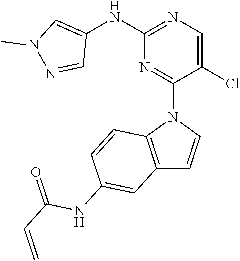 [SMILES: C=CC(=O)Nc1ccc2c(ccn2-c2nc(Nc3cnn(C)c3)ncc2Cl)c1] | LCMS m/z = 394, 396 (M + 1, 1 Cl isotopic distribution); ¹H NMR (600 MHz, DMSO-d6, 353 K) δ: 9.85 (br s, 1H), 9.57 (br s, 1H), 8.57 (s, 1H), 8.04 (s, 1H), 7.82 (d, J = 3.5 Hz, 1H), 7.79 - 7.75 (m, 2H), 7.51 - 7.50 (m, 1H), 7.43 (dd, J = 1.9, 8.9 Hz, 1H), 6.73 (d, J = 3.3 Hz, 1H), 6.48 - 6.43 (m, 1H), 6.25 (dd, J = 1.8, 17.1 Hz, 1H), 5.70 (dd, J = 1.9, 10.2 Hz, 1H), 3.77 (s, 3H) |
| Example 3 N-[1-[5-chloro-2-[4-[2- (dimethylamino)ethyl-methyl- amino]anilino]pyrimidin-4-yl]indol-5- yl]prop-2-enamide | 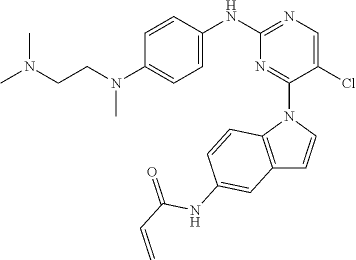 [SMILES: C=CC(=O)Nc1ccc2c(ccn2-c2nc(Nc3ccc(N(C)CCN(C)C)cc3)ncc2Cl)c1] | LCMS m/z = 490, 492 (M + 1, 1 Cl isotopic distribution); ¹H NMR (500 MHz, DMSO-d6) δ: 10.13 (s, 1 H), 9.64 (s, 1 H), 8.60 (s, 1 H), 8.09 (d, J = 1.83 Hz, 1 H), 7.85 (d, J = 3.36 Hz, 1 H), 7.80 (bs, 1H), 7.44 (d, J = 9.16 Hz, 2 H), 7.40 (br d, J = 8.85 Hz, 1 H), 6.75 (d, J = 3.36 Hz, 1 H), 6.63 (br d, J = 8.85 Hz, 2 H), 6.47 (dd, J = 17.09 Hz, 10.07 Hz, 1H), 6.26 (dd, J = 16.97 Hz, 1.98 Hz, 1 H), 5.74 (dd, J = 10.07 Hz, 2.14 Hz, 1 H), 3.36 - 3.44 (m, 2 H), 2.86 (s, 3 H), 2.33 - 2.37 (m, 2H), 2.16 (s, 6 H) |
| Example 4 N-[1-[5-chloro-2-[4-(4- methylpiperazin-1- yl)anilino]pyrimidin-4-yl]indol-5- yl]propanamide | 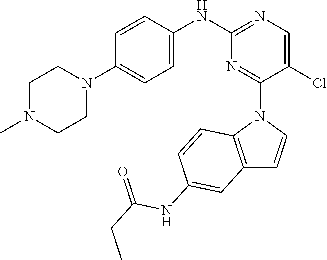 [SMILES: CCC(=O)Nc1ccc2c(ccn2-c2nc(Nc3ccc(N4CCN(C)CC4)cc3)ncc2Cl)c1] | LCMS m/z = 490, 492 (M + 1, 1 Cl isotopic distribution); ¹H NMR (500 MHz, DMSO- d6) δ: 9.83 (s, 1H), 9.75 (br.s, 1H), 8.62 (s, 1H), 8.03 (d, J = 1.7 Hz, 1H), 7.83 (d, J = 3.5 Hz, 1H), 7.73-7.80 (m, 1H), 7.52 (d, J = 8.9 Hz, 2H), 7.31 (d, J = 8.7 Hz, 1H), 6.86 (d, J = 8.9 Hz, 2H), 6.73 (d, J = 3.5 Hz, 1H), 3.01-3.10 (m, 4H), 2.41-2.46 (m, 4H), 2.33 (q, J = 7.5 Hz, 2H), 2.20 (s, 3H), 1.10 (t, J = 7.5 Hz, 3H) |
| Example 5 N-[4-methyl-1-[5-methyl-2-[3-(2- morpholinoethoxy)anilino]pyrimidin- 4-yl]indol-5-yl]prop-2-enamide | 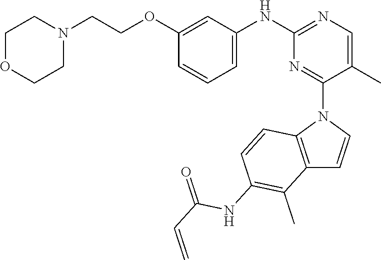 [SMILES: C=CC(=O)Nc1ccc2c(ccn2-c2nc(Nc3cccc(OCCN4CCOCC4)c3)ncc2C)c1C] | LCMS m/z = 513 (M + 1); ¹H NMR (500 MHz, DMSO-d6) δ: 9.69 (s, 1H), 9.62 (s, 1H), 8.56 (s, 1H), 7.76 (d, J = 3.4 Hz, 1H), 7.53-7.56 (m, J = 6.9 Hz, 2H), 7.26 (d, J = 7.9 Hz, 1H), 7.20 (d, J = 8.9 Hz, 1H), 7.11 (t, J = 8.2 Hz, 1H), 6.82 (d, J = 3.4 Hz, 1H), 6.49-6.57 (m, 2H), 6.24 (dd, J = 17.1, 1.8 Hz, 1H), 5.73 (dd, J = 10.1, 2.1 Hz, 1H), 3.94 (t, J = 5.8 Hz, 2H), 3.54 (t, J = 4.9 Hz, 4H), 2.60 (t, J = 5.8 Hz, 2H), 2.38 (br s, 7H), 2.20 (s, 3H) |
| Example 6 N-[1-[5-chloro-2-[3-(2- morpholinoethoxy)anilino]pyrimidin- 4-yl]indol-5-yl]prop-2-enamide | 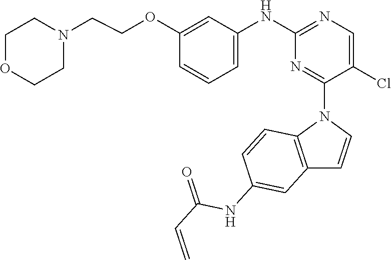 [SMILES: C=CC(=O)Nc1ccc2c(ccn2-c2nc(Nc3cccc(OCCN4CCOCC4)c3)ncc2Cl)c1] | LCMS m/z = 519, 521 (M + 1, 1 Cl isotopic distribution); ¹H NMR (500 MHz, DMSO-d6) δ: 10.16 (s, 1H), 10.02 (s, 1H), 8.73 (s, 1H), 8.12 (d, J = 1.8 Hz, 1H), 7.88 (d, J = 3.7 Hz, 1H), 7.83 (d, J = 8.9 Hz, 1H), 7.51 (s, 1H), 7.43 (dd, J = 7.9, 2.1 Hz, 1H), 7.22 (d, J = 8.5 Hz, 1H), 7.16 (t, J = 8.2 Hz, 1H), 6.78 (d, J = 3.7 Hz, 1H), 6.57 (dd, J = 7.3, 2.1 Hz, 1H), 6.44-6.51 (m, 1H), 6.27 (dd, J = 15.9, 1.8 Hz, 1H), 5.75 (dd, J = 10.2, 2.0 Hz, 1H), 3.91 (t, J = 5.3 Hz, 2H), 3.52 (t, J = 4.6 Hz, 4H), 2.59 (t, J = 5.8 Hz, 2H), 2.36 (br s, 4H) |
| Example 7 N-[1-[5-chloro-2-[3-(2- morpholinoethoxy)anilino]pyrimidin- 4-yl]-4-methyl-indol-5-yl]prop-2- enamide | 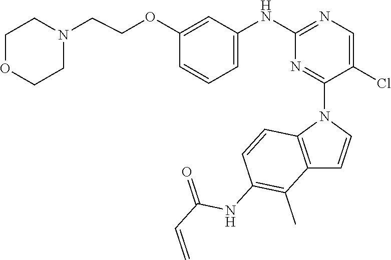 [SMILES: C=CC(=O)Nc1ccc2c(ccn2-c2nc(Nc3cccc(OCCN4CCOCC4)c3)ncc2Cl)c1C] | LCMS m/z = 533, 535 (M + 1, 1 Cl isotopic distribution); ¹H NMR (DMSO-d6, 500 MHz) δ: 10.03 (s, 1H), 9.64 (s, 1H), 8.75 (s, 1H), 7.85 (d, J = 3.54 Hz, 1H), 7.63 (d, J = 8.73 Hz, 1H), 7.49-7.47 (m, 1H), 7.26-7.21 (m, 2H), 7.15 (t, J = 8.10 Hz, 1H), 6.86 (d, J = 3.46 Hz, 1H), 6.59-6.49 (m, 2H), 6.24 (dd, J = 2.0, 17.31 Hz, 1H), 5.74 (dd, J = 1.85, 10.20 Hz, 1H), 3.98-3.94 (m, 2H), 3.56-3.51 (m, 4H), 2.64-2.59 (m, 2H), 2.42-2.36 (m, 7H) |
| Example 8 N-[3-methyl-1-[5-methyl-2-[(1- methylpyrazol-4-yl)amino]pyrimidin- 4-yl]indol-5-yl]prop-2-enamide | 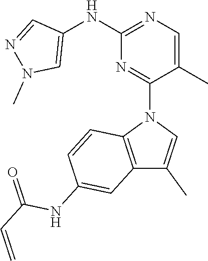 [SMILES: C=CC(=O)Nc1ccc2c(c1)c(C)cn2-c1nc(Nc2cnn(C)c2)ncc1C] | LCMS m/z = 388 (M + 1); ¹H NMR (DMSO-d6, 500 MHz) δ: 10.14 (s, 1H), 9.45 (br s, 1H), 8.42 (s, 1H), 8.06 (d, J = 1.5 Hz, 1H), 7.88 - 7.68 (m, 2H), 7.57 - 7.55 (m, 1H), 7.48 (s, 1H), 7.40 (dd, J = 9.0, 2.0 Hz, 1H), 6.48 (dd, J = 16.9, 10.2 Hz, 1H), 6.26 (dd, J = 16.9, 2.0 Hz, 1H), 5.76 - 5.72 (m, 1H), 3.75 (br s, 3H), 2.29 (s, 3H), 2.21 (s, 3H) |
| Example 9 N-[3-methyl-1-[5-methyl-2-[3-(2- morpholinoethoxy)anilino]pyrimidin- 4-yl]indol-5-yl]prop-2-enamide | 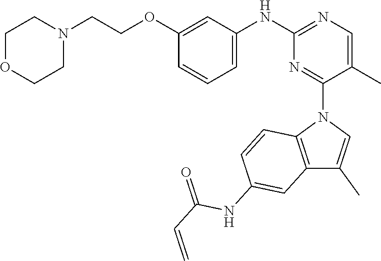 [SMILES: C=CC(=O)Nc1ccc2c(c1)c(C)cn2-c1nc(Nc2cccc(OCCN3CCOCC3)c2)ncc1C] | LCMS m/z = 513 (M + 1); ¹H NMR (DMSO-d6, 300 MHz) δ: 10.13 (s, 1H), 9.63 (s, 1H), 8.49 (s, 1H), 8.05 (d, J = 1.7 Hz, 1H), 7.78 (d, J = 8.9 Hz, 1H), 7.60 - 7.55 (m, 2H), 7.39 (dd, J = 8.9, 2.1 Hz, 1H), 7.29 - 7.16 (m, 1H), 7.14 - 7.03 (m, 1H), 6.53 - 6.42 (m, 2H), 6.29 - 6.17 (m, 1H), 5.77 - 5.69 (m, 1H), 3.92 - 3.85 (m, 2H), 3.55 - 3.48 (m, 4H), 2.60 - 2.54 (m, 2H), 2.39 - 2.31 (m, 4H), 2.29 (d, J = 1.0 Hz, 3H), 2.24 (s, 3H) |
| Example 10 N-[1-[5-chloro-2-[4-(2- morpholinoethoxy)anilino]pyrimidin- 4-yl]indol-5-yl]prop-2-enamide | 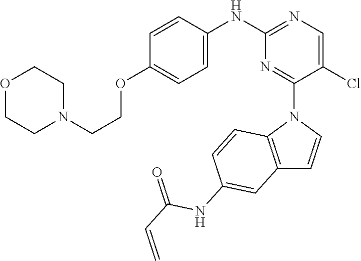 [SMILES: C=CC(=O)Nc1ccc2c(ccn2-c2nc(Nc3ccc(OCCN4CCOCC4)cc3)ncc2Cl)c1] | LCMS m/z = 519, 521 (M + 1, 1 Cl isotopic distribution); ¹H NMR (300 MHz, DMSO-d6) δ: 10.13 (s, 1H), 9.83 (s, 1H), 8.65 (s, 1H), 8.10 (d, J = 1.7 Hz, 1H), 7.85 (d, J = 3.7 Hz, 1H), 7.79 (br d, J = 8.7 Hz, 1H), 7.61 - 7.54 (m, J = 9.1 Hz, 2H), 7.40 (dd, J = 1.7, 9.0 Hz, 1H), 6.91 - 6.83 (m, J = 9.1 Hz, 2H), 6.76 (d, J = 3.1 Hz, 1H), 6.46 (dd, J = 10.1, 16.9 Hz, 1H), 6.25 (dd, J = 2.2, 17.0 Hz, 1H), 5.77 - 5.70 (m, 1H), 4.03 (t, J = 5.8 Hz, 2H), 3.61 - 3.51 (m, 4H), 2.65 (t, J = 6.4 Hz, 2H), 2.47 - 2.43 (m, 4H) |
| Example 11 N-[1-[5-chloro-2-(2-ethoxy-4- morpholino-anilino)pyrimidin-4- yl]indol-5-yl]prop-2-enamide | 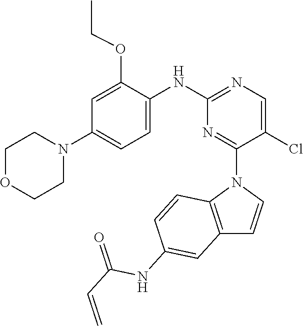 [SMILES: C=CC(=O)Nc1ccc2c(ccn2-c2nc(Nc3ccc(N4CCOCC4)cc3OCC)ncc2Cl)c1] | LCMS m/z = 518, 520 (M + 1, 1 Cl isotopic distribution); ¹H NMR (DMSO-d6, 500 MHz) δ: 10.13 (s, 1H), 8.64 (br s, 1H), 8.57 (s, 1H), 8.05 (s, 1H), 7.85 (d, 1H, J = 3.4 Hz), 7.72 (br d, 1H, J = 8.9 Hz), 7.46 (br d, 1H, J = 8.5 Hz), 7.37 (br d, 1H, J = 8.9 Hz), 6.74 (d, 1H, J = 3.4 Hz), 6.65 (br s, 1H), 6.43- 6.50 (m, 2H), 6.22-6.29 (m, 1H), 5.74 (dd, 1H, J = 1.8, 10.1 Hz), 4.01-4.11 (m, 2H, J = 6.4 Hz), 3.74 (br s, 4H), 3.10 (br s, 4H), 1.26 (t, 3H, J = 7.0 Hz) |
| Example 12 N-[1-[5-chloro-2-[(3-methoxy-1- methyl-pyrazol-4-yl)amino]- pyrimidin-4-yl]indol-5-yl]prop-2- enamide | 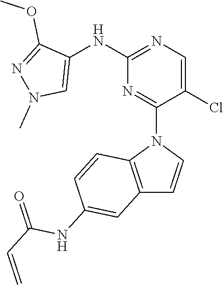 [SMILES: C=CC(=O)Nc1ccc2c(ccn2-c2nc(Nc3cn(C)nc3OC)ncc2Cl)c1] | LCMS m/z = 424, 426 (M + 1 1 Cl isotopic distribution); ¹H NMR (500 MHz, DMSO-d6) δ: 10.13 (s, 1H), 8.92 (s, 1H), 8.55 (s, 1H), 8.09 (s, 1H), 7.87 (d, J = 3.4 Hz, 1H), 7.54-7.83 (m, 2H), 7.36 (br s, 1H), 6.74 (d, J = 3.1 Hz, 1H), 6.46 (dd, J = 17.1, 10.1 Hz, 1H), 6.26 (dd, J = 17.1, 1.8 Hz, 1H), 5.74 (dd, J = 10.2, 1.7 Hz, 1H), 3.82 (s, 3H), 3.64 (s, 3H) |
| Example 13 N-[1-[5-chloro-2-[2-(2- morpholinoethoxy)anilino]pyrimidin- 4-yl]indol-5-yl]prop-2-enamide | 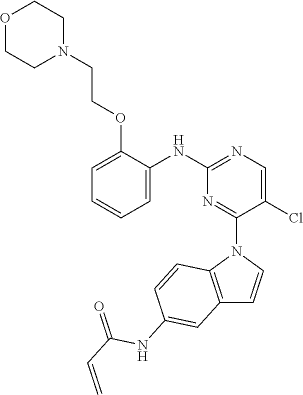 [SMILES: C=CC(=O)Nc1ccc2c(ccn2-c2nc(Nc3ccccc3OCCN3CCOCC3)ncc2Cl)c1] | LCMS m/z = 519, 521 (M + 1, 1 Cl isotopic distribution); ¹H NMR (500 MHz, DMSO-d6) δ: 10.13 (s, 1H), 9.01 (s, 1H), 8.66 (s, 1H), 8.09 (d, J = 1.8 Hz, 1H), 7.87 (d, J = 3.4 Hz, 1H), 7.76 (d, J = 7.9 Hz, 1H), 7.71 (d, J = 8.9 Hz, 1H), 7.32 (d, J = 8.9 Hz, 1H), 7.10-7.15 (m, 2H), 6.94 (t, J = 7.2 Hz, 1H), 6.76 (d, J = 3.4 Hz, 1H), 6.46 (dd, J = 16.9, 10.2 Hz, 1H), 6.26 (dd, J = 16.9, 2.0 Hz, 1H), 5.74 (dd, J = 10.1, 1.8 Hz, 1H), 4.16 (t, J = 5.3 Hz, 2H), 3.49 (t, J = 4.6 Hz, 4H), 2.60 (t, J = 5.5 Hz, 2H), 2.34-2.40 (m, 4H) |
| Example 14 N-[1-[5-chloro-2-[(3-ethoxy-1- methyl-pyrazol-4-yl)amino]pyrimidin- 4-yl]indol-5-yl]prop-2-enamide | 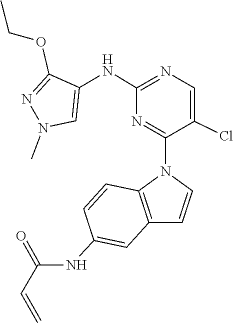 [SMILES: C=CC(=O)Nc1ccc2c(ccn2-c2nc(Nc3cn(C)nc3OCC)ncc2Cl)c1] | LCMS m/z = 438, 440 (M + 1, 1 Cl isotopic distribution); ¹H NMR (300 MHz, DMSO-d6) δ: 10.13 (s, 1H), 8.87 (s, 1H), 8.54 (s, 1H), 8.09 (s, 1H), 7.86 (d, J = 3.7 Hz, 1H), 7.75 (br s, 1H), 7.60 (s, 1H), 7.35 (br s, 1H), 6.73 (d, J = 3.3 Hz, 1H), 6.48 - 6.40 (m, 1H), 6.30 - 6.21 (m, 1H), 5.76 - 5.70 (m, 1H), 4.16 (q, J = 6.9 Hz, 2H), 3.62 (s, 3H), 1.27 (t, J = 7.0 Hz, 3H) |
| Example 15 N-[1-[5-chloro-2-(4- morpholinoanilino)pyrimidin-4- yl]indol-5-yl]prop-2-enamide | 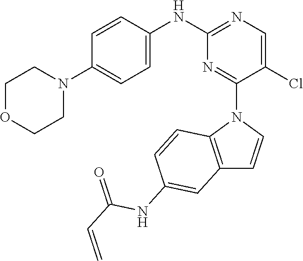 [SMILES: C=CC(=O)Nc1ccc2c(ccn2-c2nc(Nc3ccc(N4CCOCC4)cc3)ncc2Cl)c1] | LCMS m/z = 475, 477 (M + 1, 1 Cl isotopic distribution); ¹H NMR (500 MHz, DMSO-d6) δ: 10.14 (s, 1H), 9.79 (s, 1H), 8.64 (s, 1H), 8.12 (s, 1H), 7.86 (d, J = 4.6 Hz, 1H), 7.79 (br s, 1H), 7.56 (d, J = 8.9 Hz, 2H), 7.40 (d, J = 8.9 Hz, 1H), 6.88 (d, J = 8.9 Hz, 2H), 6.77 (d, J = 3.4 Hz, 1H), 6.47 (dd, J = 16.9, 10.2 Hz, 1H), 6.26 (dd, J = 16.8, 2.1 Hz, 1H), 5.75 (dd, J = 10.1, 1.8 Hz, 1H), 3.70- 3.74 (m, 4H), 3.01-3.05 (m, 4H) |
| Example 16 N-[1-[5-chloro-2-(3-fluoro-4- morpholino-anilino)pyrimidin-4- yl]indol-5-yl]prop-2-enamide | 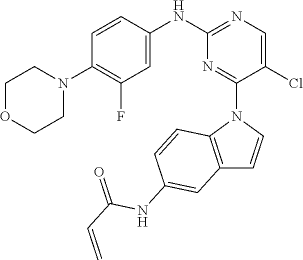 [SMILES: C=CC(=O)Nc1ccc2c(ccn2-c2nc(Nc3ccc(N4CCOCC4)c(F)c3)ncc2Cl)c1] | LCMS m/z = 493, 495 (M + 1, 1 Cl isotopic distribution); ¹H NMR (DMSO-d6, 500 MHz) δ: 10.16 (s, 1H), 10.06 (s, 1H), 8.72 (s, 1H), 8.14 (s, 1H), 7.87 (d, J = 3.51 Hz, 1H), 7.83 (d, J = 8.89 Hz, 1H), 7.70 (dd, J = 2.07, 15.27 Hz, 1H), 7.42 (d, J = 8.89 Hz, 1H), 7.37 (d, J = 8.74 Hz, 1H), 6.98 (t, J = 9.24 Hz, 1H), 6.78 (d, J = 3.41 Hz, 1H), 6.48 (dd, J = 10.13, 16.85 Hz, 1H), 6.27 (dd, J = 1.63, 16.94 Hz, 1H), 5.75 (dd, J = 1.78, 10.03 Hz, 1H), 3.75- 3.70 (m, 4H), 2.96-2.92 (m, 4H) |
| Example 17 N-[1-[5-chloro-2-(3-fluoro-4- morpholino-anilino)pyrimidin-4-yl]-4- methyl-indol-5-yl]prop-2-enamide | 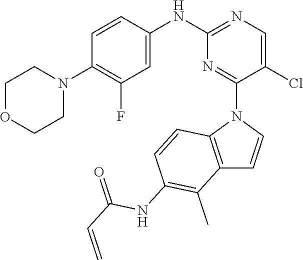 [SMILES: C=CC(=O)Nc1ccc2c(ccn2-c2nc(Nc3ccc(N4CCOCC4)c(F)c3)ncc2Cl)c1C] | LCMS m/z = 507, 509 (M + 1, 1 Cl isotopic distribution); ¹H NMR (500 MHz, DMSO- d6) δ: 2.37 (s, 3H) 2.89 - 2.95 (m, 4H) 3.67 - 3.74 (m, 4H) 5.73 (dd, J = 10.1, 2.0 Hz, 1H) 6.26 (d, J = 2.0 Hz, 1H) 6.47 - 6.58 (m, 1H) 6.86 (d, J = 2.9 Hz, 1H) 6.96 (t, J = 9.3 Hz, 1H) 7.19 - 7.24 (m, 1H) 7.36 (dd, J = 8.7, 1.7 Hz, 1H) 7.68 (dd, J = 15.2, 2.5 Hz, 2H) 7.84 (d, J = 3.6 Hz, 1H) 8.73 (s, 1H) 9.65 (s, 1H) 10.08 (s, 1H) |
| Example 18 N-[1-[5-chloro-2-[(1-methylpyrazol- 4-yl)amino]pyrimidin-4-yl]-4-methyl- indol-5-yl]prop-2-enamide | 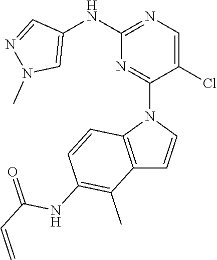 [SMILES: C=CC(=O)Nc1ccc2c(ccn2-c2nc(Nc3cnn(C)c3)ncc2Cl)c1C] | LCMS m/z = 408, 410 (M + 1, 1 Cl isotopic distribution); ¹H NMR (DMSO-d6, 500 MHz, 353K) δ: 9.64 (br s, 1H), 9.39 (br s, 1H), 8.62 - 8.58 (m, 1H), 7.83 - 7.78 (m, 2H), 7.60 (br d, J = 8.5 Hz, 1H), 7.51 (s, 1H), 7.25 (br d, J = 8.5 Hz, 1H), 6.84 (d, J = 3.4 Hz, 1H), 6.58 - 6.44 (m, 1H), 6.25 (dd, J = 17.2, 2.0 Hz, 1H), 5.71 (dd, J = 10.4, 1.5 Hz, 1H), 3.78 (s, 3H), 2.40 (s, 3H) |
| Example 19 N-[3-methyl-1-[5-methyl-2-[4-(2- morpholinoethoxy)anilino]pyrimidin- 4-yl]indol-5-yl]prop-2-enamide | 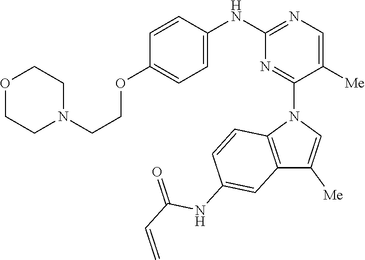 [SMILES: C=CC(=O)Nc1ccc2c(c1)c(C)cn2-c1nc(Nc2ccc(OCCN3CCOCC3)cc2)ncc1C] | LCMS m/z = 513.17 (M + 1); ¹H NMR (500 MHz, DMSO-d6) δ: 10.14 (s, 1H), 9.43 (s, 1H), 8.43 (s, 1H), 8.05 (d, J = 1.5 Hz, 1H), 7.76 (d, J = 8.9 Hz, 1H), 7.62 (d, J = 9.2 Hz, 2H), 7.56 (s, 1H), 7.38 (dd, J = 8.9, 1.8 Hz, 1H), 6.84 (d, J = 8.9 Hz, 2H), 6.51 - 6.44 (m, 1H), 6.29 - 6.23 (m, 1H), 5.74 (dd, J = 10.1, 1.8 Hz, 1H), 4.05 - 3.99 (m, 2H), 3.57 (t, J = 4.6 Hz, 4H), 2.68 - 2.63 (m, 2H), 2.47 - 2.44 (m, 4H), 2.29 (s, 3H), 2.21 (s, 3H) |
| Example 20 N-[1-[5-chloro-2-(2- ethoxyanilino)pyrimidin-4-yl]indol-5- yl]prop-2-enamide | 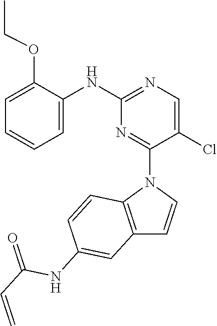 [SMILES: C=CC(=O)Nc1ccc2c(ccn2-c2nc(Nc3ccccc3OCC)ncc2Cl)c1] | LCMS m/z = 434 (M + 1, 1 Cl isotopic distribution); ¹H NMR (500 MHz, DMSO-d6) δ: 10.13 (s, 1H), 8.72 (s, 1H), 8.66 (s, 1H), 8.10 (s, 1H), 7.87 (d, J = 3.4 Hz, 1H), 7.79 (d, J = 7.9 Hz, 1H), 7.74 (d, J = 8.9 Hz, 1H), 7.34 (br d, J = 8.9 Hz, 1H), 7.12 - 7.05 (m, 2H), 6.90 (t, J = 7.5 Hz, 1H), 6.76 (d, J = 3.4 Hz, 1H), 6.46 (dd, J = 16.9, 10.2 Hz, 1H), 6.26 (br d, J = 16.8 Hz, 1H), 5.74 (br d, J = 10.1 Hz, 1H), 4.09 (q, J = 6.8 Hz, 2H), 1.31 (t, J = 6.9 Hz, 3H) |
| Example 21 N-[1-[5-chloro-2-[4-(2- morpholinoethoxy)anilino]pyrimidin- 4-yl]-4-methyl-indol-5-yl]prop-2- enamide | 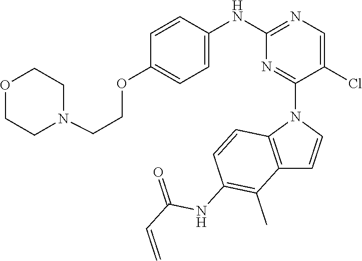 [SMILES: C=CC(=O)Nc1ccc2c(ccn2-c2nc(Nc3ccc(OCCN4CCOCC4)cc3)ncc2Cl)c1C] | LCMS m/z = 535 (M + 1, 1 Cl isotopic distribution); ¹H NMR (300 MHz, DMSO-d6) δ: 9.85 (s, 1H), 9.64 (s, 1H), 8.66 (s, 1H), 7.82 (d, J = 3.5 Hz, 1H), 7.63 - 7.52 (m, 3H), 7.20 (d, J = 8.9 Hz, 1H), 6.90 - 6.80 (m, 3H), 6.52 (dd, J = 10.2, 17.2 Hz, 1H), 6.23 (dd, J = 2.1, 17.1 Hz, 1H), 5.76 - 5.69 (m, 1H), 4.03 (t, J = 5.7 Hz, 2H), 3.59 - 3.52 (m, 4H), 2.64 (t, J = 5.7 Hz, 2H), 2.47 - 2.41 (m, 4H), 2.36 (s, 3H) |
| Example 22 N-[1-[5-chloro-2-(3-fluoro-4- morpholino-anilino)pyrimidin-4-yl]-3- methyl-indol-5-yl]prop-2-enamide | 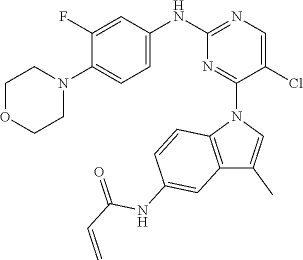 [SMILES: C=CC(=O)Nc1ccc2c(c1)c(C)cn2-c1nc(Nc2ccc(N3CCOCC3)c(F)c2)ncc1Cl] | LCMS m/z = 507 (M + 1, 1 Cl isotopic distribution); ¹H NMR (500 MHz, DMSO-d6) δ: 10.19 (s, 1H), 10.00 (s, 1H), 8.67 (s, 1H), 8.08 (d, 1H, J = 1.5 Hz), 7.87 (br d, 1H, J = 8.9 Hz), 7.68- 7.75 (m, 2H), 7.41-7.45 (m, 1H), 7.36 (dd, 1H, J = 1.8, 8.9 Hz), 6.98 (t, 1H, J = 9.3 Hz), 6.48 (dd, 1H, J = 10.2, 16.9 Hz), 6.27 (dd, 1H, J = 2.0, 16.9 Hz), 5.72-5.77 (m, 1H), 3.69-3.75 (m, 4H), 2.90-2.97 (m, 4H), 2.29 (s, 3H) |
| Example 23 N-[1-[5-chloro-2-(3,4- dimethoxyanilino)pyrimidin-4-yl]-3- methyl-indol-5-yl]prop-2-enamide | 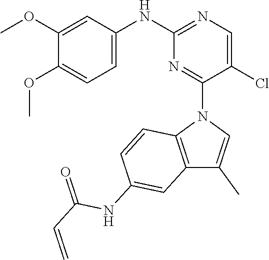 [SMILES: C=CC(=O)Nc1ccc2c(c1)c(C)cn2-c1nc(Nc2ccc(OC)c(OC)c2)ncc1Cl] | LCMS m/z = 464 (M + 1, 1 Cl isotopic distribution); ¹H NMR (500 MHz, DMSO-d6) δ: 10.17 (s, 1H), 9.78 (s, 1H), 8.63 (s, 1H), 8.07 (d, 1H, J = 1.8 Hz), 7.83 (br d, 1H, J = 6.4 Hz), 7.71 (s, 1H), 7.42 (d, 1H, J = 1.8 Hz), 7.40 (d, 1H, J = 1.0 Hz), 7.17 (d, 1H, J = 1.0 Hz), 6.87 (d, 1H, J = 8.9 Hz), 6.47 (dd, 1H, J = 10.1, 17.1 Hz), 6.27 (dd, 1H, J = 2.0, 16.9 Hz), 5.75 (dd, 1H, J = 1.8, 10.1 Hz), 3.71 (s, 3H), 3.62 (br s, 3H), 2.28 (s, 3H) |
| Example 24 N-[1-[5-chloro-2-(4- morpholinoanilino)pyrimidin-4-yl]-3- methyl-indol-5-yl]prop-2-enamide | 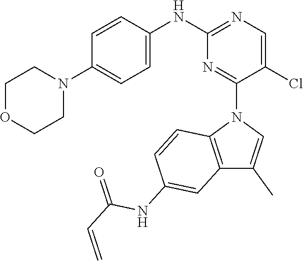 [SMILES: C=CC(=O)Nc1ccc2c(c1)c(C)cn2-c1nc(Nc2ccc(N3CCOCC3)cc2)ncc1Cl] | LCMS m/z = 489 (M + 1) ; ¹H NMR (500 MHz, DMSO-d6) δ: 10.17 (s, 1H), 9.74 (s, 1H), 8.60 (s, 1H), 8.06 (d, J = 1.5 Hz, 1H), 7.84 (br s, 1H), 7.70 (s, 1H), 7.55 (d, J = 9.2 Hz, 2H), 7.41 (d, J = 8.9 Hz, 1H), 6.88 (br d, J = 8.9 Hz, 2H), 6.47 (dd, J = 17.1, 10.1 Hz, 1H), 6.27 (dd, J = 16.9, 2.0 Hz, 1H), 5.73- 5.77 (m, 1H), 3.73 (t, J = 4.6 Hz, 4H), 3.03 (t, J = 4.9 Hz, 4H), 2.28 (s, 3H) |
| Example 25 N-[1-[5-chloro-2-[(1-methylpyrazol- 4-yl)amino]pyrimidin-4-yl]-3-methyl- indol-5-yl]prop-2-enamide | 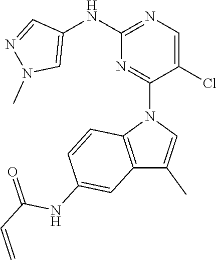 [SMILES: C=CC(=O)Nc1ccc2c(c1)c(C)cn2-c1nc(Nc2cnn(C)c2)ncc1Cl] | LCMS m/z = 408 (M + 1, 1 Cl isotopic distribution); ¹H NMR (DMSO-d6, 500 MHz, 353K) δ: 9.87 (br s, 1H), 9.49 (br s, 1H), 8.54 (s, 1H), 7.99 (d, 1H, J = 1.8 Hz), 7.82 (br d, 1H, J = 8.9 Hz), 7.78 (s, 1H), 7.66 (s, 1H), 7.51 (s, 1H), 7.47 (dd, 1H, J = 2.1, 8.9 Hz), 6.48 (dd, 1H, J = 10.4, 17.1 Hz), 6.24-6.31 (m, 1H), 5.72 (dd, 1H, J = 2.0, 10.2 Hz), 3.78 (s, 3H), 2.31 (s, 3H) |
| Example 26 N-[1-[5-chloro-2-[(3-methoxy-1- methyl-pyrazol-4-yl)amino]pyrimidin- 4-yl]-3-methyl-indol-5-yl]prop-2- enamide | 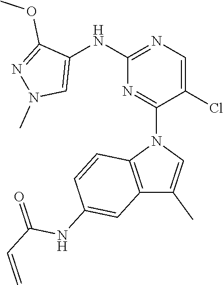 [SMILES: C=CC(=O)Nc1ccc2c(c1)c(C)cn2-c1nc(Nc2cn(C)nc2OC)ncc1Cl] | LCMS m/z = 438 (M + 1, 1 Cl isotopic distribution); ¹H NMR (500 MHz, DMSO-d6) δ: 9.86 (s, 1H), 8.49 (s, 1H), 8.47 (s, 1H), 7.96 (d, 1H, J = 1.8 Hz), 7.82 (d, 1H, J = 8.9 Hz), 7.70 (d, 1H, J = 0.9 Hz), 7.59 (s, 1H), 7.41 (dd, 1H, J = 2.0, 9.0 Hz), 6.47 (dd, 1H, J = 10.2, 16.9 Hz), 6.27 (dd, 1H, J = 1.8, 17.1 Hz), 5.71 (dd, 1H, J = 2.0, 10.2 Hz), 3.83 (s, 3H), 3.67 (s, 3H), 2.28 (d, 3H, J = 0.9 Hz) |
| Example 27 N-[3-[5-chloro-2-(4- morpholinoanilino)pyrimidin-4-yl]-1- methyl-indol-6-yl]prop-2-enamide | 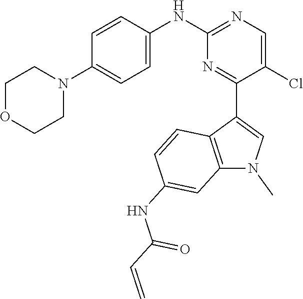 [SMILES: C=CC(=O)Nc1ccc2c(-c3nc(Nc4ccc(N5CCOCC5)cc4)ncc3Cl)cn(C)c2c1] | LCMS m/z = 489 (M + 1, 1 Cl isotopic distribution); ¹H NMR (500 MHz, DMSO-d6) δ: 10.26 (s, 1H) 9.37 (s, 1H) 8.54 - 8.59 (m, 1H) 8.52 (s, 1H) 8.37 (s, 1H) 8.16 (s, 1H) 7.58 (dd, J = 9.1 Hz, 2H) 7.24 (br d, J = 8.5 Hz, 1H) 6.92 (dd, J = 9.1 Hz, 2H) 6.50 (dd, J = 16.7, 10.0 Hz, 1H) 6.28 (dd, J = 16.9, 1.9 Hz, 1H) 5.74 - 5.79 (m, 1H) 3.87 (s, 3H) 3.71 - 3.80 (m, 4H) 3.02 - 3.10 (m, 4H) |
| Example 28 N-[1-[5-chloro-2-[(1-methylimidazol- 4-yl)amino]pyrimidin-4-yl]-3-methyl- indol-5-yl]prop-2-enamide | 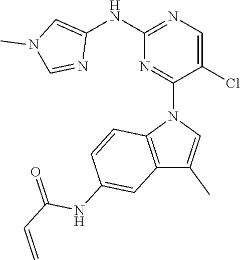 [SMILES: C=CC(=O)Nc1ccc2c(c1)c(C)cn2-c1nc(Nc2cn(C)cn2)ncc1Cl] | LCMS m/z = 408 (M + 1, 1 Cl isotopic distribution; ¹H NMR (500 MHz, DMSO-d6) δ: 10.18 (s, 1H), 10.09 (s, 1H), 8.61 (s, 1H), 8.05 (d, J = 1.71 Hz, 1H), 7.96 (br s, 1H), 7.73 (br s, 1H), 7.49 (br s, 1H), 7.44 (dd, J = 1.81, 8.97 Hz, 1H), 7.17 (s, 1H), 6.48 (dd, J = 10.14, 16.90 Hz, 1H), 6.27 (dd, J = 1.95, 16.95 Hz, 1H), 5.75 (dd, J = 1.95, 10.11 Hz, 1H), 3.60 (s, 3H), 2.29 (s, 3H) |
| Example 29 N-[1-[5-chloro-2-(2-ethoxy-6-methyl- anilino)pyrimidin-4-yl]-3-methyl- indol-5-yl]prop-2-enamide | 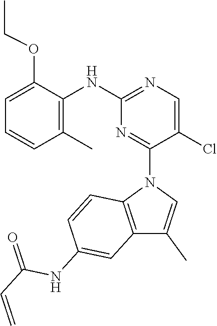 [SMILES: C=CC(=O)Nc1ccc2c(c1)c(C)cn2-c1nc(Nc2c(C)cccc2OCC)ncc1Cl] | LCMS m/z = 462 (M + 1, 1 Cl isotopic distribution); ¹H NMR (500 MHz, DMSO- d6) δ: 10.12 (s, 1H) 10.12 (s, 1H) 8.85 (s, 1H) 8.48 (s, 1H) 7.98 (s, 1H) 7.74 (s, 1H) 7.55 (br s, 1H) 7.15 (t, J = 7.9 Hz, 2H) 6.92 (d, J = 8.2 Hz, 1H) 6.85 (d, J = 7.3 Hz, 1H) 6.45 (dd, J = 16.7, 10.0 Hz, 1H) 6.25 (dd, J = 17.0, 1.8 Hz, 1H) 5.71 - 5.76 (m, 1H) 3.99 (q, J = 6.9, 2H) 2.23 (s, 3H) 2.17 (s, 3H) 1.16 (t, J = 6.9 Hz, 3H) |
| Example 30 N-[3-[5-chloro-2-[(1-methylpyrazol- 4-yl)amino]pyrimidin-4-yl]-1-methyl- indol-6-yl]prop-2-enamide | 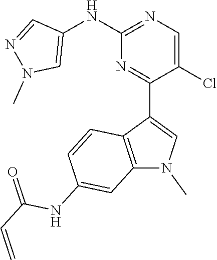 [SMILES: C=CC(=O)Nc1ccc2c(-c3nc(Nc4cnn(C)c4)ncc3Cl)cn(C)c2c1] | LCMS m/z = 408 (M + 1, 1 Cl isotopic distribution); ¹H NMR (500 MHz, DMSO-d6, 353K) δ: 9.97 (br s, 1H), 9.09 (br s, 1H), 8.46 (br d, 1H, J = 8.5 Hz), 8.39 (s, 1H), 8.35 (s, 1H), 8.08 (d, 1H, J = 0.9 Hz), 7.84 (s, 1H), 7.53 (s, 1H), 7.32 (dd, 1H, J = 1.7, 8.7 Hz), 6.50 (dd, 1H, J = 10.2, 16.9 Hz), 6.29 (dd, 1H, J = 2.0, 16.9 Hz), 5.73 (dd, 1H, J = 1.8, 10.4 Hz), 3.87 (s, 3H), 3.82 (s, 3H) |
| Example 31 N-[3-[5-chloro-2-(3,4- dimethoxyanilino)pyrimidin-4-yl]-1- methyl-indol-6-yl]prop-2-enamide | 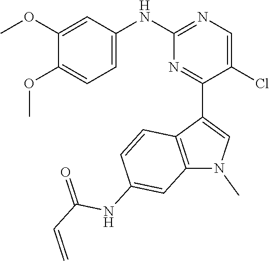 [SMILES: C=CC(=O)Nc1ccc2c(-c3nc(Nc4ccc(OC)c(OC)c4)ncc3Cl)cn(C)c2c1] | LCMS m/z = 464 (M + 1, 1 Cl isotopic distribution); ¹H NMR (500 MHz, DMSO-d6) δ: 10.28 (s, 1H), 9.41 (s, 1H), 8.54 (s, 1H), 8.56 - 8.48 (m, 1H), 8.40 (s, 1H), 8.19 (d, J = 1.2 Hz, 1H), 7.37 (d, J = 2.4 Hz, 1H), 7.22 (br t, J = 7.0 Hz, 2H), 6.91 (d, J = 8.5 Hz, 1H), 6.50 (dd, J = 10.1, 17.1 Hz, 1H), 6.28 (dd, J = 1.8, 16.8 Hz, 1H), 5.76 (dd, J = 1.8, 10.1 Hz, 1H), 3.87 (s, 3H), 3.75 (s, 3H), 3.67 (s, 3H) |
| Example 32 N-[1-[5-cyano-2-[(1-methylpyrazol-4- yl)amino]pyrimidin-4-yl]-3-methyl- indol-5-yl]prop-2-enamide | 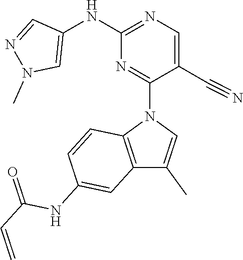 [SMILES: C=CC(=O)Nc1ccc2c(c1)c(C)cn2-c1nc(Nc2cnn(C)c2)ncc1C#N] | LCMS m/z = 399 (M + 1); ¹H NMR (600 MHz, DMSO-d6) δ: 10.48 - 10.42 (m, 2H), 10.24 (d, J = 7.3 Hz, 2H), 8.89 (s, 1H), 8.85 (s, 1H), 8.48 (d, J = 9.0 Hz, 1H), 8.09 (d, J = 1.8 Hz, 1H), 8.07 (d, J = 1.8 Hz, 1H), 8.00 (d, J = 8.8 Hz, 1H), 7.97 (s, 1H), 7.92 (d, J = 1.1 Hz, 1H), 7.85 (s, 1H), 7.80 (d, J = 1.3 Hz, 1H), 7.58 (d, J = 3.7 Hz, 2H), 7.55 (dd, J = 2.0, 9.0 Hz, 1H), 7.44 (dd, J = 2.1, 8.9 Hz, 1H), 6.51 - 6.45 (m, 2H), 6.30 - 6.26 (m, 2H), 5.78 - 5.75 (m, 2H), 3.85 (s, 3H), 3.80 (s, 3H), 2.31 - 2.28 (m, 6H) |
| Example 33 N-[1-[5-cyano-2-(2-ethoxy-6-methyl- anilino)pyrimidin-4-yl]-3-methyl- indol-5-yl]prop-2-enamide | 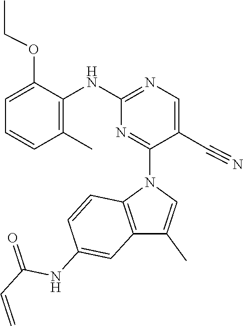 [SMILES: C=CC(=O)Nc1ccc2c(c1)c(C)cn2-c1nc(Nc2c(C)cccc2OCC)ncc1C#N] | LCMS m/z = 453 (M + 1); ¹H NMR (500 MHz, DMSO-d6) δ: 10.25 (br s, 1H), 10.13 (s, 1H), 9.66 (br s, 2H), 8.81 (s, 1H), 8.68 (br s, 1H), 8.58 - 8.51 (m, 1H), 8.08 (br s, 1H), 7.98 (s, 1H), 7.94 (s, 2H), 7.59 - 7.53 (m, 1H), 7.43 (d, J = 9.2 Hz, 1H), 7.31 - 7.24 (m, 1H), 7.21 - 7.14 (m, 1H), 7.05 - 6.98 (m, 2H), 6.93 (br d, J = 7.6 Hz, 2H), 6.89 - 6.84 (m, 1H), 6.43 (br dd, J = 10.1, 16.8 Hz, 2H), 6.31 - 6.21 (m, 2H), 5.74 (br d, J = 10.1 Hz, 2H), 4.04 - 3.90 (m, 4H), 2.30 (br s, 3H), 2.23 (s, 3H), 2.18 (s, 6H), 1.17 (br t, J = 5.5 Hz, 3H), 1.11 (t, J = 6.9 Hz, 3H) |
| Example 34 N-[1-[5-chloro-2-[[1-[1-(oxetan-3-yl)- 4-piperidyl]pyrazol-4- yl]amino]pyrimidin-4-yl]-3-methyl- indol-5-yl]prop-2-enamide | 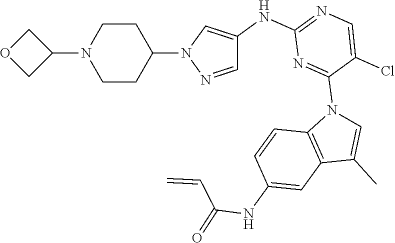 [SMILES: C=CC(=O)Nc1ccc2c(c1)c(C)cn2-c1nc(Nc2cnn(C3CCN(C4COC4)CC3)c2)ncc1Cl] | LCMS m/z = 533 (M + 1, 1 Cl isotopic distribution); ¹H NMR (500 MHz, DMSO- d6) δ: 9.91 (br s, 1H) 9.56 (s, 1H) 8.56 (s, 1H) 7.97 (s, 1H) 7.87 (s, 1H) 7.82 (br d, J = 8.8 Hz, 1H) 7.67 (s, 1H) 7.53 (s, 1H) 7.51 (d, J = 9.3 Hz, 1H) 6.48 (dd, J = 16.9, 10.2 Hz, 1H) 6.27 (dd, J = 16.9, 1.6 Hz, 1H) 5.72 (dd, J = 10.2, 1.6 Hz, 1H) 4.5 (t, J = 6.41 Hz, 2H) 4.44 (t, J = 5.9 Hz, 2H) 4.01 - 4.09 (m, 1H) 3.47 (quin, J = 6.3 Hz, 1H) 2.72 - 2.82 (m, 2H) 2.31 (s, 3H) 2.00 (br t, J = 10.3 Hz, 4H) 1.81 - 1.94 (m, 2H) |
| Example 35 N-[1-[5-chloro-2-[(1,3- dimethylpyrazol-4- yl)amino]pyrimidin-4-yl]-3-methyl- indol-5-yl]prop-2-enamide | 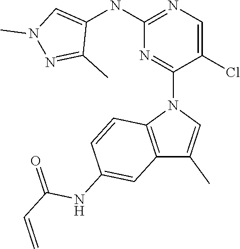 | LCMS m/z = 422 (M + 1, 1 Cl isotopic distribution); 1H NMR (600 MHz, DMSO-d6, 353K) δ: 9.85 (br s, 1H), 8.78 (br s, 1H), 8.47 (s, 1H), 7.95 (d, J = 1.8 Hz, 1H), 7.74 (d, J = 9.0 Hz, 1H), 7.69 (s, 1H), 7.67 (d, J = 1.1 Hz, 1H), 7.40 (dd, J = 9.0, 2.0 Hz, 1H), 6.45 (dd, J = 17.1, 10.3 Hz, 1H), 6.25 (dd, J = 17.0, 1.9 Hz, 1H), 5.70 (dd, J = 10.2, 1.9 Hz, 1H), 3.71 (s, 3H), 2.27 (d, J = 1.3 Hz, 3H), 2.10 (s, 3H) |
| Example 36 N-[3-methyl-1-[5-methyl-2-[(1- methylimidazol-4- yl)amino]pyrimidin-4-yl]indol-5- yl]prop-2-enamide | 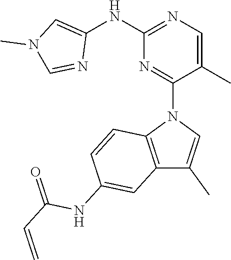 | LCMS m/z = 388 (M + 1); ¹H NMR (600 MHz, DMSO-d6) δ: 9.82 (br s, 1H), 8.96 (br s, 1H), 8.40 (d, J = 0.7 Hz, 1H), 7.97 (d, J = 1.8 Hz, 1H), 7.74 (d, J = 9.0 Hz, 1H), 7.47 (d, J = 1.1 Hz, 1H), 7.41 (dd, J = 8.9, 2.1 Hz, 1H), 7.30 (d, J = 1.3 Hz, 1H), 7.12 (d, J = 1.7 Hz, 1H), 6.46 (dd, J = 17.0, 10.2 Hz, 1H), 6.25 (dd, J = 17.1, 1.8 Hz, 1H), 5.69 (dd, J = 10.2, 1.9 Hz, 1H), 3.58 (s, 3H), 2.30 (d, J = 1.1 Hz, 3H), 2.19 (d, J = 0.6 Hz, 3H) |
| Example 37 N-[1-[5-cyano-2-[(3-methoxy-1- methyl-pyrazol-4-yl)amino]pyrimidin- 4-yl]-3-methyl-indol-5-yl]prop-2- enamide | 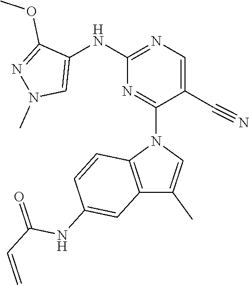 [SMILES: C=CC(=O)Nc1ccc2c(c1)c(C)cn2-c1nc(Nc2cn(C)nc2OC)ncc1C#N] | LCMS m/z = 429 (M + 1); ¹H NMR (500 MHz, DMSO-d6): δ (ppm) 10.23 (s, 1H), 10.20 (s, 2H), 9.75 (s, 1H), 9.56 (s, 2H), 8.79 (s, 2H), 8.78 (s, 1H), 8.61 (d, J = 9.0 Hz, 1H), 8.06 (br s, 3H), 7.94 (d, J = 0.9 Hz, 1H), 7.89-7.91 (m, 4H), 7.77 (s, 1H), 7.69 (s, 2H), 7.53 (dd, J = 9.0, 2.0 Hz, 1H), 7.32 (dd, J = 9.1, 1.9 Hz, 2H), 6.42-6.52 (m, 3H), 6.25-6.30 (m, 3H), 5.74-5.78 (m, 3H), 3.83 (s, 3H), 3.80 (s, 6H), 3.72 (s, 6H), 3.70 (s, 3H), 2.29 (d, J = 0.9 Hz, 3H), 2.26 (d, J = 1.1 Hz, 6H) |
| Example 38 N-[1-[5-cyano-2-(4- morpholinoanilino)pyrimidin-4-yl]-3- methyl-indol-5-yl]prop-2-enamide | 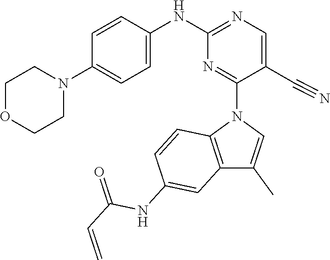 [SMILES: C=CC(=O)Nc1ccc2c(c1)c(C)cn2-c1nc(Nc2ccc(N3CCOCC3)cc2)ncc1C#N] | LCMS m/z = 480 (M + 1); ¹H NMR (500 MHz, DMSO-d6) δ: 10.37-10.18 (m, 2H), 8.85 (s, 1H), 8.52 (s, 0.5H), 8.08- 7.83 (m, 2.5H), 7.60-7.47 (m, 2.5H), 7.32 (s, 0.5H), 6.95 (br s , 2H), 6.47 (dd, J = 10.10, 16.84 Hz, 1H), 6.28 (d, J = 17.01 Hz, 1H), 5.76 (d, J = 10.01 Hz, 1H), 3.77-3.73 (m, 4H), 3.11-3.07 (m, 4H), 2.29 (s, 3H) |
| Example 39 N-[1-[5-cyano-2-(2- ethoxyanilino)pyrimidin-4-yl]-3- methyl-indol-5-yl]prop-2-enamide | 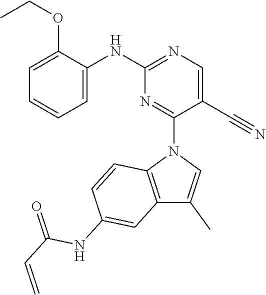 [SMILES: C=CC(=O)Nc1ccc2c(c1)c(C)cn2-c1nc(Nc2ccccc2OCC)ncc1C#N] | LCMS m/z = 439 (M + 1); ¹H NMR (500 MHz, DMSO-d6) δ: 10.18 (s, 1H), 9.70 (s, 1H), 8.82 (s, 1H), 8.02 (s, 1H), 7.91 (s, 1H), 7.72 (br s, 1H), 7.50 (d, J = 6.82 Hz, 1H), 7.31-7.24 (m, 1H), 7.21 (br s, 1H), 7.13 (d, J = 8.23 Hz, 1H), 6.99 (t, J = 7.66 Hz, 1H), 6.45 (dd, J = 10.00, 16.89 Hz, 1H), 6.26 (dd, J = 1.80, 17.05 Hz, 1H), 5.75 (dd, J = 1.91, 10.00 Hz, 1H), 4.04 (q, J = 6.99 Hz, 2H), 2.26 (s, 3H), 1.25-1.18 (m, 3H) |
| Example 40 N-[1-[5-chloro-2-[(1,5- dimethylpyrazol-4- yl)amino]pyrimidin-4-yl]-3-methyl- indol-5-yl]prop-2-enamide | 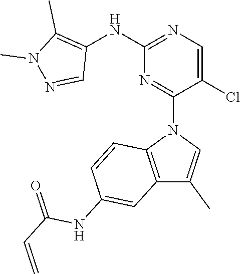 [SMILES: C=CC(=O)Nc1ccc2c(c1)c(C)cn2-c1nc(Nc2cnn(C)c2C)ncc1Cl] | LCMS m/z = 422 (M + 1, 1 Cl isotopic distribution); ¹H NMR (500 MHz, DMSO-d6) δ: 9.89 (br s, 1H), 8.79 (s, 1H), 8.47 (s, 1H), 7.99 (s, 1H), 7.73 - 7.67 (m, 2H), 7.45 (s, 1H), 7.38 (br d, J = 8.2 Hz, 1H), 6.47 (dd, J = 10.4, 17.1 Hz, 1H), 6.27 (dd, J = 1.7, 16.9 Hz, 1H), 5.72 (dd, J = 1.8, 10.4 Hz, 1H), 3.75 (s, 3H), 2.28 (s, 3H), 2.15 (s, 3H) |
| Example 41 N-[1-[5-chloro-2-[(1,3,5- trimethylpyrazol-4- yl)amino]pyrimidin-4-yl]-3-methyl- indol-5-yl]prop-2-enamide | 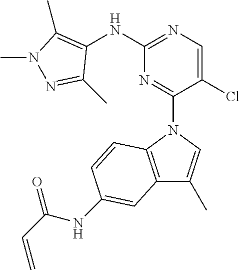 [SMILES: C=CC(=O)Nc1ccc2c(c1)c(C)cn2-c1nc(Nc2c(C)nn(C)c2C)ncc1Cl] | LCMS m/z = 436 (M + 1, 1 Cl isotopic distribution); ¹H NMR (500 MHz, DMSO-d6) δ: 9.89 (br s, 1H), 8.53 (br s, 1H), 8.43 (s, 1H), 7.96 (s, 1H), 7.73 (s, 1H), 7.67 (br s, 1H), 7.35 (br d, J = 8.2 Hz, 1H), 6.46 (dd, J = 10.1, 17.1 Hz, 1H), 6.27 (dd, J = 1.8, 17.1 Hz, 1H), 5.72 (dd, J = 1.8, 10.1 Hz, 1H), 3.68 (s, 3H), 2.27 (s, 3H), 2.07 (s, 3H), 1.99 (s, 3H) |
| Example 42 N-[1-[5-chloro-2-[[1-(1-ethyl-4- piperidyl)pyrazol-4-yl]amino]- pyrimidin-4-yl]-3-methyl-indol-5- yl]prop-2-enamide | 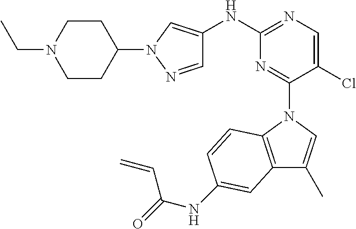 [SMILES: C=CC(=O)Nc1ccc2c(c1)c(C)cn2-c1nc(Nc2cnn(C3CCN(CC)CC3)c2)ncc1Cl] | LCMS m/z = 505 (M + 1, 1 Cl isotopic distribution); ¹H NMR (500 MHz, DMSO- d6) δ: 9.91 (br s, 1H) 9.55 (br s, 1H) 8.55 (s, 1H) 7.98 (s, 1H) 7.86 (s, 1H) 7.82 (br d, J = 8.8 Hz, 1H) 7.66 (s, 1H) 7.52 (s, 1H) 7.50 (dd, J = 8.7, 1.3 Hz, 1H) 6.48 (dd, J = 16.9, 10.2 Hz, 1H) 6.27 (dd, J = 16.9, 1.6 Hz, 1H) 5.72 (dd, J = 10.2, 1.6 Hz, 1H) 3.98 - 4.05 (m, 1H) 2.90 (br d, J = 11.9 Hz, 2H) 2.36 (br d, J = 7.0 Hz, 2H) 2.31 (s, 3H) 2.05 (br t, J = 11.4 Hz, 2H) 1.97 (br d, J = 10.9 Hz, 2H) 1.75 - 1.92 (m, 2H) 1.00 (t, J = 7.1 Hz, 3H) |
| Example 43 N-[3-[5-chloro-2-(pyrazolo[1,5- a]pyrimidin-6-ylamino)pyrimidin-4- yl]-1-methyl-indol-6-yl]prop-2- enamide | 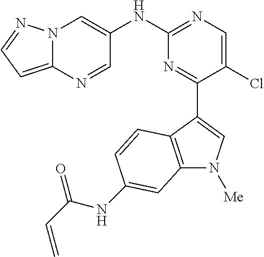 [SMILES: C=CC(=O)Nc1ccc2c(-c3nc(Nc4cnc5ccnn5c4)ncc3Cl)cn(C)c2c1] | LCMS m/z = 445 (M + 1, 1 Cl isotopic distribution); ¹H NMR (500 MHz, DMSO-d6) δ: 10.31 (s, 1H), 10.20 (s, 1H), 9.53 (s, 1H), 8.78 (s, 1H), 8.75 (s, 1H), 8.10 (s, 2H), 7.90 (br s, 1H), 7.74 (s, 1H), 7.40 (br s, 1H), 6.70 (s, 1H), 6.48 (dd, J = 16.5, 10.1 Hz, 1H), 6.27 (d, J = 16.8 Hz, 1H), 5.75 (d, J = 9.5 Hz, 1H), 2.29 (s, 3H) |
| Example 44 N-[3-methyl-1-[5-methyl-2-(4- morpholinoanilino)pyrimidin-4- yl]indol-5-yl]prop-2-enamide | 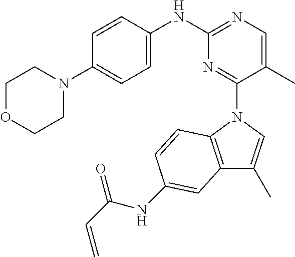 [SMILES: C=CC(=O)Nc1ccc2c(c1)c(C)cn2-c1nc(Nc2ccc(N3CCOCC3)cc2)ncc1C] | LCMS m/z = 469 (M + 1); ¹H NMR (500 MHz, DMSO-d6) δ: 10.15 (s, 1H), 9.48 (s, 1H), 8.47 (s, 1H), 8.07 (d, J = 1.8 Hz, 1H), 7.76 (d, J = 8.8 Hz, 1H), 7.58 (d, J = 1.1 Hz, 1H), 7.52 - 7.49 (m, 1H), 7.39 (dd, J = 8.8, 1.8 Hz, 1H), 7.24 (br d, J = 6.4 Hz, 1H), 6.78 (d, J = 8.6 Hz, 1H), 6.48 (dd, J = 17.0, 10.2 Hz, 1H), 6.27 (dd, J = 16.9, 2.0 Hz, 1H), 5.76 - 5.74 (m, 1H), 3.71 - 3.68 (m, 4H), 3.63 (s, 3H), 2.93 - 2.86 (m, 4H), 2.30 (d, J = 0.9 Hz, 3H), 2.23 (S, 3H) |
| Example 45 N-[3-[5-chloro-2-[(6-morpholino-3- pyridyl)amino]pyrimidin-4-yl]-1- methyl-indol-6-yl]prop-2-enamide | 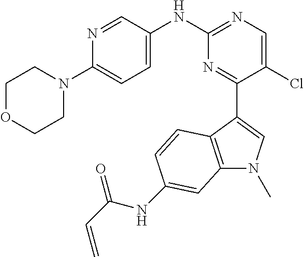 [SMILES: C=CC(=O)Nc1ccc2c(-c3nc(Nc4ccc(N5CCOCC5)nc4)ncc3Cl)cn(C)c2c1] | LCMS m/z = 490 (M + 1, 1 Cl isotopic distribution); ¹H NMR (500 MHz, DMSO-d6) δ: 10.24 (s, 1H), 9.37 (s, 1H), 8.53 (s, 1H), 8.48 (br s, 1H), 8.41 (d, J = 2.60 Hz, 1H), 8.37 (s, 1H), 8.15 (d, J = 1.34 Hz, 1H), 7.90 (dd, J = 2.61, 9.08 Hz, 1H), 7.24 (d, J = 8.35 Hz, 1H), 6.85 (d, J = 9.12 Hz, 1H), 6.50 (dd, J = 10.02, 16.90 Hz, 1H), 6.28 (dd, J = 1.90, 16.90 Hz, 1H), 5.76 (dd, J = 1.75, 10.17 Hz, 1H), 3.87 (s, 3H), 3.75-3.70 (m, 4H), 3.41-3.37 (m, 4H) |
| Example 46 N-[1-[2-(3-methoxy-4-morpholino- anilino)-5-methyl-pyrimidin-4-yl]-3- methyl-indol-5-yl]prop-2-enamide | 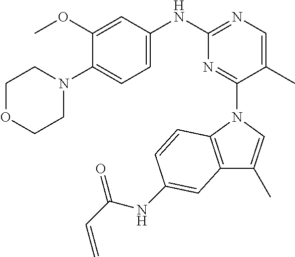 [SMILES: C=CC(=O)Nc1ccc2c(c1)c(C)cn2-c1nc(Nc2ccc(N3CCOCC3)c(OC)c2)ncc1C] | LCMS m/z = 499 (M + 1); ¹H NMR (500 MHz, DMSO-d6) δ: 10.15 (s, 1H), 9.48 (s, 1H), 8.47 (s, 1H), 8.07 (d, J = 1.8 Hz, 1H), 7.76 (d, J = 8.8 Hz, 1H), 7.58 (d, J = 1.1 Hz, 1H), 7.52 - 7.49 (m, 1H), 7.39 (dd, J = 8.8, 1.8 Hz, 1H), 7.24 (br d, J = 6.4 Hz, 1H), 6.78 (d, J = 8.6 Hz, 1H), 6.48 (dd, J = 17.0, 10.2 Hz, 1H), 6.27 (dd, J = 16.9, 2.0 Hz, 1H), 5.76 - 5.74 (m, 1H), 3.71 - 3.68 (m, 4H), 3.63 (s, 3H), 2.93 - 2.86 (m, 4H), 2.30 (d, J = 0.9 Hz, 3H), 2.23 (s, 3H) |
| Example 47 N-[1-[5-chloro-2-([1,2,4]triazolo[1,5- a]pyridin-6-ylamino)pyrimidin-4-yl]- 3-methyl-indol-5-yl]prop-2-enamide | 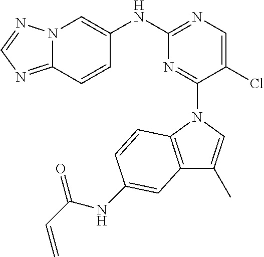 [SMILES: C=CC(=O)Nc1ccc2c(c1)c(C)cn2-c1nc(Nc2ccc3ncnn3c2)ncc1Cl] | LCMS m/z = 445 (M + 1, 1 Cl isotopic distribution); ¹H NMR (600 MHz, DMSO-d6) δ: 10.00 (br s, 2H), 9.91 (br s, 2H), 9.81 (br s, 1H), 9.43 (s, 2H), 9.16-9.19 (m, 1H), 8.73 (s, 2H), 8.47 (s, 1H), 8.41 (s, 1H), 8.34 (s, 2H), 8.13 (d, J = 8.8 Hz, 1H), 8.03 (d, J = 1.7 Hz, 2H), 7.96-7.98 (m, 1H), 7.92-7.95 (m, 1H), 7.86-7.90 (m, 6H), 7.83-7.84 (m, 1H), 7.78 (d, J = 9.4 Hz, 2H), 7.71 (d, J = 1.3 Hz, 2H), 7.44 (dd, J = 8.8, 2.0 Hz, 2H), 7.10-7.13 (m, 1H), 6.43-6.51 (m, 3H), 6.23-6.30 (m, 3H), 5.73 (dd, J = 10.2, 1.9 Hz, 2H), 5.70 (dd, J = 10.1, 2.0 Hz, 1H), 2.33 (d, J = 1.3 Hz, 6H), 2.24 (s, 3H) ppm |
| Example 48 N-[3-[5-chloro-2-[3-methoxy-4-(4- methylpiperazin-1-yl)anilino]- pyrimidin-4-yl]-1-methyl-indol-6- yl]prop-2-enamide | 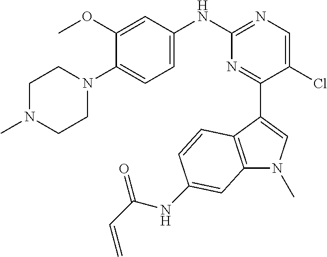 [SMILES: C=CC(=O)Nc1ccc2c(-c3nc(Nc4ccc(N5CCN(C)CC5)c(OC)c4)ncc3Cl)cn(C)c2c1] | LCMS m/z = 532 (M + 1, 1 Cl isotopic distribution); ¹H NMR (600 MHz, DMSO-d6) δ: 10.26 (s, 1H), 9.42 (s, 1H), 8.59 (br s, 1H), 8.53 (s, 1H), 8.41 (s, 1H), 8.18 (d, 1H, J = 1.7 Hz), 7.37 (d, 1H, J = 2.2 Hz), 7.22-7.28 (m, 2H), 6.83 (d, 1H, J = 8.6 Hz), 6.50 (dd, 1H, J = 10.2, 17.0 Hz), 6.28 (dd, 1H, J = 1.9, 17.0 Hz), 5.75-5.78 (m, 1H), 3.88 (s, 3H), 3.70 (s, 3H), 2.85-3.05 (m, 4H), 2.35-2.55 (m, 4H), 2.23 (s, 3H) |
| Example 49 N-[3-methyl-1-[5-methyl-2-[(2- methylpyrazol-3-yl)amino]-pyrimidin- 4-yl]indol-5-yl]prop-2-enamide | 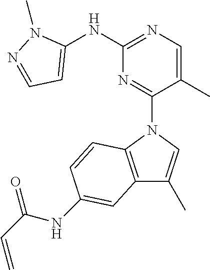 [SMILES: C=CC(=O)Nc1ccc2c(c1)c(C)cn2-c1nc(Nc2ccnn2C)ncc1C] | LCMS m/z = 388 (M + 1); ¹H NMR (600 MHz, DMSO-d6) δ: 10.14 (s, 1H), 9.37 (s, 1H), 8.44 (s, 1H), 8.04 (d, J = 1.8 Hz, 1H), 7.68 (d, J = 8.8 Hz, 1H), 7.57 (d, J = 1.1 Hz, 1H), 7.25-7.43 (m, 2H), 6.47 (dd, J = 17.0, 10.2 Hz, 1H), 6.26 (dd, J = 17.0, 1.9 Hz, 1H), 6.23 (d, J = 1.8 Hz, 1H), 5.74 (dd, J = 10.1, 2.2 Hz, 1H), 3.67 (s, 3H), 2.28 (d, J = 1.1 Hz, 3H), 2.25 (s, 3H) |
| Example 50 N-[3-[5-chloro-2-[4-(4- methylpiperazin-1-yl)anilino]- pyrimidin-4-yl]-1-methyl-indol-6- yl]prop-2-enamide | 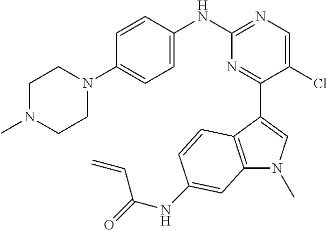 [SMILES: C=CC(=O)Nc1ccc2c(-c3nc(Nc4ccc(N5CCN(C)CC5)cc4)ncc3Cl)cn(C)c2c1] | LCMS m/z = 502 (M + 1, 1 Cl isotopic distribution); ¹H NMR (500 MHz, DMSO-d6) δ: 10.32 (s, 1H) 9.41 (s, 1H) 8.52 (s, 1H) 8.49 (br s, 1H) 8.39 (s, 1H) 8.13 (s, 1H) 7.61 (br d, J = 8.8 Hz, 2H) 7.29 (br d, J = 8.5 Hz, 1H) 6.99 (d, J = 8.8 Hz, 2H) 6.51 - 6.59 (m, 1H) 6.25 - 6.30 (m, 1H) 5.76 (d, J = 11.3 Hz, 1H) 3.87 (s, 3H) 3.75 (br d, J = 12.8 Hz, 2H) 3.42 - 3.61 (m, 2H) 3.11 - 3.26 (m, 2H) 3.01 (br t, J = 12.0 Hz, 2H) 2.85 (br d, J = 3.3 Hz, 3H) |
| Example 51 N-[3-[5-chloro-2-(3-methoxy-4- morpholino-anilino)pyrimidin-4-yl]-1- methyl-indol-6-yl]prop-2-enamide | 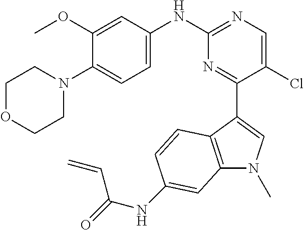 [SMILES: C=CC(=O)Nc1ccc2c(-c3nc(Nc4ccc(N5CCOCC5)c(OC)c4)ncc3Cl)cn(C)c2c1] | LCMS m/z = 519 (M + 1, 1 Cl isotopic distribution); ¹H NMR (500 MHz, DMSO-d6) δ: 10.25 (s, 1H) 9.44 (s, 1H) 8.56 (br.s, 1H) 8.53 (s, 1H) 8.41 (s, 1H) 8.17 (s, 1H) 7.39 (d, J = 2.4 Hz, 1H) 7.28 (br d, J = 8.2 Hz, 1H) 7.24 (br d, J = 8.5 Hz, 1H) 6.84 (d, J = 8.8 Hz, 1H) 6.50 (dd, J = 16.7, 10.1 Hz, 1H) 6.28 (dd, J = 16.9, 1.98 Hz, 1H) 5.76 (dd, J = 10.1, 1.9 Hz, 1H) 3.88 (s, 3H) 3.70 - 3.76 (m, 4H) 3.69 (s, 3H) 2.88 - 2.98 (m, 4H) |
| Example 52 N-[1-methyl-3-[5-methyl-2-(4- morpholinoanilino)pyrimidin-4- yl]indol-6-yl]prop-2-enamide | 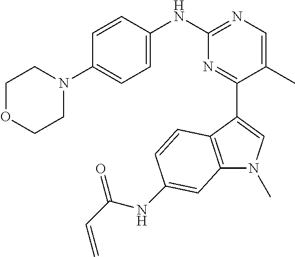 [SMILES: C=CC(=O)Nc1ccc2c(-c3nc(Nc4ccc(N5CCOCC5)cc4)ncc3C)cn(C)c2c1] | LCMS m/z = 469 (M + 1); ¹H NMR (500 MHz, DMSO-d6) δ: 10.22 (s, 1H), 9.03 (s, 1H), 8.56 (d, J = 8.63 Hz, 1H), 8.20 (s, 1H), 8.15 (s, 1H), 8.00 (s, 1H), 7.67- 7.63 (m, 2H), 7.21 (d, J = 8.66 Hz, 1H), 6.90-6.86 (m, 2H), 6.50 (dd, J = 10.19, 16.95 Hz, 1H), 6.28 (dd, J = 1.72, 16.99 Hz, 1H), 5.76 (dd, J = 1.51, 10.03 Hz, 1H), 3.86 (s, 3H), 3.76-3.73 (m, 4H), 3.05-3.02 (m, 4H), 2.36 (s, 3H) |
| Example 53 N-[1-methyl-3-[2-(4-morpholino- anilino)pyrimidin-4-yl]indol-6- yl]prop-2-enamide | 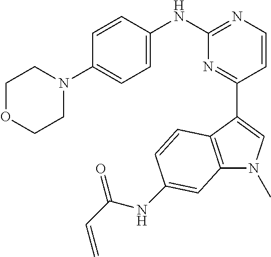 [SMILES: C=CC(=O)Nc1ccc2c(-c3ccnc(Nc4ccc(N5CCOCC5)cc4)n3)cn(C)c2c1] | LCMS m/z = 455 (M + 1); ¹H NMR (500 MHz, DMSO-d6) δ: 10.23 (s, 1H), 9.14 (s, 1H), 8.51 (br d, 1H, J = 8.2 Hz), 8.26 (d, 1H, J = 5.2 Hz), 8.23 (s, 1H), 8.14 (s, 1H), 7.65 (d, 2H, J = 8.9 Hz), 7.25 (dd, 1H, J = 1.2, 8.5 Hz), 7.10 (d, 1H, J = 5.5 Hz), 6.93 (d, 2H, J = 9.2 Hz), 6.49 (dd, 1H, J = 10.1, 17.1 Hz), 6.28 (dd, 1H, J = 2.0, 16.9 Hz), 5.76 (dd, 1H, J = 1.8, 10.1 Hz), 3.83 (s, 3H), 3.74-3.77 (m, 4H), 3.05-3.07 |
| Example 54 N-[3-methyl-1-[2-(4- morpholinoanilino)pyrimidin-4- yl]indol-5-yl]prop-2-enamide | 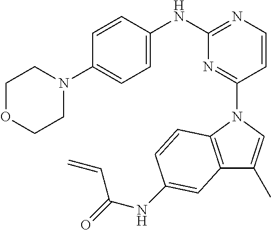 [SMILES: C=CC(=O)Nc1ccc2c(c1)c(C)cn2-c1ccnc(Nc2ccc(N3CCOCC3)cc2)n1] | LCMS m/z = 455 (M + 1); ¹H NMR (500 MHz, DMSO-d6) δ: 10.17 (s, 1H) 9.39 (s, 1H) 8.69 (br s, 1H) 8.36 (d, J = 5.8 Hz, 1H) 8.05 (d, J = 1.8 Hz, 1H) 7.91 (s, 1H) 7.58 (br d, J = 8.5 Hz, 2H) 7.42 (br d, J = 8.2 Hz, 1H) 7.03 (d, J = 5.8 Hz, 1H) 6.95 (d, J = 9.1 Hz, 2H) 6.47 (dd, J = 16.9, 10.2 Hz, 1H) 6.27 (dd, J = 16.9, 1.9 Hz, 1H) 5.73 - 5.77 (m, 1H) 3.72 - 3.79 (m, 4H) 3.03 - 3.12 (m, 4H) 2.27 (s, 3H) |
| Example 55 N-[1-[5-chloro-2-[(2-methylpyrazol- 3-yl)amino]pyrimidin-4-yl]-3-methyl- indol-5-yl]prop-2-enamide | 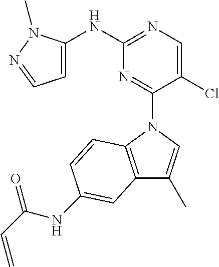 [SMILES: C=CC(=O)Nc1ccc2c(c1)c(C)cn2-c1nc(Nc2ccnn2C)ncc1Cl] | LCMS m/z = 408 (M + 1, 1 Cl isotopic distribution); ¹H NMR (500 MHz, DMSO-d6) δ: 10.17 (s, 1H), 9.53 - 9.86 (s, 1H), 8.63 (s, 1H), 8.04 (d, J = 1.83 Hz, 1H), 7.64 - 7.81 (m, 2H), 7.28 - 7.43 (m, 2H), 6.46 (dd, J = 16.94, 10.22 Hz, 1H), 6.28 (d, J = 2.14 Hz, 1H), 6.24 (s, 1H), 5.74 (dd, J = 10.07, 1.83 Hz, 1H), 3.67 (s, 3H), 2.27 (s, 3H) |
| Example 56 N-[1-[5-chloro-2-[(2,5- dimethylpyrazol-3-yl)amino]- pyrimidin-4-yl]-3-methyl-indol-5- yl]prop-2-enamide | 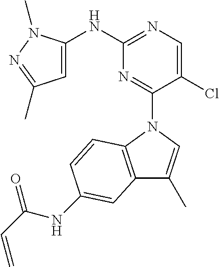 [SMILES: C=CC(=O)Nc1ccc2c(c1)c(C)cn2-c1nc(Nc2cc(C)nn2C)ncc1Cl] | LCMS m/z = 422 (M + 1, 1 Cl isotopic distribution); 1H NMR (500 MHz, DMSO-d6) δ: 10.17 (s, 1H), 9.66 (s, 1H), 8.61 (s, 1H), 8.05 (d, J = 1.83 Hz, 1H), 7.76 (br d, J = 0.92 Hz, 2H), 7.25 - 7.45 (m, 2H), 6.47 (dd, J = 16.78, 10.07 Hz, 1H), 6.26 (dd, J = 16.94, 1.98 Hz, 1H), 6.04 (s, 1H), 5.75 (dd, J = 10.22, 1.98 Hz, 1H), 3.58 (s, 3H), 2.27 (s, 3H), 2.13 (s, 3H) |
| Example 57 N-[3-[5-fluoro-2-(4-morpholino- anilino)pyrimidin-4-yl]-1-methyl- indol-6-yl]prop-2-enamide | 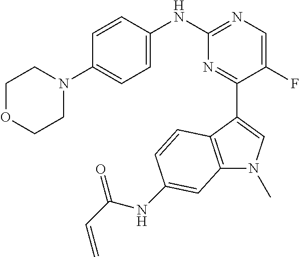 [SMILES: C=CC(=O)Nc1ccc2c(-c3nc(Nc4ccc(N5CCOCC5)cc4)ncc3F)cn(C)c2c1] | LCMS m/z = 472 (M + 1); ¹H NMR (500 MHz, DMSO-d6) δ: 10.26 (s, 1H), 9.29 (s, 1H), 8.65 (d, J = 8.50 Hz, 1H), 8.36 (d, J = 3.93 Hz, 1H), 8.19-8.17 (m, 2H), 7.64-7.59 (m, 2H), 7.30-7.26 (m, 1H), 7.00 (br s, 2H), 6.50 (dd, J = 10.06, 16.90 Hz, 1H), 6.28 (dd, J = 1.95, 16.90 Hz, 1H), 5.77 (dd, J = 1.88, 10.03 Hz, 1H), 3.87 (s, 3H), 3.80-3.76 (m, 4H), 3.16-3.08 (m, 4H) |
| Example 58 N-[3-[5-chloro-2-[4-[2- (dimethylamino)ethyl-methyl-amino]- 3-methoxy-anilino]-pyrimidin-4-yl]-1- methyl-indol-6-yl]prop-2-enamide | 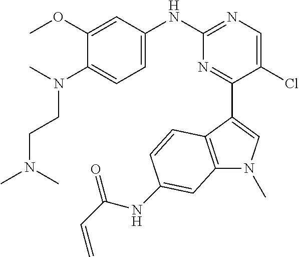 [SMILES: C=CC(=O)Nc1ccc2c(-c3nc(Nc4ccc(N(C)CCN(C)C)c(OC)c4)ncc3Cl)cn(C)c2c1] | LCMS m/z = 534 (M + 1, 1 Cl isotopic distribution); ¹H NMR (600 MHz, DMSO-d6) δ: 10.26 (s, 1H), 9.39 (s, 1H), 8.53 (s, 1H), 8.40 (s, 1H), 8.30 (s, 1H), 8.16 (s, 1H), 7.34 (s, 1H), 7.24 (d, J = 8.3 Hz, 2H), 6.84 (d, J = 8.4 Hz, 1H), 6.50 (dd, J = 17.1, 10.3 Hz, 1H), 6.26-6.31 (m, 1H), 5.76 (d, J = 10.6 Hz, 1H), 3.88 (s, 3H), 3.70 (s, 3H), 3.06 (t, J = 7.0 Hz, 2H), 2.70 (s, 3H), 2.39 (t, J = 7.2 Hz, 2H), 2.15 (s, 6H) |
| Example 59 N-[3-[5-chloro-2-[[6-(4- methylpiperazin-1-yl)-3- pyridyl]amino]pyrimidin-4-yl]-1- methyl-indol-6-yl]prop-2-enamide | 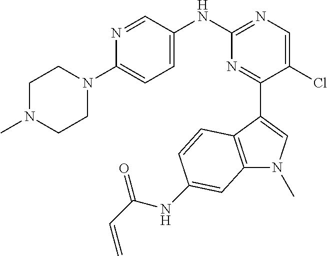 [SMILES: C=CC(=O)Nc1ccc2c(-c3nc(Nc4ccc(N5CCN(C)CC5)nc4)ncc3Cl)cn(C)c2c1] | LCMS m/= 503 (M + 1, 1 Cl isotopic distribution); ¹H NMR (500 MHz, DMSO-d6) δ: 10.28 (s, 1H), 9.39 (s, 1H), 8.53 (s, 1H), 8.49 (br s, 1H), 8.42 (s, 1H), 8.37 (s, 1H), 8.13 (s, 1H), 7.92 (dd, J = 2.4, 8.9 Hz, 1H), 7.27 (br d, J = 8.5 Hz, 1H), 6.91 (d, J = 8.9 Hz, 1H), 6.55 - 6.48 (m, 1H), 6.31 - 6.26 (m, 1H), 5.79 - 5.74 (m, 1H), 3.87 (s, 3H), 3.81 (br s, 4H), 3.02 (br s, 4H), 2.50 (s, 3H) |
| Example 60 N-[1-[5-chloro-2-(4- morpholinoanilino)-pyrimidin-4-yl]- 3,6-dimethyl-indol-5-yl]prop-2- enamide | 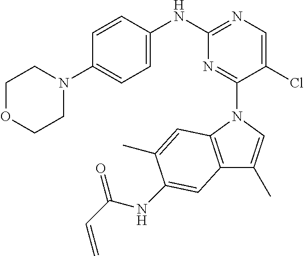 [SMILES: C=CC(=O)Nc1cc2c(C)cn(-c3nc(Nc4ccc(N5CCOCC5)cc4)ncc3Cl)c2cc1C] | LCMS m/z = 505 (M + 1, 1 Cl isotopic distribution); ¹H NMR (500 MHz, DMSO-d6) δ: 9.74 (s, 1H), 9.53 (s, 1H), 8.60 (s, 1H), 7.79 (s, 1H), 7.63 (d, J = 3.1 Hz, 2H), 7.57 - 7.53 (m, J = 8.9 Hz, 2H), 6.91 - 6.85 (m, J = 8.9 Hz, 2H), 6.60 - 6.52 (m, 1H), 6.25 (dd, J = 1.8, 17.1 Hz, 1H), 5.75 (dd, J = 1.8, 10.1 Hz, 1H), 3.75 - 3.70 (m, 4H), 3.06 - 3.00 (m, 4H), 2.74 - 2.52 (m, 1H), 2.29 (s, 6H) |
| Example 61 N-[1-[5-fluoro-2-[(1-methylpyrazol-4- yl)amino]pyrimidin-4-yl]-3-methyl- indol-5-yl]prop-2-enamide | 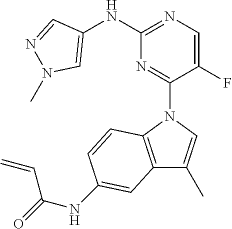 [SMILES: C=CC(=O)Nc1ccc2c(c1)c(C)cn2-c1nc(Nc2cnn(C)c2)ncc1F] | LCMS m/z = 392 (M + 1); ¹H NMR (500 MHz, DMSO-d6) δ: 10.21 (s, 1H) 9.56 (s, 1H) 8.53 (d, J = 4.70 Hz, 1H) 8.12-8.81 (br s, 1H) 8.07 (d, J = 1.9 Hz, 1H) 7.85 (s, 1H) 7.66 (br s, 1H) 7.f49 (s, 1H) 7.45 (br s, 1H) 6.47 (dd, J = 16.9, 10.2 Hz, 1H) 6.26 (dd, J = 16.8, 2.0 Hz, 1H) 5.74 (dd, J = 10.0, 2,1 Hz, 1H) 3.80 (s, 3H) 2.27 (d, J = 1.2 Hz, 3H) |
| Example 62 N-[3-[5-chloro-2-[(2-methylpyrazol- 3-yl)amino]pyrimidin-4-yl]-1-methyl- indol-6-yl]prop-2-enamide | 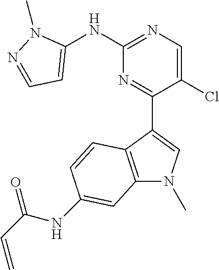 [SMILES: C=CC(=O)Nc1ccc2c(-c3nc(Nc4ccnn4C)ncc3Cl)cn(C)c2c1] | LCMS m/z = 408 (M + 1, 1 Cl isotopic distribution); ¹H NMR (500 MHz, DMSO-d6) δ: 10.23 (s, 1H), 9.31 (s, 1H), 8.58 (s, 1H), 8.40 (s, 1H), 8.15 (d, 1H, J = 1.5 Hz), 7.98 (br s, 1H), 7.45 (d, 1H, J = 1.8 Hz), 7.11 (br d, 1H, J = 7.9 Hz), 6.48 (dd, 1H, J = 10.2, 16.9 Hz), 6.27 (dd, 1H, J = 2.0, 16.9 Hz), 6.21 (d, 1H, J = 1.8 Hz), 5.74-5.77 (m, 1H), 3.86 (s, 3H), 3.63 (s, 3H) |
| Example 63 N-[3-[5-chloro-2-[(2,5- dimethylpyrazol-3-yl)amino]- pyrimidin-4-yl]-1-methyl-indol-6- yl]prop-2-enamide | 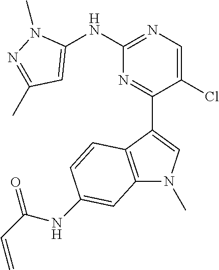 [SMILES: C=CC(=O)Nc1ccc2c(-c3nc(Nc4cc(C)nn4C)ncc3Cl)cn(C)c2c1] | LCMS m/z = 422 (M + 1, 1 Cl isotopic distribution); ¹H NMR (500 MHz, DMSO-d6) δ: 10.24 (s, 1H), 9.30 (s, 1H), 8.58 (s, 1H), 8.39 (s, 1H), 8.17 (s, 1H), 8.11 (br s, 1H), 7.13 (br d, 1H, J = 8.2 Hz), 6.49 (dd, 1H, J = 10.1, 16.8 Hz), 6.28 (dd, 1H, J = 2.0, 16.9 Hz), 6.03 (s, 1H), 5.74-5.77 (m, 1H), 3.86 (s, 3H), 3.56 (s, 3H), 2.20 (s, 3H) |
| Example 64 N-[1-[2-[(2,5-dimethylpyrazol-3- yl)amino]-5-methyl-pyrimidin-4-yl]- 3-methyl-indol-5-yl]prop-2-enamide | 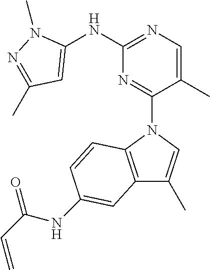 [SMILES: C=CC(=O)Nc1ccc2c(c1)c(C)cn2-c1nc(Nc2cc(C)nn2C)ncc1C] | LCMS m/z = 402 (M + 1); ¹H NMR (500 MHz, DMSO-d6) δ: 10.13 (s, 1H), 9.31 (s, 1H), 8.42 (s, 1H), 8.04 (d, J = 1.89 Hz, 1H), 7.71 (d, J = 8.87 Hz, 1H), 7.57 (s, 1H), 7.33 (dd, J = 1.72, 8.96 Hz, 1H), 6.47 (dd, J = 10.06, 16.97 Hz, 1H), 6.26 (dd, J = 1.95, 16.97 Hz, 1H), 6.03 (s, 1H), 5.74 (dd, J = 1.91, 10.10 Hz, 1H), 3.58 (s, 3H), 2.27 (s, 3H), 2.24 (s, 3H), 2.10 (s, 3H) |
| Example 65 N-[1-[5-chloro-2-[4-[(4- methylpiperazin-1-yl)methyl]- anilino]pyrimidin-4-yl]-3-methyl- indol-5-yl]prop-2-enamide | 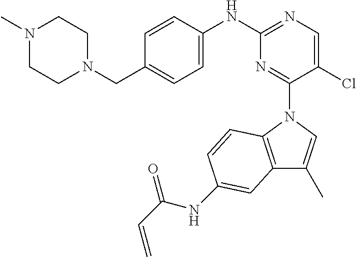 [SMILES: C=CC(=O)Nc1ccc2c(c1)c(C)cn2-c1nc(Nc2ccc(CN3CCN(C)CC3)cc2)ncc1Cl] | LCMS m/z = 516 (M + 1, 1 Cl isotopic distribution); ¹H NMR (500 MHz, DMSO-d6) δ: 10.18 (s, 1H), 9.94 (s, 1H), 8.65 (s, 1H), 8.05 (d, J = 1.8 Hz, 1H), 7.86 (br d, J = 8.9 Hz, 1H), 7.71 (s, 1H), 7.65 (d, J = 8.5 Hz, 2H), 7.42 (br d, J = 8.9 Hz, 1H), 7.18 (d, J = 8.5 Hz, 2H), 6.48 (dd, J = 16.9, 10.2 Hz, 1H), 6.27 (dd, J = 16.9, 2.0 Hz, 1H), 5.75 (dd, J = 10.2, 2.0 Hz, 1H), 3.38 (s, 2H), 2.29 (s, 3H), 2.17-2.41 (m, 8H), 2.13 (s, 3H) |
| Example 66 N-[1-[5-chloro-2-[3-methyl-4- (morpholinomethyl)anilino]pyrimidin- 4-yl]-3-methyl-indol-5-yl]prop-2- enamide | 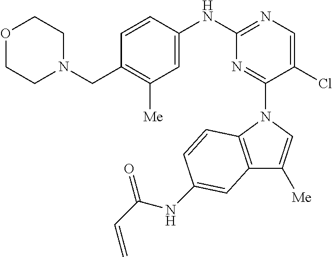 [SMILES: C=CC(=O)Nc1ccc2c(c1)c(C)cn2-c1nc(Nc2ccc(CN3CCOCC3)c(C)c2)ncc1Cl] | LCMS m/z = 517 (M + 1, 1 Cl isotopic distribution); ¹H NMR (500 MHz, DMSO-d6) δ: 10.19 (s, 1H), 9.87 (s, 1H), 8.65 (s, 1H), 8.05 (d, J = 1.8 Hz, 1H), 7.90 (d, J = 8.9 Hz, 1H), 7.71 (s, 1H), 7.58 (s, 1H), 7.41-7.47 (m, 2H), 7.10 (d, J = 8.2 Hz, 1H), 6.48 (dd, J = 17.1, 10.1 Hz, 1H), 6.27 (dd, J = 16.9, 2.0 Hz, 1H), 5.75 (dd, J = 10.2, 2.0 Hz, 1H), 3.53 (br t, J = 4.1 Hz, 4H), 3.36 (s, 2H), 2.33 (br s, 4H), 2.28 (s, 3H), 2.27 (s, 3H) |
| Example 67 N-[1-[5-fluoro-2-(4-morpholino- anilino)pyrimidin-4-yl]-3-methyl- indol-5-yl]prop-2-enamide | 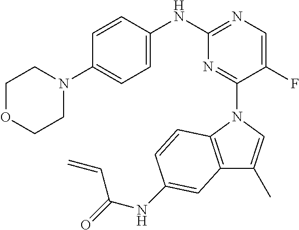 [SMILES: C=CC(=O)Nc1ccc2c(c1)c(C)cn2-c1nc(Nc2ccc(N3CCOCC3)cc2)ncc1F] | LCMS m/z = 473 (M + 1); ¹H NMR (300 MHz, DMSO-d6) δ: 10.20 (s, 1H) 9.50 (s, 1H) 8.53 (d, J = 4.7 Hz, 1H) 8.32-8.48 (m, 1H) 8.06 (d, J = 1.7 Hz, 1H) 7.68 (s, 1H) 7.55 (d, J = 9.0 Hz, 2H) 7.44 (br d, J = 8.8 Hz, 1H) 6.94 (br d, J = 9.0 Hz, 2H) 6.47 (dd, J = 16.9, 10.0 Hz, 1H) 6.26 (dd, J = 16.9, 2.1 Hz, 1H) 5.72 - 5.78 (m, 1H) 3.69 - 3.80 (m, 4H) 2.96 - 3.18 (m, 4H) 2.26 (s, 3H) |
| Example 68 N-[1-[5-chloro-2-[3-fluoro-4- (morpholinomethyl)anilino]pyrimidin- 4-yl]-3-methyl-indol-5-yl]prop-2- enamide | 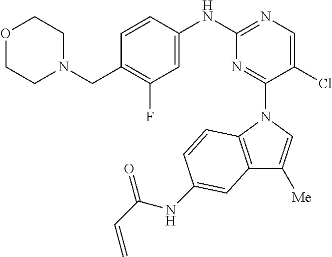 [SMILES: C=CC(=O)Nc1ccc2c(c1)c(C)cn2-c1nc(Nc2ccc(CN3CCOCC3)c(F)c2)ncc1Cl] | LCMS m/z = 521 (M + 1); ¹H NMR (DMSO-d6, 500 MHz) δ 10.19 (s, 1H), 10.18 (s, 1H), 8.72 (s, 1H), 8.07 (d, J = 1.81 Hz, 1H), 7.89 (d, J = 7.88 Hz, 1H), 7.76-7.71 (m, 2H), 7.46-7.40 (m, 2H), 7.27 (t, J = 8.48 Hz, 1H), 6.48 (dd, J = 10.07, 16.90 Hz, 1H), 6.27 (dd, J = 1.91, 16.99 Hz, 1H), 5.76 (dd, J = 1.80, 10.20 Hz, 1H), 3.56-3.53 (m, 4H), 3.44 (s, 2H), 2.37-2.33 (m, 4H), 2.29 (s, 3H). |
| Example 69 N-[1-[5-fluoro-2-[(2-methylpyrazol-3- yl)amino]pyrimidin-4-yl]-3-methyl- indol-5-yl]prop-2-enamide | 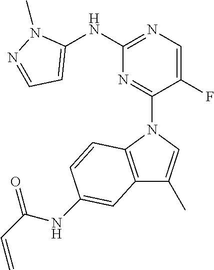 [SMILES: C=CC(=O)Nc1ccc2c(c1)c(C)cn2-c1nc(Nc2ccnn2C)ncc1F] | LCMS m/z = 392 (M + 1); ¹H NMR (DMSO-d6, 500 MHz): δ 10.19 (s, 1H), 9.44 (s, 1H), 8.55 (d, J = 4.9 Hz, 1H), 8.00-8.09, (m, 2H), 7.69 (s, 1H), 7.44 (d, J = 1.8 Hz, 1H), 7.32 (br d, J = 8.5 Hz, 1H), 6.46 (dd, J = 17.1, 10.1 Hz, 1H), 6.27 (dd, J = 17.1, 1.8 Hz, 1H), 6.22 (d, J = 1.8 Hz, 1H), 5.75 (dd, J = 10.2, 2.0 Hz, 1H), 3.66 (s, 3H), 2.26 (s, 3H) |
| Example 70 N-[1-[5-chloro-2-[(3-cyano-1-methyl- pyrazol-4-yl)amino]pyrimidin-4-yl]-3- methyl-indol-5-yl]prop-2-enamide | 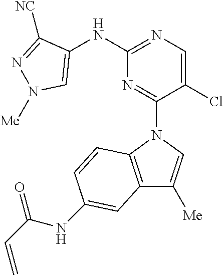 [SMILES: C=CC(=O)Nc1ccc2c(c1)c(C)cn2-c1nc(Nc2cn(C)nc2C#N)ncc1Cl] | LCMS m/z = 433 (M + 1); ¹H NMR (DMSO- d6, 600 MHz) δ 10.18 (s, 1H), 10.15 (s, 1H), 9.92 (br s, 1H), 9.46 (br s, 1H), 8.64 (s, 1H), 8.46 (s, 1H), 8.19 (s, 1H), 8.12 (s, 1H), 8.09 (d, J = 8.8 Hz, 1H), 8.04 (d, J = 2.0 Hz, 1H), 8.01 (d, J = 1.8 Hz, 1H), 7.85 (q, J = 1.3 Hz, 1H), 7.83 (br s, 1H), 7.75 (q, J = 1.1 Hz, 1H), 7.43 (br d, J = 8.3 Hz, 1H), 7.32 (dd, J = 9.0, 2.0 Hz, 1H), 6.41 - 6.51 (m, 2 H), 6.27 (dd, J = 16.9, 2.0 Hz, 1H), 6.26 (dd, J = 17.0, 1.9 Hz, 1H), 5.75 (dd, J = 9.9, 1.8 Hz, 1H), 5.74 (dd, J = 10.1, 1.8 Hz, 1H), 4.05 (s, 3H), 3.92 (s, 3H), 2.28 (d, J = 1.3 Hz, 3H), 2.24 (d, J = 1.3 Hz, 3H) |
| Example 71 N-[1-[5-chloro-2-[(1-methylpyrazol- 4-yl)amino]pyrimidin-4-yl]-3,7- dimethyl-indol-5-yl]prop-2-enamide | 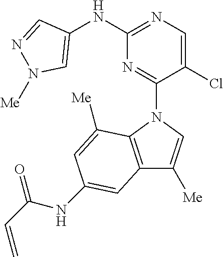 [SMILES: C=CC(=O)Nc1cc(C)c2c(c1)c(C)cn2-c1nc(Nc2cnn(C)c2)ncc1Cl] | LCMS m/z = 422 (M + 1); ¹H NMR (600 MHz, DMSO-d6) δ: 10.10 (1H, s), 9.85 -10.00 (1H, m), 8.64 (1H, br s), 7.85 - 7.95 (2H, m), 7.45 (1H, br s), 7.36 (1H, s), 7.23 (1H, s), 6.47 (1H, dd, J = 17.0, 10.2 Hz), 6.26 (1H, dd, J = 16.9, 2.0 Hz), 5.72 - 5.76 (1H, m), 3.61- 3.83 (3H, m), 2.26 (3H, s), 2.11 (3H, br s) |
| Example 72 N-[1-[5-chloro-2-[(3-methyltriazol-4- yl)amino]pyrimidin-4-yl]-3-methyl- indol-5-yl]prop-2-enamide | 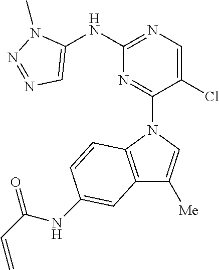 [SMILES: C=CC(=O)Nc1ccc2c(c1)c(C)cn2-c1nc(Nc2cnnn2C)ncc1Cl] | LCMS m/z = 409, 411 (M + 1, 1 Cl isotopic distribution); ¹H NMR (DMSO-d6, 500 MHz) δ 10.64 (s, 1H), 10.18 (s, 1H), 8.69 (s, 1H), 8.06 (d, J = 1.81 Hz, 1H), 7.91 (br s, 1H), 7.83 (s, 1H), 7.73 (s, 1H), 7.44 (dd, J = 1.73, 8.92 Hz, 1H), 6.47 (dd, J = 10.00, 17.04 Hz, 1H), 6.27 (dd, J = 1.88, 16.85 Hz, 1H), 5.75 (dd, J = 1.85, 10.07 Hz, 1H), 4.07 (s, 3H), 2.29 (s, 3H) |
| Example 73 N-[1-[5-chloro-2-[3-(4- methylpiperazin-1-yl)anilino]- pyrimidin-4-yl]-3-methyl-indol-5- yl]prop-2-enamide | 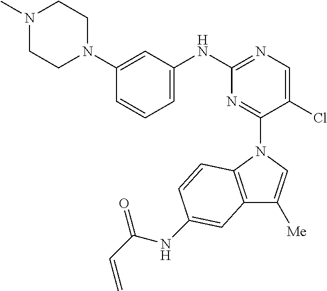 [SMILES: C=CC(=O)Nc1ccc2c(c1)c(C)cn2-c1nc(Nc2cccc(N3CCN(C)CC3)c2)ncc1Cl] | LCMS m/z = 502.2, 504.2 (M + H), 1 Cl isotopic distribution); ¹H NMR (DMSO-d6, 500 MHz) δ: 10.19 (s, 1H), 9.83 (s, 1H), 8.67 (s, 1H), 8.08 (d, 1H, J = 1.8 Hz), 7.82 (br d, 1H, J = 8.5 Hz), 7.69 (s, 1H), 7.47 (br s, 1H), 7.42 (dd, 1H, J = 1.8, 8.9 Hz), 7.07-7.10 (m, 2H, J = 6.1 Hz), 6.45-6.56 (m, 1H), 6.48 (dd, 1H, J = 10.2, 16.9 Hz), 6.27 (dd, 1H, J = 2.0, 16.9 Hz), 5.73- 5.76 (m, 1H), 2.97 (br s, 4H), 2.32 (br s, 4H), 2.29 (d, 3H, J = 1.2 Hz), 2.15 (s, 3H) |
| Example 74 N-[1-[5-chloro-2-(4- morpholinoanilino)pyrimidin-4-yl]- 3,7-dimethyl-indol-5-yl]prop-2- enamide | 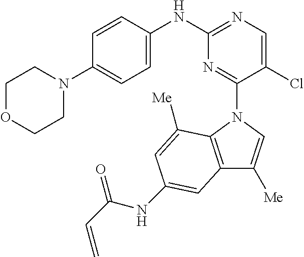 [SMILES: C=CC(=O)Nc1cc(C)c2c(c1)c(C)cn2-c1nc(Nc2ccc(N3CCOCC3)cc2)ncc1Cl] | LCMS m/z = 503.21 (M + H) (1 Cl isotopic distribution); ¹H NMR (600 MHz, DMSO-d6) δ = 10.09 (s, 1H), 9.82 (s, 1H), 8.65 (s, 1H), 7.90 (d, J = 1.7 Hz, 1H), 7.51; (br d, J = 8.8 Hz, 2H), 7.34 (d, J = 1.1 Hz, 1H), 7.20 (s, 1H), 6.85 (br d, J = 8.4 Hz, 2H), 6.47 (dd, J = 17.0, 10.2 Hz, 1H), 6.25 (dd, J = 17.0, 1.9 Hz, 1H), 5.72 - 5.75 (m, 1H), 3.68 - 3.74 (m, 4H), 2.99 - 3.04 (m, 4H), 2.25 (d, J = 1.3 Hz, 3H), 2.12 (s, 3H) |
| Example 75 N-[1-[5-chloro-2-[4- (morpholinomethyl)anilino]pyrimidin- 4-yl]-3-methyl-indol-5-yl]prop-2- enamide | 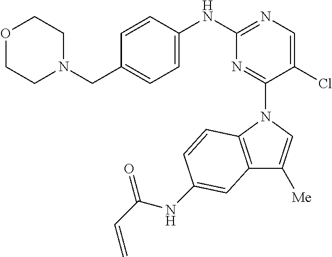 [SMILES: C=CC(=O)Nc1ccc2c(c1)c(C)cn2-c1nc(Nc2ccc(CN3CCOCC3)cc2)ncc1Cl] | LCMS m/z = 503, 505 (M + 1) (1Cl isotopic distribution); ¹H NMR (500 MHz, DMSO-d6) δ: 10.18 (s, 1H), 9.95 (s, 1H), 8.66 (s, 1H), 8.05 (d, J = 1.8 Hz, 1H), 7.87 (br d, J = 9.2 Hz, 1H), 7.73 - 7.69 (m, 1H), 7.66 (d, J = 8.2 Hz, 2H), 7.43 (dd, J = 8.9, 1.8 Hz, 1H), 7.20 (d, J = 8.5 Hz, 2H), 6.48 (dd, J = 16.9, 10.2 Hz, 1H), 6.27 (dd, J = 16.9, 2.0 Hz, 1H), 5.75 (dd, J = 10.1, 2.1 Hz, 1H), 3.55 (br t, J = 4.4 Hz, 4H), 3.41 - 3.37 (m, 2H), 2.32 (br s, 4H), 2.29 (d, J = 0.9 Hz, 3H) |
| Example 76 N-[1-[5-fluoro-2-[(2-methylpyrazol-3- yl)amino]pyrimidin-4-yl]-3-methyl- indazol-5-yl]prop-2-enamide | 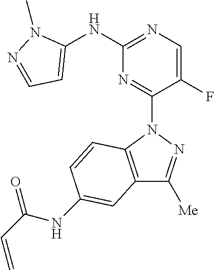 [SMILES: C=CC(=O)Nc1ccc2c(c1)c(C)nn2-c1nc(Nc2ccnn2C)ncc1F] | LCMS m/z = 393 (M + 1); ¹H NMR (DMSO-d6, 500 MHz): δ 10.37 (br s, 1H), 9.48 (br s, 1H), 8.55 (d, J = 4.9 Hz, 1H), 8.32 (d, J = 1.5 Hz, 1H), 8.07 (br s, 1H), 7.52 (br d, J = 8.9 Hz, 1H), 7.45 (br s, 1H), 6.46 (dd, J = 17.1, 10.7 Hz, 1H), 6.30 (dd, J = 17.4, 1.8 Hz, 1H), 6.23 (d, J = 2.1 Hz, 1H), 5.79 (dd, J = 11.0, 2.7 Hz, 1H), 3.65 (s, 3H), 2.55 (s, 3H) |
| Example 77 N-[3-methyl-1-[5-methyl-2-[(2- methylpyrazol-3-yl)amino]pyrimidin- 4-yl]indazol-5-yl]prop-2-enamide | 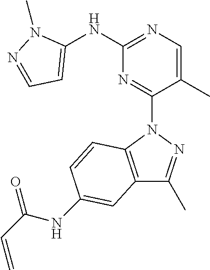 [SMILES: C=CC(=O)Nc1ccc2c(c1)c(C)nn2-c1nc(Nc2ccnn2C)ncc1C] | LCMS m/z = 389 (M + 1); ¹H NMR (500 MHz, DMSO-d6) δ: 10.33 (s, 1H), 9.27 (s, 1H), 8.35 (s, 1H), 8.31 (d, J = 1.8 Hz, 1H), 8.16 - 7.89 (m, 1H), 7.50 - 7.36 (m, 2H), 6.46 (dd, J = 17.1, 10.1 Hz, 1H), 6.29 (dd, J = 16.9, 2.0 Hz, 1H), 6.22 (d, J = 1.8 Hz, 1H), 5.80 - 5.75 (m, 1H), 3.64 (s, 3H), 2.56 - 2.52 (m, 3H), 2.51 (br s, 3H) |
| Example 78 N-[1-[5-chloro-2-[3- (morpholinomethyl)anilino]pyrimidin- 4-yl]-3-methyl-indol-5-yl]prop-2- enamide | 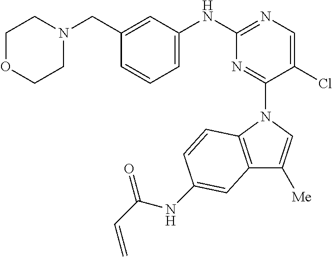 [SMILES: C=CC(=O)Nc1ccc2c(c1)c(C)cn2-c1nc(Nc2cccc(CN3CCOCC3)c2)ncc1Cl] | LCMS m/z = 503 (MH+, 1 Cl isotopic distribution); ¹H NMR (500 MHz, DMSO-d6) δ = 10.18 (s, 1H), 9.94 (s, 1H), 8.67 (s, 1H), 8.05 (d, J = 1.83 Hz, 1H), 7.87 (br d, J = 8.54 Hz, 1H), 7.72 (d, J = 0.92 Hz, 1H), 7.67 (s, 1H), 7.63 (br d, J = 8.24 Hz, 1H), 7.44 (dd, J = 8.85, J = 1.83 Hz, 1H), 7.22 (t, J = 7.78 Hz, 1H), 6.94 (d, J = 7.63 Hz, 1H), 6.47 (dd, J = 16.94, J = 10.22 Hz, 1H), 6.27 (dd, J = 16.94, J = 1.98 Hz, 1H), 5.75 (dd, J = 10.22, J = 1.98 Hz, 1H), 3.52 - 3.55 (m, 1H), 3.51 (br t, J = 4.27 Hz, 3H), 3.39 (s, 2H), 2.29 (br s, 7H) |
| Example 79 N-[3-methyl-1-[2-[(1-methylpyrazol- 4-yl)amino]pyrimidin-4-yl]indol-5- yl]prop-2-enamide | 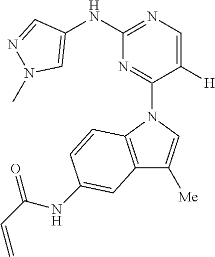 [SMILES: C=CC(=O)Nc1ccc2c(c1)c(C)cn2-c1ccnc(Nc2cnn(C)c2)n1] | LCMS m/z = 374 (M + 1); ¹H NMR (DMSO-d6, 500 MHz) δ: 9.88 (s, 1H), 9.11 (s, 1H), 8.5-8.6 (m, 1H), 8.37 (d, 1H, J = 5.5 Hz), 7.99 (d, 1H, J = 1.5 Hz), 7.86 (s, 1H), 7.80 (s, 1H), 7.54 (s, 1H), 7.49 (dd, 1H, J = 2.0, 9.0 Hz), 6.94 (d, 1H, J = 5.8 Hz), 6.48 (dd, 1H, J = 10.4, 17.1 Hz), 6.28 (dd, 1H, J = 1.8, 17.1 Hz), 5.72 (dd, 1H, J = 1.8, 10.1 Hz), 3.84 (s, 3H), 2.30 (d, 3H, J = 0.9 Hz) |
| Example 80 N-[1-[5-fluoro-2-[(2-methylpyrazol-3- yl)amino]pyrimidin-4-yl]-3-methyl- pyrrolo[2,3-b]pyridin-5-yl]prop-2- enamide | 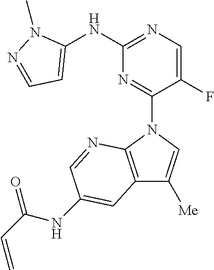 [SMILES: C=CC(=O)Nc1cnc2c(c1)c(C)cn2-c1nc(Nc2ccnn2C)ncc1F] | LCMS m/z = 393 (M + 1); ¹H NMR (400 MHz, DMSO-d6) δ: 10.38 (s, 1H), 9.68 (s, 1H), 8.69 (d, J = 2.7 Hz, 1H), 8.46 (d, J = 2.2 Hz, 1H), 8.43 (d, J = 2.1 Hz, 1H), 7.61 (s, 1H), 7.32 (d, J = 2.0 Hz, 1H), 6.46 - 6.42 (m, 1H), 6.33 - 6.26 (m, 2H), 5.81 - 5.77 (m, 1H), 3.70 (s, 3H), 2.30 (d, J = 1.1 Hz, 3H) |
| Example 81 N-[3-methyl-1-[5-methyl-2-[(1- methylpyrazol-3-yl)amino]pyrimidin- 4-yl]indol-5-yl]prop-2-enamide | 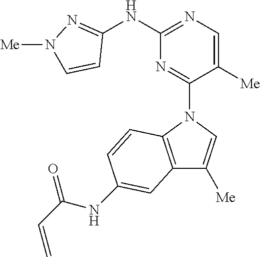 [SMILES: C=CC(=O)Nc1ccc2c(c1)c(C)cn2-c1nc(Nc2ccn(C)n2)ncc1C] | LCMS m/z = 388 (M + 1); ¹H NMR (300 MHz, DMSO-d6) δ: 10.12 (s, 1H), 9.71 (s, 1H), 8.41 (s, 1H), 8.03 (s, 1H), 7.83 (d, J = 8.7 Hz, 1H), 7.55 (s, 1H), 7.49 (d, J = 1.9 Hz, 1H), 7.37 (d, J = 8.9 Hz, 1H), 6.41-6.52 (m, 2H), 6.25 (dd, J = 16.9, 2.3 Hz, 1H), 5.73 (dd, J = 10.1, 2.1 Hz, 1H), 3.73 (s, 3H), 2.28 (s, 3H), 2.21 (s, 3H) |
| Example 82 N-[3-methyl-1-[2-[(2-methylpyrazol- 3-yl)amino]pyrimidin-4-yl]indol-5- yl]prop-2-enamide | 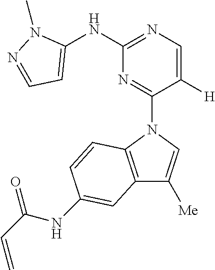 [SMILES: C=CC(=O)Nc1ccc2c(c1)c(C)cn2-c1ccnc(Nc2ccnn2C)n1] | LCMS m/z = 374 (M + 1); ¹H NMR (600 MHz, DMSO-d6) δ = 10.16 (1H, s), 9.37 (1H, s), 8.38 (1H, d, J = 5.7 Hz), 8.23 (1H, br s), 8.04 (1H, s), 7.90 (1H, s), 7.46 (1H, s), 7.24 - 7.38 (1H, m), 7.12 (1H, d, J = 5.9 Hz), 6.46 (1H, dd, J = 17.0 Hz, J = 10.2 Hz), 6.27 (1H, d, J = 16.9 Hz), 6.23 (1H, s), 5.75 (1H, d, J = 10.3 Hz), 3.67 (3H, s), 2.26 (3H, s) |
| Example 83 N-[3-methyl-1-[2-[(2-methylpyrazol- 3-yl)amino]pyrimidin-4- yl]pyrrolo[2,3-b]pyridin-5-yl]prop-2- enamide | 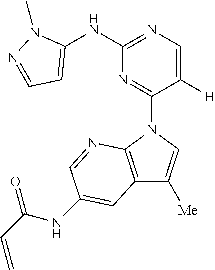 [SMILES: C=CC(=O)Nc1cnc2c(c1)c(C)cn2-c1ccnc(Nc2ccnn2C)n1] | LCMS m/z = 375 (M + 1); ¹H NMR (DMSO-d6, 600 MHz) δ 10.45 (s, 1H) 9.49 (br s, 1H) 8.56 (d, J = 2.3 Hz, 1H) 8.51 (d, J = 5.6 Hz, 1H) 8.48 (d, J = 2.3 Hz, 1H) 8.35 (d, J = 5.6 Hz, 1H) 8.02 (s, 1H) 7.40 (d, J = 1.8 Hz, 1H) 6.46 - 6.51 (m, 1H) 6.28 - 6.33 (m, 2H) 5.80 - 5.83 (m, 1H) 3.72 (s, 3H) 2.29 (d, J = 1.28 Hz, 3H) |
| Example 84 N-[1-[5-chloro-2-[(1-methylpyrazol- 4-yl)amino]pyrimidin-4-yl]-3,7- dimethyl-indol-5-yl]prop-2-enamide | 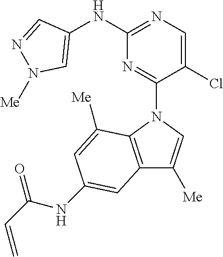 [SMILES: C=CC(=O)Nc1cc(C)c2c(c1)c(C)cn2-c1nc(Nc2cnn(C)c2)ncc1Cl] | LCMS m/z = 421.78 (MH+ 1 Cl isotopic distribution); ¹H NMR (600 MHz, DMSO-d6) δ = 10.10 (1H, s), 9.85 - 10.00 (1H, m), 8.64 (1H, br s), 7.85 - 7.95 (2H, m), 7.45 (1H, br s), 7.36 (1H, s), 7.23 (1H, s), 6.47 (1H, dd, J = 17.0 Hz, J = 10.2 Hz), 6.26 (1H, dd, J = 16.9 Hz, J = 2.0 Hz), 5.72 - 5.76 (1H, m), 3.61 - 3.83 (3H, m), 2.26 (3H, s), 2.11 (3H, br s) |
| Example 85 N-[3-methyl-1-[5-methyl-2-[(2- methylpyrazol-3-yl)amino]pyrimidin- 4-yl]pyrrolo[2,3-b]pyridin-5-yl]prop- 2-enamide | 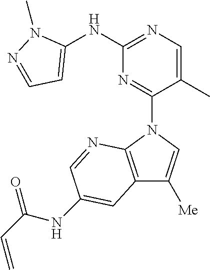 [SMILES: C=CC(=O)Nc1cnc2c(c1)c(C)cn2-c1nc(Nc2ccnn2C)ncc1C] | LCMS m/z = 389 (M + 1); ¹H NMR (DMSO-d6, 500 MHz) δ 10.34 (s, 1H), 9.49 (s, 1H), 8.51 (s, 1H), 8.45 (d, J = 2.20 Hz, 1H), 8.40 (d, J = 2.20 Hz, 1H), 7.58 (s, 1H), 7.31 (d, J = 1.83 Hz, 1H), 6.47 (dd, J = 10.18, 16.91 Hz, 1H), 6.29 (dd, J = 1.90, 17.04 Hz, 1H), 6.25 (d, J = 1.90 Hz, 1H), 5.79 (dd, J = 1.83, 10.11 Hz, 1H), 3.69 (s, 3H), 2.31 (s, 3H), 2.11 (s, 3H) |
| Example 86 N-[1-[5-chloro-2-[3-fluoro-4-[(4- methylpiperazin-1-yl)methyl]- anilino]pyrimidin-4-yl]-3-methyl- indol-5-yl]prop-2-enamide | 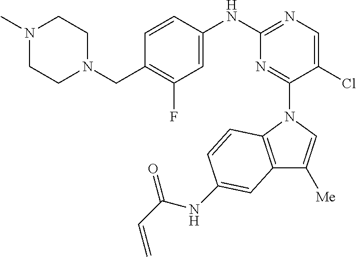 [SMILES: C=CC(=O)Nc1ccc2c(c1)c(C)cn2-c1nc(Nc2ccc(CN3CCN(C)CC3)c(F)c2)ncc1Cl] | LCMS m/z = 534.21, 536.21 (M + 1) (1 Cl isotopic distribution); ¹H NMR (500 MHz, DMSO-d6, 353K) δ: 9.92 (br s, 1H), 9.88 (s, 1H), 8.66 (s, 1H), 8.00 (s, 1H), 7.86 (d, J = 8.9 Hz, 1H), 7.70 - 7.63 (m, 2H), 7.48 - 7.42 (m, 2H), 7.26 (t, J = 8.4 Hz, 1H), 6.48 (dd, J = 16.9, 10.2 Hz, 1H), 6.31 - 6.26 (m, 1H), 5.74 - 5.71 (m, 1H), 3.46 (s, 2H), 2.43 - 2.39 (m, 4H), 2.34 - 2.30 (m, 7H), 2.16 (s, 3H) |
| Example 87 N-[3-methyl-1-[2-[(2-methylpyrazol- 3-yl)amino]pyrimidin-4-yl]indazol-5- yl]prop-2-enamide | 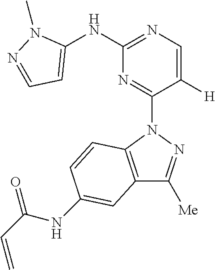 [SMILES: C=CC(=O)Nc1ccc2c(c1)c(C)nn2-c1ccnc(Nc2ccnn2C)n1] | LCMS m/z = 375 (M + 1); ¹H NMR (DMSO-d6, 500 MHz δ10.34 (s, 1H), 9.46 (s, 1H), 8.42 (d, J = 5.51 Hz, 1H), 8.30 (s, 1H), 8.23 (br s, 1H), 7.57-7.51 (m, 1H), 7.48 (s, 1H), 7.25 (d, 1H), 6.46 (dd, J = 10.17, 16.94 Hz, 1H), 6.33-6.23 (m, 2H), 5.81-5.77 (m, 1H), 3.67 (s, 3H), 2.55 (s, 3H) |
| Example 88 N-[1-[5-chloro-2-[3-[4-(2- hydroxyethyl)piperazin-1- yl]anilino]pyrimidin-4-yl]-3-methyl- indol-5-yl]prop-2-enamide | 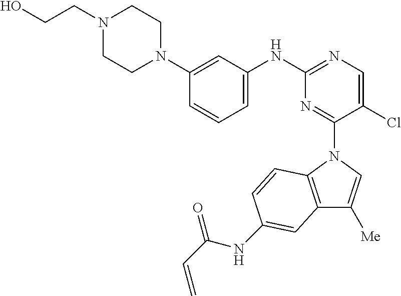 [SMILES: C=CC(=O)Nc1ccc2c(c1)c(C)cn2-c1nc(Nc2cccc(N3CCN(CCO)CC3)c2)ncc1Cl] | LCMS m/z = 533 (M + 1); (DMSO-d6, 300 MHz, δ: 10.18 (s, 1H) 9.82 (s, 1H) 8.66 (s, 1H) 8.07 (s, 1H) 7.80 (d, J = 8.9 Hz, 1H) 7.68 (s, 1H) 7.41 - 7.43 (m, 1H) 7.40 (s, 1H) 7.07 (br d, J = 4.7 Hz, 2H) 6.42 - 6.55 (m, 2H) 6.22 - 6.30 (m, 1H) 5.74 (br d, J = 11.8 Hz, 1H) 4.37 (br s, 1H) 3.48 (br dd, J = 4.7, 1.4 Hz, 2H) 2.91 - 3.03 (m, 4H) 2.38 - 2.45 (m, 4H), 2.35 (t, J = 6.3 Hz, 2H), 2.28 (s, 3H) |
| Example 89 N-[1-[5-chloro-2-[[1-(2- hydroxyethyl)pyrazol-4- yl]amino]pyrimidin-4-yl]-3-methyl- indol-5-yl]prop-2-enamide | 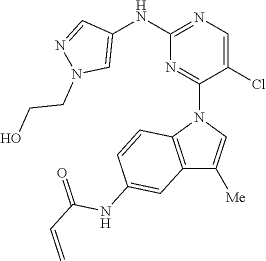 [SMILES: C=CC(=O)Nc1ccc2c(c1)c(C)cn2-c1nc(Nc2cnn(CCO)c2)ncc1Cl] | LCMS m/z = 438, 440 (M + 1) 1 Cl isotopic distribution); ¹H NMR (DMSO-d6, 500 MHz) δ: 9.88 (s, 1H), 9.51 (s, 1H), 8.55 (s, 1H), 7.99 (d, 1H, J = 1.5 Hz), 7.84 (s, 1H), 7.82 (d, 1H, J = 9.2 Hz), 7.66 (s, 1H), 7.56 (s, 1H), 7.46 (dd, 1H, J = 1.8, 8.9 Hz), 6.48 (dd, 1H, J = 10.2, 16.9 Hz), 6.28 (dd, 1H, J = 1.8, 17.1 Hz), 5.72 (dd, 1H, J = 2.0, 10.2 Hz), 4.55 (br s, 1H), 4.09 (t, 2H, J = 5.8 Hz), 3.74 (br t, 2H, J = 5.3 Hz), 2.31 (d, 3H, J = 0.9 Hz) |
| Example 90 N-[1-[5-fluoro-2-[[1-(2- hydroxyethyl)pyrazol-4- yl]amino]pyrimidin-4-yl]-3-methyl- indol-5-yl]prop-2-enamide | 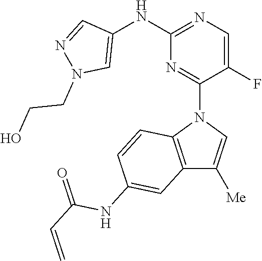 [SMILES: C=CC(=O)Nc1ccc2c(c1)c(C)cn2-c1nc(Nc2cnn(CCO)c2)ncc1F] | LCMS m/z = 422 (M + 1); ¹H NMR (DMSO-d6, 500 MHz) δ 9.94 (s, 1H), 9.27 (s, 1H), 8.49 (d, J = 4.55 Hz, 1H), 8.31 (d, J = 8.72 Hz, 1H), 8.02 (s, 1H), 7.88 (s, 1H), 7.65-7.63 (m, 1H), 7.55 (s, 1H), 7.49 (dd, J = 1.77, 9.03 Hz, 1H), 6.48 (dd, J = 10.18, 16.97 Hz, 1H), 6.28 (dd, J = 1.77, 16.90 Hz, 1H), 5.73 (dd, J = 1.77, 10.18 Hz, 1H), 4.62-4.59 (m, 1H), 4.12 (t, J = 5.71 Hz, 2H), 3.77 (quart, J = 5.56 Hz, 2H), 2.30 (s, 3H). |
| Example 91 N-[1-[5-chloro-2-[(6-morpholino-3- pyridyl)amino]pyrimidin-4-yl]-3- methyl-indol-5-yl]prop-2-enamide | 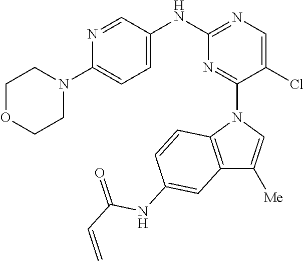 [SMILES: C=CC(=O)Nc1ccc2c(c1)c(C)cn2-c1nc(Nc2ccc(N3CCOCC3)nc2)ncc1Cl] | LCMS m/z = 490 (M + 1); ¹H NMR (500 MHz, DMdSO-d6) δ 10.16 (br.s, 1H), 9.74 (br.s, 1H), 8.60 (s, 1H), 8.40 (d, J = 2.5 Hz, 1H), 8.04 (d, J = 1.6 Hz, 1H), 7.86 (dd, J = 9.2, 2.5 Hz, 1H), 7.90- 7.73 (m, 1H), 7.69 (s, 1H), 7.41 (d, J = 8.6 Hz, 1H), 6.81 (d, J = 8.7 Hz, 1H), 6.52-6.43 (m, 1H), 6.26 (dd, J = 16.7, 1.8 Hz, 1H), 5.74 (dd, J = 10.1, 1.7 Hz, 1H), 3.69 (t, J = 4.5 Hz, 4H), 3.35 (t, J = 4.7 Hz, 4H), 2.27 (s, 3H) |
| Example 92 N-[1-methyl-3-[2-[(1-methylpyrazol- 3-yl)amino]pyrimidin-4-yl]indol-6- yl]prop-2-enamide | 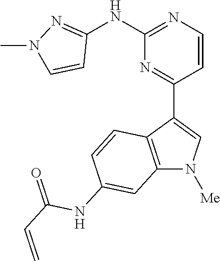 [SMILES: C=CC(=O)Nc1ccc2c(-c3ccnc(Nc4ccn(C)n4)n3)cn(C)c2c1] | LCMS m/z = 374 (M + 1); ¹H NMR (DMSO- d6, 300 MHz) δ 10.20 (s, 1H) 9.48 (s, 1H) 8.62 (d, J = 8.5 Hz, 1H) 8.23 - 8.29 (m, 1H) 8.21 (s, 1H) 8.10 (s, 1H) 7.55 (d, J = 1.9 Hz, 1H) 7.25 (br d, J = 7.1 Hz, 1H) 7.09 (d, J = 5.2 Hz, 1H) 6.57 (d, J = 2.0 Hz, 1H) 6.48 (dd, J = 17.16, 10.0 Hz, 1H) 6.26 (dd, J = 16.9, 2.0 Hz, 1H) 5.75 (dd, J = 9.7, 2.0 Hz, 1H) 3.81 (s, 3H) 3.77 (s, 3H) |
| Example 93 N-[1-[2-[(1-ethylpyrazol-3-yl)amino]- 5-methyl-pyrimidin-4-yl]-3- methylindol-5-yl]prop-2-enamide | 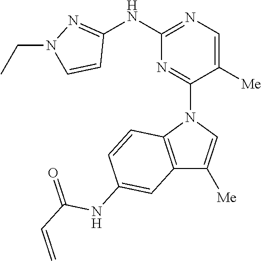 [SMILES: C=CC(=O)Nc1ccc2c(c1)c(C)cn2-c1nc(Nc2ccn(CC)n2)ncc1C] | LCMS m/z = 402 (M + 1); ¹H NMR (DMSO- d6, 300 MHz) δ 10.13 (s, 1H) 9.73 (s, 1H) 8.42 (s, 1H) 8.04 (d, J = 1.8 Hz, 1H) 7.84 (d, J = 8.8 Hz, 1H) 7.55 (d, J = 7.8 Hz, 2H) 7.38 (dd, J = 8.8, 1.8 Hz, 1H) 6.44 - 6.52 (m, 2H) 6.26 (dd, J = 16.9, 1.9 Hz, 1H) 5.72 - 5.76 (m, 1H) 4.02 (q, J = 7.1 Hz, 2H)2.29 (s, 3H) 2.22 (s, 3H) 1.35 (t, J = 7.17 Hz, 3H) |
| Example 94 N-[3-[2-[(1-ethylpyrazol-3- yl)amino]pyrimidin-4-yl]-1-methyl- indol-6-yl]prop-2-enamide | 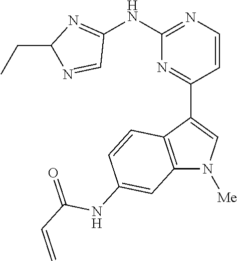 [SMILES: C=CC(=O)Nc1ccc2c(-c3ccnc(NC4=NC(CC)N=C4)n3)cn(C)c2c1] | LCMS m/z = 388 (M + 1); ¹H NMR (DMSO- d6, 300 MHz) δ 10.20 (s, 1H) 9.50 (s, 1H) 8.53 - 8.72 (m, 1H) 8.17 - 8.29 (m, 2H) 8.10 (s, 1H) 7.60 (s, 1H) 7.24 (br d, J = 8.3 Hz, 1H) 7.09 (br d, J = 4.8 Hz, 1H) 6.57 (br d, J = 1.7 Hz, 1H) 6.41 - 6.55 (m, 1H) 6.29 (s, 1H) 5.73 (br s, 1H) 4.05 (q, J = 7.2 Hz, 2H) 3.81 (s, 3H) 1.38 (br t, J = 7.1 Hz, 3H) |
| Example 95 N-[3-[2-[(1-ethylpyrazol-4- yl)amino]pyrimidin-4-yl]-1-methyl- indol-6-yl]prop-2-enamide | 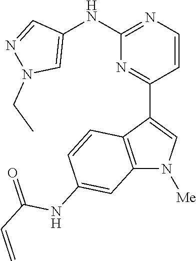 [SMILES: C=CC(=O)Nc1ccc2c(-c3ccnc(Nc4cnn(CC)c4)n3)cn(C)c2c1] | LCMS m/z = 388 (M + 1); ¹H NMR (500 MHz, DMSO-d6) δ 10.23 (s, 1H), 9.19 (br s, 1H), 8.77 - 8.30 (m, 1H), 8.27 (d, J = 5.2 Hz, 1H), 8.22 (s, 1H), 8.14 (s, 1H), 7.97 (s, 1H), 7.52 (s, 1H), 7.27 (br d, J = 8.5 Hz, 1H), 7.05 (d, J = 5.2 Hz, 1H), 6.49 (dd, J = 16.9, 10.2 Hz, 1H), 6.28 (dd, J = 16.8, 1.8 Hz, 1H), 5.83 - 5.69 (m, 1H), 4.11 (d, J = 7.3 Hz, 2H), 3.83 (s, 3H), 1.39 (br t, J = 7.2 Hz, 3H) |
| Example 96 N-[3-[2-[(1-isopropylpyrazol-4- yl)amino]pyrimidin-4-yl]-1-methyl- indol-6-yl]prop-2-enamide | 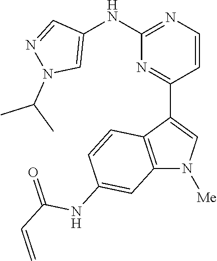 [SMILES: C=CC(=O)Nc1ccc2c(-c3ccnc(Nc4cnn(C(C)C)c4)n3)cn(C)c2c1] | LCMS m/z = 402 (M + 1); ¹H NMR δ: 10.23 (s, 1H), 9.21 - 9.12 (m, 1H), 8.87 - 8.31 (m, 1H), 8.27 (d, J = 5.5 Hz, 1H), 8.21 (s, 1H), 8.15 (s, 1H), 7.98 (s, 1H), 7.52 (s, 1H), 7.30 - 7.22 (m, 1H), 7.05 (d, J = 5.2 Hz, 1H), 6.49 (dd, J = 16.9, 10.2 Hz, 1H), 6.28 (dd, J = 17.1, 1.8 Hz, 1H), 5.79 - 5.73 (m, 1H), 4.47 (dt, J = 13.4, 6.6 Hz, 1H), 3.83 (s, 3H), 1.43 (d, J = 6.4 Hz, 6H) |
| Example 97 N-[3-methyl-1-[5-methyl-2-[(1- methylpyrazol-3-yl)amino]pyrimidin- 4-yl]pyrrolo[2,3-b]pyridin-5-yl]prop- 2-enamide | 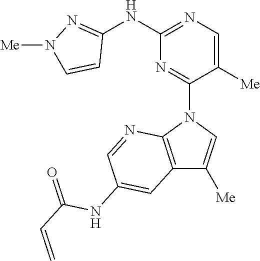 [SMILES: C=CC(=O)Nc1cnc2c(c1)c(C)cn2-c1nc(Nc2ccn(C)n2)ncc1C] | LCMS m/z = 389 (M + 1); ¹H NMR (DMSO- d6, 600 MHz) δ 10.34 (s, 1H) 9.76 (s, 1H) 8.45 (s, 2H) 8.39 (d, J = 2.2 Hz, 1H) 7.60 (s, 1H) 7.50 (d, J = 1.9 Hz, 1H) 6.55 (d, J = 2.2 Hz, 1H) 6.47 (dd, J = 16.7, 9.9 Hz, 1H) 6.28 (dd, J = 16.9, 1.9 Hz, 1H) 5.74 - 5.81 (m, 1H) 3.71 (s, 3H) 2.31 (s, 3H) 2.08 (s, 3H) |
| Example 98 N-[3-methyl-1-[2-[(1-methylpyrazol- 3-yl)amino]pyrimidin-4- yl]pyrrolo[2,3-b]pyridin-5-yl]prop-2- enamide | 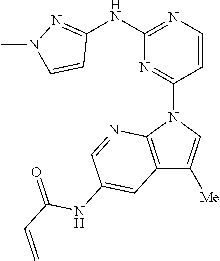 [SMILES: C=CC(=O)Nc1cnc2c(c1)c(C)cn2-c1ccnc(Nc2ccn(C)n2)n1] | LCMS m/z = 375 (M + 1); ¹H NMR (DMSO- d6, 600 MHz) δ 10.45 (s, 1H), 9.49 (s, 1H), 8.56 (d, J = 2.4 Hz, 1H), 8.51 (d, J = 5.5 Hz, 1H), 8.48 (d, J = 2.2 Hz, 1H), 8.36 (d, J = 5.7 Hz, 1H), 8.02 (s, 1H), 7.40 (d, J = 1.8 Hz, 1H), 6.48 (dd, J = 16.9, 10.1 Hz, 1H), 6.30 (s, 1H), 6.31 (dd, J = 18.0, 2.0 Hz, 1H), 5.82 (t, J = 5.0 Hz, 1H), 3.71 (s, 3H), 2.29 (d, J = 1.1 Hz, 3H) |
| Example 99 N-[1-[2-[(1-cyclopropylpyrazol-3- yl)amino]-5-methyl-pyrimidin-4-yl]- 3-methyl-indol-5-yl]prop-2-enamide | 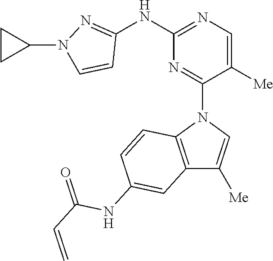 [SMILES: C=CC(=O)Nc1ccc2c(c1)c(C)cn2-c1nc(Nc2ccn(C3CC3)n2)ncc1C] | LCMS m/z = 414 (M + 1); ¹H NMR (500 MHz, DMSO-d6) δ: 10.13 (s, 1H), 9.75 (s, 1H), 8.42 (s, 1H), 8.04 (s, 1H), 7.82 (d, J = 8.5 Hz, 1H), 7.58 (d, J = 1.8 Hz, 1H), 7.56 (s, 1H), 7.38 (d, J = 8.9 Hz, 1H), 6.44-6.51 (m, 2H), 6.26 (d, J = 16.8 Hz, 1H), 5.74 (d, J = 10.4 Hz, 1H), 3.55-3.61 (m, 1H), 2.29 (s, 3H), 2.22 (s, 3H), 0.95-1.00 (m, 2H), 0.89-0.95 (m, 2H) |
| Example 100 N-[1-[2-[[1-(2-hydroxyethyl)pyrazol- 3-yl]amino]-5-methyl-pyrimidin-4- yl]-3-methyl-indol-5-yl]prop-2- enamide | 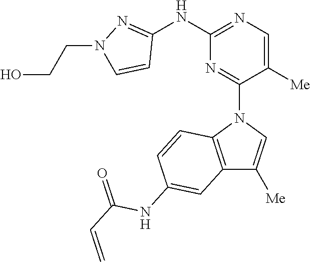 [SMILES: C=CC(=O)Nc1ccc2c(c1)c(C)cn2-c1nc(Nc2ccn(CCO)n2)ncc1C] | LCMS m/z = 418 (M + 1); ¹H NMR (500 MHz, DMSO-d6) δ: 10.13 (s, 1H), 9.76 (s, 1H), 8.42 (s, 1H), 8.04 (d, J = 1.5 Hz, 1H), 7.84 (d, J = 8.5 Hz, 1H), 7.56 (s, 1H), 7.52 (s, 1H), 7.37 (dd, J = 8.5, 1.5 Hz, 1H), 6.49 (s, 1H), 6.47 (dd, J = 17.0, 10.5 Hz, 1H), 6.26 (d, J = 17.5 Hz, 1H), 5.75 (d, J = 11.5 Hz, 1H), 4.86 (t, J = 5.5 Hz, 1H), 4.02 (t, J = 6 Hz, 2H), 3.71 (dd, J = 11.0, 5.5 Hz, 2H), 2.29 (s, 3H), 2.22 (s, 3H) |
| Example 101 N-[3-[2-[[1-(2-hydroxyethyl)pyrazol- 3-yl]amino]pyrimidin-4-yl]-1-methyl- indol-6-yl]prop-2-enamide | 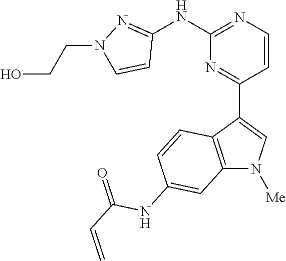 [SMILES: C=CC(=O)Nc1ccc2c(-c3ccnc(Nc4ccn(CCO)n4)n3)cn(C)c2c1] | LCMS m/z = 404 (M + 1); ¹H NMR (300 MHz, DMSO-d6) δ: 10.20 (s, 1H), 9.53 (s, 1H), 8.63 (d, J = 11.7 Hz, 1H), 8.24 (d, J = 12.3 Hz, 1H), 8.11 (s, 1H), 7.58 (d, J = 2.1, 1H), 7.24 (d, J = 8.7 Hz, 1H), 7.09 (d, J = 5.4 Hz, 1H), 6.59 (d, J = 2.1 Hz, 1H), 6.48 (dd, J = 17.1, 10.2 Hz, 1H), 6.26 (dd, J = 17.1, 2.1 Hz, 1H), 5.75 (dd, J = 10.1, 2.1 Hz, 1H), 4.88 (t, J = 5.4 Hz, 1H), 4.05 (t, J = 5.4 Hz, 2H), 3.81 (s, 3H), 3.74 (dd, J = 11.1 , 5.4 Hz, 2H) |
| Example 102 N-[1-[2-[(1,5-dimethylpyrazol-3- yl)amino]-5-methyl-pyrimidin-4-yl]- 3-methyl-indol-5-yl]prop-2-enamide | 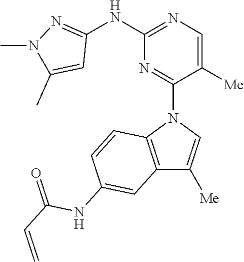 [SMILES: C=CC(=O)Nc1ccc2c(c1)c(C)cn2-c1nc(Nc2cc(C)n(C)n2)ncc1C] | LCMS m/z = 402 (M + 1); ¹H NMR (DMSO-d6, 300 MHz) δ: 10.13 (s, 1H), 9.57 (s, 1H), 8.38 (s, 1H), 8.04 (d, 1H, J = 1.6 Hz), 7.86 (d, 1H, J = 8.9 Hz), 7.55 (s, 1H), 7.3-7.4 (m, 1H), 6.44 (td, 1H, J = 10.1, 17.1 Hz), 6.32 (s, 1H), 6.25 (dd, 1H, J = 2.3, 17.1 Hz), 5.73 (dd, 1H, J = 2.0, 10.0 Hz), 3.61 (s, 3H), 2.27 (s, 3H), 2.20 (s, 3H), 2.17 (s, 3H) |
| Example 103 N-[3-[2-[(1,5-dimethylpyrazol-3- yl)amino]pyrimidin-4-yl]-1-methyl- indol-6-yl]prop-2-enamide | 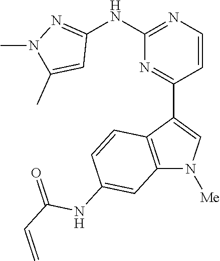 [SMILES: C=CC(=O)Nc1ccc2c(-c3ccnc(Nc4cc(C)n(C)n4)n3)cn(C)c2c1] | LCMS m/z = 388 (M + 1); ¹H NMR (DMSO-d6, 600 MHz) δ: 10.22 (s, 1H), 9.37 (s, 1H), 8.66 (br d, 1H, J = 8.3 Hz), 8.25 (d, 1H, J = 5.3 Hz), 8.21 (s, 1H), 8.13 (d, 1H, J = 1.7 Hz), 7.25 (dd, 1H, J = 1.7, 8.7 Hz), 7.08 (d, 1H, J = 5.5 Hz), 6.49 (dd, 1H, J = 10.1, 16.9 Hz), 6.43 (s, 1H), 6.28 (dd, 1H, J = 1.9, 17.0 Hz), 5.76 (dd, 1H, J = 2.0, 10.1 Hz), 3.82 (s, 3H), 3.66 (s, 3H), 2.27 (s, 3H) |
| Example 104 2-methyl-N-[3-methyl-1-[5-methyl-2- [(2-methylpyrazol-3- yl)amino]pyrimidin-4-yl]indol-5- yl]prop-2-enamide | 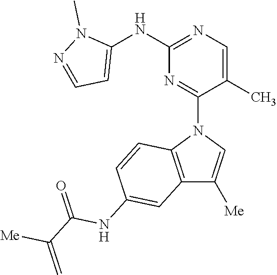 [SMILES: C=C(C)C(=O)Nc1ccc2c(c1)c(C)cn2-c1nc(Nc2ccnn2C)ncc1C] | LCMS m/z = 402 (M + 1); ¹H NMR δ: 9.74 (s, 1H), 9.36 (s, 1H), 8.42 (s, 1H), 7.96 (d, J = 1.4 Hz, 1H), 7.64 (d, J = 8.9 Hz, 1H), 7.55 (s, 1H), 7.37 (dd, J = 8.9, 1.7 Hz, 1H), 7.32 (d, J = 1.9 Hz, 1H), 6.22 (d, J = 1.7 Hz, 1H), 5.82 (s, 1H), 5.48 (s, 1H), 3.66 (s, 3H), 2.26 (s, 3H), 2.23 (s, 3H), 1.96 (s, 3H) |
| Example 105 N-[3-[2-[[1- (cyclopropylmethyl)pyrazol-4- yl]amino]pyrimidin-4-yl]-1-methyl- indol-6-yl]prop-2-enamide | 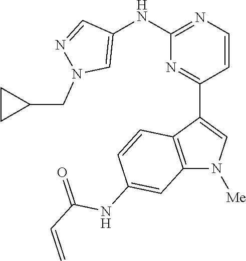 [SMILES: C=CC(=O)Nc1ccc2c(-c3ccnc(Nc4cnn(CC5CC5)c4)n3)cn(C)c2c1] | LCMS m/z = 414 (M + 1); ¹H NMR δ 10.23 (s, 1H), 9.20 (s, 1H), 8.55 (br s, 1H), 8.28 (d, J = 5.32 Hz, 1H), 8.21 (s, 1H), 8.14 (d, J = 1.68 Hz, 1H), 8.01 (s, 1H), 7.54-7.53 (m, 1H), 7.28 (d, J = 8.60 Hz, 1H), 7.06 (d, J = 5.29 Hz, 1H), 6.49 (dd, J = 10.12, 16.83 Hz, 1H), 6.28 (dd, J = 1.88, 16.93 Hz, 1H), 5.76 (dd, J = 1.88, 10.14 Hz, 1H), 3.95 (d, J = 7.18 Hz, 2H), 3.83 (s, 3H), 1.28-1.21 (m, 1H), 0.56-0.52 (m, 2H), 0.39-0.35 (m, 2H). |
| Example 106 N-[3-[2-[[1-(2-hydroxyethyl)pyrazol- 4-yl]amino]pyrimidin-4-yl]-1-methyl- indol-6-yl]prop-2-enamide | 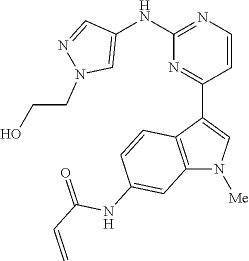 [SMILES: C=CC(=O)Nc1ccc2c(-c3ccnc(Nc4cnn(CCO)c4)n3)cn(C)c2c1] | LCMS m/z = 404 (M + 1); ¹H NMR δ 10.24 (s, 1H), 9.21 (s, 1H), 8.28 (d, J = 5.30 Hz, 1H), 8.23-8.21 (m, 2H), 8.15 (d, J = 1.35 Hz, 1H), 7.99 (s, 1H), 7.54 (s, 1H), 7.28 (dd, J = 1.54, 8.59 Hz, 1H), 7.06 (d, J = 5.36 Hz, 1H), 6.50 (dd, J = 10.15, 16.95 Hz, 1H), 6.28 (dd, J = 1.94, 16.88 Hz, 1H), 5.76 (dd, J = 1.91, 10.06 Hz, 1H), 4.90 (br s, 1H), 4.13-4.10 (m, 2H), 3.83 (s, 3H), 3.76- 3.73 (m, 2H). |
| Example 107 (E)-4-(dimethylamino)-N-[3-methyl- 1-[5-methyl-2-[(2-methylpyrazol-3- yl)amino]pyrimidin-4-yl]indol-5- yl]but-2-enamide | 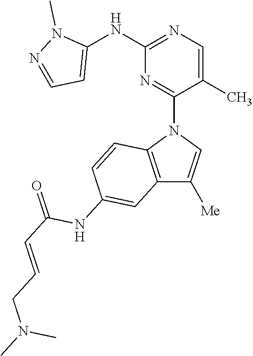 | LCMS m/z = 445 (M + 1); ¹H NMR (500 MHz, DMSO-d6) δ 10.04 (s, 1H), 9.36 (s, 1H), 8.43 (s, 1H), 8.03 (d, J = 1.5 Hz, 1H), 7.66 (br d, J = 8.9 Hz, 1H), 7.56 (s, 1H), 7.33 (d, J = 1.8 Hz, 1H), 7.30 (br d, J = 8.9 Hz, 1H), 6.72 (dt, J = 15.3, 5.9 Hz, 1H), 6.29 (d, J = 15.3 Hz, 1H), 6.22 (d, J = 1.8 Hz, 1H), 3.66 (s, 3H), 3.06 (d, J = 6.1 Hz, 2H), 2.27 (s, 3H), 2.24 (s, 3H), 2.18 (s, 6H) |
| Example 108 N-[3-methyl-1-[5-methyl-2-[(2- methylpyrazol-3-yl)amino]pyrimidin- 4-yl]indol-5-yl]prop-2-ynamide | 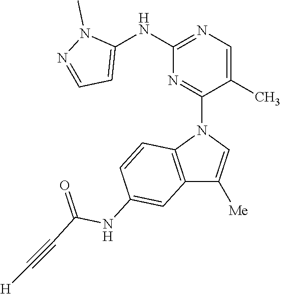 [SMILES: C#CC(=O)Nc1ccc2c(c1)c(C)cn2-c1nc(Nc2ccnn2C)ncc1C] | LCMS m/z = 386 (M + 1); ¹H NMR (500 MHz, DMSO-d6) δ: 10.76 (s, 1H), 9.38 (s, 1H), 8.44 (s, 1H), 7.93 (s, 1H), 7.66 (br d, J = 9.2 Hz, 1H), 7.58 (s, 1H), 7.33 (d, J = 1.5 Hz, 1H), 7.29 (br d, J = 8.9 Hz, 1H), 6.22 (d, J = 1.5 Hz, 1H), 4.37 (s, 1H), 3.66 (s, 3H), 2.26 (s, 3H), 2.23 (s, 3H) |
| Example 109 N-[3-methyl-1-[5-methyl-2-[(2- methylpyrazol-3-yl)amino]pyrimidin- 4-yl]indol-5-yl]ethenesulfonamide | 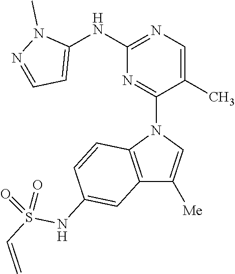 | LCMS m/z = 386 (M + 1); ¹H NMR (500 MHz, DMSO-d6) δ: 9.73 (s, 1H), 9.36 (s, 1H), 8.44 (s, 1H), 7.65 (br d, J = 8.9 Hz, 1H), 7.57 (s, 1H), 7.34 - 7.31 (m, 2H), 7.01 (br d, J = 8.9 Hz, 1H), 6.74 (dd, J = 16.5, 10.1 Hz, 1H), 6.21 (d, J = 1.5 Hz, 1H), 6.03 - 5.95 (m, 2H), 3.66 (s, 3H), 2.25 (s, 3H), 2.22 (s, 3H) |
| Example 110 2-fluoro-N-[3-methyl-1-[5-methyl-2- [(2-methylpyrazol-3- yl)amino]pyrimidin-4-yl]indol-5- yl]prop-2-enamide | 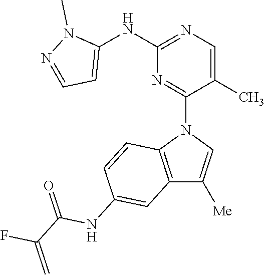 [SMILES: C=C(F)C(=O)Nc1ccc2c(c1)c(C)cn2-c1nc(Nc2ccnn2C)ncc1C] | LCMS m/z = 406 (M + 1); ¹H NMR (500 MHz, DMSO-d6) δ: 10.28 (s, 1H), 9.38 (s, 1H), 8.45 (s, 1H), 8.00 (d, J = 1.8 Hz, 1H), 7.68 (br d, J = 9.2 Hz, 1H), 7.58 (s, 1H), 7.44 (br d, J = 8.9 Hz, 1H), 7.33 (d, J = 1.5 Hz, 1H), 6.23 (d, J = 1.5 Hz, 1H), 5.77 - 5.61 (m, 1H), 5.41 (dd, J = 15.6, 3.4 Hz, 1H), 3.67 (s, 3H), 2.28 (s, 3H), 2.24 (s, 3H) |
| Example 111 N-[1-methyl-3-[2-[(5-methyl-1H- pyrazol-3-yl)amino]pyrimidin-4- yl]indol-6-yl]prop-2-enamide | 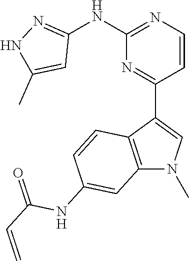 [SMILES: C=CC(=O)Nc1ccc2c(-c3ccnc(Nc4cc(C)[nH]n4)n3)cn(C)c2c1] | LCMS m/z = 374 (M + 1); ¹H NMR (DMSO- d6, 500 MHz) δ 10.27 (s, 1H) 8.59 (d, J = 8.5 Hz, 1H) 8.56 (d, J = 5.4 Hz, 1H) 8.39 (s, 1H) 8.16 (s, 1H) 7.57 (d, J = 5.4 Hz, 1H) 7.34 (dd, J = 8.5, 1.5 Hz, 1H) 6.71 (s, 2H) 6.50 (dd, J = 16.7, 10.0 Hz, 1H) 6.28 (dd, J = 16.7, 1.8 Hz, 1H) 5.75-5.80 (m, 1H) 5.30 (s, 1H) 3.86 (s, 3H) 2.14 (s, 3H) |
| Example 112 N-[3-[2-[(1-cyclopropylpyrazol-3- yl)amino]pyrimidin-4-yl]-1-methyl- indol-6-yl]prop-2-enamide | 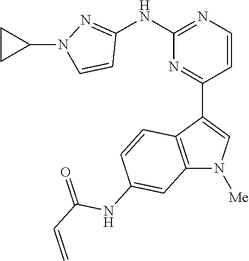 [SMILES: C=CC(=O)Nc1ccc2c(-c3ccnc(Nc4ccn(C5CC5)n4)n3)cn(C)c2c1] | LCMS m/z = 401 (M + 1); ¹H NMR (500 MHz, DMSO-d6) δ: 10.13 (s, 1H), 9.75 (s, 1H), 8.42 (s, 1H), 8.04 (s, 1H), 7.82 (d, J = 8.5 Hz, 1H), 7.58 (d, J = 1.8 Hz, 1H), 7.56 (s, 1H), 7.38 (d, J = 8.9 Hz, 1H), 6.44-6.51 (m, 2H), 6.26 (d, J = 16.8 Hz, 1H), 5.74 (d, J = 10.4 Hz, 1H), 3.55-3.61 (m, 1H), 2.29 (s, 3H), 2.22 (s, 3H), 0.95-1.00 (m, 2H), 0.89-0.95 (m, 2H) |
| Example 113 N-[3-methyl-1-[5-methyl-2-[[1- (oxetan-3-yl)pyrazol-3-yl]amino]- pyrimidin-4-yl]pyrrolo[2,3-b]pyridin- 5-yl]prop-2-enamide | 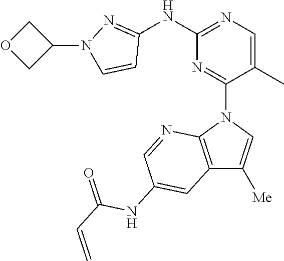 [SMILES: C=CC(=O)Nc1cnc2c(c1)c(C)cn2-c1nc(Nc2ccn(C3COC3)n2)ncc1C] | 1H NMR (DMSO-d6, 500 MHz) δ: 10.35 (s, 1H), 10.00 (s, 1H), 8.49 (s, 1H), 8.46 (d, 1H, J = 2.1 Hz), 8.41 (d, 1H, J = 2.1 Hz), 7.70 (d, 1H, J = 2.1 Hz), 7.62 (s, 1H), 6.68 (d, 1H, J = 2.4 Hz), 6.47 (dd, 1H, J = 10.2, 16.9 Hz), 6.29 (dd, 1H, J = 1.7, 16.9 Hz), 5.79 (dd, 1H, J = 1.7, 10.2 Hz), 5.46 (quin, 1H, J = 7.0 Hz), 4.86-4.89 (m, 4H), 2.32 (s, 3H), 2.10 (s, 3H) |
| Example 114 N-[1-[2-[(1-ethylpyrazol-3-yl)amino]- 5-methyl-pyrimidin-4-yl]-3-methyl- pyrrolo[2,3-b]pyridin-5-yl]prop-2- enamide | 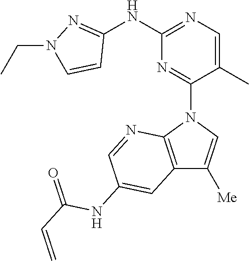 [SMILES: C=CC(=O)Nc1cnc2c(c1)c(C)cn2-c1nc(Nc2ccn(CC)n2)ncc1C] | LCMS m/z = 403 (M + 1); 1H NMR (500 MHz, DMdSO-d6): 10.34 (br.s, 1H), 9.77 (br.s, 1H), 8.46 (m, 2H), 8.40 (d, J = 2.1 Hz, 1H), 7.60 (s, 1H), 7.55 (d, J = 2.3 Hz, 1H), 6.56 (d, J = 2.2 Hz, 1H), 6.51-6.42 (m, 1H), 6.29 (dd, J = 16.6, 1.9 Hz, 1H), 5.78 (dd, J = 10.2, 1.7 Hz, 1H), 4.00 (q, J = 7.1 Hz, 2H), 2.32 (s, 3H), 2.09 (s, 3H), 1.33 (t, J = 7.1 Hz, 3H) |
| Example 115 (E)-4-(dimethylamino)-N-[1-[2-[(1- ethylpyrazol-3-yl)amino]-5-methyl- pyrimidin-4-yl]-3-methyl-pyrrolo[2,3- b]pyridin-5-yl]but-2-enamide | 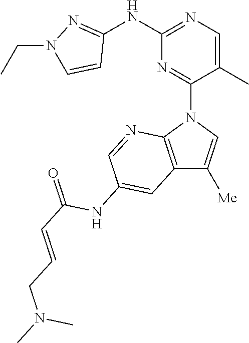 [SMILES: CCn1ccc(Nc2ncc(C)c(-n3cc(C)c4cc(NC(=O)/C=C/CN(C)C)cnc43)n2)n1] | LCMS m/z = 460 (M + 1); ¹H NMR (500 MHz, DMSO-d6): 10.25 (br.s, 1H), 9.76 (br.s, 1H), 8.50-8.42 (m, 2H), 8.37 (d, J = 1.9 Hz, 1H), 7.59 (s, 1H), 7.56 (d, J = 2.2 Hz, 1H), 6.80-6.73 (m, 1H), 6.56 (d, J = 2.2 Hz, 1H), 6.33-6.30 (m, 1H), 4.00 (q, J = 7.1 Hz, 2H), 3.07 (d, J = 5.7 Hz, 2H), 2.31 (s, 3H), 2.18 (s, 6H), 2.08 (s, 3H), 1.33 (t, J = 7.4 Hz, 3H) |
| Example 116 2-chloro-N-[3-methyl-1-[5-methyl-2- [(1-methylpyrazol-3- yl)amino]pyrimidin-4-yl]pyrrolo[2,3- b]pyridin-5-yl]prop-2-enamide | 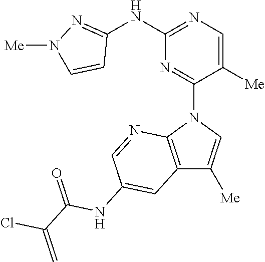 [SMILES: C=C(Cl)C(=O)Nc1cnc2c(c1)c(C)cn2-c1nc(Nc2ccn(C)n2)ncc1C] | LCMS m/z = 423 (M + 1); ¹H NMR (DMSO- d6, 600 MHz) δ: 10.39 (s, 1H) 9.78 (s, 1H) 8.47 (d, J = 0.7 Hz, 1H) 8.45 (d, J = 2.3 Hz, 1H) 8.34 (d, J = 2.2 Hz, 1H) 7.63 (d, J = 1.1 Hz, 1H) 7.51 (d, J = 2.2 Hz, 1H) 6.56 (d, J = 2.2 Hz, 1H) 6.50 (d, J = 2.5 Hz, 1H) 6.12 (d, J = 2.5 Hz, 1H) 3.72 (s, 3H) 2.33 (d, J = 1.2 Hz, 3H) 2.09 (s, 3H) |
| Example 117 2-fluoro-N-[3-methyl-1-[5-methyl-2- [(1-methylpyrazol-3- yl)amino]pyrimidin-4-yl]pyrrolo[2,3- b]pyridin-5-yl]prop-2-enamide | 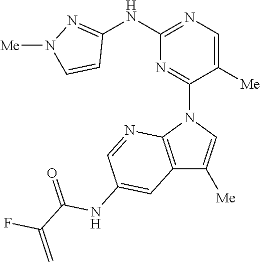 [SMILES: C=C(F)C(=O)Nc1cnc2c(c1)c(C)cn2-c1nc(Nc2ccn(C)n2)ncc1C] | LCMS m/z = 407 (M + 1); ¹H NMR (DMSO- d6, 600 MHz) δ: 10.51 (s, 1H) 9.78 (s, 1H) 8.50 (d, J = 2.2 Hz, 1H) 8.47 (s, 1H) 8.39 (d, J = 2.2 Hz, 1H) 7.63 (s, 1H) 7.51 (d, J = 2.2 Hz, 1H) 6.56 (d, J = 2.2 Hz, 1H) 5.64 - 5.85 (m, 1H) 5.46 (dd, J = 15.5, 3.6 Hz, 1H) 3.72 (s, 3H) 2.32 - 2.33 (m, 3H) 2.09 (s, 3H) |
| Example 118 2-methyl-N-[3-methyl-1-[5-methyl-2- [(1-methylpyrazol-3- yl)amino]pyrimidin-4-yl]pyrrolo[2,3- b]pyridin-5-yl]prop-2-enamide | 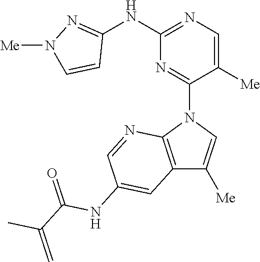 [SMILES: C=C(C)C(=O)Nc1cnc2c(c1)c(C)cn2-c1nc(Nc2ccn(C)n2)ncc1C] | LCMS m/z = 403 (M + 1); (DMSO- d6, 600 MHz) δ: 9.95 (s, 1H) 9.76 (s, 1H) 8.46 (d, J = 0.5 Hz, 1H) 8.45 (d, J = 2.3 Hz, 1H) 8.37 (d, J = 2.3 Hz, 1H) 7.60 (d, J = 1.1 Hz, 1H) 7.51 (d, J = 2.2 Hz, 1H) 6.57 (d, J = 2.2 Hz, 1H) 5.88 (s, 1H) 5.55 - 5.56 (m, 1H) 3.72 (s, 3H) 2.32 (d, J = 1.1 Hz, 3H) 2.09 (s, 3H) 1.98 (t, J = 1.1 Hz, 3H) |
| Example 119 N-[1-[2-[[1-(2-hydroxyethyl)pyrazol- 3-yl]amino]-5-methyl-pyrimidin-4- yl]-3-methyl-pyrrolo[2,3-b]pyridin-5- yl]prop-2-enamide | 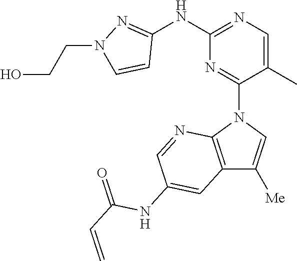 [SMILES: C=CC(=O)Nc1cnc2c(c1)c(C)cn2-c1nc(Nc2ccn(CCO)n2)ncc1C] | LCMS m/z = 419 (M + 1); 1H NMR (500 MHz, DMSO-d6) δ: 10.34 (s, 1H), 9.78 (s, 1H), 8.46 (s, 2H), 8.40 (d, J = 2.1 Hz, 1H), 7.61 (s, 1H), 7.53 (d, J = 2.1 Hz, 1H), 6.57 (d, J = 2.1 Hz, 1H), 6.47 (dd, J = 16.9, 10.2 Hz, 1H), 6.34 - 6.25 (m, 1H), 5.79 (dd, J = 10.1, 1.8 Hz, 1H), 4.85 (t, J = 5.3 Hz, 1H), 4.01 (t, J = 5.6 Hz, 2H), 3.69 (q, J = 5.6 Hz, 2H), 2.35 - 2.30 (m, 3H), 2.09 (s, 3H) |
| Example 120 N-[3-methyl-1-[5-methyl-2-[[1- (oxetan-3-yl)pyrazol-3- yl]amino]pyrimidin-4-yl]indol-5- yl]prop-2-enamide | 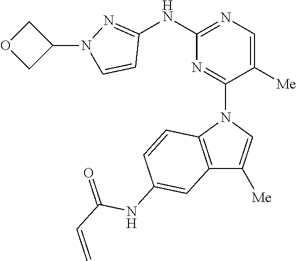 [SMILES: C=CC(=O)Nc1ccc2c(c1)c(C)cn2-c1nc(Nc2ccn(C3COC3)n2)ncc1C] | LCMS m/z = 430 (M + 1); 1H NMR (DMSO-d6, 500 MHz) δ: 10.14 (s, 1H), 9.97 (s, 1H), 8.44 (s, 1H), 8.05 (d, 1H, J = 1.8 Hz), 7.84 (d, 1H, J = 8.9 Hz), 7.69 (d, 1H, J = 2.4 Hz), 7.57 (s, 1H), 7.38 (dd, 1H, J = 1.8, 8.9 Hz), 6.61 (d, 1H, J = 2.1 Hz), 6.47 (dd, 1H, J = 10.2, 16.9 Hz), 6.26 (dd, 1H, J = 2.0, 16.9 Hz), 5.72-5.75 (m, 1H), 5.46 (quin, 1H, J = 7.0 Hz), 4.86-4.90 (m, 4H), 2.29 (s, 3H), 2.23 (s, 3H) |
| Example 121 N-[1-[2-[[1-(2-hydroxyethyl)pyrazol- 4-yl]amino]-5-methyl-pyrimidin-4- yl]-3-methyl-indol-5-yl]prop-2- enamide | 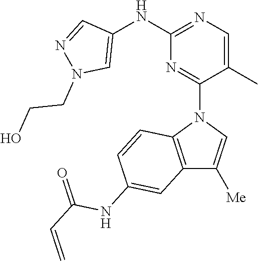 [SMILES: C=CC(=O)Nc1ccc2c(c1)c(C)cn2-c1nc(Nc2cnn(CCO)c2)ncc1C] | LCMS m/z = 418 (M + 1); 1H NMR (DMSO-d6, 500 MHz) δ: 10.16 (s, 1H), 9.46 (br s, 1H), 8.42 (s, 1H), 8.07 (s, 1H), 7.65-7.95 (m, 2H), 7.56 (s, 1H), 7.52 (s, 1H), 7.40 (br d, 1H, J = 8.5 Hz), 6.48 (dd, 1H, J = 10.2, 16.9 Hz), 6.26 (dd, 1H, J = 2.0, 16.9 Hz), 5.74 (dd, 1H, J = 1.8, 10.1 Hz), 4.04 (br s, 2H), 3.44 (bs, 1H + water), 3.68 (t, 3H, J = 1.0 Hz), 2.29 (s, 3H), 2.21 (s, 3H) |
| Example 122 (E)-4-(dimethylamino)-N-[1-[2-[[1- (2-hydroxyethyl)pyrazol-4-yl]amino]- 5-methyl-pyrimidin-4-yl]-3-methyl- indol-5-yl]but-2-enamide | 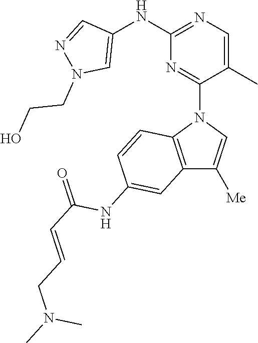 [SMILES: Cc1cnc(Nc2cnn(CCO)c2)nc1-n1cc(C)c2cc(NC(=O)/C=C/CN(C)C)ccc21] | LCMS m/z = 475 (M + 1); 1H NMR (DMSO-d6, 500 MHz) δ: 10.05 (s, 1H), 9.42 (br s, 1H), 8.42 (s, 1H), 8.07 (s, 1H), 7.70-7.90 (m, 2H), 7.55 (s, 1H), 7.52 (s, 1H), 7.37 (br d, 1H, J = 8.5 Hz), 6.72 (td, 1H, J = 6.1, 15.3 Hz), 6.30 (d, 1H, J = 15.3 Hz), 4.82 (br s, 1H), 4.04 (br s, 2H), 3.68 (br s, 2H), 3.07 (d, 2H, J = 5.8 Hz), 2.29 (s, 3H), 2.21 (s, 3H), 2.19 (s, 6H) |
| Example 123 N-[3-methyl-1-[5-methyl-2-[(5- methyl-1H-pyrazol-3- yl)amino]pyrimidin-4-yl]indol-5- yl]prop-2-enamide | 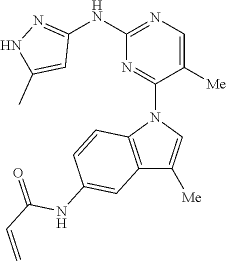 [SMILES: C=CC(=O)Nc1ccc2c(c1)c(C)cn2-c1nc(Nc2cc(C)[nH]n2)ncc1C] | LCMS m/z = 388 (M + 1); 1H NMR (500 MHz, DMSO-d6): 11.86 (br.s, 1H), 10.14 (br.s, 1H), 9.53 (br.s, 1H), 8.40 (s, 1H), 8.05 (d, J = 1.6 Hz, 1H), 7.84 (d, J = 8.9 Hz, 1H), 7.56 (s, 1H), 7.38 (dd, J = 8.8, 1.7 Hz, 1H), 6.52-6.43 (m, 1H), 6.38-6.22 (m, 2H), 5.73 (dd, J = 9.9, 1.9 Hz, 1H), 2.28 (s, 3H), 2.21 (s, 3H), 2.16 (s, 3H) |
| Example 124 tert-butyl 5-methyl-3-[[5-methyl-4-[3- methyl-5-(prop-2- enoylamino)pyrrolo[2,3-b]pyridin-1- yl]pyrimidin-2-yl]amino]pyrazole-1- carboxylate | 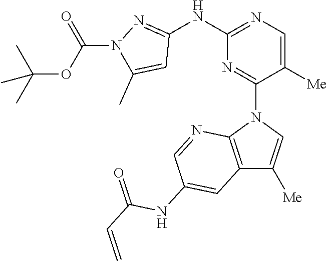 [SMILES: C=CC(=O)Nc1cnc2c(c1)c(C)cn2-c1nc(Nc2cc(C)n(C(=O)OC(C)(C)C)n2)ncc1C] | LCMS m/z = 489 (M + 1); 1H NMR (300 MHz, DMSO-d6) δ: 10.35 (s, 1H), 10.25 (s, 1H), 8.50 (s, 1H), 8.46 (d, J = 2.3 Hz, 1H), 8.42 (d, J = 2.1 Hz, 1H), 7.62 (s, 1H), 6.91 (s, 1H), 6.47 (dd, J = 17.4, 10.3 Hz, 1H), 6.28 (dd, J = 17.9, 3.0 Hz, 1H), 5.78 (dd, J = 9.9, 2.4 Hz, 1H), 2.43 (s, 3H), 2.31 (s, 3H), 2.11 (s, 3H), 1.55 (s, 9H) |
| Example 125 N-[1-[2-[[1-(2-hydroxyethyl)pyrazol- 4-yl]amino]-5-methyl-pyrimidin-4- yl]-3-methyl-pyrrolo[2,3-b]pyridin-5- yl]prop-2-enamide | 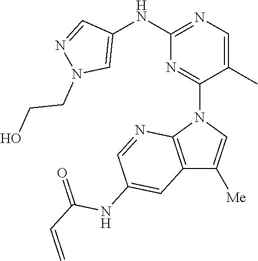 [SMILES: C=CC(=O)Nc1cnc2c(c1)c(C)cn2-c1nc(Nc2cnn(CCO)c2)ncc1C] | LCMS m/z = 419 (M + 1); 1H NMR (300 MHz, DMSO-d6) δ: 10.36 (s, 1H), 9.50 (s, 1H), 8.45 (s, 2H), 8.41 (s, 1H), 7.89 (s, 1H), 7.63 (s, 1H), 7.52 (s, 1H), 6.47 (dd, J = 17.1, 9.9 Hz, 1H), 6.25 (dd, J = 17.1, 2.1 Hz, 1H), 5.74 (dd, J = 9.9, 2.1 Hz, 1H), 4.06 (t, J = 5.4 Hz, 2H), 3.68 (t, J = 5.1 Hz, 2H), 2.32 (s, 3H), 2.10 (s, 3H) |
| Example 126 N-[3-[2-[[1-(2-hydroxyethyl)pyrazol- 4-yl]amino]-5-methyl-pyrimidin-4- yl]-1-methyl-indol-6-yl]prop-2- enamide | 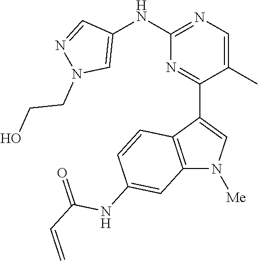 [SMILES: C=CC(=O)Nc1ccc2c(-c3nc(Nc4cnn(CCO)c4)ncc3C)cn(C)c2c1] | LCMS m/z = 418 (M + 1); 1H NMR (600 MHz, DMdSO-d6) δ: 9.91 (br.s, 1H), 8.68 (br.s, 1H), 8.41 (d, J = 8.5 Hz, 1H), 8.18 (s, 1H), 8.04 (s, 1H), 7.87 (s, 1H), 7.87 (d, J = 11.7 Hz, 1H), 7.53 (s, 1H), 7.25 (dd, J = 8.5, 1.8 Hz, 1H), 6.51- 6.44 (m, 1H), 6.26 (dd, J = 17.0, 1.9 Hz, 1H), 5.71 (dd, J = 10.2, 1.9 Hz, 1H), 4.54 (bs, 1H), 4.07 (t, J = 5.7 Hz, 2H), 3.84 (s, 3H), 3.74 (t, J = 5.8 Hz, 2H), 2.33 (s, 3H) |
| Example 127 N-[3-[5-chloro-2-[[1-(2- hydroxyethyl)pyrazol-4- yl]amino]pyrimidin-4-yl]-1-methyl- indol-6-yl]prop-2-enamide | 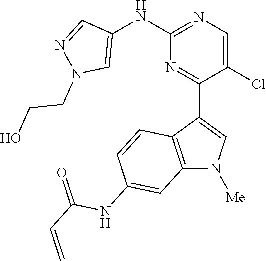 [SMILES: C=CC(=O)Nc1ccc2c(-c3nc(Nc4cnn(CCO)c4)ncc3Cl)cn(C)c2c1] | LCMS m/z = 438, 440 (1Cl isotopic distribution) (M + 1); 1H NMR (400 MHz, DMSO-d6) δ: 10.05 - 9.83 (m, 1H), 9.10 (br d, J = 2.0 Hz, 1H), 8.51 - 8.42 (m, 1H), 8.38 (s, 1H), 8.35 (s, 1H), 8.11 - 8.03 (m, 1H), 7.88 (s, 1H), 7.56 (s, 1H), 7.31 (dd, J = 8.7, 1.8 Hz, 1H), 6.60 - 6.41 (m, 1H), 6.33 - 6.23 (m, 1H), 5.73 (dd, J = 10.1, 1.8 Hz, 1H), 4.57 (br s, 1H), 4.11 (t, J = 5.8 Hz, 2H), 3.86 (s, 3H), 3.81 - 3.73 (m, 2H) |
| Example 128 N-[3-[5-fluoro-2-[[1-(2- hydroxyethyl)pyrazol-4- yl]amino]pyrimidin-4-yl]-1-methyl- indol-6-yl]prop-2-enamide | 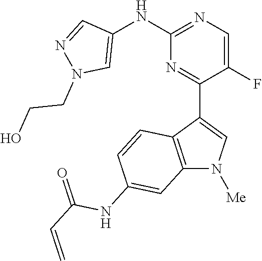 [SMILES: C=CC(=O)Nc1ccc2c(-c3nc(Nc4cnn(CCO)c4)ncc3F)cn(C)c2c1] | LCMS m/z = 422 (M + 1); 1H NMR (300 MHz, DMSO-d6) δ: 10.27 (s, 1H), 9.30 (s, 1H), 8.64 (br s, 1H), 8.35 (d, J = 3.9 Hz, 1H), 8.18 (s, 1H), 8.15 (d, J = 2.7 Hz, 1H), 7.93 (s, 1H), 7.52 (s, 1H), 7.30 (d, J = 9.3 Hz, 1H), 6.50 (dd, J = 17.1, 9.9 Hz, 1H), 6.27 (dd, J = 17.1, 2.1 Hz, 1H), 5.76 (dd, J = 9.9, 2.1 Hz, 1H), 4.10 (t, J = 5.7 Hz, 2H), 3.86 (s, 3H), 3.72 (t, J = 5.7 Hz, 2H) |
| Example 129 N-[1-[5-fluoro-2-[[1-(2- hydroxyethyl)pyrazol-4- yl]amino]pyrimidin-4-yl]-3-methyl- indol-5-yl]prop-2-ynamide | 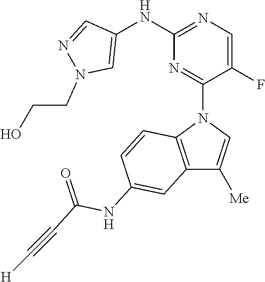 [SMILES: C#CC(=O)Nc1ccc2c(c1)c(C)cn2-c1nc(Nc2cnn(CCO)c2)ncc1F] | LCMS m/z = 420 (M + 1); 1H NMR (DMSO-d6, 600 MHz) δ: 10.86 (s, 1H), 9.59 (s, 1H), 8.58 (br s, 1H), 8.56 (d, J = 4.36 Hz, 1H), 7.99 (d, J = 1.85 Hz, 1H), 7.91 (s, 1H), 7.68 (s, 1H), 7.53 (s, 1H), 7.42 (s, 1H), 4.89-4.86 (m, 1H), 4.41 (s, 1H), 4.12-4.08 (m, 2H), 3.74- 3.70 (m, 2H), 2.27 (s, 3H). |
| Example 130 N-[3-methyl-1-[5-methyl-2-[(2- methylpyrazol-3-yl)amino]pyrimidin- 4-yl]indol-5-yl]but-2-ynamide | 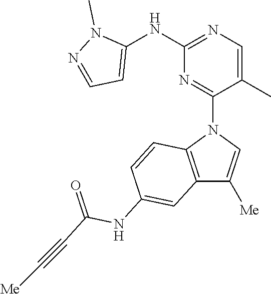 [SMILES: CC#CC(=O)Nc1ccc2c(c1)c(C)cn2-c1nc(Nc2ccnn2C)ncc1C] | LCMS m/z = 400 (M + 1); 1H NMR (400 MHz, DMSO-d6) δ: 10.54 (s, 1H), 9.36 (s, 1H), 8.43 (s, 1H), 7.92 (d, J = 1.8 Hz, 1H), 7.63 (d, J = 8.9 Hz, 1H), 7.55 (d, J = 1.1 Hz, 1H), 7.32 (d, J = 2.0 Hz, 1H), 7.26 (dd, J = 9.0, 1.9 Hz, 1H), 6.21 (d, J = 2.0 Hz, 1H), 3.65 (s, 3H), 2.25 (d, J = 1.0 Hz, 3H), 2.21 (s, 3H), 2.04 (s, 3H) |
| Example 131 N-[3-methyl-1-[5-methyl-2-[(2- methylpyrazol-3-yl)amino]pyrimidin- 4-yl]pyrrolo[2,3-b]pyridin-5-yl]but-2- ynamide | 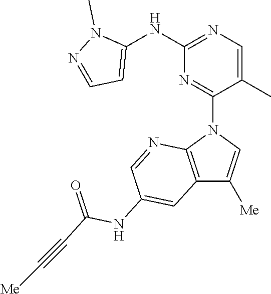 [SMILES: CC#CC(=O)Nc1cnc2c(c1)c(C)cn2-c1nc(Nc2ccnn2C)ncc1C] | LCMS m/z = 401 (M + 1); 1H NMR (DMSO-d6, 300 MHz) δ: 10.76 (s, 1H), 9.49 (s, 1H), 8.49 (s, 1H), 8.30-8.35 (m, 2H), 7.56 (d, 1H, J = 1.0 Hz), 7.29 (d, 1H, J = 1.9 Hz), 6.23 (d, 1H, J = 1.7 Hz), 3.67 (s, 3H), 2.28 (d, 3H, J = 0.7 Hz), 2.08 (s, 3H), 2.05 (s, 3H) |
| Example 132 N-[3-methyl-1-[5-methyl-2-[(2- methylpyrazol-3-yl)amino]pyrimidin- 4-yl]pyrrolo[2,3-b]pyridin-5-yl]prop- 2-ynamide | 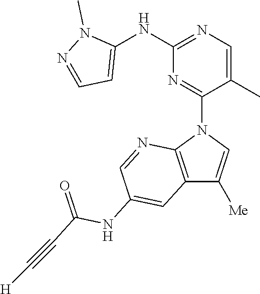 [SMILES: C#CC(=O)Nc1cnc2c(c1)c(C)cn2-c1nc(Nc2ccnn2C)ncc1C] | LCMS m/z = 387 (M + 1); 1H NMR (DMSO-d6, 500 MHz) δ: 10.98 (br s, 1H), 9.50 (s, 1H), 8.51 (s, 1H), 8.37 (d, 1H, J = 2.1 Hz), 8.32 (d, 1H, J = 2.1 Hz), 7.59 (s, 1H), 7.30 (d, 1H, J = 1.8 Hz), 6.24 (d, 1H, J = 1.5 Hz), 4.44 (s, 1H), 3.68 (s, 3H), 2.29 (s, 3H), 2.09 (s, 3H) |
| Example 133 N-[1-[5-fluoro-2-[[1-(2- hydroxyethyl)pyrazol-4- yl]amino]pyrimidin-4-yl]-3-methyl- pyrrolo[2,3-b]pyridin-5-yl]prop-2- ynamide | 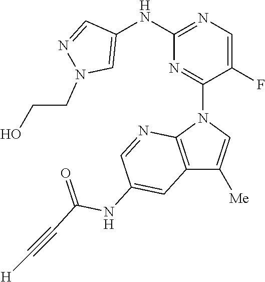 [SMILES: C#CC(=O)Nc1cnc2c(c1)c(C)cn2-c1nc(Nc2cnn(CCO)c2)ncc1F] | LCMS m/z = 421 (M + 1); 1H NMR (500 MHz, DMSO-d6) δ: 11.05 (br.s, 1H), 9.71 (br.s, 1H), 8.64 (d, J = 2.9 Hz, 1H), 8.44 (s, 1H), 8.36 (d, J = 2.2 Hz, 1H), 8.07 (bs, 1H), 7.72 (s, 1H), 7.55 (s, 1H), 4.85 (t, J = 5.1 Hz, 1H), 4.47 (s, 1H), 4.08 (t, J = 5.7 Hz, 2H), 3.70 (q, J = 5.5 Hz, 2H), 2.31 (s, 3H) |
| Example 134 N-[1-[5-fluoro-2-[(2-methylpyrazol-3- yl)amino]pyrimidin-4-yl]-3-methyl- pyrrolo[2,3-b]pyridin-5-yl]but-2- ynamide | 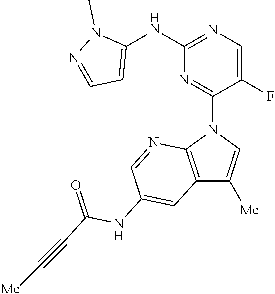 [SMILES: CC#CC(=O)Nc1cnc2c(c1)c(C)cn2-c1nc(Nc2ccnn2C)ncc1F] | LCMS m/z = 405 (M + 1); 1H NMR (500 MHz, DMSO-d6) δ: 10.81 (br.s, 1H), 9.68 (br.s, 1H), 8.70 (d, J = 2.5 Hz, 1H), 8.38 (d, J = 2.5 Hz, 1H), 8.34 (d, J = 2.0 Hz, 1H), 7.62 (s, 1H), 7.33 (d, J = 1.9 Hz, 1H), 6.31 (d, J = 1.7 Hz, 1H), 3.70 (s, 3H), 2.29 (s, 3H), 2.06 (s, 3H) |
| Example 135 N-[3-[5-chloro-2-[[1-(2- hydroxyethyl)pyrazol-4- yl]amino]pyrimidin-4-yl]-1-methyl- indol-6-yl]but-2-ynamide | 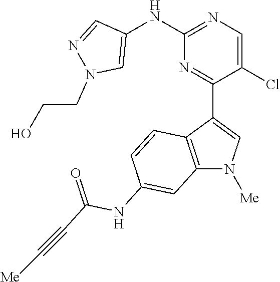 [SMILES: CC#CC(=O)Nc1ccc2c(-c3nc(Nc4cnn(CCO)c4)ncc3Cl)cn(C)c2c1] | LCMS m/z = 450 (M + 1); 1H NMR (500 MHz, DMSO-d6) δ: 10.31 (br.s, 1H), 9.09 (br.s, 1H), 8.46 (d, J = 8.4 Hz, 1H), 8.38 (s, 1H), 8.35 (s, 1H), 7.93 (br.s, 1H), 7.88 (s, 1H), 7.56 (s, 1H), 7.28 (d, J = 8.9 Hz, 1H), 4.56 (t, J = 5.1 Hz, 1H), 4.11 (t, J = 5.4 Hz, 2H), 3.85 (s, 3H), 3.77 (q, J = 5.4 Hz, 2H), 2.05 (s, 3H) |
| Example 136 N-[3-ethyl-1-[5-methyl-2-[(1- methylpyrazol-4-yl)amino]pyrimidin- 4-yl]indol-5-yl]prop-2-enamide | 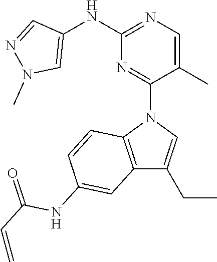 [SMILES: C=CC(=O)Nc1ccc2c(c1)c(CC)cn2-c1nc(Nc2cnn(C)c2)ncc1C] | LCMS m/z = 402 (M + 1); 1H NMR (DMSO-d6, 600 MHz) δ: 10.14 (s, 1H), 9.47 (br s, 1H), 8.43 (s, 1H), 8.09 (s, 1H), 7.70-7.90 (m, 2H), 7.53 (s, 1H), 7.48 (s, 1H), 7.40-7.42 (m, 1H), 6.47 (dd, 1H, J = 10.2, 17.1 Hz), 6.26 (d, 1H, J = 16.9 Hz), 5.74 (d, 1H, J = 10.3 Hz), 3.76 (br s, 3H), 2.74 (d, 2H, J = 7.5 Hz), 2.21 (s, 3H), 1.32 (t, 3H, J = 7.5 Hz) |
| Example 137 N-[1-[2-[[1-(2-hydroxyethyl)pyrazol- 4-yl]amino]pyrimidin-4-yl]-3-methyl- indol-5-yl]prop-2-enamide | 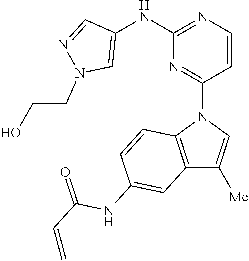 [SMILES: C=CC(=O)Nc1ccc2c(c1)c(C)cn2-c1ccnc(Nc2cnn(CCO)c2)n1] | LCMS m/z = 404 (M + 1); 1H NMR (DMSO-d6, 600 MHz) δ: 10.19 (s, 1H), 9.53 (br s, 1H), 8.87 (br s, 1H), 8.38 (d, J = 5.93 Hz, 1H), 8.06 (s, 1H), 7.97 (br s, 1H), 7.91 (s, 1H), 7.55 (s, 1H), 7.48 (br s, 1H), 7.01 (d, J = 5.60 Hz, 1H), 6.48 (dd, J = 10.21, 16.96 Hz, 1H), 6.27 (d, J = 17.13 Hz, 1H), 5.76 (d, J = 10.37 Hz, 1H), 4.86 (br s, 1H), 4.14- 4.10 (m, 2H), 3.76-3.72 (m, 2H), 2.28 (s, 3H). |
| Example 138 N-[1-[5-fluoro-2-[(2-methylpyrazol-3- yl)amino]pyrimidin-4-yl]-3-methyl- indol-5-yl]prop-2-ynamide | 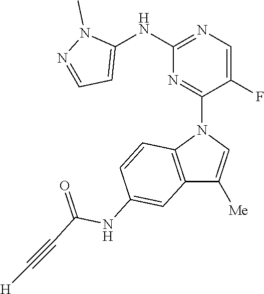 [SMILES: C#CC(=O)Nc1ccc2c(c1)c(C)cn2-c1nc(Nc2ccnn2C)ncc1F] | LCMS m/z = 390 (M + 1); 1H NMR (DMSO-d6, 500 MHz) δ: 10.83 (s, 1H), 9.46 (s, 1H), 8.55 (d, J = 4.93 Hz, 1H), 8.08 (br s, 1H), 7.94 (d, J = 2.00 Hz, 1H), 7.71-7.69 (m, 1H), 7.43 (d, J = 1.84 Hz, 1H), 7.30 (d, J = 9.00 Hz, 1H), 6.22 (d, J = 1.82 Hz, 1H), 4.39 (s, 1H), 3.65 (s, 3H), 2.25 (s, 3H). |
| Example 139 N-[1-[5-fluoro-2-[(2-methylpyrazol-3- yl)amino]pyrimidin-4-yl]-3-methyl- indol-5-yl]but-2-ynamide | 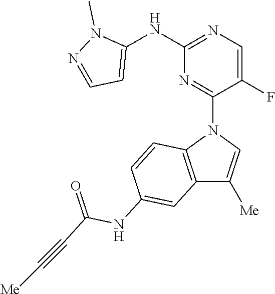 [SMILES: CC#CC(=O)Nc1ccc2c(c1)c(C)cn2-c1nc(Nc2ccnn2C)ncc1F] | LCMS m/z = 404 (M + 1); 1H NMR (DMSO-d6, 500 MHz) δ: 10.61 (s, 1H), 9.45 (s, 1H), 8.55 (d, J = 4.79 Hz, 1H), 8.07 (br s, 1H), 7.94 (d, J = 1.83 Hz, 1H), 7.70-7.68 (m, 1H), 7.42 (d, J = 1.84 Hz, 1H), 7.28 (d, J = 9.06 Hz, 1H), 6.22 (d, J = 1.82 Hz, 1H), 3.65 (s, 3H), 2.25 (s, 3H), 2.05 (s, 3H). |
| Example 140 N-[1-[2-[[1-(2-hydroxyethyl)pyrazol- 4-yl]amino]-5-methyl-pyrimidin-4- yl]-3-methyl-indol-5-yl]but-2- ynamide | 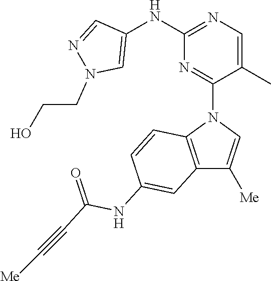 [SMILES: CC#CC(=O)Nc1ccc2c(c1)c(C)cn2-c1nc(Nc2cnn(CCO)c2)ncc1C] | LCMS m/z = 430 (M + 1); 1H NMR (DMSO-d6, 600 MHz) δ: 10.57 (s, 1H), 9.46 (br s, 1H), 8.42 (s, 1H), 7.97 (s, 1H), 7.83 (br s, 1H), 7.74 (br s, 1H), 7.56 (s, 1H), 7.51 (s, 1H), 7.32 (d, J = 9.2 Hz, 1H), 4.04 (br s, 2H), 3.68 (br s, 2H), 3.54 (br s, OH), 2.28 (s, 3H), 2.20 (s, 3H), 2.05 (s, 3H) |
| Example 141 N-[1-[2-[[1-(2-hydroxyethyl)pyrazol- 4-yl]amino]-5-methyl-pyrimidin-4- yl]-3-methyl-pyrrolo[2,3-b]pyridin-5- yl]prop-2-ynamide | 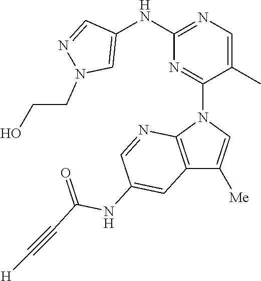 [SMILES: C#CC(=O)Nc1cnc2c(c1)c(C)cn2-c1nc(Nc2cnn(CCO)c2)ncc1C] | LCMS m/z = 417 (M + 1); ¹H NMR ¹HNMR (600 MHz, DMSO-d6) δ: 11.00 (s, 1H), 9.50 (s, 1H), 8.46 (s, 1H), 8.39 (s, 1H), 8.34 (s, 1H), 7.90 (s, 1H), 7.66 (br.s, 1H), 7.52 (s, 1H), 4.85 (t, J = 4.8 Hz, 1H), 4.45 (s, 1H), 4.07 (br.s, 2H), 3.70 (dd, J = 10.2, 4.8 Hz, 2H), 2.32 (s, 3H), 2.10 (s, 3H) |
| Example 142 N-[3-[5-fluoro-2-[[1-(2- hydroxyethyl)pyrazol-4- yl]amino]pyrimidin-4-yl]-1-methyl- indol-6-yl]prop-2-ynamide | 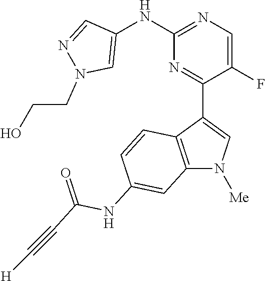 [SMILES: C#CC(=O)Nc1ccc2c(-c3nc(Nc4cnn(CCO)c4)ncc3F)cn(C)c2c1] | LCMS m/z = 420 (M + 1); ¹H NMR (600 MHz, DMSO-d6) δ: 10.91 (s, 1H), 9.29 (s, 1H), 8.68 (br.s, 1H), 8.36 (d, J = 3.6 Hz, 1H), 8.17 (d, J = 3.0 Hz, 1H), 8.03 (d, J = 1.8 Hz, 1H), 7.93 (s, 1H), 7.52 (s, 1H), 7.30 (d, J = 8.4 Hz, 1H), 4.88 (t, J = 5.4 Hz, 1H), 4.42 (s, 1H), 4.10 (t, J = 5.4 Hz, 2H), 3.86 (s, 3H), 3.73 (dd, J = 11.4, 5.4 Hz, 2H) |
| Example 143 N-[3-[2-[[1-(2-hydroxyethyl)pyrazol- 4-yl]amino]pyrimidin-4-yl]-1-methyl- indol-6-yl]prop-2-ynamide | 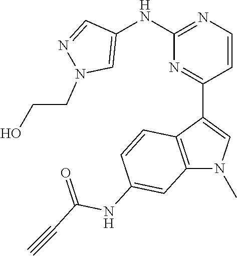 [SMILES: C#CC(=O)Nc1ccc2c(-c3ccnc(Nc4cnn(CCO)c4)n3)cn(C)c2c1] | LCMS m/z = 402 (M + 1); 1H NMR (600 MHz, DMSO-d6) δ 10.55 (br s, 1H), 8.85 (br s, 1H), 8.40 (br d, J = 8.6 Hz, 1H), 8.26 (d, J = 5.3 Hz, 1H), 8.12 (s, 1H), 7.94 (s, 1H), 7.90 (br s, 1H), 7.56 (s, 1H), 7.29 (br d, J = 8.4 Hz, 1H), 7.02 (d, J = 5.3 Hz, 1H), 4.60 (br s, 1H), 4.17 - 4.13 (m, 1H), 4.13 - 4.10 (m, 2H), 3.81 (s, 3H), 3.77 (q, J = 5.5 Hz, 2H) |
| Example 144 (E)-4-(dimethylamino)-N-[1-[5- fluoro-2-[[1-(2-hydroxyethyl)pyrazol- 4-yl]amino]pyrimidin-4-yl]-3-methyl- indol-5-yl]but-2-enamide | 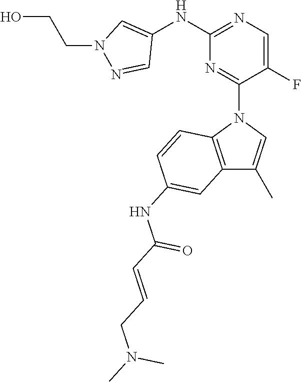 [SMILES: Cc1cn(-c2nc(Nc3cnn(CCO)c3)ncc2F)c2ccc(NC(=O)/C=C/CN(C)C)cc12] | LCMS m/z = 479 (M + 1); 1H NMR (DMSO-d6, 500 MHz) δ: 10.19 (s, 1H), 9.58 (s, 1H), 8.55 (d, J = 4.60 Hz, 1H), 8.51 (br s, 1H), 8.09 (s, 1H), 7.91 (s, 1H), 7.67 (s, 1H), 7.53 (s, 1H), 7.45 (br s, 1H), 6.75 (s, 1H), 6.33 (s, 1H), 4.12- 4.07 (m, 2H), 3.74-3.70 (m, 2H), 3.15 (br s, 2H), 2.28 (s, 3H), 2.25 (s, 6H). |
| Example 145 N-[3-[2-[[1-(2-hydroxyethyl)pyrazol- 4-yl]amino]pyrimidin-4-yl]-1-methyl- indol-6-yl]but-2-ynamide | 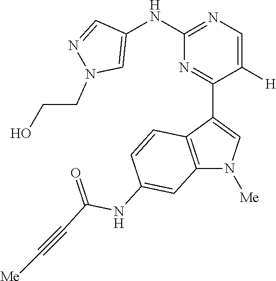 [SMILES: CC#CC(=O)Nc1ccc2c(-c3ccnc(Nc4cnn(CCO)c4)n3)cn(C)c2c1] | LCMS m/z = 416 (M + 1); 1H NMR (600 MHz, DMSO-d6) δ: 10.30 (br s, 1H), 8.84 (br s, 1H), 8.38 (br d, J = 8.8 Hz, 1H), 8.26 (d, J = 5.1 Hz, 1H), 8.10 (s, 1H), 7.93 (s, 1H), 7.90 (br s, 1H), 7.56 (d, J = 0.7 Hz, 1H), 7.27 (d, J = 8.6 Hz, 1H), 7.02 (d, J = 5.3 Hz, 1H), 4.60 (br s, 1H), 4.12 (t, J = 5.8 Hz, 2H), 3.80 (s, 3H), 3.77 (q, J = 5.7 Hz, 2H), 2.03 (s, 3H) |
| Example 146 N-[1-[2-[(1,5-dimethylpyrazol-4- yl)amino]-5-fluoro-pyrimidin-4-yl]-3- methyl-indol-5-yl]prop-2-enamide | 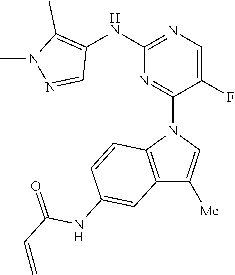 [SMILES: C=CC(=O)Nc1ccc2c(c1)c(C)cn2-c1nc(Nc2cnn(C)c2C)ncc1F] | LCMS m/z = 406 (M + 1); 1H NMR (500 MHz, DMSO-d6) δ: 9.88 (br s, 1H), 8.45 (s, 1H), 8.39 (d, J = 4.9 Hz, 1H), 8.10 (br d, J = 8.5 Hz, 1H), 7.98 (s, 1H), 7.63 (s, 1H), 7.42 (s, 1H), 7.38 (br d, J = 8.9 Hz, 1H), 6.47 (dd, J = 16.9, 10.2 Hz, 1H), 6.27 (br d, J = 17.1 Hz, 1H), 5.72 (br d, J = 10.1 Hz, 1H), 3.77 (s, 3H), 2.28 (s, 3H), 2.15 (s, 3H). |
| Example 147 N-[1-[2-[(1,5-dimethylpyrazol-4- yl)amino]-5-methyl-pyrimidin-4-yl]- 3-methyl-indol-5-yl]prop-2-enamide | 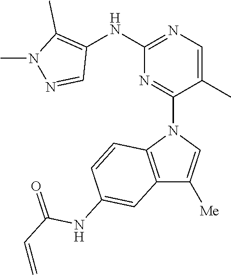 | LCMS m/z = 402 (M + 1); 1H NMR (DMSO-d6) δ: 10.12 (s, 1H), 8.65 (s, 1H), 8.33 (s, 1H), 8.02 (s, 1H), 7.68 (br s, 1H), 7.53 (s, 1H), 7.47 (s, 1H), 7.30- 7.35 (bd, 1H), 6.46 (dd, J = 17.1, 10.1 Hz, 1H), 6.26 (dd, J = 17.1, 1.8 Hz, 1H), 5.74 (dd, J = 10.1, 1.8 Hz, 1H), 3.69 (s, 3H), 2.27 (s, 3H), 2.18 (s, 3H), 2.14 (s, 3H) |
| Example 148 N-[1-[5-fluoro-2-(isothiazol-4- ylamino)pyrimidin-4-yl]-3-methyl- indol-5-yl]prop-2-enamide | 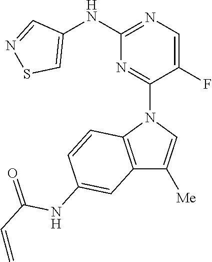 [SMILES: C=CC(=O)Nc1ccc2c(c1)c(C)cn2-c1nc(Nc2cnsc2)ncc1F] | LCMS m/z = 395 (M + 1); 1H NMR (500 MHz, DMSO-d6): 10.35 (br.s, 1H), 10.23 (br.s, 1H), 8.86 (s, 1H), 8.68 (s, 1H), 8.65 (d, J = 4.5 Hz, 1H), 8.41 (br.s, 1H), 8.09 (d, J = 1.5 Hz, 1H), 7.70 (s, 1H), 7.49 (d, J = 8.8 Hz, 1H), 6.53- 6.42 (m, 1H), 6.27 (dd, J = 17.0, 1.8 Hz, 1H), 5.76 (dd, J = 10.1, 1.6 Hz, 1H), 2.29 (s, 3H) |
| Example 149 N-[1-[2-[[1-(difluoromethyl)-3- methyl-pyrazol-4-yl]amino]-5-fluoro- pyrimidin-4-yl]-3-methyl-indol-5- yl]prop-2-enamide | 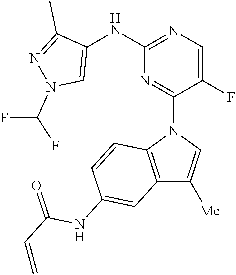 [SMILES: C=CC(=O)Nc1ccc2c(c1)c(C)cn2-c1nc(Nc2cn(C(F)F)nc2C)ncc1F] | LCMS m/z = 442 (M + 1); 1H NMR (DMSO-d6, 600 MHz) δ: 9.89 (s, 1H), 8.84 (s, 1H), 8.49 (d, J = 4.8 Hz, 1H), 8.23 (s, 1H), 8.19 (br d, J = 8.8 Hz, 1H), 7.99 (d, J = 1.8 Hz, 1H), 7.61-7.65 (m, 1H), 7.56 (t, J = 60.2 Hz, 1H), 7.41 (dd, J = 9.0, 2.0 Hz, 1H), 6.46 (dd, J = 17.0, 10.2 Hz, 1H), 6.26 (dd, J = 17.0, 1.9 Hz, 1H), 5.71 (dd, J = 10.3, 2.0 Hz, 1H), 2.28 (d, J = 1.1 Hz, 3H), 2.23 (s, 3H) |
| Example 150 N-[1-[2-[(2,4-dimethylpyrimidin-5- yl)amino]-5-fluoro-pyrimidin-4-yl]-3- methyl-indol-5-yl]prop-2-enamide | 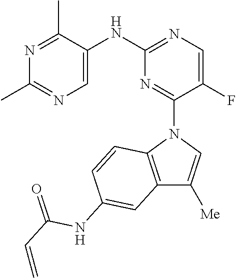 [SMILES: C=CC(=O)Nc1ccc2c(c1)c(C)cn2-c1nc(Nc2cnc(C)nc2C)ncc1F] | LCMS m/z = 418 (M + 1); 1H NMR (300 MHz, DMSO-d6) δ: 10.18 (s, 1H), 9.29 (s, 1H), 8.65 (s, 1H), 8.51 (d, J = 4.9 Hz, 1H), 8.04 (d, J = 1.4 Hz, 2H), 7.67 (s, 1H), 7.32 (br d, J = 1.7 Hz, 1H), 6.46 (dd, J = 16.8, 10.2 Hz, 1H), 6.26 (dd, J = 16.8, 2.2 Hz, 1H), 5.78 - 5.71 (m, 1H), 2.61 (s, 3H), 2.41 - 2.37 (m, 3H), 2.25 (s, 3H) |
| Example 151 N-methyl-N-[3-methyl-1-[5-methyl-2- [(2-methylpyrazol-3- yl)amino]pyrimidin-4-yl]indol-5- yl]prop-2-enamide | 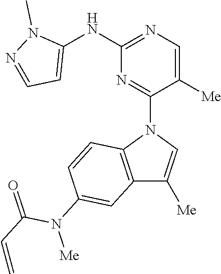 [SMILES: C=CC(=O)N(C)c1ccc2c(c1)c(C)cn2-c1nc(Nc2ccnn2C)ncc1C] | LCMS m/z = 402 (M + 1); ¹H NMR (600 MHz, DMSO-d6) δ: 9.41 (s, 1H), 8.48 (s, 1H), 7.76 (d, J = 8.4 Hz, 1H), 7.65 (s, 1H), 7.50 (s, 1H), 7.33 (s, 1H), 7.06 (dd, J = 8.4, 2.4 Hz, 1H), 6.22 (s, 1H), 6.15 (dd, J = 16.8, 2.4 Hz, 1H), 6.01-6.08 (m, 1H), 5.32 (d, J = 10.2 Hz, 1H), 3.67 (s, 3H), 3.30 (s, 3H), 2.29 (s, 3H), 2.22 (s, 3H) |
| Example 152 N-methyl-N-[3-methyl-1-[5-methyl-2- [(2-methylpyrazol-3- yl)amino]pyrimidin-4-yl]indol-5- yl]but-2-ynamide | 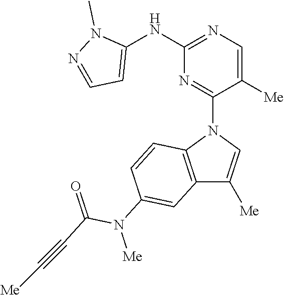 [SMILES: CC#CC(=O)N(C)c1ccc2c(c1)c(C)cn2-c1nc(Nc2ccnn2C)ncc1C] | LCMS m/z = 414 (M + 1); ¹H NMR (600 MHz, DMSO-d6) δ: 9.40 (s, 1H), 8.48 (s, 1H), 7.71 (d, J = 9.0 Hz, 1H), 7.63 (s, 1H), 7.57 (s, 1H), 7.33 (s, 1H), 7.13 (dd, J = 9.0, 2.4 Hz, 1H), 6.23 (s, 1H), 3.67 (s, 3H), 3.25 (s, 3H), 2.31 (s, 3H), 2.23 (s, 3H), 1.72 (s, 3H) |
| Example 153 N-[1-[5-fluoro-2-[(2-methyl-3- pyridyl)amino]pyrimidin-4-yl]-3- methyl-indol-5-yl]prop-2-enamide | 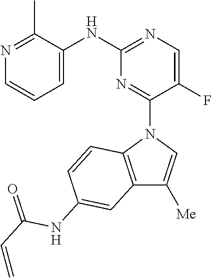 [SMILES: C=CC(=O)Nc1ccc2c(c1)c(C)cn2-c1nc(Nc2cccnc2C)ncc1F] | LCMS m/z = 403 (M + 1); 1H NMR (DMSO-d6, 500 MHz) δ: 10.17 (s, 1H), 9.22 (s, 1H), 8.52 (d, J = 5.00 Hz, 1H), 8.35- 8.31 (m, 1H), 8.06-7.99 (m, 2H), 7.83 (d, J = 7.95 Hz, 1H), 7.68 (s, 1H), 7.31-7.22 (m, 2H), 6.46 (dd, J = 10.10, 16.89 Hz, 1H), 6.26 (dd, J = 1.59, 16.91 Hz, 1H), 5.75 (dd, J = 1.58, 10.03 Hz, 1H), 2.44 (s, 3H), 2.26 (s, 3H). |
| Example 154 N-[3-[2-[(1,3-dimethylpyrazol-4- yl)amino]-5-fluoro-pyrimidin-4-yl]-1- methyl-indol-6-yl]prop-2-enamide | 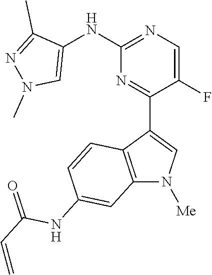 [SMILES: C=CC(=O)Nc1ccc2c(-c3nc(Nc4cn(C)nc4C)ncc3F)cn(C)c2c1] | LCMS m/z = 406 (M + 1); 1H NMR (500 MHz, DMSO-d6): 9.96 (br.s, 1H), 8.38 (d, J = 8.7 Hz, 1H), 8.24 (d, J = 3.7 Hz, 1H), 8.18 (br.s, 1H), 8.09-8.03 (m, 2H), 7.71 (s, 1H), 7.25 (dd, J = 8.5, 1.7 Hz, 1H), 6.52-6.44 (m, 1H), 6.28 (dd, J = 16.8, 1.7 Hz, 1H), 5.73 (dd, J = 10.0, 1.9 Hz, 1H), 3.85 (s, 3H), 3.78 (s, 3H), 2.11 (s, 3H) |
| Example 155 N-[1-[5-fluoro-2-[(3-methylisoxazol- 4-yl)amino]pyrimidin-4-yl]-3-methyl- indol-5-yl]prop-2-enamide | 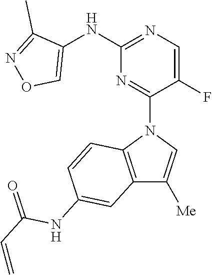 [SMILES: C=CC(=O)Nc1ccc2c(c1)c(C)cn2-c1nc(Nc2conc2C)ncc1F] | LCMS m/z = 393 (M + 1); ¹H NMR (DMSO-d6, 500 MHz) δ: 10.22 (s, 1H), 9.29 (s, 1H), 9.02 (s, 1H), 8.60 (d, J = 4.3 Hz, 1H), 8.32 (br s, 1H), 8.07 (s, 1H), 7.69 (s, 1H), 7.46 (br d, J = 6.7 Hz, 1H), 6.47 (dd, J = 16.9, 10.2 Hz, 1H), 6.28 (br d, J = 16.8 Hz, 1H), 5.76 (br d, J = 10.1 Hz, 1H), 2.30 (s, 3H), 2.28 (s, 3H) |
| Example 156 N-[1-[2-[(1-ethyl-3-methyl-pyrazol-4- yl)amino]-5-fluoro-pyrimidin-4-yl]-3- methyl-indol-5-yl]prop-2-enamide | 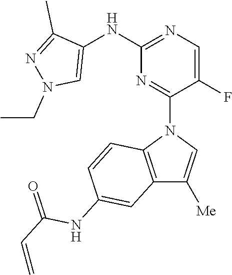 [SMILES: C=CC(=O)Nc1ccc2c(c1)c(C)cn2-c1nc(Nc2cn(CC)nc2C)ncc1F] | LCMS m/z = 420 (M + 1); ¹H NMR (DMSO-d6, 600 MHz) δ: 9.89 (s, 1H), 8.48 (s, 1H), 8.43 (d, J = 5.0 Hz, 1H), 8.16 (d, J = 1.0 Hz, 1H), 7.99 (d, J = 1.8 Hz, 1H), 7.75 (s, 1H), 7.63-7.65 (m, 1H), 7.41 (dd, J = 1.0 Hz, 1H), 6.48 (dd, J = 17.0, 10.2 Hz, 1H), 6.28 (dd, J = 17.0, 1.9 Hz, 1H), 5.73 (dd, J = 10.2, 1.9 Hz, 1H), 4.06 (q, J = 7.3 Hz, 2H), 2.29 (d, J = 1.3 Hz, 3H), 2.12 (s, 3H), 1.41 (t, J = 7.2 Hz, 3H) |
| Example 157 N-[1-[5-fluoro-2-[(2-fluoro-3- pyridyl)amino]pyrimidin-4-yl]-3- methyl-indol-5-yl]prop-2-enamide | 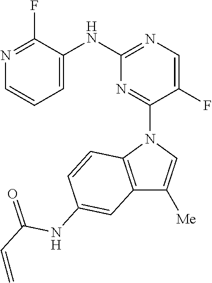 [SMILES: C=CC(=O)Nc1ccc2c(c1)c(C)cn2-c1nc(Nc2cccnc2F)ncc1F] | LCMS m/z = 407 (M + 1); 1H NMR (500 MHz, DMSO-d6) δ: 10.20 (s, 1H), 9.58 (s, 1H), 8.58 (d, J = 4.9 Hz, 1H), 8.32 - 8.24 (m, 2H), 8.06 (d, J = 1.8 Hz, 1H), 8.01 - 7.97 (m, 1H), 7.72 - 7.68 (m, 1H), 7.40 - 7.35 (m, 2H), 6.48 - 6.43 (m, 1H), 6.27 (dd, J = 2.0, 16.9 Hz, 1H), 5.76 (dd, J = 1.9, 10.1 Hz, 1H), 2.29 - 2.25 (m, 3H). |
| Example 158 N-[1-[5-fluoro-2-[[1-(2- hydroxypropyl)pyrazol-4- yl]amino]pyrimidin-4-yl]-3-methyl- indol-5-yl]prop-2-enamide | 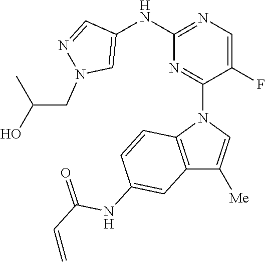 [SMILES: C=CC(=O)Nc1ccc2c(c1)c(C)cn2-c1nc(Nc2cnn(CC(C)O)c2)ncc1F] | LCMS m/z = 436 (M + 1); 1H NMR (DMSO-d6, 600 MHz) δ: 9.91 (s, 1H), 9.24 (s, 1H), 8.48 (d, J = 4.8 Hz, 1H), 8.29 (d, J = 8.4 Hz, 1H), 7.99 (d, J = 1.8 Hz, 1H), 7.85 (s, 1H), 7.62 (dd, J = 2.3, 1.2 Hz, 1H), 7.54 (d, J = 0.7 Hz, 1H), 7.48 (dd, J = 8.9, 1.9 Hz, 1H), 6.47 (dd, J = 17.1, 10.3 Hz, 1H), 6.26 (dd, J = 17.0, 1.9 Hz, 1H), 5.71 (dd, J = 10.2, 1.9 Hz, 1H), 3.92-4.03 (m, 3H), 3.0-3.5 (bs, OH + H2O), 2.29 (d, J = 1.1 Hz, 3H), 1.05 (d, J = 5.7 Hz, 3H) |
| Example 159 N-[1-[2-[[1-(1-cyano-1-methyl-ethyl)- 3-methyl-pyrazol-4-yl]amino]-5- fluoro-pyrimidin-4-yl]-3-methyl- indol-5-yl]prop-2-enamide | 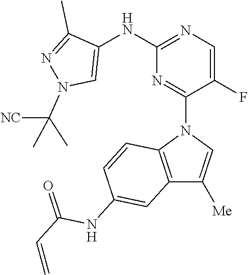 [SMILES: C=CC(=O)Nc1ccc2c(c1)c(C)cn2-c1nc(Nc2cn(C(C)(C)C#N)nc2C)ncc1F] | LCMS m/z = 459 (M + 1); 1H NMR (500 MHz, DMSO-d6) δ: 9.91 (s, 1H), 8.72 (s, 1H), 8.48 (d, J = 4.9 Hz, 1H), 8.16 (d, J = 8.8 Hz, 1H), 8.05 (s, 1H), 7.99 (d, J = 1.8 Hz, 1H), 7.65 (s, 1H), 7.44 (dd, J = 8.9, 1.8 Hz, 1H), 6.47 (dd, J = 16.9, 10.2 Hz, 1H), 6.27 (dd, J = 16.9, 2.0 Hz, 1H), 5.72 (dd, J = 10.1, 1.8 Hz, 1H), 2.29 (s, 3H), 2.19 (s, 3H), 1.95 (s, 6H) |
| Example 160 N-[1-[5-fluoro-2-[[1-(3- hydroxypropyl)pyrazol-4- yl]amino]pyrimidin-4-yl]-3-methyl- indol-5-yl]prop-2-enamide | 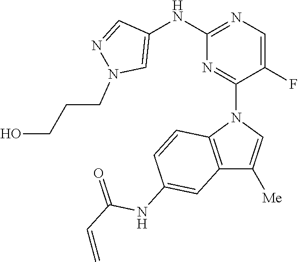 [SMILES: C=CC(=O)Nc1ccc2c(c1)c(C)cn2-c1nc(Nc2cnn(CCCO)c2)ncc1F] | LCMS m/z = 436 (M + 1); 1H NMR (500 MHz, DMSO-d6) δ: 9.94 (s, 1H), 9.26 (s, 1H), 8.49 (d, J = 4.6 Hz, 1H), 8.30 (d, J = 8.2 Hz, 1H), 8.01 (bs, 1H), 7.84 (s, 1H), 7.64 (bs, 1H), 7.54 (s, 1H), 7.49 (d, J = 8.9 Hz, 1H), 6.48 (dd, J = 10.2, 16.9 Hz, 1H), 6.28 (d, J = 17.1 Hz, 1H), 5.73 (d, J = 10.4 Hz, 1H), 4.31 - 4.26 (m, 1H), 4.13 (t, J = 6.9 Hz, 2H), 3.45 (q, J = 5.8 Hz, 2H), 2.30 (s, 3H), 1.95 (quin, J = 6.6 Hz, 2H). |
| Example 161 N-[1-[5-fluoro-2-[(5-methylisothiazol- 3-yl)amino]pyrimidin-4-yl]-3-methyl- indol-5-yl]prop-2-enamide | 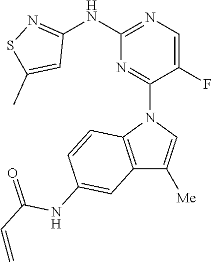 [SMILES: C=CC(=O)Nc1ccc2c(c1)c(C)cn2-c1nc(Nc2cc(C)sn2)ncc1F] | LCMS m/z = 409 (M + 1); 1H NMR (DMSO-d6, 600 MHz) δ: 10.70 (s, 1H), 10.22 (s, 1H), 8.80 (d, J = 9.0 Hz, 1H), 8.62 (d, J = 5.0 Hz, 1H), 8.07 (d, J = 2.0 Hz, 1H), 7.73 (s, 1H), 7.56 (d, J = 0.7 Hz, 1H), 7.48 (dd, J = 9.0, 2.0 Hz, 1H), 6.48 (dd, J = 17.0, 10.2 Hz, 1H), 6.28 (dd, J = 17.0, 1.9 Hz, 1H), 5.69-5.82 (m, 1H), 2.56 (d, J = 0.9 Hz, 3H), 2.29 (d, J = 1.1 Hz, 3H) |
| Example 162 N-[1-[5-fluoro-2-[[1-(2- hydroxyethyl)-3-methyl-pyrazol-4- yl]amino]pyrimidin-4-yl]-3-methyl- indol-5-yl]prop-2-enamide N-[1-[5-fluoro-2-[[1-(2- hydroxyethyl)-5-methyl-pyrazol-4- yl]amino]pyrimidin-4-yl]-3-methyl- indol-5-yl]prop-2-enamide | 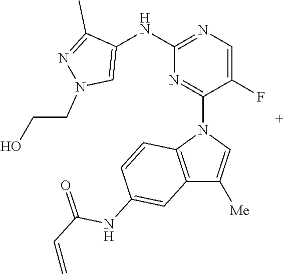 [SMILES: C=CC(=O)Nc1ccc2c(c1)c(C)cn2-c1nc(Nc2cn(CCO)nc2C)ncc1F] | LCMS m/z = 436 (M + 1). |
| | 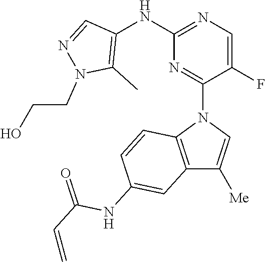 [SMILES: C=CC(=O)Nc1ccc2c(c1)c(C)cn2-c1nc(Nc2cnn(CCO)c2C)ncc1F] | |
| Example 163 N-[1-[5-fluoro-2-[[1-(2- methoxyethyl)pyrazol-4- yl]amino]pyrimidin-4-yl]-3-methyl- indol-5-yl]prop-2-enamide | 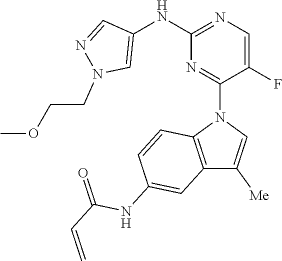 [SMILES: C=CC(=O)Nc1ccc2c(c1)c(C)cn2-c1nc(Nc2cnn(CCOC)c2)ncc1F] | LCMS m/z = 436 (M + 1); 1H NMR (DMSO-d6, 600 MHz) δ: 9.90 (s, 1H), 9.21 (br s, 1H), 8.47 (d, J = 4.6 Hz, 1H), 8.27 (br d, J = 8.8 Hz, 1H), 7.99 (d, J = 1.8 Hz, 1H), 7.84 (d, J = 0.6 Hz, 1H), 7.61 (dd, J = 2.4, 1.3 Hz, 1H), 7.54 (s, 1H), 7.49 (dd, J = 8.9, 2.1 Hz, 1H), 6.44- 6.49 (m, 1H), 6.26 (dd, J = 17.1, 2.0 Hz, 1H), 5.71 (dd, J = 1.0 Hz, 1H), 4.20 (t, J = 5.6 Hz, 2H), 3.69 (t, J = 1.0 Hz, 2H), 3.24 (s, 3H), 2.29 (d, J = 1.1 Hz, 3H) |
| Example 164 N-[1-[2-[[1-(2,2-difluoroethyl)-3- methyl-pyrazol-4-yl]amino]-5-fluoro- pyrimidin-4-yl]-3-methyl-indol-5- yl]prop-2-enamide | 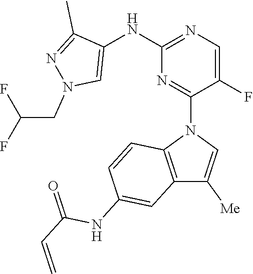 [SMILES: C=CC(=O)Nc1ccc2c(c1)c(C)cn2-c1nc(Nc2cn(CC(F)F)nc2C)ncc1F] | LCMS m/z = 456 (M + 1); 1H NMR (500 MHz, DMSO-d6) δ: 10.19 (s, 1H), 8.92 (s, 1H), 8.50 (d, J = 4.5 Hz, 1H), 8.07 (s, 1H), 7.90 (s, 1H), 7.67 (s, 1H), 7.38 (br.s, 2H), 6.47 (dd, J = 17.0, 10.0 Hz, 1H), 6.28 (dt, J = 51.0, 4.0 Hz, 1H), 6.27 (dd, J = 17.0, 2.0 Hz, 1H), 5.75 (dd, J = 10.0, 2.0 Hz, 1H), 4.49 (dt, J = 15.0, 4.0 Hz, 2H), 2.27 (s, 3H), 2.13 (s, 3H) |
| Example 165 N-[1-[5-fluoro-2-[[3-methyl-1-(2,2,2- trifluoroethyl)pyrazol-4- yl]amino]pyrimidin-4-yl]-3-methyl- indol-5-yl]prop-2-enamide | 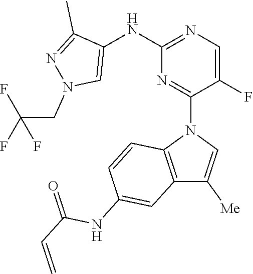 [SMILES: C=CC(=O)Nc1ccc2c(c1)c(C)cn2-c1nc(Nc2cn(CC(F)(F)F)nc2C)ncc1F] | LCMS m/z = 474 (M + 1); 1H NMR (500 MHz, DMSO-d6) δ: 9.91 (s, 1H), 8.67 (s, 1H), 8.46 (d, J = 4.6 Hz, 1H), 8.18 (br d, J = 8.9 Hz, 1H), 8.00 (s, 1H), 7.93 (s, 1H), 7.64 (s, 1H), 7.42 (br d, J = 9.2 Hz, 1H), 6.48 (dd, J = 16.9, 10.2 Hz, 1H), 6.27 (dd, J = 17.1, 1.8 Hz, 1H), 5.72 (dd, J = 10.2, 1.7 Hz, 1H), 4.94 (q, J = 9.2 Hz, 2H), 2.29 (s, 3H), 2.16 (s, 3H) |
| Example 166 N-[1-[2-[[1-[2-(dimethylamino)ethyl]- 3-methyl-pyrazol-4-yl]amino]-5- fluoro-pyrimidin-4-yl]-3-methyl- indol-5-yl]prop-2-enamide N-[1-[2-[[1-[2-(dimethylamino)ethyl]- 5-methyl-pyrazol-4-yl]amino]-5- fluoro-pyrimidin-4-yl]-3-methyl- indol-5-yl]prop-2-enamide | 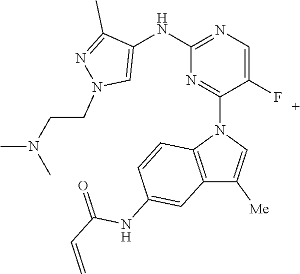 [SMILES: C=CC(=O)Nc1ccc2c(c1)c(C)cn2-c1nc(Nc2cn(CCN(C)C)nc2C)ncc1F] | LCMS m/z = 463 (M + 1). |
| | 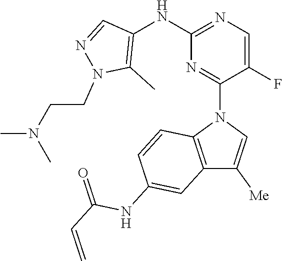 [SMILES: C=CC(=O)Nc1ccc2c(c1)c(C)cn2-c1nc(Nc2cnn(CCN(C)C)c2C)ncc1F] | |
| Example 167 N-[1-[2-[(1,3-dimethylpyrazol-4- yl)amino]-5-fluoro-pyrimidin-4-yl]-3- methyl-indol-5-yl]prop-2-enamide | 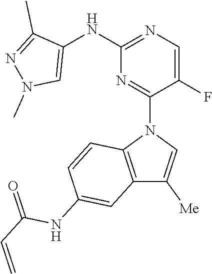 [SMILES: C=CC(=O)Nc1ccc2c(c1)c(C)cn2-c1nc(Nc2cn(C)nc2C)ncc1F] | LCMS m/z = 406 (M + 1); 1H NMR (500 MHz, DMSO-d6) δ: 9.91 (s, 1H), 8.52 (s, 1H), 8.43 (d, J = 4.6 Hz, 1H), 8.18 (d, J = 9.2 Hz, 1H), 8.00 (s, 1H), 7.71 (s, 1H), 7.64 (s, 1H), 7.41 (d, J = 9.2 Hz, 1H), 6.47 (dd, J = 17.1, 10.1 Hz, 1H), 6.27 (d, J = 17.1 Hz, 1H), 5.73 (d, J = 10.4 Hz, 1H), 3.78 (s, 3H), 2.29 (s, 3H), 2.10 (s, 3H) |
| Example 168 N-[1-methyl-3-[2-[[1-(2,2,2- trifluoroethyl)pyrazol-4- yl]amino]pyrimidin-4-yl]indol-6- yl]prop-2-enamide | 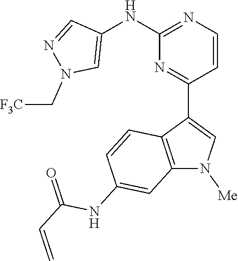 [SMILES: C=CC(=O)Nc1ccc2c(-c3ccnc(Nc4cnn(CC(F)(F)F)c4)n3)cn(C)c2c1] | LCMS m/z = 442 (M + 1); 1H NMR (DMSO-d6, 500 MHz) : 9.93 (s, 1H), 8.99 (s, 1H), 8.40 (d, J = 8.5 Hz, 1H), 8.31 (d, J = 5.2 Hz, 1H), 8.11 (s, 1H), 8.08 (s, 1H), 8.06 (s, 1H), 7.73 (s, 1H), 7.33 (dd, J = 8.7, 1.7 Hz, 1H), 7.08 (d, J = 5.5 Hz, 1H), 6.50 (dd, J = 16.9, 10.2 Hz, 1H), 6.29 (dd, J = 17.1, 1.8 Hz, 1H), 5.73 (dd, J = 10.2, 1.7 Hz, 1H), 5.03 (d, J = 9.2 Hz, 2H), 3.84 (s, 3H) |
| Example 169 N-[3-[2-[[1-(2,2-difluoroethyl)-3- methyl-pyrazol-4-yl]amino]-5-fluoro- pyrimidin-4-yl]-1-methyl-indol-6- yl]prop-2-enamide | 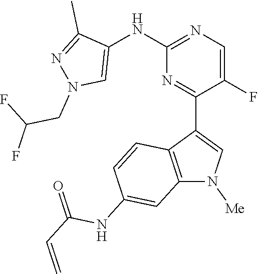 [SMILES: C=CC(=O)Nc1ccc2c(-c3nc(Nc4cn(CC(F)F)nc4C)ncc3F)cn(C)c2c1] | LCMS m/z = 456 (M + 1); 1H NMR (500 MHz, DMSO-d6: 9.93 (br.s, 1H), 8.37 (d, J = 8.5 Hz, 1H), 8.26 (d, J = 3.7 Hz, 1H), 8.24 (br.s, 1H), 8.07 (s, 1H), 8.05 (d, J = 2.7 Hz, 1H), 7.85 (s, 1H), 7.25 (dd, J = 8.5, 1.4 Hz, 1H), 6.49 (dd, J = 17.8, 10.4 Hz , 1H), 6.31 (tt, J = 55.1, 3.8 Hz, 1H), 6.28 (dd, J = 16.9, 1.7 Hz, 1H), 5.72 (dd, J = 10.4, 1.8 Hz, 1H), 4.49 (dt, J = 14.7, 3.9 Hz, 2H), 3.85 (s, 3H), 2.15 (s, 3H) |
| Example 170 N-[3-[2-[[1-(2,2-difluoroethyl)-3- methyl-pyrazol-4-yl]amino]pyrimidin- 4-yl]-1-methyl-indol-6-yl]prop-2- enamide | 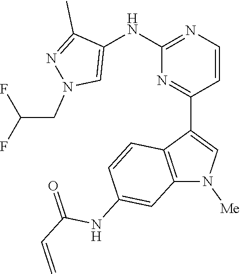 [SMILES: C=CC(=O)Nc1ccc2c(-c3ccnc(Nc4cn(CC(F)F)nc4C)n3)cn(C)c2c1] | LCMS m/z = 438 (M + 1); 1H NMR (300 MHz, DMSO-d6) δ: 10.21 (s, 1H), 8.54 (s, 1H), 8.27 - 8.22 (m, 1H), 8.21 (s, 1H), 8.14 (s, 1H), 7.97 (s, 1H), 7.22 (br s, 1H), 7.06 (d, J = 5.5 Hz, 1H), 6.53 - 6.45 (m, 1H), 6.37 - 6.24 (m, 2H), 5.77 - 5.74 (m, 1H), 4.53 (dt, J = 3.5, 14.9 Hz, 2H), 3.82 (s, 3H), 2.17 (s, 3H) |
| Example 171 (E)-4-(dimethylamino)-N-[1-[2-[(1- ethyl-3-methyl-pyrazol-4-yl)amino]- 5-fluoro-pyrimidin-4-yl]-3-methyl- pyrrolo[2,3-b]pyridin-5-yl]but-2- enamide | 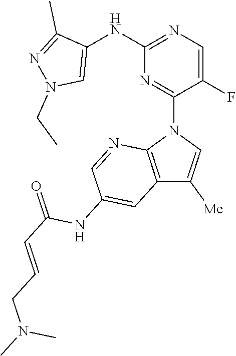 [SMILES: CCn1cc(Nc2ncc(F)c(-n3cc(C)c4cc(NC(=O)/C=C/CN(C)C)cnc43)n2)c(C)n1] | LCMS m/z = 478 (M + 1); 1H NMR (500 MHz, DMSO-d6) δ: 10.02 (s, 1H), 8.68 (s, 1H), 8.54 (d, J = 2.1 Hz, 1H), 8.48 (s, 1H), 8.39 (s, 1H), 7.99 (s, 1H), 7.60 (s, 1H), 6.80 (dt, J = 15.4, 5.9 Hz, 1H), 6.31 (d, J = 15.6 Hz, 1H), 4.02 (q, J = 7.3 Hz, 2H), 3.10 (d, J = 5.8 Hz, 2H), 2.32 (s, 3H), 2.23 (s, 6H), 2.15 (s, 3H), 1.36 (t, J = 7.2 Hz, 3H) |
| Example 172 N-[3-[2-[(1,3-dimethylpyrazol-4- yl)amino]pyrimidin-4-yl]-1-methyl- indol-6-yl]prop-2-enamide | 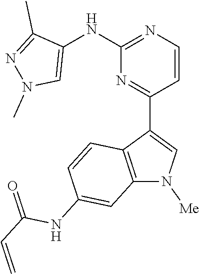 [SMILES: C=CC(=O)Nc1ccc2c(-c3ccnc(Nc4cn(C)nc4C)n3)cn(C)c2c1] | LCMS m/z = 388 (M + 1); 1H NMR (400 MHz, DMSO-d6) δ: 9.95 - 9.87 (m, 1H), 8.33 - 8.26 (m, 1H), 8.25 - 8.18 (m, 1H), 8.10 - 8.08 (m, 1H), 8.08 (s, 2H), 7.76 (s, 1H), 7.26 (br s, 1H), 7.05 - 6.97 (m, 1H), 6.53 - 6.43 (m, 1H), 6.31 - 6.22 (m, 1H), 5.74 - 5.69 (m, 1H), 3.87 - 3.71 (m, 6H), 2.13 (s, 3H) |
| Example 173 N-[3-[2-[(1-ethyl-3-methyl-pyrazol-4- yl)amino]pyrimidin-4-yl]-1-methyl- indol-6-yl]prop-2-enamide | 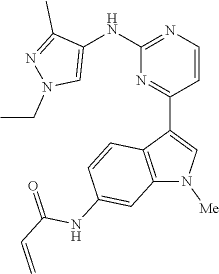 [SMILES: C=CC(=O)Nc1ccc2c(-c3ccnc(Nc4cn(CC)nc4C)n3)cn(C)c2c1] | LCMS m/z = 402 (M + 1); 1H NMR (400 MHz, DMSO-d6) δ: 9.91 (s, 1H), 8.27 (br d, J = 8.7 Hz, 1H), 8.21 (br d, J = 4.5 Hz, 1H), 8.07 (s, 1H), 8.03 (s, 1H), 7.80 (s, 1H), 7.24 (d, J = 8.8 Hz, 1H), 7.01 (d, J = 4.9 Hz, 1H), 6.48 (br dd, J = 16.9, 10.0 Hz, 1H), 6.27 (br d, J = 16.6 Hz, 1H), 5.71 (br d, J = 9.7 Hz, 1H), 4.06 (q, J = 7.3 Hz, 2H), 3.81 (s, 4H), 2.13 (s, 3H), 1.41 (br t, J = 7.0 Hz, 3H) |
| Example 174 N-[1-[5-fluoro-2-[[3-methyl-1-(2- morpholinoethyl)pyrazol-4- yl]amino]pyrimidin-4-yl]-3-methyl- indol-5-yl]prop-2-enamide N-[1-[5-fluoro-2-[[5-methyl-1-(2- morpholinoethyl)pyrazol-4- yl]amino]pyrimidin-4-yl]-3-methyl- indol-5-yl]prop-2-enamide | 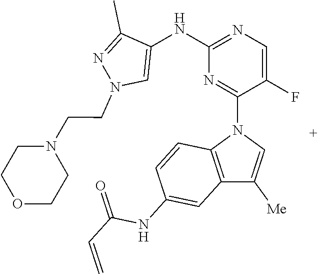 [SMILES: C=CC(=O)Nc1ccc2c(c1)c(C)cn2-c1nc(Nc2cn(CCN3CCOCC3)nc2C)ncc1F] | LCMS m/z = 505 (M + 1). |
| | 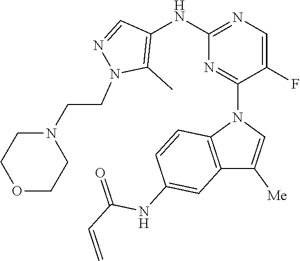 [SMILES: C=CC(=O)Nc1ccc2c(c1)c(C)cn2-c1nc(Nc2cnn(CCN3CCOCC3)c2C)ncc1F] | |
| Example 175 N-[1-[5-fluoro-2-[[1-[(1- hydroxycyclopropyl)methyl]pyrazol- 4-yl]amino]pyrimidin-4-yl]-3-methyl- indol-5-yl]prop-2-enamide | 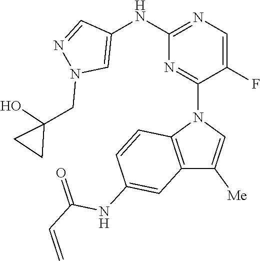 [SMILES: C=CC(=O)Nc1ccc2c(c1)c(C)cn2-c1nc(Nc2cnn(CC3(O)CC3)c2)ncc1F] | LCMS m/z = 448 (M + 1); 1H NMR (500 MHz, DMSO-d6) δ: 10.22 (s, 1H), 9.60 (s, 1H), 8.55 (d, J = 4.5 Hz, 2H), 8.08 (s, 1H), 7.97 (s, 1H), 7.68 (s, 1H), 7.53 (s, 1H), 7.49 (br.s, 1H), 6.48 (dd, J = 17.0, 10.0 Hz , 1H), 6.28 (dd, J = 17.0, 2.0 Hz, 1H), 5.76 (dd, J = 10.0, 2.0 Hz, 1H), 4.13 (s, 2H), 2.63 (s, 1H), 2.29 (s, 3H), 0.63 (s, 4H) |
| Example 176 N-[1-[5-fluoro-2-[[1-(2- hydroxyethyl)-3-methyl-pyrazol-4- yl]amino]pyrimidin-4-yl]-3-methyl- indol-5-yl]prop-2-enamide | 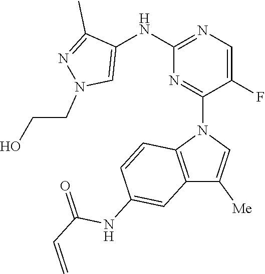 [SMILES: C=CC(=O)Nc1ccc2c(c1)c(C)cn2-c1nc(Nc2cn(CCO)nc2C)ncc1F] | LCMS m/z = 436 (M + 1); 1H NMR (500 MHz, DMSO-d6) δ: 9.91 (br s, 1H), 8.53 (br s, 1H), 8.43 (br d, J = 4.9 Hz, 1H), 8.18 (br d, J = 8.5 Hz, 1H), 8.00 (br s, 1H), 7.76 (s, 1H), 7.64 (br s, 1H), 7.40 (br d, J = 9.2 Hz, 1H), 6.47 (br dd, J = 16.9, 10.2 Hz, 1H), 6.27 (br d, J = 16.8 Hz, 1H), 5.72 (br d, J = 10.7 Hz, 1H), 4.58 (br s, 1H), 4.09 - 4.05 (m, 2H), 3.77 (br d, J = 5.5 Hz, 2H), 2.29 (s, 3H), 2.11 (s, 3H) |
| Example 177 N-[3-[2-[(1-ethyl-3-methyl-pyrazol-4- yl)amino]-5-fluoro-pyrimidin-4-yl]-1- methyl-indol-6-yl]prop-2-enamide | 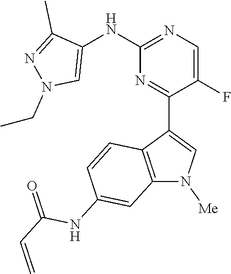 [SMILES: C=CC(=O)Nc1ccc2c(-c3nc(Nc4cn(CC)nc4C)ncc3F)cn(C)c2c1] | LCMS m/z = 420 (M + 1); 1H NMR (500 MHz, DMSO-d6): 9.94 (br.s, 1H), 8.33 (d, J = 8.1 Hz, 1H), 8.23 (d, J = 3.6 Hz, 1H), 8.14 (br.s, 1H), 8.06 (d, J = 1.2 Hz, 1H), 8.03 (d, J = 2.7 Hz, 1H), 7.73 (s, 1H), 7.22 (dd, J = 8.6, 1.8 Hz, 1H), 6.47 (dd, J = 16.8, 10.2 Hz, 1H), 6.26 (dd, J = 17.0, 1.8 Hz, 1H), 5.71 (dd, J = 10.0, 1.6 Hz, 1H), 4.05(q, J = 7.1 Hz, 2H), 3.83 (s, 3H), 2.09 (s, 3H), 1.40 (t, J = 7.3 Hz, 3H) |
| Example 178 N-[3-[2-[(1-cyclopropylpyrazol-4- yl)amino]pyrimidin-4-yl]-1-methyl- indol-6-yl]prop-2-enamide | 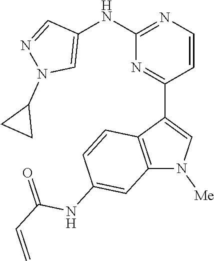 [SMILES: C=CC(=O)Nc1ccc2c(-c3ccnc(Nc4cnn(C5CC5)c4)n3)cn(C)c2c1] | LCMS m/z = 400 (M + 1); 1H NMR (500 MHz, DMSO-d6) δ: 10.23 (s, 1H), 9.20 (s, 1H), 8.52 (bs, 1H), 8.28 (d, J = 5.2 Hz, 1H), 8.21 (s, 1H), 8.14 (s, 1H), 8.00 (s, 1H), 7.50 (s, 1H), 7.29 (dd, J = 1.4, 8.7 Hz, 1H), 7.05 (d, J = 5.5 Hz, 1H), 6.49 (dd, J = 10.2, 16.9 Hz, 1H), 6.28 (dd, J = 1.8, 16.8 Hz, 1H), 5.78 - 5.73 (m, 1H), 3.83 (s, 3H), 3.69 (tt, J = 3.9, 7.3 Hz, 1H), 1.06 - 0.92 (m, 4H). |
| Example 179 (E)-4-(dimethylamino)-N-[1-[2-[(1,3- dimethylpyrazol-4-yl)amino]-5- fluoro-pyrimidin-4-yl]-3-methyl- pyrrolo[2,3-b]pyridin-5-yl]but-2- enamide | 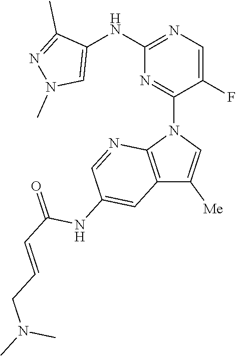 [SMILES: Cc1nn(C)cc1Nc1ncc(F)c(-n2cc(C)c3cc(NC(=O)/C=C/CN(C)C)cnc32)n1] | LCMS m/z = 464 (M + 1); 1H NMR (500 MHz, DMSO-d6) δ: 10.03 (br s, 1H), 8.69 (br s, 1H), 8.54 (d, J = 3.1 Hz, 1H), 8.49 (d, J = 2.1 Hz, 1H), 8.39 (d, J = 2.1 Hz, 1H), 7.97 (s, 1H), 7.60 (s, 1H), 6.80 (dt, J = 15.4, 5.9 Hz, 1H), 6.31 (d, J = 15.6 Hz, 1H), 3.74 (s, 3H), 3.10 (d, J = 5.8 Hz, 2H), 2.32 (s, 3H), 2.23 (s, 6H), 2.14 (s, 3H) |
| Example 180 (E)-N-[1-[5-chloro-2-[(1,3- dimethylpyrazol-4- yl)amino]pyrimidin-4-yl]-3-methyl- pyrrolo[2,3-b]pyridin-5-yl]-4- (dimethylamino)but-2-enamide | 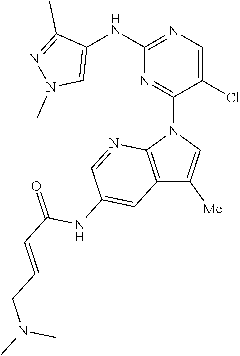 [SMILES: Cc1nn(C)cc1Nc1ncc(Cl)c(-n2cc(C)c3cc(NC(=O)/C=C/CN(C)C)cnc32)n1] | LCMS m/z = 480 (M + 1); 1H NMR (500 MHz, DMSO-d6) δ: 9.99 (s, 1H), 8.95 (s, 1H), 8.56 (s, 1H), 8.45 (d, J = 2.1 Hz, 1H), 8.37 (d, J = 2.1 Hz, 1H), 7.85 (s, 1H), 7.53 (s, 1H), 6.80 (dt, J = 15.4, 5.9 Hz, 1H), 6.30 (d, J = 15.6 Hz, 1H), 3.71 (s, 3H), 3.10 (d, J = 6.0 Hz, 5H), 2.31 (d, J = 0.9 Hz, 3H), 2.23 (s, 6H), 2.13 (s, 3H) |
| Example 181 N-[1-[5-fluoro-2-[[1-(2-hydroxy-2- methyl-propyl)pyrazol-4- yl]amino]pyrimidin-4-yl]-3-methyl- indol-5-yl]prop-2-enamide | 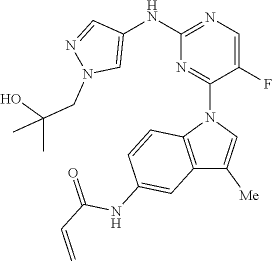 [SMILES: C=CC(=O)Nc1ccc2c(c1)c(C)cn2-c1nc(Nc2cnn(CC(C)(C)O)c2)ncc1F] | LCMS m/z = 450 (M + 1); 1H NMR (500 MHz, DMSO-d6) δ: 9.94 (s, 1H), 9.28 (s, 1H), 8.50 (d, J = 4.6 Hz, 1H), 8.31 (d, J = 8.2 Hz, 1H), 8.01 (s, 1H), 7.88 (s, 1H), 7.64 (s, 1H), 7.57 (s, 1H), 7.49 (d, J = 8.5 Hz, 1H), 6.46 - 6.44 (m, 1H), 6.32 - 6.24 (m, 1H), 5.76 - 5.70 (m, 1H), 4.41 (s, 1H), 3.99 (s, 2H), 2.30 (s, 3H), 1.10 (s, 6H) |
| Example 182 N-[1-[5-fluoro-2-[[1-(2-hydroxy-2- methyl-propyl)-3-methyl-pyrazol-4- yl]amino]pyrimidin-4-yl]-3-methyl- indol-5-yl]prop-2-enamide | 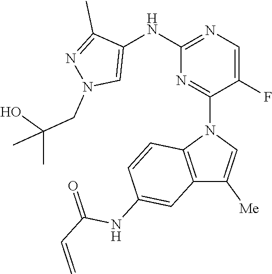 [SMILES: C=CC(=O)Nc1ccc2c(c1)c(C)cn2-c1nc(Nc2cn(CC(C)(C)O)nc2C)ncc1F] | LCMS m/z = 464 (M + 1); 1H NMR (600 MHz, DMSO-d6) δ: 9.85 (s, 1H), 8.44 (s, 1H), 8.38 (d, J = 4.8 Hz, 1H), 8.12 (br d, J = 8.1 Hz, 1H), 7.96 (d, J = 2.0 Hz, 1H), 7.61 (m, 1H), 7.50 (s, 1H), 7.37 (s, 1H), 6.46 (dd, J = 17.1, 10.3 Hz, 1H), 6.26 (dd, J = 17.1, 1.8 Hz, 1H), 5.70 (dd, J = 10.2, 1.9 Hz, 1H), 4.42 (s, 1H), 3.97 (s, 2H), 2.26 (d, J = 1.3 Hz, 3H), 2.19 (s, 3H), 1.14 (s, 6H) |
| Example 183 N-[1-[5-fluoro-2-[[1-(2-hydroxy-2- methyl-propyl)-5-methyl-pyrazol-4- yl]amino]pyrimidin-4-yl]-3-methyl- indol-5-yl]prop-2-enamide | 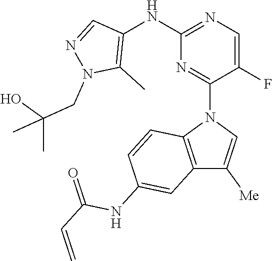 [SMILES: C=CC(=O)Nc1ccc2c(c1)c(C)cn2-c1nc(Nc2cnn(CC(C)(C)O)c2C)ncc1F] | LCMS m/z = 464 (M + 1); 1H NMR (600 MHz, DMSO-d6) δ: 9.86 (s, 1H), 8.49 (s, 1H), 8.41 (d, J = 4.8 Hz, 1H), 8.15 (br d, J = 8.3 Hz, 1H), 7.96 (d, J = 1.8 Hz, 1H), 7.74 (s, 1H), 7.62 (dd, J = 2.1, 1.2 Hz, 1H), 7.33-7.43 (m, 1H), 6.46 (dd, J = 17.0, 10.2 Hz, 1H), 6.26 (dd, J = 17.0, 1.9 Hz, 1H), 5.70 (dd, J = 10.3, 2.0 Hz, 1H), 4.36 (s, 1H), 3.93 (s, 2H), 2.27 (d, J = 1.1 Hz, 3H), 2.10 (s, 3H), 1.10 (s, 6H) |
| Example 184 N-[1-methyl-3-[2-[[1- (trifluoromethyl)pyrazol-4- yl]amino]pyrimidin-4-yl]indol-6- yl]prop-2-enamide | 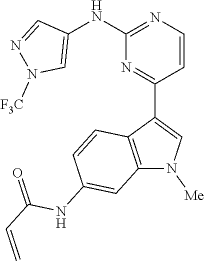 [SMILES: C=CC(=O)Nc1ccc2c(-c3ccnc(Nc4cnn(C(F)(F)F)c4)n3)cn(C)c2c1] | LCMS m/z = 428 (M + 1); 1H NMR (DMSO-d6, 600 MHz) δ: 9.94 (s, 1H), 9.31 (s, 1H), 8.44 (s, 1H), 8.38 (d, J = 8.8 Hz, 1H), 8.35 (d, J = 5.5 Hz, 1H), 8.13 (s, 1H), 8.03-8.08 (m, 2H), 7.31 (dd, J = 8.6, 1.8 Hz, 1H), 7.15 (d, J = 5.3 Hz, 1H), 6.48 (dd, J = 17.1, 10.3 Hz, 1H), 6.27 (dd, J = 17.1, 1.8 Hz, 1H), 5.71 (dd, J = 10.2, 1.9 Hz, 1H), 3.83 (s, 3H) |
| Example 185 N-[1-[5-fluoro-2-[[1-[(2R)-2- hydroxypropyl]pyrazol-4- yl]amino]pyrimidin-4-yl]-3-methyl- indol-5-yl]prop-2-enamide | 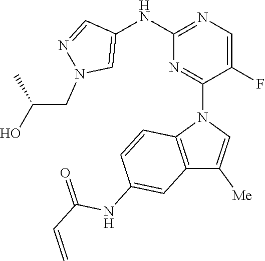 [SMILES: C=CC(=O)Nc1ccc2c(c1)c(C)cn2-c1nc(Nc2cnn(C[C@@H](C)O)c2)ncc1F] | LCMS m/z = 436 (M + 1); 1H NMR (500 MHz, DMSO-d6) δ: 9.93 (s, 1H), 9.27 (s, 1H), 8.49 (d, J = 4.6 Hz, 1H), 8.31 (d, J = 8.9 Hz, 1H), 8.01 (d, J = 1.5 Hz, 1H), 7.86 (s, 1H), 7.64 (s, 1H), 7.55 (s, 1H), 7.50 (dd, J = 8.9, 1.8 Hz, 1H), 6.48 (dd, J = 17.1, 10.4 Hz, 1H), 6.28 (dd, J = 16.9, 2.0 Hz, 1H), 5.73 (dd, J = 10.1, 1.8 Hz, 1H), 4.60 (d, J = 4.0 Hz, 1H), 3.94-4.03 (m, 3H), 2.30 (d, J = 0.9 Hz, 3H), 1.07 (d, J = 5.8 Hz, 3H) |
| Example 186 N-[1-[5-fluoro-2-[[1-[(2S)-2- hydroxypropyl]pyrazol-4- yl]amino]pyrimidin-4-yl]-3-methyl- indol-5-yl]prop-2-enamide | 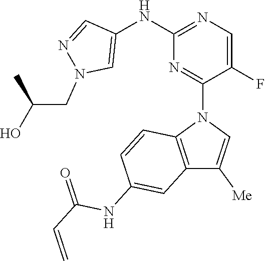 [SMILES: C=CC(=O)Nc1ccc2c(c1)c(C)cn2-c1nc(Nc2cnn(C[C@H](C)O)c2)ncc1F] | LCMS m/z = 436 (M + 1); 1H NMR (500 MHz, DMSO-d6) δ: 9.94 (s, 1H), 9.27 (s, 1H), 8.50 (d, J = 4.9 Hz, 1H), 8.31 (d, J = 8.9 Hz, 1H), 8.01 (s, 1H), 7.86 (s, 1H), 7.64 (s, 1H), 7.55 (s, 1H), 7.49 (dd, J = 8.9, 1.8 Hz, 1H), 6.48 (dd, J = 16.9, 10.2 Hz, 1H), 6.28 (dd, J = 17.1, 1.8 Hz, 1H), 5.73 (dd, J = 10.4, 1.8 Hz, 1H), 4.60 (d, J = 3.1 Hz, 1H), 3.94-4.03 (m, 3H), 2.30 (s, 3H), 1.07 (d, J = 5.5 Hz, 3H) |
| Example 187 N-[1-[2-[(1,3-dimethylpyrazol-4- yl)amino]-5-methyl-pyrimidin-4-yl]- 3-methyl-indol-5-yl]prop-2-enamide | 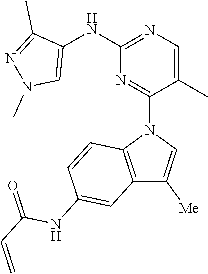 [SMILES: C=CC(=O)Nc1ccc2c(c1)c(C)cn2-c1nc(Nc2cn(C)nc2C)ncc1C] | LCMS m/z = 402 (M + 1); 1H NMR (500 MHz, DMSO-d6) δ: 10.12 (s, 1H), 8.73 (s, 1H), 8.37 (s, 1H), 8.04 (s, 1H), 7.76 (s, 1H), 7.71 (br.s, 1H), 7.54 (s, 1H), 7.36 (d, J = 9.0 Hz, 1H), 6.47 (dd, J = 17.0, 10.0 Hz, 1H), 6.27 (dd, J = 17.0, 2.0 Hz, 1H), 5.74 (dd, J = 10.0, 2.0 Hz, 1H), 3.69 (s, 3H), 2.28 (s, 3H), 2.19 (s, 3H), 2.11 (s, 3H) |
| Example 188 N-[3-[2-[[1-(difluoromethyl)pyrazol- 4-yl]amino]pyrimidin-4-yl]-1-methyl- indol-6-yl]prop-2-enamide | 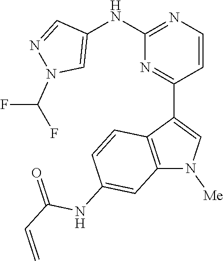 [SMILES: C=CC(=O)Nc1ccc2c(-c3ccnc(Nc4cnn(C(F)F)c4)n3)cn(C)c2c1] | LCMS m/z = 410 (M + 1); 1H NMR (DMSO-d6, 600 MHz) : 9.95 (s, 1H), 9.20 (s, 1H), 8.42 (d, J = 8.4 Hz, 1H), 8.36 (s, 1H), 8.35 (d, J = 5.3 Hz, 1H), 8.14 (s, 1H), 8.08 (d, J = 1.3 Hz, 1H), 7.93 (s, 1H), 7.69 (t, J = 61.3 Hz, 1H), 7.34 (dd, J = 8.7, 1.7 Hz, 1H), 7.14 (d, J = 5.3 Hz, 1H), 6.50 (dd, J = 16.9, 10.3 Hz, 1H), 6.29 (dd, J = 17.1, 1.8 Hz, 1H), 5.74 (dd, J = 10.3, 1.8 Hz, 1H), 3.85 (s, 3H) |
| Example 189 N-[3-[2-[(1-cyclobutylpyrazol-4- yl)amino]pyrimidin-4-yl]-1-methyl- indol-6-yl]prop-2-enamide | 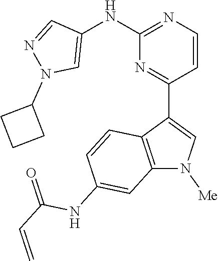 [SMILES: C=CC(=O)Nc1ccc2c(-c3ccnc(Nc4cnn(C5CCC5)c4)n3)cn(C)c2c1] | LCMS m/z = 414 (M + 1); 1H NMR (DMSO-d6, 500 MHz) : 10.23 (s, 1H), 9.20 (br s, 1H), 8.53 (br s, 1H), 8.28 (d, J = 5.2 Hz, 1H), 8.21 (s, 1H), 8.14 (s, 1H), 8.02 (s, 1H), 7.55 (s, 1H), 7.28 (br d, J = 8.5 Hz, 1H), 7.05 (d, J = 5.2 Hz, 1H), 6.49 (dd, J = 16.9, 10.2 Hz, 1H), 6.28 (br d, J = 17.1 Hz, 1H), 5.76 (br d, J = 11.6 Hz, 1H), 4.77-4.84 (m, 1H), 3.83 (s, 3H), 2.37-2.47 (m, 4H), 1.73- 1.84 (m, 2H) |
| Example 190 N-[1-methyl-3-[2-[[1-(oxetan-3- yl)pyrazol-4-yl]amino]pyrimidin-4- yl]indol-6-yl]prop-2-enamide | 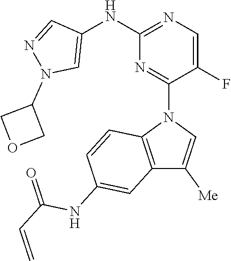 [SMILES: C=CC(=O)Nc1ccc2c(c1)c(C)cn2-c1nc(Nc2cnn(C3COC3)c2)ncc1F] | LCMS m/z = 334 (M + 1); 1H NMR (500 MHz, DMSO-d6) δ: 9.94 (s, 1H), 9.35 (s, 1H), 8.51 (d, J = 4.3 Hz, 1H), 8.30 (d, J = 7.9 Hz, 1H), 8.05 - 7.99 (m, 2H), 7.68 (s, 1H), 7.64 (bs, 1H), 7.53 - 7.46 (m, 1H), 6.48 (dd, J = 10.2, 16.9 Hz, 1H), 6.31 - 6.25 (m, 1H), 5.76 - 5.71 (m, 1H), 5.55 - 5.47 (m, 1H), 4.96 - 4.88 (m, 4H), 2.30 (s, 3H). |
| Example 191 (E)-4-(dimethylamino)-N-[1-[5- fluoro-2-[[1-[(2R)-2- hydroxypropyl]pyrazol-4- yl]amino]pyrimidin-4-yl]-3-methyl- pyrrolo[2,3-b]pyridin-5-yl]but-2- enamide | 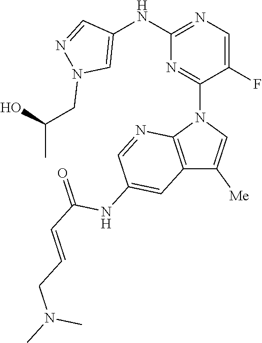 [SMILES: Cc1cn(-c2nc(Nc3cnn(C[C@@H](C)O)c3)ncc2F)c2ncc(NC(=O)/C=C/CN(C)C)cc12] | LCMS m/z = 494 (M + 1); 1H NMR (500 MHz, DMSO-d6) δ: 10.04 (s, 1H), 9.38 (s, 1H), 8.58 (d, J = 3.1 Hz, 1H), 8.50 (d, J = 2.1 Hz, 1H), 8.40 (d, J = 2.1 Hz, 1H), 8.01 (s, 1H), 7.64 (s, 1H), 7.60 (s, 1H), 6.81 (dt, J = 15.4, 5.9 Hz, 1H), 6.31 (d, J = 15.6 Hz, 1H), 4.58 (d, J = 4.3 Hz, 1H), 3.93-4.02 (m, 3H), 3.11 (br d, J = 5.8 Hz, 2H), 2.33 (s, 3H), 2.24 (s, 6H), 1.05 (d, J = 5.8 Hz, 3H) |
| Example 192 N-[1-[5-fluoro-2-[[1-(2- hydroxyethyl)-5-methyl-pyrazol-4- yl]amino]pyrimidin-4-yl]-3-methyl- indol-5-yl]prop-2-enamide | 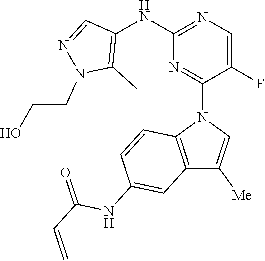 [SMILES: C=CC(=O)Nc1ccc2c(c1)c(C)cn2-c1nc(Nc2cnn(CCO)c2C)ncc1F] | LCMS m/z = 436 (M + 1); 1H NMR (400 MHz, DMSO-d6) δ: 9.88 (s, 1H), 8.42 (s, 1H), 8.38 (d, J = 4.9 Hz, 1H), 8.13 - 8.09 (m, 1H), 7.97 (d, J = 2.0 Hz, 1H), 7.66 - 7.59 (m, 1H), 7.47 (s, 1H), 7.38 (dd, J = 9.0, 2.1 Hz, 1H), 6.53 - 6.42 (m, 1H), 6.29 - 6.21 (m, 1H), 5.71 (dd, J = 10.2, 2.0 Hz, 1H), 4.53 (s, 1H), 4.10 (t, J = 6.1 Hz, 2H), 3.78 (q, J = 6.0 Hz, 2H), 2.27 (d, J = 1.2 Hz, 3H), 2.18 (s, 3H) |
| Example 193 N-[1-[2-[[1-(2-ethoxyethyl)pyrazol-4- ylamino]-5-fluoro-pyrimidin-4-yl]-3- methyl-indol-5-yl]prop-2-enamide | 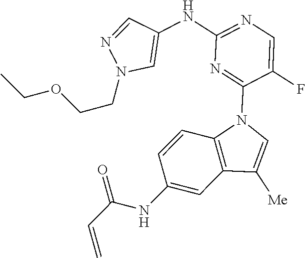 [SMILES: C=CC(=O)Nc1ccc2c(c1)c(C)cn2-c1nc(Nc2cnn(CCOCC)c2)ncc1F] | LCMS m/z = 450 (M + 1); (d, J = 4.4 Hz, 2H), 8.07 (d, J = 2.0 Hz, 1H), 7.91 (br s, 1H), 7.67 (br s, 1H), 7.55 (d, J = 0.7 Hz, 1H), 7.42-7.52 (m, 1H), 6.48 (dd, J = 17.0, 10.2 Hz, 1H), 6.28 (dd, J = 17.0, 1.9 Hz, 1H), 5.75-5.78 (m, 1H), 4.20 (t, J = 5.5 Hz, 2H), 3.70 (t, J = 5.5 Hz, 2H), 3.37-3.45 (m, 2H), 2.29 (d, J = 1.3 Hz, 3H), 1.05 (br s, 3H) |
| Example 194 N-[1-[5-fluoro-2-[[1-[(2R)-2- hydroxypropyl]-3-methyl-pyrazol-4- yl]amino]pyrimidin-4-yl]-3-methyl- indol-5-yl]prop-2-enamide N-[1-[5-fluoro-2-[[1-[(2R)-2- hydroxypropyl]-5-methyl-pyrazol-4- yl]amino]pyrimidin-4-yl]-3-methyl- indol-5-yl]prop-2-enamide | 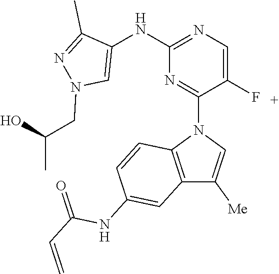 [SMILES: C=CC(=O)Nc1ccc2c(c1)c(C)cn2-c1nc(Nc2cn(C[C@@H](C)O)nc2C)ncc1F] | LCMS m/z = 450 (M + 1). |
| | 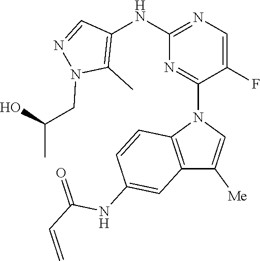 [SMILES: C=CC(=O)Nc1ccc2c(c1)c(C)cn2-c1nc(Nc2cnn(C[C@@H](C)O)c2C)ncc1F] | |
| Example 195 N-[1-[5-fluoro-2-[[1-[(2S)-2- hydroxypropyl]-3-methyl-pyrazol-4- yl]amino]pyrimidin-4-yl]-3-methyl- indol-5-yl]prop-2-enamide N-[1-[5-fluoro-2-[[1-[(2S)-2- hydroxypropyl]-5-methyl-pyrazol-4- yl]amino]pyrimidin-4-yl]-3-methyl- indol-5-yl]prop-2-enamide | 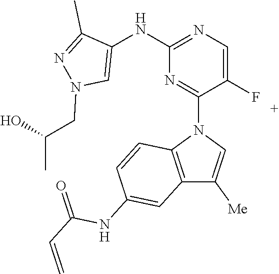 [SMILES: C=CC(=O)Nc1ccc2c(c1)c(C)cn2-c1nc(Nc2cn(C[C@H](C)O)nc2C)ncc1F] | LCMS m/z = 450 (M + 1); |
| | 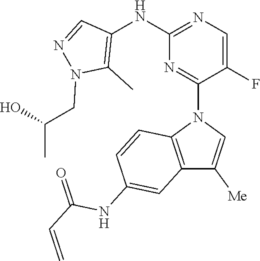 [SMILES: C=CC(=O)Nc1ccc2c(c1)c(C)cn2-c1nc(Nc2cnn(C[C@H](C)O)c2C)ncc1F] | |
| Example 196 (E)-4-(dimethylamino)-N-[1-[5- fluoro-2-[[1-[(2S)-2- hydroxypropyl]pyrazol-4- yl]amino]pyrimidin-4-yl]-3-methyl- pyrrolo[2,3-b]pyridin-5-yl]but-2- enamide | 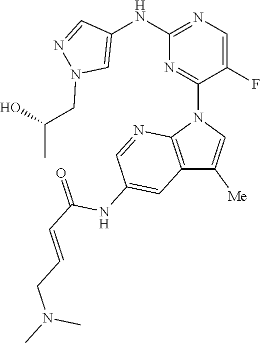 [SMILES: Cc1cn(-c2nc(Nc3cnn(C[C@H](C)O)c3)ncc2F)c2ncc(NC(=O)/C=C/CN(C)C)cc12] | LCMS m/z = 494 (M + 1); 1H NMR (300 MHz, DMSO-d6) δ: 10.31 (s, 1H), 9.70 (s, 1H), 8.63 (d, J = 3.0 Hz, 1H), 8.46 (s, 2H), 8.04 (br s, 1H), 7.68 (s, 1H), 7.56 (s, 1H), 6.77 (dt, J = 15.4, 5.8 Hz, 1H), 6.30 (br d, J = 15.3 Hz, 1H), 4.86 (d, J = 4.4 Hz, 1H), 3.94 (s, 3H), 3.07 (br d, J = 5.2 Hz, 2H), 2.31 (s, 3H), 2.18 (s, 6H), 1.00 (d, J = 5.6 Hz, 3H) |
| Example 197 (E)-N-[1-[5-fluoro-2-[[1-[(2R)-2- hydroxypropyl]pyrazol-4- yl]amino]pyrimidin-4-yl]-3-methyl- pyrrolo[2,3-b]pyridin-5-yl]-4- morpholino-but-2-enamide | 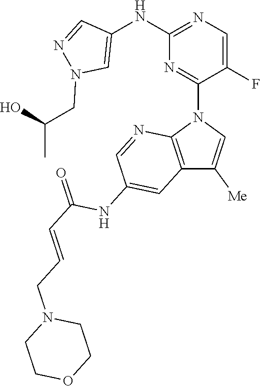 [SMILES: Cc1cn(-c2nc(Nc3cnn(C[C@@H](C)O)c3)ncc2F)c2ncc(NC(=O)/C=C/CN3CCOCC3)cc12] | LCMS m/z = 536 (M + 1); 1H NMR (500 MHz, DMSO-d6) δ: 10.32 (s, 1H), 9.70 (s, 1H), 8.64 (d, J = 2.7 Hz, 1H), 8.47 (s, 2H), 8.05 (br s, 1H), 7.70 (br s, 1H), 7.57 (br s, 1H), 6.77 (dt, J = 15.3, 5.8 Hz, 1H), 6.33 (d, J = 15.6 Hz, 1H), 4.86 (d, J = 4.6 Hz, 1H), 3.91-3.98 (m, 3H), 3.61 (t, J = 4.4 Hz, 4H), 3.15 (d, J = 5.8 Hz, 2H), 2.41 (br s, 4H), 2.32 (s, 3H), 1.01 (d, J = 5.5 Hz, 3H) |
| Example 198 (E)-N-[1-[5-fluoro-2-[[1-[(2S)-2- hydroxypropyl]pyrazol-4- yl]amino]pyrimidin-4-yl]-3-methyl- pyrrolo[2,3-b]pyridin-5-yl]-4- morpholino-but-2-enamide | 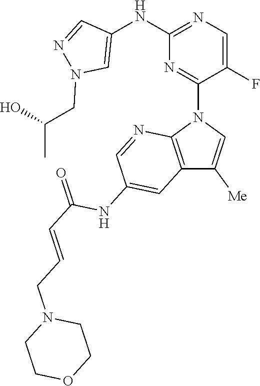 [SMILES: Cc1cn(-c2nc(Nc3cnn(C[C@H](C)O)c3)ncc2F)c2ncc(NC(=O)/C=C/CN3CCOCC3)cc12] | LCMS m/z = 536 (M + 1); 1H NMR (500 MHz, DMSO-d6) δ: 10.32 (s, 1H), 9.70 (s, 1H), 8.64 (d, J = 2.7 Hz, 1H), 8.47 (s, 2H), 8.04 (br s, 1H), 7.70 (br s, 1H), 7.57 (br s, 1H), 6.77 (dt, J = 15.3, 5.9 Hz, 1H), 6.33 (d, J = 15.6 Hz, 1H), 4.87 (d, J = 4.3 Hz, 1H), 3.90-4.00 (m, 3H), 3.61 (t, J = 4.4 Hz, 4H), 3.15 (d, J = 6.4 Hz, 2H), 2.41 (br s, 4H), 2.32 (s, 3H), 1.01 (d, J = 5.5 Hz, 3H) |
| Example 199 (E)-4-(dimethylamino)-N-[1-[2-[(1,5- dimethylpyrazol-4-yl)amino]-5- fluoro-pyrimidin-4-yl]-3-methyl- pyrrolo[2,3-b]pyridin-5-yl]but-2- enamide | 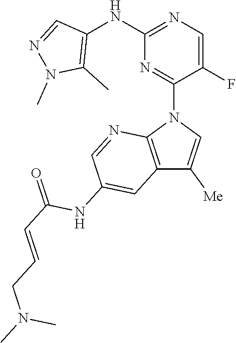 [SMILES: Cc1cn(-c2nc(Nc3cnn(C)c3C)ncc2F)c2ncc(NC(=O)/C=C/CN(C)C)cc12] | LCMS m/z = 464 (M + 1); 1H NMR (500 MHz, DMSO-d6) δ: 9.99 (s, 1H), 8.61 (s, 1H), 8.49 (d, J = 2.7 Hz, 1H), 8.42 (d, J = 2.4 Hz, 1H), 8.37 (d, J = 2.1 Hz, 1H), 7.54 (s, 1H), 7.54 (br s, 1H), 6.78 (dt, J = 15.6, 6.0 Hz, 1H), 6.28 (d, J = 15.3 Hz, 1H), 3.69 (s, 3H), 3.08 (br d, J = 5.8 Hz, 2H), 2.30 (s, 3H), 2.21 (s, 6H), 2.18 (s, 3H) |
| Example 200 (E)-4-(dimethylamino)-N-[1-[2-[(1,3- dimethylpyrazol-4-yl)amino]-5- methyl-pyrimidin-4-yl]-3-methyl- pyrrolo[2,3-b]pyridin-5-yl]but-2- enamide | 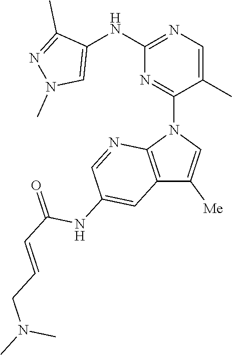 | LCMS m/z = 460 (M + 1); 1H NMR (500 MHz, DMSO-d6) δ: 10.25 (s, 1H), 8.82 (br s, 1H), 8.44 (d, J = 2.4 Hz, 1H), 8.41 (s, 1H), 8.39 (d, J = 2.3 Hz, 1H), 7.84 (s, 1H), 7.58 (s, 1H), 6.76 (dt, J = 15.4, 5.9 Hz, 1H), 6.30 (d, J = 15.6 Hz, 1H), 3.70 (s, 3H), 3.08 (d, J = 5.8 Hz, 2H), 2.31 (s, 3H), 2.19 (s, 6H), 2.10 (s, 3H), 2.07 (s, 3H) |
| Example 201 N-[3-[2-[(1-methylpyrazol-4- yl)amino]pyrimidin-4-yl]-1-methyl- indol-6-yl]prop-2-enamide | 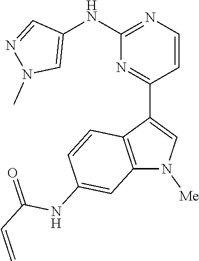 [SMILES: C=CC(=O)Nc1ccc2c(-c3ccnc(Nc4cnn(C)c4)n3)cn(C)c2c1] | LCMS m/z = 374 (M + 1); 1H NMR (400 MHz, DMSO-d6) δ: 10.22 (s, 1H), 9.19 (s, 1H), 8.50 (br s, 1H), 8.26 (d, J = 5.4 Hz, 1H), 8.21 (s, 1H), 8.13 (d, J = 1.5 Hz, 1H), 7.92 (s, 1H), 7.51 (s, 1H), 7.27 (dd, J = 8.7, 1.7 Hz, 1H), 7.04 (d, J = 5.4 Hz, 1H), 6.48 (dd, J = 16.9, 10.4 Hz, 1H), 6.27 (dd, J = 16.9, 2.0 Hz, 1H), 5.73-5.77 (m, 1H), 3.82 (br s, 6H) |
| Example 202 N-[1-[5-fluoro-2-[[1-(2- methoxyethyl)-3-methyl-pyrazol-4- yl]amino]pyrimidin-4-yl]-3-methyl- indol-5-yl]prop-2-enamide | 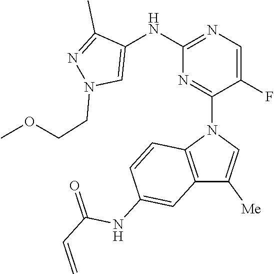 [SMILES: C=CC(=O)Nc1ccc2c(c1)c(C)cn2-c1nc(Nc2cn(CCOC)nc2C)ncc1F] | LCMS m/z = 450 (M + 1); 1H NMR (500 MHz, DMSO-d6) δ: 9.91 (s, 1H), 8.53 (s, 1H), 8.43 (d, J = 4.9 Hz, 1H), 8.16 (d, J = 8.5 Hz, 1H), 7.98 (s, 1H), 7.75 (s, 1H), 7.64 (s, 1H), 7.42 (d, J = 8.9 Hz, 1H), 6.47 (dd, J = 10.1, 17.1 Hz, 1H), 6.30 - 6.25 (m, 1H), 5.75 - 5.70 (m, 1H), 4.16 (t, J = 5.6 Hz, 2H), 3.71 (t, J = 5.5 Hz, 2H), 3.24 (s, 3H), 2.29 (s, 3H), 2.11 (s, 3H) |
| Example 203 N-[1-[5-fluoro-2-[[1-(2- isopropoxyethyl)pyrazol-4- yl]amino]pyrimidin-4-yl]-3-methyl- indol-5-yl]prop-2-enamide | 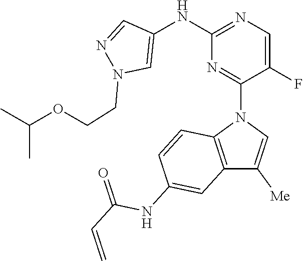 [SMILES: C=CC(=O)Nc1ccc2c(c1)c(C)cn2-c1nc(Nc2cnn(CCOC(C)C)c2)ncc1F] | LCMS m/z = 464 (M + 1); 1H NMR (400 MHz, DMSO-d6) δ: 9.89 (s, 1H), 9.22 (s, 1H), 8.47 (d, J = 4.4 Hz, 1H), 8.32 - 8.24 (m, 1H), 7.98 (s, 1H), 7.86 (s, 1H), 7.62 (s, 1H), 7.55 (s, 1H), 7.52 - 7.47 (m, 1H), 6.52 - 6.42 (m, 1H), 6.30 - 6.23 (m, 1H), 5.74 - 5.69 (m, 1H), 4.17 (t, J = 5.5 Hz, 2H), 3.72 (t, J = 5.7 Hz, 2H), 3.57 - 3.49 (m, 1H), 2.30 (s, 3H), 1.05 (d, J = 6.3 Hz, 6H). |
| Example 204 N-[1-[5-fluoro-2-[[1-(2- methoxypropyl)pyrazol-4- yl]amino]pyrimidin-4-yl]-3-methyl- indol-5-yl]prop-2-enamide | 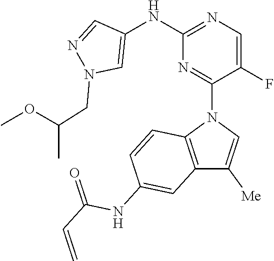 [SMILES: C=CC(=O)Nc1ccc2c(c1)c(C)cn2-c1nc(Nc2cnn(CC(C)OC)c2)ncc1F] | LCMS m/z = 450 (M + 1); 1H NMR (DMSO-d6, 400 MHz) δ: 9.91 (br s, 1H), 9.25 (br s, 1H), 8.48 (d, J = 4.5 Hz, 1H), 8.28 (br d, J = 8.8 Hz, 1H), 7.99 (d, J = 2.0 Hz, 1H), 7.83 (s, 1H), 7.62 (s, 1H), 7.55 (s, 1H), 7.49 (br d, J = 8.7 Hz, 1H), 6.47 (dd, J = 17.0, 10.0 Hz, 1H), 6.20-6.32 (m, 1H), 5.72 (dd, J = 10.4, 1.3 Hz, 1H), 4.00-4.13 (m, 2H), 3.64- 3.75 (m, 1H), 3.21 (s, 3H), 2.29 (s, 3H), 1.07 (d, J = 6.4 Hz, 3H) |
| Example 205 N-[1-[5-fluoro-2-[1-[(2S)-2- methoxypropyl]pyrazol-4- yl]amino]pyrimidin-4-yl]-3-methyl- indol-5-yl]prop-2-enamide | 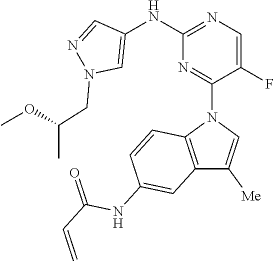 [SMILES: C=CC(=O)Nc1ccc2c(c1)c(C)cn2-c1nc(Nc2cnn(C[C@H](C)OC)c2)ncc1F] | LCMS m/z = 450 (M + 1); 1H NMR (500 MHz, DMSO-d6) δ: 9.93 (s, 1H), 9.27 (s, 1H), 8.50 (d, J = 4.6 Hz, 1H), 8.30 (br d, J = 8.5 Hz, 1H), 8.00 (s, 1H), 7.84 (s, 1H), 7.63 (s, 1H), 7.56 (s, 1H), 7.50 (br d, J = 7.9 Hz, 1H), 6.48 (dd, J = 16.9, 10.2 Hz, 1H), 6.28 (d, J = 17.1 Hz, 1H), 5.73 (d, J = 10.1 Hz, 1H), 4.03-4.12 (m, 2H), 3.67- 3.74 (m, 1H), 3.22 (s, 3H), 2.30 (s, 3H), 1.08 (d, J = 6.1 Hz, 3H) |
| Example 206 N-[1-[5-fluoro-2-[[1-[(1- hydroxycyclopropyl)methyl]-3- methyl-pyrazol-4-yl]amino]pyrimidin- 4-yl]-3-methyl-indol-5-yl]prop-2- enamide N-[1-[5-fluoro-2-[[1-[(1- hydroxycyclopropyl)methyl]-5- methyl-pyrazol-4-yl]amino]pyrimidin- 4-yl]-3-methyl-indol-5-yl]prop-2- enamide | 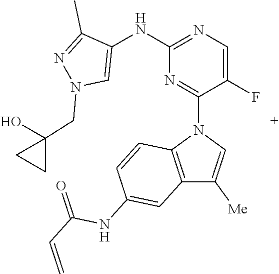 [SMILES: C=CC(=O)Nc1ccc2c(c1)c(C)cn2-c1nc(Nc2cn(CC3(O)CC3)nc2C)ncc1F] | LCMS m/z = 462 (M + 1) |
| | 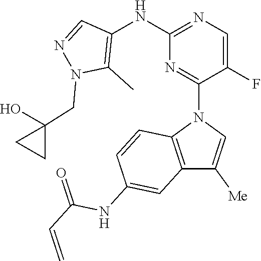 [SMILES: C=CC(=O)Nc1ccc2c(c1)c(C)cn2-c1nc(Nc2cnn(CC3(O)CC3)c2C)ncc1F] | |
| Example 207 N-[1-[2-(5,6-dihydro-4H-pyrrolo[1,2- b]pyrazol-3-ylamino)-5-fluoro- pyrimidin-4-yl]-3-methyl-indol-5- yl]prop-2-enamide | 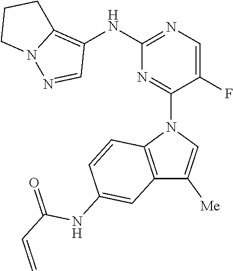 [SMILES: C=CC(=O)Nc1ccc2c(c1)c(C)cn2-c1nc(Nc2cnn3c2CCC3)ncc1F] | LCMS m/z = 418 (M + 1); 1H NMR (500 MHz, DMSO-d6) δ: 9.92 (br s, 1H), 8.80 (s, 1H), 8.43(d, J = 4.6 Hz, 1H), 8.19 (br d, J = 8.5 Hz, 1H), 7.99 (s, 1H), 7.64 (s, 1H), 7.51 (s, 1H), 7.42 (br d, J = 8.9 Hz, 1H), 6.47 (dd, J = 17.1, 10.4 Hz, 1H), 6.30 - 6.25 (m, 1H), 5.73 (d, J = 10.2 Hz, 1H), 4.07 (t, J = 7.3 Hz, 2H), 2.83 (br t, J = 7.3 Hz, 2H), 2.48 - 2.44 (m, 2H), 2.29 (s, 3H) |
| Example 208 N-[1-[5-fluoro-2-[(1-tetrahydropyran- 4-ylpyrazol-4-yl)amino]pyrimidin-4- yl]-3-methyl-indol-5-yl]prop-2- enamide | 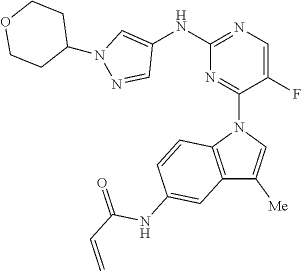 [SMILES: C=CC(=O)Nc1ccc2c(c1)c(C)cn2-c1nc(Nc2cnn(C3CCOCC3)c2)ncc1F] | LCMS m/z = 462.5 (M + 1); 1H NMR (500 MHz, DMSO-d6) δ: 9.89 (s, 1H), 9.21 (s, 1H), 8.48 (d, J = 4.3 Hz, 1H), 8.27 (d, J = 8.6 Hz, 1H), 7.97 (s, 1H), 7.90 (s, 1H), 7.62 (s, 1H), 7.56 (s, 1H), 7.51 (d, J = 8.9 Hz, 1H), 6.47 (dd, J = 17.2, 10.0 Hz, 1H), 6.27 (d, J = 16.9 Hz, 1H), 5.72 (d, J = 10.3 Hz, 1H), 4.40-4.27 (m, 1H), 4.05-3.86 (m, 2H), 3.58-3.38 (m, 2H), 2.30 (s, 3H), 2.06- 1.86 (m, 4H) |
| Example 209 N-[1-[6-[(1,3-dimethylpyrazol-4- yl)amino]-3-methyl-pyridazin-4-yl]-3- methyl-indol-5-yl]prop-2-enamide | 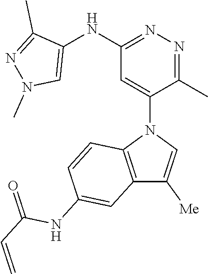 [SMILES: C=CC(=O)Nc1ccc2c(c1)c(C)cn2-c1cc(Nc2cn(C)nc2C)nnc1C] | LCMS m/z = 403 (M + 1); 1H NMR (400 MHz, DMSO-d6) δ: 10.12 (s, 1H), 8.47 (s, 1H), 8.04 (d, J = 2.0 Hz, 1H), 8.02 (s, 1H), 7.37-7.43 (m, 2H), 7.23 (d, J = 9.0 Hz, 1H), 7.00 (s, 1H), 6.43 (dd, J = 17.0, 10.1 Hz, 1H), 6.26 (dd, J = 17.0, 2.1 Hz, 1H), 5.73 (dd, J = 10.0, 2.1 Hz, 1H), 3.74 (s, 3H), 2.35 (s, 3H), 2.28 (d, J = 1.1 Hz, 3H), 2.14 (s, 3H) |
| Example 210 N-[1-[5-fluoro-2-[[1-[(2R)-2- methoxypropyl]pyrazol-4- yl]amino]pyrimidin-4-yl]-3-methyl- indol-5-yl]prop-2-enamide | 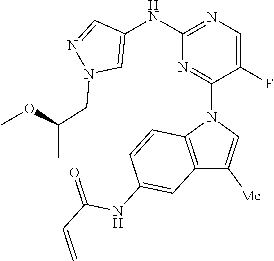 [SMILES: C=CC(=O)Nc1ccc2c(c1)c(C)cn2-c1nc(Nc2cnn(C[C@@H](C)OC)c2)ncc1F] | LCMS m/z = 450 (M + 1); 1H NMR (500 MHz, DMSO-d6) δ: 9.94 (s, 1H), 9.28 (s, 1H), 8.50 (d, J = 4.6 Hz, 1H), 8.30 (d, J = 9.2 Hz, 1H), 8.00 (s, 1H), 7.84 (s, 1H), 7.63 (s, 1H), 7.56 (s, 1H), 7.50 (d, J = 8.9 Hz, 1H), 6.48 (dd, J = 16.9, 10.2 Hz, 1H), 6.28 (dd, J = 16.9, 1.7 Hz, 1H), 5.73 (dd, J = 10.2, 1.7 Hz, 1H), 4.04-4.12 (m, 2H), 3.67-3.74 (m, 1H), 3.22 (s, 3H), 2.30 (s, 3H), 1.08 (d, J = 6.4 Hz, 3H) |
| Example 211 N-[1-[5-fluoro-2-[[3-methyl-1- (oxetan-3-yl)pyrazol-4- yl]amino]pyrimidin-4-yl]-3-methyl- indol-5-yl]prop-2-enamide N-[1-[5-fluoro-2-[[5-methyl-1- (oxetan-3-yl)pyrazol-4- yl]amino]pyrimidin-4-yl]-3-methyl- indol-5-yl]prop-2-enamide | 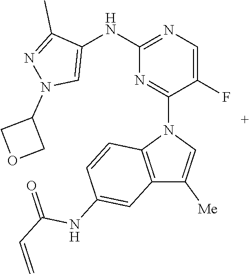 [SMILES: C=CC(=O)Nc1ccc2c(c1)c(C)cn2-c1nc(Nc2cn(C3COC3)nc2C)ncc1F] | LCMS m/z = 448 (M + 1) |
| | 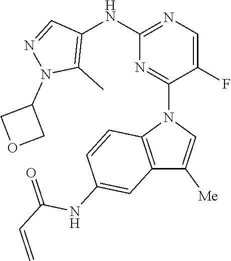 [SMILES: C=CC(=O)Nc1ccc2c(c1)c(C)cn2-c1nc(Nc2cnn(C3COC3)c2C)ncc1F] | |
| Example 212 N-[1-[5-fluoro-2-[[1-(2- isopropoxyethyl)-3-methyl-pyrazol-4- yl]amino]pyrimidin-4-yl]-3-methyl- indol-5-yl]prop-2-enamide N-[1-[5-fluoro-2-[[1-(2- isopropoxyethyl)-5-methyl-pyrazol-4- yl]amino]pyrimidin-4-yl]-3-methyl- indol-5-yl]prop-2-enamide | 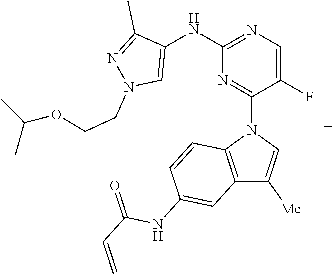 [SMILES: C=CC(=O)Nc1ccc2c(c1)c(C)cn2-c1nc(Nc2cn(CCOC(C)C)nc2C)ncc1F] | LCMS m/z = 478 (M + 1) |
| | 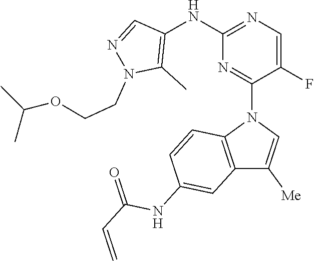 [SMILES: C=CC(=O)Nc1ccc2c(c1)c(C)cn2-c1nc(Nc2cnn(CCOC(C)C)c2C)ncc1F] | |
| Example 213 (E)-N-[1-[2-[(1,3-dimethylpyrazol-4- yl)amino]-5-fluoro-pyrimidin-4-yl]-3- methyl-pyrrolo[2,3-b]pyridin-5-yl]-4- morpholino-but-2-enamide | 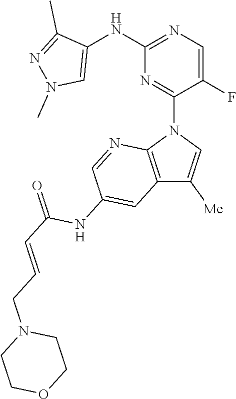 [SMILES: Cc1nn(C)cc1Nc1ncc(F)c(-n2cc(C)c3cc(NC(=O)/C=C/CN4CCOCC4)cnc32)n1] | LCMS m/z = 506 (M + 1); 1H NMR (500 MHz, DMSO-d6) δ: 10.03 (bs, 1H), 8.71 (bs, 1H), 8.54 (d, J = 3.1 Hz, 1H), 8.51 - 8.47 (m, 1H), 8.39 (d, J = 2.1 Hz, 1H), 7.97 (s, 1H), 7.60 (s, 1H), 6.80 (td, J = 6.0, 15.5 Hz, 1H), 6.32 (d, J = 15.6 Hz, 1H), 3.74 (bs, 3H), 3.63 (t, J = 4.3 Hz, 4H), 3.17 (d, J = 5.8 Hz, 2H), 2.45 (t, J = 4.5 Hz, 4H), 2.32 (s, 3H), 2.14 (s, 3H) |
| Example 214 N-[1-[5-fluoro-2-[[1-[(2R)-2- hydroxypropyl]-3-methyl-pyrazol-4- yl]amino]pyrimidin-4-yl]-3-methyl- indol-5-yl]prop-2-enamide | 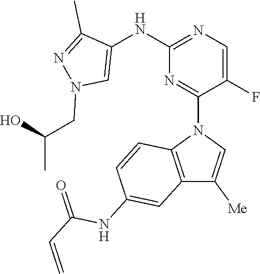 [SMILES: C=CC(=O)Nc1ccc2c(c1)c(C)cn2-c1nc(Nc2cn(C[C@@H](C)O)nc2C)ncc1F] | LCMS m/z = 450 (M + 1); 1H NMR(500 MHz, DMSO-d6) δ: 9.88 (bs, 1H), 8.49 (s, 1H), 8.42 (d, J = 4.7 Hz, 1H), 8.17 (d, J = 8.8 Hz, 1H), 7.98 (s, 1H), 7.74 (s, 1H), 7.63 (s, 1H), 7.41 (d,J = 9.0 Hz, 1H), 6.47 (dd, J = 16.8, 10.2 Hz, 1H), 6.27 (d, J = 17.2 Hz, 1H), 5.72 (d, J = 10.4 Hz, 1H), 4.54 (d, J = 4.7 Hz, 1H), 4.08 - 3.86 (m, 3H), 2.28 (s, 3H), 2.11 (s, 3H), 1.08 (d, J = 6.1 Hz, 3H) |
| Example 215 N-[1-[5-fluoro-2-[(1-tetrahydrofuran- 3-ylpyrazol-4-yl)amino]pyrimidin-4- yl]-3-methyl-indol-5-yl]prop-2- enamide | 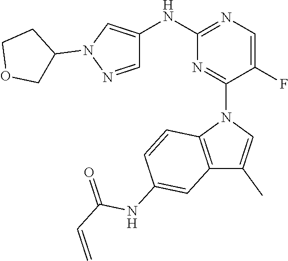 [SMILES: C=CC(=O)Nc1ccc2c(c1)c(C)cn2-c1nc(Nc2cnn(C3CCOC3)c2)ncc1F] | LCMS m/z = 448 (M + 1); 1H NMR: (500 MHz, DMSO-d6, 80° C.) δ: 9.90 (bs, 1H), 9.24 (s, 1H), 8.49 (d, J = 4.7 Hz, 1H), 8.27 (d, J = 8.6 Hz, 1H), 8.00 (s, 1H), 7.91 (s, 1H), 7.63 (s, 1H), 7.57 (s, 1H), 7.50 (d,J = 8.6 Hz, 1H), 6.48 (dd, J = 17.0, 10.2 Hz, 1H), 6.28 (dd, J = 16.8, 1.2 Hz, 1H), 5.72 (dd, J = 10.3, 1.3 Hz, 1H), 5.00-4.92 (m, 1H), 4.03- 3.88 (m, 3H), 3.85-3.78 (m, 1H), 2.43- 2.34 (m, 1H), 2.32-2.23 (m, 1H), 2.30 (s, 3H) |
| Example 216 N-[1-[5-fluoro-2-[[1-[(2R)-2- hydroxypropyl]-5-methyl-pyrazol-4- yl]amino]pyrimidin-4-yl]-3-methyl- indol-5-yl]prop-2-enamide | 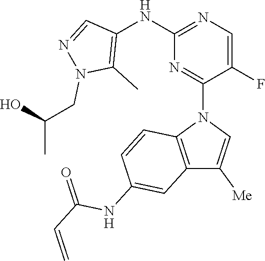 [SMILES: C=CC(=O)Nc1ccc2c(c1)c(C)cn2-c1nc(Nc2cnn(C[C@@H](C)O)c2C)ncc1F] | LCMS m/z = 450 (M + 1); 1H NMR (600 MHz, DMSO-d6) δ: 9.86 (br s, 1H), 8.44 (br s, 1H), 8.38 (d, J = 4.8 Hz, 1H), 8.12 (br d, J = 8.4 Hz, 1H), 7.96 (d, J = 2.0 Hz, 1H), 7.61 (dd, J = 2.0, 1.3 Hz, 1H), 7.47 (s, 1H), 7.37 (dd, J = 9.0, 2.0 Hz, 1H), 6.45 (dd, J = 17.0, 10.2 Hz, 1H), 6.26 (dd, J = 17.1, 1.8 Hz, 1H), 5.70 (dd, J = 10.2, 1.9 Hz, 1H), 4.56 (br s, 1H), 4.01-4.08 (m, 1H), 3.97-4.01 (m, 1H), 3.89-3.94 (m, 1H), 2.26 (d, J = 1.3 Hz, 3H), 2.17 (s, 3H), 1.09 (d, J = 6.1 Hz, 3H) |
| Example 217 N-[1-[6-[(1,3-dimethylpyrazol-4- yl)amino]-3-methyl-pyridazin-4-yl]-3- methyl-indol-5-yl]prop-2-enamide | 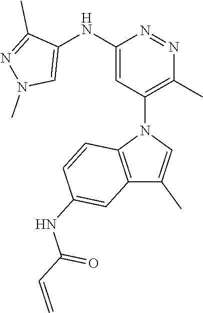 [SMILES: C=CC(=O)Nc1ccc2c(c1)c(C)cn2-c1cc(Nc2cn(C)nc2C)nnc1C] | LCMS m/z = 402 (M + 1); 1H NMR (400 MHz, DMSO-d6) δ: 10.12 (s, 1H), 8.47 (s, 1H), 8.04 (d, J = 2.0 Hz, 1H), 8.02 (s, 1H), 7.37-7.43 (m, 2H), 7.23 (d, J = 9.0 Hz, 1H), 7.00 (s, 1H), 6.43 (dd, J = 17.0, 10.1 Hz, 1H), 6.26 (dd, J = 17.0, 2.1 Hz, 1H), 5.73 (dd, J = 10.0, 2.1 Hz, 1H), 3.74 (s, 3H), 2.35 (s, 3H), 2.28 (d, J = 1.1 Hz, 3H), 2.14 (s, 3H) |
| Example 218 N-[3-methyl-1-[4-[(1-methylpyrazol- 4-yl)amino]-1,3,5-triazin-2-yl]indol-5- yl]prop-2-enamide | 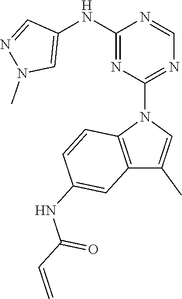 [SMILES: C=CC(=O)Nc1ccc2c(c1)c(C)cn2-c1ncnc(Nc2cnn(C)c2)n1] | LCMS m/z = 375 (M + 1); 1H NMR (500 MHz, DMSO-d6) δ: 9.94 (br s, 1H), 9.91 (br s, 1H), 8.61 (br s, 2H), 7.99 (s, 1H), 7.96 (s, 1H), 7.90 (s, 1H), 7.60 (s, 1H), 7.52 (br s, 1H), 6.48 (dd, J = 16.9, 10.2 Hz, 1H), 6.28 (d, J = 17.1 Hz, 1H), 5.73 (d, J = 11.0 Hz, 1H), 3.87 (br s, 3H), 2.29 (s, 3H) |
| Example 219 N-[3-methyl-1-[6-[(1-methylpyrazol- 4-yl)amino]pyrimidin-4-yl]indol-5- yl]prop-2-enamide | 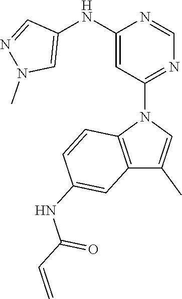 [SMILES: C=CC(=O)Nc1ccc2c(c1)c(C)cn2-c1cc(Nc2cnn(C)c2)ncn1] | LCMS m/z = 374 (M + 1); 1H NMR: (500 MHz, DMSO-d6) δ: 9.86 (br s, 1H), 9.12 (br s, 1H), 8.52 (s, 1H), 8.28 (d, J = 8.9 Hz, 1H), 7.98 (s, 1H), 7.91 (s, 1H), 7.70 (s, 1H), 7.52 (s, 1H), 7.48 (br d, J = 9.5 Hz, 1H), 6.73 (s, 1H), 6.48 (dd, J = 16.9, 10.2 Hz, 1H), 6.27 (d, J = 17.1 Hz, 1H), 5.72 (d, J = 10.7 Hz, 1H), 3.85 (s, 3H), 2.30 (s, 3H) |
| Example 220 N-[1-[4-[(1,3-dimethylpyrazol-4- yl)amino]-1,3,5-triazin-2-yl]-3- methyl-indol-5-yl]prop-2-enamide | 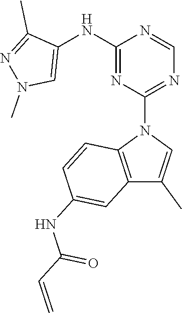 [SMILES: C=CC(=O)Nc1ccc2c(c1)c(C)cn2-c1ncnc(Nc2cn(C)nc2C)n1] | LCMS m/z = 388 (M + 1); 1H NMR: (500 MHz, DMSO-d6) δ: 9.88 (bs, 1H), 9.26 (s, 1H), 8.1-8.8 (b, 1H), 8.54 (s, 1H), 7.97 (d, J = 1.2 Hz, 1H), 7.92 (bs, 1H), 7.78 (s, 1H), 7.45 (bs, 1H), 6.46 (dd, J = 16.7, 10.1 Hz, 1H), 6.27 (dd, J = 16.7, 1.5 Hz, 1H), 5.72 (dd, J = 10.0, 1.7 Hz, 1H), 3.81 (s, 3H), 2.27 (s, 3H), 2.13 (s, 3H) |
| Example 221 N-[3-methyl-1-[3-methyl-6-[(1- methylpyrazol-4-yl)amino]pyridazin- 4-yl]indol-5-yl]prop-2-enamide | 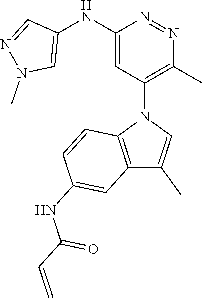 [SMILES: C=CC(=O)Nc1ccc2c(c1)c(C)cn2-c1cc(Nc2cnn(C)c2)nnc1C] | LCMS m/z = 388 (M + 1); 1H NMR (400 MHz, DMSO-d6) δ: 10.13 (s, 1H), 9.11 (s, 1H), 8.04- 8.07 (m, 2H), 7.48 (d, J = 0.6 Hz, 1H), 7.42 (d, J = 1.0 Hz, 1H), 7.36-7.40 (m, 1H), 7.24 (d, J = 8.8 Hz, 1H), 6.93 (s, 1H), 6.45 (dd, J = 16.9, 10.0 Hz, 1H), 6.24 (dd, J = 17.0, 2.1 Hz, 1H), 5.70-5.75 (dd, J = 10.0, 2.1 Hz, 1H), 3.82 (s, 3H), 2.35 (s, 3H), 2.28 (d, J = 1.0 Hz, 3H) |
| Example 222 N-[1-[6-[(1,3-dimethylpyrazol-4- yl)amino]pyrimidin-4-yl]-3-methyl- indol-5-yl]prop-2-enamide | 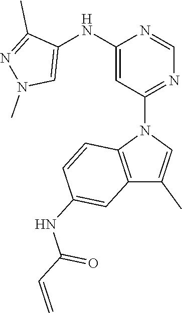 [SMILES: C=CC(=O)Nc1ccc2c(c1)c(C)cn2-c1cc(Nc2cn(C)nc2C)ncn1] | LCMS m/z = 388 (M + 1); 1H NMR: (500 MHz, DMSO-d6) δ: 9.86 (bs, 1H), 8.54 (s, 1H), 8.47 (s, 1H), 8.26 (d, J = 8.9 Hz, 1H), 7.97 (s, 1H), 7.84 (s, 1H), 7.66 (s, 1H), 7.47 (d, J = 8.9 Hz, 1H), 6.69 (s, 1H), 6.47 (dd, J = 16.7, 9.8 Hz, 1H), 6.26 (d, J = 17.1 Hz, 1H), 5.71 (d, J = 10.3 Hz, 1H), 3.77 (s, 3H), 2.28 (s, 3H), 2.13 (s, 3H) |
| Example 223 N-[3-methyl-1-[5-methyl-2-[(1- methylpyrazol-4-yl)amino]-4- pyridyl]indol-5-yl]prop-2-enamide | 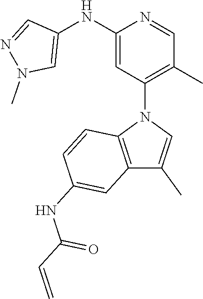 [SMILES: C=CC(=O)Nc1ccc2c(c1)c(C)cn2-c1cc(Nc2cnn(C)c2)ncc1C] | LCMS m/z = 387 (M + 1); 1H NMR (DMSO-d6, 500 MHz) δ: 10.09 (s, 1H), 8.78 (s, 1H), 8.14 (s, 1H), 8.04 (s, 1H), 7.93 (s, 1H), 7.40 (s, 1H), 7.36 (d, J = 9.2 Hz, 1H), 7.32 (s, 1H), 7.15 (d, J = 8.5 Hz, 1H), 6.60 (s, 1H), 6.46 (dd, J = 16.9, 10.2 Hz, 1H), 6.25 (dd, J = 16.8, 1.8 Hz, 1H), 5.73 (dd, J = 10.1, 1.8 Hz, 1H), 3.80 (s, 3H), 2.29 (s, 3H), 1.99 (s, 3H) |
| Example 224 N-[1-[2-[(1,3-dimethylpyrazol-4- yl)amino]-5-methyl-4-pyridyl]-3- methyl-indol-5-yl]prop-2-enamide | 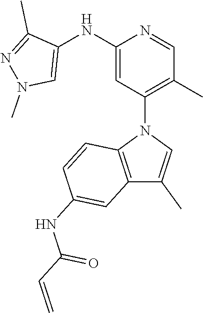 [SMILES: C=CC(=O)Nc1ccc2c(c1)c(C)cn2-c1cc(Nc2cn(C)nc2C)ncc1C] | LCMS m/z = 401 (M + 1); 1H NMR (DMSO-d6, 500 MHz) δ: 10.09 (s, 1H), 8.08-8.13 (m, 2H), 8.03 (s, 1H), 7.87 (s, 1H), 7.37 (d, J = 8.5 Hz, 1H), 7.30 (s, 1H), 7.13 (d, J = 8.9 Hz, 1H), 6.61 (s, 1H), 6.46 (dd, J = 16.9, 10.2 Hz, 1H), 6.25 (dd, J = 17.1, 1.8 Hz, 1H), 5.73 (dd, J = 10.1, 1.8 Hz, 1H), 3.72 (s, 3H), 2.28 (s, 3H), 2.09 (s, 3H), 1.99 (s, 3H) |
| Example 225 N-[1-[6-chloro-3-[(1,3- dimethylpyrazol-4-yl)amino]-1,2,4- triazin-5-yl]-3-methyl-indol-5- yl]prop-2-enamide | 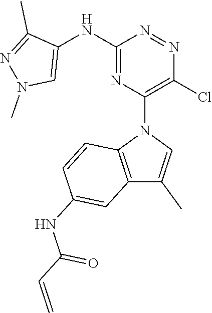 [SMILES: C=CC(=O)Nc1ccc2c(c1)c(C)cn2-c1nc(Nc2cn(C)nc2C)nnc1Cl] | LCMS m/z = 423 (M + 1); 1H NMR (DMSO-d6, 500 MHz) δ: 9.93 (br s, 1H), 9.15-9.31 (m, 1H), 7.99 (d, J = 1.5 Hz, 1H), 7.95 (br d, J = 8.9 Hz, 1H), 7.89 (s, 1H), 7.76 (s, 1H), 7.45 (dd, J = 8.9, 1.2 Hz, 1H), 6.47 (dd, J = 16.9, 10.2 Hz, 1H), 6.28 (dd, J = 17.1, 1.8 Hz, 1H), 5.73 (dd, J = 10.1, 1.8 Hz, 1H), 3.77 (s, 3H), 2.29 (s, 3H), 2.13 (s, 3H) |
| Example 226 N-[1-[5-fluoro-2-[(1-methylpyrazol-4- yl)amino]pyrimidin-4-yl]-2,3- dimethyl-indol-5-yl]prop-2-enamide | 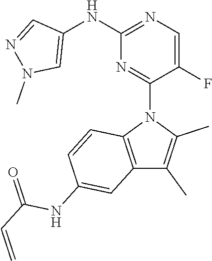 [SMILES: C=CC(=O)Nc1ccc2c(c1)c(C)c(C)n2-c1nc(Nc2cnn(C)c2)ncc1F] | LCMS m/z = 406 (M + 1); 1H NMR (600 MHz, DMSO-d6) δ 10.12 (s, 1H), 9.81 - 9.69 (m, 1H), 8.71 (d, J = 2.2 Hz, 1H), 7.97 (s, 1H), 7.80 (br s, 1H), 7.48 (s, 1H), 7.33 (dd, J = 8.9, 1.6 Hz, 1H), 7.26 (dd, J = 8.9, 2.8 Hz, 1H), 6.46 (dd, J = 16.9, 10.1 Hz, 1H), 6.25 (dd, J = 16.9, 1.7 Hz, 1H), 5.73 (dd, J = 10.1, 1.7 Hz, 1H), 3.77 (br s, 3H), 2.36 (s, 3H), 2.22 (s, 3H) |
| Example 227 N-[1-[5-fluoro-2-[(1-methylpyrazol-4- yl)amino]pyrimidin-4-yl]-2-methyl- indol-5-yl]prop-2-enamide | 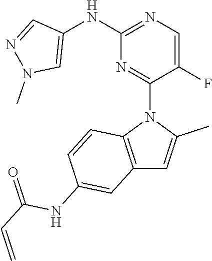 [SMILES: C=CC(=O)Nc1ccc2c(c1)cc(C)n2-c1nc(Nc2cnn(C)c2)ncc1F] | LCMS m/z = 392 (M + 1); 1H NMR (500 MHz, DMSO-d6) δ 9.80 (s, 1H), 9.46 (s, 1H), 8.68 (d, J = 2.4 Hz, 1H), 7.93 (s, 1H), 7.76 (s, 1H), 7.49 (s, 1H), 7.33 (br d, J = 7.9 Hz, 1H), 7.24 (dd, J = 8.9, 2.4 Hz, 1H), 6.50 (s, 1H), 6.46 (dd, J = 16.9, 10.2 Hz, 1H), 6.25 (dd, J = 17.0, 2.0 Hz, 1H), 5.70 (dd, J = 10.2, 2.0 Hz, 1H), 3.78 (s, 3H), 2.43 (s, 3H) |
| Example 228 N-[9-[5-fluoro-2-[(1-methylpyrazol-4- yl)amino]pyrimidin-4-yl]carbazol-3- yl]prop-2-enamide | 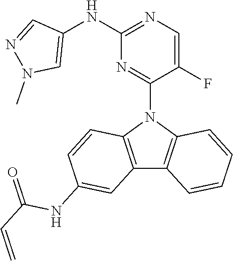 [SMILES: C=CC(=O)Nc1ccc2c(c1)c1ccccc1n2-c1nc(Nc2cnn(C)c2)ncc1F] | LCMS m/z = 428 (M + 1); 1H NMR (500 MHz, DMSO-d6) δ 10.33 (s, 1H), 9.83 (br s, 1H), 8.78 (d, J = 2.7 Hz, 1H), 8.62 (s, 1H), 8.16 (d, J = 7.6 Hz, 1H), 7.81 (br s, 1H), 7.65-7.71 (m, 3H), 7.49-7.53 (m, 2H), 7.36-7.40 (m, 1H), 6.50 (dd, J = 17.1, 10.1 Hz, 1H), 6.31 (dd, J = 16.8, 1.8 Hz, 1H), 5.78 (dd, J = 10.2, 1.7 Hz, 1H), 3.75 (br s, 3H) |
| Example 229 N-[4-[5-fluoro-2-[(1-methylpyrazol-4- yl)amino]pyrimidin-4-yl]-2,3- dihydro-1H-cyclopenta[b]indol-7- yl]prop-2-enamide | 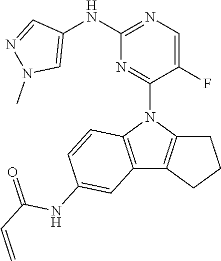 [SMILES: C=CC(=O)Nc1ccc2c(c1)c1c(n2-c2nc(Nc3cnn(C)c3)ncc2F)CCC1] | LCMS m/z = 418 (M + 1); 1H NMR (500 MHz, DMSO-d6) δ 9.84 (s, 1H), 9.33 (s, 1H), 8.58 (d, J = 3.1 Hz, 1H), 7.87 (s, 1H), 7.78 (s, 1H), 7.57 (dd, J = 8.9, 2.4 Hz, 1H), 7.50 (s, 1H), 7.36 (d, J = 8.1 Hz, 1H), 6.46 (dd, J = 16.9, 10.2 Hz, 1H), 6.26 (dd, J = 16.9, 1.7 Hz, 1H), 5.71 (dd, J = 10.2, 1.7 Hz, 1H), 3.78 (s, 3H), 2.99 (br t, J = 6.4 Hz, 2H), 2.81 (t, J = 6.7 Hz, 2H), 2.52 (br s, 2H) |
| Example 230 N-[9-[5-fluoro-2-[(1-methylpyrazol-4- yl)amino]pyrimidin-4-yl]-5,6,7,8- tetrahydrocarbazol-3-yl]prop-2- enamide | 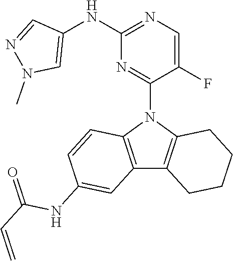 [SMILES: C=CC(=O)Nc1ccc2c(c1)c1c(n2-c2nc(Nc3cnn(C)c3)ncc2F)CCCC1] | LCMS m/z = 432 (M + 1); 1H NMR (500 MHz, DMSO-d6) δ: 9.83 (s, 1H), 9.38 (s, 1H), 8.62 (d, J = 2.4 Hz, 1H), 7.87 (s, 1H), 7.77 (s, 1H), 7.49 (s, 1H), 7.38 (d, J = 9.2 Hz, 1H), 7.30 (dd, J = 8.7, 2.9 Hz, 1H), 6.46 (dd, J = 16.9, 10.2 Hz, 1H), 6.25 (d, J = 16.8 Hz, 1H), 5.70 (d, J = 11.9 Hz, 1H), 3.78 (s, 3H), 2.78 (br s, 2H), 2.69 (br s, 2H), 1.87 (br s, 4H) |

Kinase Assays

Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. Streptavidin-coated magnetic beads were treated with biotinylated small molecule ligands for 30 minutes at room temperature to generate affinity resins for kinase assays. The liganded beads were blocked with excess biotin and washed with blocking buffer SeaBlock (Pierce), 1% BSA, 0.05% Tween 20, 1 mM DTT to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT).

Test compounds were prepared as 111× stocks in 100% DMSO. Kds were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR.

Binding Constants (Kds)

Binding constants were calculated with a standard dose-response curve using the Hill equation:

$$\\ Response = Background + \frac{Signal - Background}{1 + ( \frac{{Kd}^{{}^{} Hill Slope}}{{Dose}^{{}^{} Hill Slope}} )}$$

The Hill Slope was set to −1. Curves were fitted using a non-linear least square fit with the Levenberg-Marquardt algorithm.

Cell lines used for Cell Viability Assays

| Cell line | Mutation or Fusion | Source |
|---|---|---|
| RT112/84 | FGFR3-TACC3 fusion | American Type Culture |
| (Bladder Cancer) | | Collection (Manassas, VA) |
| UM-UC-14 | FGFR3(S249C) | Sigma, (St. Louis, MO) |
| (Bladder Cancer) | | |
| SNU-16 | FGFR2 amplification | American Type Culture |
| (Gastric Carcinoma) | | Collection (Manassas, VA) |
| KG-1 (Acute | FGFR1OP2-FGFR1 | American Type Culture |
| Myeloid Leukemia) | fusion | Collection (Manassas, VA) |

RT112/84, UM-UC-14, SNU-16 and KG-1 Cancer Cell Line Cell Viability Assays

To detect the change of intracellular ATP by Cell Titer-Glo@ and to evaluate the inhibitory effect of the compounds on cancer cell lines by determining the in vitro IC₅₀ value of the compounds.

Cell Titer-Glo@ 2.0 Luminescent cell viability assay reagent was purchased from Promega (Madison, WI). RT112/84, SNU-16, and KG-1 cell lines were purchased from American Type Culture Collection (Manassas, VA). UM-UC-14 cell line was purchased from Sigma (St. Louis, MO). RT112/84, UM-UC-14, SNU-16, and KG-1 cells were cultured in RPMI1640 media supplemented with 10% fetal bovine serum. Cultures were maintained at 37° C. in a humidified atmosphere of 5% CO2 and 95% air.

Cell Viability Assay Procedure: Cells were plated in 96-well clear bottom/white plates (Corning #3903) at 10,000 cells/well in 100 μl of media, incubated overnight. The next day, test compound DMSO stock solutions were made at 10 mM and 2 μM final concentration. Compounds were then added to cells in a 9-dose, 10-fold dilution series starting at 30 μM with an HP 300e Digital Dispenser (each dose was applied in triplicate). DMSO was backfilled to each well up to 301 nL total volume of test compound+DMSO, and a total of 301 nL DMSO was added to a control/no test compound well in triplicate. The cells in cell culture plates were incubated with the compounds at 37° C. and 5% CO₂ for 72 hours. Then 50 μl of Cell Titer Glo 2.0 reagent was added to each well of the cell culture plates. The contents were covered from light and mixed on an orbital shaker at room temperature for 10 min. Luminescence was recorded by a Synergy H1 Microplate Reader (Biotek, Winooski, VT). Cells were assessed as a percentage of DMSO only treated control cells. Curves were plotted and IC₅₀ values were calculated using the GraphPad Prism 8 program based on a sigmoidal dose-response equation (4 parameter). The Kd values are shown in Table 2

TABLE 2
FGFR binding data

| | | | | FGFR3 |
|---|---|---|---|---|
| | FGFR1 | FGFR2 | FGFR3 | (V555M) |
| Example | (Kd nM) | (Kd nM) | (Kd nM) | (Kd nM) |
| 1. | A | A | A | A |
| 2. | A | A | A | A |
| 3. | A | A | A | A |
| 4. | A | C | B | A |
| 5. | C | B | B | A |
| 6. | C | C | C | A |
| 7. | A | B | B | A |
| 8. | A | A | A | A |
| 9. | A | A | A | A |
| 10. | B | B | A | A |
| 11. | D | D | D | D |
| 12. | C | D | B | A |
| 13. | D | D | D | D |
| 14. | C | D | D | B |
| 15. | B | A | A | A |
| 16. | C | C | A | A |
| 17. | C | C | B | A |
| 18. | B | B | B | A |
| 19. | A | A | A | A |
| 20. | D | D | D | D |
| 21. | C | A | B | A |
| 22. | A | A | A | A |
| 23. | A | A | A | A |
| 24. | A | A | A | A |
| 25. | A | A | A | A |
| 26. | A | A | A | A |
| 27. | A | A | A | A |
| 28. | A | A | A | A |
| 29. | D | D | D | D |
| 30. | A | A | A | A |
| 31. | A | A | A | A |
| 32. | A | A | A | A |
| 33. | D | D | D | D |
| 34. | A | A | A | A |
| 35. | A | A | A | A |
| 36. | A | A | A | A |
| 37. | B | A | B | A |
| 38. | A | A | A | A |
| 39. | D | D | D | D |
| 40. | A | A | A | A |
| 41. | B | A | A | A |
| 42. | A | A | A | A |
| 44. | A | A | A | A |
| 45. | A | A | A | A |
| 46. | A | A | A | A |
| 47. | B | A | A | A |
| 48. | A | A | A | A |
| 49. | B | A | A | A |
| 51. | A | A | A | A |
| 52. | A | A | A | A |
| 53. | A | A | A | A |
| 54. | A | A | A | A |
| 55. | A | A | A | A |
| 56. | B | A | A | A |
| 57. | A | A | A | A |
| 58. | A | A | A | A |
| 59. | A | A | A | A |
| 60. | D | D | D | B |
| 61. | A | A | A | A |
| 62. | C | A | A | A |
| 63. | C | A | A | A |
| 64. | A | A | A | A |
| 65. | A | A | A | A |
| 66. | A | A | A | A |
| 67. | A | A | A | A |
| 68. | A | A | A | A |
| 69. | B | A | A | A |
| 70. | B | A | A | A |
| 71. | D | D | D | D |
| 72. | A | A | A | A |
| 73. | A | A | A | A |
| 74. | A | A | A | A |
| 75. | A | A | A | A |
| 76. | D | C | C | D |
| 77. | D | D | C | C |
| 78. | A | A | A | A |
| 79. | A | A | A | A |
| 80. | A | A | A | A |
| 81. | A | A | A | A |
| 82. | B | A | A | A |
| 83. | A | A | A | A |
| 84. | A | A | A | A |
| 85. | A | A | A | A |
| 86. | A | A | A | A |
| 87. | D | D | D | D |
| 88. | A | A | A | A |
| 89. | A | A | A | A |
| 90. | A | A | A | A |
| 91. | A | A | A | A |
| 92. | B | B | A | B |
| 93. | A | A | A | A |
| 94. | B | C | A | B |
| 95. | A | A | A | A |
| 96. | A | A | A | A |
| 97. | A | A | A | A |
| 98. | A | A | A | A |
| 99. | A | A | A | A |
| 100. | A | A | A | A |
| 101. | B | C | A | B |
| 102. | A | A | A | A |
| 103. | C | B | A | B |
| 104. | D | D | D | D |
| 105. | A | A | A | A |
| 106. | A | A | A | A |
| 107. | C | B | A | A |
| 108. | A | A | A | A |
| 109. | A | A | A | A |
| 110. | D | D | D | C |
| 111. | D | D | D | D |
| 112. | B | B | A | A |
| 113. | A | A | A | A |
| 114. | A | A | A | A |
| 115. | B | B | A | A |
| 116. | B | A | A | A |
| 117. | D | D | D | D |
| 118. | D | D | D | D |
| 119. | A | A | A | A |
| 120. | A | A | A | A |
| 121. | A | A | A | A |
| 122. | A | A | A | A |
| 123. | A | A | A | A |
| 124. | B | B | A | A |
| 125. | A | A | A | A |
| 126. | A | A | A | A |
| 127. | A | A | A | A |
| 128. | A | A | A | A |
| 129. | A | A | A | A |
| 130. | D | C | D | C |
| 131. | D | D | D | D |
| 132. | A | A | A | A |
| 133. | A | A | A | A |
| 134. | D | D | D | D |
| 135. | C | A | B | A |
| 136. | A | A | A | A |
| 137. | A | A | A | A |
| 138. | B | A | A | A |
| 139. | D | D | D | D |
| 140. | B | A | A | A |
| 141. | A | A | A | A |
| 142. | A | A | A | A |
| 143. | A | A | A | A |
| 144. | A | A | A | A |
| 145. | D | C | C | C |
| 146. | A | A | A | A |
| 147. | A | A | A | A |
| 148. | A | A | A | A |
| 149. | B | A | A | A |
| 150. | C | A | A | B |
| 151. | D | D | D | D |
| 152. | D | D | D | D |
| 153. | C | B | A | A |
| 154. | C | A | A | A |
| 155. | B | A | A | A |
| 156. | A | A | A | A |
| 157. | C | A | A | A |
| 158. | A | A | A | A |
| 159. | A | A | A | A |
| 160. | A | A | A | A |
| 161. | A | A | A | A |
| 162. | A | A | A | A |
| 163. | A | A | A | A |
| 164. | A | A | A | A |
| 165. | A | A | A | A |
| 166. | A | A | A | A |
| 167. | A | A | A | A |
| 168. | A | A | A | A |
| 169. | C | A | A | A |
| 170. | B | A | A | A |
| 171. | B | B | A | B |
| 172. | C | A | A | A |
| 173. | C | B | A | A |
| 174. | A | A | A | A |
| 175. | A | A | A | A |
| 176. | A | A | A | A |
| 177. | C | A | A | A |
| 178. | A | A | A | A |
| 179. | C | B | B | B |
| 180. | B | A | A | A |
| 181. | A | A | A | A |
| 182. | A | A | A | A |
| 183. | A | A | A | A |
| 184. | C | A | A | A |
| 185. | A | A | A | A |
| 186. | A | A | A | A |
| 187. | A | A | A | A |
| 188. | A | A | A | A |
| 189. | A | A | A | A |
| 190. | A | A | A | A |
| 191. | A | A | A | A |
| 192. | A | A | A | A |
| 193. | A | A | A | A |
| 194. | A | A | A | A |
| 195. | A | A | A | A |
| 196. | A | A | A | A |
| 197. | C | C | B | C |
| 198. | C | B | B | C |
| 199. | B | A | A | A |
| 200. | A | A | A | A |
| 201. | A | A | A | A |
| 202. | A | A | A | A |
| 203. | A | A | A | A |
| 204. | A | A | A | A |
| 205. | A | A | A | A |
| 206. | A | A | A | A |
| 207. | A | A | A | A |
| 208. | A | A | A | A |
| 209. | D | D | D | D |
| 210. | A | A | A | A |
| 211. | A | A | A | A |
| 212. | A | A | A | A |
| 213. | D | D | D | D |
| 214. | A | A | A | A |
| 215. | A | A | A | A |
| 216. | A | A | A | A |
| 217. | D | D | D | D |
| 218. | A | A | A | A |
| 219. | D | C | B | C |
| 220. | C | C | B | B |
| 221. | D | D | D | D |
| 222. | D | D | D | D |
| 223. | A | A | A | A |
| 224. | C | C | B | B |
| 225. | D | D | C | C |
| 226. | A | A | A | A |
| 227. | B | A | A | A |
| 228. | A | A | A | D |

A = 0.1-100 nM
B = >100-250 nM
C = >250-1000 nM
D = >1000 nM

TABLE 3
FGFR specific activity in Cancer Cell Viability Assays

| | Cancer Cell Viability Assays | | | |
|---|---|---|---|---|
| | RT112/84 | UM-UC-14 | KG-1 | SNU16 |
| | FGFR3 | FGFR3 | FGFR1 | FGFR2 |
| Example | (IC50 nM) | (IC50 nM) | (IC50 nM) | (IC50 nM) |
| 1 | B | | | |
| 2 | A | A | A | |
| 7 | D | D | C | |
| 8 | A | A | A | |
| 9 | A | A | A | |
| 10 | A | A | A | |
| 19 | A | A | A | |
| 22 | A | A | A | |
| 23 | A | A | A | |
| 24 | A | A | A | |
| 25 | A | A | A | |
| 26 | B | A | A | |
| 27 | A | A | A | |
| 28 | D | A | A | |
| 30 | A | A | A | |
| 32 | B | | | |
| 34 | A | A | A | |
| 35 | A | A | A | |
| 36 | B | A | | |
| 38 | A | | | |
| 40 | A | A | A | |
| 41 | C | | | |
| 42 | A | A | A | |
| 43 | | B | | |
| 44 | A | A | A | A |
| 49 | A | A | A | A |
| 50 | A | | | |
| 53 | | A | A | A |
| 54 | A | A | A | A |
| 55 | A | A | A | |
| 57 | A | A | A | |
| 61 | A | A | A | A |
| 64 | A | A | A | A |
| 67 | A | A | A | |
| 68 | A | A | | |
| 69 | A | A | | |
| 70 | C | | | |
| 76 | | C | | |
| 77 | | D | | |
| 78 | A | | | A |
| 80 | | A | | |
| 81 | A | A | A | |
| 85 | A | A | A | |
| 89 | | A | | A |
| 90 | | A | | A |
| 97 | | | | A |
| 100 | | | | A |
| 102 | | | | A |
| 103 | | | | A |
| 106 | A | A | A | A |
| 107 | | | | A |
| 120 | | | | A |
| 121 | A | A | | A |
| 122 | | | | A |
| 123 | | | | A |
| 124 | | | | A |
| 125 | | | | A |
| 126 | | | | A |
| 127 | | | | A |
| 128 | | | | A |
| 136 | A | A | | A |
| 144 | | | | A |
| 146 | | A | A | A |
| 147 | | | | A |
| 148 | | | | A |
| 149 | | | | A |
| 150 | | | | A |
| 153 | | | | A |
| 154 | | | | A |
| 156 | | | | A |
| 158 | | | | A |
| 160 | A | A | | A |
| 162 | | A | | A |
| 163 | A | A | | A |
| 164 | | | | A |
| 165 | | | | A |
| 167 | A | A | | A |
| 175 | | | | A |
| 176 | | | | A |
| 178 | | | | A |
| 180 | | | | A |
| 181 | A | A | | A |
| 182 | | | | A |
| 183 | | | | A |
| 184 | | A | | A |
| 185 | A | | | A |
| 186 | A | | | A |
| 187 | A | | | A |
| 188 | A | A | | |
| 189 | A | A | | |
| 190 | A | A | | |
| 201 | | | | A |
| 207 | | A | | |
| 208 | | | | A |
| 211 | | | | A |
| 214 | | | | A |
| 215 | | | | A |

A = 0.1-100 nM
B = >100-250 nM
C = >250-1000 nM
D = >1000 nM

In some embodiments, the disclosure is directed to the following aspects: Aspect 1. A compound of formula 1:

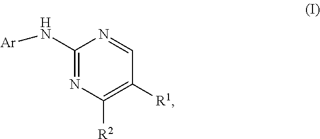

or a pharmaceutically acceptable salt thereof,
wherein:
   Ar is a 6-10-membered aryl group or a 5-10-membered heteroaryl group, each optionally substituted;
   R¹ is H, F, Cl, Br, C₁₋₆alkyl, CN, C₁₋₆haloalkyl, —C(O)NH₂, —C(O)NH(C₁-6alkyl), or —C(O)N(C₁₋₆alkyl)₂;
   R² is a 9-membered bicyclic heteroaryl comprising a 6-5-fused ring system wherein the 5-membered ring contains at least one nitrogen atom and the 6 membered ring is substituted with at least one E, wherein E is an electrophilic moiety that is capable of reacting with a nucleophile so as to form a covalent bond between an atom of the nucleophile and an atom of the electrophilic moiety.

Aspect 2. The compound according to Aspect 1, wherein R² is

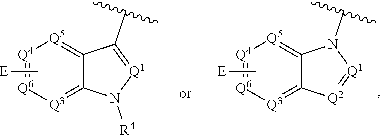

wherein
Q¹ is N or C—H;
Q² is N or C-R^{3A},
R^{3A} is H, C₁-C₆alkyl, —CN, —C₁-C₆haloalkyl, halo, —CON(R)₂, —NR₂, cycloalkyl, —CH₂cycloalkyl, —COR, —CH(OH)R, —CO-cycloalkyl, —CH(OH)cycloalkyl, —SR, or —SO₂R;
   each R is independently H, or C₁-C₆alkyl;
Q³, Q⁴, Q⁵, and Q⁶ are each independently N or C—R^{3B};
each R^{3B} is independently H, —C₁-C₆alkyl, —C₁-C₆haloalkyl, —CN, C₃-C₇cycloalkyl, or E, provided that at least one of Q³, Q⁴, Q⁵, and Q⁶ is C-E, and
R⁴ is C₁-C₆alkyl, C₁-C₆haloalkyl, C₃-C₇cycloalkyl, or SO₂(C₁-C₆alkyl).

Aspect 3. The compound according to Aspect 2, wherein R² is

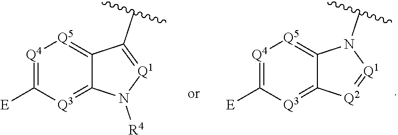

Aspect 4. The compound according to Aspect 1, wherein the compound of formula I is a compound of formula IA:

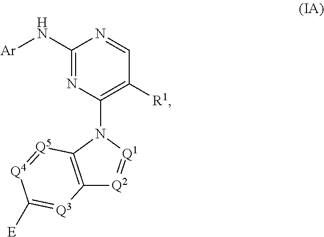

or a pharmaceutically acceptable salt thereof, wherein
Q¹ is N or C—H;
Q² is N or C—R^{3A};
   R^{3A} is H, C₁-C₆alkyl, —CN, —C₁-C₆haloalkyl, halo, —CON(R)₂, —NR₂, cycloalkyl, —CH₂cycloalkyl, —COR, —CH(OH)R, —CO-cycloalkyl, —CH(OH)cycloalkyl, —SR, or —SO₂R;
   each R is independently H, or C₁-C₆alkyl;
Q³, Q⁴, and Q⁵ are each independently N or C—R^{3B}; and
   each R^{3B} is independently H, —C₁-C₆alkyl, —C₁-C₆haloalkyl, —CN, or C₃-C₇cycloalkyl.

Aspect 5. The compound according to Aspect 4, wherein
Q¹ is C—H;
Q² is C—R^{3A};
Q³ and Q⁴ are each C—R^{3B};
Q⁵ is N or C—R^{3B};
R^{3A} is H or —CH₃; and
each R^{3B} is independently H or —CH₃.

Aspect 6. The compound according to Aspect 4, wherein
Q¹ is N;
Q² is C—R^{3A};
Q³ and Q⁴ are each C—R^{3B};
Q⁵ is N or C—R^{3B};
R^{3A} is H or —CH₃; and
each R^{3B} is independently H or —CH₃.

Aspect 7. The compound according to Aspect 1, wherein the compound of formula I is a compound of formula IB:

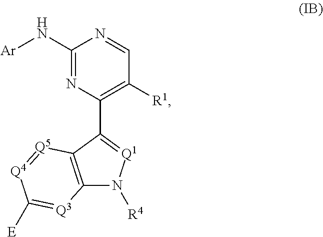

or a pharmaceutically acceptable salt thereof,
Q¹ is N or C—H;
Q³, Q⁴, and Q⁵ are each independently N or C—R^{3B};
   each R^{3B} is independently H, —C₁-C₆alkyl, —C₁-C₆haloalkyl, —CN, or C₃-C₇cycloalkyl; and
R⁴ is C₁-C₆alkyl, C₁-C₆haloalkyl, C₃-C₇cycloalkyl, or SO₂(C₁-C₆alkyl).

Aspect 8. The compound according to claim 7, wherein
Q¹ is C—H;
R⁴ is —CH₃
Q³, Q⁴, and Q⁵ are each independently N or C—R^{3B}; and
each R^{3B} is independently H or —CH₃.

Aspect 9. The compound according to Aspect 7, wherein
Q¹ is N;
R⁴ is —CH₃
Q³, Q⁴, Q¹ is N or C—R^{3B}; and
each R^{3B} is independently H or —CH₃.

Aspect 10. The compound according to anyone of the preceding Aspect, wherein R¹ is H, —Cl, —F, —CH₃, or —CN.

Aspect 11. The compound according to Aspect 10, wherein R¹ is —C₁.

Aspect 12. The compound according to Aspect 10, wherein R¹ is —CH₃.

Aspect 13. The compound according to Aspect 10, wherein R¹ is —H.

Aspect 14. The compound according to Aspect 10, wherein R¹ is —F.

Aspect 15. The compound according to Aspect 10, wherein R¹ is —CN.

Aspect 16. The compound according to any one of the preceding Aspects, wherein Ar is an optionally substituted 6-10-membered aryl group.

Aspect 17. The compound according to Aspect 16, wherein the optionally substituted 6-10-membered aryl group is an optionally substituted phenyl group.

Aspect 18. The compound according to Aspect 17, wherein the phenyl group is substituted with one or more optionally substituted C₁-C₆alkyl, optionally substituted C₃-C₆cycloalkyl, optionally substituted 4-6-membered heterocycloalkyl, —N(C₁-C₆alkyl)(optionally substituted C₁-C₆alkyl), —O(optionally substituted C₁-C₆alkyl), —CN, or halogen.

Aspect 19. The compound according to any one of Aspects 1 to 15, wherein Ar is an optionally substituted 5-10-membered heteroaryl group.

Aspect 20. The compound according to Aspect 19, wherein the 5-10-membered heteroaryl group is a pyridinyl, a pyrazolyl, a triazolyl, an imidazolyl, a pyrazolopyrimidine, or a triazolopyridine.

Aspect 21. The compound according to any one of Aspects 19 or 20, wherein the 5-10-membered heteroaryl group is substituted with one or more optionally substituted C₁-C₆alkyl, optionally substituted C₃-C₆cycloalkyl, optionally substituted 4-6-membered heterocycloalkyl, —N(C₁-C₆alkyl)(optionally substituted C₁-C₆alkyl), —O(optionally substituted C₁-C₆alkyl), —CN, or halogen.

Aspect 22. The compound according to Aspect 18 or Aspect 21, wherein the optionally substituted C₁-C₆alkyl is —CH₃, —CH₂CH₃, isopropyl, —CH₂CF₃, —CHF₂, —CF₃, —CH₂-cyclopropyl, —CH₂-(optionally substituted 5 or 6-membered heterocycloalkyl), or —CH₂CH₂OH.

Aspect 23. The compound according to Aspect 22, wherein the —CH₂-(optionally substituted 5 or 6-membered heterocycloalkyl) is

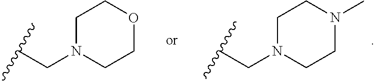

Aspect 24. The compound according to Aspect 18 or Aspect 21, wherein the optionally substituted C₃-C₆cycloalkyl is cyclopropyl or cyclobutyl.

Aspect 25. The compound according to Aspect 18 or Aspect 21, wherein the optionally substituted 4-6-membered heterocycloalkyl is

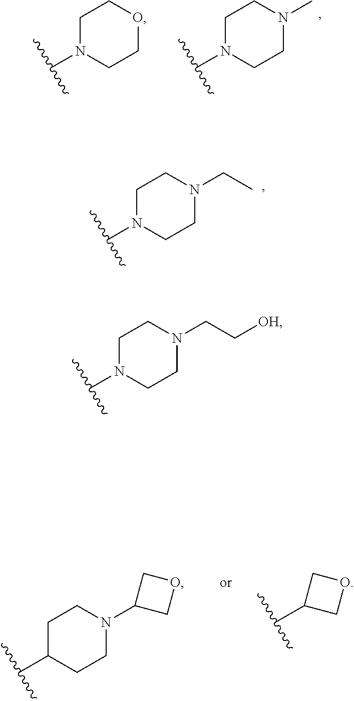

Aspect 26. The compound according to Aspect 18 or cla Aspect im 21, wherein the —N(C₁-C₆alkyl)(optionally substituted C₁-C₆alkyl) is

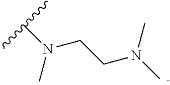 [SMILES: *N(C)CCN(C)C]

Aspect 27. The compound according to Aspect 18 or Aspect 21, wherein the —O (optionally substituted C₁-C₆alkyl) is —OCH₃, —OCH₂CH₃, or

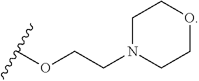 [SMILES: *OCCN1CCOCC1]

Aspect 28. The compound according to Aspect 18 or Aspect 21, wherein the halogen is —F.

Aspect 29. The compound according to Aspect 16, wherein Ar is

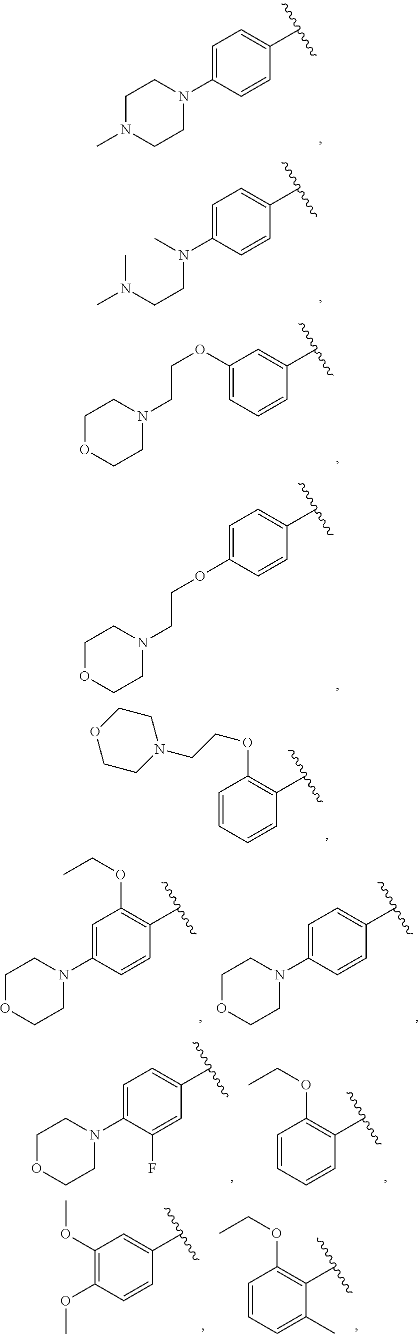

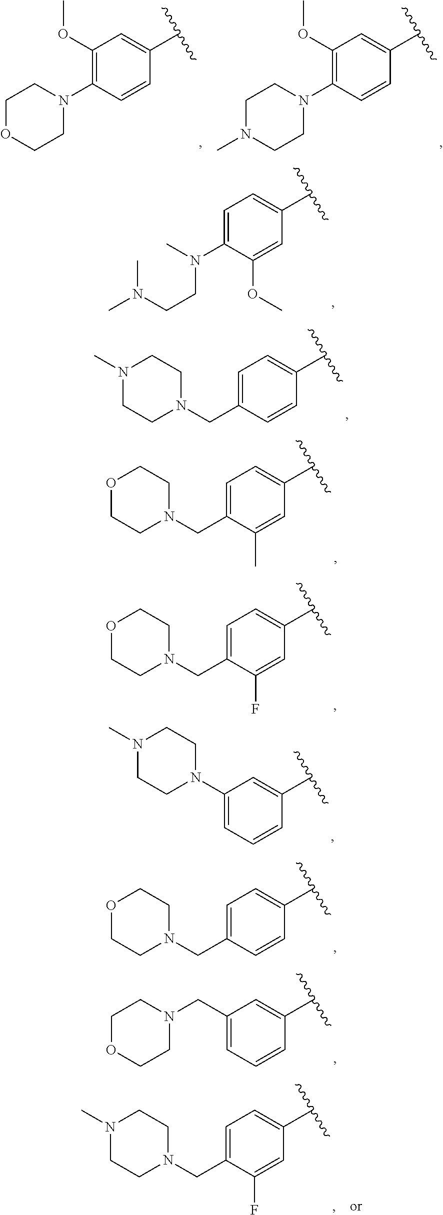

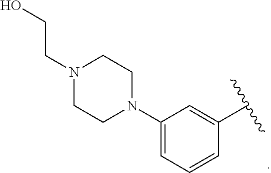 [SMILES: *c1cccc(N2CCN(CCO)CC2)c1]

Aspect 30. The compound according to Aspect 19, wherein Ar is

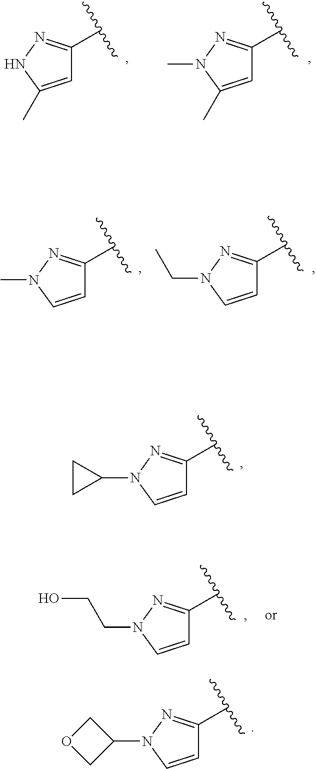

Aspect 31. The compound according to Aspect 19, wherein Ar is

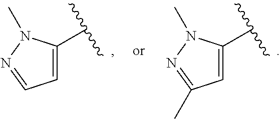

Aspect 32. The compound according to Aspect 19, wherein Ar is

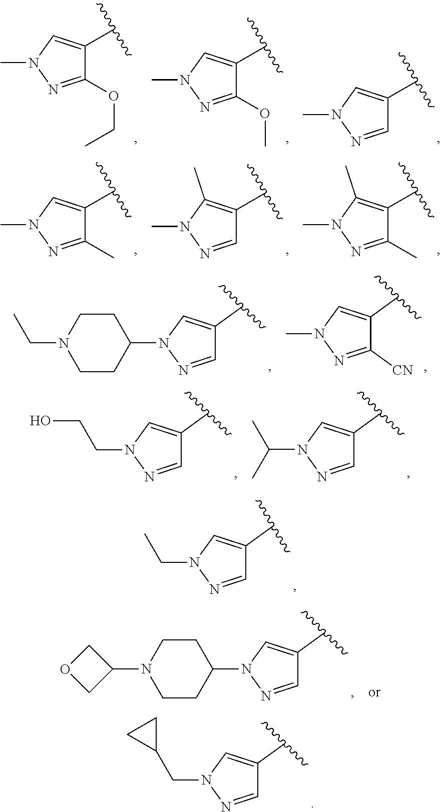

Aspect 33. The compound according to Aspect 19, wherein Ar is

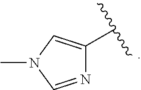 [SMILES: *c1cn(C)cn1]

Aspect 34. The compound according to Aspect 19 wherein Ar is

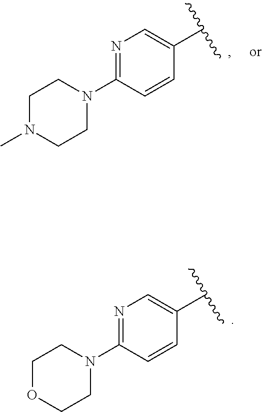 [SMILES: *c1ccc(N2CCN(C)CC2)nc1 ; *c1ccc(N2CCOCC2)nc1]

Aspect 35. The compound according to Aspect 19, wherein Ar is

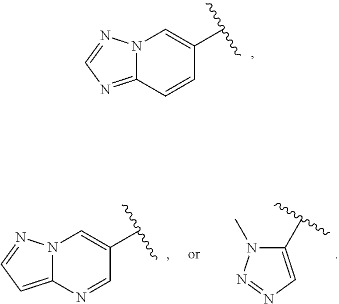

Aspect 36. The compound according to Aspect 49, wherein the compound of formula IA is a compound of formula IA-1

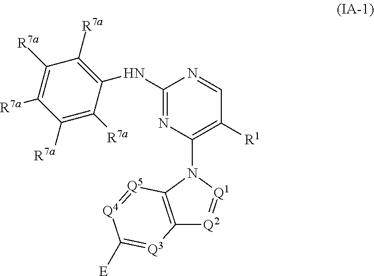

wherein each R^{7a} is independently H, —CH₃, —CH₂CH₃, isopropyl, —CH₂CF₃, —CHF₂, —CF₃, —CH₂-cyclopropyl, —CH₂CH₂OH,

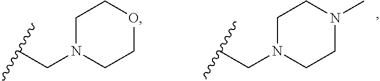

cyclopropyl, cyclobutyl,

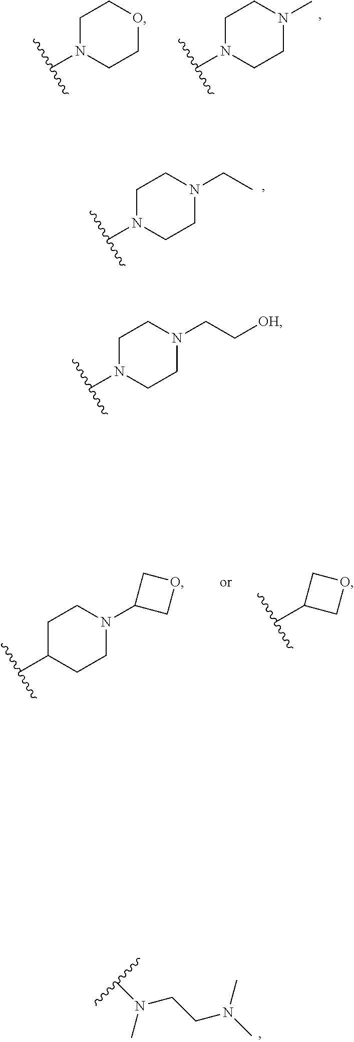

OCH₃, —OCH₂CH₃,

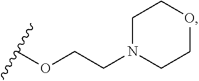 [SMILES: *OCCN1CCOCC1]

### —CN, or —F.

Aspect 37. The compound according to Aspect 4, wherein the compound of formula IA is a compound of formula IA-2:

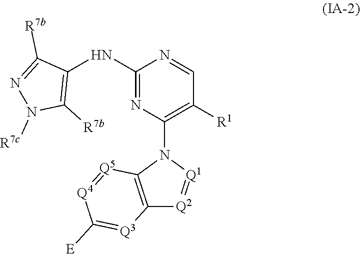

wherein each R^{7b} is independently H, —CH₃, —CH₂CH₃, isopropyl, —CH₂CF₃, —CHF₂, —CF₃, —CH₂-cyclopropyl, —CH₂CH₂OH,

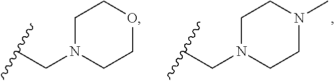

cyclopropyl, cyclobutyl,

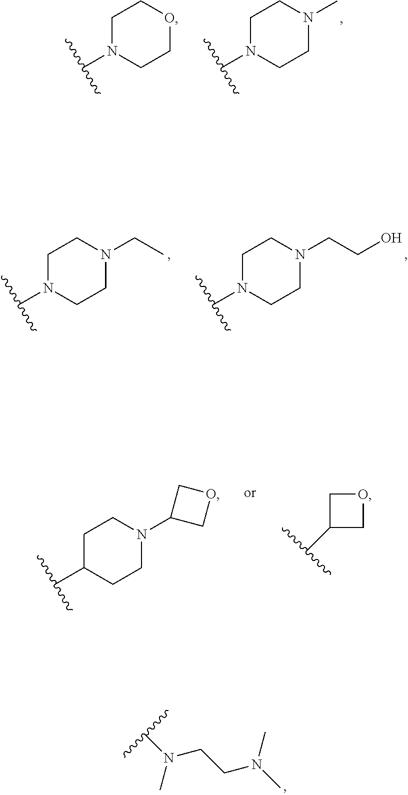

—OCH₃, —OCH₂CH₃,

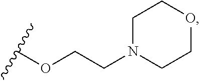 [SMILES: *OCCN1CCOCC1]

### —CN, or —F;

and R^{7c} is H, —CH₃, —CH₂CH₃, isopropyl, —CH₂CF₃, —CHF₂, —CF₃, —CH₂-cyclopropyl, —CH₂CH₂OH,

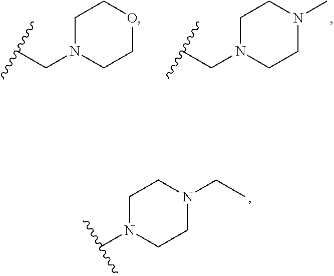

cyclopropyl, cyclobutyl,

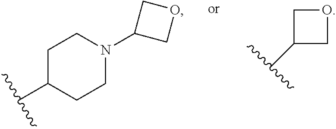 [SMILES: *C1CCN(C2COC2)CC1 ; "or" ; *C1COC1]

Aspect 38. The compound according to Aspect 4, wherein the compound of formula IA is a compound of formula IA-3:

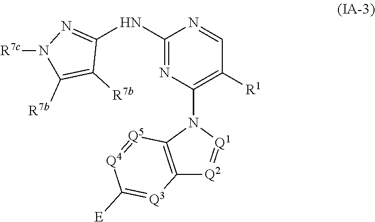

wherein each R^{7b} is independently H, —CH₃, —CH₂CH₃, isopropyl, —CH₂CF₃, —CHF₂, —CF₃, —CH₂-cyclopropyl, —CH₂CH₂OH

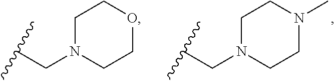

cyclopropyl, cyclobutyl,

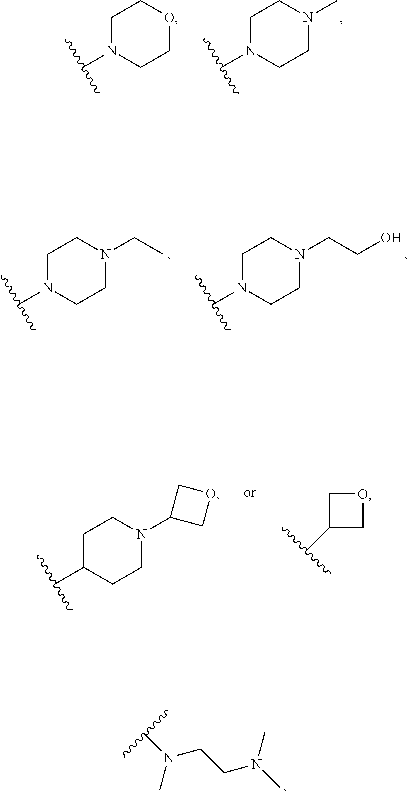

—OCH₃, —OCH₂CH₃,

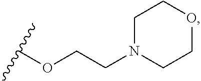 [SMILES: *OCCN1CCOCC1]

### —CN, or —F:

and R^{7c} is H, —CH₃, —CH₂CH₃, isopropyl, —CH₂CF₃—CHF₂, —CF₃, —CH₂-cyclopropyl, —CH₂CH₂OH,

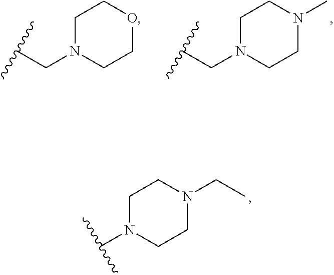

cyclopropyl, cyclobutyl,

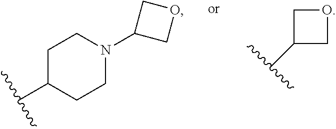 [SMILES: *C1CCN(C2COC2)CC1 ; "or" ; *C1COC1]

Aspect 39. The compound according to Aspect 4, wherein the compound of formula IA is a compound of formula IA-4:

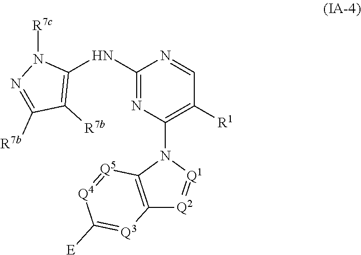

wherein each R^{7b} is independently H—CH₃, —CH₂CH₃, isopropyl, —CH₂CF₃, —CHF₂, —CF₃, —CH₂-cyclopropyl, —CH₂CH₂OH,

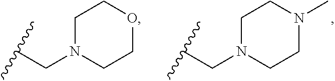

cyclopropyl, cyclobutyl,

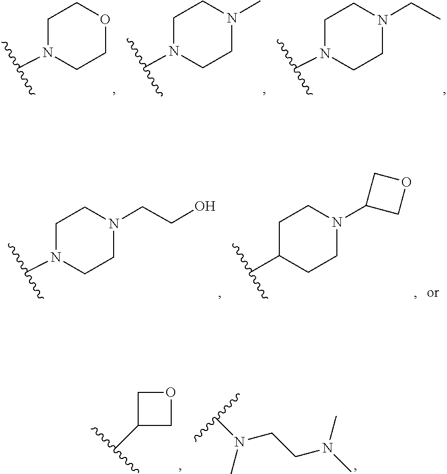

—OCH₃, —OCH₂CH₃,

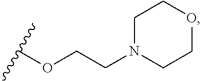 [SMILES: *OCCN1CCOCC1]

—CN, or —F; and R^{7c} is H—CH₃, —CH₂CH₃, isopropyl, —CH₂CF₃—CHF₂, —CF₃, —CH₂-cyclopropyl, —CH₂CH₂OH,

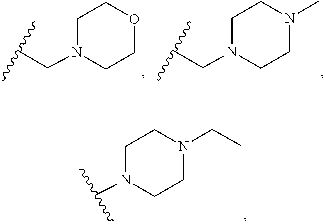

cyclopropyl, cyclobutyl,

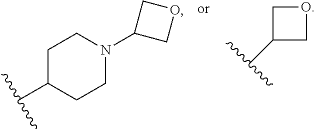 [SMILES: *C1CCN(C2COC2)CC1 ; *C1COC1]

Aspect 40. The compound according to Aspect 4, wherein the compound of formula IA is a compound of formula IA-5:

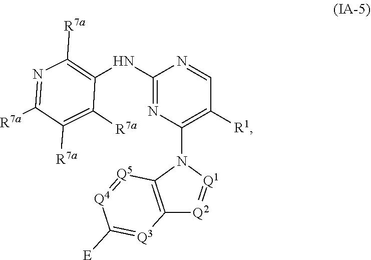

wherein each R^{7a} is independently H—CH₃—CH₂CH₃, isopropyl, —CH₂CF₃, —CHF₂, —CF₃, —CH₂-cyclopropyl, —CH₂CH₂OH,

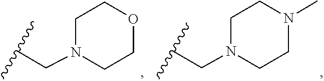

cyclopropyl, cyclobutyl,

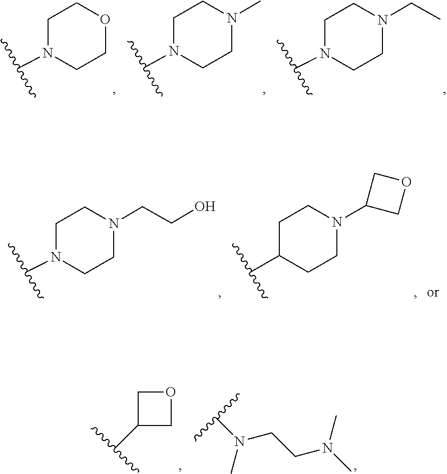

—OCH₃, —OCH₂CH₃,

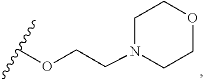 [SMILES: *OCCN1CCOCC1]

### —CN, or —F.

Aspect 41. The compound according to Aspect 4, wherein the compound of formula IA is a compound of formula IA-6:

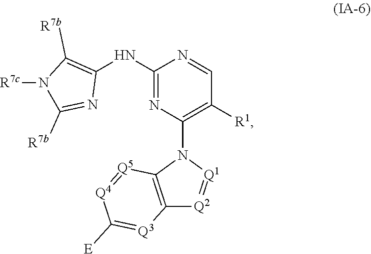

wherein each R^{7b} is independently H, —CH₃, —CH₂CH₃, isopropyl, —CH₂CF₃, —CHF₂, —CF₃, —CH₂-cyclopropyl, —CH₂CH₂OH,

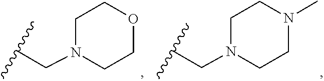

cyclopropyl, cyclobutyl,

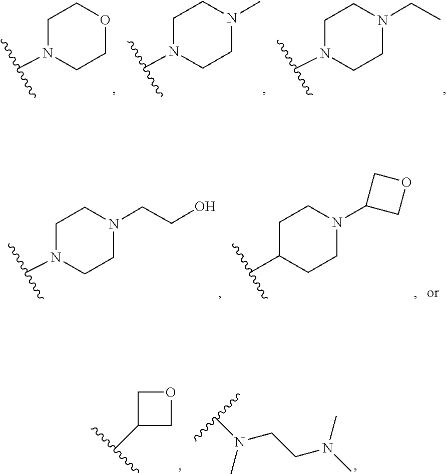

—OCH₃, —OCH₂CH₃,

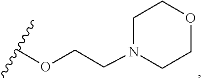 [SMILES: *OCCN1CCOCC1]

### —CH, or —F;

and R^{7c} is H—CH₃, —CH₂CH₃, isopropyl, —CH₂CF₃—CHF₂, —CF₃, —CH₂-cyclopropyl, —CH₂CH₂OH,

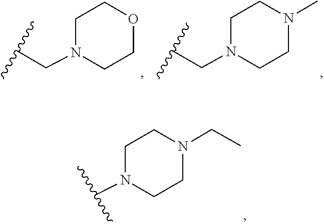

cyclopropyl, cyclobutyl,

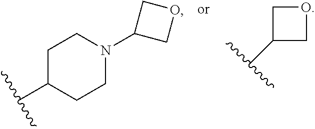 [SMILES: *C1CCN(C2COC2)CC1 ; *C1COC1]

Aspect 42. The compound according to any one of Aspects 36-41, wherein Q¹ is C—H; Q² is C—R^{3A}; Q³ and Q⁴ are each C—R^{3B}; Q⁵ is N or C—R^{3B}; R^{3A} is H or —CH₃; and each R^{3B} is independently H or —CH₃.

Aspect 43. The compound according to any one of Aspects 36-41, wherein Q¹ is C—H; Q² is C—R^{3A}; Q³ and Q⁴ are each C—R^{3B}; Q⁵ is C—R^{3B}; R^{3A}—CH₃; and each R^{3B} is H.

Aspect 44. The compound according to any one of Aspects 36-41, wherein Q¹ is N; Q² is C—R^{3A}; Q³ and Q⁴ are each C—R^{3B}; Q⁵ is N or C—R^{3B}; R^{3A} is H or —CH₃; and each R^{3B} is independently H or —CH₃.

Aspect 45. The compound according to Aspect 7, wherein the compound of formula IB is a compound of formula IB-1:

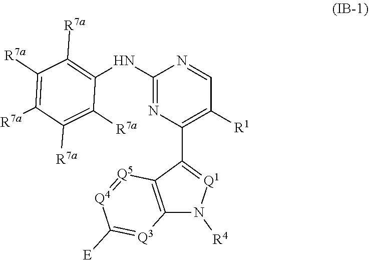

wherein each R^{7a} is independently H, —CH₃, —CH₂CH₃, isopropyl, —CH₂CF₃, —CHF₂, —CF₃, —CH₂-cyclopropyl, —CH₂CH₂OH,

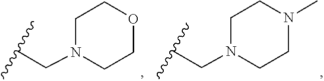

cyclopropyl, cyclobutyl,

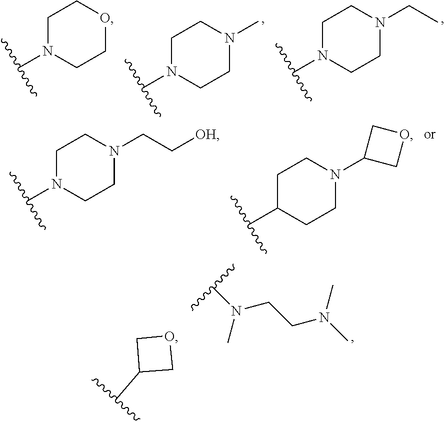

—OCH₃, —OCH₂CH₃,

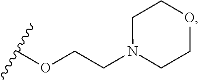 [SMILES: *OCCN1CCOCC1]

### —CN, or —F.

Aspect 46. The compound according to Aspect 7, wherein the compound of formula IB is a compound of formula IB-2:

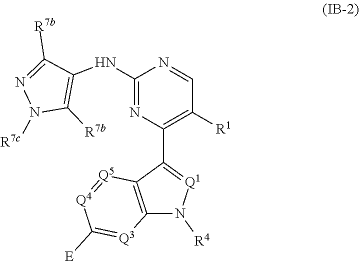

wherein each R^{7b} is independently H, —CH₃—CH₂CH₃, isopropyl, —CH₂CF₃, —CHF₂, —CF₃, —CH₂-cyclopropyl, —CH₂CH₂OH,

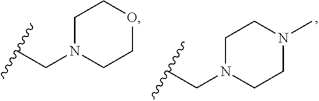

cyclopropyl, cyclobutyl,

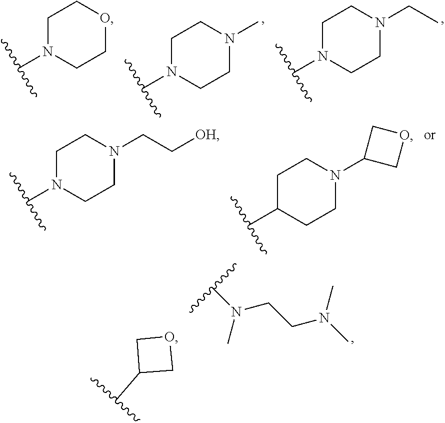

—OCH₃, —OCH₂CH₃,

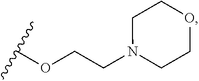 [SMILES: *OCCN1CCOCC1]

### —F;

and R^{7c} is H, —CH₃, —CH₂CH₃, isopropyl, —CH₂CF₃, —CHF₂, —CF₃, —CH₂-cyclopropyl, —CH₂CH₂OH,

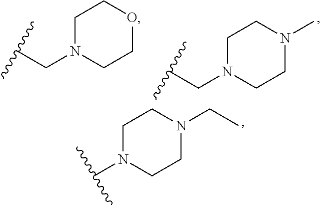

cyclopropyl, cyclobutyl,

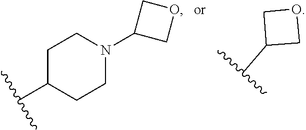 [SMILES: *C1CCN(C2COC2)CC1 ; "or" ; *C1COC1]

Aspect 47. The compound according to Aspect 7, wherein the compound of formula IB is a compound of formula IB-3:

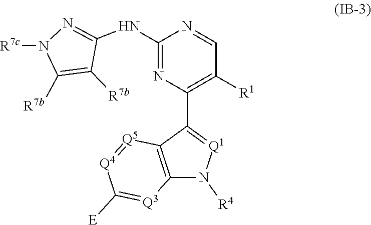

wherein each R^{7b} is independently H, —CH₃—CH₂CH₃, isopropyl, —CH₂CF₃, —CHF₂, —CF₃, —CH₂-cyclopropyl, —CH₂CH₂OH,

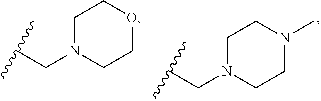

cyclopropyl, cyclobutyl,

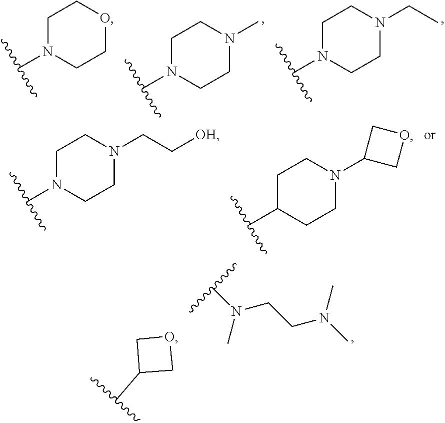

—OCH₃, —OCH₂CH₃,

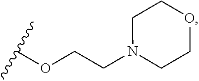 [SMILES: *OCCN1CCOCC1]

### —CN, or —F;

and R^{7c} is H, —CH₃, —CH₂CH₃, isopropyl, —CH₂CF₃, —CHF₂, —CF₃, —CH₂-cyclopropyl, —CH₂CH₂OH,

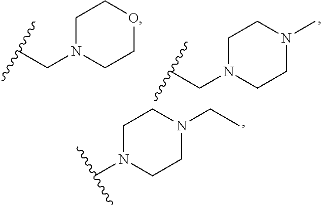

cyclopropyl, cyclobutyl,

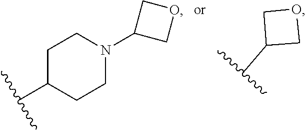 [SMILES: *C1CCN(C2COC2)CC1 ; "or" ; *C1COC1]

Aspect 48. The compound according to Aspect 7, wherein the compound of formula IB is a compound of formula IB-4:

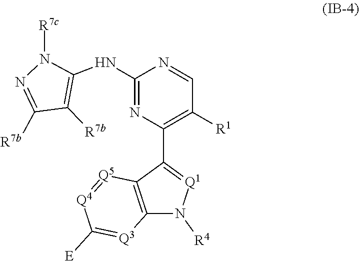

wherein each R^{7b} is independently H, —CH₃, —CH₂CH₃, isopropyl, —CH₂CF₃, —CHF₂, —CF₃, —CH₂-cyclopropyl, —CH₂CH₂OH,

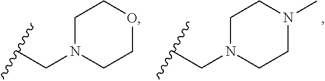

cyclopropyl, cyclobutyl,

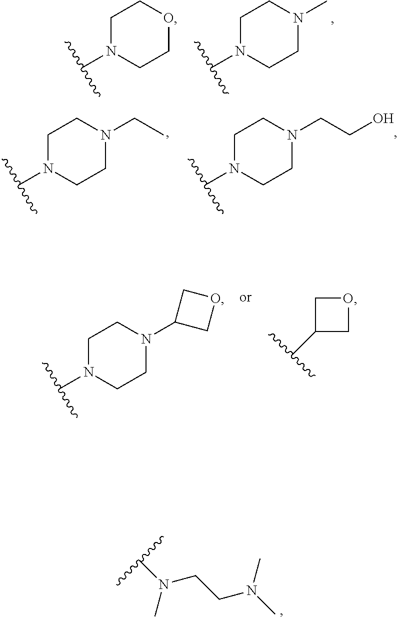

—OCH₃, —OCH₂CH₃,

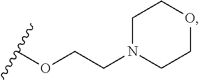 [SMILES: *OCCN1CCOCC1]

### —CN, or —F;

and R^{7c} is H, —CH₃, —CH₂CH₃, isopropyl, —CH₂CF₃, —CHF₂, —CF₃, —CH₂-cyclopropyl, —CH₂CH₂OH,

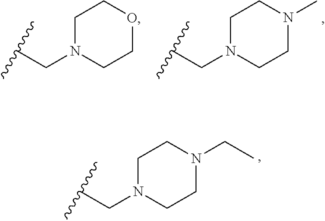

cyclopropyl, cyclobutyl,

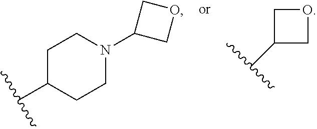 [SMILES: *C1CCN(C2COC2)CC1 ; *C1COC1]

Aspect 49. The compound according to Aspect 7, wherein the compound of formula IB is a compound of formula IB-5:

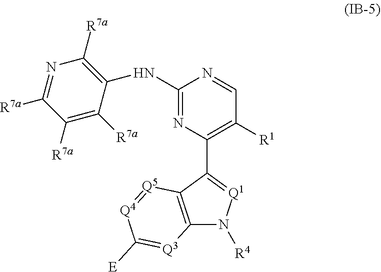

wherein each R^{7a} is independently H, —CH₃, —CH₂CH₃, isopropyl, —CH₂CF₃, —CHF₂, —CF₃, —CH₂-cyclopropyl, —CH₂CH₂OH,

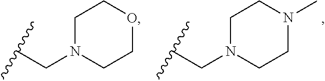

cyclopropyl, cyclobutyl,

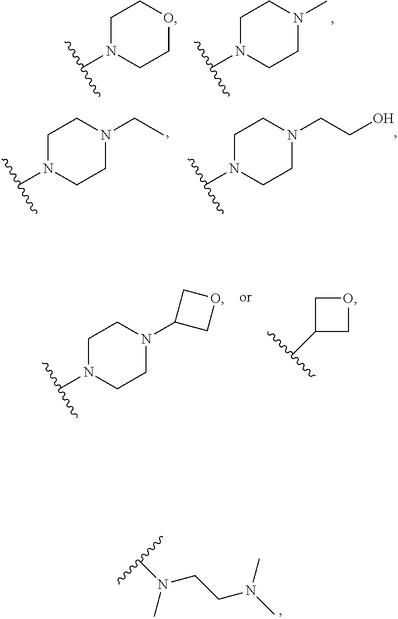

—OCH₃, —OCH₂CH₃,

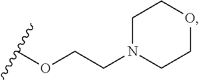 [SMILES: *OCCN1CCOCC1]

### —CN, or —F.

Aspect 50. The compound according to Aspect 7, wherein the compound of formula IB is a compound of formula IB-6:

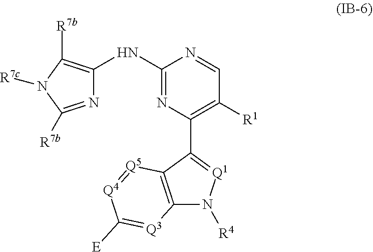

wherein each R^{7b} is independently H, —CH₃, —CH₂CH₃, isopropyl, —CH₂CF₃, —CHF₂, —CF₃, —CH₂-cyclopropyl, —CH₂CH₂OH,

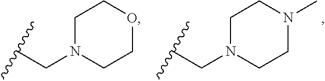

cyclopropyl, cyclobutyl,

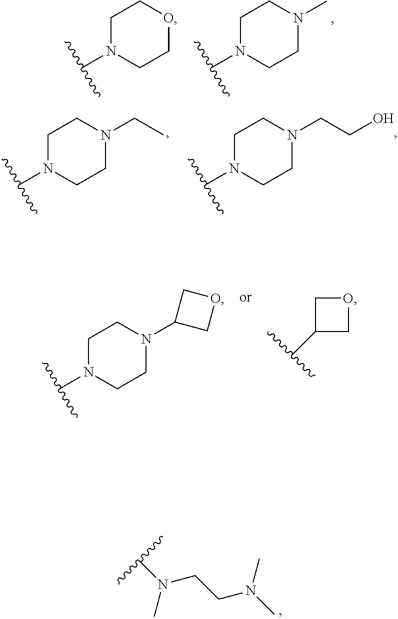

—OCH₃, —OCH₂CH₃,

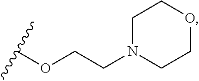 [SMILES: *OCCN1CCOCC1]

### —CN, or —F;

and R^{7c} is H, —CH₃, —CH₂CH₃, isopropyl, —CH₂CF₃—CHF₂, —CF₃, —CH₂-cyclopropyl, —CH₂CH₂OH,

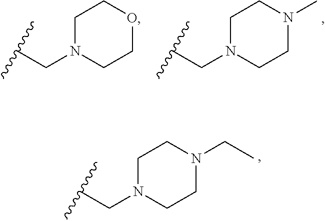

cyclopropyl, cyclobutyl,

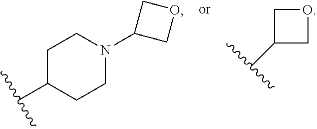 [SMILES: *C1CCN(C2COC2)CC1 ; *C1COC1]

Aspect 51. The compound according to any one of Aspects 45-50, wherein Q¹ is C—H; R⁴ is —CH₃; Q³, Q⁴, and Q⁵ are each independently N or C—R^{3B}; and each R^{3B} is independently H or —CH₃.

Aspect 52. The compound according to any one of Aspects 45-50, wherein Q¹ is N; R⁴ is —CH₃, each Q³, Q⁴, and Q⁵ is N or C—R^{3B}; and each R^{3B} is independently H or —CH₃.

Aspect 53. The compound according to any one of the preceding Aspects, wherein E is

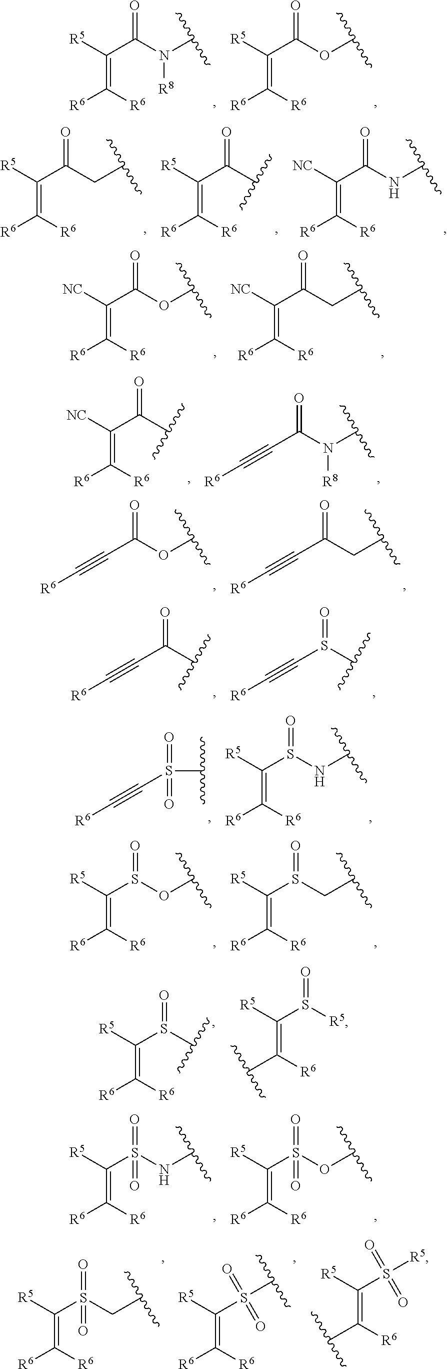

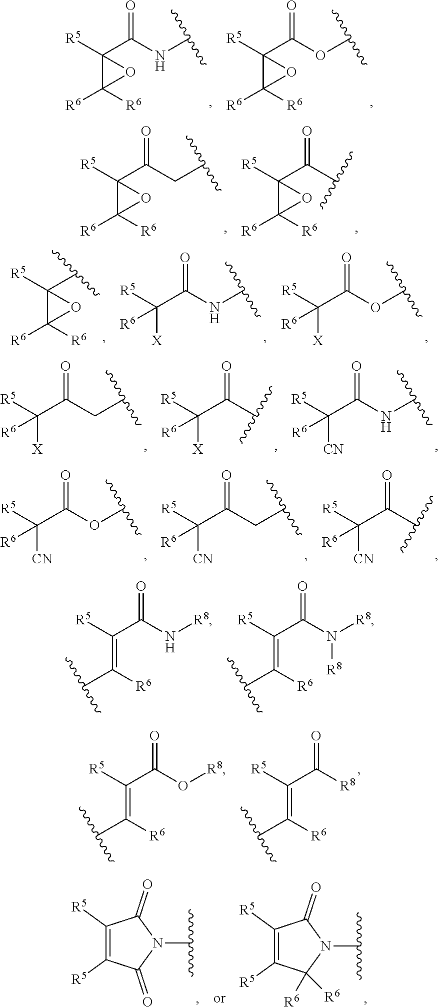

wherein each R⁵ and each R⁶ is independently H, optionally substituted C₁-C₆alkyl, or halogen; R⁸ is H or C₁-C₆alkyl; and X is halogen.

Aspect 54. The compound according to Aspect 53, wherein E is

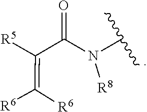

Aspect 55. The compound according to Aspect 54 wherein R⁵, R⁶, and R⁸ are each H.

Aspect 56. The compound according to Aspect 53, wherein E is

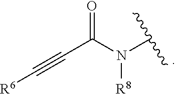

Aspect 57. The compound according to Aspect 56 wherein R⁶ is H and R⁸ is H.

Aspect 58. The compound according to Aspect 56 wherein R⁶ is —CH₃ and R⁸ is H.

Aspect 59. A pharmaceutically acceptable salt of a compound of any one of the preceding Aspects.

Aspect 60. A pharmaceutical composition comprising a compound of any one of the preceding Aspects and a pharmaceutically acceptable excipient.

Aspect 61. A method of treating cancer in a subject comprising administering to the subject a therapeutically effective amount of a compound of any one of Aspects 1 to 58, or a pharmaceutically acceptable salt thereof.

Aspect 62. The method of Aspect 61, wherein the cancer is urothelial carcinoma, breast carcinoma, endometrial adenocarcinoma, ovarian carcinoma, primary glioma, cholangiocarcinoma, gastric adenocarcinoma, non-small cell lung carcinoma, pancreatic exocrine carcinoma, oral, prostate, bladder, colorectal carcinoma, renal cell carcinoma, neuroendocrine carcinoma, myeloproliferative neoplasms, head and neck (squamous), melanoma, leiomyosarcoma, or a sarcoma.

Aspect 63. A compound of formula I:

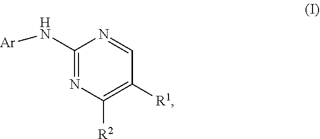

or a pharmaceutically acceptable salt thereof,
wherein:
   Ar is a 6-10-membered aryl group or a 5-10-membered heteroaryl group, each optionally substituted;
   R¹ is H, F, Cl, Br, C₁₋₆alkyl, CN, C₁₋₆haloalkyl, —C(O)NH₂, —C(O)NH(C₁₋₆alkyl), or —C(O)N(C₁₋₆alkyl)₂;
   R² is a 9-membered bicyclic heteroaryl comprising a 6-5-fused ring system wherein the 5-membered ring contains at least one nitrogen atom and the 6 membered ring is substituted with at least one E, wherein E is an electrophilic moiety that is capable of reacting with a nucleophile so as to form a covalent bond between an atom of the nucleophile and an atom of the electrophilic moiety.

Aspect 64. The compound according to aspect 63, wherein R² is

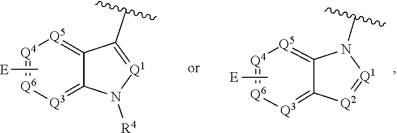

wherein
Q¹ is N or C—H;
Q² is N or C-R^{3A},
R^{3A} is H, C₁-C₆alkyl, —CN, —C₁-C₆haloalkyl, halo, —CON(R)₂, —NR₂, cycloalkyl, —CH₂cycloalkyl, —COR, —CH(OH)R, —CO-cycloalkyl, —CH(OH)cycloalkyl, —SR, or —SO₂R;
   each R is independently H, or C₁-C₆alkyl;
Q³, Q⁴, Q⁵, and Q⁶ are each independently N or C—R^{3B};
each R^{3B} is independently H, —C₁-C₆alkyl, —C₁-C₆haloalkyl, —CN, C₃-C₇cycloalkyl, or E, provided that at least one of Q³, Q⁴, Q⁵, and Q⁶ is C-E, and
R⁴ is C₁-C₆alkyl, C₁-C₆haloalkyl, C₃-C₇cycloalkyl, or SO₂(C₁-C₆alkyl).

Aspect 65. The compound according to aspect 64, wherein R² is

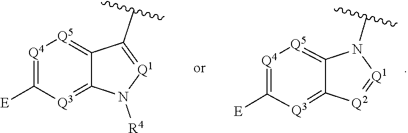

Aspect 66. The compound according to aspect 63, wherein the compound of formula I is a compound of formula IA:

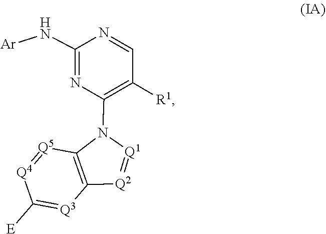

or a pharmaceutically acceptable salt thereof, wherein
Q¹ is N or C—H;
Q² is N or C—R^{3A};
   R^{3A} is H, C₁-C₆alkyl, —CN, —C₁-C₆haloalkyl, halo, —CON(R)₂, —NR₂, cycloalkyl, —CH₂cycloalkyl, —COR, —CH(OH)R, —CO-cycloalkyl, —CH(OH)cycloalkyl, —SR, or —SO₂R;
   each R is independently H, or C₁-C₆alkyl;
Q³, Q⁴, and Q⁵ are each independently N or C—R^{3B}; and
   each R^{3B} is independently H, —C₁-C₆alkyl, —C₁-C₆haloalkyl, —CN, or C₃-C₇cycloalkyl.

Aspect 67. The compound according to aspect 66, wherein
Q¹ is C—H;
Q² is C—R^{3A};
Q³ and Q⁴ are each C—R^{3B};
Q⁵ is N or C—R^{3B};
R^{3A} is H, —CH₃, —CH₂CH₃; and
each R^{3B} is independently H or —CH₃.

Aspect 68. The compound according to aspect 66, wherein
Q¹ is N;
Q² is C—R^{3A};
Q³ and Q⁴ are each C—R^{3B};
Q⁵ is N or C—R^{3B}.
R^{3A} is H, —CH₃, —CH₂CH₃; and
each R^{3B} is independently H or —CH₃.

Aspect 69. The compound according to aspect 63, wherein the compound of formula I is a compound of formula IB:

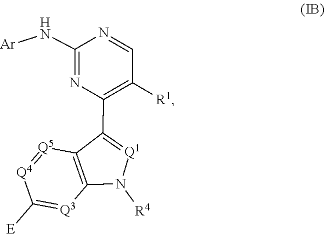

or a pharmaceutically acceptable salt thereof,
Q¹ is N or C—H;
Q³, Q⁴, and Q⁵ are each independently N or C—R^{3B};
   each R^{3B} is independently H, —C₁-C₆alkyl, —C₁-C₆haloalkyl, —CN, or C₃-C₇cycloalkyl; and
R⁴ is C₁-C₆alkyl, C₁-C₆haloalkyl, C₃-C₇cycloalkyl, or SO₂(C₁-C₆alkyl).

Aspect 70. The compound according to aspect 7, wherein
Q¹ is C—H;
R⁴ is —CH₃
Q³, Q⁴, and Q⁵ are each independently N or C—R^{3B}; and
each R^{3B} is independently H or —CH₃.

Aspect 71. The compound according to aspect 69, wherein
Q¹ is N;
R⁴ is —CH₃
Q³, Q⁴, Q⁵ is N or C—R^{3B}; and
each R^{3B} is independently H or —CH₃.

Aspect 72. The compound according to anyone of the preceding aspects, wherein R¹ is H, —Cl, —F, —CH₃, or —CN.

Aspect 73. The compound according to aspect 72, wherein R¹ is —C₁.

Aspect 74. The compound according to aspect 72, wherein R¹ is —CH₃.

Aspect 75. The compound according to aspect 72, wherein R¹ is —H.

Aspect 76. The compound according to aspect 72, wherein R¹ is —F.

Aspect 77. The compound according to aspect 72, wherein R¹ is —CN.

Aspect 78. The compound according to any one of aspects 63-77, wherein Ar is an optionally substituted 6-10-membered aryl group.

Aspect 79. The compound according to aspect 78, wherein the optionally substituted 6-10-membered aryl group is an optionally substituted phenyl group.

Aspect 80. The compound according to aspect 79, wherein the phenyl group is substituted with one or more optionally substituted C₁-C₆alkyl, optionally substituted C₃-C₆cycloalkyl, optionally substituted 4-6-membered heterocycloalkyl, —N(C₁-C₆alkyl)(optionally substituted C₁-C₆alkyl), —O(optionally substituted C₁-C₆alkyl), —CN, or halogen.

Aspect 81. The compound according to any one of aspects 63 to 77, wherein Ar is an optionally substituted 5-10-membered heteroaryl group.

Aspect 82. The compound according to aspect 81, wherein the 5-10-membered heteroaryl group is a pyridinyl, a pyrazolyl, a triazolyl, an imidazolyl, a pyrazolopyrimidine, or a triazolopyridine.

Aspect 83. The compound according to any one of aspects 81 or 82, wherein the 5-10-membered heteroaryl group is substituted with one or more optionally substituted C₁-C₆alkyl, optionally substituted C₃-C₆cycloalkyl, optionally substituted 4-6-membered heterocycloalkyl, —N(C₁-C₆alkyl)(optionally substituted C₁-C₆alkyl), —O(optionally substituted C₁-C₆alkyl), —CN, halogen, or —C(O)—O(optionally substituted C₁-C₆alkyl).

Aspect 84. The compound according to aspect 80 or aspect 83, wherein the optionally substituted C₁-C₆alkyl is —CH₃, —CH₂CH₃, isopropyl, —CH₂CF₃, —CHF₂, —CF₃, —CH₂-cyclopropyl, —CH₂-(optionally substituted 5 or 6-membered heterocycloalkyl), —CH₂CH₂OH, —CH₂CH(OH)CH₃, —CH₂C(OH)(CH₃)₂, —C(CN)(CH₃)₂, —CH₂CH₂CH₂OH, —CH₂CH₂OCH₃, —CH₂CH₂OCH₂CH₃, —CH₂CH₂OCH(CH₃)₂, CH₂CH(OCH₃)CH₃, —CH₂CHF₂, —CH₂CH₂N(CH₃)₂, —CH₂CH₂-(optionally substituted 5 or 6-membered heterocycloalkyl), or —CH₂-optionally substituted cyclopropyl.

Aspect 85. The compound according to aspect 84, wherein the —CH₂-(optionally substituted 5 or 6-membered heterocycloalkyl) is

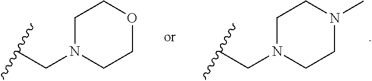

Aspect 86. The compound according to aspect 18 or aspect 21, wherein the optionally substituted C₃-C₆cycloalkyl is cyclopropyl or cyclobutyl.

Aspect 87. The compound according to aspect 80 or aspect 83, wherein the optionally substituted 4-6-membered heterocycloalkyl is

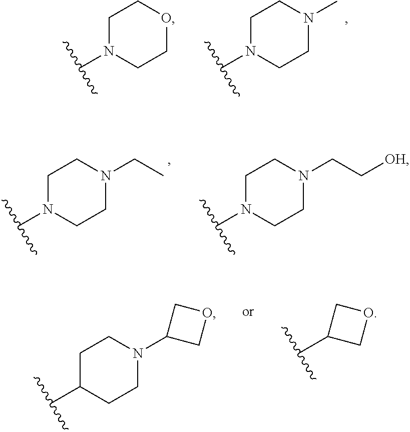

Aspect 88. The compound according to aspect 18 or aspect 21, wherein the —N(C₁-C₆alkyl) (optionally substituted C₁-C₆alkyl) is

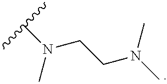 [SMILES: *N(C)CCN(C)C]

Aspect 89. The compound according to aspect 80 or aspect 83, wherein the —O(optionally substituted C₁-C₆alkyl) is —OCH₃, —OCH₂CH₃, or

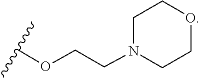 [SMILES: *OCCN1CCOCC1]

Aspect 90. The compound according to aspect 80 or aspect 83, wherein the halogen is —F.

Aspect 91. The compound according to aspect 78, wherein Ar is

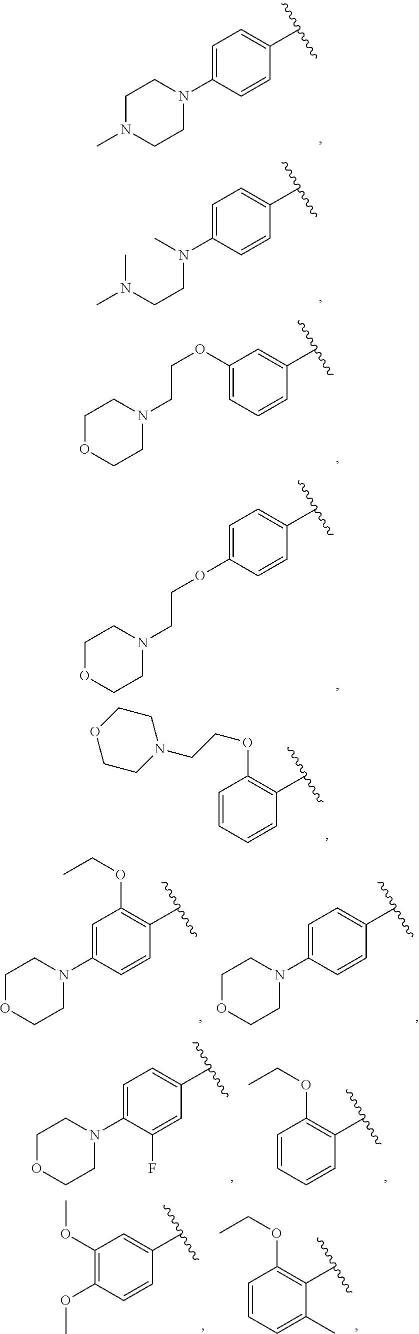

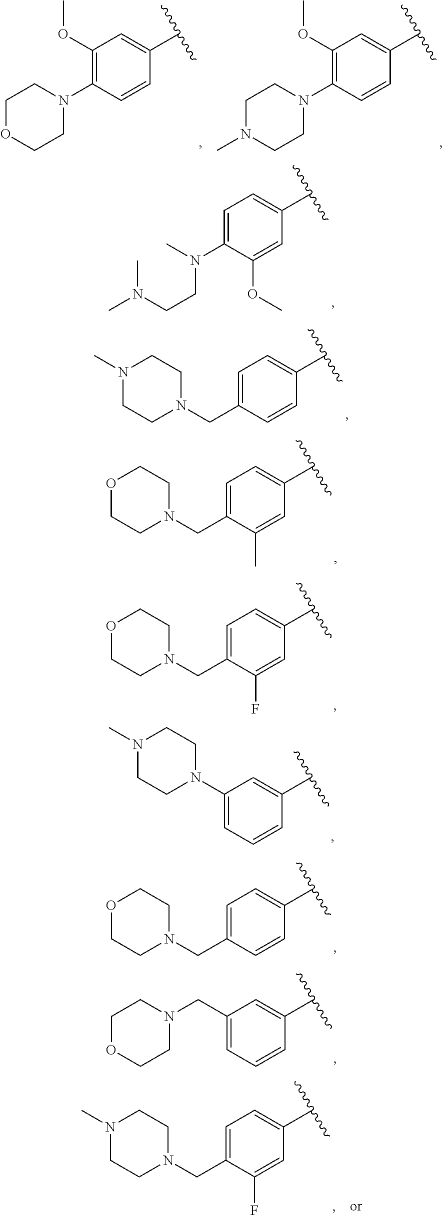

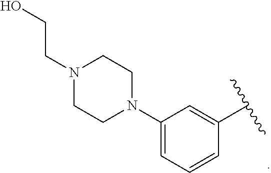 [SMILES: *c1cccc(N2CCN(CCO)CC2)c1]

Aspect 92. The compound according to aspect 81, wherein Ar is

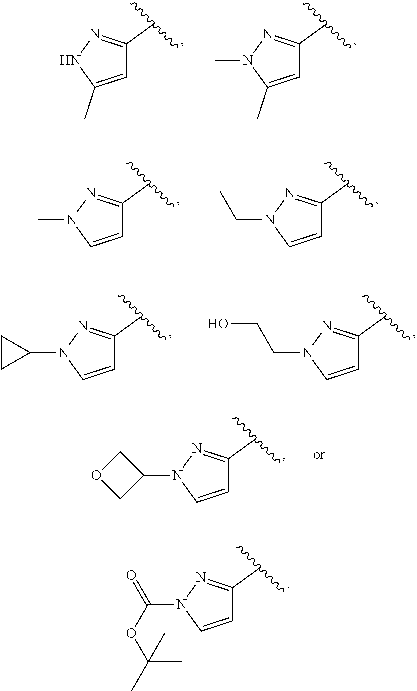

Aspect 93. The compound according to aspect 81, wherein Ar is

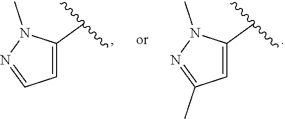

Aspect 94. The compound according to aspect 81, wherein Ar is

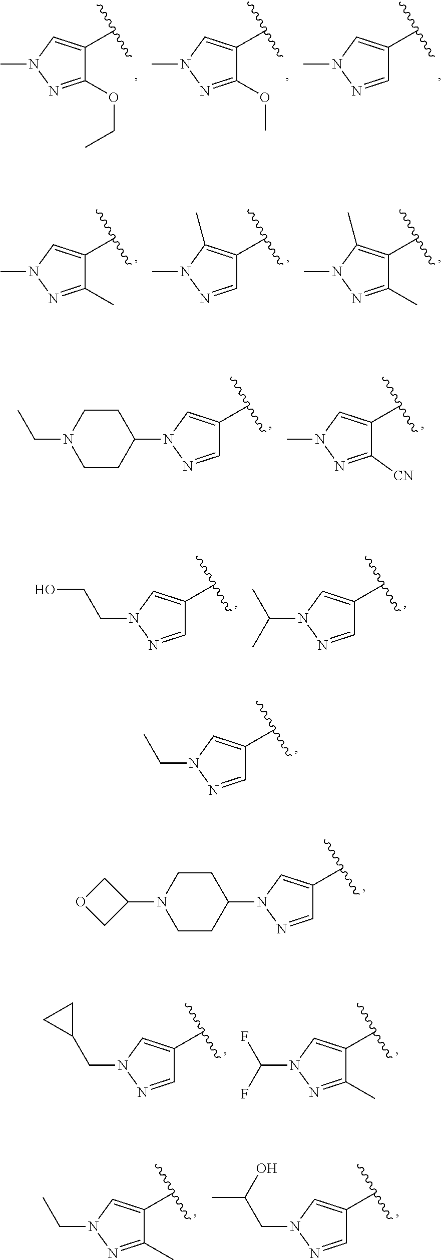

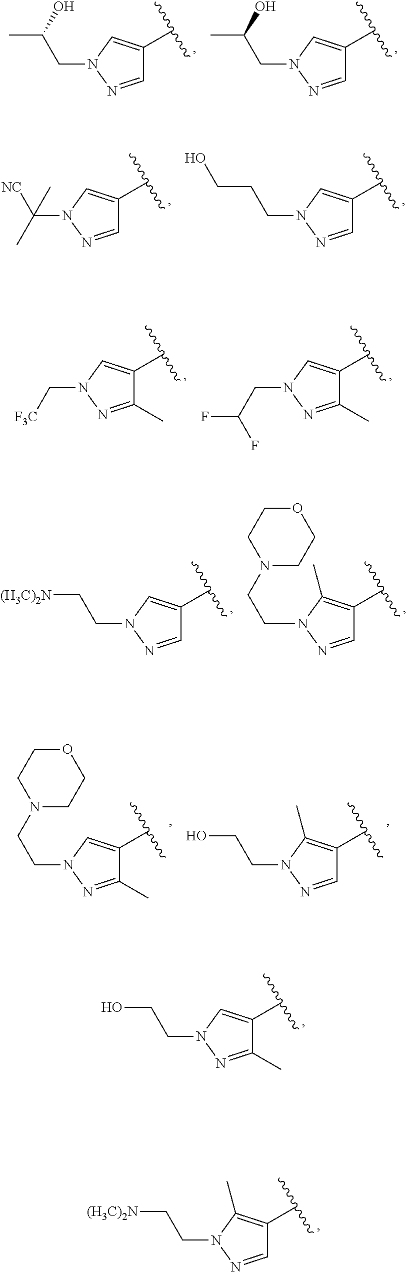

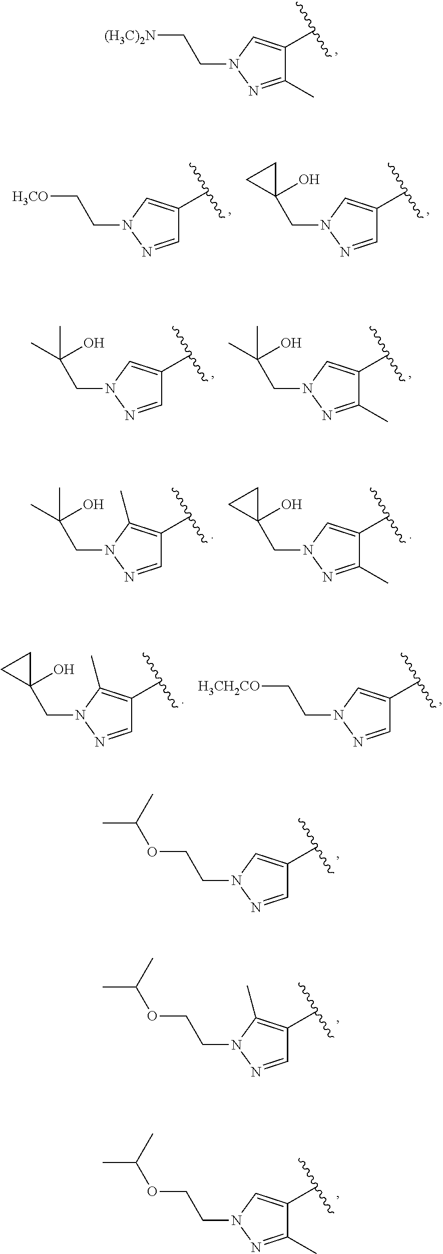

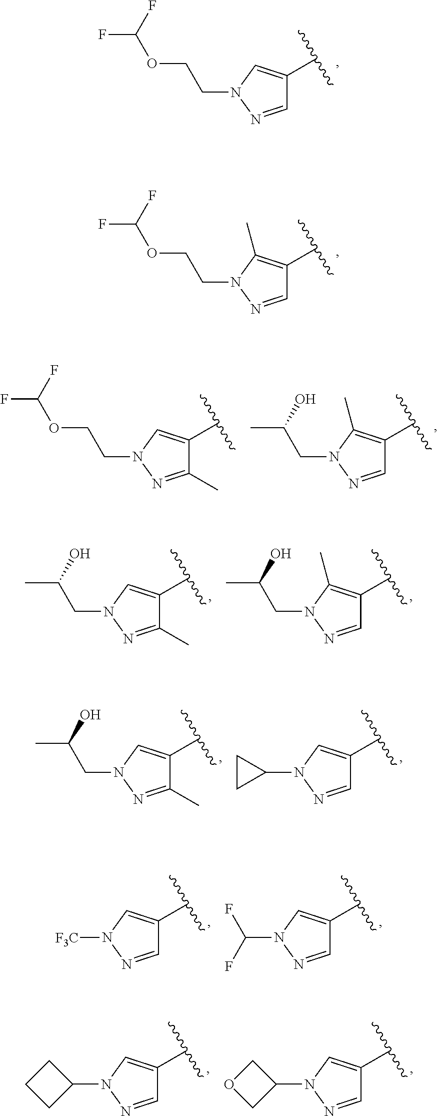

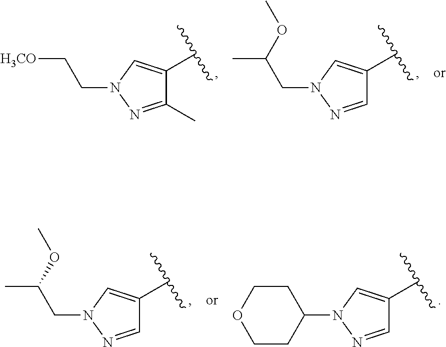

Aspect 95. The compound according to aspect 81, wherein Ar is

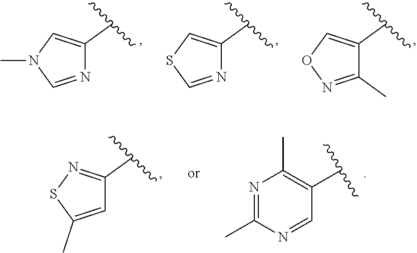

Aspect 96. The compound according to aspect 81, wherein Ar is

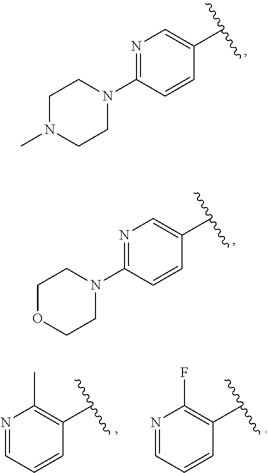

Aspect 97. The compound according to aspect 81, wherein Ar is

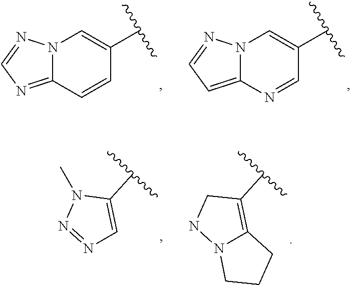

Aspect 98. The compound according to aspect 66, wherein the compound of formula IA is a compound of formula IA-1.

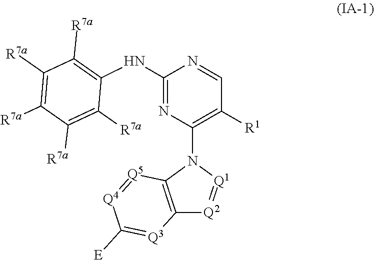

wherein each R^{7a} is independently H, —CH₃, —CH₂CH₃, isopropyl, —CH₂CF₃, —CHF₂, —CF₃, —CH₂-cyclopropyl, —CH₂CH₂OH,

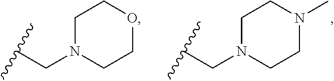

cyclopropyl, cyclobutyl,

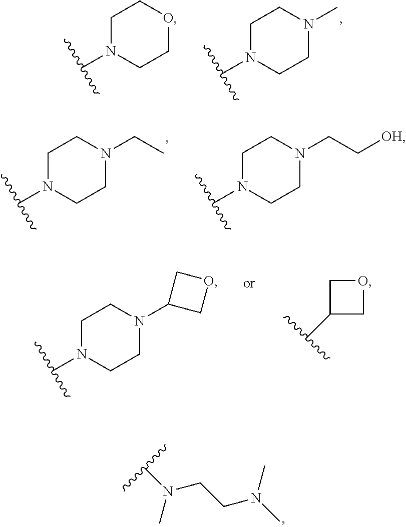

—OCH₃, —OCH₂CH₃,

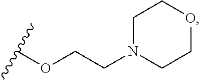 [SMILES: *OCCN1CCOCC1]

### —CN or —F.

Aspect 99. The compound according to aspect 66, wherein the compound of formula IA is a compound of formula IA-2:

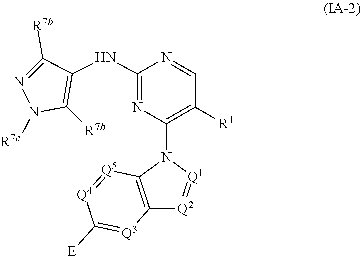

wherein each R^{7b} is independently H, —CH₃, —CH₂CH₃, isopropyl, CH₂CF₃, —CHF₂, —CF₃, —CH₂-cyclopropyl, —CH₂CH₂OH,

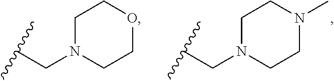

cyclopropyl, cyclobutyl,

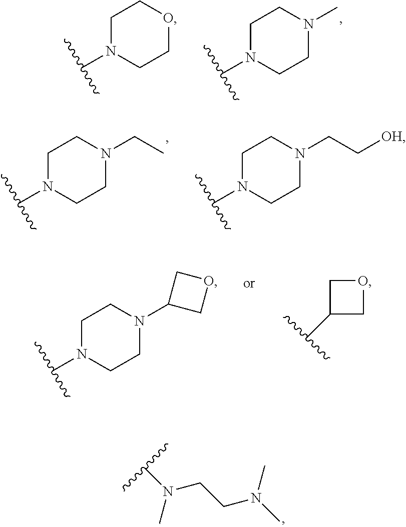

—OCH₃, —OCH₂CH₃,

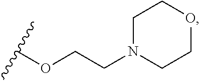 [SMILES: *OCCN1CCOCC1]

### —CN, or —F;

and R^{7c} is H, —CH₃, —CH₂CH₃, isopropyl, —CH₂CF₃, —CHF₂, —CF₃, —CH₂-cyclopropyl, —CH₂CH₂OH, is —CH₂CHF₂; —CH₂—CH(OH)—CH₃, —CH₂C(OH)(CH₃)₂, —CH₂CH₂CH₂OH, —C(CH₃)₂—CN, —CH₂CH₂OCH₃, —CH₂CH₂OCH₂CH₃, —CH₂CH₂OCH(CH₃)₂, —CH₂CH₂OCHF₂, —CH₂CH₂N(CH₃)₂,

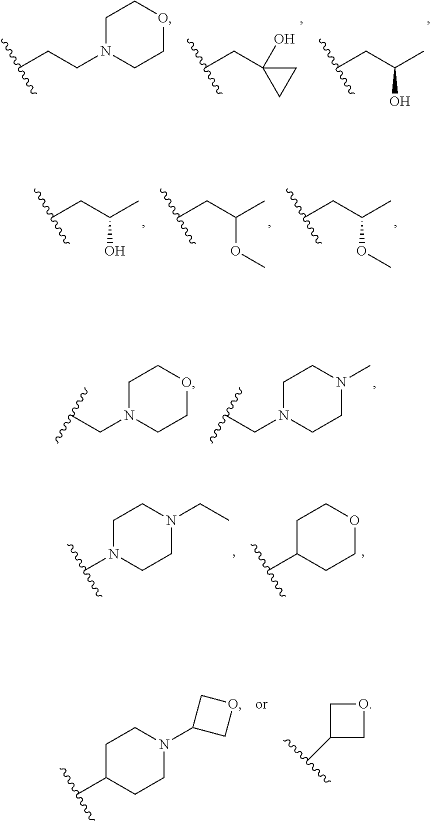

cyclopropyl, cyclobutyl,

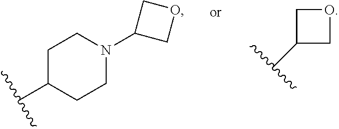 [SMILES: *C1CCN(C2COC2)CC1 ; *C1COC1]

Aspect 100. The compound according to aspect 66, wherein the compound of formula IA is a compound of formula IA-3:

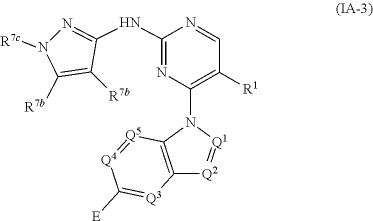

wherein each R^{7b} is independently H—CH₃, —CH₂CH₃, isopropyl, —CH₂CF₃, —CHF₂, —CF₃, —CH₂-cyclopropyl, —CH₂CH₂OH,

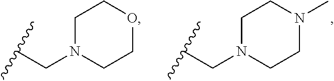

cyclopropyl, cyclobutyl,

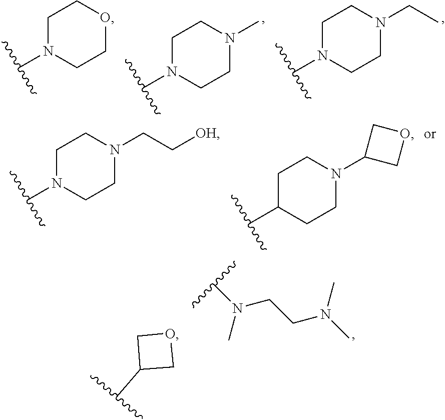

—OCH₃, —OCH₂CH₃,

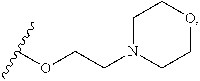 [SMILES: *OCCN1CCOCC1]

### —CN, or —F;

and R^{7c}, is H, —CH₃, —CH₂CH₃, isopropyl, —CH₂CF₃, —CHF₂, —CF₃, —CH₂-cyclopropyl, —CH₂CH₂OH,

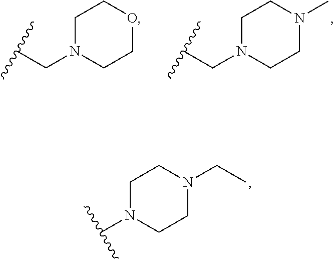

cyclopropyl, cyclobutyl, —C(O)OC(CH₃)₃,

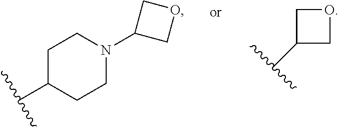 [SMILES: *C1CCN(C2COC2)CC1 ; *C1COC1]

Aspect 101. The compound according to aspect 66, wherein the compound of formula IA is a compound of formula IA-4:

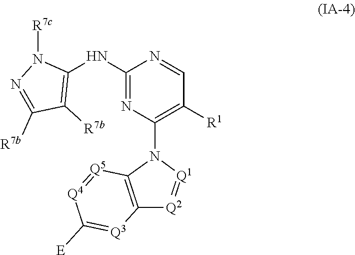

wherein each R^{7b} is independently H, —CH₃, —CH₂CH₃, isopropyl, —CH₂CF₃, —CHF₂, —CF₃, —CH₂-cyclopropyl, —CH₂CH₂OH,

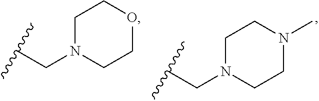

cyclopropyl, cyclobutyl,

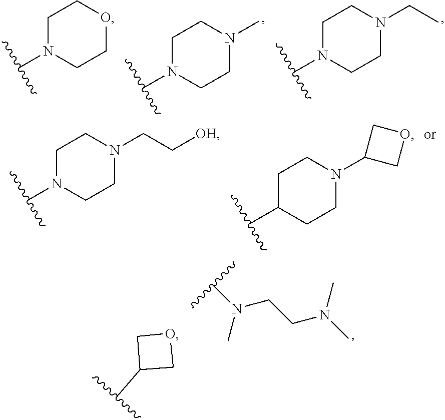

—OCH₃, —OCH₂CH₃,

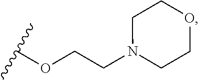 [SMILES: *OCCN1CCOCC1]

### —CN, or —F;

and R^{7c} is H, —CH₃, —CH₂CH₃, isopropyl, —CH₂CF₃, —CHF₂, —CF₃, —CH₂-cyclopropyl, —CH₂CH₂OH,

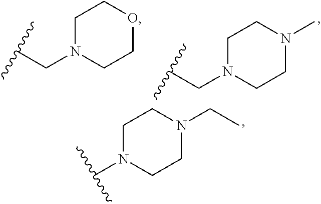

cyclopropyl, cyclobutyl,

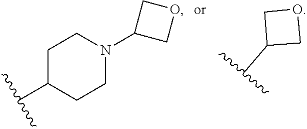 [SMILES: *C1CCN(C2COC2)CC1 ; *C1COC1]

Aspect 102. The compound according to aspect 66, wherein the compound of formula IA is a compound of formula IA-5:

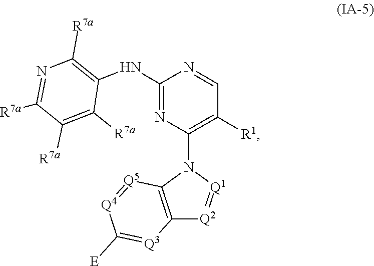

wherein each R^{7a} is independently H, —CH₃, —CH₂CH₃, isopropyl, —CH₂CF₃, —CHF₂, —CF₃, —CH₂-cyclopropyl, —CH₂CH₂OH,

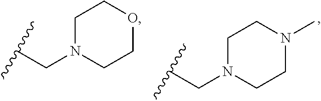

cyclopropyl, cyclobutyl,

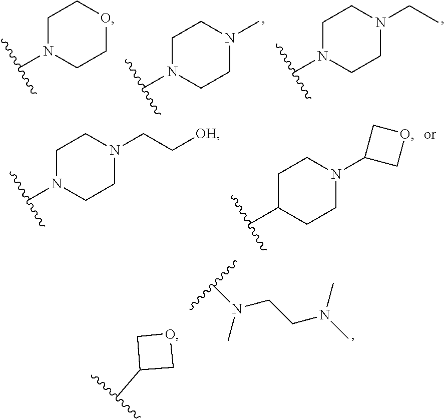

—OCH₃, —OCH₂CH₃,

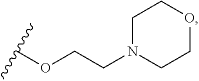 [SMILES: *OCCN1CCOCC1]

### —CN, or —F.

Aspect 103. The compound according to aspect 66, wherein the compound of formula IA is a compound of formula IA-6:

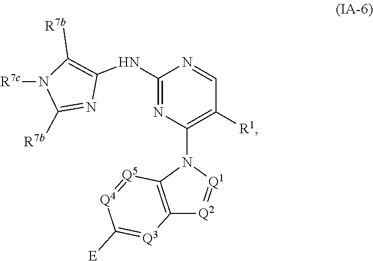

wherein each R^{7b} is independently H, —CH₃, —CH₂CH₃, isopropyl, —CH₂CF₃, —CHF₂, —CF₃, —CH₂-cyclopropyl, —CH₂CH₂OH,

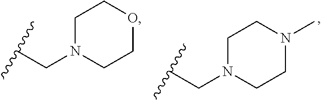

cyclopropyl, cyclobutyl,

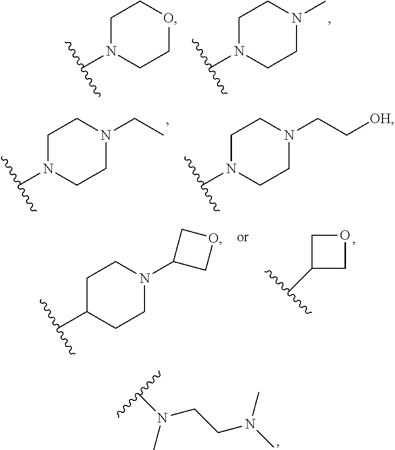

—OCH₃, —OCH₂CH₃,

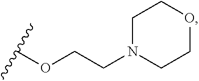 [SMILES: *OCCN1CCOCC1]

### —CN, or —F;

and R^{7c} is H, —CH₃, —CH₂CH₃, isopropyl, —CH₂CF₃, —CHF₂, —CF₃, —CH₂-cyclopropyl, —CH₂CH₂OH,

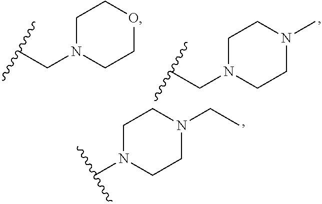

cyclopropyl, cyclobutyl,

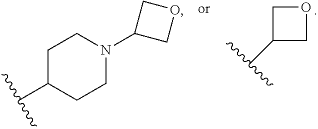 [SMILES: *C1CCN(C2COC2)CC1 ; "or" ; *C1COC1 ; "."]

Aspect 104. The compound according to any one of aspects 98-103, wherein Q¹ is C—H; Q² is C—R^{3A}; Q³ and Q⁴ are each C—R^{3B}; Q⁵ is N or C—R^{3B}; R^{3A} is H or —CH₃; and each R^{3B} is independently H or —CH₃.

Aspect 105. The compound according to any one of aspects 98-103, wherein Q¹ is C—H; Q² is C—R^{3A}; Q³ and Q⁴ are each C—R^{3B}; Q⁵ is C—R^{3B}; R^{3A}—CH₃; and each R^{3B} is H.

Aspect 106. The compound according to any one of aspects 98-103, wherein Q¹ is N; Q² is C—R^{3A}; Q³ and Q⁴ are each C—R^{3B}; Q⁵ is N or C—R^{3B}; R^{3A} is H or —CH₃; and each R^{3B} is independently H or —CH₃.

Aspect 107. The compound according to aspect 69, wherein the compound of formula IB is a compound of formula IB-1:

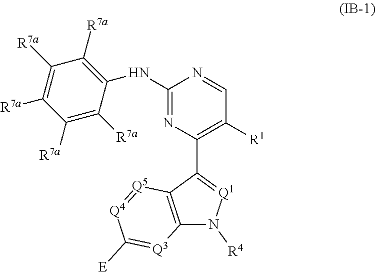

wherein each R^{7a} is independently H, —CH₃, —CH₂CH₃, isopropyl, —CH₂CF₃, —CHF₂, —CF₃, —CH₂-cyclopropyl, —CH₂CH₂OH,

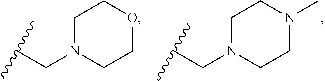

cyclopropyl, cyclobutyl,

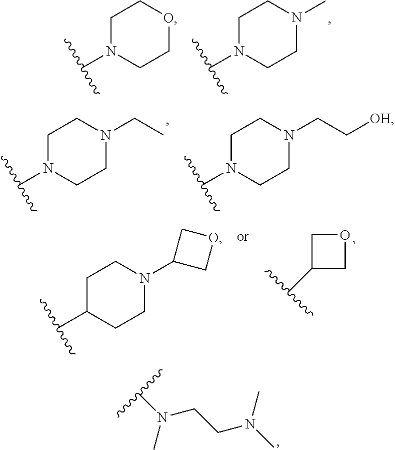

—OCH₃, —OCH₂CH₃,

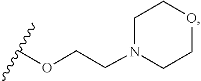 [SMILES: *OCCN1CCOCC1]

### —CN, or —F.

Aspect 108. The compound according to aspect 69, wherein the compound of formula IB is a compound of formula IB-2:

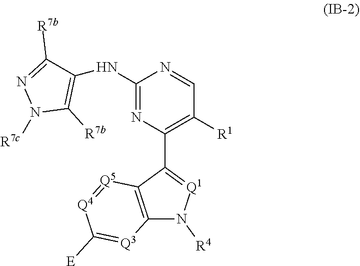

wherein each R^{7b} is independently H, —CH₃, —CH₂CH₃, isopropyl, —CH₂CF₃, —CHF₂, —CF₃, —CH₂-cyclopropyl, —CH₂CH₂OH,

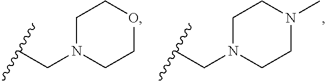

cyclopropyl, cyclobutyl,

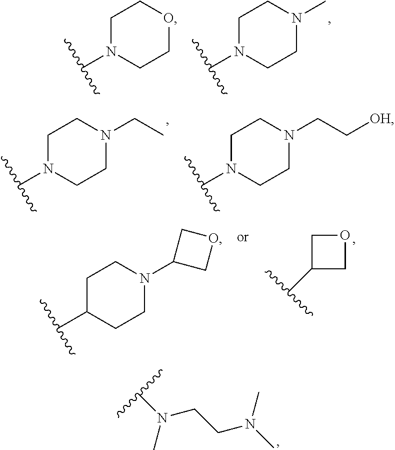

—OCH₃, —OCH₂CH₃,

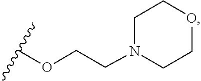 [SMILES: *OCCN1CCOCC1]

### or —F;

and R^{7c} is H, —CH₃, —CH₂CH₃, isopropyl, —CH₂CF₃, —CHF₂, —CF₃, —CH₂-cyclopropyl, —CH₂CH₂OH,

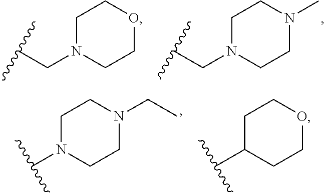

cyclopropyl, cyclobutyl, —CH₂CHF₂,

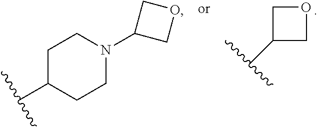 [SMILES: *C1CCN(C2COC2)CC1 ; "or" ; *C1COC1 ; "."]

Aspect 109. The compound according to aspect 69, wherein the compound of formula IB is a compound of formula IB-3:

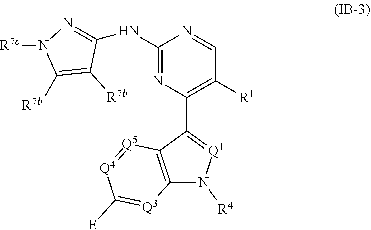

wherein each R^{7b} is independently H, —CH₃, —CH₂CH₃, isopropyl, —CH₂CF₃, —CHF₂, —CF₃, —CH₂-cyclopropyl, —CH₂CH₂OH,

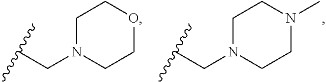

cyclopropyl, cyclobutyl,

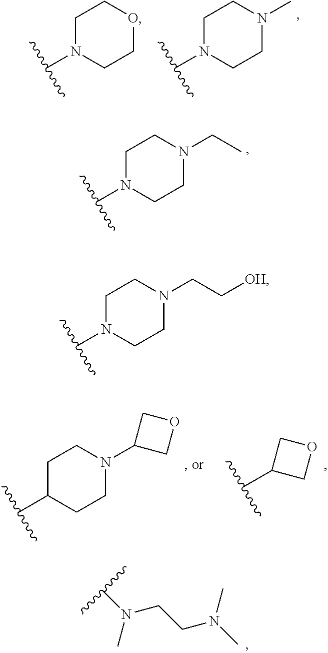

—OCH₃, —OCH₂CH₃,

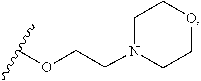 [SMILES: *OCCN1CCOCC1]

### —CN, or —F;

and R^{7c} is H—CH₃, —CH₂CH₃, isopropyl, —CH₂CF₃, —CHF₂, —CF₃, —CH₂-cyclopropyl, —CH₂CH₂OH,

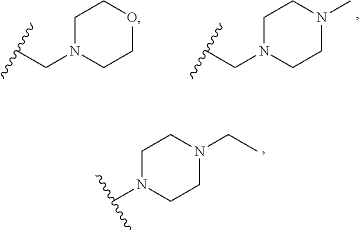

cyclopropyl, cyclobutyl,

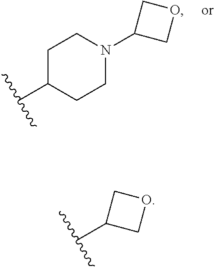 [SMILES: *C1CCN(C2COC2)CC1 ; *C1COC1]

Aspect 110. The compound according to aspect 69, wherein the compound of formula IB is a compound of formula IB-4:

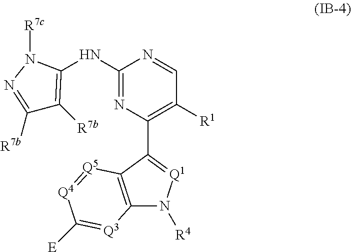

wherein each R^{7b} is independently H, —CH₃—CH₂CH₃, isopropyl, —CH₂CF₃, —CHF₂, —CF₃, —CH₂-cyclopropyl, —CH₂CH₂OH,

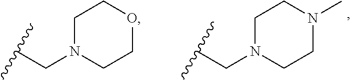

cyclopropyl, cyclobutyl,

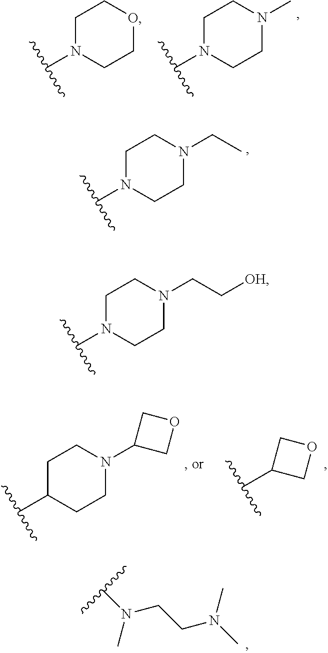

—OCH₃, —OCH₂CH₃,

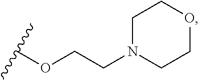 [SMILES: *OCCN1CCOCC1]

### —CN, or —F;

and R^{7c} is H, —CH₃, —CH₂CH₃, isopropyl, —CH₂CF₃, —CHF₂, —CF₃, —CH₂-cyclopropyl, —CH₂CH₂OH,

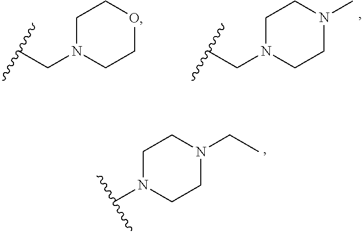

cyclopropyl, cyclobutyl,

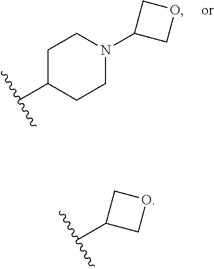 [SMILES: *C1CCN(C2COC2)CC1 ; *C1COC1]

Aspect 111. The compound according to aspect 69, wherein the compound of formula IB is a compound of formula IB-5:

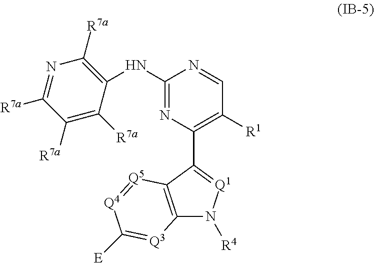

wherein each R^{7a} is independently H, —CH₃, —CH₂CH₃, isopropyl, —CH₂CF₃, —CHF₂, —CF₃, —CH₂-cyclopropyl, —CH₂CH₂OH,

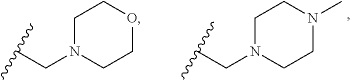

cyclopropyl, cyclobutyl,

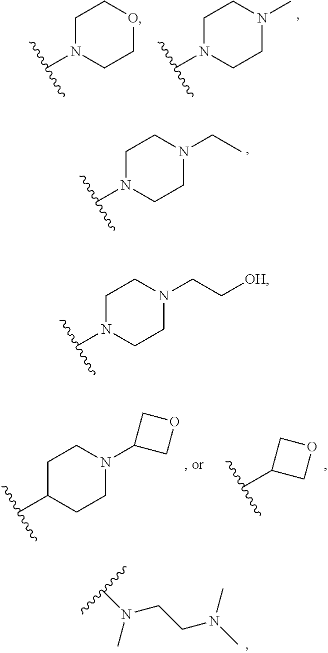

—OCH₃, —OCH₂CH₃,

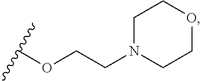 [SMILES: *OCCN1CCOCC1]

### —CN, or —F.

Aspect 112. The compound according to aspect 69, wherein the compound of formula IB is a compound of formula IB-6:

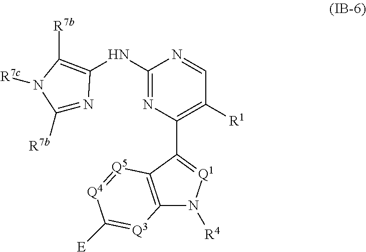

wherein each R^{7b} is independently H, —CH₃, —CH₂CH₃, isopropyl, —CH₂CF₃, —CHF₂, —CF₃, —CH₂-cyclopropyl, —CH₂CH₂OH,

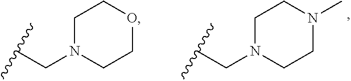

cyclopropyl, cyclobutyl,

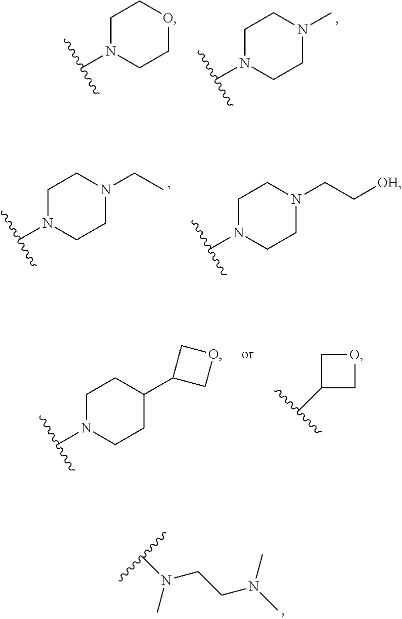

—OCH₃, —OCH₂CH₃,

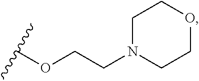 [SMILES: *OCCN1CCOCC1]

### —CN, or —F;

and R^{7c} is H, —CH₃, —CH₂CH₃, isopropyl, —CH₂CF₃, —CHF₂, —CF₃, —CH₂-cyclopropyl, —CH₂CH₂OH,

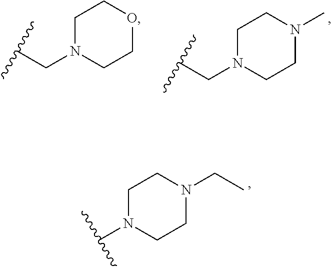

cyclopropyl, cyclobutyl,

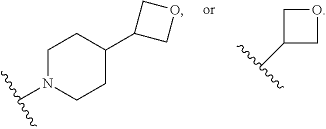 [SMILES: *N1CCC(C2COC2)CC1 ; "or" ; *C1COC1]

Aspect 113. The compound according to any one of aspects 107-112, wherein Q¹ is C—H; R⁴ is —CH₃; Q³, Q⁴, and Q⁵ are each independently N or C—R^{3B}; and each R^{3B} is independently H or —CH₃.

Aspect 114. The compound according to any one of aspects 107-112, wherein Q¹ is N; R⁴ is —CH₃, each Q³, Q⁴, and Q⁵ is N or C—R^{3B}; and each R^{3B} is independently H or —CH₃.

Aspect 115. The compound according to any one of the preceding aspects, wherein E is

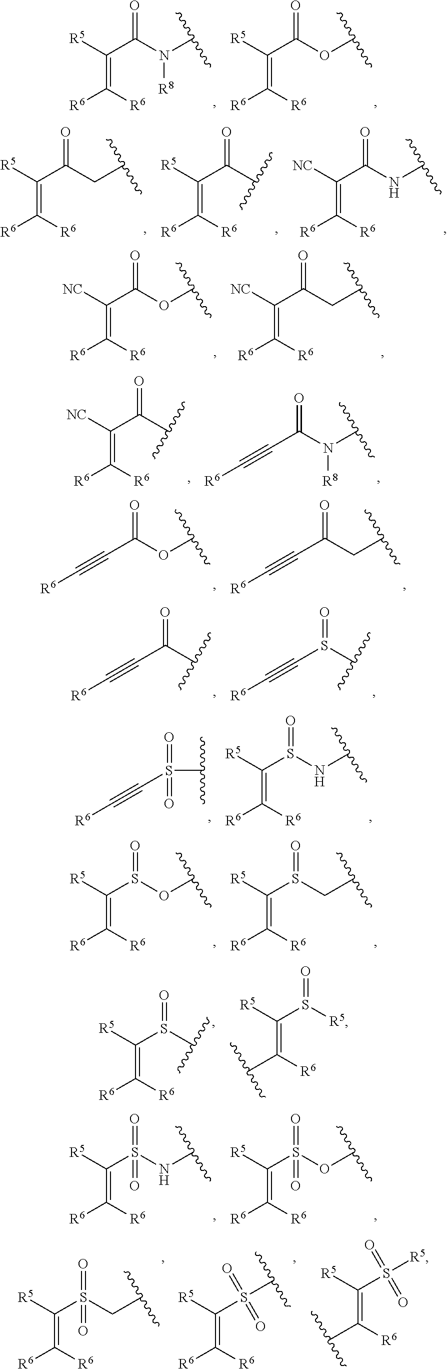

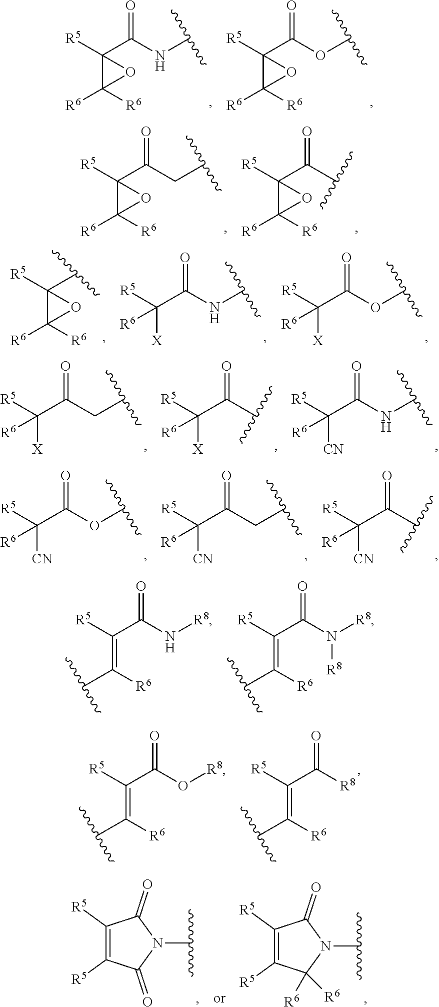

wherein each R^{c} and each is independently H, optionally substituted C₁-C₆alkyl, or halogen; R⁸ is H or C₁-C₆alkyl; and X is halogen.

Aspect 116. The compound according to aspect 115, wherein E is

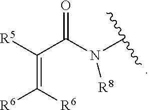

Aspect 117. The compound according to aspect 116 wherein R⁵ is H, —CH₃, or —Cl; each R⁶ is independently H or optionally substituted C₁-C₆alkyl, and each R⁸ is independently H or —CH₃.

Aspect 118. The compound according to aspect 115, wherein E is

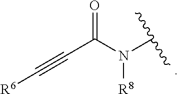

Aspect 119. The compound according to aspect 118 wherein R⁶ is H and R⁸ is H.

Aspect 120. The compound according to aspect 118 wherein R⁶ is —CH₃ and R⁸ is H or —CH₃.

Aspect 121. A pharmaceutically acceptable salt of a compound of any one of aspects 63-120.

Aspect 122. A pharmaceutical composition comprising a compound of any one of aspects 63-120 and a pharmaceutically acceptable excipient.

Aspect 123. A method of treating cancer in a subject comprising administering to the subject a therapeutically effective amount of a compound of any one of aspects 63 to 120, or a pharmaceutically acceptable salt thereof.

Aspect 124. The method of aspect 123, wherein the cancer is urothelial carcinoma, breast carcinoma, endometrial adenocarcinoma, ovarian carcinoma, primary glioma, cholangiocarcinoma, gastric adenocarcinoma, non-small cell lung carcinoma, pancreatic exocrine carcinoma, oral, prostate, bladder, colorectal carcinoma, renal cell carcinoma, neuroendocrine carcinoma, myeloproliferative neoplasms, head and neck (squamous), melanoma, leiomyosarcoma, or a sarcoma.

Aspect 125. A compound of formula I:

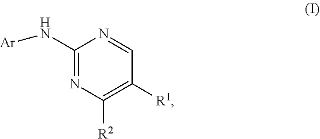

or a pharmaceutically acceptable salt thereof,
wherein:
   Ar is a 6-10-membered aryl group or a 5-10-membered heteroaryl group, each optionally substituted;
   R¹ is H, F, Cl, Br, C₁₋₆alkyl, CN, C₁₋₆haloalkyl, —C(O)NH₂, —C(O)NH(C₁-6alkyl), or —C(O)N(C₁₋₆alkyl)₂;
   R² is a:
      9-membered bicyclic heteroaryl comprising a 6-5-fused ring system wherein the 5-membered ring contains at least one nitrogen atom and the 6 membered ring is substituted with at least one E; or
      13-membered tricyclic heteroaryl comprising a 6-5-6-fused ring system wherein the 5-membered ring contains at least one nitrogen atom and one of the 6 membered rings is substituted with at least one E; or
      12- or 13-membered tricyclic group comprising a 5- or 6-membered cycloalkyl or heterocycloalkyl ring fused to a 9-membered bicyclic heteroaryl comprising a 6-5-fused ring system wherein the 5-membered ring contains at least one nitrogen atom and the 6 membered ring is substituted with at least one E;
   wherein E is an electrophilic moiety that is capable of reacting with a nucleophile so as to form a covalent bond between an atom of the nucleophile and an atom of the electrophilic moiety.

Aspect 126. The compound according to aspect 125, wherein R² is a 9-membered bicyclic heteroaryl comprising a 6-5-fused ring system wherein the 5-membered ring contains at least one nitrogen atom and the 6 membered ring is substituted with at least one E, wherein E is an electrophilic moiety that is capable of reacting with a nucleophile so as to form a covalent bond between an atom of the nucleophile and an atom of the electrophilic moiety.

Aspect 127. The compound according to aspect 125 or aspect 126, wherein R² is

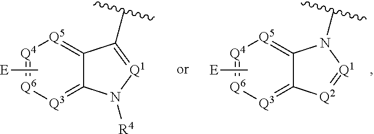

Q¹ is N or C—R^{3A};
Q² is N orC-RA,
R^{3A} is H, C₁-C₆alkyl, —CN, —C₁-C₆haloalkyl, halo, —CON(R)₂, —NR₂, cycloalkyl, —CH₂cycloalkyl, —COR, —CH(OH)R, —CO-cycloalkyl, —CH(OH)cycloalkyl, —SR, or —SO₂R; each R is independently H, or C₁-C₆alkyl;
or, when Q¹ is C—R^{3A} and Q² is C—R^{3A}, the R^{3A} of Q¹ and the R^{3A} of Q², together with the carbon atoms to which they are attached, for a 5- or 6-membered cycloalkyl or heterocycloalkyl ring, or a phenyl ring;
Q³, Q⁴, Q⁵, and Q⁶ are each independently N or C—R^{3B};
each R^{3B} is independently H, —C₁-C₆alkyl, —C₁-C₆haloalkyl, —CN, C₃-C₇cycloalkyl, or E, provided that at least one of Q³, Q⁴, Q⁵, and Q⁶ is C-E, wherein E is an electrophilic moiety that is capable of reacting with a nucleophile so as to form a covalent bond between an atom of the nucleophile and an atom of the electrophilic moiety; and
R⁴ is C₁-C₆alkyl, C₁-C₆haloalkyl, C₃-C₇cycloalkyl, or SO₂(C₁-C₆alkyl);
or, when Q¹ is C—R^{3A}, the R^{3A} of Q¹ and R⁴, together with the atoms to which they are attached, form a 5- or 6-membered heterocycloalkyl ring.

Aspect 128. The compound according to any one of aspect 125-127, wherein R² is

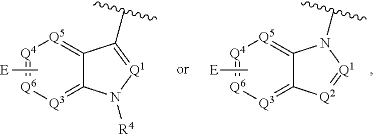

wherein
Q¹ is N or C—H;
Q² is N or C—R^{3A},
R^{3A} is H, C₁-C₆alkyl, —CN, —C₁-C₆haloalkyl, halo, —CON(R)₂, —NR₂, cycloalkyl, —CH₂cycloalkyl, —COR, —CH(OH)R, —CO-cycloalkyl, —CH(OH)cycloalkyl, —SR, or —SO₂R;
   each R is independently H, or C₁-C₆alkyl;
Q³, Q⁴, Q⁵, and Q⁶ are each independently N or C—R^{3B};
each R^{3B} is independently H, —C₁-C₆alkyl, —C₁-C₆haloalkyl, —CN, C₃-C₇cycloalkyl, or E, provided that at least one of Q³, Q⁴, Q⁵, and Q⁶ is C-E, and
R⁴ is C₁-C₆alkyl, C₁-C₆haloalkyl, C₃-C₇cycloalkyl, or SO₂(C₁-C₆alkyl).

Aspect 129. The compound according to any one of aspects 125-128, wherein R² is

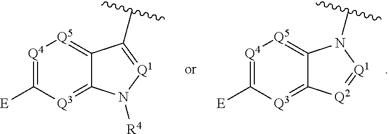

Aspect 130. The compound according to any one of aspects 125-129, wherein the compound of formula I is a compound of formula IA:

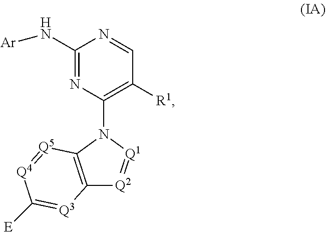

or a pharmaceutically acceptable salt thereof, wherein
Q¹ is N or C—R^{3A};
Q² is N or C—R^{3A};
   R^{3A} is H, C₁-C₆alkyl, —CN, —C₁-C₆haloalkyl, halo, —CON(R)₂, —NR₂, cycloalkyl, —CH₂cycloalkyl, —COR, —CH(OH)R, —CO-cycloalkyl, —CH(OH)cycloalkyl, —SR, or —SO₂R;
   each R is independently H, or C₁-C₆alkyl;
Q³, Q⁴, and Q⁵ are each independently N or C—R^{3B}; and
   each R^{3B} is independently H, —C₁-C₆alkyl, —C₁-C₆haloalkyl, —CN, or C₃-C₇cycloalkyl.

Aspect 131. The compound according to aspect 130, wherein Q¹ is N or C—H.

Aspect 132. The compound according to aspect 131, wherein
Q¹ is C—H;
Q² is C—R^{3A};
Q³ and Q⁴ are each C—R^{3B};
Q⁵ is N or C—R^{3B};
R^{3A} is H, —CH₃, —CH₂CH₃; and
each R^{3B} is independently H or —CH₃.

Aspect 133. The compound according to aspect 131, wherein
Q¹ is N;
Q² is C—R^{3A};
Q³ and Q⁴ are each C—R^{3B};
Q⁵ is N or C—R^{3B};
R^{3A} is H, —CH₃, —CH₂CH₃; and
each R^{3B} is independently H or —CH₃.

Aspect 134. The compound according to any one of aspects 125-129, wherein the compound of formula I is a compound of formula IB:

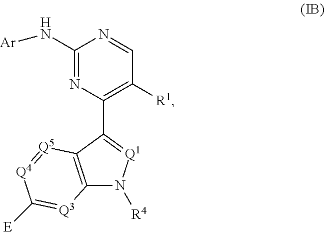

or a pharmaceutically acceptable salt thereof,
Q¹ is N or C—R^{3A};
   R^{3A} is H, C₁-C₆alkyl, —CN, —C₁-C₆haloalkyl, halo, —CON(R)₂, —NR₂, cycloalkyl, —CH₂cycloalkyl, —COR, —CH(OH)R, —CO-cycloalkyl, —CH(OH)cycloalkyl, —SR, or —SO₂R;
each R is independently H or C₁-C₆alkyl;
Q³, Q⁴, and Q⁵ are each independently N or C—R^{3B};
   each R^{3B} is independently H, —C₁-C₆alkyl, —C₁-C₆haloalkyl, —CN, or C₃-C₇cycloalkyl; and
R⁴ is C₁-C₆alkyl, C₁-C₆haloalkyl, C₃-C₇cycloalkyl, or SO₂(C₁-C₆alkyl).

Aspect 135. The compound according to aspect 134, wherein the Q¹ is N or C—H.

Aspect 136. The compound according to aspect 135, wherein
Q¹ is C—H;
R⁴ is —CH₃
Q³, Q⁴, and Q⁵ are each independently N or C—R^{3B}; and
each R^{3B} is independently H or —CH₃.

Aspect 137. The compound according to aspect 135, wherein
Q¹ is N;
R⁴ is —CH₃
Q³, Q⁴, Q⁵ is N or C—R^{3B}; and
each R^{3B} is independently H or —CH₃.

Aspect 138. The compound according to anyone of aspects 125-137, wherein R¹ is H, —C₁, —F, —CH₃, or —CN.

Aspect 139. The compound according to aspect 138, wherein R¹ is —C₁.

Aspect 140. The compound according to aspect 138, wherein R¹ is —CH₃.

Aspect 141. The compound according to aspect 138, wherein R¹ is —H.

Aspect 142. The compound according to aspect 138, wherein R¹ is —F.

Aspect 143. The compound according to aspect 138, wherein R¹ is —CN.

Aspect 144. The compound according to any one of aspects 125-143, wherein Ar is an optionally substituted 6-10-membered aryl group.

Aspect 145. The compound according to aspect 144, wherein the optionally substituted 6-10-membered aryl group is an optionally substituted phenyl group.

Aspect 146. The compound according to aspect 145, wherein the phenyl group is substituted with one or more optionally substituted C₁-C₆alkyl, optionally substituted C₃-C₆cycloalkyl, optionally substituted 4-6-membered heterocycloalkyl, —N(C₁-C₆alkyl)(optionally substituted C₁-C₆alkyl), —O(optionally substituted C₁-C₆alkyl), —CN, or halogen.

Aspect 147. The compound according to any one of aspects 125 to 143, wherein Ar is an optionally substituted 5-10-membered heteroaryl group.

Aspect 148. The compound according to aspect 147, wherein the 5-10-membered heteroaryl group is a pyridinyl, a pyrazolyl, a triazolyl, an imidazolyl, a pyrazolopyrimidine, or a triazolopyridine.

Aspect 149. The compound according to any one of aspects 147 or 148, wherein the 5-10-membered heteroaryl group is substituted with one or more optionally substituted C₁-C₆alkyl, optionally substituted C₃-C₆cycloalkyl, optionally substituted 4-6-membered heterocycloalkyl, —N(C₁-C₆alkyl)(optionally substituted C₁-C₆alkyl), —O(optionally substituted C₁-C₆alkyl), —CN, halogen, or —C(O)—O(optionally substituted C₁-C₆alkyl).

Aspect 150. The compound according to aspect 146 or aspect 149, wherein the optionally substituted C₁-C₆alkyl is —CH₃, —CH₂CH₃, isopropyl, —CH₂CF₃, —CHF₂, —CF₃, —CH₂-cyclopropyl, —CH₂-(optionally substituted 5 or 6-membered heterocycloalkyl), —CH₂CH₂OH, —CH₂CH(OH)CH₃, —CH₂C(OH)(CH₃)₂, —C(CN)(CH₃)₂, —CH₂CH₂CH₂OH, —CH₂CH₂OCH₃, —CH₂CH₂OCH₂CH₃, —CH₂CH₂OCH(CH₃)₂, CH₂CH(OCH₃)CH₃, —CH₂CHF₂, —CH₂CH₂N(CH₃)₂, —CH₂CH₂-(optionally substituted 5 or 6-membered heterocycloalkyl), or —CH₂-optionally substituted cyclopropyl.

Aspect 151. The compound according to aspect 150, wherein the —CH₂-(optionally substituted 5 or 6-membered heterocycloalkyl) is

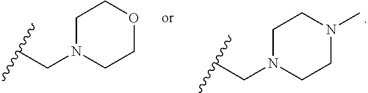

Aspect 152. The compound according to aspect 146 or aspect 149, wherein the optionally substituted C₃-C₆cycloalkyl is cyclopropyl or cyclobutyl.

Aspect 153. The compound according to aspect 146 or aspect 149, wherein the optionally substituted 4-6-membered heterocycloalkyl is

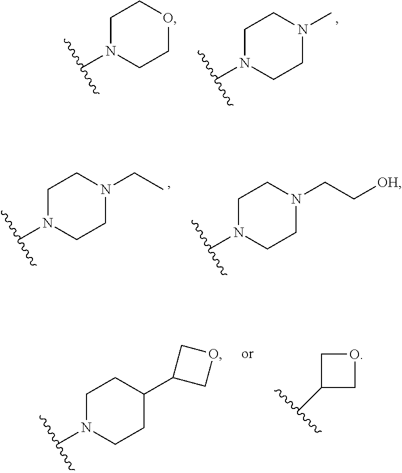

Aspect 154. The compound according to aspect 146 or aspect 149, wherein the —N(C₁-C₆alkyl)(optionally substituted C₁-C₆alkyl) is

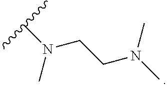 [SMILES: *N(C)CCN(C)C]

Aspect 155. The compound according to aspect 146 or aspect 149, wherein the —O(optionally substituted C₁-C₆alkyl) is —OCH₃, —OCH₂CH₃, or

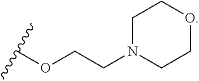 [SMILES: *OCCN1CCOCC1]

Aspect 156. The compound according to aspect 146 or aspect 149, wherein the halogen is —F.

Aspect 157. The compound according to aspect 144, wherein Ar is

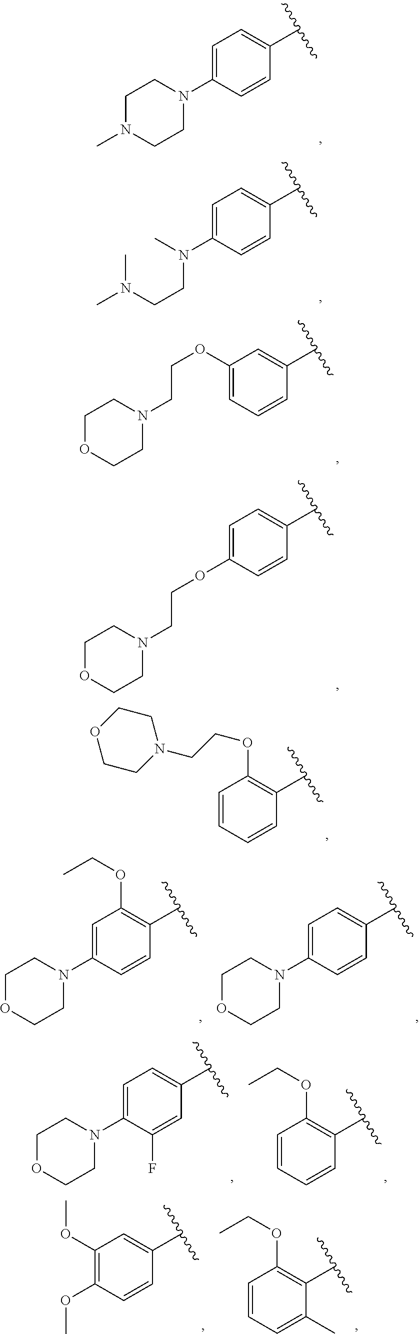

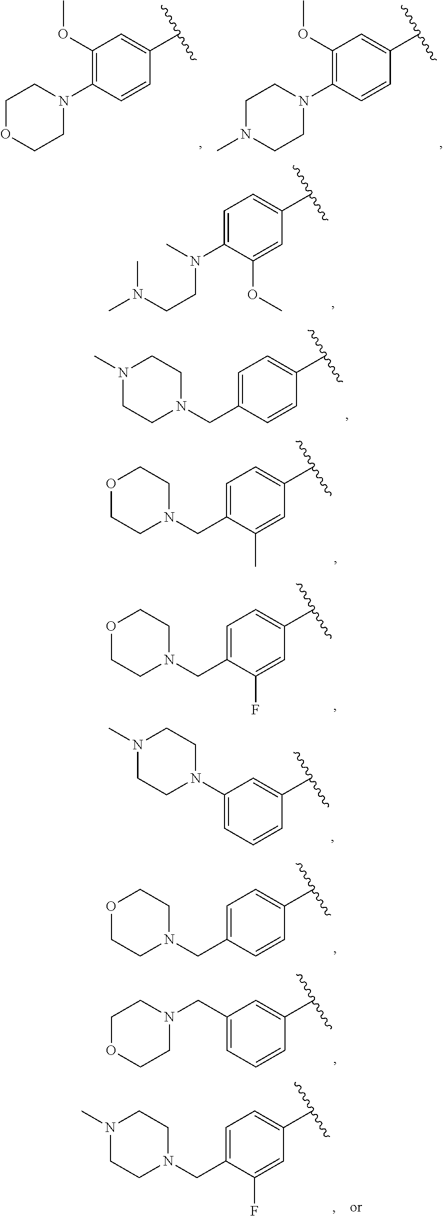

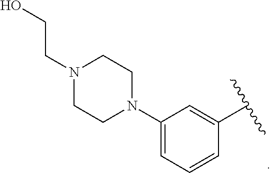 [SMILES: *c1cccc(N2CCN(CCO)CC2)c1]

Aspect 159. The compound according to aspect 147, wherein Ar is

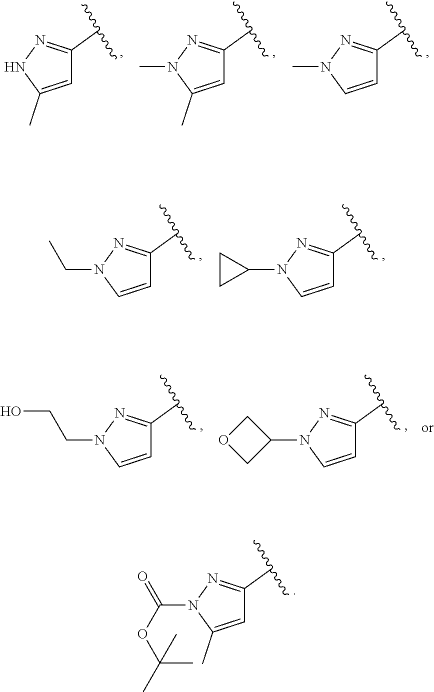

Aspect 160. The compound according to aspect 147, wherein Ar is

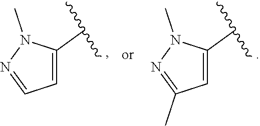

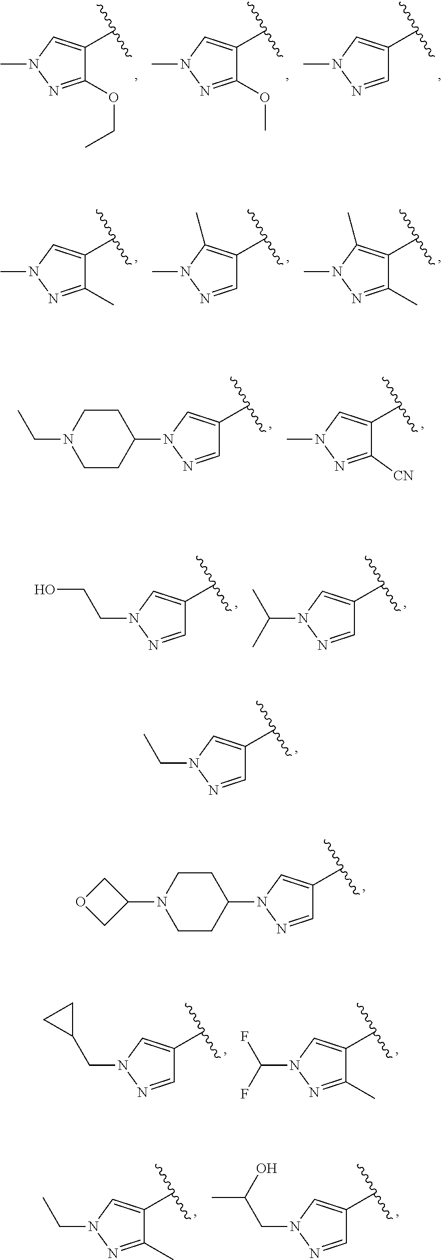

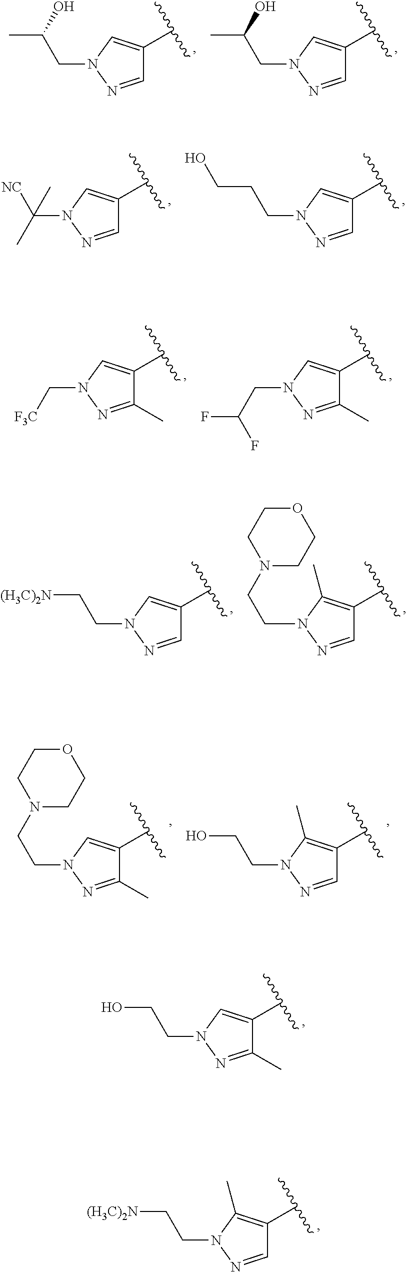

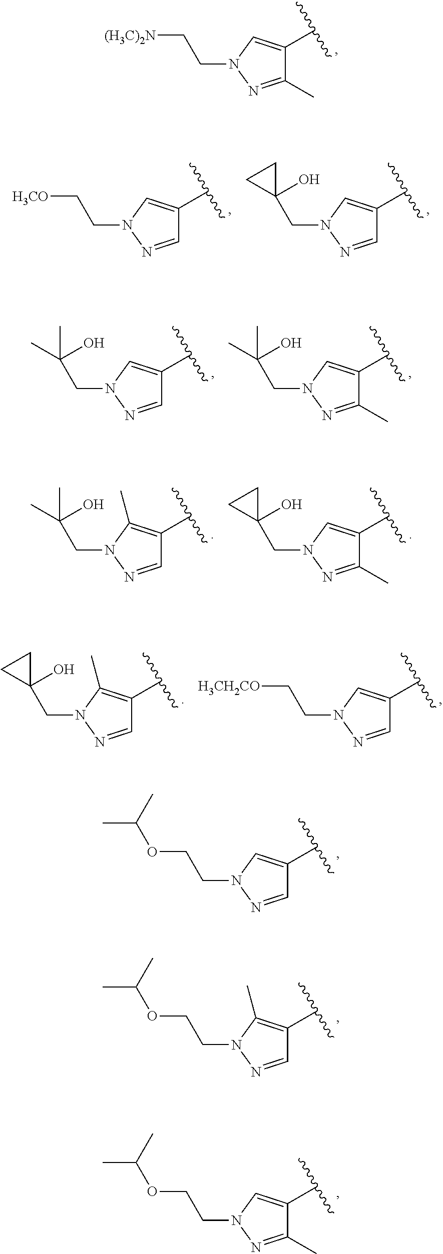

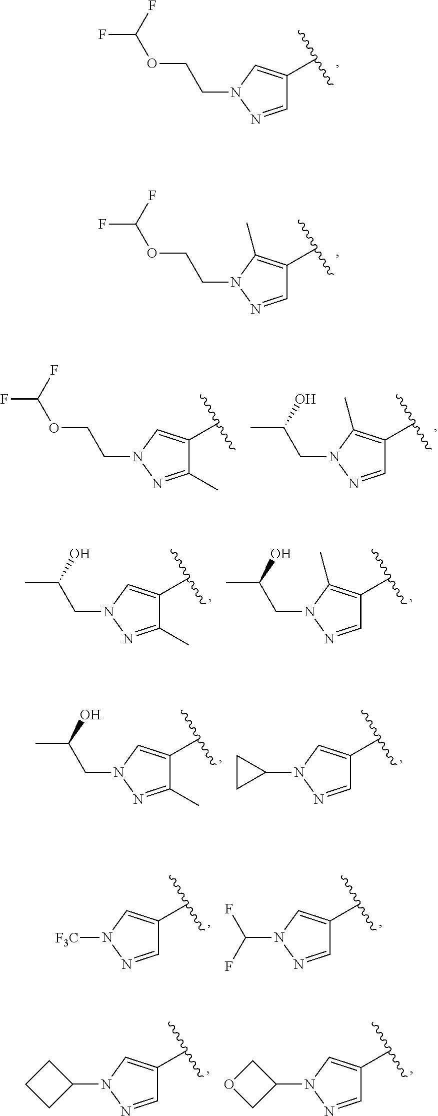

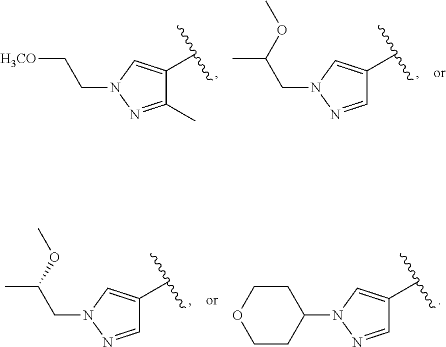

Aspect 161. The compound according to aspect 147, wherein Ar is

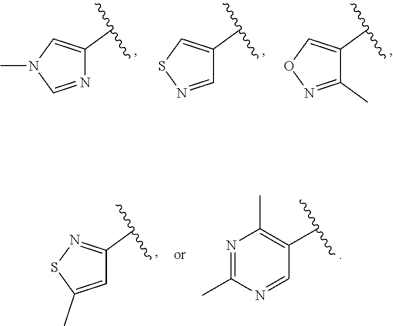

Aspect 162. The compound according to aspect 147, wherein Ar is

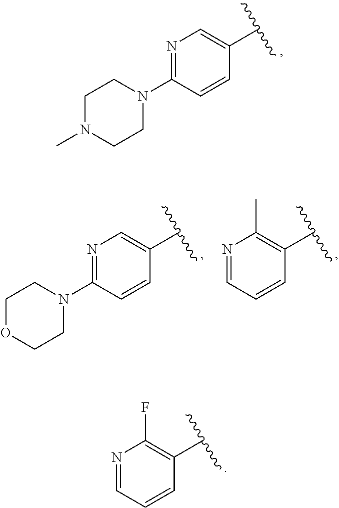

Aspect 163. The compound according to aspect 147, wherein Ar is

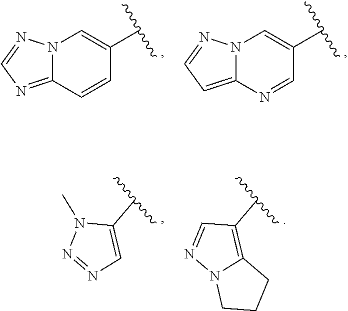

Aspect 164. The compound according to any one of aspects 125-131, wherein the compound of formula IA is a compound of formula IA-1:

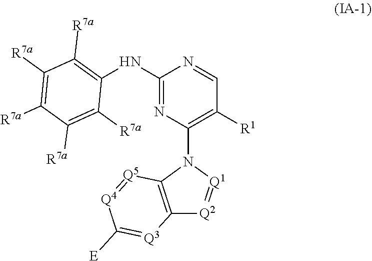

wherein each R^{7a} is independently H, —CH₃, —CH₂CH₃, isopropyl, —CH₂CF₃, —CHF₂, —CF₃, —CH₂-cyclopropyl, —CH₂CH₂OH,

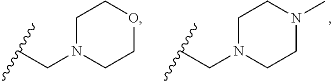

cyclopropyl, cyclobutyl,

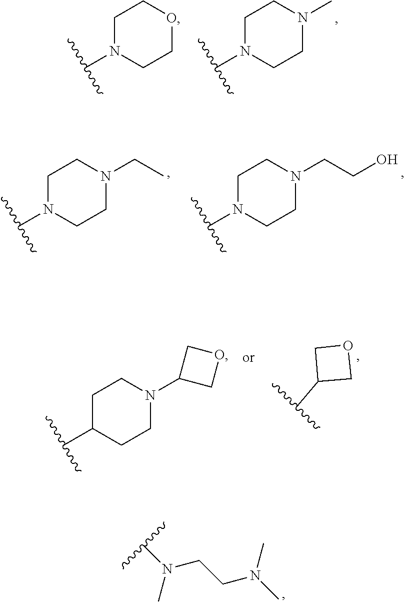

—OCH₃, —OCH₂CH₃,

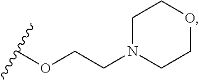 [SMILES: *OCCN1CCOCC1]

### —CN, or —F.

Aspect 165. The compound according to any one of aspects 125-131, wherein the compound of formula IA is a compound of formula IA-2:

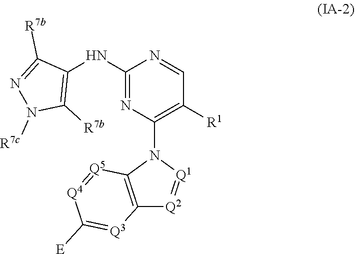

wherein each R^{7b} is independently H—CH₃—CH₂CH₃, isopropyl, —CH₂CF₃, —CHF₂, —CF₃, —CH₂-cyclopropyl, —CH₂CH₂OH,

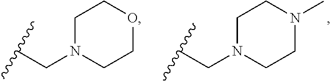

cyclopropyl, cyclobutyl,

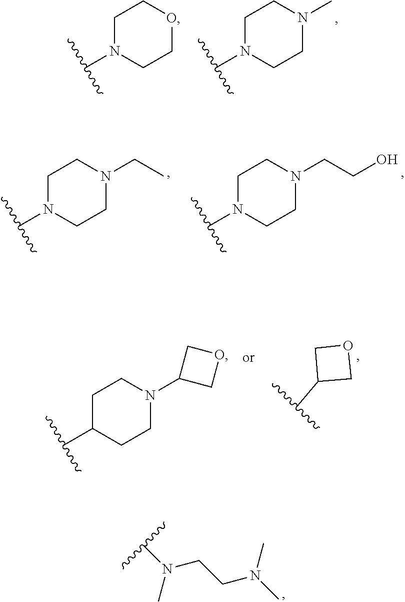

—OCH₃, —OCH₂CH₃,

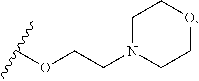 [SMILES: *OCCN1CCOCC1]

### —CN, or —F;

and R^{7c} is H, —CH₃, —CH₂CH₃, isopropyl, —CH₂CF₃, —CHF₂, —CF₃, —CH₂-cyclopropyl, —CH₂CH₂OH, is —CH₂CHF₂; —CH₂—CH(OH)—CH₃, —CH₂C(OH)(CH₃)₂, —CH₂CH₂CH₂OH, —C(CH₃)₂—CN, —CH₂CH₂OCH₃, —CH₂CH₂OCH₂CH₃, —CH₂CH₂OCH(CH₃)₂, —CH₂CH₂OCHF₂, CH₂CH₂N(CH₃)₂,

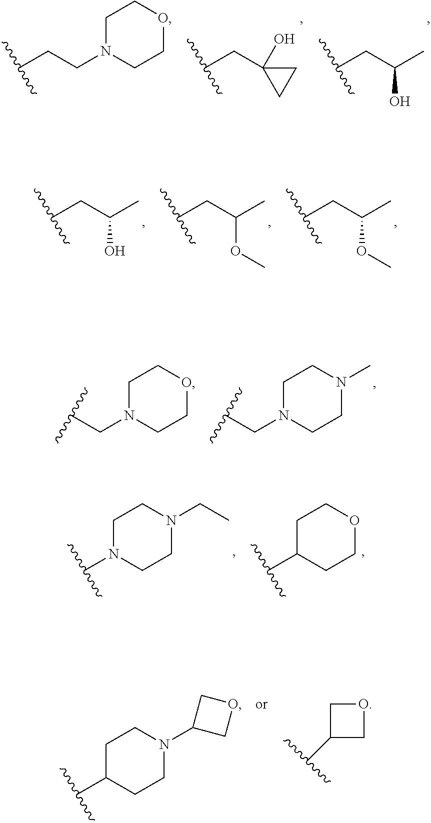

cyclopropyl, cyclobutyl,

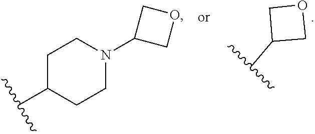 [SMILES: *C1CCN(C2COC2)CC1 ; *C1COC1]

Aspect 166. The compound according to any one of aspects 125-131, wherein the compound of formula IA is a compound of formula IA-3:

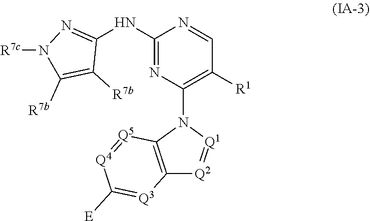

wherein each R^{7b} is independently H—CH₃, —CH₂CH₃, isopropyl, —CH₂CF₃, —CHF₂, —CF₃, —CH₂-cyclopropyl, —CH₂CH₂OH,

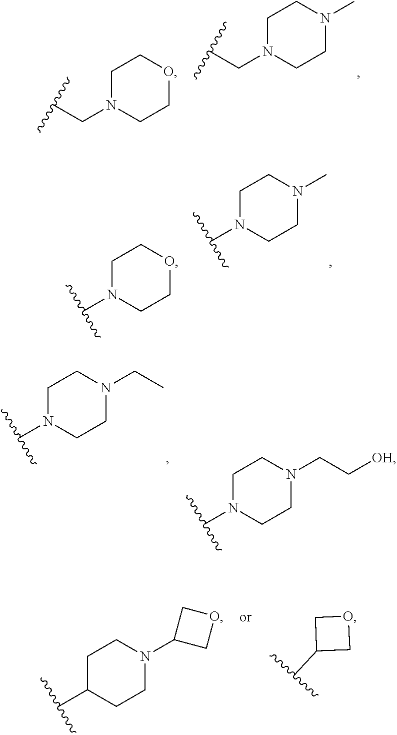

cyclopropyl, cyclobutyl,

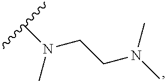 [SMILES: *N(C)CCN(C)C]

—OCH₃, —OCH₂CH₃,

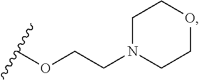 [SMILES: *OCCN1CCOCC1]

### —CN, or —F;

and R^{7c} is H, —CH₃—CH₂CH₃, isopropyl, —CH₂CF₃—CHF₂—CF₃, —CH₂-cyclopropyl, —CH₂CH₂OH,

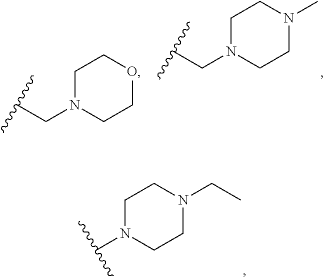

cyclopropyl, cyclobutyl, —C(O)OC(CH₃)₃,

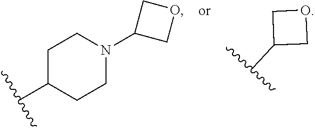 [SMILES: *C1CCN(C2COC2)CC1 ; *C1COC1]

Aspect 167. The compound according to any one of aspects 125-131, wherein the compound of formula IA is a compound of formula IA-4:

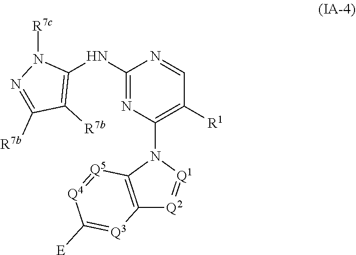

wherein each R^{7b} is independently H, —CH₃, —CH₂CH₃, isopropyl, —CH₂CF₃—CHF₂, —CF₃, —CH₂-cyclopropyl, —CH₂CH₂OH,

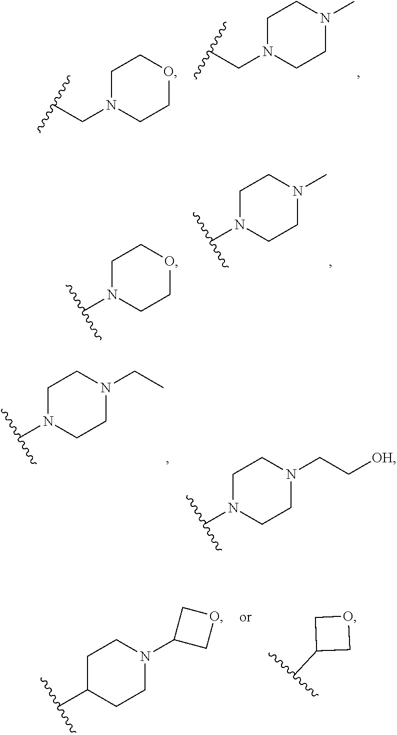

cyclopropyl, cyclobutyl,

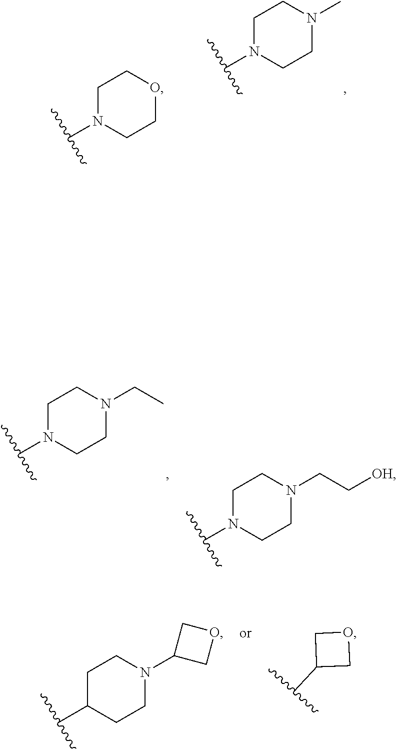

—OCH₃, —OCH₂CH₃,

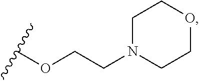 [SMILES: *OCCN1CCOCC1]

—CN, or —F; and R^{7c} is H, —CH₃, —CH₂CH₃, isopropyl, —CH₂CF₃, —CHF₂, —CF₃, —CH₂-cyclopropyl, —CH₂CH₂OH,

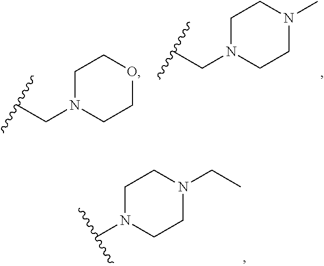

cyclopropyl, cyclobutyl,

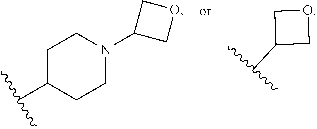 [SMILES: *C1CCN(C2COC2)CC1 ; *C1COC1]

Aspect 168. The compound according to any one of aspects 125-131, wherein the compound of formula IA is a compound of formula IA-5:

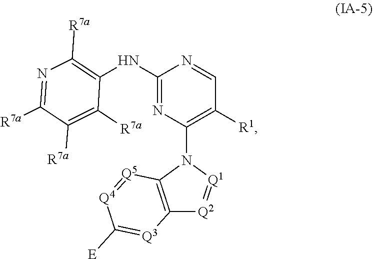

wherein each R^{7a} is independently H, —CH₃, —CH₂CH₃, isopropyl, —CH₂CF₃, —CHF₂, —CF₃, —CH₂-cyclopropyl, —CH₂CH₂OH,

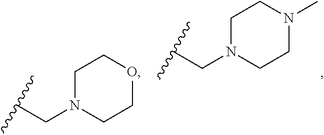

cyclopropyl, cyclobutyl,

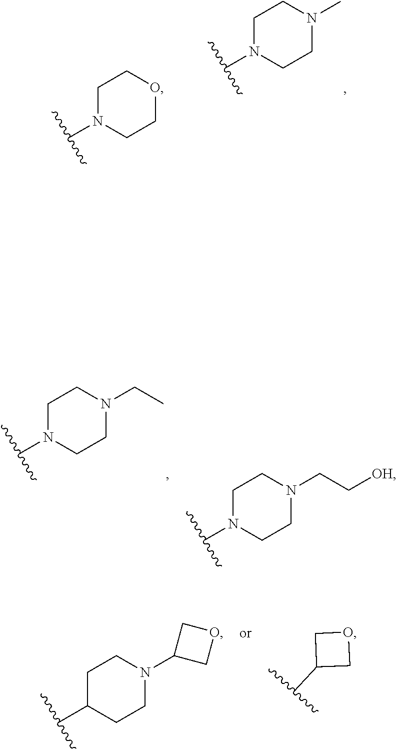

—OCH₃, —OCH₂CH₃,

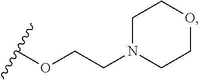 [SMILES: *OCCN1CCOCC1]

### —CN, or —F.

Aspect 169. The compound according to any one of aspects 125-131, wherein the compound of formula IA is a compound of formula IA-6:

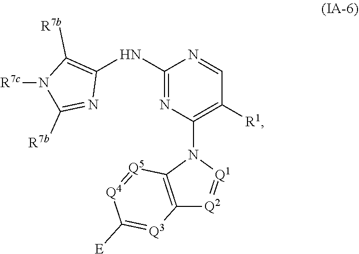

wherein each R^{7b} is independently H, —CH₃, —CH₂CH₃, isopropyl, —CH₂CF₃, —CHF₂, —CF₃, —CH₂-cyclopropyl, —CH₂CH₂OH,

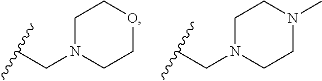

cyclopropyl, cyclobutyl,

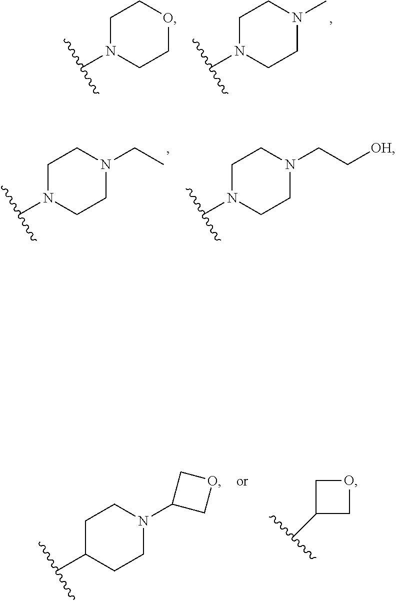

—OCH₃, —OCH₂CH₃,

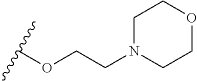 [SMILES: *OCCN1CCOCC1]

—CN, or —F; and R^{7c} is H, —CH₃, —CH₂CH₃, isopropyl, —CH₂CF₃—CHF₂—CF₃, —CH₂-cyclopropyl, —CH₂CH₂OH,

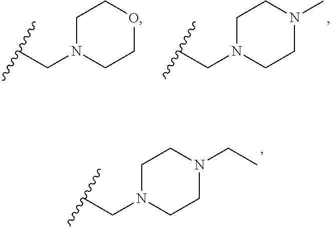

cyclopropyl, cyclobutyl,

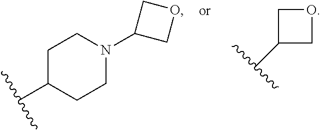 [SMILES: *C1CCN(C2COC2)CC1 ; *C1COC1]

Aspect 170. The compound according to any one of aspects 164-169, wherein Q¹ is C—H; Q² is C—R^{3A}; Q³ and Q⁴ are each C—R^{3B}; Q⁵ is N or C—R^{3B}; R^{3A} is H or —CH₃; and each R^{3B} is independently H or —CH₃.

Aspect 171. The compound according to any one of aspects 164-169, wherein Q¹ is C—H; Q² is C—R^{3A}; Q³ and Q⁴ are each C—R^{3B}; Q⁵ is C—R^{3B}; R^{3A}—CH₃; and each R^{3B} is H.

Aspect 172. The compound according to any one of aspects 164-169, wherein Q¹ is N; Q² is C—R^{3A}; Q³ and Q⁴ are each C—R^{3B}; Q⁵ is N or C—R^{3B}; R^{3A} is H or —CH₃; and each R^{3B} is independently H or —CH₃.

Aspect 173. The compound according to any one of aspects 164-169, wherein Q¹ is C—R^{3A}; Q² is C—R^{3A}; Q³ is C—R^{3B}; Q⁴ is C—R^{3B}; Q⁵ is C—R^{3B}; each R^{3A} is independently H or —CH₃; and each R^{3B} is independently H or —CH₃.

Aspect 174. The compound according to any one of aspects 164-169, Q¹ is C—R^{3A}; Q² is C—R^{3A}; Q³ is C—R^{3B}; Q⁴ is C—R^{3B}; Q⁵ is C—R^{3B}; each R^{3B} is independently H or —CH₃; and the R^{3A} of Q¹ and the R^{3A} of Q², together with the carbon atoms to which they are attached, form a 5- or 6-membered cycloalkyl or heterocycloalkyl ring, or a phenyl ring.

Aspect 175. The compound according to any one of aspects 125-129 or 134-135, wherein the compound of formula IB is a compound of formula IB-1:

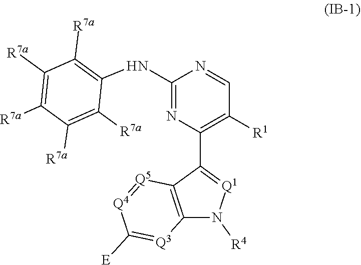

wherein each R^{7a} is independently H, —CH₃, —CH₂CH₃, isopropyl, —CH₂CF₃, —CHF₂, —CF₃, —CH₂-cyclopropyl, —CH₂CH₂OH,

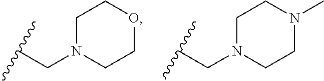

cyclopropyl, cyclobutyl,

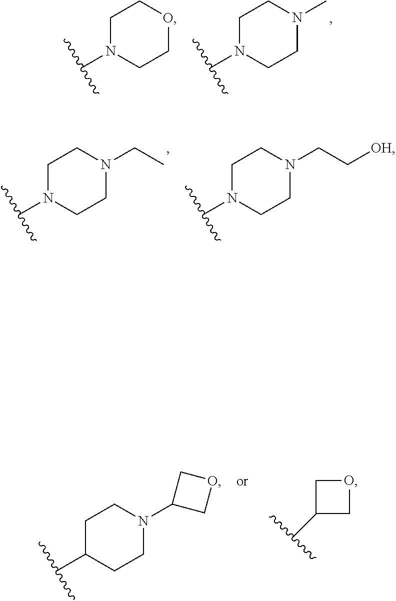

—OCH₃, —OCH₂CH₃,

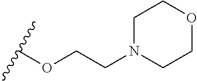 [SMILES: *OCCN1CCOCC1]

### —CN, or —F.

Aspect 176. The compound according to any one of aspects 125-129 or 134-135, wherein the compound of formula IB is a compound of formula IB-2:

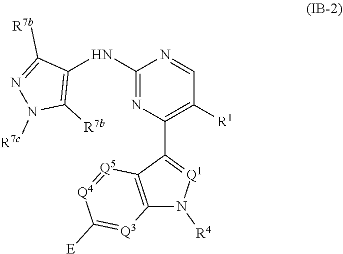

wherein each R^{7b} is independently H, —CH₃, —CH₂CH₃, isopropyl, —CH₂CF₃, —CHF₂, —CF₃, —CH₂-cyclopropyl, —CH₂CH₂OH,

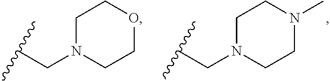

cyclopropyl, cyclobutyl,

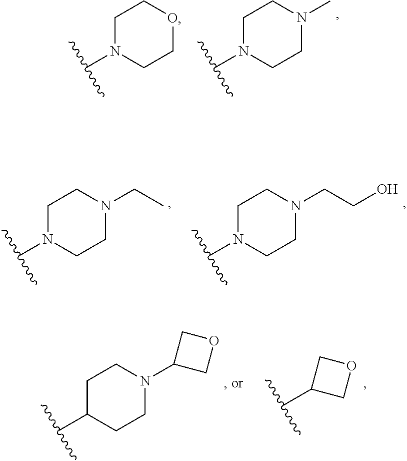

—OCH₃, —OCH₂CH₃,

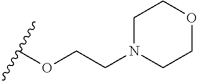 [SMILES: *OCCN1CCOCC1]

### or —F;

and R^{7c} is H, —CH₃, —CH₂CH₃, isopropyl, —CH₂CF₃, —CHF₂, —CF₃, —CH₂-cyclopropyl, —CH₂CH₂OH,

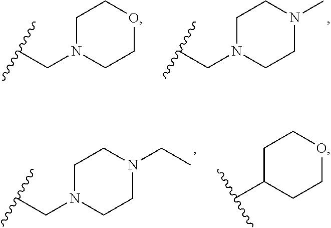

cyclopropyl, cyclobutyl, —CH₂CHF₂,

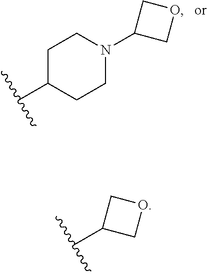 [SMILES: *C1CCN(C2COC2)CC1 ; *C1COC1]

Aspect 177. The compound according to any one of aspects 125-129 or 134-135, wherein the compound of formula IB is a compound of formula IB-3:

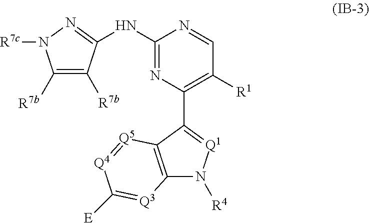

wherein each R^{7b} is independently H, —CH₃—CH₂CH₃, isopropyl, —CH₂CF₃, —CHF₂, —CF₃, —CH₂-cyclopropyl, —CH₂CH₂OH,

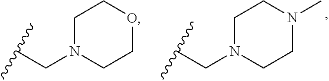

cyclopropyl, cyclobutyl,

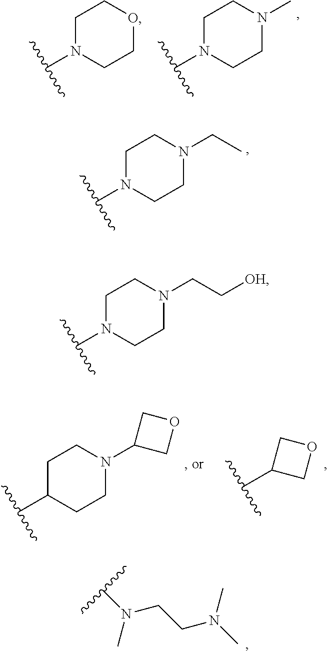

—OCH₃, —OCH₂CH₃,

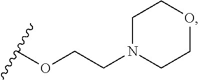 [SMILES: *OCCN1CCOCC1]

### —CN, or —F;

and R^{7c} is H—CH₃, —CH₂CH₃, isopropyl, —CH₂CF₃, —CHF₂, —CF₃, —CH₂-cyclopropyl, —CH₂CH₂OH,

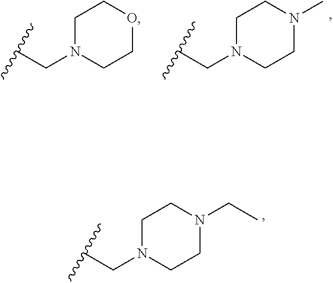

cyclopropyl, cyclobutyl,

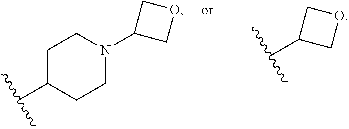 [SMILES: *C1CCN(C2COC2)CC1 ; *C1COC1]

Aspect 178. The compound according to any one of aspects 125-129 or 134-135, wherein the compound of formula IB is a compound of formula IB-4:

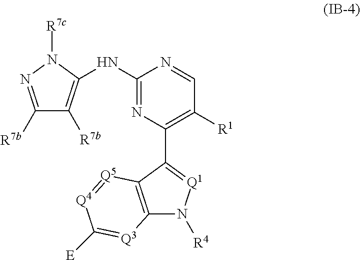

wherein each R^{7b} is independently H, —CH₃, —CH₂CH₃, isopropyl, —CH₂CF₃, —CHF₂, —CF₃, —CH₂-cyclopropyl, —CH₂CH₂OH,

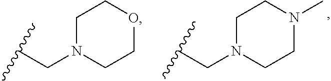

cyclopropyl, cyclobutyl,

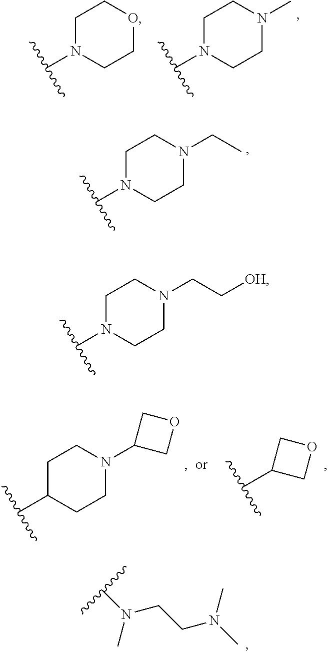

—OCH₃, —OCH₂CH₃,

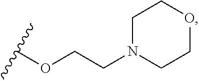 [SMILES: *OCCN1CCOCC1]

### —CN, or —F;

and R^{7c} is H, —CH₃, —CH₂CH₃, isopropyl, —CH₂CF₃, —CHF₂, —CF₃, —CH₂-cyclopropyl, —CH₂CH₂OH, cyclopropyl, cyclobutyl,

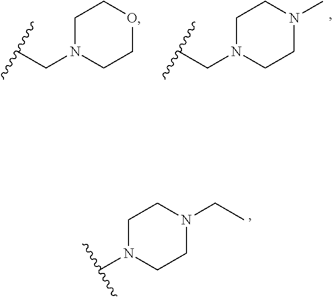

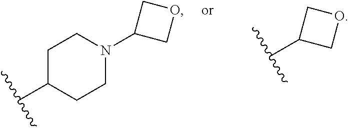 [SMILES: *C1CCN(C2COC2)CC1 ; *C1COC1]

Aspect 179. The compound according to any one of aspects 125-129 or 134-135, wherein the compound of formula IB is a compound of formula IB-5:

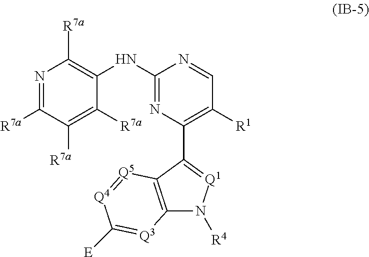

wherein each R^{7a} is independently H, —CH₃, —CH₂CH₃, isopropyl, —CH₂CF₃, —CHF₂, —CF₃, —CH₂-cyclopropyl, —CH₂CH₂OH,

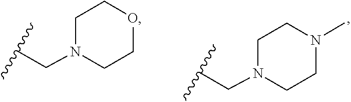

cyclopropyl, cyclobutyl,

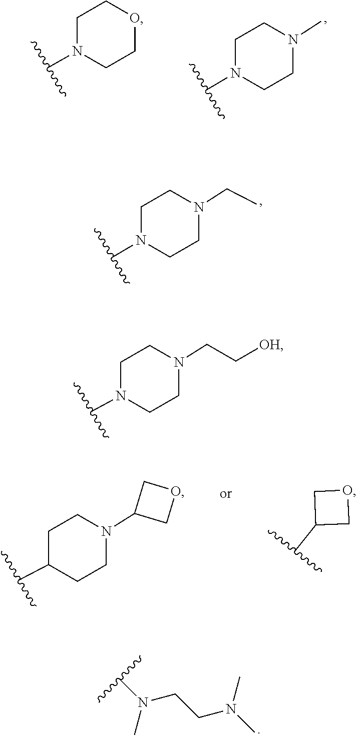

—OCH₃, —OCH₂CH₃,

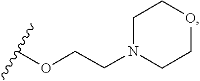 [SMILES: *OCCN1CCOCC1]

### —CN, or —F.

Aspect 180. The compound according to any one of aspects 125-129 or 134-135, wherein the compound of formula IB is a compound of formula IB-6:

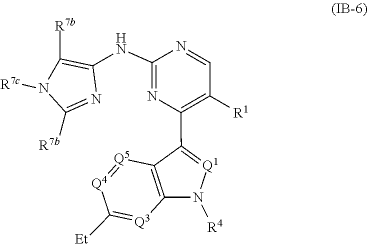

wherein each R^{7b} is independently H, —CH₃, —CH₂CH₃, isopropyl, —CH₂CF₃, —CHF₂, —CF₃, —CH₂-cyclopropyl, —CH₂CH₂OH,

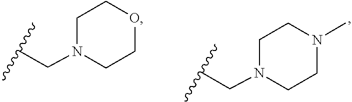

cyclopropyl, cyclobutyl,

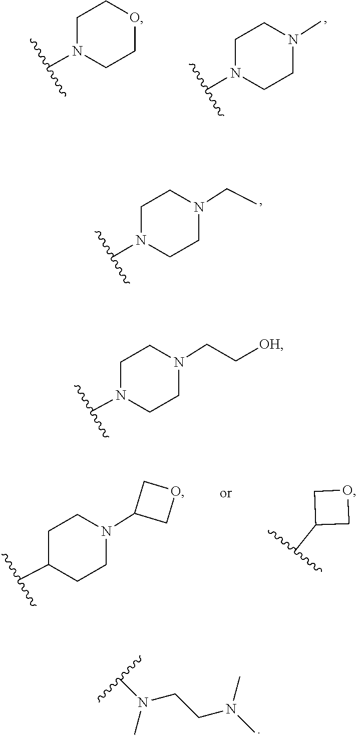

—OCH₃, —OCH₂CH₃,

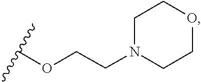 [SMILES: *OCCN1CCOCC1]

—CN, or —F; and R^{7c} is H, —CH₃, —CH₂CH₃, isopropyl, —CH₂CF₃, —CHF₂, —CF₃, —CH₂-cyclopropyl, —CH₂CH₂OH,

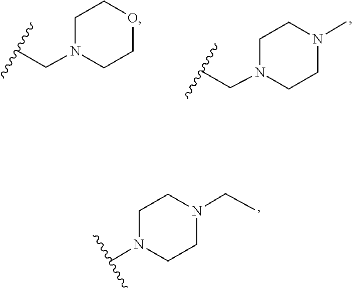

cyclopropyl, cyclobutyl,

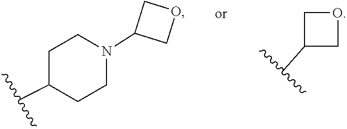 [SMILES: *C1CCN(C2COC2)CC1 ; *C1COC1]

Aspect 181. The compound according to any one of aspects 175-180, wherein Q¹ is C—H; R⁴ is —CH₃; Q³, Q⁴, and Q⁵ are each independently N or C—R^{3B}; and each R^{3B} is independently H or —CH₃.

Aspect 182. The compound according to any one of aspects 175-180, wherein Q¹ is N; R⁴ is —CH₃, each Q³, Q⁴, and Q⁵ is N or C—R^{3B}; and each R^{3B} is independently H or —CH₃.

Aspect 183. The compound according to any one of aspects 125-182, wherein E is

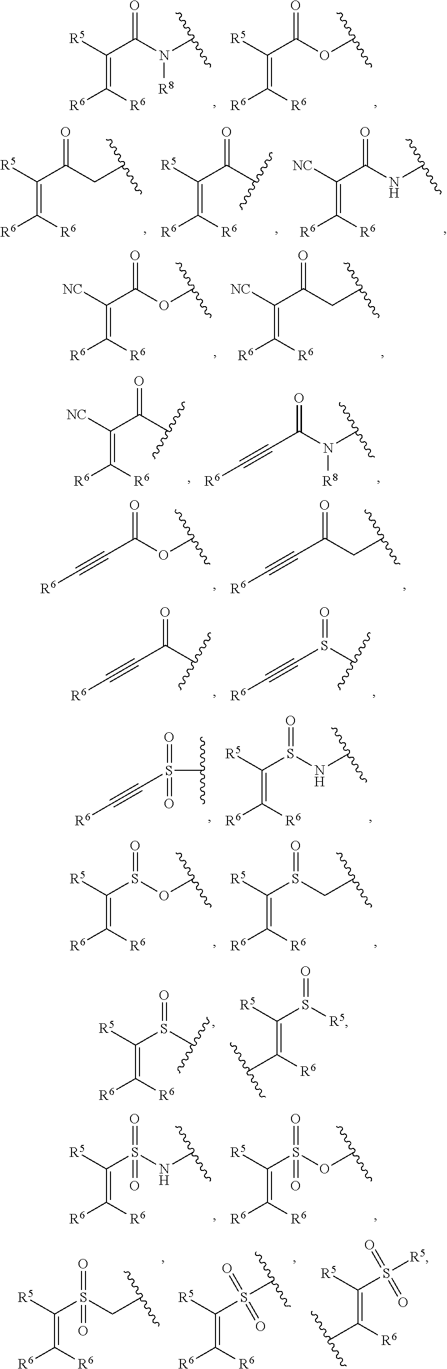

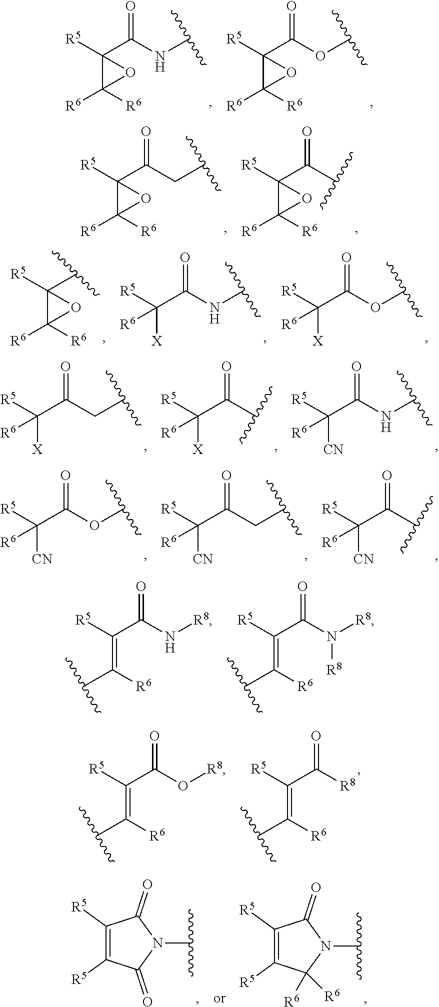

wherein each R⁵ and each R⁶ is independently H, optionally substituted C₁-C₆alkyl, or halogen; R⁸ is H or C₁-C₆alkyl; and X is halogen.

Aspect 184. The compound according to aspect 183, wherein E is

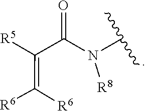

Aspect 185. The compound according to aspect 184 wherein R⁵ is H, —CH₃, or —Cl; each R⁶ is independently H or optionally substituted C₁-C₆alkyl, and each R⁸ is independently H or —CH₃.

Aspect 186. The compound according to aspect 183, wherein E is

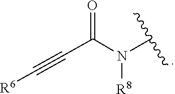

Aspect 187. The compound according to aspect 186 wherein R⁶ is H and R⁸ is H.

Aspect 188. The compound according to aspect 186 wherein R⁶ is —CH₃ and R⁸ is H or —CH₃.

Aspect 189. A pharmaceutically acceptable salt of a compound of any one of aspects 125-188.

Aspect 190. A pharmaceutical composition comprising a compound of any one aspects 125-188 and a pharmaceutically acceptable excipient.

Aspect 191. A method of treating cancer in a subject comprising administering to the subject a therapeutically effective amount of a compound of any one of aspects 125 to 188, or a pharmaceutically acceptable salt thereof.

Aspect 192. The method of aspect 191, wherein the cancer is urothelial carcinoma, breast carcinoma, endometrial adenocarcinoma, ovarian carcinoma, primary glioma, cholangiocarcinoma, gastric adenocarcinoma, non-small cell lung carcinoma, pancreatic exocrine carcinoma, oral, prostate, bladder, colorectal carcinoma, renal cell carcinoma, neuroendocrine carcinoma, myeloproliferative neoplasms, head and neck (squamous), melanoma, leiomyosarcoma, or a sarcoma.

Aspect 193. The method of aspect 191, wherein the cancer is urothelial carcinoma, breast carcinoma, endometrial cancer, endometrial adenocarcinoma, ovarian carcinoma, primary glioma, cholangiocarcinoma, gastric adenocarcinoma, non-small cell lung carcinoma, pancreatic exocrine carcinoma, oral, prostate, bladder, colorectal carcinoma, renal cell carcinoma, neuroendocrine carcinoma, myeloproliferative neoplasms, head and neck (squamous), melanoma, leiomyosarcoma, or a sarcoma.

1. A compound of formula I: 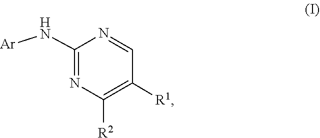
or a pharmaceutically acceptable salt thereof,
wherein:
Ar is a 6-10-membered aryl group or a 5-10-membered heteroaryl group, each optionally substituted;
R¹ is H, F, Cl, Br, C₁₋₆alkyl, CN, C₁₋₆haloalkyl, —C(O)NH₂, —C(O)NH(C₁₋₆alkyl), or —C(O)N(C₁₋₆alkyl)₂;
R² is a:
9-membered bicyclic heteroaryl comprising a 6-5-fused ring system wherein the 5-membered ring contains at least one nitrogen atom and the 6 membered ring is substituted with at least one E; or
13-membered tricyclic heteroaryl comprising a 6-5-6-fused ring system wherein the 5-membered ring contains at least one nitrogen atom and one of the 6 membered rings is substituted with at least one E; or
12- or 13-membered tricyclic group comprising a 5- or 6-membered cycloalkyl or heterocycloalkyl ring fused to a 9-membered bicyclic heteroaryl comprising a 6-5-fused ring system wherein the 5-membered ring contains at least one nitrogen atom and the 6 membered ring is substituted with at least one E;
wherein E is an electrophilic moiety that is capable of reacting with a nucleophile so as to form a covalent bond between an atom of the nucleophile and an atom of the electrophilic moiety.

2. The compound according to claim 1, wherein R² is a 9-membered bicyclic heteroaryl comprising a 6-5-fused ring system wherein the 5-membered ring contains at least one nitrogen atom and the 6 membered ring is substituted with at least one E, wherein E is an electrophilic moiety that is capable of reacting with a nucleophile so as to form a covalent bond between an atom of the nucleophile and an atom of the electrophilic moiety.

3. The compound according to claim 1, wherein R² is 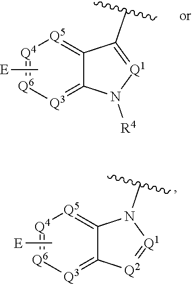
wherein
Q¹ is N or C—R^{3A};
Q² is N or C—R^{3A};
R^{3A} is H, C₁-C₆alkyl, —CN, —C₁-C₆haloalkyl, halo, —CON(R)₂, —NR₂, cycloalkyl, —CH₂cycloalkyl, —COR, —CH(OH)R, —CO-cycloalkyl, —CH(OH)cycloalkyl, —SR, or —SO₂R;
each R is independently H, or C₁-C₆alkyl;
or, when Q¹ is C—R^{3A} and Q² is C—R^{3A}, the R^{3A} of Q¹ and the R^{3A} of Q², together with the carbon atoms to which they are attached, for a 5- or 6-membered cycloalkyl or heterocycloalkyl ring, or a phenyl ring;
Q³, Q⁴, Q⁵, and Q⁶ are each independently N or C—R^{3B};
each R^{3B} is independently H, —C₁-C₆alkyl, —C₁-C₆haloalkyl, —CN, C₃-C₇cycloalkyl, or E, provided that at least one of Q³, Q⁴, Q⁵, and Q⁶ is C-E, wherein E is an electrophilic moiety that is capable of reacting with a nucleophile so as to form a covalent bond between an atom of the nucleophile and an atom of the electrophilic moiety; and
R⁴ is C₁-C₆alkyl, C₁-C₆haloalkyl, C₃-C₇cycloalkyl, or SO₂(C₁-C₆alkyl);
or, when Q¹ is C—R^{3A}, the R^{3A} of Q¹ and R⁴, together with the atoms to which they are attached, form a 5- or 6-membered heterocycloalkyl ring.

4. The compound according to claim 3, wherein R² is 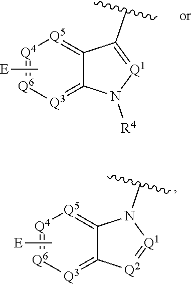
wherein
Q¹ is N or C—H;
Q² is N or C—R^{3A};
R^{3A} is H, C₁-C₆alkyl, —CN, —C₁-C₆haloalkyl, halo, —CON(R)₂, —NR₂, cycloalkyl, —CH₂cycloalkyl, —COR, —CH(OH)R, —CO-cycloalkyl, —CH(OH)cycloalkyl, —SR, or —SO₂R;
each R is independently H, or C₁-C₆alkyl;
Q³, Q⁴, Q⁵, and Q⁶ are each independently N or C—R^{3B};
each R^{3B} is independently H, —C₁-C₆alkyl, —C₁-C₆haloalkyl, —CN, C₃-C₇cycloalkyl, or E, provided that at least one of Q³, Q⁴, Q⁵, and Q⁶ is C-E, and
R⁴ is C₁-C₆alkyl, C₁-C₆haloalkyl, C₃-C₇cycloalkyl, or SO₂(C₁-C₆alkyl).

5. The compound according to claim 4, wherein R² is

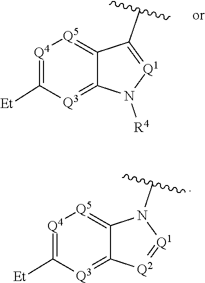

6. The compound according to claim 1, wherein the compound of formula I is a compound of formula IA: 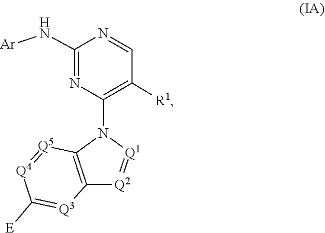
or a pharmaceutically acceptable salt thereof, wherein
Q¹ is N or C—R^{3A};
Q² is N or C—R^{3A};
R^{3A} is H, C₁-C₆alkyl, —CN, —C₁-C₆haloalkyl, halo, —CON(R)₂, —NR₂, cycloalkyl, —CH₂cycloalkyl, —COR, —CH(OH)R, —CO-cycloalkyl, —CH(OH)cycloalkyl, —SR, or —SO₂R;
each R is independently H, or C₁-C₆alkyl;
Q³, Q⁴, and Q⁵ are each independently N or C—R^{3B}; and
each R^{3B} is independently H, —C₁-C₆alkyl, —C₁-C₆haloalkyl, —CN, or C₃-C₇cycloalkyl.

7.-9. (canceled)

10. The compound according to claim 1, wherein the compound of formula I is a compound of formula IB: 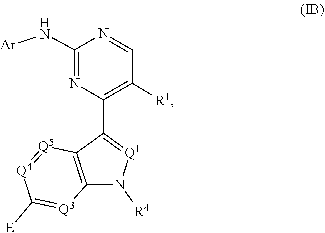
or a pharmaceutically acceptable salt thereof,
Q¹ is N or C—R^{3A};
R^{3A} is H, C₁-C₆alkyl, —CN, —C₁-C₆haloalkyl, halo, —CON(R)₂, —NR₂, cycloalkyl, —CH₂cycloalkyl, —COR, —CH(OH)R, —CO-cycloalkyl, —CH(OH)cycloalkyl, —SR, or —SO₂R;
each R is independently H or C₁-C₆alkyl;
Q³, Q⁴, and Q⁵ are each independently N or C—R^{3B};
each R^{3B} is independently H, —C₁-C₆alkyl, —C₁-C₆haloalkyl, —CN, or C₃-C₇cycloalkyl; and
R⁴ is C₁-C₆alkyl, C₁-C₆haloalkyl, C₃-C₇cycloalkyl, or SO₂(C₁-C₆alkyl).

11.-13. (canceled)

14. The compound according to claim 1, wherein R¹ is H, —Cl, —F, —CH₃, or —CN.

15.-19. (canceled)

20. The compound according to claim 1, wherein Ar is an optionally substituted 6-10-membered aryl group.

21. (canceled)

22. (canceled)

23. The compound according to claim 1, wherein Ar is an optionally substituted 5-10-membered heteroaryl group.

24. The compound according to claim 23, wherein the 5-10-membered heteroaryl group is a pyridinyl, a pyrazolyl, a triazolyl, an imidazolyl, a pyrazolopyrimidine, or a triazolopyridine.

25. The compound according to claim 23, wherein the 5-10-membered heteroaryl group is substituted with one or more optionally substituted C₁-C₆alkyl, optionally substituted C₃-C₆cycloalkyl, optionally substituted 4-6-membered heterocycloalkyl, —N(C₁-C₆alkyl)(optionally substituted C₁-C₆alkyl), —O(optionally substituted C₁-C₆alkyl), —CN, halogen, or —C(O)—O(optionally substituted C₁-C₆alkyl).

26. The compound according to claim 25, wherein the optionally substituted C₁-C₆alkyl is —CH₃, —CH₂CH₃, isopropyl, —CH₂CF₃, —CHF₂, —CF₃, —CH₂-cyclopropyl, —CH₂-(optionally substituted 5 or 6-membered heterocycloalkyl), —CH₂CH₂OH, —CH₂CH(OH)CH₃, —CH₂C(OH)(CH₃)₂, —C(CN)(CH₃)₂, —CH₂CH₂CH₂OH, —CH₂CH₂OCH₃, —CH₂CH₂OCH₂CH₃, —CH₂CH₂OCH(CH₃)₂, CH₂CH(OCH₃)CH₃, —CH₂CHF₂, —CH₂CH₂N(CH₃)₂, —CH₂CH₂-(optionally substituted 5 or 6-membered heterocycloalkyl), or —CH₂-optionally substituted cyclopropyl.

27.-40. (canceled)

41. The compound according to claim 6, wherein the compound of formula IA is a compound of formula IA-2: 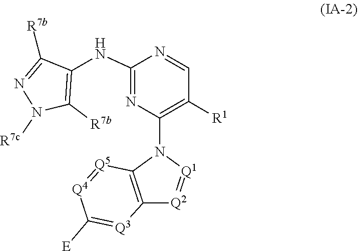
wherein each R^{7b} is independently H, —CH₃, —CH₂CH₃, isopropyl, —CH₂CF₃, —CHF₂, —CF₃, —CH₂-cyclopropyl, —CH₂CH₂OH, 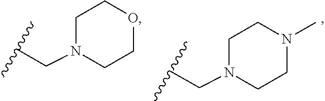
cyclopropyl, cyclobutyl, 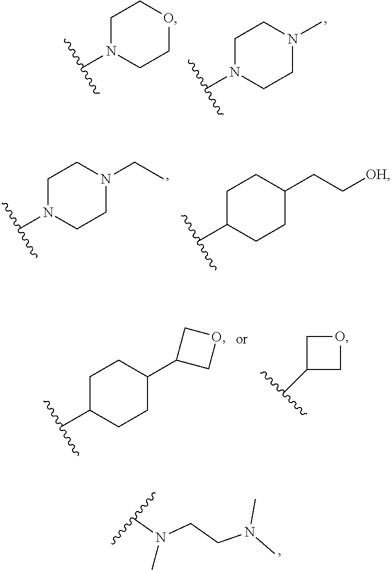
—OCH₃, —OCH₂CH₃, 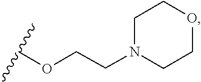 [SMILES: *OCCN1CCOCC1]
—CN, or —F;
and R^{7c} is H, —CH₃, —CH₂CH₃, isopropyl, —CH₂CF₃, —CHF₂, —CF₃, —CH₂-cyclopropyl, —CH₂CH₂OH, is —CH₂CHF₂; —CH₂—CH(OH)—CH₃, —CH₂C(OH)(CH₃)₂, —CH₂CH₂CH₂OH, —C(CH₃)₂—CN, —CH₂CH₂OCH₃, —CH₂CH₂OCH₂CH₃, —CH₂CH₂OCH(CH₃)₂, —CH₂CH₂OCHF₂, —CH₂CH₂N(CH₃)₂, 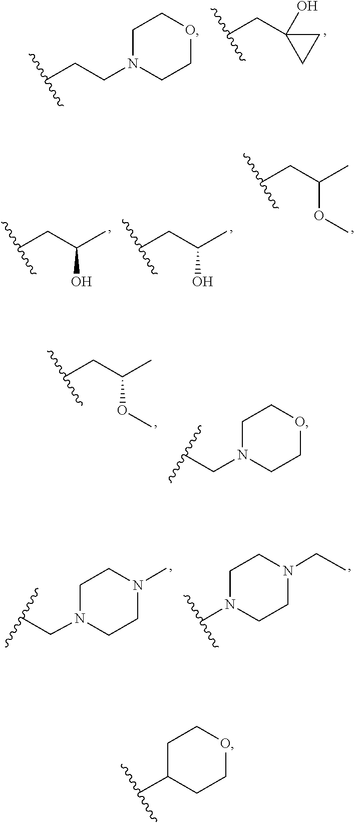
cyclopropyl, cyclobutyl,

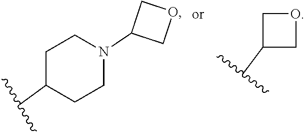 [SMILES: *C1CCN(C2COC2)CC1 ; *C1COC1]

42.-51. (canceled)

52. The compound according to claim 10, wherein the compound of formula IB is a compound of formula IB-2: 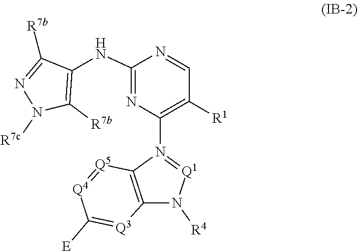
wherein each R^{7b} is independently H, —CH₃, —CH₂CH₃, isopropyl, —CH₂CF₃, —CHF₂, —CF₃, —CH₂-cyclopropyl, —CH₂CH₂OH, 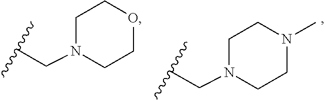
cyclopropyl, cyclobutyl, 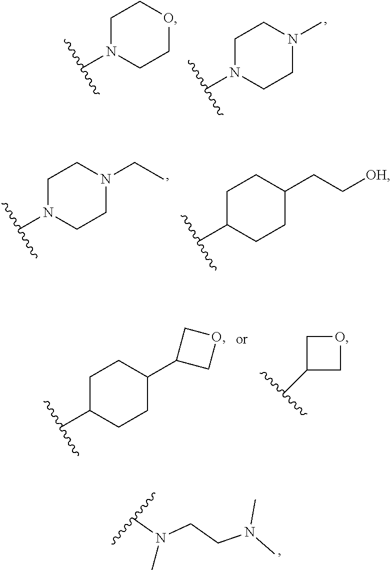
—OCH₃, —OCH₂CH₃, 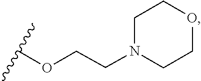 [SMILES: *OCCN1CCOCC1]
or —F;
and R^{7c} is H, —CH₃, —CH₂CH₃, isopropyl, —CH₂CF₃, —CHF₂, —CF₃, —CH₂-cyclopropyl, —CH₂CH₂OH, 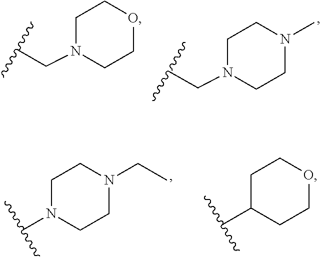
cyclopropyl, cyclobutyl, —CH₂CHF₂,

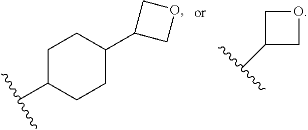 [SMILES: *C1CCC(C2COC2)CC1 ; *C1COC1]

53.-58. (canceled)

59. The compound according to claim 1, wherein E is 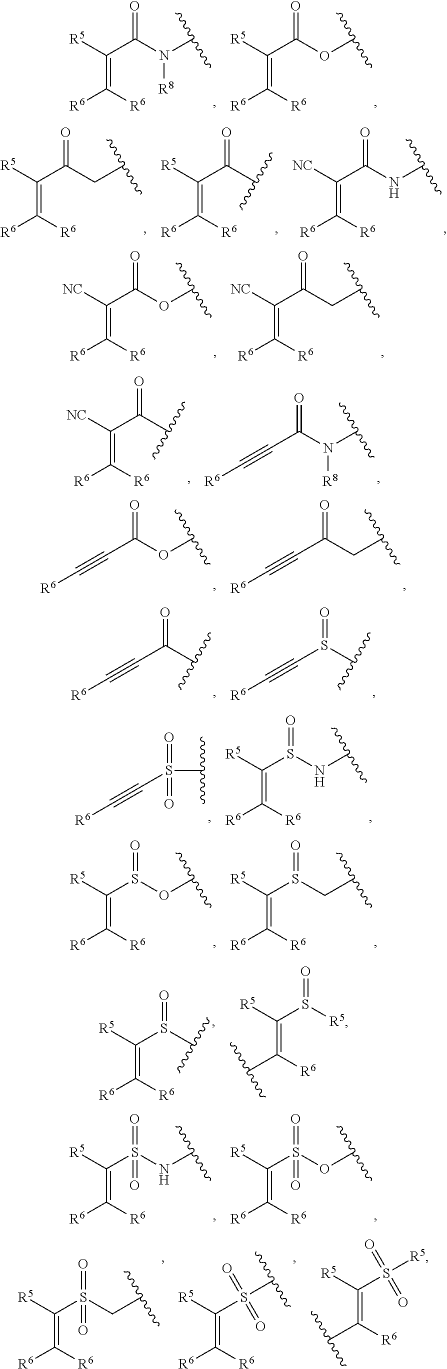 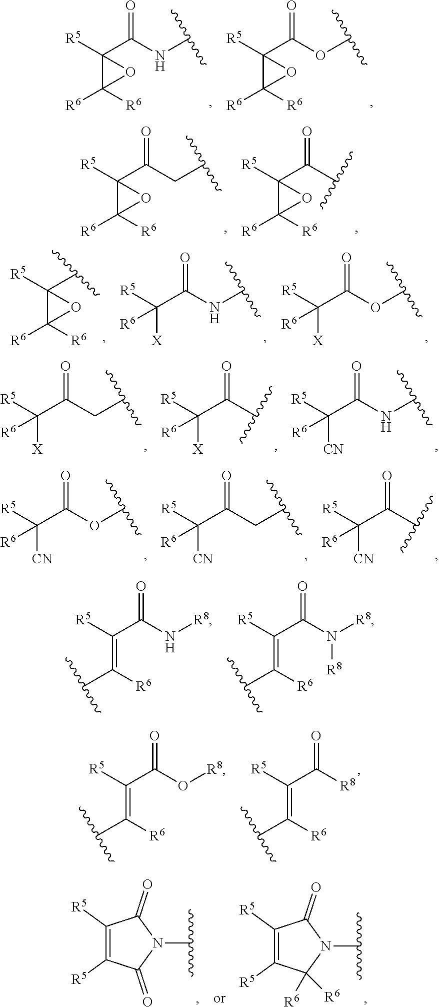
wherein each R⁵ and each R⁶ is independently H, optionally substituted C₁-C₆alkyl, or halogen; R⁸ is H or C₁-C₆alkyl; and X is halogen.

60. The compound according to claim 59, wherein E is

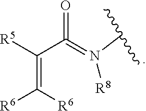

61. The compound according to claim 60 wherein R⁵ is H, —CH₃, or —C₁; each R⁶ is independently H or optionally substituted C₁-C₆alkyl, and R⁸ is H or —CH₃.

62.-65. (canceled)

66. A pharmaceutical composition comprising a compound of claim 1 and a pharmaceutically acceptable excipient.

67. A method of treating cancer in a subject comprising administering to the subject a therapeutically effective amount of a compound of claim 1, or a pharmaceutically acceptable salt thereof.

68. (canceled)

69. The method of claim 67, wherein the cancer is urothelial carcinoma, breast carcinoma, endometrial cancer, endometrial adenocarcinoma, ovarian carcinoma, primary glioma, cholangiocarcinoma, gastric adenocarcinoma, non-small cell lung carcinoma, pancreatic exocrine carcinoma, oral, prostate, bladder, colorectal carcinoma, renal cell carcinoma, neuroendocrine carcinoma, myeloproliferative neoplasms, head and neck (squamous), melanoma, leiomyosarcoma, or a sarcoma.

70. The compound according to claim 1, or a pharmaceutically acceptable salt thereof, wherein the compound is:
N-[3-methyl-1-[5-chloro-2-(4-morpholinoanilino)pyrimidin-4-yl]indol-5-yl]prop-2-enamide;
N-[3-[5-Fluoro-2-(4-morpholinoanilino)pyrimidin-4-yl]-1-methyl-indol-6-yl]prop-2-enamide;
N-[1-[5-chloro-2-[4-(4-methylpiperazin-1-yl)anilino]pyrimidin-4-yl]indol-5-yl]prop-2-enamide;
N-[1-[5-chloro-2-[(1-methylpyrazol-4-yl)amino]pyrimidin-4-yl]indol-5-yl]prop-2-enamide;
N-[1-[5-chloro-2-[4-[2-(dimethylamino)ethyl-methyl-amino]anilino]pyrimidin-4-yl]indol-5-yl]prop-2-enamide;
N-[1-[5-chloro-2-[4-(4-methylpiperazin-1-yl)anilino]pyrimidin-4-yl]indol-5-yl]propanamide;
N-[4-methyl-1-[5-methyl-2-[3-(2-morpholinoethoxy)anilino]pyrimidin-4-yl]indol-5-yl]prop-2-enamide;
N-[1-[5-chloro-2-[3-(2-morpholinoethoxy)anilino]pyrimidin-4-yl]indol-5-yl]prop-2-enamide;
N-[1-[5-chloro-2-[3-(2-morpholinoethoxy)anilino]pyrimidin-4-yl]-4-methyl-indol-5-yl]prop-2-enamide;
N-[3-methyl-1-[5-methyl-2-[(1-methylpyrazol-4-yl)amino]pyrimidin-4-yl]indol-5-yl]prop-2-enamide;
N-[3-methyl-1-[5-methyl-2-[3-(2-morpholinoethoxy)anilino]pyrimidin-4-yl]indol-5-yl]prop-2-enamide;
N-[1-[5-chloro-2-[4-(2-morpholinoethoxy)anilino]pyrimidin-4-yl]indol-5-yl]prop-2-enamide;
N-[1-[5-chloro-2-(2-ethoxy-4-morpholino-anilino)pyrimidin-4-yl]indol-5-yl]prop-2-enamide;
N-[1-[5-chloro-2-[(3-methoxy-1-methyl-pyrazol-4-yl)amino]-pyrimidin-4-yl]indol-5-yl]prop-2-enamide;
N-[1-[5-chloro-2-[2-(2-morpholinoethoxy)anilino]pyrimidin-4-yl]indol-5-yl]prop-2-enamide;
N-[1-[5-chloro-2-[(3-ethoxy-1-methyl-pyrazol-4-yl)amino]pyrimidin-4-yl]indol-5-yl]prop-2-enamide;
N-[1-[5-chloro-2-(4-morpholinoanilino)pyrimidin-4-yl]indol-5-yl]prop-2-enamide;
N-[1-[5-chloro-2-(3-fluoro-4-morpholino-anilino)pyrimidin-4-yl]indol-5-yl]prop-2-enamide;
N-[1-[5-chloro-2-(3-fluoro-4-morpholino-anilino)pyrimidin-4-yl]-4-methyl-indol-5-yl]prop-2-enamide;
N-[1-[5-chloro-2-[(1-methylpyrazol-4-yl)amino]pyrimidin-4-yl]-4-methyl-indol-5-yl]prop-2-enamide;
N-[3-methyl-1-[5-methyl-2-[4-(2-morpholinoethoxy)anilino]pyrimidin-4-yl]indol-5-yl]prop-2-enamide;
N-[1-[5-chloro-2-(2-ethoxyanilino)pyrimidin-4-yl]indol-5-yl]prop-2-enamide;
N-[1-[5-chloro-2-[4-(2-morpholinoethoxy)anilino]pyrimidin-4-yl]-4-methyl-indol-5-yl]prop-2-enamide;
N-[1-[5-chloro-2-(3-fluoro-4-morpholino-anilino)pyrimidin-4-yl]-3-methyl-indol-5-yl]prop-2-enamide;
N-[1-[5-chloro-2-(3,4-dimethoxyanilino)pyrimidin-4-yl]-3-methyl-indol-5-yl]prop-2-enamide;
N-[1-[5-chloro-2-(4-morpholinoanilino)pyrimidin-4-yl]-3-methyl-indol-5-yl]prop-2-enamide;
N-[1-[5-chloro-2-[(1-methylpyrazol-4-yl)amino]pyrimidin-4-yl]-3-methyl-indol-5-yl]prop-2-enamide;
N-[1-[5-chloro-2-[(3-methoxy-1-methyl-pyrazol-4-yl)amino]pyrimidin-4-yl]-3-methyl-indol-5-yl]prop-2-enamide;
N-[3-[5-chloro-2-(4-morpholinoanilino)pyrimidin-4-yl]-1-methyl-indol-6-yl]prop-2-enamide;
N-[1-[5-chloro-2-[(1-methylimidazol-4-yl)amino]pyrimidin-4-yl]-3-methyl-indol-5-yl]prop-2-enamide;
N-[1-[5-chloro-2-(2-ethoxy-6-methyl-anilino)pyrimidin-4-yl]-3-methyl-indol-5-yl]prop-2-enamide;
N-[3-[5-chloro-2-[(1-methylpyrazol-4-yl)amino]pyrimidin-4-yl]-1-methyl-indol-6-yl]prop-2-enamide;
N-[3-[5-chloro-2-(3,4-dimethoxyanilino)pyrimidin-4-yl]-1-methyl-indol-6-yl]prop-2-enamide;
N-[1-[5-cyano-2-[(1-methylpyrazol-4-yl)amino]pyrimidin-4-yl]-3-methyl-indol-5-yl]prop-2-enamide;
N-[1-[5-cyano-2-(2-ethoxy-6-methyl-anilino)pyrimidin-4-yl]-3-methyl-indol-5-yl]prop-2-enamide;
N-[1-[5-chloro-2-[[1-[1-(oxetan-3-yl)-4-piperidyl]pyrazol-4-yl]amino]pyrimidin-4-yl]-3-methyl-indol-5-yl]prop-2-enamide;
N-[1-[5-chloro-2-[(1,3-dimethylpyrazol-4-yl)amino]pyrimidin-4-yl]-3-methyl-indol-5-yl]prop-2-enamide;
N-[3-methyl-1-[5-methyl-2-[(1-methylimidazol-4-yl)amino]pyrimidin-4-yl]indol-5-yl]prop-2-enamide;
N-[1-[5-cyano-2-[(3-methoxy-1-methyl-pyrazol-4-yl)amino]pyrimidin-4-yl]-3-methyl-indol-5-yl]prop-2-enamide;
N-[1-[5-cyano-2-(4-morpholinoanilino)pyrimidin-4-yl]-3-methyl-indol-5-yl]prop-2-enamide;
N-[1-[5-cyano-2-(2-ethoxyanilino)pyrimidin-4-yl]-3-methyl-indol-5-yl]prop-2-enamide;
N-[1-[5-chloro-2-[(1,5-dimethylpyrazol-4-yl)amino]pyrimidin-4-yl]-3-methyl-indol-5-yl]prop-2-enamide;
N-[1-[5-chloro-2-[(1,3,5-trimethylpyrazol-4-yl)amino]pyrimidin-4-yl]-3-methyl-indol-5-yl]prop-2-enamide;
N-[1-[5-chloro-2-[[1-(1-ethyl-4-piperidyl)pyrazol-4-yl]amino]-pyrimidin-4-yl]-3-methyl-indol-5-yl]prop-2-enamide;
N-[3-[5-chloro-2-(pyrazolo[1,5-a]pyrimidin-6-ylamino)pyrimidin-4-yl]-1-methyl-indol-6-yl]prop-2-enamide;
N-[3-methyl-1-[5-methyl-2-(4-morpholinoanilino)pyrimidin-4-yl]indol-5-yl]prop-2-enamide;
N-[3-[5-chloro-2-[(6-morpholino-3-pyridyl)amino]pyrimidin-4-yl]-1-methyl-indol-6-yl]prop-2-enamide;
N-[1-[2-(3-methoxy-4-morpholino-anilino)-5-methyl-pyrimidin-4-yl]-3-methyl-indol-5-yl]prop-2-enamide;
N-[1-[5-chloro-2-([1,2,4]triazolo[1,5-a]pyridin-6-ylamino)pyrimidin-4-yl]-3-methyl-indol-5-yl]prop-2-enamide;
N-[3-[5-chloro-2-[3-methoxy-4-(4-methylpiperazin-1-yl)anilino]-pyrimidin-4-yl]-1-methyl-indol-6-yl]prop-2-enamide;
N-[3-methyl-1-[5-methyl-2-[(2-methylpyrazol-3-yl)amino]-pyrimidin-4-yl]indol-5-yl]prop-2-enamide;
N-[3-[5-chloro-2-[4-(4-methylpiperazin-1-yl)anilino]-pyrimidin-4-yl]-1-methyl-indol-6-yl]prop-2-enamide;
N-[3-[5-chloro-2-(3-methoxy-4-morpholino-anilino)pyrimidin-4-yl]-1-methyl-indol-6-yl]prop-2-enamide;
N-[1-methyl-3-[5-methyl-2-(4-morpholinoanilino)pyrimidin-4-yl]indol-6-yl]prop-2-enamide;
N-[1-methyl-3-[2-(4-morpholino-anilino)pyrimidin-4-yl]indol-6-yl]prop-2-enamide;
N-[3-methyl-1-[2-(4-morpholinoanilino)pyrimidin-4-yl]indol-5-yl]prop-2-enamide;
N-[1-[5-chloro-2-[(2-methylpyrazol-3-yl)amino]pyrimidin-4-yl]-3-methyl-indol-5-yl]prop-2-enamide;
N-[1-[5-chloro-2-[(2,5-dimethylpyrazol-3-yl)amino]-pyrimidin-4-yl]-3-methyl-indol-5-yl]prop-2-enamide;
N-[3-[5-fluoro-2-(4-morpholino-anilino)pyrimidin-4-yl]-1-methyl-indol-6-yl]prop-2-enamide;
N-[3-[5-chloro-2-[4-[2-(dimethylamino)ethyl-methyl-amino]-3-methoxy-anilino]-pyrimidin-4-yl]-1-methyl-indol-6-yl]prop-2-enamide;
N-[3-[5-chloro-2-[[6-(4-methylpiperazin-1-yl)-3-pyridyl]amino]pyrimidin-4-yl]-1-methyl-indol-6-yl]prop-2-enamide;
N-[1-[5-chloro-2-(4-morpholinoanilino)-pyrimidin-4-yl]-3,6-dimethyl-indol-5-yl]prop-2-enamide;
N-[1-[5-fluoro-2-[(1-methylpyrazol-4-yl)amino]pyrimidin-4-yl]-3-methyl-indol-5-yl]prop-2-enamide;
N-[3-[5-chloro-2-[(2-methylpyrazol-3-yl)amino]pyrimidin-4-yl]-1-methyl-indol-6-yl]prop-2-enamide;
N-[3-[5-chloro-2-[(2,5-dimethylpyrazol-3-yl)amino]-pyrimidin-4-yl]-1-methyl-indol-6-yl]prop-2-enamide;
N-[1-[2-[(2,5-dimethylpyrazol-3-yl)amino]-5-methyl-pyrimidin-4-yl]-3-methyl-indol-5-yl]prop-2-enamide;
N-[1-[5-chloro-2-[4-[(4-methylpiperazin-1-yl)methyl]-anilino]pyrimidin-4-yl]-3-methyl-indol-5-yl]prop-2-enamide;
N-[1-[5-chloro-2-[3-methyl-4-(morpholinomethyl)anilino]pyrimidin-4-yl]-3-methyl-indol-5-yl]prop-2-enamide;
N-[1-[5-fluoro-2-(4-morpholino-anilino)pyrimidin-4-yl]-3-methyl-indol-5-yl]prop-2-enamide;
N-[1-[5-chloro-2-[3-fluoro-4-(morpholinomethyl)anilino]pyrimidin-4-yl]-3-methyl-indol-5-yl]prop-2-enamide;
N-[1-[5-fluoro-2-[(2-methylpyrazol-3-yl)amino]pyrimidin-4-yl]-3-methyl-indol-5-yl]prop-2-enamide;
N-[1-[5-chloro-2-[(3-cyano-1-methyl-pyrazol-4-yl)amino]pyrimidin-4-yl]-3-methyl-indol-5-yl]prop-2-enamide;
N-[1-[5-chloro-2-[(1-methylpyrazol-4-yl)amino]pyrimidin-4-yl]-3,7-dimethyl-indol-5-yl]prop-2-enamide;
N-[1-[5-chloro-2-[(3-methyltriazol-4-yl)amino]pyrimidin-4-yl]-3-methyl-indol-5-yl]prop-2-enamide;
N-[1-[5-chloro-2-[3-(4-methylpiperazin-1-yl)anilino]-pyrimidin-4-yl]-3-methyl-indol-5-yl]prop-2-enamide;
N-[1-[5-chloro-2-(4-morpholinoanilino)pyrimidin-4-yl]-3,7-dimethyl-indol-5-yl]prop-2-enamide;
N-[1-[5-chloro-2-[4-(morpholinomethyl)anilino]pyrimidin-4-yl]-3-methyl-indol-5-yl]prop-2-enamide;
N-[1-[5-fluoro-2-[(2-methylpyrazol-3-yl)amino]pyrimidin-4-yl]-3-methyl-indazol-5-yl]prop-2-enamide;
N-[3-methyl-1-[5-methyl-2-[(2-methylpyrazol-3-yl)amino]pyrimidin-4-yl]indazol-5-yl]prop-2-enamide;
N-[1-[5-chloro-2-[3-(morpholinomethyl)anilino]pyrimidin-4-yl]-3-methyl-indol-5-yl]prop-2-enamide;
N-[3-methyl-1-[2-[(1-methylpyrazol-4-yl)amino]pyrimidin-4-yl]indol-5-yl]prop-2-enamide;
N-[1-[5-fluoro-2-[(2-methylpyrazol-3-yl)amino]pyrimidin-4-yl]-3-methyl-pyrrolo[2,3-b]pyridin-5-yl]prop-2-enamide;
N-[3-methyl-1-[5-methyl-2-[(1-methylpyrazol-3-yl)amino]pyrimidin-4-yl]indol-5-yl]prop-2-enamide;
N-[3-methyl-1-[2-[(2-methylpyrazol-3-yl)amino]pyrimidin-4-yl]indol-5-yl]prop-2-enamide;
N-[3-methyl-1-[2-[(2-methylpyrazol-3-yl)amino]pyrimidin-4-yl]pyrrolo[2,3-b]pyridin-5-yl]prop-2-enamide;
N-[1-[5-chloro-2-[(1-methylpyrazol-4-yl)amino]pyrimidin-4-yl]-3,7-dimethyl-indol-5-yl]prop-2-enamide;
N-[3-methyl-1-[5-methyl-2-[(2-methylpyrazol-3-yl)amino]pyrimidin-4-yl]pyrrolo[2,3-b]pyridin-5-yl]prop-2-enamide;
N-[1-[5-chloro-2-[3-fluoro-4-[(4-methylpiperazin-1-yl)methyl]-anilino]pyrimidin-4-yl]-3-methyl-indol-5-yl]prop-2-enamide;
N-[3-methyl-1-[2-[(2-methylpyrazol-3-yl)amino]pyrimidin-4-yl]indazol-5-yl]prop-2-enamide;
N-[1-[5-chloro-2-[3-[4-(2-hydroxyethyl)piperazin-1-yl]anilino]pyrimidin-4-yl]-3-methyl-indol-5-yl]prop-2-enamide;
N-[1-[5-chloro-2-[[1-(2-hydroxyethyl)pyrazol-4-yl]amino]pyrimidin-4-yl]-3-methyl-indol-5-yl]prop-2-enamide;
N-[1-[5-fluoro-2-[[1-(2-hydroxyethyl)pyrazol-4-yl]amino]pyrimidin-4-yl]-3-methyl-indol-5-yl]prop-2-enamide;
N-[1-[5-chloro-2-[(6-morpholino-3-pyridyl)amino]pyrimidin-4-yl]-3-methyl-indol-5-yl]prop-2-enamide;
N-[1-methyl-3-[2-[(1-methylpyrazol-3-yl)amino]pyrimidin-4-yl]indol-6-yl]prop-2-enamide;
N-[1-[2-[(1-ethylpyrazol-3-yl)amino]-5-methyl-pyrimidin-4-yl]-3-methylindol-5-yl]prop-2-enamide;
N-[3-[2-[(1-ethylpyrazol-3-yl)amino]pyrimidin-4-yl]-1-methyl-indol-6-yl]prop-2-enamide;
N-[3-[2-[(1-ethylpyrazol-4-yl)amino]pyrimidin-4-yl]-1-methyl-indol-6-yl]prop-2-enamide;
N-[3-[2-[(1-isopropylpyrazol-4-yl)amino]pyrimidin-4-yl]-1-methyl-indol-6-yl]prop-2-enamide;
N-[3-methyl-1-[5-methyl-2-[(1-methylpyrazol-3-yl)amino]pyrimidin-4-yl]pyrrolo[2,3-b]pyridin-5-yl]prop-2-enamide;
N-[3-methyl-1-[2-[(1-methylpyrazol-3-yl)amino]pyrimidin-4-yl]pyrrolo[2,3-b]pyridin-5-yl]prop-2-enamide;
N-[1-[2-[(1-cyclopropylpyrazol-3-yl)amino]-5-methyl-pyrimidin-4-yl]-3-methyl-indol-5-yl]prop-2-enamide;
N-[1-[2-[[1-(2-hydroxyethyl)pyrazol-3-yl]amino]-5-methyl-pyrimidin-4-yl]-3-methyl-indol-5-yl]prop-2-enamide;
N-[3-[2-[[1-(2-hydroxyethyl)pyrazol-3-yl]amino]pyrimidin-4-yl]-1-methyl-indol-6-yl]prop-2-enamide;
N-[1-[2-[(1,5-dimethylpyrazol-3-yl)amino]-5-methyl-pyrimidin-4-yl]-3-methyl-indol-5-yl]prop-2-enamide;
N-[3-[2-[(1,5-dimethylpyrazol-3-yl)amino]pyrimidin-4-yl]-1-methyl-indol-6-yl]prop-2-enamide;
2-methyl-N-[3-methyl-1-[5-methyl-2-[(2-methylpyrazol-3-yl)amino]pyrimidin-4-yl]indol-5-yl]prop-2-enamide;
N-[3-[2-[[1-(cyclopropylmethyl)pyrazol-4-yl]amino]pyrimidin-4-yl]-1-methyl-indol-6-yl]prop-2-enamide;
N-[3-[2-[[1-(2-hydroxyethyl)pyrazol-4-yl]amino]pyrimidin-4-yl]-1-methyl-indol-6-yl]prop-2-enamide;
(E)-4-(dimethylamino)-N-[3-methyl-1-[5-methyl-2-[(2-methylpyrazol-3-yl)amino]pyrimidin-4-yl]indol-5-yl]but-2-enamide;
N-[3-methyl-1-[5-methyl-2-[(2-methylpyrazol-3-yl)amino]pyrimidin-4-yl]indol-5-yl]prop-2-ynamide;
N-[3-methyl-1-[5-methyl-2-[(2-methylpyrazol-3-yl)amino]pyrimidin-4-yl]indol-5-yl]ethenesulfonamide;
2-fluoro-N-[3-methyl-1-[5-methyl-2-[(2-methylpyrazol-3-yl)amino]pyrimidin-4-yl]indol-5-yl]prop-2-enamide;
N-[1-methyl-3-[2-[(5-methyl-1H-pyrazol-3-yl)amino]pyrimidin-4-yl]indol-6-yl]prop-2-enamide;
N-[3-[2-[(1-cyclopropylpyrazol-3-yl)amino]pyrimidin-4-yl]-1-methyl-indol-6-yl]prop-2-enamide;
N-[3-methyl-1-[5-methyl-2-[[i-(oxetan-3-yl)pyrazol-3-yl]amino]-pyrimidin-4-yl]pyrrolo[2,3-b]pyridin-5-yl]prop-2-enamide;
N-[1-[2-[(1-ethylpyrazol-3-yl)amino]-5-methyl-pyrimidin-4-yl]-3-methyl-pyrrolo[2,3-b]pyridin-5-yl]prop-2-enamide;
(E)-4-(dimethylamino)-N-[1-[2-[(1-ethylpyrazol-3-yl)amino]-5-methyl-pyrimidin-4-yl]-3-methyl-pyrrolo[2,3-b]pyridin-5-yl]but-2-enamide;
2-chloro-N-[3-methyl-1-[5-methyl-2-[(1-methylpyrazol-3-yl)amino]pyrimidin-4-yl]pyrrolo[2,3-b]pyridin-5-yl]prop-2-enamide;
2-fluoro-N-[3-methyl-1-[5-methyl-2-[(1-methylpyrazol-3-yl)amino]pyrimidin-4-yl]pyrrolo[2,3-b]pyridin-5-yl]prop-2-enamide;
2-methyl-N-[3-methyl-1-[5-methyl-2-[(i-methylpyrazol-3-yl)amino]pyrimidin-4-yl]pyrrolo[2,3-b]pyridin-5-yl]prop-2-enamide;
N-[1-[2-[[1-(2-hydroxyethyl)pyrazol-3-yl]amino]-5-methyl-pyrimidin-4-yl]-3-methyl-pyrrolo[2,3-b]pyridin-5-yl]prop-2-enamide;
N-[3-methyl-1-[5-methyl-2-[[1-(oxetan-3-yl)pyrazol-3-yl]amino]pyrimidin-4-yl]indol-5-yl]prop-2-enamide;
N-[1-[2-[[1-(2-hydroxyethyl)pyrazol-4-yl]amino]-5-methyl-pyrimidin-4-yl]-3-methyl-indol-5-yl]prop-2-enamide;
(E)-4-(dimethylamino)-N-[1-[2-[[1-(2-hydroxyethyl)pyrazol-4-yl]amino]-5-methyl-pyrimidin-4-yl]-3-methyl-indol-5-yl]but-2-enamide;
N-[3-methyl-1-[5-methyl-2-[(5-methyl-1H-pyrazol-3-yl)amino]pyrimidin-4-yl]indol-5-yl]prop-2-enamide;
tert-butyl 5-methyl-3-[[5-methyl-4-[3-methyl-5-(prop-2-enoylamino)pyrrolo[2,3-b]pyridin-1-yl]pyrimidin-2-yl]amino]pyrazole-1-carboxylate;
N-[1-[2-[[1-(2-hydroxyethyl)pyrazol-4-yl]amino]-5-methyl-pyrimidin-4-yl]-3-methyl-pyrrolo[2,3-b]pyridin-5-yl]prop-2-enamide;
N-[3-[2-[[1-(2-hydroxyethyl)pyrazol-4-yl]amino]-5-methyl-pyrimidin-4-yl]-1-methyl-indol-6-yl]prop-2-enamide;
N-[3-[5-chloro-2-[[1-(2-hydroxyethyl)pyrazol-4-yl]amino]pyrimidin-4-yl]-1-methyl-indol-6-yl]prop-2-enamide;
N-[3-[5-fluoro-2-[[1-(2-hydroxyethyl)pyrazol-4-yl]amino]pyrimidin-4-yl]-1-methyl-indol-6-yl]prop-2-enamide;
N-[1-[5-fluoro-2-[[1-(2-hydroxyethyl)pyrazol-4-yl]amino]pyrimidin-4-yl]-3-methyl-indol-5-yl]prop-2-ynamide;
N-[3-methyl-1-[5-methyl-2-[(2-methylpyrazol-3-yl)amino]pyrimidin-4-yl]indol-5-yl]but-2-ynamide;
N-[3-methyl-1-[5-methyl-2-[(2-methylpyrazol-3-yl)amino]pyrimidin-4-yl]pyrrolo[2,3-b]pyridin-5-yl]but-2-ynamide;
N-[3-methyl-1-[5-methyl-2-[(2-methylpyrazol-3-yl)amino]pyrimidin-4-yl]pyrrolo[2,3-b]pyridin-5-yl]prop-2-ynamide;
N-[1-[5-fluoro-2-[[1-(2-hydroxyethyl)pyrazol-4-yl]amino]pyrimidin-4-yl]-3-methyl-pyrrolo[2,3-b]pyridin-5-yl]prop-2-ynamide;
N-[1-[5-fluoro-2-[(2-methylpyrazol-3-yl)amino]pyrimidin-4-yl]-3-methyl-pyrrolo[2,3-b]pyridin-5-yl]but-2-ynamide;
N-[3-[5-chloro-2-[[1-(2-hydroxyethyl)pyrazol-4-yl]amino]pyrimidin-4-yl]-1-methyl-indol-6-yl]but-2-ynamide;
N-[3-ethyl-1-[5-methyl-2-[(1-methylpyrazol-4-yl)amino]pyrimidin-4-yl]indol-5-yl]prop-2-enamide;
N-[1-[2-[[1-(2-hydroxyethyl)pyrazol-4-yl]amino]pyrimidin-4-yl]-3-methyl-indol-5-yl]prop-2-enamide;
N-[1-[5-fluoro-2-[(2-methylpyrazol-3-yl)amino]pyrimidin-4-yl]-3-methyl-indol-5-yl]prop-2-ynamide;
N-[1-[5-fluoro-2-[(2-methylpyrazol-3-yl)amino]pyrimidin-4-yl]-3-methyl-indol-5-yl]but-2-ynamide;
N-[1-[2-[[1-(2-hydroxyethyl)pyrazol-4-yl]amino]-5-methyl-pyrimidin-4-yl]-3-methyl-indol-5-yl]but-2-ynamide;
N-[1-[2-[[1-(2-hydroxyethyl)pyrazol-4-yl]amino]-5-methyl-pyrimidin-4-yl]-3-methyl-pyrrolo[2,3-b]pyridin-5-yl]prop-2-ynamide;
N-[3-[5-fluoro-2-[[1-(2-hydroxyethyl)pyrazol-4-yl]amino]pyrimidin-4-yl]-1-methyl-indol-6-yl]prop-2-ynamide;
N-[3-[2-[[1-(2-hydroxyethyl)pyrazol-4-yl]amino]pyrimidin-4-yl]-1-methyl-indol-6-yl]prop-2-ynamide;
(E)-4-(dimethylamino)-N-[1-[5-fluoro-2-[[1-(2-hydroxyethyl)pyrazol-4-yl]amino]pyrimidin-4-yl]-3-methyl-indol-5-yl]but-2-enamide;
N-[3-[2-[[1-(2-hydroxyethyl)pyrazol-4-yl]amino]pyrimidin-4-yl]-1-methyl-indol-6-yl]but-2-ynamide;
N-[1-[2-[(1,5-dimethylpyrazol-4-yl)amino]-5-fluoro-pyrimidin-4-yl]-3-methyl-indol-5-yl]prop-2-enamide;
N-[1-[2-[(1,5-dimethylpyrazol-4-yl)amino]-5-methyl-pyrimidin-4-yl]-3-methyl-indol-5-yl]prop-2-enamide;
N-[1-[5-fluoro-2-(isothiazol-4-ylamino)pyrimidin-4-yl]-3-methyl-indol-5-yl]prop-2-enamide;
N-[1-[2-[[1-(difluoromethyl)-3-methyl-pyrazol-4-yl]amino]-5-fluoro-pyrimidin-4-yl]-3-methyl-indol-5-yl]prop-2-enamide;
N-[1-[2-[(2,4-dimethylpyrimidin-5-yl)amino]-5-fluoro-pyrimidin-4-yl]-3-methyl-indol-5-yl]prop-2-enamide;
N-methyl-N-[3-methyl-1-[5-methyl-2-[(2-methylpyrazol-3-yl)amino]pyrimidin-4-yl]indol-5-yl]prop-2-enamide;
N-methyl-N-[3-methyl-1-[5-methyl-2-[(2-methylpyrazol-3-yl)amino]pyrimidin-4-yl]indol-5-yl]but-2-ynamide;
N-[1-[5-fluoro-2-[(2-methyl-3-pyridyl)amino]pyrimidin-4-yl]-3-methyl-indol-5-yl]prop-2-enamide;
N-[3-[2-[(1,3-dimethylpyrazol-4-yl)amino]-5-fluoro-pyrimidin-4-yl]-1-methyl-indol-6-yl]prop-2-enamide;
N-[1-[5-fluoro-2-[(3-methylisoxazol-4-yl)amino]pyrimidin-4-yl]-3-methyl-indol-5-yl]prop-2-enamide;
N-[1-[2-[(1-ethyl-3-methyl-pyrazol-4-yl)amino]-5-fluoro-pyrimidin-4-yl]-3-methyl-indol-5-yl]prop-2-enamide;
N-[1-[5-fluoro-2-[(2-fluoro-3-pyridyl)amino]pyrimidin-4-yl]-3-methyl-indol-5-yl]prop-2-enamide;
N-[1-[5-fluoro-2-[[1-(2-hydroxypropyl)pyrazol-4-yl]amino]pyrimidin-4-yl]-3-methyl-indol-5-yl]prop-2-enamide;
N-[1-[2-[[1-(1-cyano-1-methyl-ethyl)-3-methyl-pyrazol-4-yl]amino]-5-fluoro-pyrimidin-4-yl]-3-methyl-indol-5-yl]prop-2-enamide;
N-[1-[5-fluoro-2-[[1-(3-hydroxypropyl)pyrazol-4-yl]amino]pyrimidin-4-yl]-3-methyl-indol-5-yl]prop-2-enamide;
N-[1-[5-fluoro-2-[(5-methylisothiazol-3-yl)amino]pyrimidin-4-yl]-3-methyl-indol-5-yl]prop-2-enamide;
N-[1-[5-fluoro-2-[[1-(2-hydroxyethyl)-3-methyl-pyrazol-4-yl]amino]pyrimidin-4-yl]-3-methyl-indol-5-yl]prop-2-enamide;
N-[1-[5-fluoro-2-[[1-(2-hydroxyethyl)-5-methyl-pyrazol-4-yl]amino]pyrimidin-4-yl]-3-methyl-indol-5-yl]prop-2-enamide;
N-[1-[5-fluoro-2-[[1-(2-methoxyethyl)pyrazol-4-yl]amino]pyrimidin-4-yl]-3-methyl-indol-5-yl]prop-2-enamide;
N-[1-[2-[[1-(2,2-difluoroethyl)-3-methyl-pyrazol-4-yl]amino]-5-fluoro-pyrimidin-4-yl]-3-methyl-indol-5-yl]prop-2-enamide;
N-[1-[5-fluoro-2-[[3-methyl-1-(2,2,2-trifluoroethyl)pyrazol-4-yl]amino]pyrimidin-4-yl]-3-methyl-indol-5-yl]prop-2-enamide;
N-[1-[2-[[1-[2-(dimethylamino)ethyl]-3-methyl-pyrazol-4-yl]amino]-5-fluoro-pyrimidin-4-yl]-3-methyl-indol-5-yl]prop-2-enamide;
N-[1-[2-[[1-[2-(dimethylamino)ethyl]-5-methyl-pyrazol-4-yl]amino]-5-fluoro-pyrimidin-4-yl]-3-methyl-indol-5-yl]prop-2-enamide;
N-[1-[2-[(1,3-dimethylpyrazol-4-yl)amino]-5-fluoro-pyrimidin-4-yl]-3-methyl-indol-5-yl]prop-2-enamide;
N-[1-methyl-3-[2-[[1-(2,2,2-trifluoroethyl)pyrazol-4-yl]amino]pyrimidin-4-yl]indol-6-yl]prop-2-enamide;
N-[3-[2-[[1-(2,2-difluoroethyl)-3-methyl-pyrazol-4-yl]amino]-5-fluoro-pyrimidin-4-yl]-1-methyl-indol-6-yl]prop-2-enamide;
N-[3-[2-[[1-(2,2-difluoroethyl)-3-methyl-pyrazol-4-yl]amino]pyrimidin-4-yl]-1-methyl-indol-6-yl]prop-2-enamide;
(E)-4-(dimethylamino)-N-[1-[2-[(1-ethyl-3-methyl-pyrazol-4-yl)amino]-5-fluoro-pyrimidin-4-yl]-3-methyl-pyrrolo[2,3-b]pyridin-5-yl]but-2-enamide;
N-[3-[2-[(1,3-dimethylpyrazol-4-yl)amino]pyrimidin-4-yl]-1-methyl-indol-6-yl]prop-2-enamide;
N-[3-[2-[(1-ethyl-3-methyl-pyrazol-4-yl)amino]pyrimidin-4-yl]-1-methyl-indol-6-yl]prop-2-enamide;
N-[1-[5-fluoro-2-[[3-methyl-1-(2-morpholinoethyl)pyrazol-4-yl]amino]pyrimidin-4-yl]-3-methyl-indol-5-yl]prop-2-enamide;
N-[1-[5-fluoro-2-[[5-methyl-1-(2-morpholinoethyl)pyrazol-4-yl]amino]pyrimidin-4-yl]-3-methyl-indol-5-yl]prop-2-enamide;
N-[1-[5-fluoro-2-[[1-[(1-hydroxycyclopropyl)methyl]pyrazol-4-yl]amino]pyrimidin-4-yl]-3-methyl-indol-5-yl]prop-2-enamide;
N-[1-[5-fluoro-2-[[1-(2-hydroxyethyl)-3-methyl-pyrazol-4-yl]amino]pyrimidin-4-yl]-3-methyl-indol-5-yl]prop-2-enamide;
N-[3-[2-[(1-ethyl-3-methyl-pyrazol-4-yl)amino]-5-fluoro-pyrimidin-4-yl]-1-methyl-indol-6-yl]prop-2-enamide;
N-[3-[2-[(1-cyclopropylpyrazol-4-yl)amino]pyrimidin-4-yl]-1-methyl-indol-6-yl]prop-2-enamide;
(E)-4-(dimethylamino)-N-[1-[2-[(1,3-dimethylpyrazol-4-yl)amino]-5-fluoro-pyrimidin-4-yl]-3-methyl-pyrrolo[2,3-b]pyridin-5-yl]but-2-enamide;
(E)-N-[1-[5-chloro-2-[(1,3-dimethylpyrazol-4-yl)amino]pyrimidin-4-yl]-3-methyl-pyrrolo[2,3-b]pyridin-5-yl]-4-(dimethylamino)but-2-enamide;
N-[1-[5-fluoro-2-[[1-(2-hydroxy-2-methyl-propyl)pyrazol-4-yl]amino]pyrimidin-4-yl]-3-methyl-indol-5-yl]prop-2-enamide;
N-[1-[5-fluoro-2-[[1-(2-hydroxy-2-methyl-propyl)-3-methyl-pyrazol-4-yl]amino]pyrimidin-4-yl]-3-methyl-indol-5-yl]prop-2-enamide;
N-[1-[5-fluoro-2-[[1-(2-hydroxy-2-methyl-propyl)-5-methyl-pyrazol-4-yl]amino]pyrimidin-4-yl]-3-methyl-indol-5-yl]prop-2-enamide;
N-[1-methyl-3-[2-[[1-(trifluoromethyl)pyrazol-4-yl]amino]pyrimidin-4-yl]indol-6-yl]prop-2-enamide;
N-[1-[5-fluoro-2-[[1-[(2R)-2-hydroxypropyl]pyrazol-4-yl]amino]pyrimidin-4-yl]-3-methyl-indol-5-yl]prop-2-enamide;
N-[1-[5-fluoro-2-[[1-[(2S)-2-hydroxypropyl]pyrazol-4-yl]amino]pyrimidin-4-yl]-3-methyl-indol-5-yl]prop-2-enamide;
N-[1-[2-[(1,3-dimethylpyrazol-4-yl)amino]-5-methyl-pyrimidin-4-yl]-3-methyl-indol-5-yl]prop-2-enamide;
N-[3-[2-[[1-(difluoromethyl)pyrazol-4-yl]amino]pyrimidin-4-yl]-1-methyl-indol-6-yl]prop-2-enamide;
N-[3-[2-[(1-cyclobutylpyrazol-4-yl)amino]pyrimidin-4-yl]-1-methyl-indol-6-yl]prop-2-enamide;
N-[1-methyl-3-[2-[[1-(oxetan-3-yl)pyrazol-4-yl]amino]pyrimidin-4-yl]indol-6-yl]prop-2-enamide;
(E)-4-(dimethylamino)-N-[1-[5-fluoro-2-[[1-[(2R)-2-hydroxypropyl]pyrazol-4-yl]amino]pyrimidin-4-yl]-3-methyl-pyrrolo[2,3-b]pyridin-5-yl]but-2-enamide;
N-[1-[5-fluoro-2-[[1-(2-hydroxyethyl)-5-methyl-pyrazol-4-yl]amino]pyrimidin-4-yl]-3-methyl-indol-5-yl]prop-2-enamide;
N-[1-[2-[[1-(2-ethoxyethyl)pyrazol-4-yl]amino]-5-fluoro-pyrimidin-4-yl]-3-methyl-indol-5-yl]prop-2-enamide;
N-[1-[5-fluoro-2-[[1-[(2R)-2-hydroxypropyl]-3-methyl-pyrazol-4-yl]amino]pyrimidin-4-yl]-3-methyl-indol-5-yl]prop-2-enamide;
N-[1-[5-fluoro-2-[[1-[(2R)-2-hydroxypropyl]-5-methyl-pyrazol-4-yl]amino]pyrimidin-4-yl]-3-methyl-indol-5-yl]prop-2-enamide;
N-[1-[5-fluoro-2-[[1-[(2S)-2-hydroxypropyl]-3-methyl-pyrazol-4-yl]amino]pyrimidin-4-yl]-3-methyl-indol-5-yl]prop-2-enamide;
N-[1-[5-fluoro-2-[[1-[(2S)-2-hydroxypropyl]-5-methyl-pyrazol-4-yl]amino]pyrimidin-4-yl]-3-methyl-indol-5-yl]prop-2-enamide;
(E)-4-(dimethylamino)-N-[1-[5-fluoro-2-[[1-[(2S)-2-hydroxypropyl]pyrazol-4-yl]amino]pyrimidin-4-yl]-3-methyl-pyrrolo[2,3-b]pyridin-5-yl]but-2-enamide;
(E)-N-[1-[5-fluoro-2-[[1-[(2R)-2-hydroxypropyl]pyrazol-4-yl]amino]pyrimidin-4-yl]-3-methyl-pyrrolo[2,3-b]pyridin-5-yl]-4-morpholino-but-2-enamide;
(E)-N-[1-[5-fluoro-2-[[1-[(2S)-2-hydroxypropyl]pyrazol-4-yl]amino]pyrimidin-4-yl]-3-methyl-pyrrolo[2,3-b]pyridin-5-yl]-4-morpholino-but-2-enamide;
(E)-4-(dimethylamino)-N-[1-[2-[(1,5-dimethylpyrazol-4-yl)amino]-5-fluoro-pyrimidin-4-yl]-3-methyl-pyrrolo[2,3-b]pyridin-5-yl]but-2-enamide;
(E)-4-(dimethylamino)-N-[1-[2-[(1,3-dimethylpyrazol-4-yl)amino]-5-methyl-pyrimidin-4-yl]-3-methyl-pyrrolo[2,3-b]pyridin-5-yl]but-2-enamide;
N-[3-[2-[(1-methylpyrazol-4-yl)amino]pyrimidin-4-yl]-1-methyl-indol-6-yl]prop-2-enamide;
N-[1-[5-fluoro-2-[[1-(2-methoxyethyl)-3-methyl-pyrazol-4-yl]amino]pyrimidin-4-yl]-3-methyl-indol-5-yl]prop-2-enamide;
N-[1-[5-fluoro-2-[[1-(2-isopropoxyethyl)pyrazol-4-yl]amino]pyrimidin-4-yl]-3-methyl-indol-5-yl]prop-2-enamide;
N-[1-[5-fluoro-2-[[1-(2-methoxypropyl)pyrazol-4-yl]amino]pyrimidin-4-yl]-3-methyl-indol-5-yl]prop-2-enamide;
N-[1-[5-fluoro-2-[[1-[(2S)-2-methoxypropyl]pyrazol-4-yl]amino]pyrimidin-4-yl]-3-methyl-indol-5-yl]prop-2-enamide;
N-[1-[5-fluoro-2-[[1-[(1-hydroxycyclopropyl)methyl]-3-methyl-pyrazol-4-yl]amino]pyrimidin-4-yl]-3-methyl-indol-5-yl]prop-2-enamide;
N-[1-[5-fluoro-2-[[1-[(1-hydroxycyclopropyl)methyl]-5-methyl-pyrazol-4-yl]amino]pyrimidin-4-yl]-3-methyl-indol-5-yl]prop-2-enamide;
N-[1-[2-(5, 6-dihydro-4H-pyrrolo[1,2-b]pyrazol-3-ylamino)-5-fluoro-pyrimidin-4-yl]-3-methyl-indol-5-yl]prop-2-enamide;
N-[1-[5-fluoro-2-[(1-tetrahydropyran-4-ylpyrazol-4-yl)amino]pyrimidin-4-yl]-3-methyl-indol-5-yl]prop-2-enamide;
N-[1-[6-[(1,3-dimethylpyrazol-4-yl)amino]-3-methyl-pyridazin-4-yl]-3-methyl-indol-5-yl]prop-2-enamide;
N-[1-[5-fluoro-2-[[1-[(2R)-2-methoxypropyl]pyrazol-4-yl]amino]pyrimidin-4-yl]-3-methyl-indol-5-yl]prop-2-enamide;
N-[1-[5-fluoro-2-[[3-methyl-1-(oxetan-3-yl)pyrazol-4-yl]amino]pyrimidin-4-yl]-3-methyl-indol-5-yl]prop-2-enamide;
N-[1-[5-fluoro-2-[[5-methyl-1-(oxetan-3-yl)pyrazol-4-yl]amino]pyrimidin-4-yl]-3-methyl-indol-5-yl]prop-2-enamide;
N-[1-[5-fluoro-2-[[1-(2-isopropoxyethyl)-3-methyl-pyrazol-4-yl]amino]pyrimidin-4-yl]-3-methyl-indol-5-yl]prop-2-enamide;
N-[1-[5-fluoro-2-[[1-(2-isopropoxyethyl)-5-methyl-pyrazol-4-yl]amino]pyrimidin-4-yl]-3-methyl-indol-5-yl]prop-2-enamide;
(E)-N-[1-[2-[(1,3-dimethylpyrazol-4-yl)amino]-5-fluoro-pyrimidin-4-yl]-3-methyl-pyrrolo[2,3-b]pyridin-5-yl]-4-morpholino-but-2-enamide;
N-[1-[5-fluoro-2-[[1-[(2R)-2-hydroxypropyl]-3-methyl-pyrazol-4-yl]amino]pyrimidin-4-yl]-3-methyl-indol-5-yl]prop-2-enamide;
N-[1-[5-fluoro-2-[(1-tetrahydrofuran-3-ylpyrazol-4-yl)amino]pyrimidin-4-yl]-3-methyl-indol-5-yl]prop-2-enamide;
N-[1-[5-fluoro-2-[[1-[(2R)-2-hydroxypropyl]-5-methyl-pyrazol-4-yl]amino]pyrimidin-4-yl]-3-methyl-indol-5-yl]prop-2-enamide;
N-[1-[6-[(1,3-dimethylpyrazol-4-yl)amino]-3-methyl-pyridazin-4-yl]-3-methyl-indol-5-yl]prop-2-enamide;
N-[3-methyl-1-[4-[(1-methylpyrazol-4-yl)amino]-1,3,5-triazin-2-yl]indol-5-yl]prop-2-enamide;
N-[3-methyl-1-[6-[(1-methylpyrazol-4-yl)amino]pyrimidin-4-yl]indol-5-yl]prop-2-enamide;
N-[1-[4-[(1,3-dimethylpyrazol-4-yl)amino]-1,3,5-triazin-2-yl]-3-methyl-indol-5-yl]prop-2-enamide;
N-[3-methyl-1-[3-methyl-6-[(1-methylpyrazol-4-yl)amino]pyridazin-4-yl]indol-5-yl]prop-2-enamide;
N-[1-[6-[(1,3-dimethylpyrazol-4-yl)amino]pyrimidin-4-yl]-3-methyl-indol-5-yl]prop-2-enamide;
N-[3-methyl-1-[5-methyl-2-[(1-methylpyrazol-4-yl)amino]-4-pyridyl]indol-5-yl]prop-2-enamide;
N-[1-[2-[(1,3-dimethylpyrazol-4-yl)amino]-5-methyl-4-pyridyl]-3-methyl-indol-5-yl]prop-2-enamide;
N-[1-[6-chloro-3-[(1,3-dimethylpyrazol-4-yl)amino]-1,2,4-triazin-5-yl]-3-methyl-indol-5-yl]prop-2-enamide;
N-[1-[5-fluoro-2-[(1-methylpyrazol-4-yl)amino]pyrimidin-4-yl]-2,3-dimethyl-indol-5-yl]prop-2-enamide;
N-[1-[5-fluoro-2-[(1-methylpyrazol-4-yl)amino]pyrimidin-4-yl]-2-methyl-indol-5-yl]prop-2-enamide;
N-[9-[5-fluoro-2-[(1-methylpyrazol-4-yl)amino]pyrimidin-4-yl]carbazol-3-yl]prop-2-enamide;
N-[4-[5-fluoro-2-[(1-methylpyrazol-4-yl)amino]pyrimidin-4-yl]-2,3-dihydro-1H-cyclopenta[b]indol-7-yl]prop-2-enamide; or
N-[9-[5-fluoro-2-[(1-methylpyrazol-4-yl)amino]pyrimidin-4-yl]-5,6,7,8-tetrahydrocarbazol-3-yl]prop-2-enamide.
